(19) United States
(12) Patent Application Publication
(10) Pub. No.: US 2026/0317344 A1
(43) Pub. Date: Oct. 15, 2026

(54) EGFR INHIBITORS FOR TREATING DISEASE

(71) Applicant: BLOSSOMHILL THERAPEUTICS, INC., San Diego, CA (US)

(72) Inventors: Jean Jingrong CUI, San Diego, CA (US); Eugene Yuanjin RUI, San Diego, CA (US); Evan W. ROGERS, San Diego, CA (US); Anindya SARKAR, San Diego, CA (US); Dayong ZHAI, San Diego, CA (US); Wei DENG, San Diego, CA (US); Jane UNG, San Diego, CA (US)

(21) Appl. No.: 19/480,596

(22) PCT Filed: May 1, 2024
(86) PCT No.: PCT/US2024/027195

§ 371 Date: Oct. 31, 2025

Related U.S. Application Data
(60) Provisional application No. 63/463,498, filed on May 2, 2023.
(60) Provisional application No. 63/636,734, filed on Apr. 20, 2024.
(60) Provisional application No. 63/637,624, filed on Apr. 23, 2024.

(51) Int. Cl. C07D 498/22; A61K 31/439; A61K 31/4545; A61K 31/5377; C07B 59/00

(52) U.S. Cl. CPC C07D 498/22; A61K 31/439; A61K 31/4545; A61K 31/5377; C07B 59/002

(57) ABSTRACT

The present disclosure relates to compounds targeting kinases such as EGFR, pharmaceutical compositions containing the compounds, and methods of using such compounds to treat disease, such as cancer.

### RELATED APPLICATIONS

This application claims the benefit of U.S. Provisional Application No. 63/463,498, filed May 2, 2023, U.S. Provisional Application No. 63/627,624, filed Jan. 31, 2024, and U.S. Provisional Application No. 63/636,734, filed Apr. 20, 2024, the entire disclosures of all of which are incorporated herein by reference.

### TECHNICAL FIELD

The present disclosure relates to compounds targeting kinases such as EGFR, pharmaceutical compositions containing the compounds, and methods of using such compounds to treat disease, such as cancer.

### BACKGROUND

Protein kinases are tightly regulated signaling proteins that orchestrate the activation of signaling cascades by phosphorylating target proteins in response to extracellular and intracellular stimuli. The human genome encodes approximately 518 protein kinases (Manning G, et al., The protein kinase complement of the human genome. Science. 2002, 298:1912-34). Dysregulation of kinase activity is associated with many diseases, including cancers, and cardiovascular, degenerative, immunological, infectious, inflammatory, and metabolic diseases (Levitzki, A., Protein kinase inhibitors as a therapeutic modality. Acc. Chem. Res. 2003, 36:462-469). The molecular bases leading to various diseases include kinase gain- and loss-of-function mutations, gene amplifications and deletions, splicing changes, and translocations (Wilson L J, et al., New Perspectives, Opportunities, and Challenges in Exploring the Human Protein Kinome. Cancer Res. 2018, 78:15-29). The critical role of kinases in cancer and other diseases makes them attractive targets for drug inventions with 62 small molecule kinase inhibitors have been approved and 55 of them for cancer targeted therapies (Roskoski R Jr, Properties of FDA-approved Small Molecule Protein Kinase Inhibitors: A 2021 Update. Pharmacol Res 2021, 165:105463). Although kinase inhibitors have achieved dramatic success in cancer targeted therapies, the development of treatment resistance has remained as a challenge for small molecule kinase inhibitors. Acquired secondary mutations within kinase domain during the treatment often lead to treatment resistance to kinase inhibitors (Pottier C, et al., Tyrosine Kinase Inhibitors in Cancer: Breakthrough and Challenges of Targeted Therapy. Cancers (Basel), 2020, 12:731). Resistance can also arise from subpopulations of tolerant/persister cells that survive in the presence of the treatment. Different processes contribute to the emergence of tolerant persister cells, including pathway rebound through the release of negative feedback loops, transcriptional rewiring mediated by chromatin remodeling and autocrine/paracrine communication among tumor cells and within the tumor microenvironment (Swayden M, et al., Tolerant/Persister Cancer Cells and the Path to Resistance to Targeted Therapy. Cells 2020, 9, 2601). Therefore, it is necessary to invent kinase inhibitors that can target not only the kinase oncogenic drivers, overcome most frequent resistance mutations, but also tolerant persister cancer cells for overcoming resistance, achieving better efficacy and longer disease control.

Non-small-cell lung cancer (NSCLC) is the leading cause of cancer mortality worldwide (World Health Organisation. Cancer Fact Sheet 2017). Activating EGFR mutations have been reported in approximately 10% to 15% of cases of adenocarcinoma in white patients and 50% of cases in Asian patients (Chan B A, Hughes B G, Targeted therapy for non-small cell lung cancer: current standards and the promise of the future. Transl Lung Cancer Res 2015; 4:36-54). The two most frequent EGFR alterations found in NSCLC tumors are short in-frame deletions in exon 19 (del19) of the EGFR gene and L858R, a single missense mutation in exon 21 (Konduri K. et al., EGFR Fusions as Novel Therapeutic Targets in Lung Cancer. Cancer Discovery 2016, 6:601-11).

The first-generation reversible EGFR inhibitors, erlotinib and gefitinib are superior to chemotherapy in patients with advanced EGFR mutation-positive (Del19 or L858R) NSCLC and have been used as first-line standard of care in this setting. However, most patients will develop resistance to gefitinib or erlotinib with 50% to 70% of tumors developing EGFR T790M gatekeeper mutation with time of treatment (Sequist L V, et al., Genotypic and histological evolution of lung cancers acquiring resistance to EGFR inhibitors. Sci Transl Med 2011; 3:75ra26). The second generation of EGFR inhibitors afatinib and dacomitinib are covalent, irreversible EGFR inhibitors that also inhibit HER2 and ERB4 of the ERB family (Li D, et al., BIBW2992, an irreversible EGFR/HER2 inhibitor highly effective in preclinical lung cancer models. Oncogene 2008; 27: 4702-11; Ou S H, Soo R A. Dacomitinib in lung cancer: a “lost generation” EGFR tyrosine-kinase inhibitor from a bygone era? Drug Des Devel Ther 2015; 9:5641-53).

Although afatinib and dacomitinib are more potent EGFR inhibitors approved as first-line therapy for advanced EGFR mutation-positive (Del19 or L858R) NSCLC with longer progression free survival time (PFS) in comparison with gefitinib and erlotinib, EGFR T790M has been developed with time of treatment with afatinib (Tanaka K, et al., Acquisition of the T790M resistance mutation during afatinib treatment in EGFR tyrosine kinase inhibitor-naive patients with non-small cell lung cancer harboring EGFR mutations. Onco-target 2017; 8:68123-30). EGFR T790M confers resistance to dacomitinib In vitro studies (Kobayashi Y, et al., EGFR T790M and C797S mutations as mechanisms of acquired resistance to dacomitinib. J Thorac Oncol 2018; 13: 727-31). The third-generation EGFR inhibitor Osimertinib is also an irreversible inhibitor targeting both EGFR activating mutations (Del19 and L858R) and T790M resistant double mutations, with selectivity over the wild-type EGFR (Finlay M R, et al., Discovery of a potent and selective EGFR inhibitor (AZD9291) of both sensitizing and T790M resistance mutations that spares the wild type form of the receptor. J Med Chem 2014; 57:8249-67). Osimertinib was first approved for patients with metastatic EGFR T790M mutation-positive NSCLC after failure of first-line EGFR inhibitors, and later approved in the first-line setting for patients with EGFR mutation-positive NSCLC following the phase III FLAURA trial with head-to-head trials comparing with erlotinib or gefitinib (Soria J C, et al., Osimertinib in untreated EGFR-mutated advanced non-small-cell lung cancer. N Engl J Med 2018; 378:113-25). The mutation C797S at the EGFR covalent binding residue with irreversible EGFR inhibitor Osimertinib has been detected in Osimertinib-resistant patients (Ramalingam S S, et al., Mechanisms of acquired resistance to first-line osimertinib: preliminary data from the phase III FLAURA study. Presented at the ESMO 2018). EGFR mutations (L858R or exon 19 deletions (Exl9del)) are classified as EGFR classic mutations, or common mutations, which showed marked efficacy with currently approved EGFR inhibitors.

Therefore, there is an urgent medical need to develop holistic EGFR inhibitors that are potent against classical EGFR mutations, including L858R, Δ746-750, Δ746-750/C979S, L858R/T790M, L858R/T790M/C979S, and D770_N771insNPG, while maintaining good selectivity over wild-type EGFR.

### SUMMARY

In one aspect, the disclosure relates to a compound of the formula I, or a pharmaceutically acceptable salt, solvate, hydrate, or cocrystal thereof,

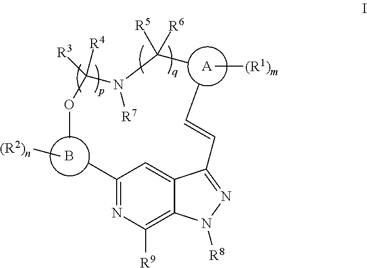

wherein R¹, R², R³, R⁴, R⁵, R⁶, R⁷, R⁸, R⁹, A, B, m, n, p, and q are as described herein.

In another aspect, the disclosure relates to a compound of the formula II, or a pharmaceutically acceptable salt, solvate, hydrate, or cocrystal thereof,

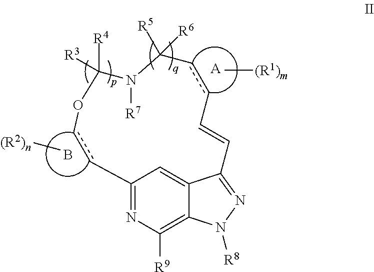

wherein R¹, R², R³, R⁴, R⁵, R⁶, R⁷, R⁸, R⁹, A, B, m, n, p, and q are as described herein.

In another aspect, the disclosure relates to a compound of the formula III, or a pharmaceutically acceptable salt, solvate, hydrate, or cocrystal thereof,

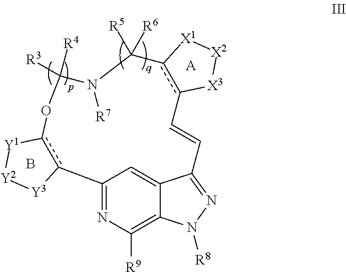

wherein X¹, X², X³, Y¹, Y², Y³, R³, R⁴, R⁵, R⁶, R⁷, R⁸, R⁹, A, B, p, and q are as described herein.

In another aspect, the disclosure relates to a compound of the formula IV, or a pharmaceutically acceptable salt, solvate, hydrate, or cocrystal thereof,

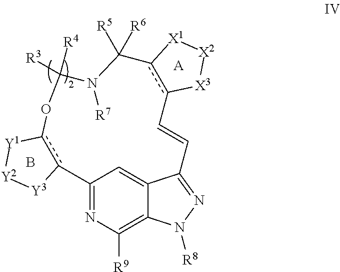

wherein X¹, X², X³, Y¹, Y², Y³, R³, R⁴, R⁵, R⁶, R⁷, R⁸, R⁹, A, and B are as described herein.

In certain embodiments of the above aspects, the compound of Formula (I)-(IV) is a compound selected from those species described or exemplified in the detailed description below.

In further aspects, the disclosure relates to a pharmaceutical composition comprising at least one compound of Formula (I)-(IV) or a pharmaceutically acceptable salt, solvate, hydrate, or cocrystal thereof. Pharmaceutical compositions according to the disclosure may further comprise a pharmaceutically acceptable excipient.

In further aspects, the disclosure relates to a compound of Formula (I)-(IV), or a pharmaceutically acceptable salt, solvate, hydrate, or cocrystal thereof, for use as a medicament.

In further aspects, the disclosure relates to a method of treating disease, such as cancer comprising administering to a subject in need of such treatment an effective amount of at least one compound of Formula (I)-(IV), or a pharmaceutically acceptable salt, solvate, hydrate, or cocrystal thereof.

In further aspects, the disclosure relates to use of a compound of Formula (I)-(IV), or a pharmaceutically acceptable salt, solvate, hydrate, or cocrystal thereof, in the preparation of a medicament for the treatment of disease, such as cancer, and the use of such compounds and salts for treatment of such diseases.

In further aspects, the disclosure relates to a method of inhibiting a tyrosine kinase, such as EGFR, including the certain mutations as described herein, comprising contacting a cell comprising one or more of kinase with an effective amount of at least one compound of Formula (I)-(IV), or a pharmaceutically acceptable salt, solvate, hydrate, or cocrystal thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I

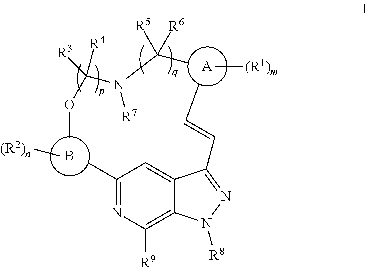

wherein
ring A and ring B are each independently a 5-membered heteroarylene;
each R¹ and R², when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5-to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R″, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{e}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R³, R⁴, R⁵, and R⁶ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5-to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{c}R^{f}, —P(O)R^{e}R^{f}, —P(O)₂R^{c}R^{f}, —P(O)NR^{e}R^{f}, —P(O)₂NR^{e}R^{f}, —P(O)OR^{e}, —P(O)₂OR^{e}, —CN, or —NO₂; or two of R³, R⁴, R⁵, and R⁶, taken together with the carbon or carbons to which they are attached, combine to form C₃-C₆ cycloalkyl or 3- to 7-membered heterocycloalkyl, wherein each hydrogen atom in C₃-C₆ cycloalkyl and 3- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁷ is H, deuterium, C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, 5- to 10-membered heteroaryl, —C(O)R^{e}, or —C(O)NR^{c}R^{d}, wherein each hydrogen atom in C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, and 5- to 10-membered heteroaryl is independently optionally substituted by —OR^{c}, —OC(O)R^{c}, —OC(O)NR^{c}R^{d}, —OC(═NR^{d})NR^{c}R^{d}, —OS(O)R^{e}, —OS(O)₂R^{c}, —OS(O)NR^{c}R^{d}, —OS(O)₂NR^{c}R^{d}, —SR^{e}, —S(O)R^{e}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{e}C(O)OR^{d}, —NR^{c}C(O)NR^{c}R^{d}, —NR^{c}C(═NR^{d})NR^{e}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{e}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{e}, —CN, or —NO₂; or R⁷ and one of R³, R⁴, R⁵, or R⁶, taken together with the atoms to which each is attached, combine to form a 4- to 7-membered heterocycloalkyl, wherein each hydrogen atom in 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁸ is H, —S(O)₂R^{e}, —S(O)₂NR^{c}R^{d}, —C(O)R^{c}, —C(O)OR^{c}, —C(O)NR^{e}R^{d}, —C(═NR^{d})NR^{c}R^{d}, —P(O)₂R^{c}R^{d}, —P(O)₂NR^{c}R^{d}, or —P(O)₂OR^{c};
R⁹ is H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{c}, —OC(O)NR^{a}R^{b}, —OS(O)R^{a}, —OS(O)₂R^{a}, —SR^{c}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{e}, —OC(O)NR^{c}R^{d}, —OC(═NR^{d})NR^{c}R^{d}, —OS(O)R^{c}, —OS(O)₂R^{e}, —OS(O)NR^{c}R^{d}, —OS(O)₂NR^{c}R^{d}, —SR^{c}, —S(O)RC, —S(O)₂R, —S(O)NR^{c}R^{d}, —S(O)₂NR^{c}R^{d}, —NR^{c}R^{d}, —NR^{c}C(O)R^{d}, —N(C(O)R^{e})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{e}S(O)₂R^{d}, —NR^{c}S(O)NR^{c}R^{d}, —NR^{c}S(O)₂NR^{c}R^{d}, —C(O)R^{e}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{d}, —P(O)₂OR^{d}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl; or R^{a} and R^{b} or R^{c} and R^{d} or R^{c} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl is independently optionally substituted by deuterium, halogen, C₁-C₆ alkyl, C₁-C₆haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, or 3;
n is 0, 1, 2, or 3;
p is 1, 2, 3, or 4; and
q is 1, 2, or 3;
or a pharmaceutically acceptable salt, solvate, hydrate, or cocrystal thereof.

2. The compound of clause 1, having the formula II

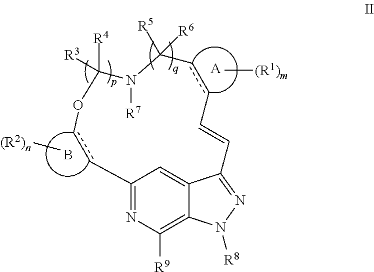

wherein each “ [symbol] ” is independently a carbon-carbon single bond or a carbon-carbon double bond;
or a pharmaceutically acceptable salt, solvate, hydrate, or cocrystal thereof.

3. The compound of clause 1 or 2, having the formula III

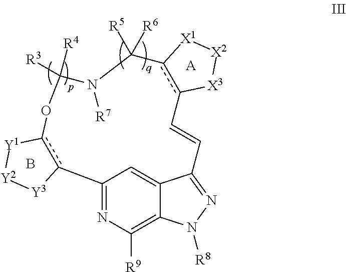

wherein
each “ [symbol] ” is independently a carbon-carbon single bond or a carbon-carbon double bond;
X¹, X², and X³ are each independently —O—, —S—, ═C(H)—, ═C(R¹)—, —N(H)—, —N(R¹)— or ═N—, provided that at least one of X¹, X², and X³ is not ═C(H)—, or ═C(R¹)—; and/or
Y¹, Y², and Y³ are each independently —O—, —S—, ═C(H)—, ═C(R²)—, —N(H)—, —N(R²)— or ═N—, provided that at least one of Y¹, Y², and Y³ is not ═C(H)—, or ═C(R²)—;
or a pharmaceutically acceptable salt, solvate, hydrate, or cocrystal thereof.

4. The compound of clause 3, having the formula IV

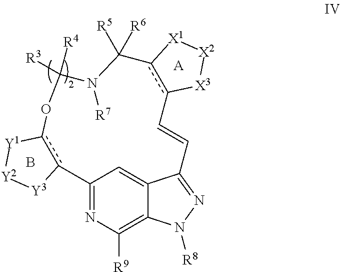

or a pharmaceutically acceptable salt, solvate, hydrate, or cocrystal thereof.

5. The compound of clause 3 or 4, wherein
X² is ═N— or —N(R¹)—, X¹ and X³ are each independently —O—, —S—, ═C(H)—, ═C(R¹)—, —N(H)—, or —N(R¹)—; and/or
Y² is ═N—, Y¹ and Y³ are each independently —O—, —S—, ═C(H)—, ═C(R²)—, —N(H)—, or —N(R²)—;
or a pharmaceutically acceptable salt, solvate, hydrate, or cocrystal thereof.

6. The compound of any one of clauses 1 to 4, wherein:
ring A is selected from the group consisting of

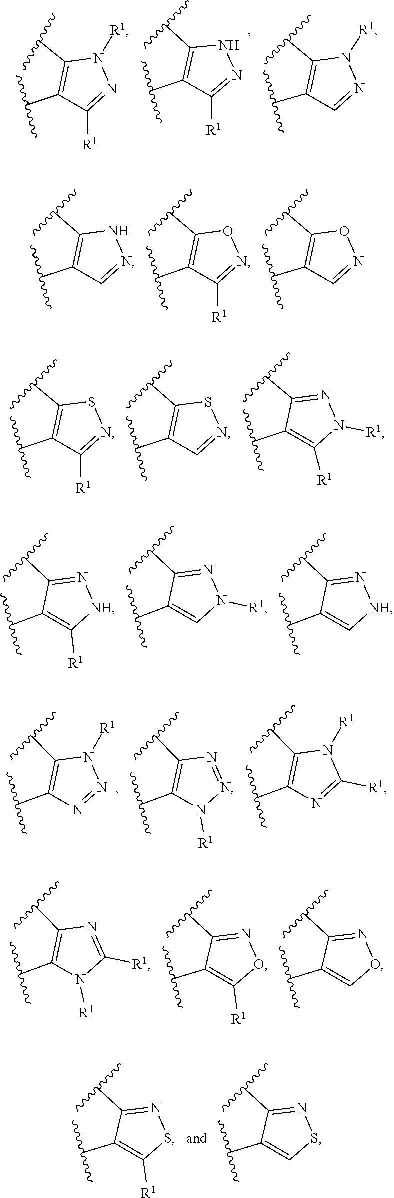

wherein each [symbol] represents a point of covalent attachment;
or a pharmaceutically acceptable salt, solvate, hydrate, or cocrystal thereof.

7. The compound of any one of the preceding clauses, wherein ring A is selected from the group consisting of

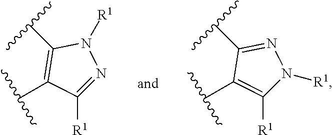

wherein each “ [symbol] ” represents a point of covalent attachment;
or a pharmaceutically acceptable salt, solvate, hydrate, or cocrystal thereof.

8. The compound of any one of the preceding clauses, wherein ring A is selected from the group consisting of

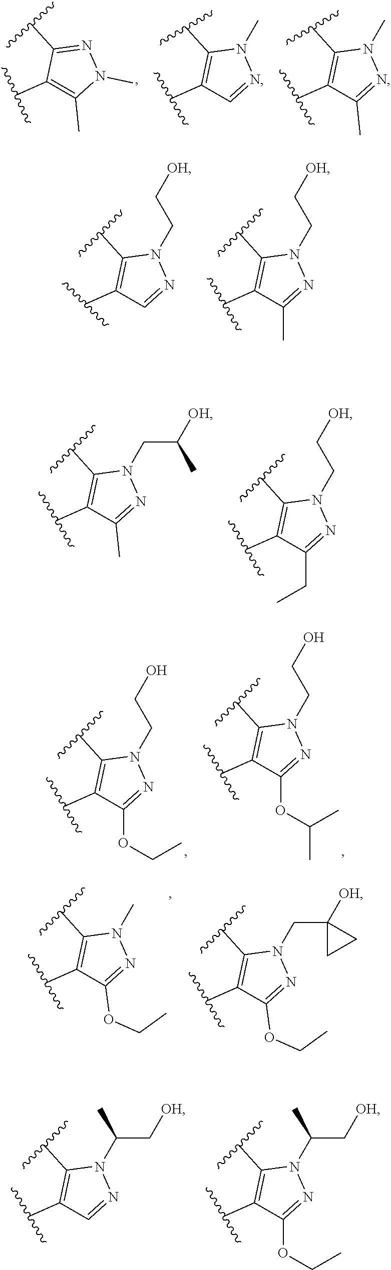

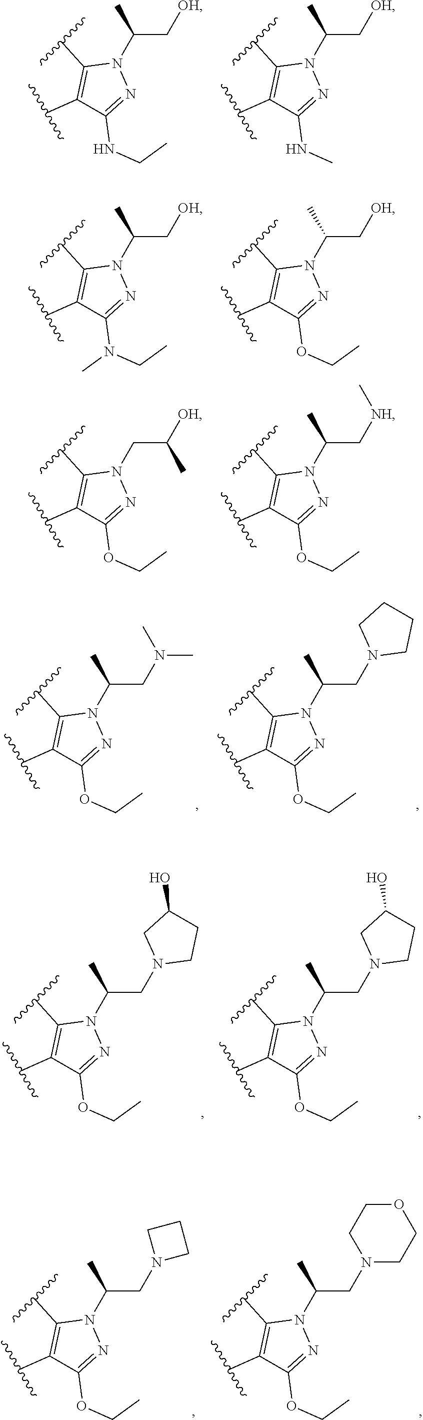

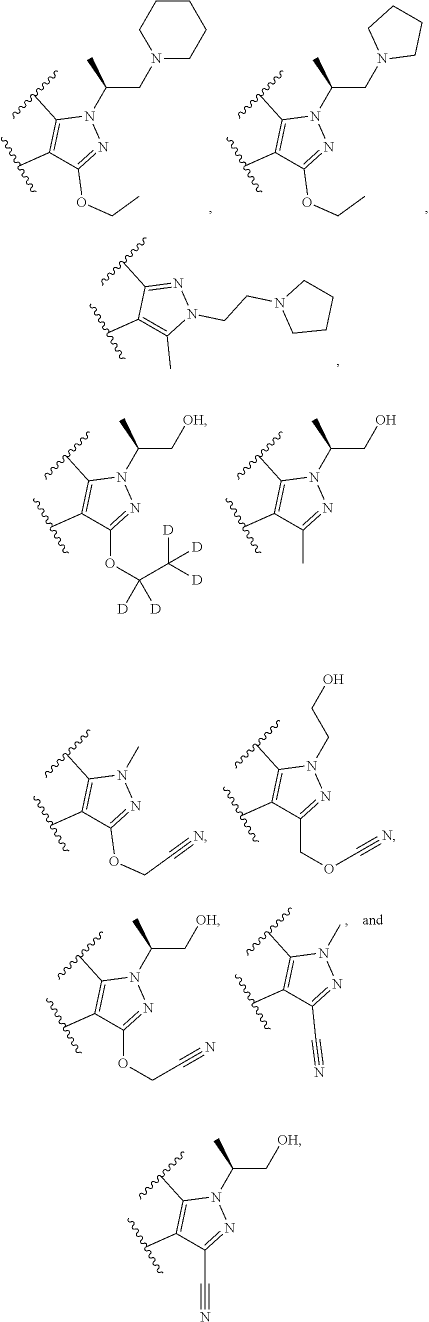

wherein each “ [symbol] ” represents a point of covalent attachment;
or a pharmaceutically acceptable salt, solvate, hydrate, or cocrystal thereof.

9. The compound of any one of clauses 1 to 4 or 6, wherein ring B is selected from the group consisting of

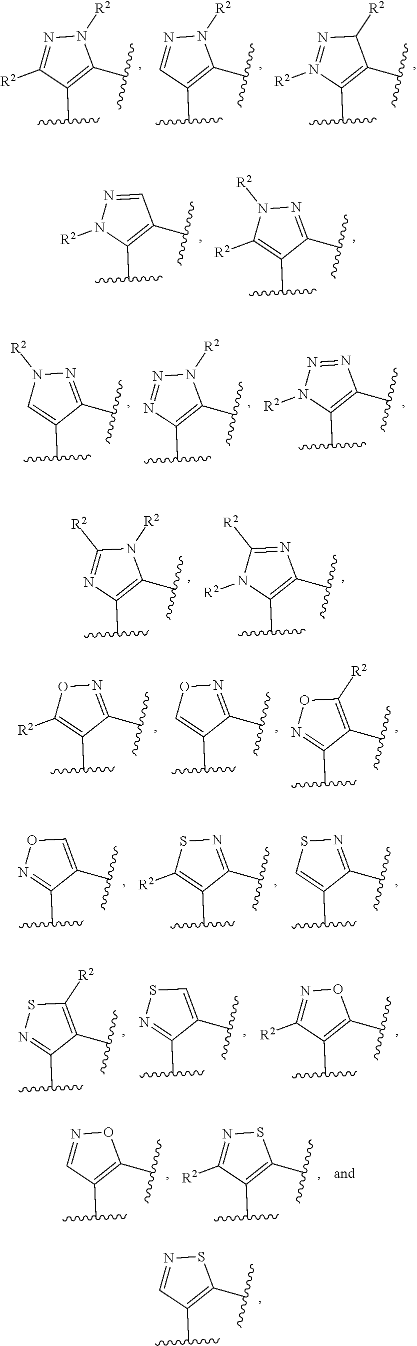

wherein each “ [symbol] ” represents a point of covalent attachment;
or a pharmaceutically acceptable salt, solvate, hydrate, or cocrystal thereof.

10. The compound of any one of the preceding clauses, wherein ring B is selected from the group consisting of

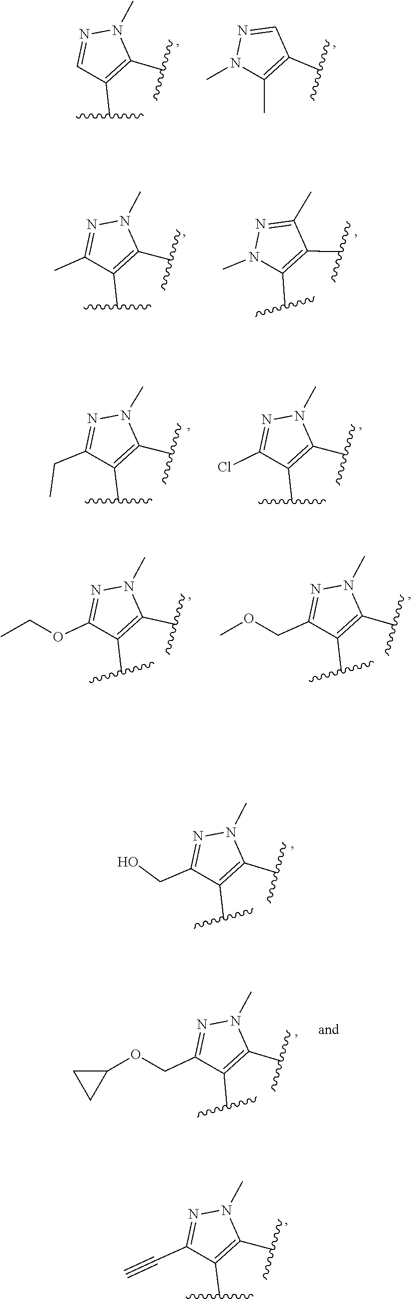

wherein each “ [symbol] ” represents a point of covalent attachment;
or a pharmaceutically acceptable salt, solvate, hydrate, or cocrystal thereof.

11. The compound of any one of the preceding clauses, wherein p is 2 or 3, and the portion

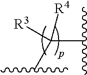

is an ethylene or propylene, wherein each R³ and R⁴ is independently H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or at least one instance of R³ and R⁴, taken together with the carbon or carbons to which they are attached, combine to form C₃-C₆ cycloalkyl or 4- to 7-membered heterocycloalkyl, wherein each hydrogen atom in C₃-C₆ cycloalkyl or 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R⁷ and one instance of R³ or R⁴, taken together with the atoms to which they are attached, combine to form 4- to 7-membered heterocycloalkyl, wherein each hydrogen atom in 4- to 7-membered heterocycloalkyl formed is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{c}, —S(O)NR^{e}R^{f}, —S(O)₂NR^{e}R^{f}, —NR^{e}R^{f}, —NR^{c}C(O)R^{f}, —NR^{c}C(O)OR^{f}, —NRC(O)NR^{r}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and each “ [symbol] ” represents a point of covalent attachment;
or a pharmaceutically acceptable salt, solvate, hydrate, or cocrystal thereof.

12. The compound of any one of the preceding clauses, wherein one R³ is C₁-C₆ alkyl, wherein each hydrogen atom in C₁-C₆ alkyl is independently optionally substituted by deuterium, halogen, C₁-C₆ alkyl, C₁-C₆haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or two R³ and R⁴, taken together with the carbon or carbons to which they are attached, combine to form C₃-C₆ cycloalkyl or 4- to 7-membered heterocycloalkyl, wherein each hydrogen atom in C₃-C₆ cycloalkyl and 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or R⁷ and one instance of R³ or R⁴ and, taken together with the atoms to which they are attached, combine to form a 4- to 7-membered heterocycloalkyl, wherein each hydrogen atom in 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and any remaining R³ and R⁴ are H or deuterium;
or a pharmaceutically acceptable salt, solvate, hydrate, or cocrystal thereof.

13. The compound of any one of the preceding clauses, wherein one R³ is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{c}S(O)R^{f}, —NR^{c}S(O)₂R^{f}, —NR^{c}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or R⁷ and one instance of R³ or R⁴ taken together with the atoms to which they are attached, combine to form 4- to 7-membered heterocycloalkyl, wherein each hydrogen atom in 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{c}, —P(O)₂OR^{e}, —CN, or —NO₂; and any remaining R³ and R⁴ are H or deuterium;
or a pharmaceutically acceptable salt, solvate, hydrate, or cocrystal thereof.

14. The compound of any one of the preceding clauses, wherein one instance of R³ is C₁-C₆ alkyl; and any remaining R³ and R⁴ are H or deuterium;
or a pharmaceutically acceptable salt, solvate, hydrate, or cocrystal thereof.

15. The compound of any one of the preceding clauses, wherein one instance of R³ is C₁-C₆ alkyl; R⁷ and one instance of R⁴, taken together with the atoms to which they are attached, combine to form a 4- to 7-membered heterocycloalkyl; and any remaining R³ and R⁴ are H or deuterium;
or a pharmaceutically acceptable salt, solvate, hydrate, or cocrystal thereof.

16. The compound of any one of the preceding clauses, wherein one instance of R³ is methyl, and any remaining R³ and R⁴ are H or deuterium;
or a pharmaceutically acceptable salt, solvate, hydrate, or cocrystal thereof.

17. The compound of any one of the preceding clauses, wherein R⁷ is H, C₁-C₆ alkyl, C₃-C₆ cycloalkyl, or —C(O)R^{c}; or R⁷ and one instance of R³ or R⁴, taken together with the atoms to which they are attached, combine to form 4- to 7-membered heterocycloalkyl; wherein each hydrogen atom in C₁-C₆ alkyl, C₃-C₆ cycloalkyl, and 4- to 7-membered heterocycloalkyl is independently optionally substituted by —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{d}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
or a pharmaceutically acceptable salt, solvate, hydrate, or cocrystal thereof.

18. The compound of any one of the preceding clauses, wherein the portion

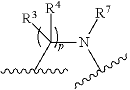

is of the formula

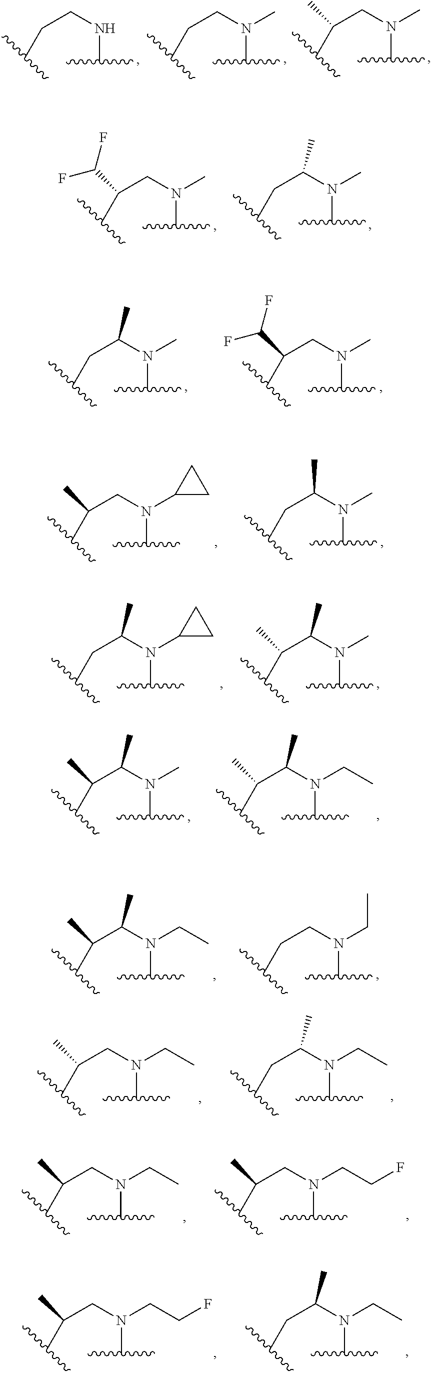

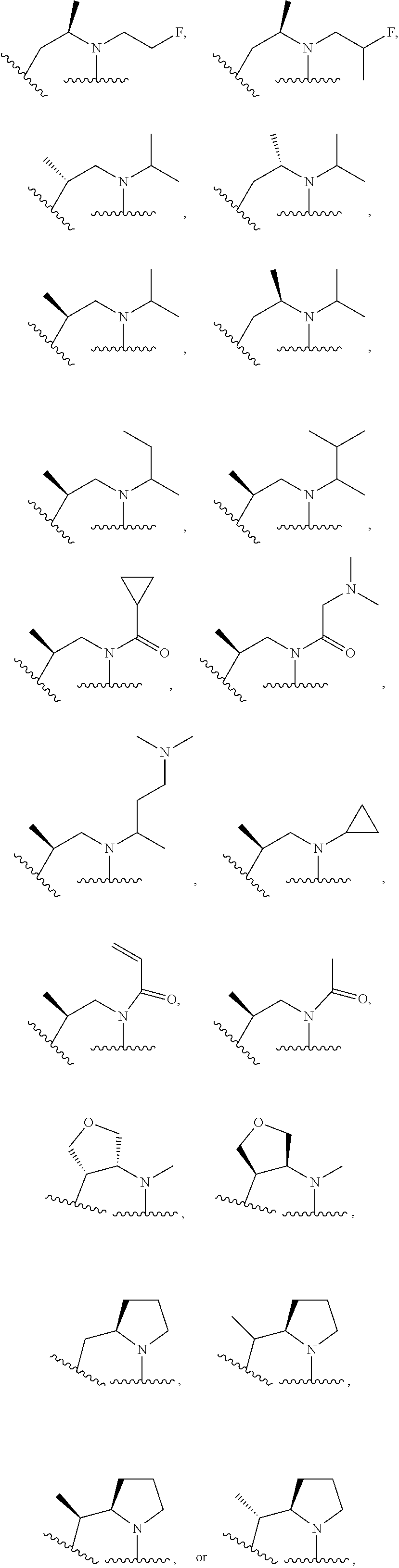

wherein each “ [symbol] ” represents a point of covalent attachment, and each hydrogen is independently optionally substituted with deuterium;
or a pharmaceutically acceptable salt, solvate, hydrate, or cocrystal thereof.

19. The compound of any one of the preceding clauses, wherein R⁵ and R⁶ are each H;
or a pharmaceutically acceptable salt, solvate, hydrate, or cocrystal thereof.

20. The compound of any one of the preceding clauses, wherein the portion

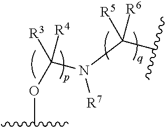

is of the formula

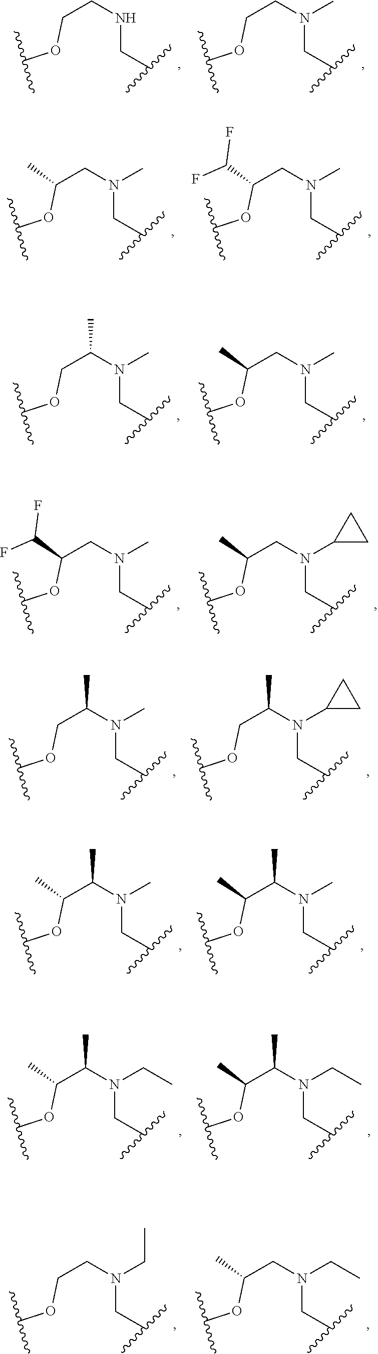

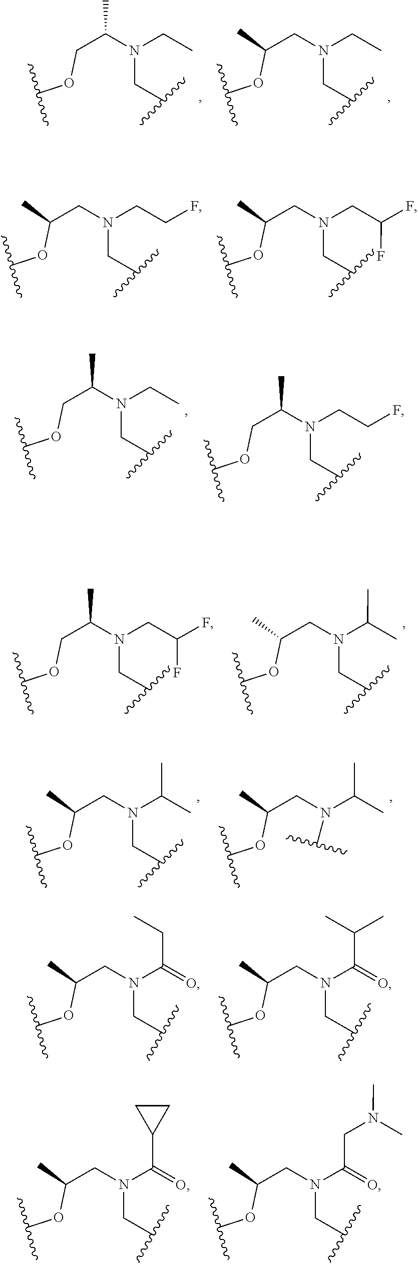

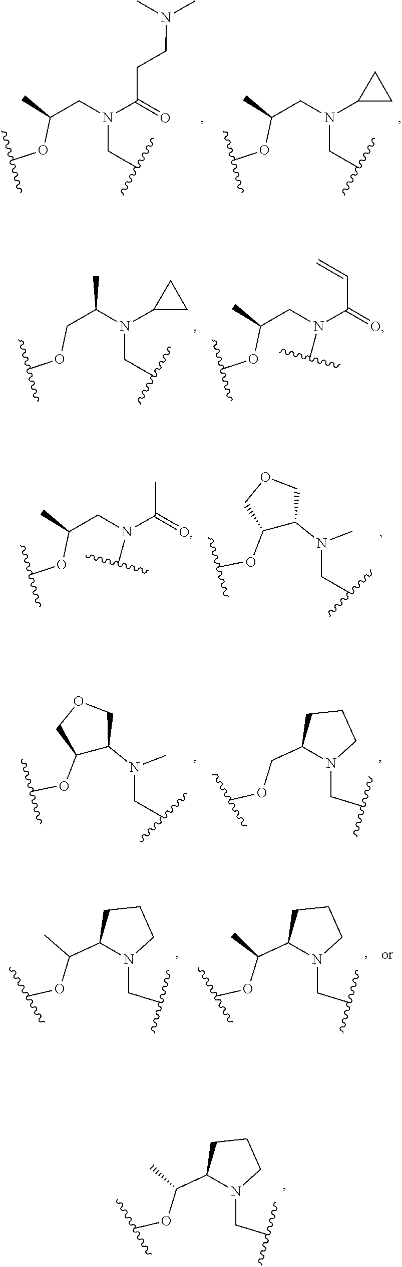

wherein each “ [symbol] ” represents a point of covalent attachment, and each hydrogen is independently optionally substituted with deuterium;
or a pharmaceutically acceptable salt, solvate, hydrate, or cocrystal thereof.

21. The compound of any one of the preceding clauses, wherein R⁸ is H or C₁-C₆ alkyl;
or a pharmaceutically acceptable salt, solvate, hydrate, or cocrystal thereof.

22. The compound of any one of the preceding clauses, wherein R⁹ is H, deuterium, halogen, C₁-C₆ alkyl, or —OR^{a}, wherein each hydrogen atom in C₁-C₆ alkyl is independently optionally substituted with deuterium;
or a pharmaceutically acceptable salt, solvate, hydrate, or cocrystal thereof.

23. The compound of any one of the preceding clauses, wherein R⁹ is H, deuterium, halogen, —OCH₃, —CH₃, or —CD₃;
or a pharmaceutically acceptable salt, solvate, hydrate, or cocrystal thereof.

24. The compound of clause 1, selected from the group consisting of

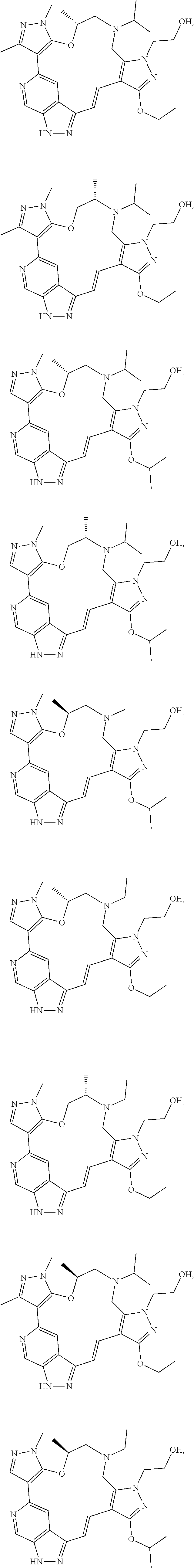

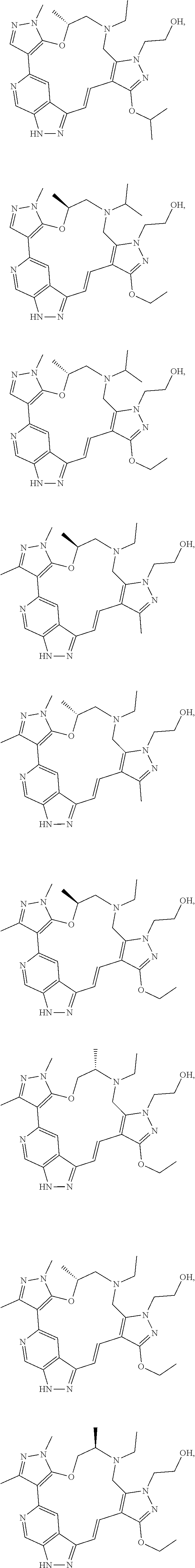

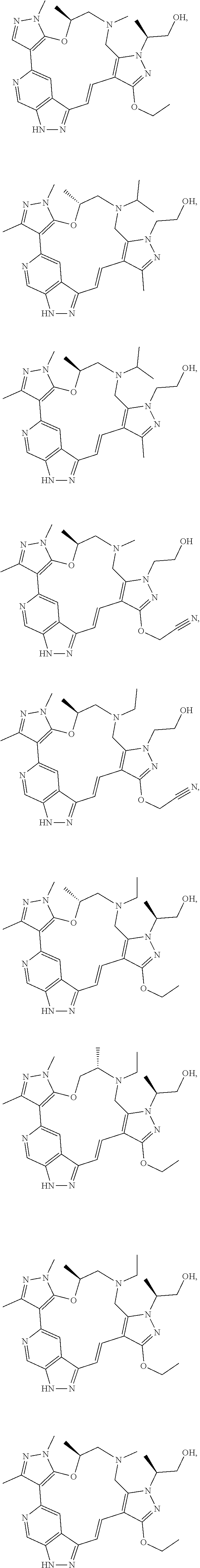

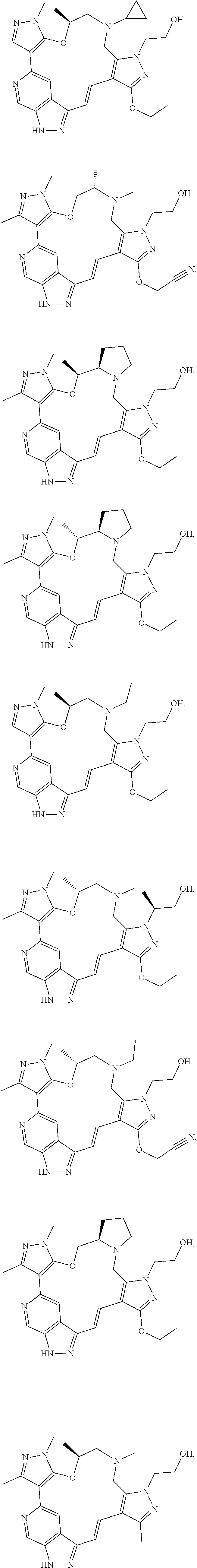

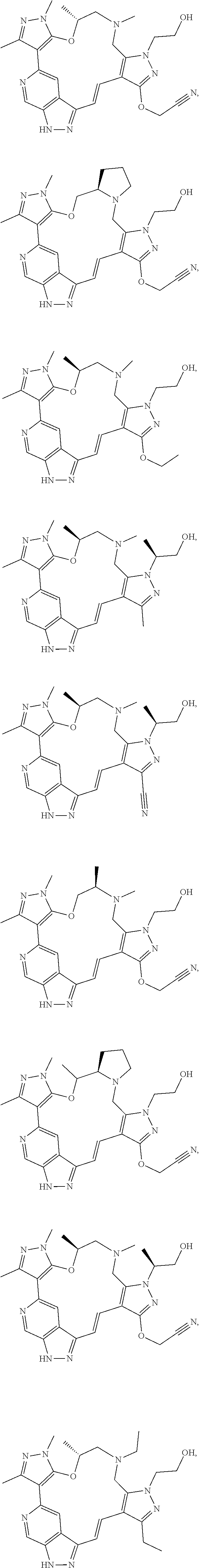

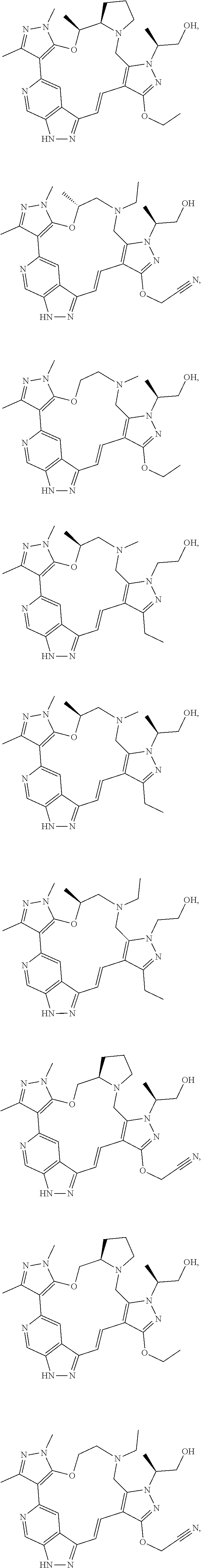

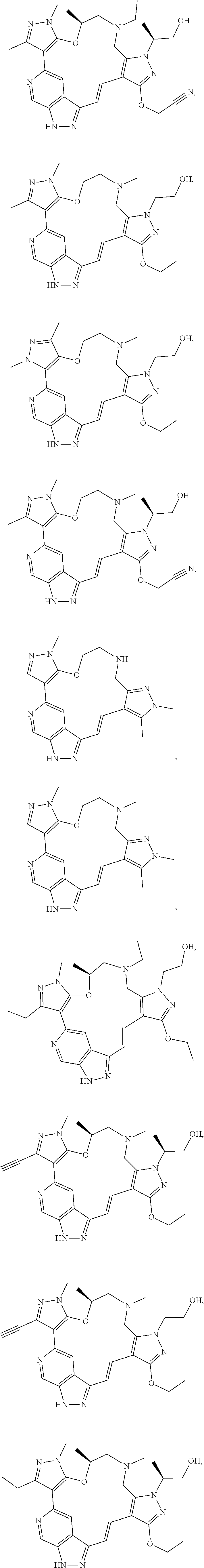

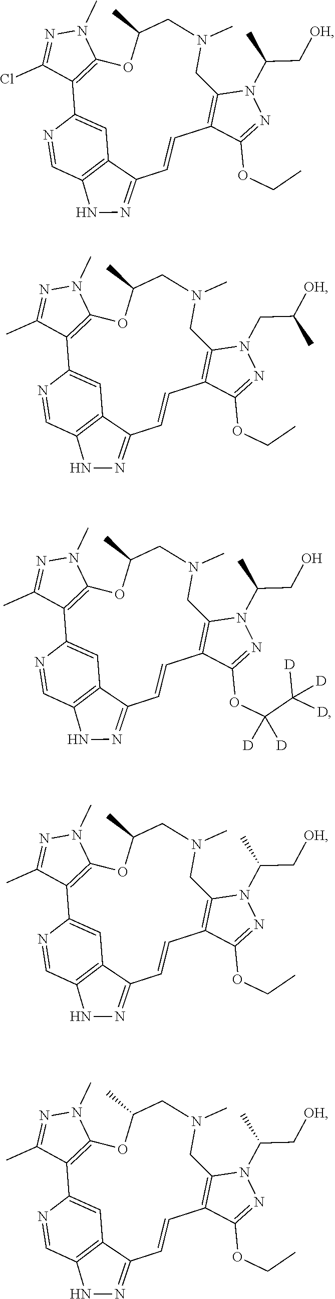

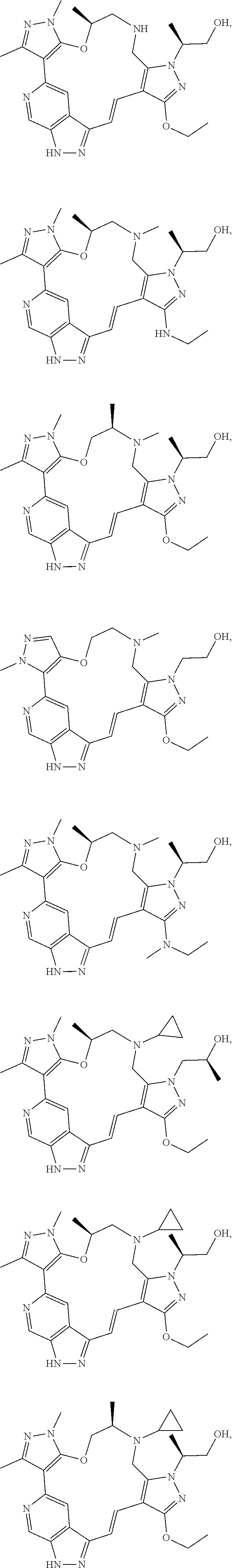

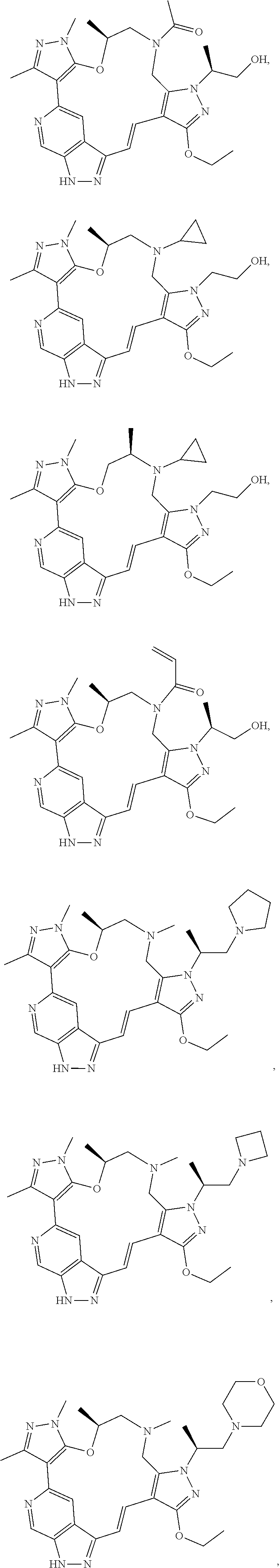

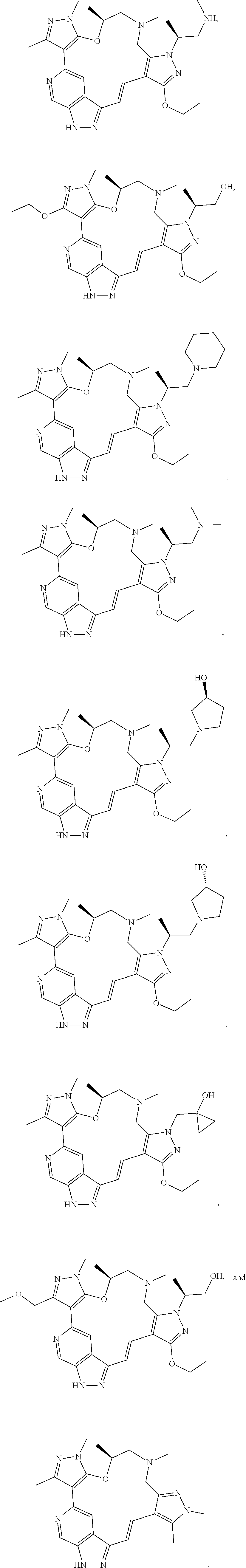

or a pharmaceutically acceptable salt, solvate, hydrate, or cocrystal thereof.

25. The compound of clause 1, selected from the group consisting of

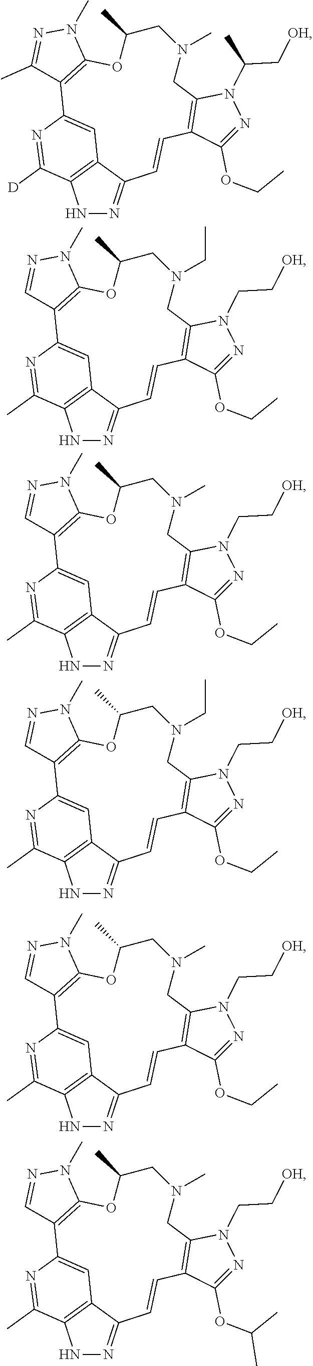

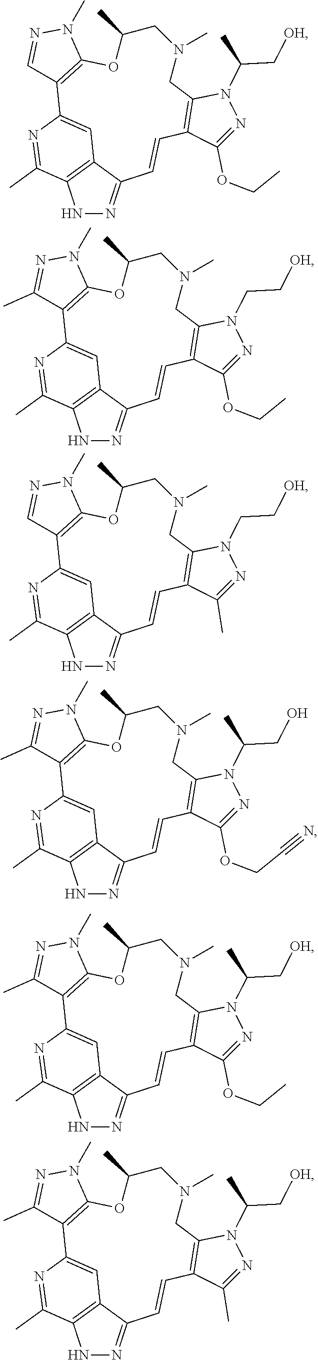

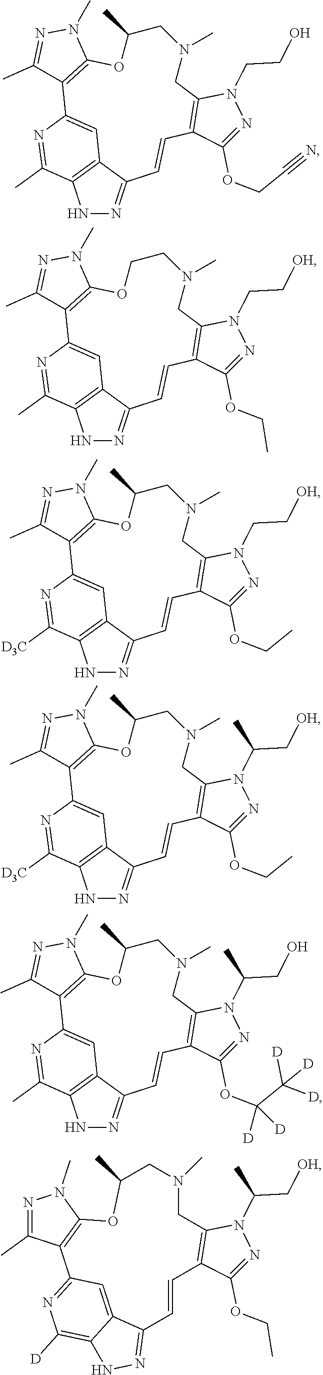

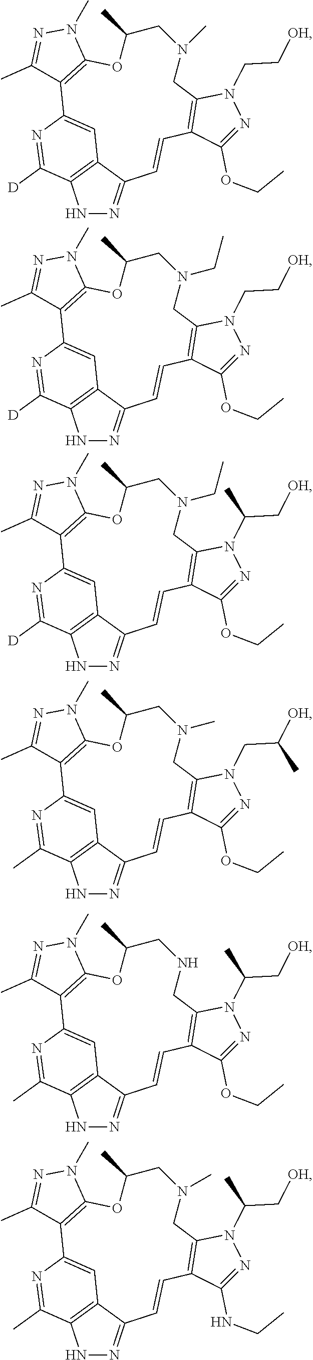

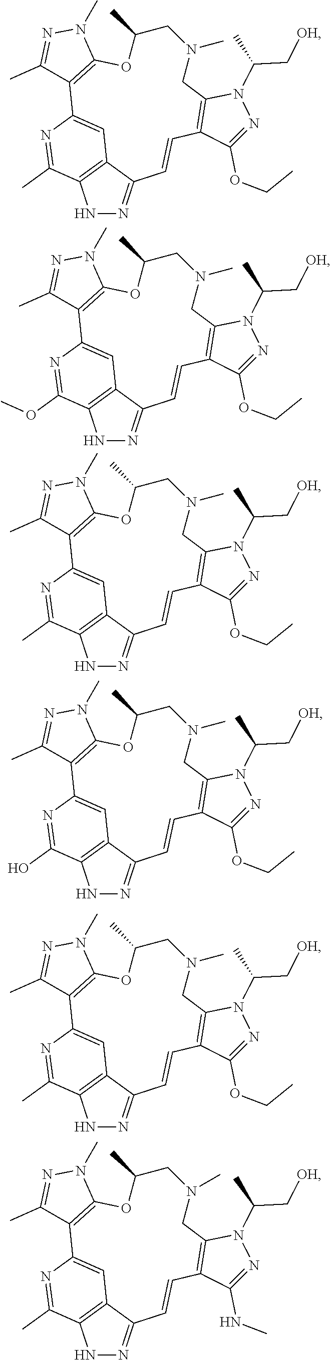

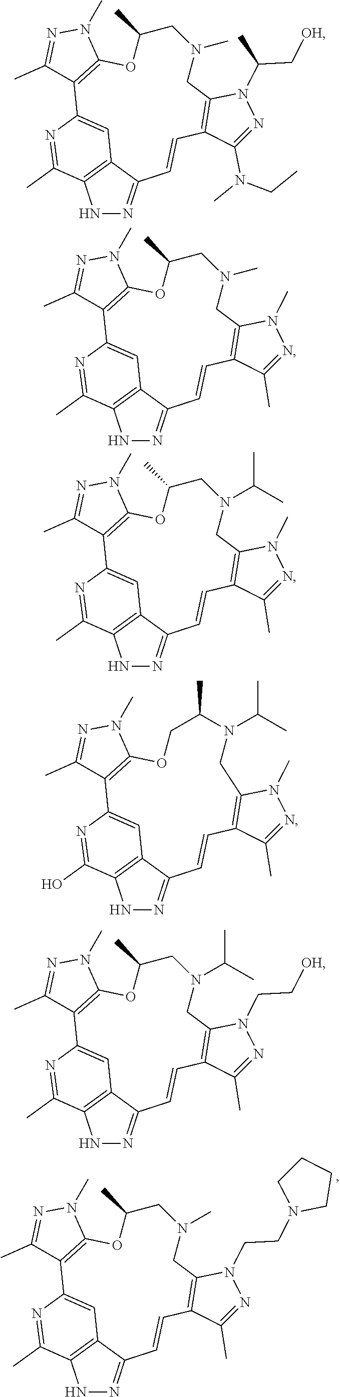

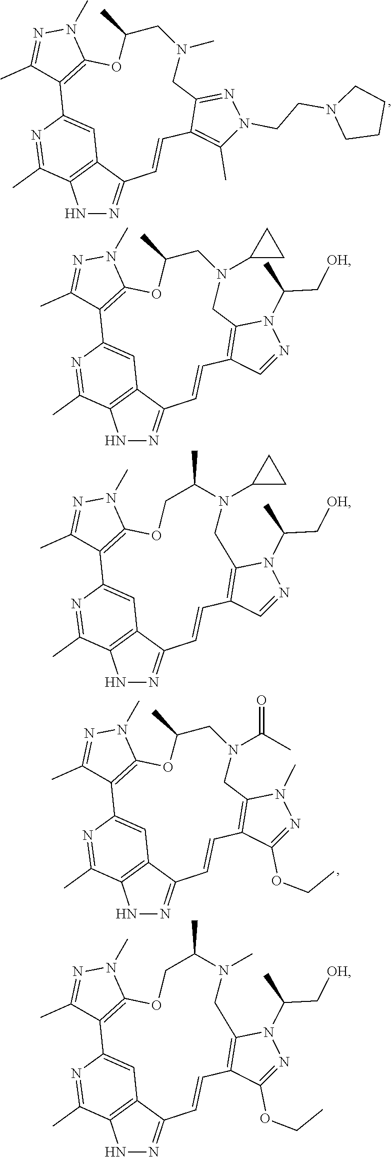

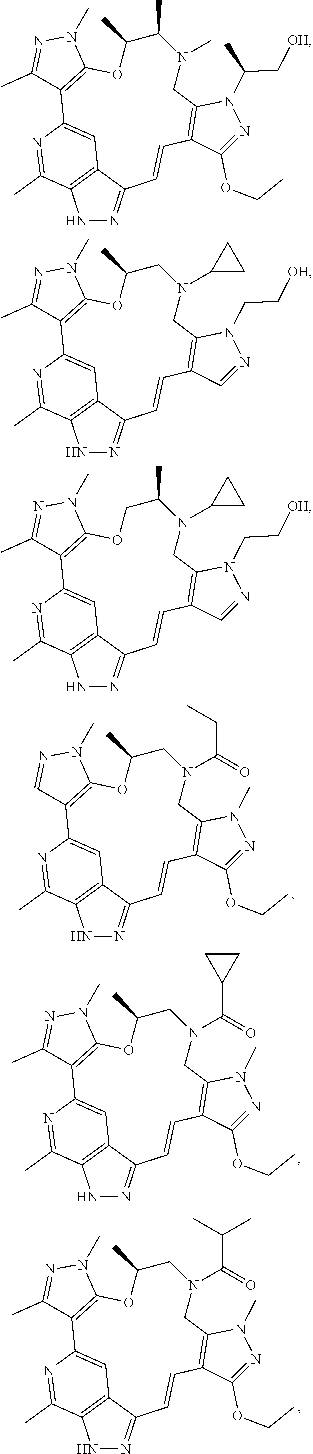

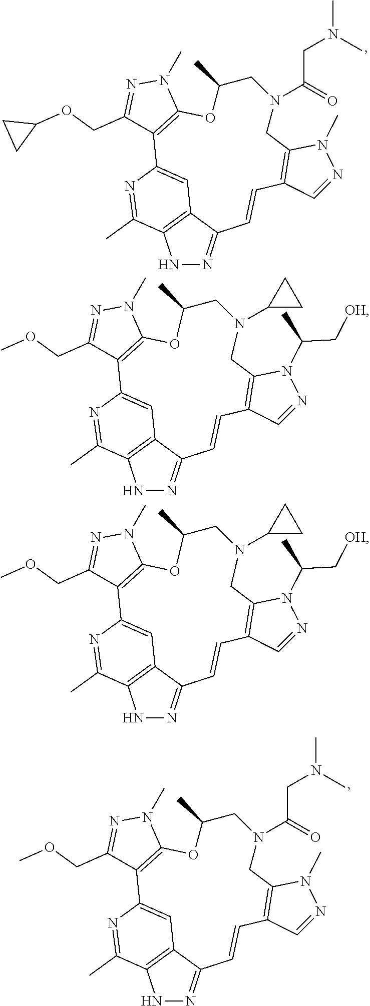

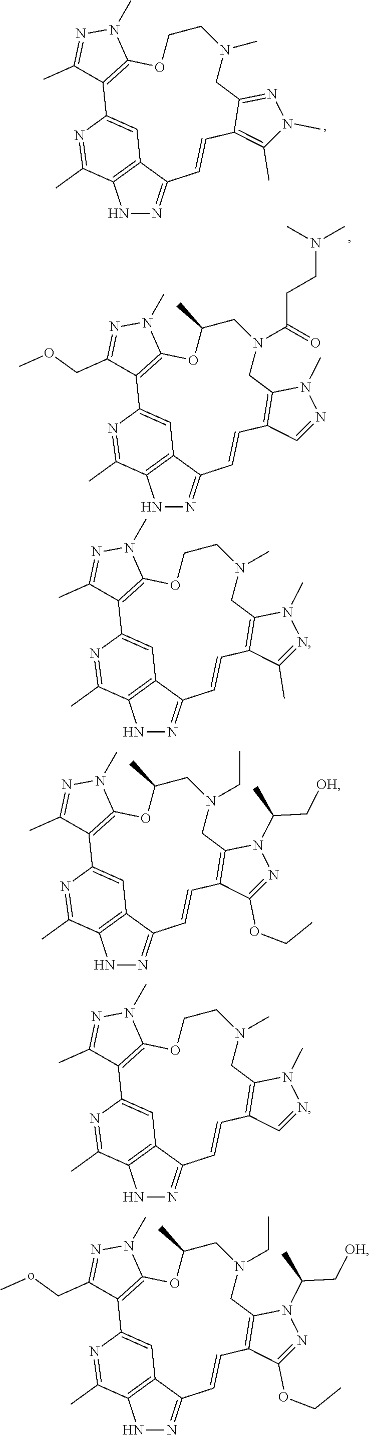

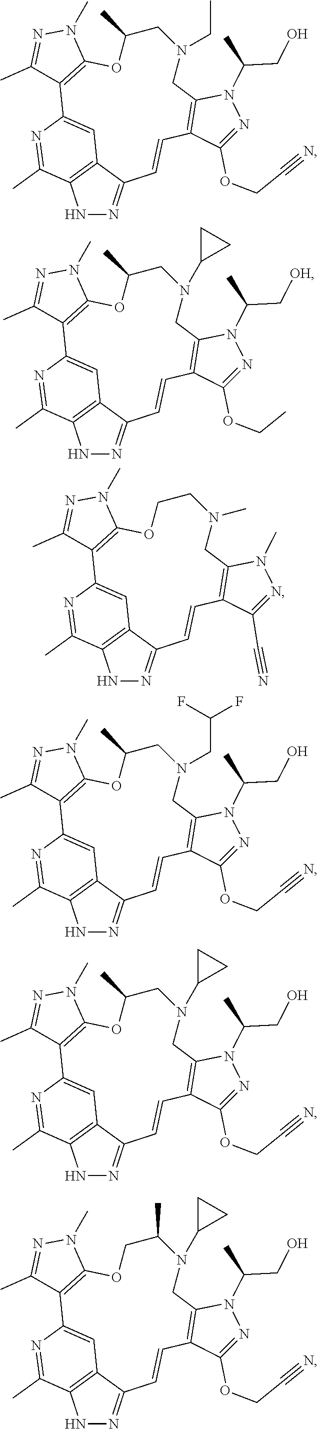

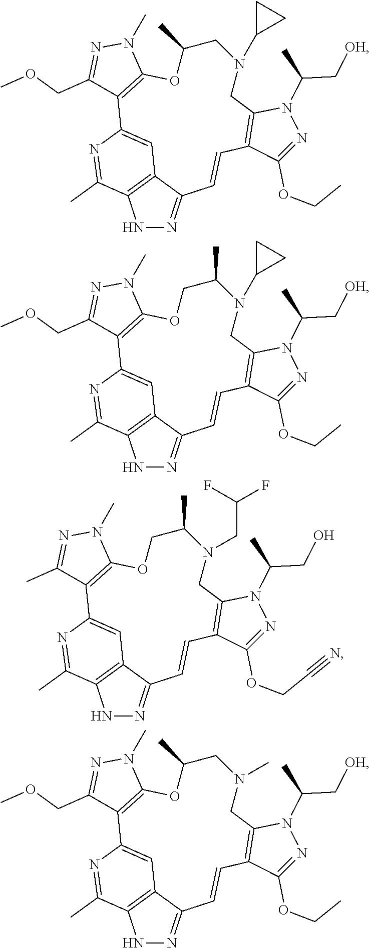

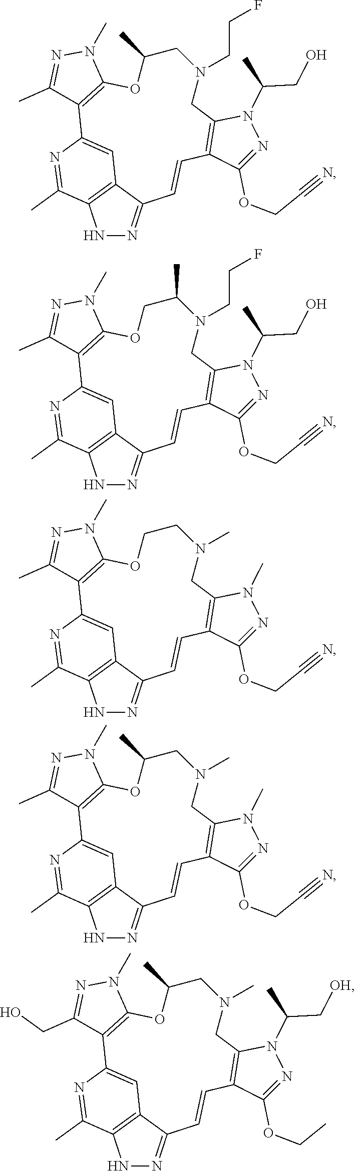

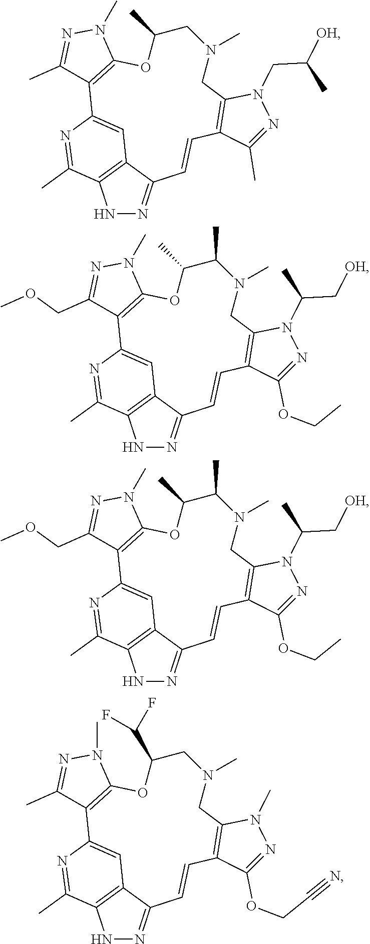

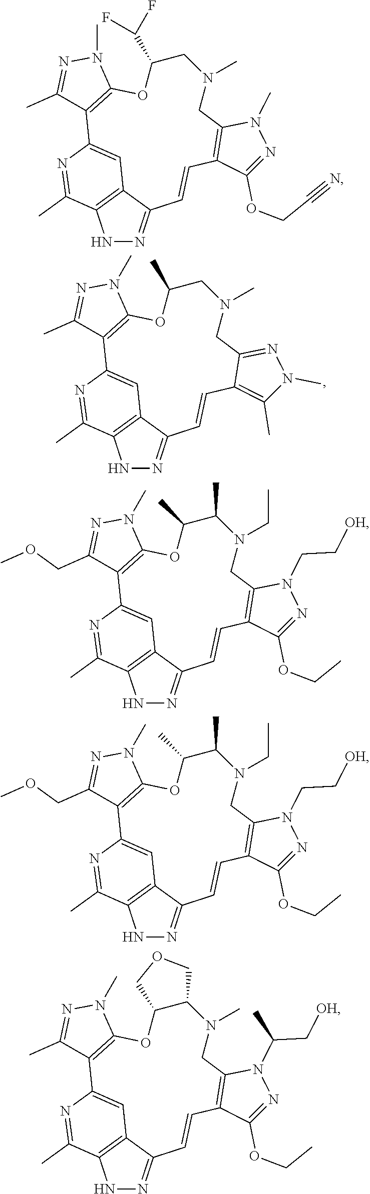

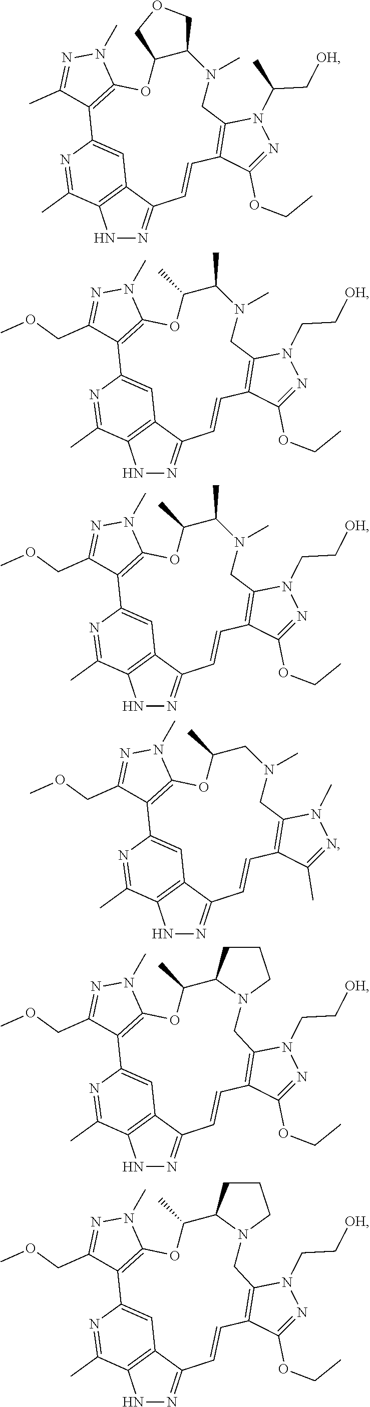

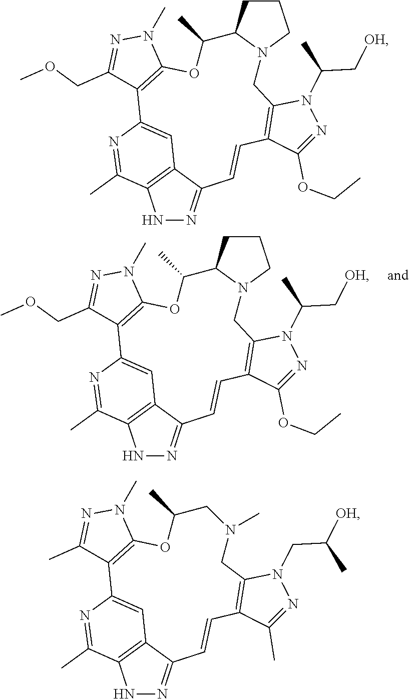

or a pharmaceutically acceptable salt, solvate, hydrate, or cocrystal thereof.

26. A pharmaceutical composition comprising a compound of any one of the preceding clauses, or a pharmaceutically acceptable salt, solvate, hydrate, or cocrystal thereof, and optionally one or more excipients.

27. A method of treating disease in a subject comprising, administering a therapeutically effective amount of a compound of any one of clauses 1 to 25, or a pharmaceutically acceptable salt, solvate, hydrate, or cocrystal thereof, or a pharmaceutical composition of clause 26.

28. A compound according to any one of clauses 1 to 25, or a pharmaceutically acceptable salt, solvate, hydrate, or cocrystal thereof, for use in a method of treating disease in a subject.

29. Use of a compound according to any one of clauses 1 to 25, or a pharmaceutically acceptable salt, solvate, hydrate, or cocrystal thereof, in the manufacture of a medicament for the treatment of disease in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-**” “A-**” or

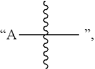

where each of “-*”, “-**”, and

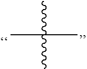

represents a bond to A and the point of covalent bond attachment to B. Alternatively, in some embodiments, the portion of A-B defined by the group or chemical structure B can be represented by “*-B” “**-B”, or

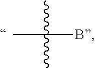

where each of “-*”, “-**”, and

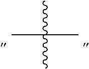

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “alkynyl” refers to a straight- or branched-chain monovalent hydrocarbon group having one or more triple bonds. In some embodiments, it can be advantageous to limit the number of atoms in an “alkynyl” to a specific range of atoms, such as C₂-C₂₀ alkynyl, C₂-C₁₂ alkynyl, or C₂-C₆ alkynyl. Examples of alkynyl groups include acetylenyl (—C≡CH) and propargyl (—CH₂C≡CH), but-3-yn-1,4-diyl (—C≡C—CH₂CH₂—), and the like. It will be appreciated that an alkynyl group can be unsubstituted or substituted as described herein. An alkynyl group can be substituted with any of the substituents in the various embodiments described herein, including one or more of such substituents.

The term “cycloalkyl” refers to a saturated or partially saturated, monocyclic or polycyclic mono-valent carbocycle. In some embodiments, it can be advantageous to limit the number of atoms in a “cycloalkyl” to a specific range of atoms, such as having 3 to 12 ring atoms. Polycyclic carbocycles include fused, bridged, and spiro polycyclic systems. Illustrative examples of cycloalkyl groups include monovalent radicals of the following entities:

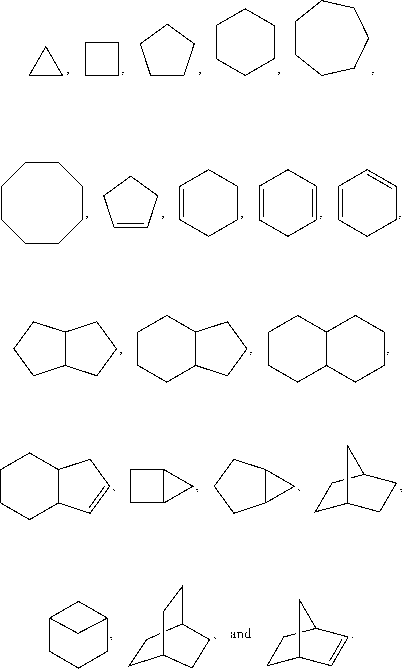

In particular, a cyclopropyl moiety can be depicted by the structural formula

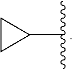 [SMILES: *C1CC1]

It will be appreciated that a cycloalkyl group can be unsubstituted or substituted as described herein. A cycloalkyl group can be substituted with any of the substituents in the various embodiments described herein, including one or more of such substituents.

The term “oxo” represents a carbonyl oxygen.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F. The term “aryl” refers to a monovalent all-carbon monocyclic or fused-ring polycyclic group having a completely conjugated pi-electron system. In some embodiments, it can be advantageous to limit the number of atoms in an “aryl” to a specific range of atoms, such as mono-valent all-carbon monocyclic or fused-ring polycyclic groups of 6 to 14 carbon atoms (C₆-C₁₄ aryl), or monovalent all-carbon monocyclic or fused-ring polycyclic groups of 6 to 10 carbon atoms (C₆-C₁₀ aryl). Examples, without limitation, of aryl groups are phenyl, naphthalenyl and anthracenyl. It will be appreciated that an aryl group can be unsubstituted or substituted as described herein. An aryl group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. In some embodiments, it can be advantageous to limit the number of atoms in a “heterocycloalkyl” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), 5 to 7 ring atoms (5- to 7-membered), or 4 to 10 ring atoms (4- to 10-membered). In some embodiments, it can be advantageous to limit the number and type of ring heteroatoms in “heterocycloalkyl” or to a specific range or type of heteroatoms, such as 1 to 5 ring heteroatoms selected from nitrogen, oxygen, and sulfur. Examples, without limitations, of mono-cyclic heterocycloalkyl groups include tetrahydrofuran, pyrrolidine, and morpholine. Polycyclic ring systems include fused, bridged, and spiro systems. In some embodiments, it can be advantageous to limit the number of atoms in a bicyclic “heterocycloalkyl” to a specific range of ring atoms, such as from 5 to 10 ring atoms (5- to 10-membered), or 6 to 10 ring atoms (6- to 10-membered). The ring structure may optionally contain an oxo group or an imino group on a carbon ring member or up to two oxo groups on sulfur ring members. Examples, without limitations, of fused bicyclic, bridged bicyclic, and spiro bicyclic heterocycloalkyl groups include pyrrolizine, 2,5-diazabicyclo[2.2.2]octane, and 1-oxaspiro[4.5]decane. Illustrative examples of heterocycloalkyl groups include monovalent radicals of the following entities:

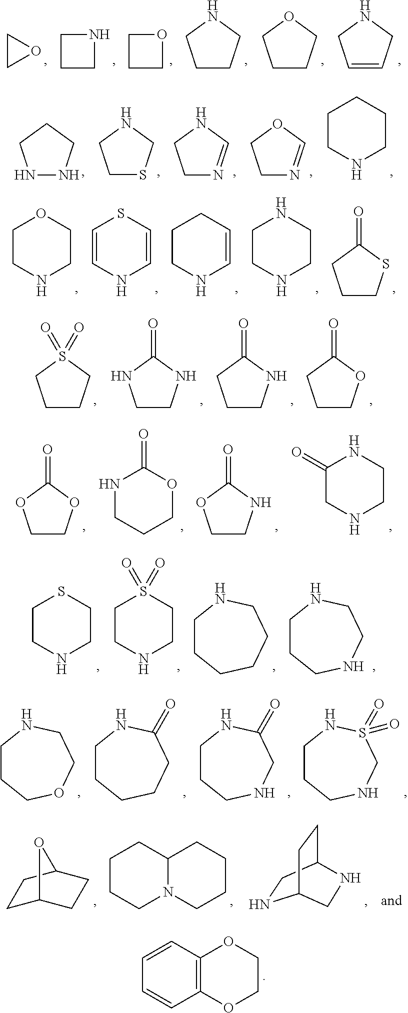

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2, 5-dihydro-1H-pyrrole, pyrazolidine, thiazolidine, 4,5-dihydro-1H-imidazole, dihydrothiophen-2(3H)-one, tetrahydrothiophene 1,1-dioxide, imidazolidin-2-one, pyrrolidin-2-one, dihydrofuran-2(3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

As described herein, certain embodiments can include a heteroatom-C₂-C₆ alkylene moiety (e.g., —N(C₂-C₆ alkylene) or —P(O)₂(C₂-C₆ alkylene)). Illustratively, -heteroatom-(C₂-C₆ alkylene) represents a cyclic group wherein the heteroatom atom (e.g., nitrogen, phosphorus, etc.) forms two covalent bonds with the C₂-C₆ alkylene group

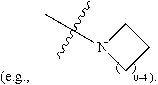

For example, —OC(O)N(C₂-C₆ alkylene) can be depicted by the structural formula

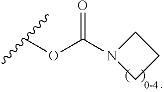

It will be appreciated that a heterocycloalkyl group can be unsubstituted or substituted as described herein. A heterocycloalkyl group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a divalent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl or 5- to 10-membered heteroarylene. In some instances, a 5- to 10-membered heteroaryl can be a monocyclic ring or fused bicyclic rings having 5- to 10-ring atoms wherein at least one ring atom is a heteroatom, such as N, O, or S. In some instances, a 5- to 10-membered heteroarylene can be a monocyclic ring or fused bicyclic rings having 5-to 10-ring atoms wherein at least one ring atom is a heteroatom, such as N, O, or S. The ring structure may optionally contain an oxo group or an imino group on a carbon ring member or up to two oxo groups on sulfur ring members. Illustrative examples of 5- to 10-membered heteroaryl groups include monovalent radicals of the following entities, while examples of 5-to 10-membered heteroarylene groups include divalent radicals of the following entities, in the form of properly bonded moieties:

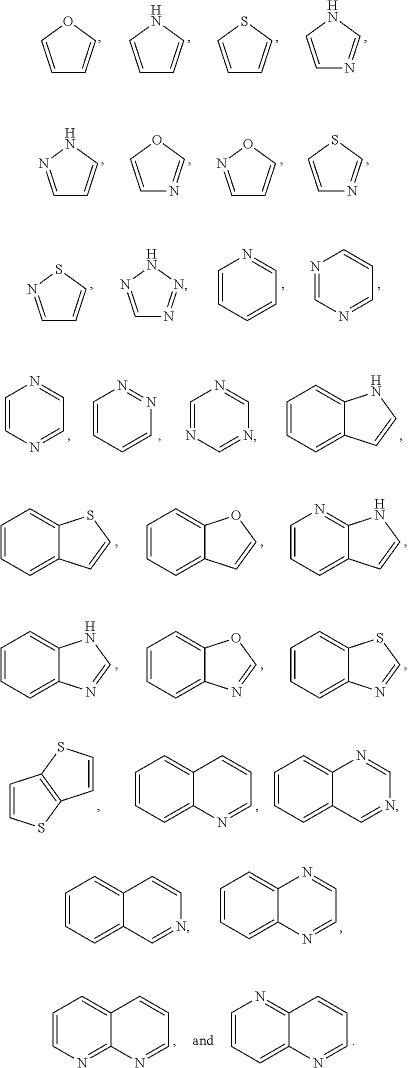

In some embodiments, a “monocyclic” heteroaryl can be an aromatic five- or six-membered heterocycle. A five-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of five-membered heteroarylene groups include di-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. Non-limiting examples of six-membered heteroarylene groups include divalent radicals of pyridine, pyrazine, pyrimidine, pyridazine, or triazine. A “bicyclic heteroaryl” or “bicyclic heteroarylene” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole. Non-limiting examples of bicyclic heteroarylene groups include divalent radicals of azaindazole, indazole, quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole.

In particular, a pyrazolyl moiety can be depicted by the structural formula

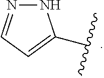 [SMILES: *c1ccn[nH]1]

In particular, an example of a pyrazolylene moiety can be depicted by the structural formula

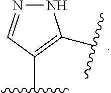 [SMILES: *c1cn[nH]c1*]

In particular, an azaindazolylene or pyrazolopyridylene moiety such as 1H-pyrazolo[3,4-c]pyridylene moiety can be depicted by the structural formula

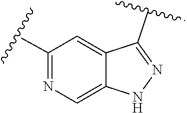

It will be appreciated that a heteroaryl group can be unsubstituted or substituted as described herein. A heteroaryl group can be substituted with any of the substituents in the various embodiments described herein, including one or more of such substituents.

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

As used herein, the phrase “taken together with the atoms to which each is attached” means that two substituents (e.g., R⁷ and one of R³, R⁴, R⁵, or R⁶) attached to two separate atoms combine to form a 4- to 7-membered heterocycloalkyl that are defined by the claim, such as

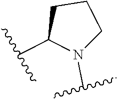 [SMILES: *[C@H]1CCCN1*]

In particular, the phrase “taken together with the atoms to which each is attached, combine to form a 4- to 7-membered heterocycloalkyl” means R⁷ and one of R³, R⁴, R⁵, or R⁶, for example, on different ring atoms of

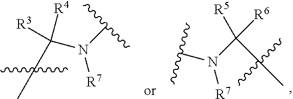

form a 4- to 7-membered ring with those ring atoms.

For example, the phrase “taken together with the atoms to which each is attached, combine to form a 4- to 7-membered heterocycloalkyl” used in connection with the embodiments described herein includes the compound represented as follows:

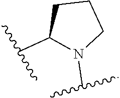 [SMILES: *[C@H]1CCCN1*]

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹C, ¹³C, ¹⁴C, ¹⁵N, ¹⁸O, ¹⁷O, ³¹P, ³³P, ³⁵, ¹⁸F, ³⁶Cl, and ¹²⁵I, respectively. Such isotopically labelled compounds are useful in metabolic studies (preferably with ¹⁴C), reaction kinetic studies (with, for example ²H or 3H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes pharmaceutically acceptable salts of the compounds represented by Formula (I)-(IV), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)-(IV) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)-(IV), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)-(IV). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs,” ed. H. Bundgaard, Elsevier, 1985.

The present disclosure also relates to pharmaceutically active metabolites of compounds of Formula (I)-(IV), and uses of such metabolites in the methods of the disclosure. A “pharmaceutically active metabolite” means a pharmacologically active product of metabolism in the body of a compound of Formula (I)-(IV) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

As used herein, the term “EGFR inhibitor” includes, but is not limited to, a compound that is capable of inhibiting the protein encoded by the EGFR gene. EGFR inhibitors include, but are not limited to compounds that are capable of inhibiting the protein that is encoded by the classical EGFR mutations, uncommon mutations, and secondary resistance mutations. Examples of EGFR mutations include, but are not limited to L858R, Δ746-750, Δ746-750/C979S, L858R/T790M, L858R/T790M/C979S, and D770_N771insNPG, other emerging and established resistance mutations, and the like. It will be understood by a person having ordinary skill in the art that reference to a inhibiting of EGFR mutations, such as EGFR L858R refers to inhibiting the protein encoded by a single missense mutation in exon 21 of the EGFR gene.

### Representative Embodiments

In some embodiments, the disclosure provides a compound of the formula I, or a pharmaceutically acceptable salt, solvate, hydrate, or cocrystal thereof

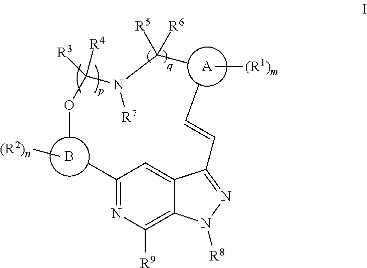

wherein R¹, R², R³, R⁴, R⁵, R⁶, R⁷, R⁸, R⁹, A, B, m, n, p, and q are as described herein.

In some embodiments, the disclosure relates to a compound of the formula II, or a pharmaceutically acceptable salt, solvate, hydrate, or cocrystal thereof,

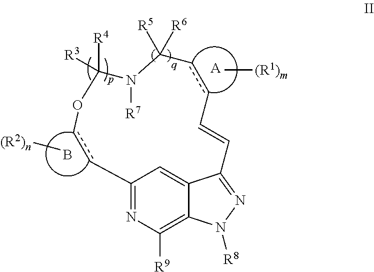

wherein R¹, R², R³, R⁴, R¹, R⁶, R⁷, R⁸, R⁹, A, B, m, n, p, and q are as described herein.

In some embodiments, the disclosure relates to a compound of the formula III, or a pharmaceutically acceptable salt, solvate, hydrate, or cocrystal thereof,

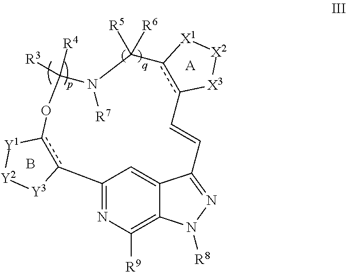

wherein X¹, X², X³, Y¹, Y², Y³, R³, R⁴, R¹, R⁶, R⁷, R⁸, R⁹, A, B, p, and q are as described herein.

In some embodiments, the disclosure relates to a compound of the formula IV, or a pharmaceutically acceptable salt, solvate, hydrate, or cocrystal thereof,

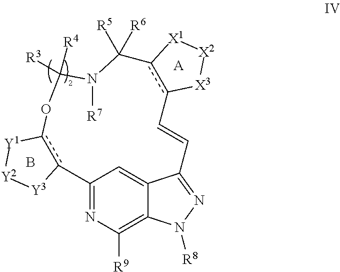

wherein X¹, X², X³, Y¹, Y², Y³, R³, R⁴, R⁵, R⁶, R⁷, R⁸, R⁹, A, and B are as described herein.

In some embodiments, X¹, X², and X³ are each independently —O—, —S—, ═C(H)—, ═C(R¹)—, —N(H)—, —N(R¹)— or ═N— and ring A is a 5-membered heteroarylene, provided that at least one of X¹, X², and X³ is not ═C(H)—, or ═C(R¹)—. In some embodiments, X² is ═N— or —N(R¹)—, X¹ and X³ are each independently —O—, —S—, ═C(H)—, ═C(R¹)—, —N(H)—, or —N(R¹)—, and ring A is a 5-membered heteroarylene. In some embodiments, X² is ═N— or —N(R¹)—, X¹ and X³ are each independently ═C(H)—, ═C(R¹)—, and ring A is a 5-membered heteroarylene. In some embodiments, X¹, X², and X³ are each independently —N(R¹)—, ═N—, ═C(R¹)—, and ring A is a 5-membered heteroarylene, provided that at least one of X¹, X², and X³ is not ═C(R¹)—.

In some embodiments, Y¹, Y², and Y³ are each independently —O—, —S—, ═C(H)—, ═C(R²)—, —N(H)—, —N(R²)— or ═N— and ring B is a 5-membered heteroarylene, provided that at least one of Y¹, Y², and Y³ is not ═C(H)—, or ═C(R²)—. In some embodiments, Y¹, Y², and Y³ are each independently ═C(H)—, ═C(R²)—, —N(R²)— or ═N— and ring B is a 5-membered heteroarylene, provided that at least one of Y¹, Y², and Y³ is not ═C(H)—, or ═C(R²)—. In some embodiments, Y² is ═N—, Y¹ and Y³ are each independently —O—, —S—, ═C(H)—, ═C(R²)—, —N(H)—, or —N(R²)—, and ring B is a 5-membered heteroarylene. In some embodiments, ring A and ring B are each independently a 5-membered heteroarylene.

In some embodiments, ring A is selected from the group consisting of

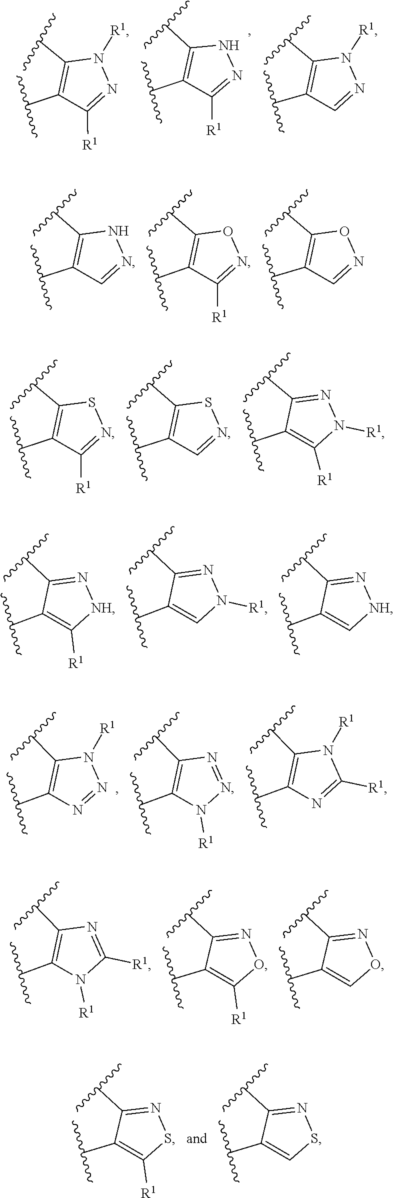

wherein each [symbol] represents a point of covalent attachment. In some embodiments, ring A is a pyrazolylene. In some embodiments, ring A is selected from the group consisting of

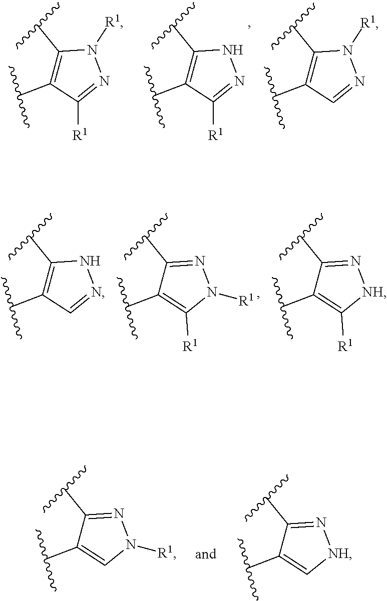

wherein each “ [symbol] ” represents a point of covalent attachment. In some embodiments, ring A is selected from the group consisting of

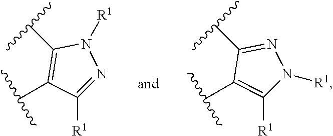

wherein each “ [symbol] ” represents a point of covalent attachment. In some embodiments, ring A is

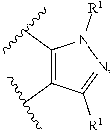

wherein each “ [symbol] ” represents a point of covalent attachment. In some embodiments, ring A is

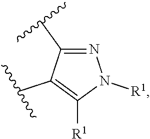

wherein each “ [symbol] ” represents a point of covalent attachment. In some embodiments, ring A is selected from the group consisting of

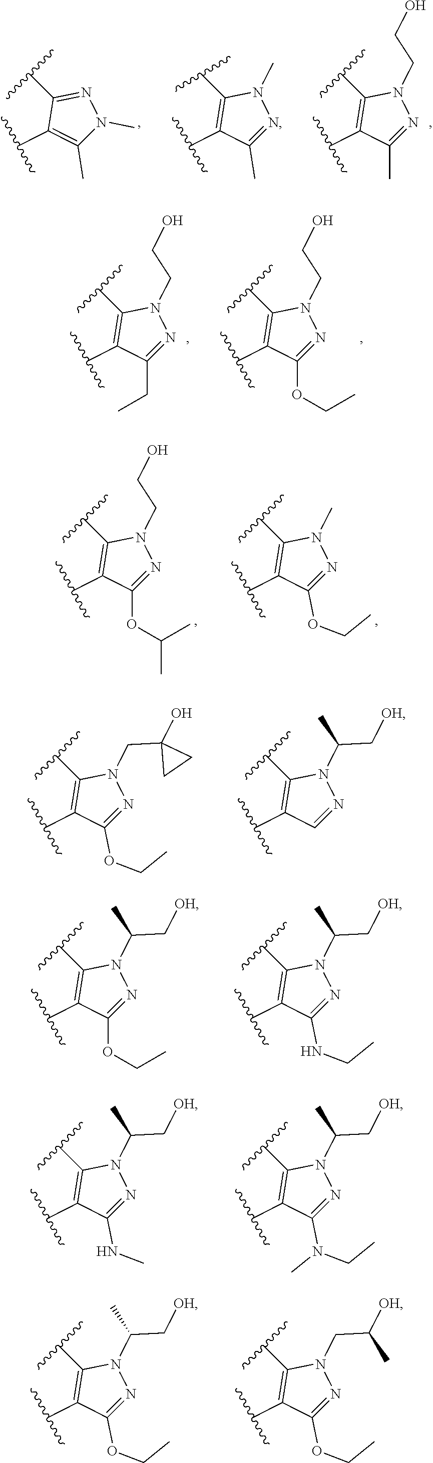

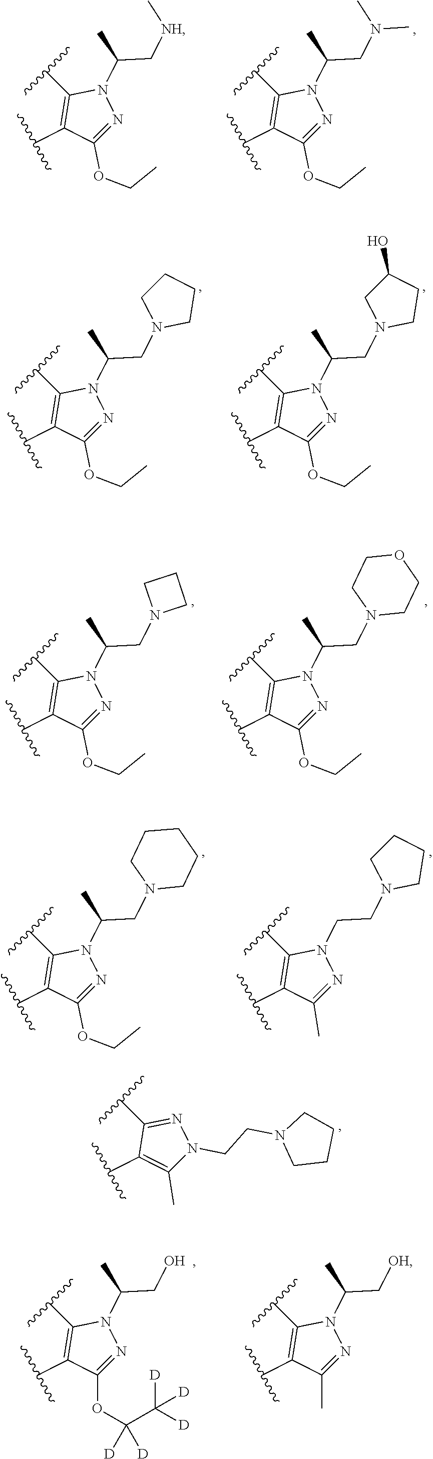

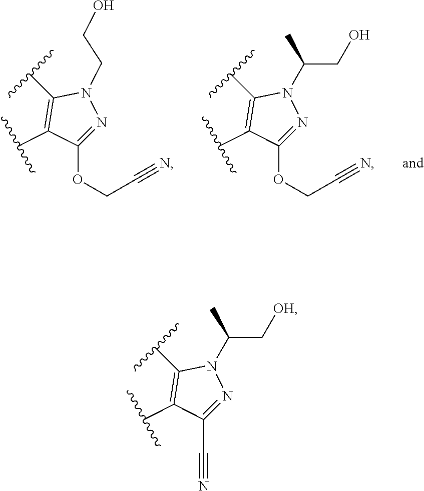 [SMILES: *c1c(OCC#N)nn(CCO)c1* ; *c1c(OCC#N)nn([C@@H](C)CO)c1* ; "and" ; *c1c(C#N)nn([C@@H](C)CO)c1*]

wherein each “ [symbol] ” represents a point of covalent attachment. In some embodiments, ring A is selected from the group consisting of

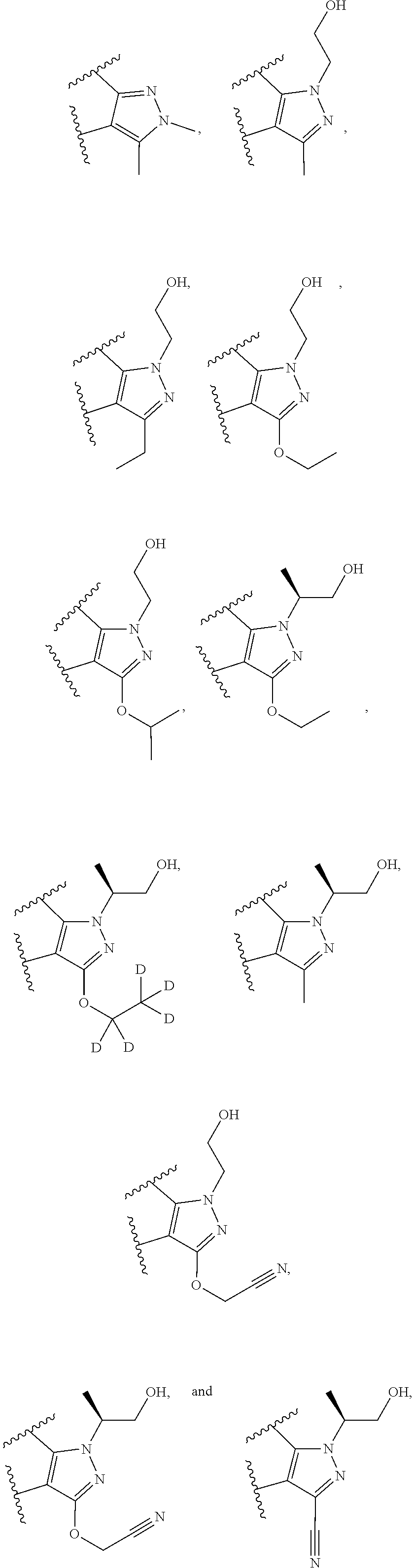

wherein each “ [symbol] ” represents a point of covalent attachment. In some embodiments, ring A is selected from the group consisting of

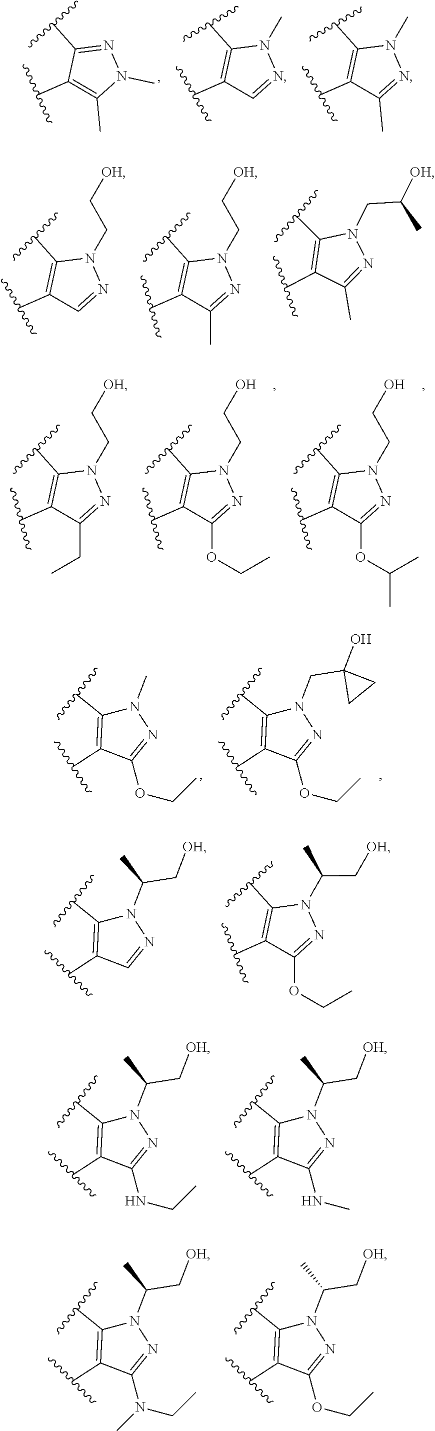

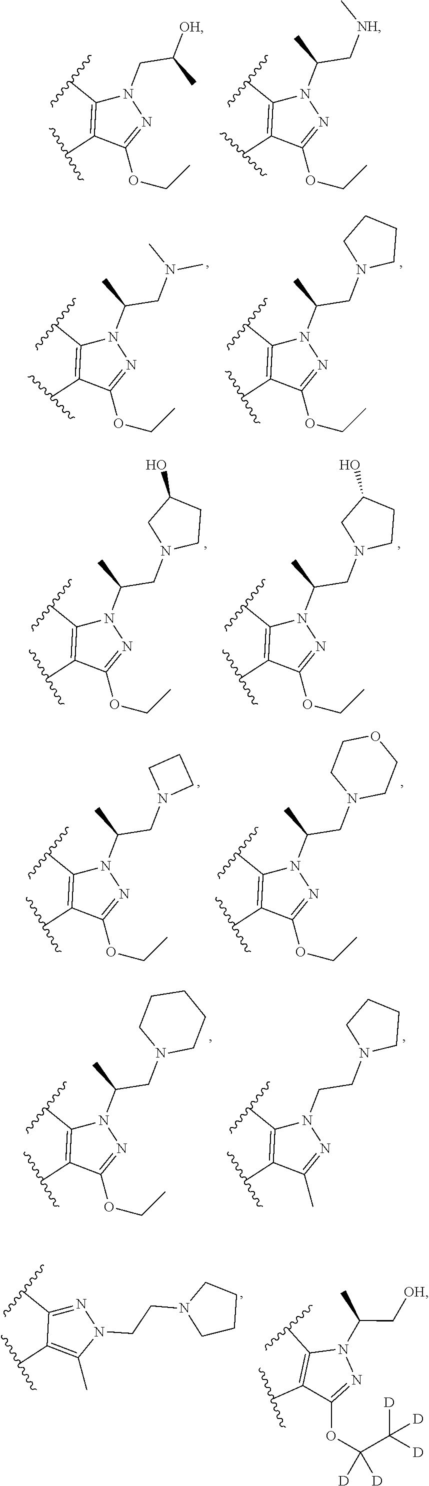

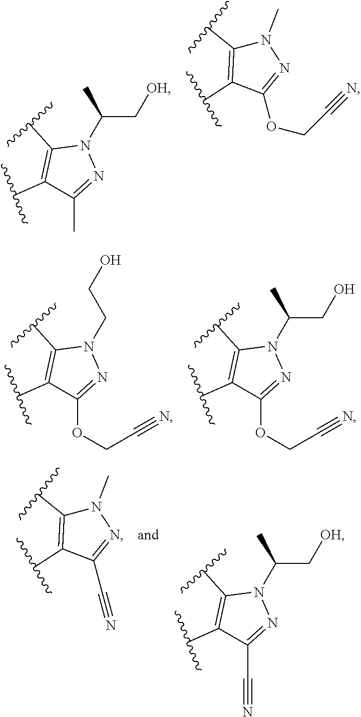

wherein each “ [symbol] ” represent a point of covalent attachment.

In some embodiments, ring B is selected from the group consisting of

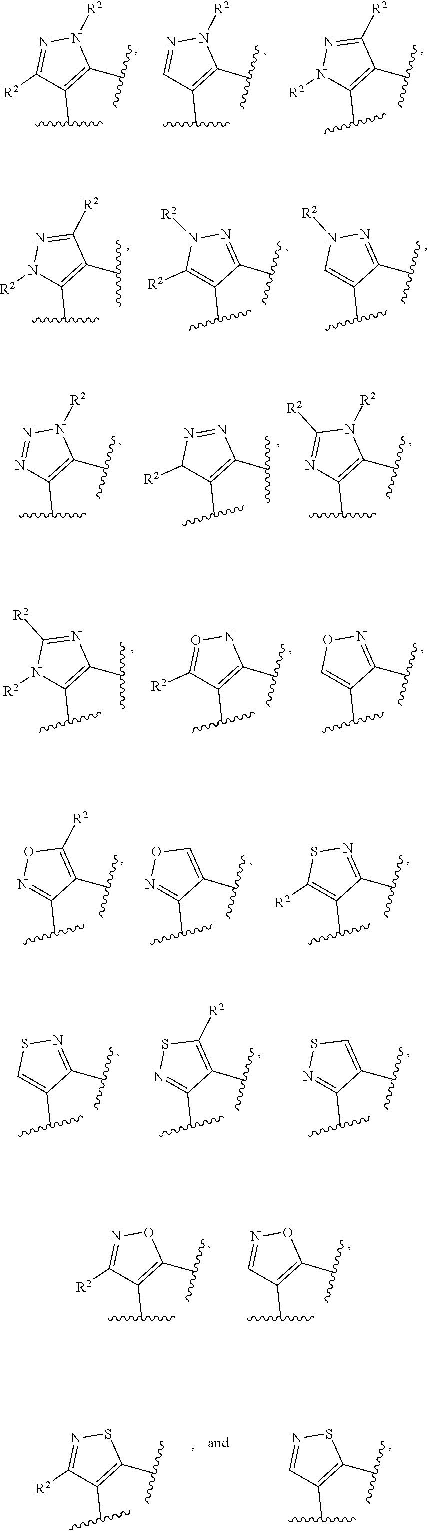

wherein each “ [symbol] ” represents a point of covalent attachment. In some embodiments, ring B is a pyrazolylene. In some embodiments, ring B is selected from the group consisting of

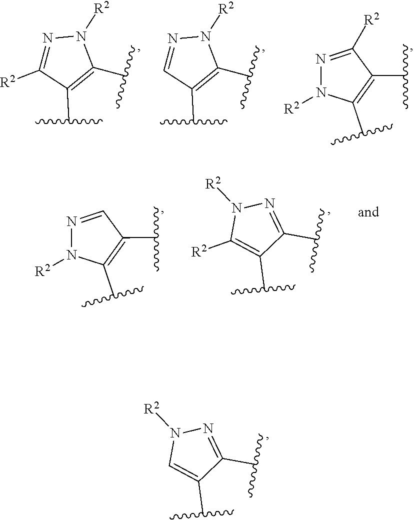

wherein each “ [symbol] ” represents a point of covalent attachment. In some embodiments, ring B is selected from the group consisting of

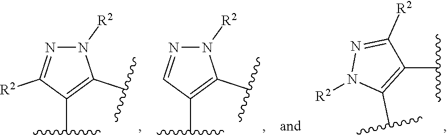

wherein each “ [symbol] ” represents a point of covalent attachment. In some embodiments, ring B is

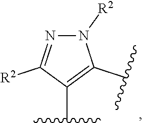

wherein each “ [symbol] ” represents a point of covalent attachment. In some embodiments, ring B is

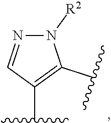

wherein each “ [symbol] ” represents a point of covalent attachment. In some embodiments, ring B is

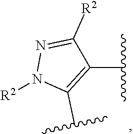

wherein each “ [symbol] ” represents a point of covalent attachment. In some embodiments, In some embodiments, ring B is selected from the group consisting of

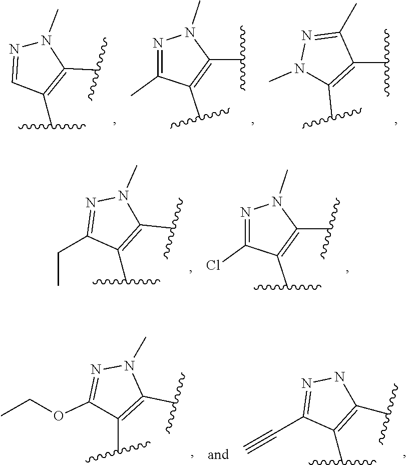

wherein each “ [symbol] ” represents a point of covalent attachment. In some embodiments, ring B is selected from the group consisting of

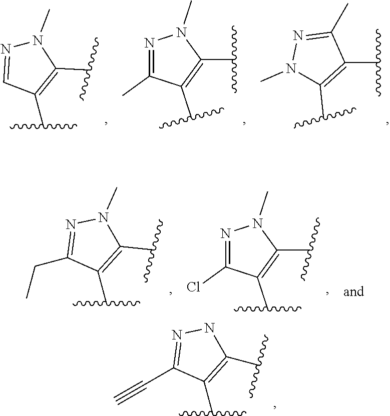

wherein each “ [symbol] ” represents a point of covalent attachment. In some embodiments,

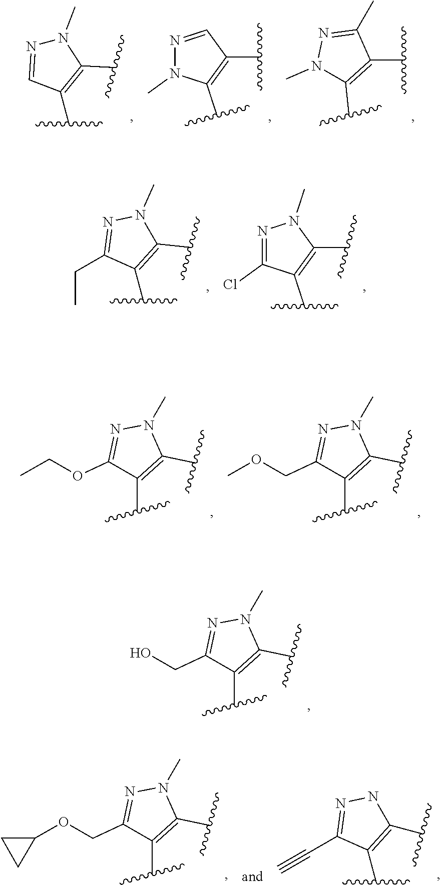

wherein each “ [symbol] ” represents a point of covalent attachment.

[symbol]

In some embodiments, ring A and ring B are each a pyrazolylene.

In some embodiments, m is 0, 1, 2, or 3. In some embodiments, m is 0, 1, or 2. In some embodiments, m is 0 or 1. In some embodiments, m is 1 or 2. In some embodiments, m is 2 or 3. In some embodiments, m is 0. In some embodiments, m is 1. In some embodiments, m is 2. In some embodiments, m is 3.

In some embodiments, n is 0, 1, 2, or 3. In some embodiments, n is 0, 1, or 2. In some embodiments, n is 0 or 1. In some embodiments, n is 1 or 2. In some embodiments, n is 2 or 3. In some embodiments, n is 0. In some embodiments, n is 1. In some embodiments, n is 2. In some embodiments, n is 3.

In some embodiments, each R¹ and R², when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{b}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{a}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{a}C(O)OR^{d}, —NR^{c}C(O)NR^{c}R^{d}, —NR^{a}C(═NR^{d})NR^{c}R^{d}, —NR^{c}S(O)R^{d}, —NR¹S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, each R¹ and R², when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, each R¹ and R², when present, is independently halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NRC(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR¹S(O)₂NR^{c}R^{d}, —C(O)R, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR′, —CN, or —NO₂.

In some embodiments, each R¹ and R², when present, is independently halogen, C₁-C₆ alkyl, C₂-C₆ alkenyl, C₂-C₆ alkynyl, C₃-C₆ cycloalkyl, 3- to 7-membered heterocycloalkyl, —OR^{a}, —OC(O)R^{a}, —OC(O)NR^{a}R^{b}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by halogen, C₁-C₆ alkyl, C₁-C₆ haloalkyl, —OR^{a}, —CN, or —NO₂. In some embodiments, each R¹ and R², when present, is independently halogen, C₁-C₆ alkyl, C₂-C₆ alkynyl, —OR^{a}, or —CN, wherein each hydrogen atom in C₁-C₆ alkyl and C₂-C₆ alkynyl is independently optionally substituted by halogen, C₁-C₆ alkyl, C₁-C₆haloalkyl, —OR^{a}, or —CN. In some embodiments, each R¹ and R², when present, is independently halogen, C₁-C₆ alkyl, C₂-C₆ alkynyl, —OR^{a}, or —CN, wherein each hydrogen atom in C₁-C₆ alkyl or C₂-C₆ alkynyl is independently optionally substituted by —OR^{c} or —CN.

In some embodiments, each R¹, when present, is independently C₁-C₆ alkyl, —OR^{a}, or —CN, wherein each hydrogen atom in C₁-C₆ alkyl is independently optionally substituted by —OR^{c} or —CN. In some embodiments, each R¹, when present, is independently C₁-C₆ alkyl, —OR^{a}, or —CN, wherein each hydrogen atom in C₁-C₆ alkyl is independently optionally substituted by —OH or —CN.

In some embodiments, each R², when present, is independently halogen, C₁-C₆ alkyl, or C₂-C₆ alkynyl. In some embodiments, each R², when present, is independently halogen, methyl, ethyl, or ethyne.

In some embodiments, each R^{a} is C₁-C₆ alkyl, wherein each hydrogen atom in C₁-C₆ alkyl is independently optionally substituted by deuterium or —CN. In some embodiments, each R^{a} is methyl, ethyl, or propyl, wherein each hydrogen atom in C₁-C₆ alkyl is independently optionally substituted by deuterium or —CN.

In some embodiments, each R^{c} is H.

In some embodiments, each R³, R⁴, R⁵, and R⁶ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{c}, —C(O)NR^{e}R^{f}, —PR^{c}R^{f}, —P(O)R^{c}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or two of R³, R⁴, R⁵, and R⁶, taken together with the carbon or carbons to which they are attached, combine to form C₃-C₆ cycloalkyl or 3- to 7-membered heterocycloalkyl, wherein each hydrogen atom in C₃-C₆ cycloalkyl or 3- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}—S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, each R³, R⁴, R⁵, and R⁶ is independently H or C₁-C₆ alkyl. In some embodiments, each R³, R⁴, R⁵, and R⁶ is independently H, methyl, or ethyl. In some embodiments, each R³, R⁴, R⁵, and R⁶ is independently H or methyl.

In some embodiments, each R³ and R⁴ is independently H, methyl, or ethyl. In some embodiments, each R³ and R⁴ is independently H or methyl. In some embodiments, one instance of R³ is methyl, and any remaining R³ and R⁴ are H or deuterium. In some embodiments, one instance of R³ is C₁-C₆alkyl; R⁷ and one instance of R⁴, taken together with the atoms to which they are attached, combine to form a 4- to 7-membered heterocycloalkyl; and any remaining R³ and R⁴ are H or deuterium.

In some embodiments, R⁵ and R⁶ are each H.

In some embodiments, R⁷ is H, deuterium, C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, 5- to 10-membered heteroaryl, —C(O)R^{c}, or —C(O)NR^{c}R^{d}, wherein each hydrogen atom in C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, and 5- to 10-membered heteroaryl is independently optionally substituted by —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{c}, —CN, or —NO₂; or R⁷ and one of R³, R⁴, R⁵, or R⁶, taken together with the atoms to which each is attached, combine to form a 4- to 7-membered heterocycloalkyl, wherein each hydrogen atom in the 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{c}S(O)₂NR^{e}R^{f}, —C(O)R^{c}, —C(O)OR^{c}, —C(O)NR^{e}R^{f}, —PR^{e}R^{f}, —P(O)R^{c}R^{f}, —P(O)₂R^{c}R^{f}, —P(O)NR^{e}R^{f}, —P(O)₂NR^{e}R^{f}, —P(O)OR^{e}, —P(O)₂₀R^{e}, —CN, or —NO₂.

In some embodiments, R⁷ is H, deuterium, C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, and 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{e})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NRCS(O)₂NR^{c}R^{d}, —C(O)R^{c}, —C(O)OR^{e}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; or R⁷ and one of R³, R⁴, R⁵, or R⁶, taken together with the atoms to which each is attached, combine to form a 4- to 7-membered heterocycloalkyl, wherein each hydrogen atom in the 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂.

In some embodiments, R⁷ is H, C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4-to 7-membered heterocycloalkyl, C₆-C₁₀ aryl, and 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{e}, —S(O)₂R^{c}, —S(O)NR^{e}R^{d}, —S(O)₂NR^{c}R^{d}, —NR^{c}R^{d}, —NR^{c}C(O)R^{d}, —N(C(O)R^{e})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{e}, —C(O)NR^{e}R^{d}, —C(═N)NR^{c}R^{d}, —PR^{c}R^{d}, —P(O)R^{c}R^{d}, —P(O)₂R^{c}R^{d}, —P(O)NR^{e}R^{d}, —P(O)₂NR^{c}R^{d}, —P(O)OR^{c}, —P(O)₂OR^{c}, —CN, or —NO₂; or R⁷ and one of R³, R⁴, R⁵, or R⁶, taken together with the atoms to which each is attached, combine to form a 4- to 7-membered heterocycloalkyl, wherein each hydrogen atom in the 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{c}R^{f}, —P(O)₂R^{c}R^{f}, —P(O)NR^{e}R^{f}, —P(O)₂NR^{e}R^{f}, —P(O)OR^{c}, —P(O)₂OR^{c}, —CN, or —NO₂.

In some embodiments, R⁷ is H, C₁-C₆ alkyl, C₃-C₆ cycloalkyl, or —C(O)R^{e}; or R⁷ and one instance of R³ or R⁴, taken together with the atoms to which they are attached, combine to form 4- to 7-membered heterocycloalkyl. In some embodiments, R⁷ is H, C₁-C₆ alkyl, C₃-C₆ cycloalkyl. In some embodiments, R⁷ and one of R³, R⁴, R⁵, or R⁶, taken together with the atoms to which each is attached, combine to form a 4- to 7-membered heterocycloalkyl. In some embodiments, R⁷ is H, methyl, ethyl, propyl, or cyclopropyl. In some embodiments, R⁷ is H, C₁-C₆ alkyl, or C₃-C₆ cycloalkyl. In some embodiments, R⁷ and one instance of R³ or R⁴, taken together with the atoms to which they are attached, combine to form 4- to 7-membered heterocycloalkyl. In some embodiments, R⁷ and one instance of R³ or R⁴, taken together with the atoms to which they are attached, combine to form 5-membered heterocycloalkyl.

In some embodiments, R⁷ is H, C₁-C₆ alkyl, C₃-C₆ cycloalkyl, or —C(O)R^{e}; or R⁷ and one instance of R³ or R⁴, taken together with the atoms to which they are attached, combine to form 4- to 7-membered heterocycloalkyl; wherein each hydrogen atom in C₁-C₆ alkyl, C₃-C₆ cycloalkyl, and 4- to 7-membered heterocycloalkyl is independently optionally substituted by —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, p is 2 or 3 and the portion

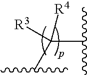

is an ethylene or propylene, wherein each R³ and R⁴ is independently H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or at least one instance of R³ and R⁴, taken together with the carbon or carbons to which they are attached, combine to form C₃-C₆ cycloalkyl or 4- to 7-membered heterocycloalkyl, wherein each hydrogen atom in C₃-C₆ cycloalkyl and 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R⁷ and one instance of R³ or R⁴, taken together with the atoms to which they are attached, combine to form 4- to 7-membered heterocycloalkyl, wherein each hydrogen atom in the 4- to 7-membered heterocycloalkyl formed is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R, —P(O)₂NR^{e}R^{f}, —P(O)OR^{e}, —P(O)₂OR^{e}, —CN, or —NO₂; and each “ [symbol] ” represents a point of covalent attachment.

In some embodiments, the portion

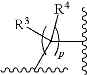

is an ethylene, wherein each R³ and R⁴ is independently H or C₁-C₆ alkyl; and/or R⁷ and one instance of R³ or R⁴, taken together with the atoms to which they are attached, combine to form 4- to 7-membered heterocycloalkyl.

In some embodiments, the portion

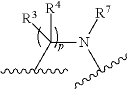

is of the formula

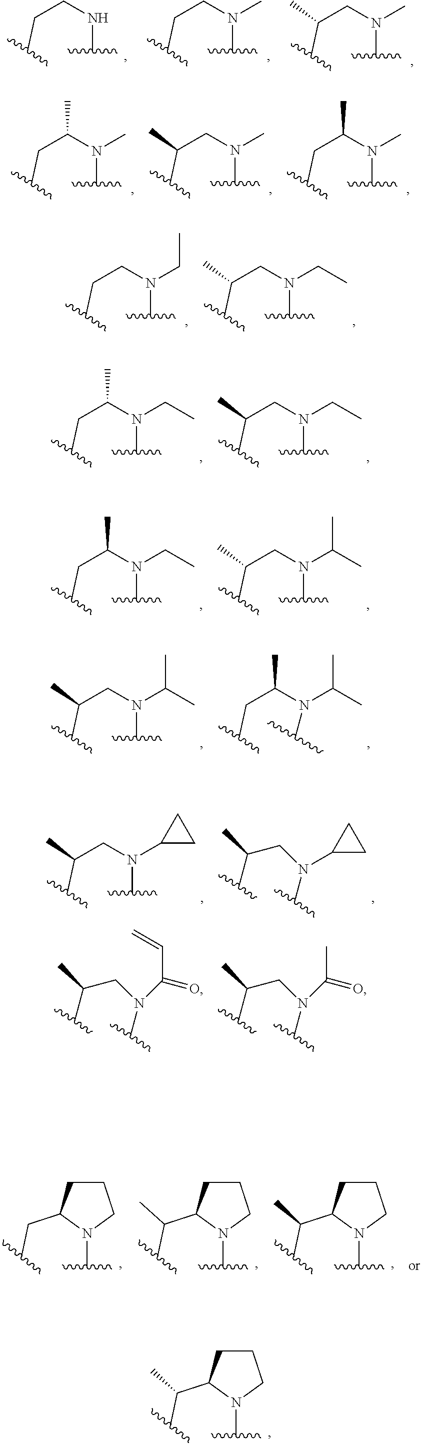

wherein each “ [symbol] ” represents a point of covalent attachment, and each hydrogen is independently optionally substituted with deuterium.

In some embodiments, the portion

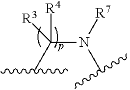

is of the formula

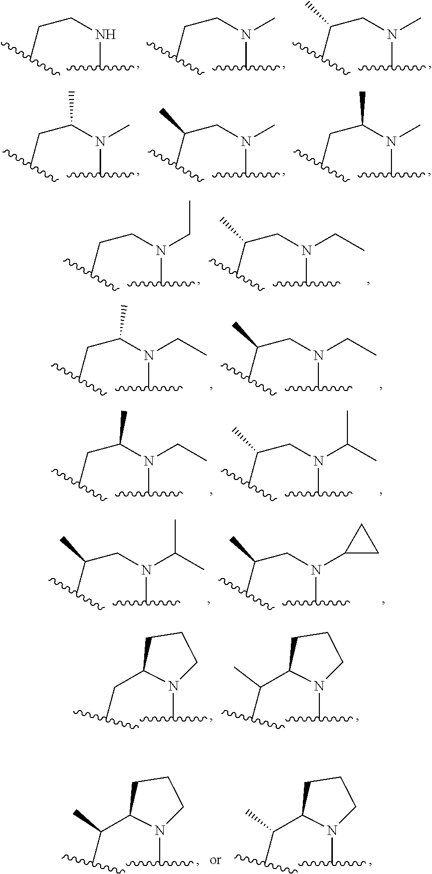

wherein each “ [symbol] ” represents a point of covalent attachment, and each hydrogen is independently optionally substituted with deuterium. In some embodiments, the portion

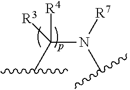

is of the formula

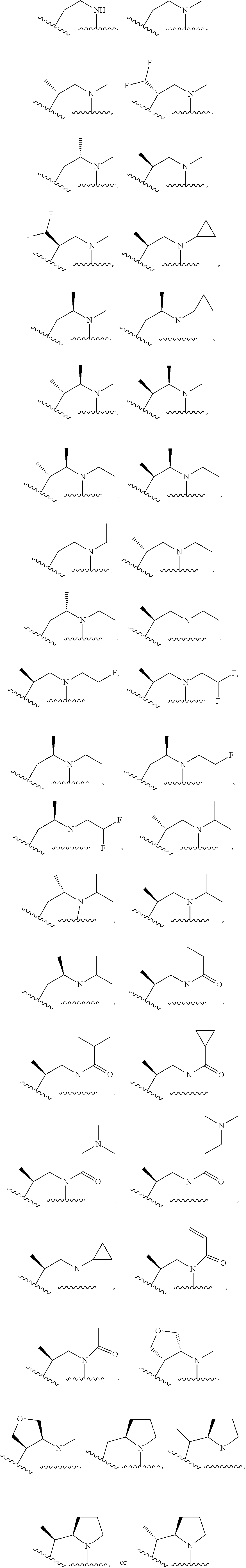

wherein each “ [symbol] ” represents a point of covalent attachment, and each hydrogen is independently optionally substituted with deuterium.

In some embodiments, p is 1, 2, 3, or 4. In some embodiments, p is 1, 2, or 3. In some embodiments, p is 2, 3, or 4. In some embodiments, p is 1 or 2. In some embodiments, p is 2 or 3. In some embodiments, p is 3 or 4. In some embodiments, p is 1. In some embodiments, p is 2. In some embodiments, p is 3. In some embodiments, p is 4.

In some embodiments, the portion

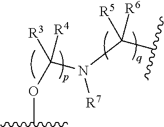

is of the formula

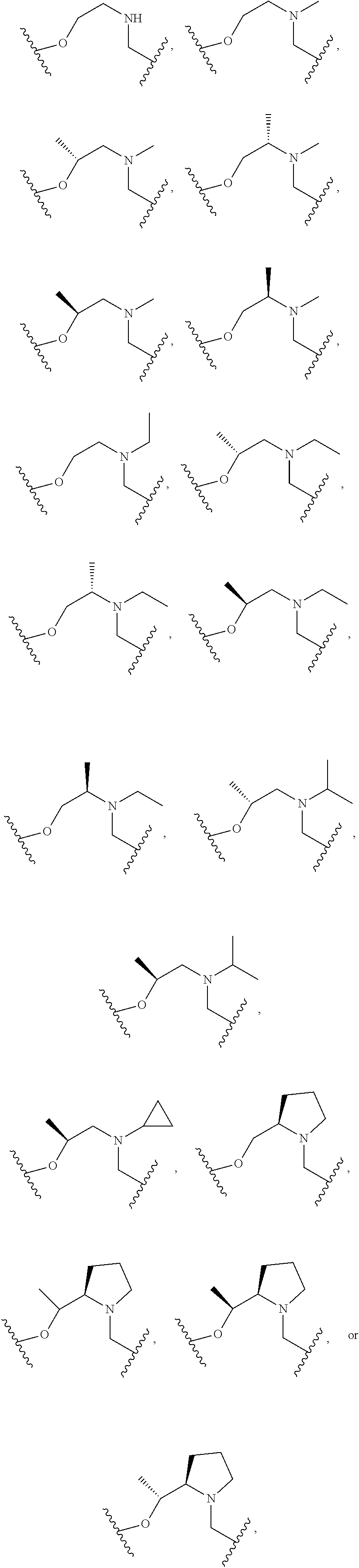

wherein each “ [symbol] ” represents a point of covalent attachment and each hydrogen is independently optionally substituted with deuterium.

In some embodiments, q is 1, 2, or 3. In some embodiments, q is 1 or 2. In some embodiments, q is 2 or 3. In some embodiments, q is 1. In some embodiments, q is 2. In some embodiments, q is 3.

In some embodiments, R⁸ is H, —S(O)₂R^{c}, —S(O)₂NR^{c}R^{d}, —C(O)R^{c}, —C(O)OR^{c}, —C(O)NR^{c}R^{d}, —C(═NR^{d})NR^{c}R^{d}, —P(O)₂R^{c}R^{d}, —P(O)₂NR^{c}R^{d}, or —P(O)₂OR^{c}.

In some embodiments, R⁸ is H, deuterium, —S(O)₂R^{c}, —S(O)₂NR^{c}R^{d}, —C(O)R^{c}, —C(O)OR^{c}, —C(O)NR^{c}R^{d}, —C(═N)NR^{c}R^{d}, —P(O)₂R^{c}R^{d}, —P(O)₂NR^{c}R^{d}, —P(O)₂OR^{c}. In some embodiments, R⁸ is H or C₁-C₆ alkyl. In some embodiments, R⁸ is H, —S(O)₂R^{c}, —S(O)₂NR^{c}R^{d}, —C(O)R^{c}, —C(O)OR^{c}, —C(O)NR^{c}R^{d}, —C(═N)NR^{c}R^{d}, —P(O)₂R^{c}R^{d}, —P(O)₂NR^{c}R^{d}, —P(O)₂OR^{c}. In some embodiments, R⁸ is H or C₁-C₆ alkyl. In some embodiments, R⁸ is H.

In some embodiments, R⁹ is H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, R⁹ is H, deuterium, halogen, C₁-C₆ alkyl, or —OR^{a}, wherein each hydrogen atom in C₁-C₆ alkyl is independently optionally substituted with deuterium. In some embodiments, R⁹ is H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR—, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C—(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R⁹ is H, deuterium, halogen, C₁-C₆ alkyl, wherein each hydrogen atom in C₁-C₆ alkyl is independently optionally substituted with deuterium. In some embodiments, R⁹ is C₁-C₆ alkyl, wherein each hydrogen atom in C₁-C₆ alkyl, is independently optionally substituted by deuterium. In some embodiments, R⁹ is H, deuterium, halogen, —CH₃, or —CD₃. In some embodiments, R⁹ is H, deuterium, halogen, —OCH₃, —CH₃, or —CD₃.

In some embodiments, each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl; or R^{a} and R^{b} or R^{e} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂.

In certain embodiments, R⁹ is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In certain embodiments, R⁹ is halogen, C₁-C₆ alkyl, C₂-C₆ alkenyl, C₂-C₆ alkynyl, C₃-C₆cycloalkyl, 3- to 7-membered heterocycloalkyl, C₆-C₁₀ aryl, 5- to 10-membered heteroaryl, —OR—, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R, —OS(O)₂R^{c}, —OS(O)NR^{c}R^{d}, —OS(O)₂NR^{c}R¹, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In certain embodiments, R⁹ is halogen, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂.

In certain embodiments, R⁹ is C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In certain embodiments, R⁹ is C₁-C₆ alkyl, wherein each hydrogen atom in C₁-C₆ alkyl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In certain embodiments, each of R⁸ and R⁹ is H. In other embodiments, R⁸ is H and R⁹ is not H. For example, in some embodiments, R⁸ is H and R⁹ is as described herein and may be deuterium, optionally substituted C₁-C₆ alkyl (e.g., methyl or —CD₃) or optionally substituted alkoxy (i.e., —O—C₁-C₆ alkyl) (e.g., methoxy).

In some embodiments, R⁸ is H, R⁹ is as described herein and may be deuterium, optionally substituted C₁-C₆ alkyl (e.g., methyl or —CD₃) or optionally substituted alkoxy (e.g., —O—C₁-C₆ alkyl such as methoxy), m is 2, and one R¹ is optionally substituted C₁-C₆ alkyl (e.g., unsubstituted such as methyl, hydroxy substituted such as —CH(CH₃)(CH₂OH) or ethanol, or heterocyloalkyl substituted such as -ethylene-pyrrolidinyl), and the other R¹ is either optionally substituted C₁-C₆ alkyl (e.g., ethyl, optionally substituted —N(H or C₁-C₆ alkyl)-C₁-C₆ alkyl) or optionally substituted —O—C₁-C₆ alkyl (e.g., ethoxy or —O—CH₂CN).

In some embodiments, R⁸ is H, R⁹ is optionally substituted C₁-C₆ alkyl (e.g., methyl or —CD₃) or optionally substituted alkoxy (e.g., —O—C₁-C₆ alkyl such as methoxy), m is 2, and one R¹ is optionally substituted C₁-C₆ alkyl (e.g., unsubstituted such as methyl, hydroxy substituted such as —CH(CH₃)(CH₂OH) or ethanol, or heterocyloalkyl substituted such as -ethylene-pyrrolidinyl), and the other R¹ is either optionally substituted C₁-C₆ alkyl (e.g., ethyl, optionally substituted —N(H or C₁-C₆ alkyl)-C₁-C₆ alkyl) or optionally substituted —O—C₁-C₆ alkyl (e.g., ethoxy or —O—CH₂CN), n is 2, and each R² is optionally substituted C₁-C₆ alkyl (e.g., unsubstituted such as methyl, hydroxy substituted such as —CH(CH₃)(CH₂OH), or ethanol).

In some embodiments, each of ring A and ring B are a pyrazole, and both of m and n are independently 1, 2, or 3. For example, m can be 1 or 2, and n can be 1 or 2. In some embodiments, each of ring A and ring B are a pyrazole, m is 1 or 2, and n is 2. In some embodiments, each of ring A and ring B are a pyrazole, m is 1, and n is 2. In some embodiments, each of ring A and ring B are a pyrazole, m is 2 and n is 2.

In some embodiments, p is 2 or 3, q is 1 or 2, and R⁷ is as described herein except is not H. For example, in some embodiments, p is 2 or 3, q is 1 or 2, and R⁷ is C₁-C₆ alkyl (e.g., methyl, ethyl, or isopropyl) or C₃-C₆ cycloalkyl (e.g., cyclopropyl).

In some embodiments, each of ring A and ring B are a pyrazole, each of m and n are independently 1, 2, or 3; R⁸ is H; and R⁹ is halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{c}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{c}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R¹, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; p is 2 or 3, q is 1 or 2, and R⁷ is C₁-C₆ alkyl (e.g., methyl, ethyl, or isopropyl) or C₃-C₆ cycloalkyl (e.g., cyclopropyl).

In some embodiments, each R^{a}, R^{b}, R^{c}, R^{d}, R^{e}, and R^{f} is independently selected from the group consisting of H, deuterium, and C₁-C₆ alkyl.

In some embodiments, the disclosure provides a compound of the formula (I)-(IV) selected from the group consisting of 2-[(10R,17E)-16-ethoxy-6,8,10-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11S,17E)-16-ethoxy-6,8,11-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-8,10-dimethyl-12-(propan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11S,17E)-8,11-dimethyl-12-(propan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-8,10,12-trimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-16-ethoxy-12-ethyl-8,10-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11S,17E)-16-ethoxy-12-ethyl-8,11-dimethyl-2,8,10,11,12,13-hexahydro-14H-5,3-(azenometheno)tripyrazolo[3,4-f:3′,4′-j:4″,3″-n][1,4]oxazacyclopentadecin-14-ylethan-1-ol;
2-[(10S,17E)-16-ethoxy-6,8,10-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-12-ethyl-8,10-dimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-12-ethyl-8,10-dimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-16-ethoxy-8,10-dimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-16-ethoxy-8,10-dimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-12-ethyl-6,8,10,16-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-12-ethyl-6,8,10,16-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11S,17E)-16-ethoxy-12-ethyl-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11R,17E)-16-ethoxy-12-ethyl-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethoxy-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
2-[(10R,17E)-6,8,10,16-tetramethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-6,8,10,16-tetramethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
{[(10S,17E)-14-(2-hydroxyethyl)-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(10S,17E)-12-ethyl-14-(2-hydroxyethyl)-6,8,10-trimethyl-2,10,11,12,13,14-hexahydro-8H-5,3-(azenometheno)tripyrazolol 3,4-f:3′,4′-j:4″,3″-n][1,4]oxazacyclopentadecin-16-yl]oxy}acetonitrile;
(2S)-2-[(10R,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(11S,17E)-16-ethoxy-12-ethyl-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,17E)-16-ethoxy-12-ethyl-6,8,10-trimethyl-2,8,10,11,12,13-hexahydro-14H-5,3-(azenometheno)tripyrazolo[3,4-f3′,4′-j:4″,3″-n][1,4]oxazacyclopentadecin-14-yl]propan-1-ol;
(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-01;
2-[(10S,17E)-12-cyclopropyl-16-ethoxy-8,10-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
{[(11S,17E)-14-(2-hydroxyethyl)-6,8,11,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
2-[(8aR,9S,19E)-1-ethoxy-9,11,13-trimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol;
2-[(8aR,9R,19E)-1-ethoxy-9,11,13-trimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol;
2-[(10S,17E)-16-ethoxy-12-ethyl-8,10-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10R,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-01;
{[(10R,17E)-12-ethyl-14-(2-hydroxyethyl)-6,8,10-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
2-[(8aR,19E)-1-ethoxy-11,13-dimethyl-7,8,8a,9,11,17-hexahydro-6H-14,16-(azenometheno)tripyrazolol3,4-f:3′,4′-j:4″,3″-n|pyrrolo|2,1-c|[1,4|oxazacyclopentadecin-3(4H)-yl]ethan-1-ol;
2-[(10S,17E)-6,8,10,12,16-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
{[(10R,17E)-14-(2-hydroxyethyl)-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(8aR,19E)-3-(2-hydroxyethyl)-11,13-dimethyl-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile;
2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-6,8,10,12,16-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(10S,17E)-14-[(2S)-1-hydroxypropan-2-yl]-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecine-16-carbonitrile;
{[(11R,17E)-14-(2-hydroxyethyl)-6,8,11,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(8aR,19E)-3-(2-hydroxyethyl)-9,11,13-trimethyl-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile;
{[(10S,17E)-14-[(2S)-1-hydroxypropan-2-yl]-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
2-[(10R,17E)-12,16-diethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(8aR,9S,19E)-1-ethoxy-9,11,13-trimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]propan-1-ol;
{[(10R,17E)-12-ethyl-14-[(2S)-1-hydroxypropan-2-yl]-6,8,10-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(17E)-16-ethoxy-6,8,12-trimethyl-2,8,10,11,12,13-hexahydro-14H-5,3-(azenometheno)tripyrazolol3,4-f:3′,4′-j:4″,3″-n][1,4]oxazacyclopentadecin-14-yl]propan-1-ol;
2-[(10S,17E)-16-ethyl-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethyl-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-01;
2-[(10S,17E)-12,16-diethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
{[(8aR,19E)-3-[(2S)-1-hydroxypropan-2-yl]-11,13-dimethyl-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile;
(2S)-2-[(8aR,19E)-1-ethoxy-11,13-dimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]propan-1-ol;
{[(17E)-12-ethyl-14-[(2S)-1-hydroxypropan-2-yl]-6,8-dimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(10S,17E)-12-ethyl-14-[(2S)-1-hydroxypropan-2-yl]-6,8,10-trimethyl-2,10,11,12,13,14-hexahydro-8H-5,3-(azenometheno)tripyrazolo[3,4-f3′,4′-j:4″,3″-n][1,4]oxazacyclopentadecin-16-yl]oxy}acetonitrile;
2-[(17E)-16-ethoxy-6,8,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(17E)-16-ethoxy-6,8,12-trimethyl-2,6,10,11,12,13-hexahydro-14H-5,3-(azenometheno)tripyrazolo[3,4-f:3′,4′-j:3″,4″-n][1,4]oxazacyclopentadecin-14-yl]ethan-1-ol;
{[(17E)-14-[(2S)-1-hydroxypropan-2-yl]-6,8,12-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(17E)-8,15,16-trimethyl-2,10,11,12,13,15-hexahydro-8H-5,3-(azenometheno)tripyrazolo[3,4-f:3′,4′-j:4″,3″-n][1,4]oxazacyclopentadecine;
(17E)-8,12,15,16-tetramethyl-2,10,11,12,13,15-hexahydro-8H-5,3-(azenometheno)tripyrazolo[3,4-f:3′,4′-j:4″,3″-n][1,4]oxazacyclopentadecine;
2-[(10S,17E)-16-ethoxy-6,12-diethyl-8,10-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethoxy-6-ethynyl-8,10,12-trimethyl-2,8,10,11,12,13-hexahydro-14H-5,3-(azenometheno)tripyrazolol3,4-f:3′,4′-j:4″,3″-n][1,4]oxazacyclopentadecin-14-yl]propan-1-ol;
2-[(10S,17E)-16-ethoxy-6-ethynyl-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethoxy-6-ethyl-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-1-[(10S,17E)-16-ethoxy-6,8,10,12-tetramethyl-2,8,10,11,12,13-hexahydro-14H-5,3-(azenometheno)tripyrazolo[3,4 f:3′,4′-j:4″,3″-n][1,4]oxazacyclopentadecin-14-yl]propan-2-ol;
(2S)-2-[(10S,17E)-16-[(²H5)ethyloxy]-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2R)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-01;
(2R)-2-[(10R,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-01;
(2S)-2-[(10S,17E)-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,17E)-16-(ethylamino)-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(11R,17E)-16-ethoxy-6,8,11,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
2-[(17E)-16-ethoxy-6,12-dimethyl-2,6,10,11,12,13-hexahydro-14H-5,3-(azenometheno)tripyrazolo[3,4-f:3′,4′-j:3″,4″-n][1,4]oxazacyclopentadecin-14-yl]ethan-1-ol;
(2S)-2-[(10S,17E)-16-[ethyl(methyl)amino]-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-1-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n∥1,4]oxazacyclopentadecin-14-yl]propan-2-ol;
(2S)-2-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(11R,17E)-12-cyclopropyl-16-ethoxy-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
1-[(10S,17E)-16-ethoxy-14-[(2S)-1-hydroxypropan-2-yl]-6,8,10-trimethyl-2,8,10,11,13,14-hexahydro-12H-5,3-(azenometheno)tripyrazolo[3,4-f:3′,4′-j:4″,3″-n][1,4]oxazacyclopentadecin-12-yl]ethan-1-one;
2-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11R,17E)-12-cyclopropyl-16-ethoxy-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
1-[(10S,17E)-16-ethoxy-14-[(2S)-1-hydroxypropan-2-yl]-6,8,10-trimethyl-2,8,10,11,13,14-hexahydro-12H-5,3-(azenometheno)tripyrazolo[3,4-f:3′,4′-j:4″,3″-n][1,4]oxazacyclopentadecin-12-yl]prop-2-en-1-one;
(10S,17E)-16-ethoxy-6,8,10,12-tetramethyl-14-[(2S)-1-(pyrrolidin-1-yl)propan-2-yl]-2,10,11,12,13,14-hexahydro-8H-5,3-(azenometheno)tripyrazolo[3,4-f:3′,4′-j:4″,3″-n][1,4]oxazacyclopentadecine;
(10S,17E)-14-[(2S)-1-(azetidin-1-yl)propan-2-yl]-16-ethoxy-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecine;
(10S,17E)-16-ethoxy-6,8,10,12-tetramethyl-14-[(2S)-1-(morpholin-4-yl)propan-2-yl]-2,10,11,12,13,14-hexahydro-8H-5,3-(azenometheno)tripyrazolo[3,4-f:3′,4′-j:4″,3″-n][1,4]oxazacyclopentadecine;
(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]-N-methylpropan-1-amine;
(2S)-2-[(10S,17E)-6,16-diethoxy-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(10S,17E)-16-ethoxy-6,8,10,12-tetramethyl-14-1(2S)-1-(piperidin-1-yl)propan-2-yl]-2,10,11,12,13,14-hexahydro-8H-5,3-(azenometheno)tripyrazolo[3,4-f:3′,4′-j:4″,3″-n][1,4]oxazacyclopentadecine;
(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]-N,N-dimethylpropan-1-amine;
(3S)-1-{(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propyl}pyrrolidin-3-ol;
(3R)-1-{(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propyl}pyrrolidin-3-ol;
1-{[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]methyl}cyclopropan-1-ol;
(2S)-2-[(10S,17E)-16-ethoxy-6-(methoxymethyl)-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol; and
(10S,17E)-6,8,10,12,15,16-hexamethyl-2,10,11,12,13,15-hexahydro-8H-5,3-(azenometheno)tripyrazolo[3,4-f:3′,4′-j:4″,3″-n][1,4]oxazacyclopentadecine;
   or a pharmaceutically acceptable salt, solvate, hydrate, or cocrystal thereof.

In some embodiments, the disclosure provides a compound of the formula (I)-(IV) selected from the group consisting of 2-[(10S,17E)-16-ethoxy-12-ethyl-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-16-ethoxy-8,10,12,20-tetramethyl-2,8,10,11,12,13-hexahydro-14H-5,3-(azenometheno)tripyrazolo[3,4-f:3′,4′-j:4″,3″-30n][1,4]oxazacyclopentadecin-14-yl]ethan-1-ol;
2-[(10R,17E)-16-ethoxy-12-ethyl-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,17E)-16-ethoxy-8,10,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-|(10S,17E)-8,10,12,20-tetramethyl-16-|(propan-2-yl)oxyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethoxy-8,10,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
2-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-t(10S,17E)-8,10,12,16,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
{[(10S,17E)-14-[(2S)-1-hydroxypropan-2-yl]-6,8,10,12,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
{[(10S,17E)-14-(2-hydroxyethyl)-6,8,10,12,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(10S,17E)-16-ethoxy-6,8,10,12-tetramethyl(20-²H)-10,11,12,13-tetrahydro-8H-5,3-(azenometheno)tripyrazolo[3,4-f:3′,4′-j:4″,3″-n][1,4]oxazacyclopentadecin-14(2H)-yl]propan-1-ol;
2-[(17E)-16-ethoxy-6,8,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-16-ethoxy-6,8,10,12-tetramethyl-20-(2H₃)methyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethoxy-6,8,10,12-tetramethyl-20-(²H3)methyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,17E)-16-[(²H₅)ethyloxy]-6,8,10,12,20-pentamethyl-2,8,10,11,12,13-hexahydro-14H-5,3-(azenometheno)tripyrazolo[3,4-f:3′,4′-j:4″,3″-n|[1,4]oxazacyclopentadecin-14-yl]propan-1-ol;
2-[(10S,17E)-16-ethoxy-6,8,10,12-tetramethyl(20-²H)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,17E)-16-ethoxy-12-ethyl-6,8,10-trimethyl(20-²H)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(10S,17E)-16-ethoxy-12-ethyl-6,8,10-trimethyl(20-²H)-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-1-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol;
(2S)-2-[(10S,17E)-16-ethoxy-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-01;
(2S)-2-[(10S,17E)-16-(ethylamino)-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2R)-2-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,17E)-16-ethoxy-20-methoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10R,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(10S,17E)-16-ethoxy-14-[(2S)-1-hydroxypropan-2-yl]-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-20-ol;
(2R)-2-[(10R,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-(10S,17E)-6,8,10,12,20-pentamethyl-16-(methylamino)-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,17E)-16-[ethyl(methyl)amino]-6,8,10,12,20-pentamethyl-2,8,10,11,12,13-hexahydro-14H-5,3-(azenometheno)tripyrazolo[3,4-f:3,4′-j:4″,3″-n][1,4]oxazacyclopentadecin-14-yl]propan-1-ol;
(10S,17E)-6,8,10,12,14,16,20-heptamethyl-2,10,11,12,13,14-hexahydro-8H-5,3-(azenometheno)tripyrazolo[3,4-f:3′,4′-j:4″,3″-n][1,4]oxazacyclopentadecine;
(10S,17E)-6,8,10,14,16,20-hexamethyl-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine;
(11R,17E)-6,8,11,14,16,20-hexamethyl-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine;
2-[(10S,17E)-6,8,10,16,20-pentamethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(10S,17E)-6,8,10,12,16,20-hexamethyl-14-[2-(pyrrolidin-1-yl)ethyl]-2,10,11,12,13,14-hexahydro-8H-5,3-(azenometheno)tripyrazolo[3,4-f:3′,4′-j:4″,3″-n][1,4]oxazacyclopentadecine;
(10S,17E)-6,8,10,12,16,20-hexamethyl-15-[2-(pyrrolidin-1-yl)ethyl]-2,10,11,12,13,15-hexahydro-8H-5,3-(azenometheno)tripyrazolo[3,4-f:3′,4′-j:4″,3″-n][1,4]oxazacyclopentadecine;
(2S)-2-[(10S,17E)-12-cyclopropyl-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(11R,17E)-12-cyclopropyl-6,8,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
1-[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]ethan-1-one;
(2S)-2-[(10R,11R,17E)-16-ethoxy-6,8,10,11,12,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-1 (10S,11R,17E)-16-ethoxy-6,8,10,11,12,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
2-[(10S,17E)-12-cyclopropyl-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(11R,17E)-12-cyclopropyl-6,8,11,20-tetramethyl-2,8,10,11,12,13-hexahydro-14H-5,3-(azenometheno)tripyrazolo[f:3′,4′-j:4″,3″-n][1,4]oxazacyclopentadecin-14-yl]ethan-1-ol;
1-[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]propan-1-one;
cyclopropyl[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]methanone;
1-[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]-2-methylpropan-1-one;
1-[(10S,17E)-6-[(cyclopropyloxy)methyl]-8,10,14,20-tetramethyl-2,8,10,11,13,14-hexahydro-12H-5,3-(azenometheno)tripyrazolo[3,4-f:3′,4′-j:4″,3″-n][1,4]oxazacyclopentadecin-12-yl]-2-(dimethylamino)ethan-1-one;
(2S)-2-[(10S,17E)-12-cyclopropyl-6-(methoxymethyl)-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(11R,17E)-12-cyclopropyl-6-(methoxymethyl)-8,11,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
2-(dimethylamino)-1-[(10S,17E)-6-(methoxymethyl)-8,10,14,20-tetramethyl-2,8,10,11,13,14-hexahydro-12H-5,3-(azenometheno)tripyrazolo[3,4-f:3′,4′-j:4″,3″-n][1,4]oxazacyclopentadecin-12-yl]ethan-1-one;
(17E)-6,8,12,15,16,20-hexamethyl-2,10,11,12,13,15-hexahydro-8H-5,3-(azenometheno)tripyrazolo[3,4-f:3′,4′-j:4″,3″-n][1,4]oxazacyclopentadecine;
3-(dimethylamino)-1-[(10S,17E)-6-(methoxymethyl)-8,10,14,20-tetramethyl-2,8,10,11,13,14-hexahydro-12H-5,3-(azenometheno)tripyrazolol3,4-f:3′,4′-j:4″,3″-n][1,4]oxazacyclopentadecin-12-yl]propan-1-one;
(17E)-6,8,12,14,16,20-hexamethyl-2,10,11,12,13,14-hexahydro-8H-5,3-(azenometheno)tripyrazolo[3,4-f:3′,4′-j:4″,3″-n][1,4]oxazacyclopentadecine;
(2S)-2-[(10S,17E)-16-ethoxy-12-ethyl-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecine;
(2S)-2-[(10S,17E)-16-ethoxy-12-ethyl-6-(methoxymethyl)-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
{[(10S,17E)-12-ethyl-14-[(2S)-1-hydroxypropan-2-yl]-6,8,10,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(10S,17E)-12-cyclopropyl-16-ethoxy-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecine-16-carbonitrile;
{[(10S,17E)-12-(2,2-difluoroethyl)-14-[(2S)-1-hydroxypropan-2-yl]-6,8,10,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(10S,17E)-12-cyclopropyl-14-[(2S)-1-hydroxypropan-2-yl]-6,8,10,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(11R,17E)-12-cyclopropyl-14-[(2S)-1-hydroxypropan-2-yl]-6,8,11,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(10S,17E)-12-cyclopropyl-16-ethoxy-6-(methoxymethyl)-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(11R,17E)-12-cyclopropyl-16-ethoxy-6-(methoxymethyl)-8,11,20-trimethyl-2,8,10,11,12,13-hexahydro-14H-5,3-(azenometheno)tripyrazolol3,4-f:3′,4′-j:4″,3″-n][1,4]oxazacyclopentadecin-14-yl]propan-1-ol;
[(11R,17E)-12-(2,2-difluoroethyl)-14-[(2S)-1-hydroxypropan-2-yl]-6,8,11,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(10S,17E)-16-ethoxy-6-(methoxymethyl)-8,10,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
{[(10S,17E)-12-(2-fluoroethyl)-14-[(2S)-1-hydroxypropan-2-yl]-6,8,10,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(11R,17E)-12-(2-fluoroethyl)-14-[(2S)-1-hydroxypropan-2-yl]-6,8,11,20-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(10S,17E)-6,8,10,12,14,20-hexamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
(2S)-2-[(10S,17E)-16-ethoxy-6-(hydroxymethyl)-8,10,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-1-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol;
(2S)-2-[(10R,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
(2S)-2-[(10S,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol;
{[(10R,17E)-10-(difluoromethyl)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile;
{[(10S,17E)-10-(difluoromethyl)-6,8,12,14,20-pentamethyl-2,10,11,12,13,14-hexahydro-8H-5,3-(azenometheno)tripyrazolol 3,4-f:3′,4′-j:4″,3″-n][1,4]oxazacyclopentadecin-16-yl]oxy}acetonitrile;
(10S,17E)-6,8,10,12,15,16,20-heptamethyl-2,10,11,12,13,15-hexahydro-8H-5,3-(azenometheno)tripyrazolo[3,4-f:3′,4′-j:4″,3″-n][1,4]oxazacyclopentadecine;
2-[(10S,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10R,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(2S)-2-[(4aS,7aS,13E)-12-ethoxy-1,3,8,21-tetramethyl-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol;
(2S)-2-[(4aR,7aR,13E)-12-ethoxy-1,3,8,21-tetramethyl-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol;
2-[(10R,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
2-[(10S,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol;
(10S,17E)-6-(methoxymethyl)-8,10,12,14,16,20-hexamethyl-2,10,11,12,13,14-hexahydro-8H-5,3-(azenometheno)tripyrazolo[3,4-f:3′,4′-j:4″,3″-n][1,4]oxazacyclopentadecine;
2-[(8aR,9S,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol;
2-[(8aR,9R,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol;
(2S)-2-[(8aR,9S,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]propan-1-ol;
(2S)-2-1(8aR,9R,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]propan-1-ol; and
(2S)-1-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol;
   or a pharmaceutically acceptable salt, solvate, hydrate, or cocrystal thereof.

The following represent illustrative embodiments of compounds of Formula (I)-(IV):

| Ex. | Structure | Name |
|---|---|---|
| 1 | 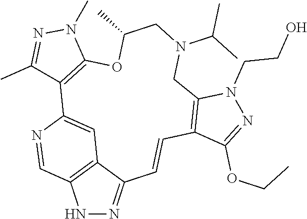 [SMILES: CCOc1nn(CCO)c2c1/C=C/c1n[nH]c3cnc(cc13)-c1c(C)nn(C)c1O[C@H](C)CN(C(C)C)C2] | 2-[(10R,17E)-16-ethoxy-6,8,10- trimethyl-12-(propan-2-yl)- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 2 | 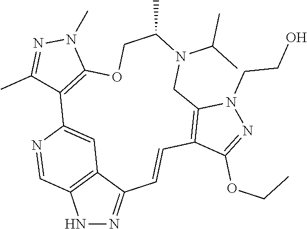 [SMILES: CCOc1nn(CCO)c2c1/C=C/c1n[nH]c3cnc(cc13)-c1c(C)nn(C)c1OC[C@H](C)N(C(C)C)C2] | 2-[(11S,17E)-16-ethoxy-6,8,11- trimethyl-12-(propan-2-yl)- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 3 | 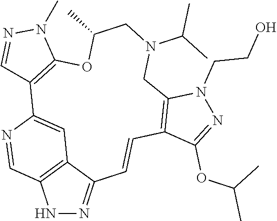 [SMILES: CC(C)Oc1nn(CCO)c2c1/C=C/c1n[nH]c3cnc(cc13)-c1cnn(C)c1O[C@H](C)CN(C(C)C)C2] | 2-[(10R,17E)-8,10-dimethyl-12- (propan-2-yl)-16-[(propan-2-yl)oxy]- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 4 | 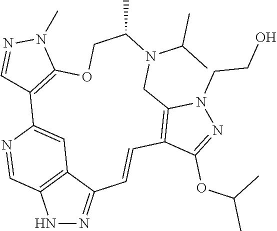 [SMILES: CC(C)Oc1nn(CCO)c2c1/C=C/c1n[nH]c3cnc(cc13)-c1cnn(C)c1OC[C@H](C)N(C(C)C)C2] | 2-[(11S,17E)-8,11-dimethyl-12- (propan-2-yl)-16-[(propan-2-yl)oxy]- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 5 | 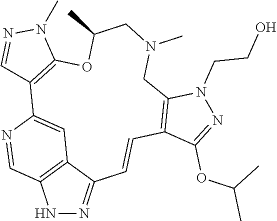 [SMILES: CC(C)Oc1nn(CCO)c2c1/C=C/c1n[nH]c3cnc(cc13)-c1cnn(C)c1O[C@@H](C)CN(C)C2] | 2-[(10S,17E)-8, 10,12-trimethyl-16- [(propan-2-yl)oxy]-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 6 | 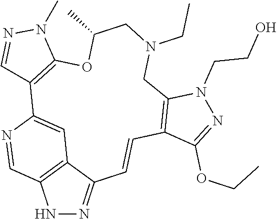 [SMILES: CCOc1nn(CCO)c2c1/C=C/c1n[nH]c3cnc(cc13)-c1cnn(C)c1O[C@H](C)CN(CC)C2] | 2-[(10R,17E)-16-ethoxy-12-ethyl-8,10- dimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]ethan-1-ol |
| 7 | 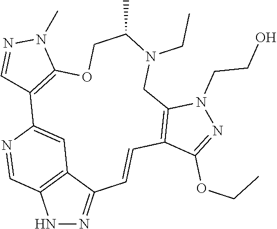 [SMILES: CCOc1nn(CCO)c2c1/C=C/c1n[nH]c3cnc(cc13)-c1cnn(C)c1OC[C@H](C)N(CC)C2] | 2-[(11S,17E)-16-ethoxy-12-ethyl-8,11- dimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]ethan-1-ol |
| 8 | 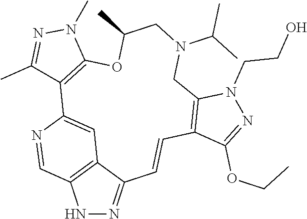 [SMILES: CCOc1nn(CCO)c2c1/C=C/c1n[nH]c3cnc(cc13)-c1c(C)nn(C)c1O[C@@H](C)CN(C(C)C)C2] | 2-[(10S,17E)-16-ethoxy-6,8,10- trimethyl-12-(propan-2-yl)- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 9 | 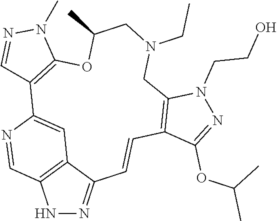 [SMILES: CCN1Cc2c(c(OC(C)C)nn2CCO)/C=C/c2n[nH]c3cnc(cc23)-c2cnn(C)c2O[C@@H](C)C1] | 2-[(10S,17E)-12-ethyl-8,10-dimethyl- 16-[(propan-2-yl)oxy]-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 10 | 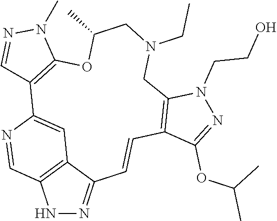 [SMILES: CCN1Cc2c(c(OC(C)C)nn2CCO)/C=C/c2n[nH]c3cnc(cc23)-c2cnn(C)c2O[C@H](C)C1] | 2-[(10R,17E)-12-ethyl-8,10-dimethyl- 16-[(propan-2-yl)oxy]-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 11 | 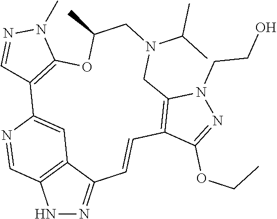 [SMILES: CCOc1nn(CCO)c2c1/C=C/c1n[nH]c3cnc(cc13)-c1cnn(C)c1O[C@@H](C)CN(C(C)C)C2] | 2-[(10S,17E)-16-ethoxy-8,10-dimethyl- 12-(propan-2-yl)-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 12 | 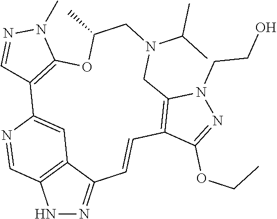 [SMILES: CCOc1nn(CCO)c2c1/C=C/c1n[nH]c3cnc(cc13)-c1cnn(C)c1O[C@H](C)CN(C(C)C)C2] | 2-[(10R,17E)-16-ethoxy-8,10- dimethyl-12-(propan-2-yl)- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 13 | 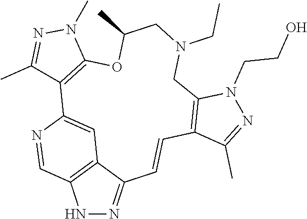 [SMILES: CCN1Cc2c(c(C)nn2CCO)/C=C/c2n[nH]c3cnc(cc23)-c2c(C)nn(C)c2O[C@@H](C)C1] | 2-[(10S,17E)-12-ethyl-6,8,10,16- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 14 | 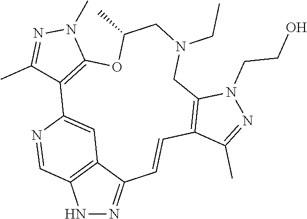 [SMILES: CCN1Cc2c(c(C)nn2CCO)/C=C/c2n[nH]c3cnc(cc23)-c2c(C)nn(C)c2O[C@H](C)C1] | 2-[(10R,17E)-12-ethyl-6,8,10,16- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 15 | 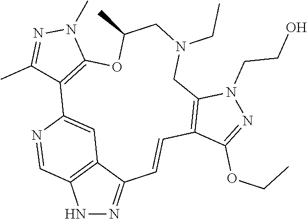 [SMILES: CCOc1nn(CCO)c2c1/C=C/c1n[nH]c3cnc(cc13)-c1c(C)nn(C)c1O[C@@H](C)CN(CC)C2] | 2-[(10S,17E)-16-ethoxy-12-ethyl- 6,8,10-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 16 | 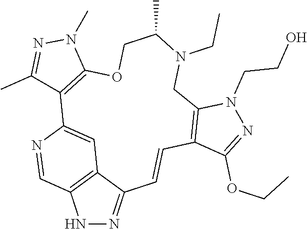 [SMILES: CCOc1nn(CCO)c2c1/C=C/c1n[nH]c3cnc(cc13)-c1c(C)nn(C)c1OC[C@H](C)N(CC)C2] | 2-[(11S,17E)-16-ethoxy-12-ethyl- 6,8,11-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 17 | 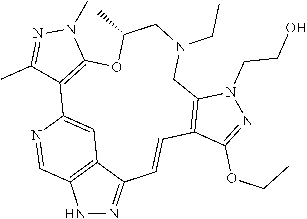 [SMILES: CCOc1nn(CCO)c2c1/C=C/c1n[nH]c3cnc(cc13)-c1c(C)nn(C)c1O[C@H](C)CN(CC)C2] | 2-[(10R,17E)-16-ethoxy-12-ethyl- 6,8,10-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 18 | 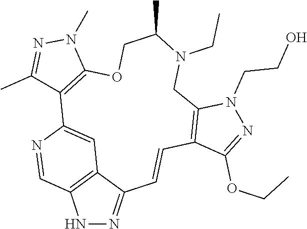 [SMILES: CCOc1nn(CCO)c2c1/C=C/c1n[nH]c3cnc(cc13)-c1c(C)nn(C)c1OC[C@@H](C)N(CC)C2] | 2-[(11R,17E)-16-ethoxy-12-ethyl- 6,8,11-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 19 | 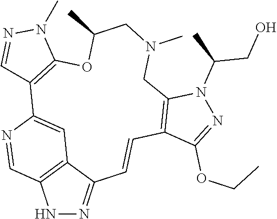 [SMILES: CCOc1nn([C@@H](C)CO)c2c1/C=C/c1n[nH]c3cnc(cc13)-c1cnn(C)c1O[C@@H](C)CN(C)C2] | (2S)-2-[(10S,17E)-16-ethoxy-8,10,12- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]propan-1-ol |
| 20 | 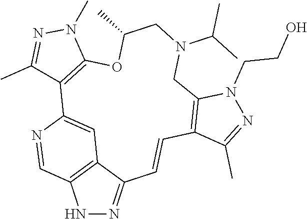 [SMILES: Cc1nn(CCO)c2c1/C=C/c1n[nH]c3cnc(cc13)-c1c(C)nn(C)c1O[C@H](C)CN(C(C)C)C2] | 2-[(10R,17E)-6,8,10,16-tetramethyl-12- (propan-2-yl)-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 21 | 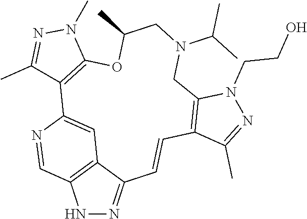 [SMILES: Cc1nn(CCO)c2c1/C=C/c1n[nH]c3cnc(cc13)-c1c(C)nn(C)c1O[C@@H](C)CN(C(C)C)C2] | 2-[(10S,17E)-6,8,10,16-tetramethyl-12- (propan-2-yl)-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 22 | 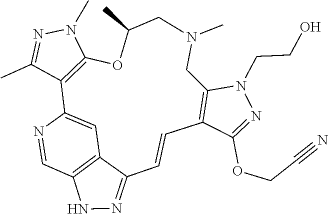 | {[(10S,17E)-14-(2-hydroxyethyl)- 6,8,10,12-tetramethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 23 | 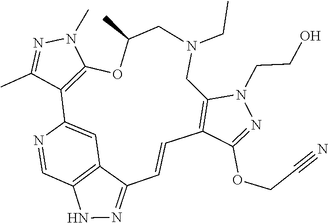 | {[(10S,17E)-12-ethyl-14-(2- hydroxyethyl)-6,8,10-trimethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 24 | 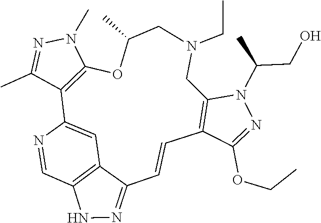 | (2S)-2-[(10R,17E)-16-ethoxy-12-ethyl- 6,8,10-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 25 | 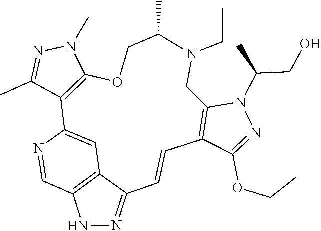 | (2S)-2-[(11S,17E)-16-ethoxy-12-ethyl- 6,8,11-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 26 | 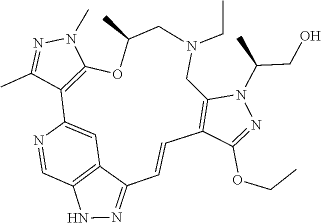 | (2S)-2-[(10S,17E)-16-ethoxy-12-ethyl- 6,8,10-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 27 | 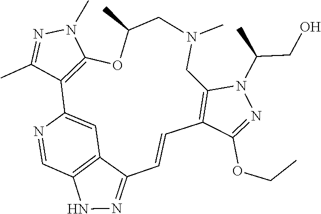 | (2S)-2-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 28 | 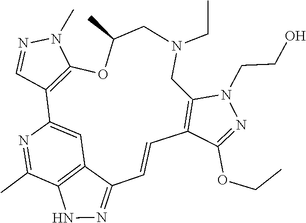 | 2-[(10S,17E)-16-ethoxy-12-ethyl- 8,10,20-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 29 | 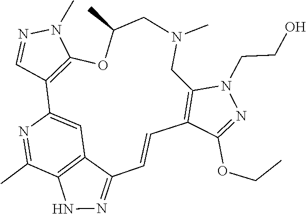 | 2-[(10S,17E)-16-ethoxy-8,10,12,20- tetramethyl-2,8,10,11,12,13- hexahydro-14H-5,3- (azenometheno)tripyrazolo[3,4-f:3′,4′- j:4″,3″-30n][1,4]oxazacyclopentadecin- 14-yl]ethan-1-ol |
| 30 | 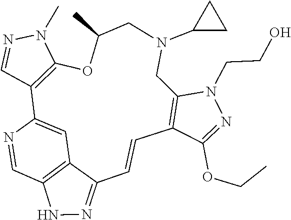 | 2-[(10S,17E)-12-cyclopropyl-16- ethoxy-8,10-dimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 31 | 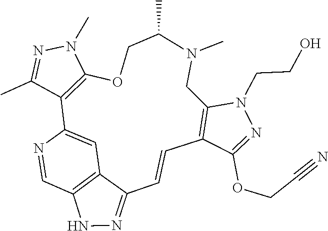 | {[(11S,17E)-14-(2-hydroxyethyl)- 6,8,11,12-tetramethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 32 | 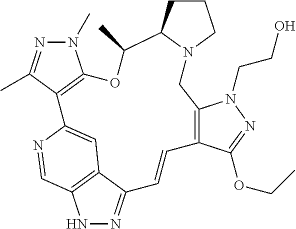 [SMILES: CCOc1nn(CCO)c2c1/C=C/c1n[nH]c3cnc(cc13)-c1c(C)nn(C)c1O[C@@H](C)[C@H]1CCCN1C2] | 2-[(8aR,9S,19E)-1-ethoxy-9,11,13- trimethyl-7,8,8a,9,11,17-hexahydro- 6H-14,16- (azenometheno)tripyrazolo[3,4-f:3′,4′- j:4″,3″-n]pyrrolo[2,1- c][1,4]oxazacyclopentadecin-3(4H)- yl]ethan-1-ol |
| 33 | 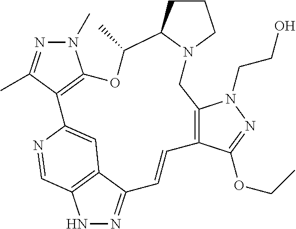 [SMILES: CCOc1nn(CCO)c2c1/C=C/c1n[nH]c3cnc(cc13)-c1c(C)nn(C)c1O[C@H](C)[C@H]1CCCN1C2] | 2-[(8aR,9R,19E)-1-ethoxy-9,11,13- trimethyl-7,8,8a,9,11,17-hexahydro- 6H-14,16- (azenometheno)tripyrazolo[3,4-f:3′,4′- j:4″,3″-n]pyrrolo[2,1- c][1,4]oxazacyclopentadecin-3(4H)- yl]ethan-1-ol |
| 34 | 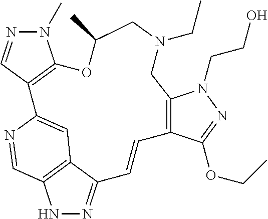 [SMILES: CCOc1nn(CCO)c2c1/C=C/c1n[nH]c3cnc(cc13)-c1cnn(C)c1O[C@@H](C)CN(CC)C2] | 2-[(10S,17E)-16-ethoxy-12-ethyl-8,10- dimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]ethan-1-ol |
| 35 | 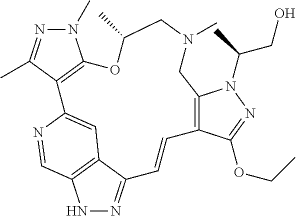 [SMILES: CCOc1nn([C@@H](C)CO)c2c1/C=C/c1n[nH]c3cnc(cc13)-c1c(C)nn(C)c1O[C@H](C)CN(C)C2] | (2S)-2-[(10R,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 36 | 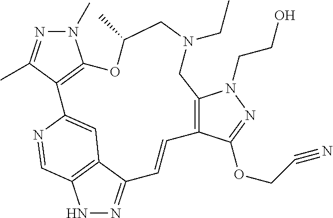 [SMILES: CCN1Cc2c(c(OCC#N)nn2CCO)/C=C/c2n[nH]c3cnc(cc23)-c2c(C)nn(C)c2O[C@H](C)C1] | {[(10R,17E)-12-ethyl-14-(2- hydroxyethyl)-6,8,10-trimethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 37 | 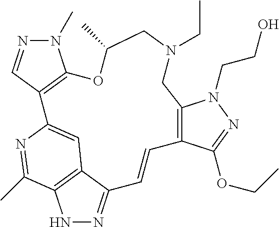 [SMILES: CCOc1nn(CCO)c2c1/C=C/c1n[nH]c3c(C)nc(cc13)-c1cnn(C)c1O[C@H](C)CN(CC)C2] | 2-[(10R,17E)-16-ethoxy-12-ethyl- 8,10,20-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 38 | 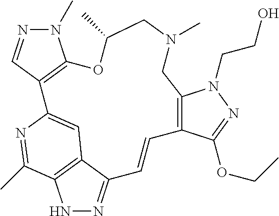 [SMILES: CCOc1nn(CCO)c2c1/C=C/c1n[nH]c3c(C)nc(cc13)-c1cnn(C)c1O[C@H](C)CN(C)C2] | 2-[(10R,17E)-16-ethoxy-8,10,12,20- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 39 | 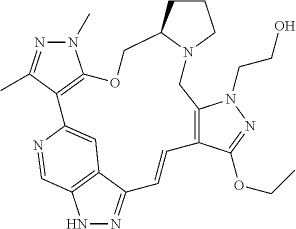 [SMILES: CCOc1nn(CCO)c2c1/C=C/c1n[nH]c3cnc(cc13)-c1c(C)nn(C)c1OC[C@H]1CCCN1C2] | 2-[(8aR,19E)-1-ethoxy-11,13-dimethyl- 7,8,8a,9,11,17-hexahydro-6H-14,16- (azenometheno)tripyrazolo[3,4-f:3′,4′- j:4″,3″-n]pyrrolo[2,1- c][1,4]oxazacyclopentadecin-3(4H)- yl]ethan-1-ol |
| 40 | 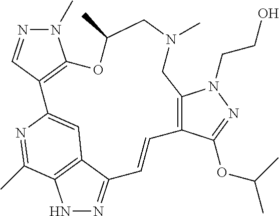 [SMILES: Cc1nc2cc3c(n[nH]c13)/C=C/c1c(OC(C)C)nn(CCO)c1CN(C)C[C@H](C)Oc1c-2cnn1C] | 2-[(10S,17E)-8,10,12,20-tetramethyl- 16-[(propan-2-yl)oxy]-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 41 | 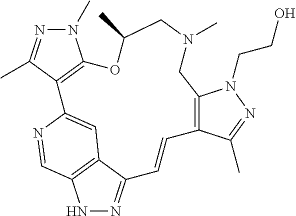 [SMILES: Cc1nn(CCO)c2c1/C=C/c1n[nH]c3cnc(cc13)-c1c(C)nn(C)c1O[C@@H](C)CN(C)C2] | 2-[(10S,17E)-6,8,10,12,16- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 42 | 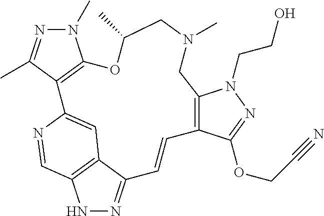 [SMILES: Cc1nn(C)c2c1-c1cc3c(n[nH]c3cn1)/C=C/c1c(OCC#N)nn(CCO)c1CN(C)C[C@@H](C)O2] | {[(10R,17E)-14-(2-hydroxyethyl)- 6,8,10,12-tetramethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 43 | 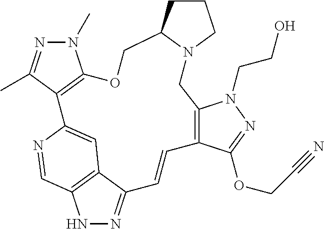 [SMILES: Cc1nn(C)c2c1-c1cc3c(n[nH]c3cn1)/C=C/c1c(OCC#N)nn(CCO)c1CN1CCC[C@@H]1CO2] | {[(8aR,19E)-3-(2-hydroxyethyl)-11,13- dimethyl-3,4,7,8,8a,9,11,17-octahydro- 6H-14,16- (azenometheno)tripyrazolo[3,4-f:3′,4′- j:4″,3″-n]pyrrolo[2,1- c][1,4]oxazacyclopentadecin-1- yl]oxy}acetonitrile |
| 44 | 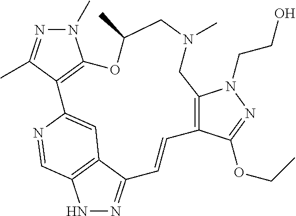 [SMILES: CCOc1nn(CCO)c2c1/C=C/c1n[nH]c3cnc(cc13)-c1c(C)nn(C)c1O[C@@H](C)CN(C)C2] | 2-[(10S,17E)-16-ethoxy-6,8,10,12- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 45 | 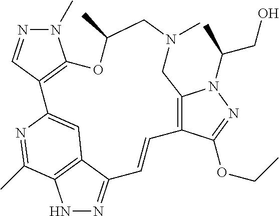 [SMILES: CCOc1nn([C@@H](C)CO)c2c1/C=C/c1n[nH]c3c(C)nc(cc13)-c1cnn(C)c1O[C@@H](C)CN(C)C2] | (2S)-2-[(10S,17E)-16-ethoxy- 8,10,12,20-tetramethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 46 | 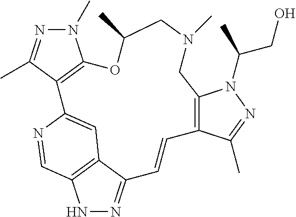 [SMILES: Cc1nn([C@@H](C)CO)c2c1/C=C/c1n[nH]c3cnc(cc13)-c1c(C)nn(C)c1O[C@@H](C)CN(C)C2] | (2S)-2-[(10S,17E)-6,8,10,12,16- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 47 | 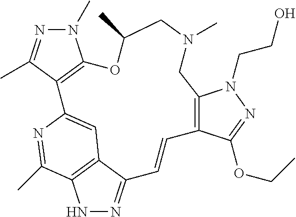 [SMILES: CCOc1nn(CCO)c2c1/C=C/c1n[nH]c3c(C)nc(cc13)-c1c(C)nn(C)c1O[C@@H](C)CN(C)C2] | 2-[(10S,17E)-16-ethoxy-6,8,10,12,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 48 | 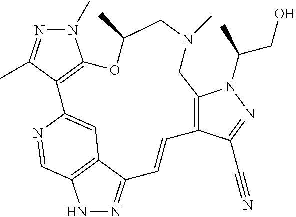 [SMILES: Cc1nn(C)c2c1-c1cc3c(n[nH]c3cn1)/C=C/c1c(C#N)nn([C@@H](C)CO)c1CN(C)C[C@H](C)O2] | (10S,17E)-14-[(2S)-1-hydroxypropan- 2-yl]-6,8,10,12-tetramethyl- 2,10,11,12,13,14-hexahydro-8H-5,3- (azenometheno)tripyrazolo[3,4-f:3′,4′- j:4″,3″-n][1,4]oxazacyclopentadecine- 16-carbonitrile |
| 49 | 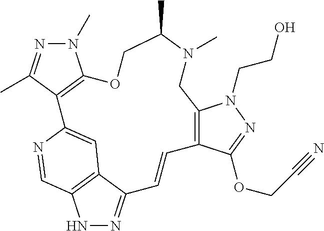 [SMILES: Cc1nn(C)c2c1-c1cc3c(n[nH]c3cn1)/C=C/c1c(OCC#N)nn(CCO)c1CN(C)[C@H](C)CO2] | {[(11R,17E)-14-(2-hydroxyethyl)- 6,8,11,12-tetramethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 50 | 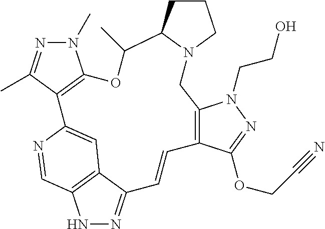 [SMILES: Cc1nn(C)c2c1-c1cc3c(n[nH]c3cn1)/C=C/c1c(OCC#N)nn(CCO)c1CN1CCC[C@@H]1C(C)O2] | {[(8aR,19E)-3-(2-hydroxyethyl)- 9,11,13-trimethyl-3,4,7,8,8a,9,11,17- octahydro-6H-14,16- (azenometheno)tripyrazolo[3,4-f:3′,4′- j:4″,3″-n]pyrrolo[2,1- c][1,4]oxazacyclopentadecin-1- yl]oxy}acetonitrile |
| 51 | 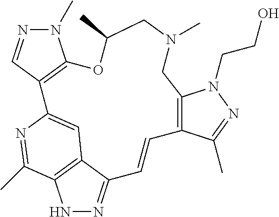 [SMILES: Cc1nn(CCO)c2c1/C=C/c1n[nH]c3c(C)nc(cc13)-c1cnn(C)c1O[C@@H](C)CN(C)C2] | 2-[(10S,17E)-8,10,12,16,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 52 | 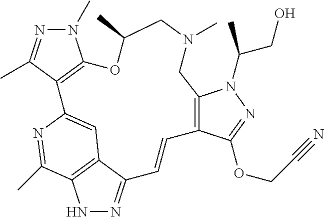 [SMILES: Cc1nn(C)c2c1-c1cc3c(n[nH]c3c(C)n1)/C=C/c1c(OCC#N)nn([C@@H](C)CO)c1CN(C)C[C@H](C)O2] | {[(10S,17E)-14-[(2S)-1- hydroxypropan-2-yl]-6,8,10,12,20- pentamethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 53 | 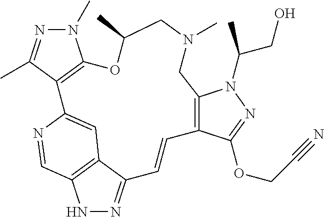 [SMILES: Cc1nn(C)c2c1-c1cc3c(n[nH]c3cn1)/C=C/c1c(OCC#N)nn([C@@H](C)CO)c1CN(C)C[C@H](C)O2] | {[(10S,17E)-14-[(2S)-1- hydroxypropan-2-yl]-6,8,10,12- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 54 | 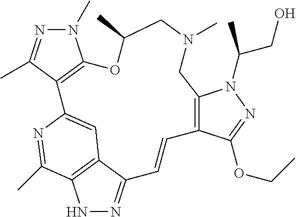 [SMILES: CCOc1nn([C@@H](C)CO)c2c1/C=C/c1n[nH]c3c(C)nc(cc13)-c1c(C)nn(C)c1O[C@@H](C)CN(C)C2] | (2S)-2-[(10S,17E)-16-ethoxy- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 55 | 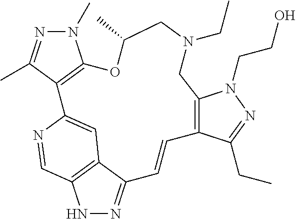 [SMILES: CCc1nn(CCO)c2c1/C=C/c1n[nH]c3cnc(cc13)-c1c(C)nn(C)c1O[C@H](C)CN(CC)C2] | 2-[(10R,17E)-12,16-diethyl-6,8,10- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]ethan-1-ol |
| 56 | 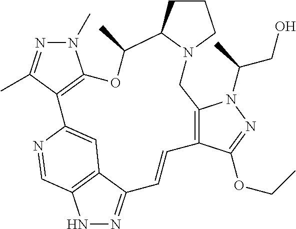 [SMILES: CCOc1nn([C@@H](C)CO)c2c1/C=C/c1n[nH]c3cnc(cc13)-c1c(C)nn(C)c1O[C@@H](C)[C@H]1CCCN1C2] | (2S)-2-[(8aR,9S,19E)-1-ethoxy- 9,11,13-trimethyl-7,8,8a,9,11,17- hexahydro-6H-14,16- (azenometheno)tripyrazolo[3,4-f:3′,4′- j:4″,3″-n]pyrrolo[2,1- c][1,4]oxazacyclopentadecin-3(4H)- yl]propan-1-ol |
| 57 | 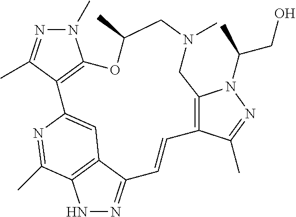 [SMILES: Cc1nn([C@@H](C)CO)c2c1/C=C/c1n[nH]c3c(C)nc(cc13)-c1c(C)nn(C)c1O[C@@H](C)CN(C)C2] | (2S)-2-[(10S,17E)-6,8,10,12,16,20- hexamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 58 | 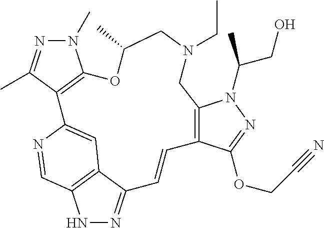 [SMILES: CCN1Cc2c(c(OCC#N)nn2[C@@H](C)CO)/C=C/c2n[nH]c3cnc(cc23)-c2c(C)nn(C)c2O[C@H](C)C1] | {[(10R,17E)-12-ethyl-14-[(2S)-1- hydroxypropan-2-yl]-6,8,10-trimethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 59 | 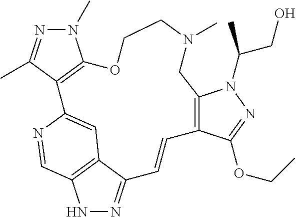 [SMILES: CCOc1nn([C@@H](C)CO)c2c1/C=C/c1n[nH]c3cnc(cc13)-c1c(C)nn(C)c1OCCN(C)C2] | (2S)-2-[(17E)-16-ethoxy-6,8,12- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]propan-1-ol |
| 60 | 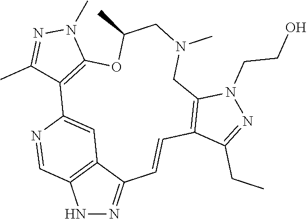 [SMILES: CCc1nn(CCO)c2c1/C=C/c1n[nH]c3cnc(cc13)-c1c(C)nn(C)c1O[C@@H](C)CN(C)C2] | 2-[(10S,17E)-16-ethyl-6,8,10,12- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 61 | 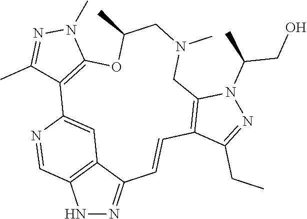 [SMILES: CCc1nn([C@@H](C)CO)c2c1/C=C/c1n[nH]c3cnc(cc13)-c1c(C)nn(C)c1O[C@@H](C)CN(C)C2] | (2S)-2-[(10S,17E)-16-ethyl-6,8,10,12- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 62 | 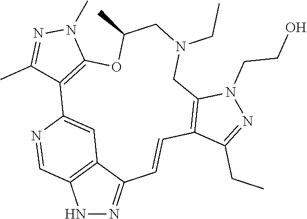 [SMILES: CCc1nn(CCO)c2c1/C=C/c1n[nH]c3cnc(cc13)-c1c(C)nn(C)c1O[C@@H](C)CN(CC)C2] | 2-[(10S,17E)-12,16-diethyl-6,8,10- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]ethan-1-ol |
| 63 | 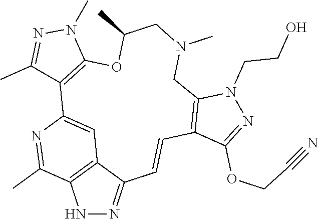 [SMILES: Cc1nn(C)c2c1-c1cc3c(n[nH]c3c(C)n1)/C=C/c1c(OCC#N)nn(CCO)c1CN(C)C[C@H](C)O2] | {[(10S,17E)-14-(2-hydroxyethyl)- 6,8,10,12,20-pentamethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 64 | 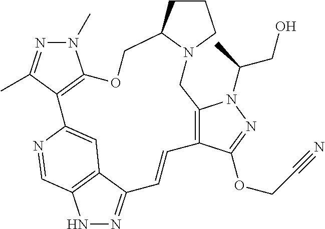 [SMILES: Cc1nn(C)c2c1-c1cc3c(n[nH]c3cn1)/C=C/c1c(OCC#N)nn([C@@H](C)CO)c1CN1CCC[C@@H]1CO2] | {[(8aR,19E)-3-[(2S)-1-hydroxypropan- 2-yl]-11,13-dimethyl- 3,4,7,8,8a,9,11,17-octahydro-6H- 14,16-(azenometheno)tripyrazolo[3,4- f:3′,4′-j:4″,3″-n]pyrrolo[2,1- c][1,4]oxazacyclopentadecin-1- yl]oxy}acetonitrile |
| 65 | 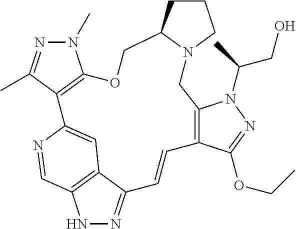 [SMILES: CCOc1nn([C@@H](C)CO)c2c1/C=C/c1n[nH]c3cnc(cc13)-c1c(C)nn(C)c1OC[C@H]1CCCN1C2] | (2S)-2-[(8aR,19E)-1-ethoxy-11,13- dimethyl-7,8,8a,9,11,17-hexahydro- 6H-14,16- (azenometheno)tripyrazolo[3,4-f:3′,4′- j:4″,3″-n]pyrrolo[2,1- c][1,4]oxazacyclopentadecin-3(4H)- yl]propan-1-ol |
| 66 | 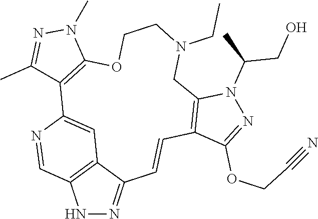 [SMILES: CCN1CCOc2c(c(C)nn2C)-c2cc3c(n[nH]c3cn2)/C=C/c2c(OCC#N)nn([C@@H](C)CO)c2C1] | {[(17E)-12-ethyl-14-[(2S)-1- hydroxypropan-2-yl]-6,8-dimethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 67 | 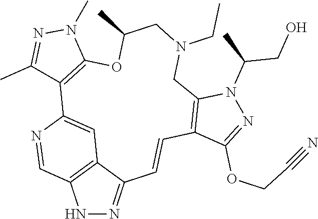 [SMILES: CCN1Cc2c(c(OCC#N)nn2[C@@H](C)CO)/C=C/c2n[nH]c3cnc(cc23)-c2c(C)nn(C)c2O[C@@H](C)C1] | {[(10S,17E)-12-ethyl-14-[(2S)-1- hydroxypropan-2-yl]-6,8,10-trimethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 68 | 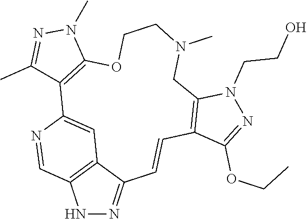 [SMILES: CCOc1nn(CCO)c2c1/C=C/c1n[nH]c3cnc(cc13)-c1c(C)nn(C)c1OCCN(C)C2] | 2-[(17E)-16-ethoxy-6,8,12-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 69 | 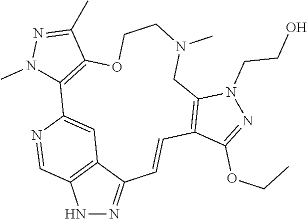 [SMILES: CCOc1nn(CCO)c2c1/C=C/c1n[nH]c3cnc(cc13)-c1c(c(C)nn1C)OCCN(C)C2] | 2-[(17E)-16-ethoxy-6,8,12-trimethyl- 2,6,10,11,12,13-hexahydro-14H-5,3- (azenometheno)tripyrazolo[3,4-f:3′,4′- j:3″,4″-n][1,4]oxazacyclopentadecin- 14-yl]ethan-1-ol |
| 70 | 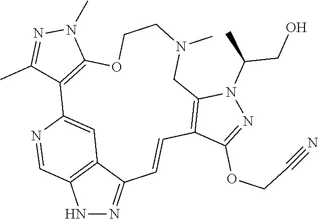 [SMILES: Cc1nn(C)c2c1-c1cc3c(n[nH]c3cn1)/C=C/c1c(OCC#N)nn([C@@H](C)CO)c1CN(C)CCO2] | {[(17E)-14-[(2S)-1-hydroxypropan-2- yl]-6,8,12-trimethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 71 | 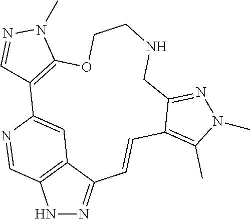 [SMILES: Cc1c2c(nn1C)CNCCOc1c(cnn1C)-c1cc3c(n[nH]c3cn1)/C=C/2] | (17E)-8,15,16-trimethyl- 2,10,11,12,13,15-hexahydro-8H-5,3- (azenometheno)tripyrazolo[3,4-f:3′,4′- j:4″,3″-n][1,4]oxazacyclopentadecine |
| 72 | 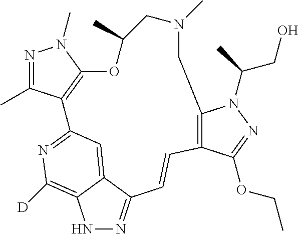 [SMILES: [2H]c1nc2cc3c(n[nH]c13)/C=C/c1c(OCC)nn([C@@H](C)CO)c1CN(C)C[C@H](C)Oc1c-2c(C)nn1C] | (2S)-2-[(10S,17E)-16-ethoxy- 6,8,10,12-tetramethyl(20-2H)- 10,11,12,13-tetrahydro-8H-5,3- (azenometheno)tripyrazolo[3,4-f:3′,4′- j:4″,3″-n][1,4]oxazacyclopentadecin- 14(2H)-yl]propan-1-ol |
| 73 | 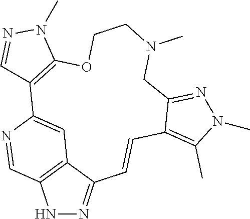 [SMILES: Cc1c2c(nn1C)CN(C)CCOc1c(cnn1C)-c1cc3c(n[nH]c3cn1)/C=C/2] | (17E)-8,12,15,16-tetramethyl- 2,10,11,12,13,15-hexahydro-8H-5,3- (azenometheno)tripyrazolo[3,4-f:3′,4′- j:4″,3″-n][1,4]oxazacyclopentadecine |
| 74 | 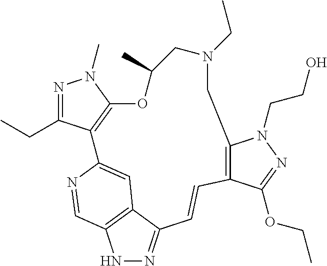 [SMILES: CCOc1nn(CCO)c2c1/C=C/c1n[nH]c3cnc(cc13)-c1c(CC)nn(C)c1O[C@@H](C)CN(CC)C2] | 2-[(10S,17E)-16-ethoxy-6,12-diethyl- 8,10-dimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 75 | 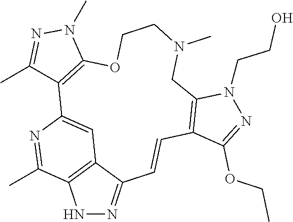 [SMILES: CCOc1nn(CCO)c2c1/C=C/c1n[nH]c3c(C)nc(cc13)-c1c(C)nn(C)c1OCCN(C)C2] | 2-[(17E)-16-ethoxy-6,8,12,20- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 77 | 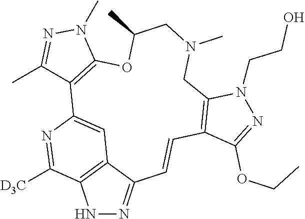 [SMILES: [2H]C([2H])([2H])c1nc2cc3c(n[nH]c13)/C=C/c1c(OCC)nn(CCO)c1CN(C)C[C@H](C)Oc1c-2c(C)nn1C] | 2-[(10S,17E)-16-ethoxy-6,8,10,12- tetramethyl-20-(²H₃)methyl- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 78 | 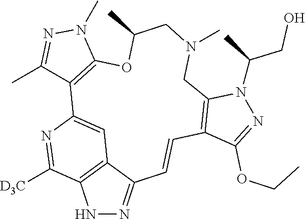 [SMILES: [2H]C([2H])([2H])c1nc2cc3c(n[nH]c13)/C=C/c1c(OCC)nn([C@@H](C)CO)c1CN(C)C[C@H](C)Oc1c-2c(C)nn1C] | (2S)-2-[(10S,17E)-16-ethoxy- 6,8,10,12-tetramethyl-20-(²H₃)methyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 79 | 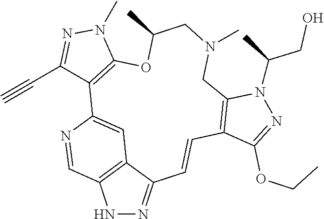 [SMILES: C#Cc1nn(C)c2c1-c1cc3c(n[nH]c3cn1)/C=C/c1c(OCC)nn([C@@H](C)CO)c1CN(C)C[C@H](C)O2] | (2S)-2-[(10S,17E)-16-ethoxy-6- ethynyl-8,10,12-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 80 | 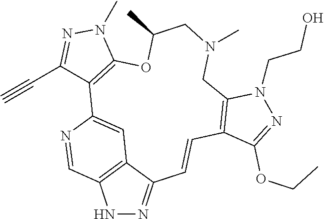 [SMILES: C#Cc1nn(C)c2c1-c1cc3c(n[nH]c3cn1)/C=C/c1c(OCC)nn(CCO)c1CN(C)C[C@H](C)O2] | 2-[(10S,17E)-16-ethoxy-6-ethynyl- 8,10,12-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 81 | 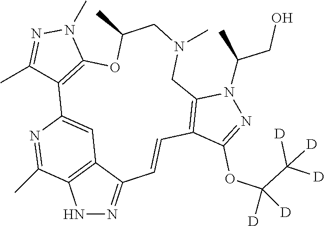 [SMILES: [2H]C([2H])([2H])C([2H])([2H])Oc1nn([C@@H](C)CO)c2c1/C=C/c1n[nH]c3c(C)nc(cc13)-c1c(C)nn(C)c1O[C@@H](C)CN(C)C2] | (2S)-2-[(10S,17E)-16-[(²H₅)ethyloxy]- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 82 | 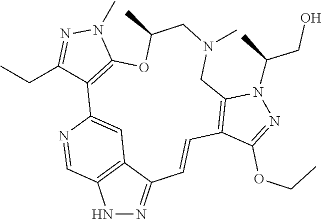 [SMILES: CCOc1nn([C@@H](C)CO)c2c1/C=C/c1n[nH]c3cnc(cc13)-c1c(CC)nn(C)c1O[C@@H](C)CN(C)C2] | (2S)-2-[(10S,17E)-16-ethoxy-6-ethyl- 8,10,12-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 83 | 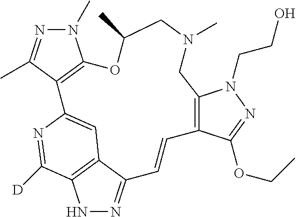 [SMILES: [2H]c1nc2cc3c(n[nH]c13)/C=C/c1c(OCC)nn(CCO)c1CN(C)C[C@H](C)Oc1c-2c(C)nn1C] | 2-[(10S,17E)-16-ethoxy-6,8,10,12- tetramethyl(20-2H)-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 84 | 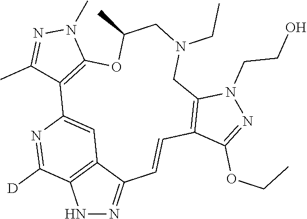 [SMILES: [2H]c1nc2cc3c(n[nH]c13)/C=C/c1c(OCC)nn(CCO)c1CN(CC)C[C@H](C)Oc1c-2c(C)nn1C] | 2-[(10S,17E)-16-ethoxy-12-ethyl- 6,8,10-trimethyl(20-2H)- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 85 | 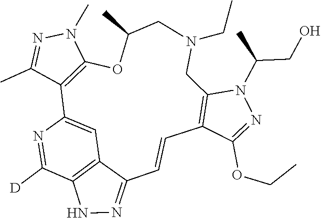 | (2S)-2-[(10S,17E)-16-ethoxy-12-ethyl- 6,8,10-trimethyl(20-2H)- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 86 | 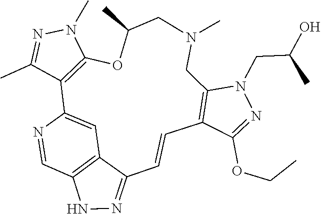 | (2S)-1-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-2-ol |
| 87 | 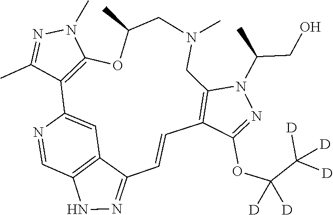 | (2S)-2-[(10S,17E)-16-[(²H₅)ethyloxy]- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 88 | 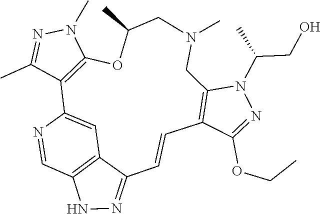 | (2R)-2-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 89 | 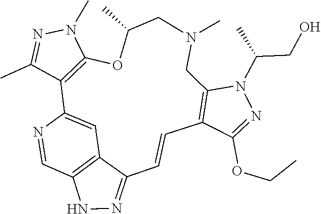 | (2R)-2-[(10R,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 90 | 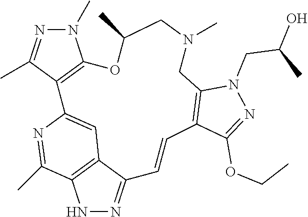 [SMILES: CCOc1nn(C[C@H](C)O)c2c1/C=C/c1n[nH]c3c(C)nc(cc13)-c1c(C)nn(C)c1O[C@@H](C)CN(C)C2] | (2S)-1-[(10S,17E)-16-ethoxy- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-2-ol |
| 91 | 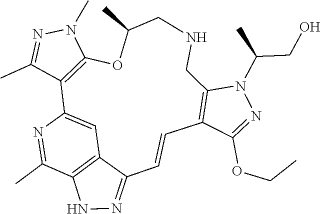 | (2S)-2-[(10S,17E)-16-ethoxy- 6,8,10,20-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 92 | 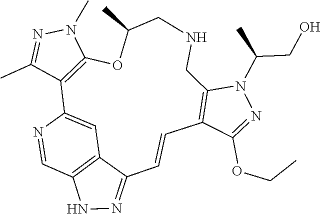 | (2S)-2-[(10S,17E)-16-ethoxy-6,8,10- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]propan-1-ol |
| 93 | 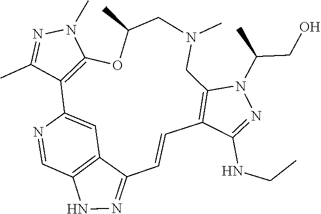 | (2S)-2-[(10S,17E)-16-(ethylamino)- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 94 | 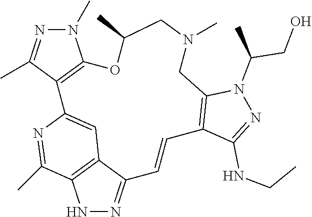 [SMILES: CCNc1nn([C@@H](C)CO)c2c1/C=C/c1n[nH]c3c(C)nc(cc13)-c1c(C)nn(C)c1O[C@@H](C)CN(C)C2] | (2S)-2-[(10S,17E)-16-(ethylamino)- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 95 | 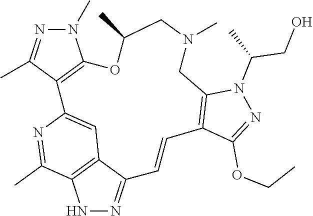 [SMILES: CCOc1nn([C@H](C)CO)c2c1/C=C/c1n[nH]c3c(C)nc(cc13)-c1c(C)nn(C)c1O[C@@H](C)CN(C)C2] | (2R)-2-[(10S,17E)-16-ethoxy- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 96 | 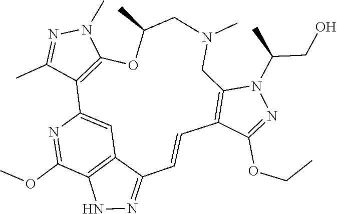 | (2S)-2-[(10S,17E)-16-ethoxy-20- methoxy-6,8,10,12-tetramethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 97 | 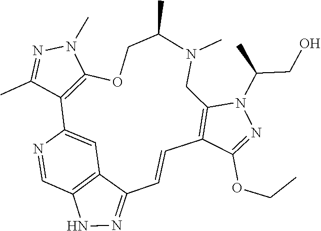 | (2S)-2-[(11R,17E)-16-ethoxy- 6,8,11,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 98 | 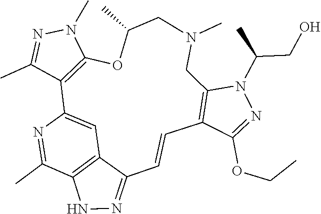 | (2S)-2-[(10R,17E)-16-ethoxy- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 99 | 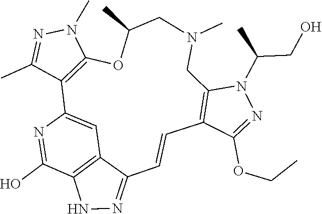 | (10S,17E)-16-ethoxy-14-[(2S)-1- hydroxypropan-2-yl]-6,8,10,12- tetramethyl-2,10,11,12,13,14- hexahydro-8H-5,3- (azenometheno)tripyrazolo[3,4-f:3′,4′- j:4″,3″-n][1,4]oxazacyclopentadecin- 20-ol |
| 100 | 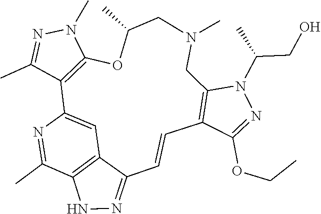 | (2R)-2-[(10R,17E)-16-ethoxy- 6,8,10,12,20-pentamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 101 | 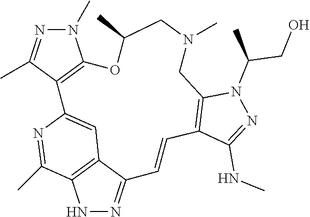 [SMILES: CNc1nn([C@@H](C)CO)c2c1/C=C/c1n[nH]c3c(C)nc(cc13)-c1c(C)nn(C)c1O[C@@H](C)CN(C)C2] | (2S)-2-[(10S,17E)-6,8,10,12,20- pentamethyl-16-(methylamino)- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 102 | 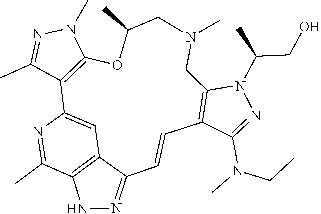 | (2S)-2-[(10S,17E)-16- [ethyl(methyl)amino]-6,8,10,12,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 103 | 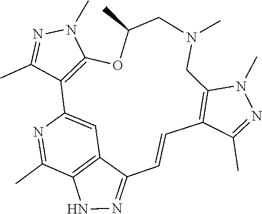 | (10S,17E)-6,8,10,12,14,16,20- heptamethyl-2,10,11,12,13,14- hexahydro-8H-5,3- (azenometheno)tripyrazolo[3,4-f:3′,4′- j:4″,3″-n][1,4]oxazacyclopentadecine |
| 104 | 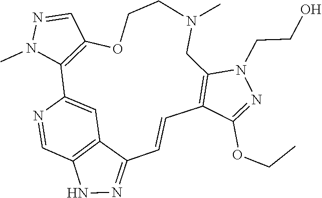 | 2-[(17E)-16-ethoxy-6,12-dimethyl- 2,6,10,11,12,13-hexahydro-14H-5,3- (azenometheno)tripyrazolo[3,4-f:3′,4′- j:3″,4″-n][1,4]oxazacyclopentadecin- 14-yl]ethan-1-ol |
| 105 | 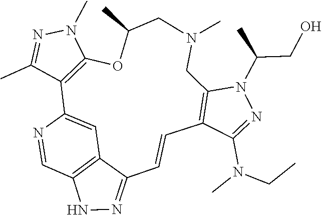 | (2S)-2-[(10S,17E)-16- [ethyl(methyl)amino]-6,8,10,12- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 106 | 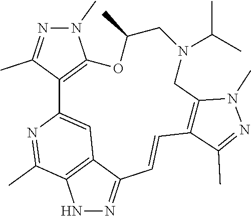 | (10S,17E)-6,8,10,14,16,20-hexamethyl- 12-(propan-2-yl)-2,10,11,12,13,14- hexahydro-8H-5,3- (azenometheno)tripyrazolo[3,4-f:3′,4′- j:4″,3″-n][1,4]oxazacyclopentadecine |
| 107 | 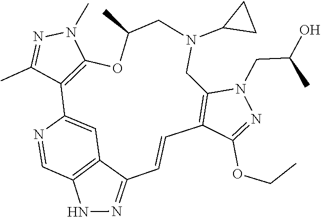 | (2S)-1-[(10S,17E)-12-cyclopropyl-16- ethoxy-6,8,10-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-2-ol |
| 108 | 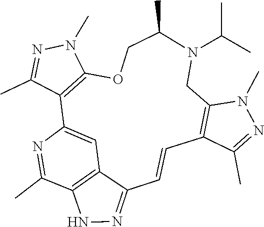 | (11R,17E)-6,8,11,14,16,20- hexamethyl-12-(propan-2-yl)- 2,10,11,12,13,14-hexahydro-8H-5,3- (azenometheno)tripyrazolo[3,4-f:3′,4′- j:4″,3″-n][1,4]oxazacyclopentadecine |
| 109 | 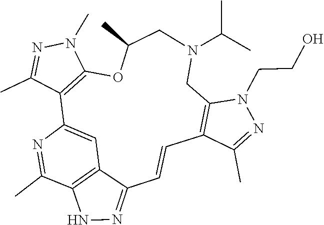 [SMILES: Cc1nn(CCO)c2c1/C=C/c1n[nH]c3c(C)nc(cc13)-c1c(C)nn(C)c1O[C@@H](C)CN(C(C)C)C2] | 2-[(10S,17E)-6,8,10,16,20- pentamethyl-12-(propan-2-yl)- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 110 | 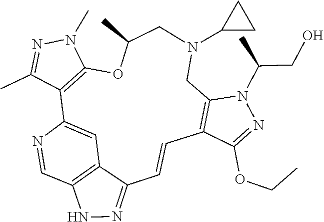 [SMILES: CCOc1nn([C@@H](C)CO)c2c1/C=C/c1n[nH]c3cnc(cc13)-c1c(C)nn(C)c1O[C@@H](C)CN(C1CC1)C2] | (2S)-2-[(10S,17E)-12-cyclopropyl-16- ethoxy-6,8,10-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 111 | 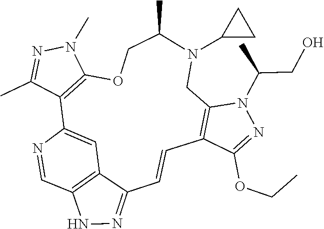 [SMILES: CCOc1nn([C@@H](C)CO)c2c1/C=C/c1n[nH]c3cnc(cc13)-c1c(C)nn(C)c1OC[C@@H](C)N(C1CC1)C2] | (2S)-2-[(11R,17E)-12-cyclopropyl-16- ethoxy-6,8,11-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 112 | 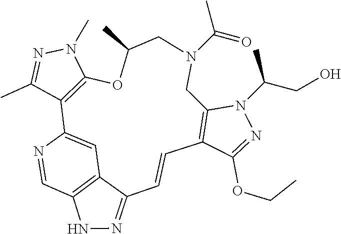 | 1-[(10S,17E)-16-ethoxy-14-[(2S)-1- hydroxypropan-2-yl]-6,8,10-trimethyl- 2,8,10,11,13,14-hexahydro-12H-5,3- (azenometheno)tripyrazolo[3,4-f:3′,4′- j:4″,3″-n][1,4]oxazacyclopentadecin- 12-yl]ethan-1-one |
| 113 | 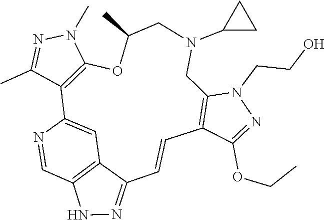 | 2-[(10S,17E)-12-cyclopropyl-16- ethoxy-6,8,10-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 114 | 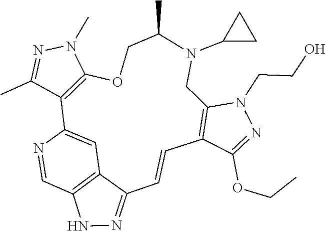 | 2-[(11R,17E)-12-cyclopropyl-16- ethoxy-6,8,11-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]ethan-1-ol |
| 115 | 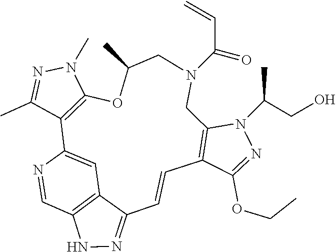 | 1-[(10S,17E)-16-ethoxy-14-[(2S)-1- hydroxypropan-2-yl]-6,8,10-trimethyl- 2,8,10,11,13,14-hexahydro-12H-5,3- (azenometheno)tripyrazolo[3,4-f:3′,4′- j:4″,3″-n][1,4]oxazacyclopentadecin- 12-yl]prop-2-en-1-one |
| 116 | 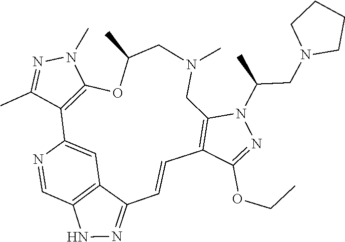 | (10S,17E)-16-ethoxy-6,8,10,12- tetramethyl-14-[(2S)-1-(pyrrolidin-1- yl)propan-2-yl]-2,10,11,12,13,14- hexahydro-8H-5,3- (azenometheno)tripyrazolo[3,4-f:3′,4′- j:4″,3″-n][1,4]oxazacyclopentadecine |
| 117 | 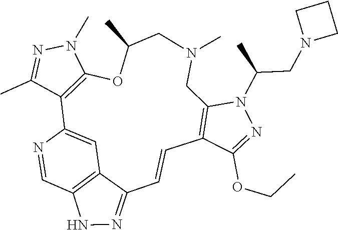 | (10S,17E)-14-[(2S)-1-(azetidin-1- yl)propan-2-yl]-16-ethoxy-6,8,10,12- tetramethyl-2,10,11,12,13,14- hexahydro-8H-5,3- (azenometheno)tripyrazolo[3,4-f:3′,4′- j:4″,3″-n][1,4]oxazacyclopentadecine |
| 118 | 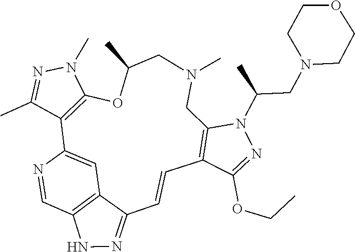 | (10S,17E)-16-ethoxy-6,8,10,12- tetramethyl-14-[(2S)-1-(morpholin-4- yl)propan-2-yl]-2,10,11,12,13,14- hexahydro-8H-5,3- (azenometheno)tripyrazolo[3,4-f:3′,4′- j:4″,3″-n][1,4]oxazacyclopentadecine |
| 119 | 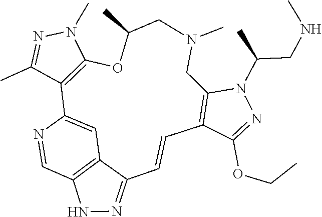 | (2S)-2-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]-N-methylpropan-1-amine |
| 120 | 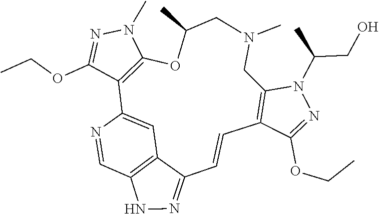 | (2S)-2-[(10S,17E)-6,16-diethoxy- 8,10,12-trimethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 121 | 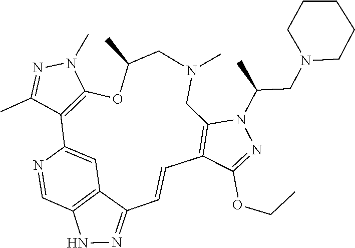 | (10S,17E)-16-ethoxy-6,8,10,12- tetramethyl-14-[(2S)-1-(piperidin-1- yl)propan-2-yl]-2,10,11,12,13,14- hexahydro-8H-5,3- (azenometheno)tripyrazolo[3,4-f:3′,4′- j:4″,3″-n][1,4]oxazacyclopentadecine |
| 122 | 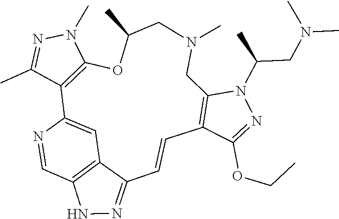 | (2S)-2-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]-N,N-dimethylpropan-1-amine |
| 123 | 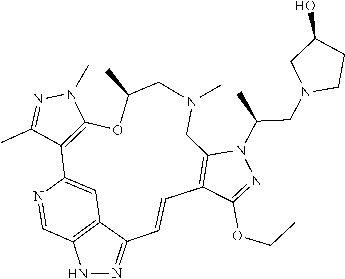 | (3S)-1-{(2S)-2-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propyl}pyrrolidin-3-ol |
| 124 | 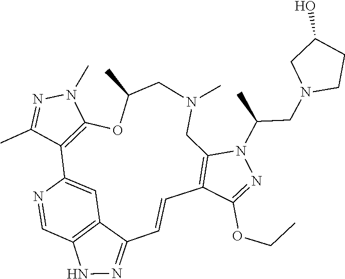 | (3R)-1-{(2S)-2-[(10S,17E)-16-ethoxy- 6,8,10,12-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propyl}pyrrolidin-3-ol |
| 125 | 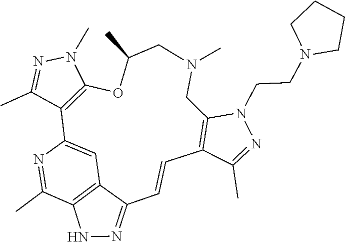 | (10S,17E)-6,8,10,12,16,20-hexamethyl- 14-[2-(pyrrolidin-1-yl)ethyl]- 2,10,11,12,13,14-hexahydro-8H-5,3- (azenometheno)tripyrazolo[3,4-f:3′,4′- j:4″,3″-n][1,4]oxazacyclopentadecine |
| 126 | 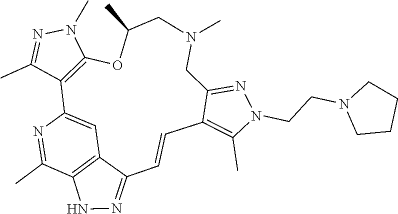 | (10S,17E)-6,8,10,12,16,20-hexamethyl- 15-[2-(pyrrolidin-1-yl)ethyl]- 2,10,11,12,13,15-hexahydro-8H-5,3- (azenometheno)tripyrazolo[3,4-f:3′,4′- j:4″,3″-n][1,4]oxazacyclopentadecine |
| 127 | 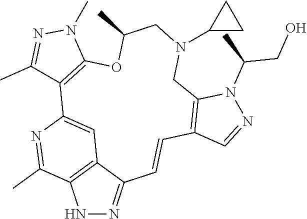 [SMILES: Cc1nn(C)c2c1-c1cc3c(n[nH]c3c(C)n1)/C=C/c1cnn([C@@H](C)CO)c1CN(C1CC1)C[C@H](C)O2] | (2S)-2-[(10S,17E)-12-cyclopropyl- 6,8,10,20-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 128 | 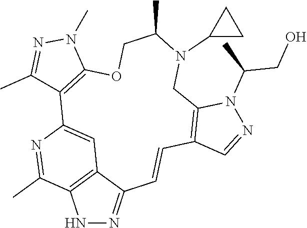 [SMILES: Cc1nn(C)c2c1-c1cc3c(n[nH]c3c(C)n1)/C=C/c1cnn([C@@H](C)CO)c1CN(C1CC1)[C@H](C)CO2] | (2S)-2-[(11R,17E)-12-cyclopropyl- 6,8,11,20-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 129 | 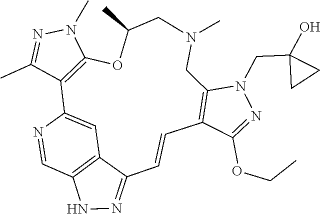 | 1-{[(10S,17E)-16-ethoxy-6,8,10,12- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]methyl}cyclopropan-1-ol |
| 130 | 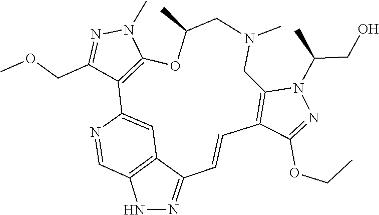 | (2S)-2-[(10S,17E)-16-ethoxy-6- (methoxymethyl)-8,10,12-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 131 | 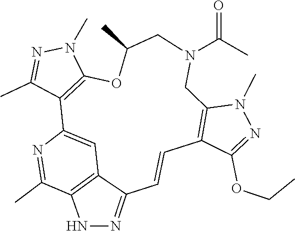 [SMILES: CCOc1nn(C)c2c1/C=C/c1n[nH]c3c(C)nc(cc13)-c1c(C)nn(C)c1O[C@@H](C)CN(C(C)=O)C2] | 1-[(10S,17E)-16-ethoxy-6,8,10,14,20- pentamethyl-2,8,10,11,13,14- hexahydro-12H-5,3- (azenometheno)tripyrazolo[3,4-f:3′,4′- j:4″,3″-n][1,4]oxazacyclopentadecin- 12-yl]ethan-1-one |
| 132a | 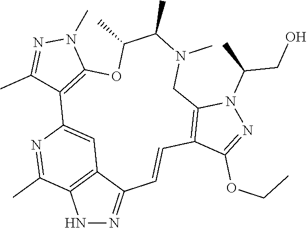 [SMILES: CCOc1nn([C@@H](C)CO)c2c1/C=C/c1n[nH]c3c(C)nc(cc13)-c1c(C)nn(C)c1O[C@H](C)[C@@H](C)N(C)C2] | (2S)-2-[(10R,11R,17E)-16-ethoxy- 6,8,10,11,12,20-hexamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 132b | 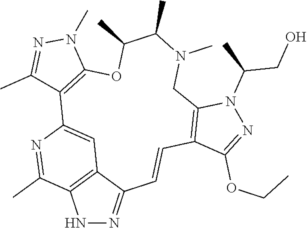 [SMILES: CCOc1nn([C@@H](C)CO)c2c1/C=C/c1n[nH]c3c(C)nc(cc13)-c1c(C)nn(C)c1O[C@@H](C)[C@@H](C)N(C)C2] | (2S)-2-[(10S,11R,17E)-16-ethoxy- 6,8,10,11,12,20-hexamethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 133 | 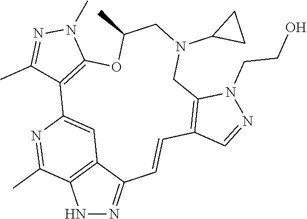 [SMILES: Cc1nn(C)c2c1-c1cc3c(n[nH]c3c(C)n1)/C=C/c1cnn(CCO)c1CN(C1CC1)C[C@H](C)O2] | 2-[(10S,17E)-12-cyclopropyl- 6,8,10,20-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 134 | 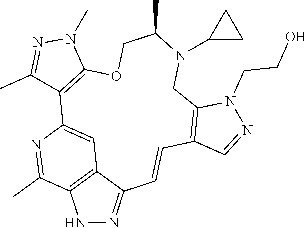 [SMILES: Cc1nn(C)c2c1-c1cc3c(n[nH]c3c(C)n1)/C=C/c1cnn(CCO)c1CN(C1CC1)[C@H](C)CO2] | 2-[(11R,17E)-12-cyclopropyl- 6,8,11,20-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 135 | 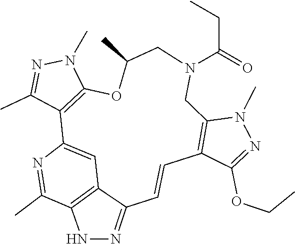 [SMILES: CCOc1nn(C)c2c1/C=C/c1n[nH]c3c(C)nc(cc13)-c1c(C)nn(C)c1O[C@@H](C)CN(C(=O)CC)C2] | 1-[(10S,17E)-16-ethoxy-6,8,10,14,20- pentamethyl-2,8,10,11,13,14- hexahydro-12H-5,3- (azenometheno)tripyrazolo[3,4-f:3′,4′- j:4″,3″-n][1,4]oxazacyclopentadecin- 12-yl]propan-1-one |
| 136 | 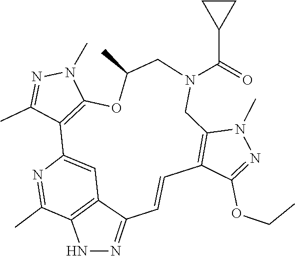 [SMILES: CCOc1nn(C)c2c1/C=C/c1n[nH]c3c(C)nc(cc13)-c1c(C)nn(C)c1O[C@@H](C)CN(C(=O)C1CC1)C2] | cyclopropyl[(10S,17E)-16-ethoxy- 6,8,10,14,20-pentamethyl- 2,8,10,11,13,14-hexahydro-12H-5,3- (azenometheno)tripyrazolo[3,4-f:3′,4′- j:4″,3″-n][1,4]oxazacyclopentadecin- 12-yl]methanone |
| 137 | 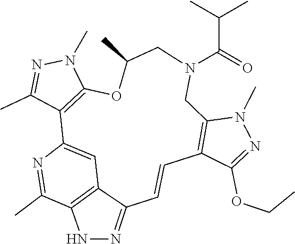 [SMILES: CCOc1nn(C)c2c1/C=C/c1n[nH]c3c(C)nc(cc13)-c1c(C)nn(C)c1O[C@@H](C)CN(C(=O)C(C)C)C2] | 1-[(10S,17E)-16-ethoxy-6,8,10,14,20- pentamethyl-2,8,10,11,13,14- hexahydro-12H-5,3- (azenometheno)tripyrazolo[3,4-f:3′,4′- j:4″,3″-n][1,4]oxazacyclopentadecin- 12-yl]-2-methylpropan-1-one |
| 138 | 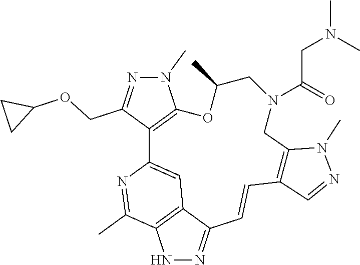 [SMILES: Cc1nc2cc3c(n[nH]c13)/C=C/c1cnn(C)c1CN(C(=O)CN(C)C)C[C@H](C)Oc1c-2c(COC2CC2)nn1C] | 1-[(10S,17E)-6- [(cyclopropyloxy)methyl]-8,10,14,20- tetramethyl-2,8,10,11,13,14- hexahydro-12H-5,3- (azenometheno)tripyrazolo[3,4-f:3′,4′- j:4″,3″-n][1,4]oxazacyclopentadecin- 12-yl]-2-(dimethylamino)ethan-1-one |
| 139 | 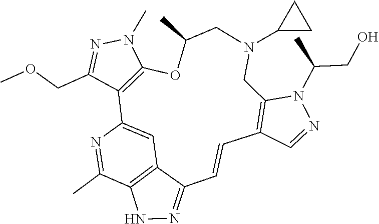 | (2S)-2-[(10S,17E)-12-cyclopropyl-6- (methoxymethyl)-8,10,20-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 140 | 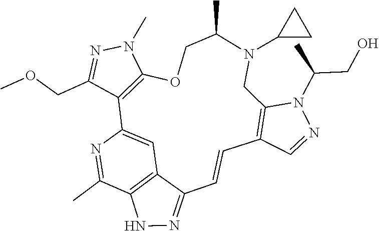 | (2S)-2-[(11R,17E)-12-cyclopropyl-6- (methoxymethyl)-8,11,20-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 141 | 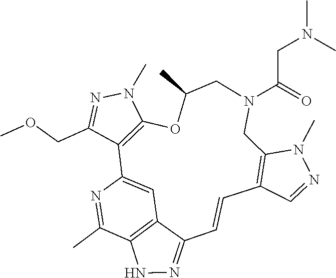 | 2-(dimethylamino)-1-[(10S,17E)-6- (methoxymethyl)-8,10,14,20- tetramethyl-2,8,10,11,13,14- hexahydro-12H-5,3- (azenometheno)tripyrazolo[3,4-f:3′,4′- j:4″,3″-n][1,4]oxazacyclopentadecin- 12-yl]ethan-1-one |
| 142 | 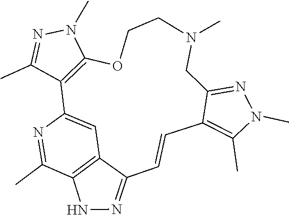 | (17E)-6,8,12,15,16,20-hexamethyl- 2,10,11,12,13,15-hexahydro-8H-5,3- (azenometheno)tripyrazolo[3,4-f:3′,4′- j:4″,3″-n][1,4]oxazacyclopentadecine |
| 143 | 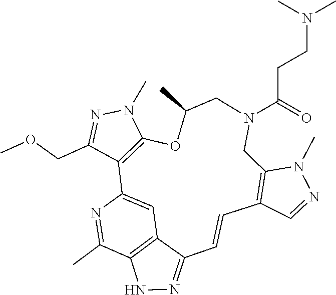 | 3-(dimethylamino)-1-[(10S,17E)-6- (methoxymethyl)-8,10,14,20- tetramethyl-2,8,10,11,13,14- hexahydro-12H-5,3- (azenometheno)tripyrazolo[3,4-f:3′,4′- j:4″,3″-n][1,4]oxazacyclopentadecin- 12-yl]propan-1-one |
| 144 | 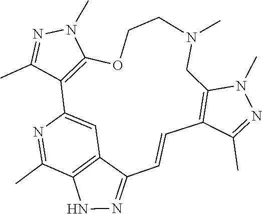 | (17E)-6,8,12,14,16,20-hexamethyl- 2,10,11,12,13,14-hexahydro-8H-5,3- (azenometheno)tripyrazolo[3,4-f:3′,4′- j:4″,3″-n][1,4]oxazacyclopentadecine |
| 145 | 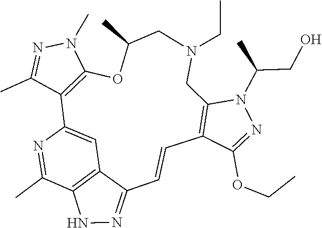 | (2S)-2-[(10S,17E)-16-ethoxy-12-ethyl- 6,8,10,20-tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 146 | 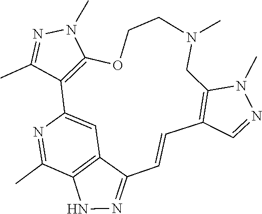 | (17E)-6,8,12,14,20-pentamethyl- 2,10,11,12,13,14-hexahydro-8H-5,3- (azenometheno)tripyrazolo[3,4-f:3′,4′- j:4″,3″-n][1,4]oxazacyclopentadecine |
| 147 | 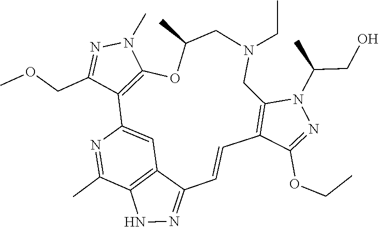 | (2S)-2-[(10S,17E)-16-ethoxy-12-ethyl- 6-(methoxymethyl)-8,10,20-trimethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 148 | 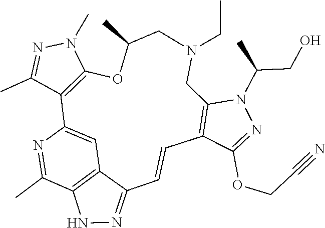 | {[(10S,17E)-12-ethyl-14-[(2S)-1- hydroxypropan-2-yl]-6,8,10,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 149 | 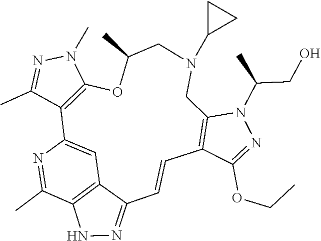 | (2S)-2-[(10S,17E)-12-cyclopropyl-16- ethoxy-6,8,10,20-tetramethyl- 2,8,10,11,12,13-hexahydro-14H-5,3- (azenometheno)tripyrazolo[3,4-f:3′,4′- j:4″,3″-n][1,4]oxazacyclopentadecin- 14-yl]propan-1-ol |
| 150 | 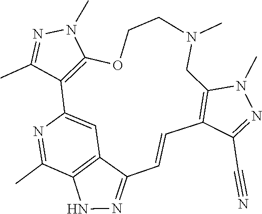 | (17E)-6,8,12,14,20-pentamethyl- 2,10,11,12,13,14-hexahydro-8H-5,3- (azenometheno)tripyrazolo[3,4-f:3′,4′- j:4″,3″-n][1,4]oxazacyclopentadecine- 16-carbonitrile |
| 151 | 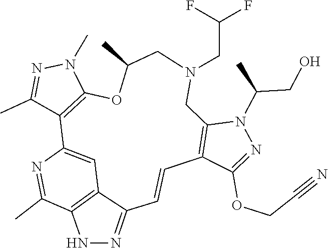 [SMILES: Cc1nn(C)c2c1-c1cc3c(n[nH]c3c(C)n1)/C=C/c1c(OCC#N)nn([C@@H](C)CO)c1CN(CC(F)F)C[C@H](C)O2] | {[(10S,17E)-12-(2,2-difluoroethyl)-14- [(2S)-1-hydroxypropan-2-yl]-6,8,10,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 152 | 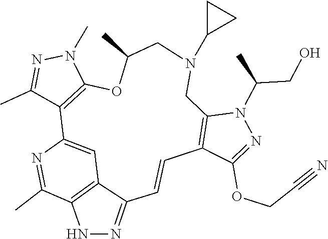 [SMILES: Cc1nn(C)c2c1-c1cc3c(n[nH]c3c(C)n1)/C=C/c1c(OCC#N)nn([C@@H](C)CO)c1CN(C1CC1)C[C@H](C)O2] | {[(10S,17E)-12-cyclopropyl-14-[(2S)- 1-hydroxypropan-2-yl]-6,8,10,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 153 | 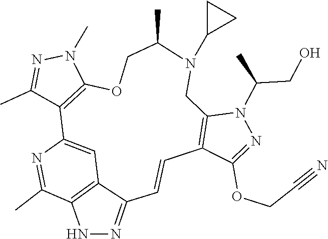 [SMILES: Cc1nn(C)c2c1-c1cc3c(n[nH]c3c(C)n1)/C=C/c1c(OCC#N)nn([C@@H](C)CO)c1CN(C1CC1)[C@H](C)CO2] | {[(11R,17E)-12-cyclopropyl-14-[(2S)- 1-hydroxypropan-2-yl]-6,8,11,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 154 | 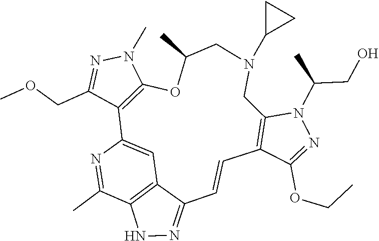 | (2S)-2-[(10S,17E)-12-cyclopropyl-16- ethoxy-6-(methoxymethyl)-8,10,20- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]propan-1-ol |
| 155 | 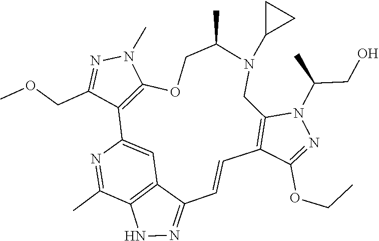 | (2S)-2-[(11R,17E)-12-cyclopropyl-16- ethoxy-6-(methoxymethyl)-8,11,20- trimethyl-2,8,10,11,12,13-hexahydro- 14H-5,3- (azenometheno)tripyrazolo[3,4-f:3′,4′- j:4″,3″-n][1,4]oxazacyclopentadecin- 14-yl]propan-1-ol |
| 156 | 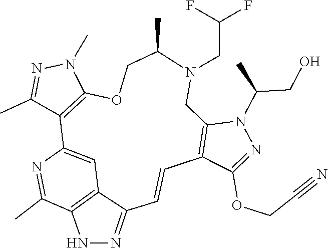 [SMILES: Cc1nn(C)c2c1-c1cc3c(n[nH]c3c(C)n1)/C=C/c1c(OCC#N)nn([C@@H](C)CO)c1CN(CC(F)F)[C@H](C)CO2] | {[(11R,17E)-12-(2,2-difluoroethyl)-14- [(2S)-1-hydroxypropan-2-yl]-6,8,11,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 157 | 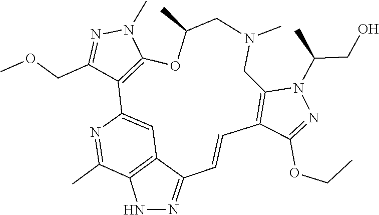 | (2S)-2-[(10S,17E)-16-ethoxy-6- (methoxymethyl)-8,10,12,20- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 158 | 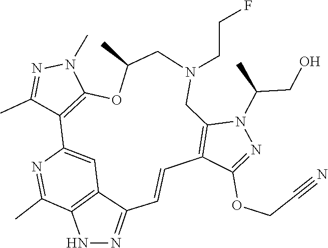 [SMILES: Cc1nn(C)c2c1-c1cc3c(n[nH]c3c(C)n1)/C=C/c1c(OCC#N)nn([C@@H](C)CO)c1CN(CCF)C[C@H](C)O2] | {[(10S,17E)-12-(2-fluoroethyl)-14- [(2S)-1-hydroxypropan-2-yl]-6,8,10,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 159 | 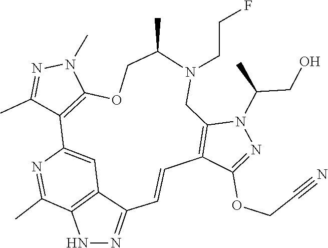 [SMILES: Cc1nn(C)c2c1-c1cc3c(n[nH]c3c(C)n1)/C=C/c1c(OCC#N)nn([C@@H](C)CO)c1CN(CCF)[C@H](C)CO2] | {[(11R,17E)-12-(2-fluoroethyl)-14- [(2S)-1-hydroxypropan-2-yl]-6,8,11,20- tetramethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 160 | 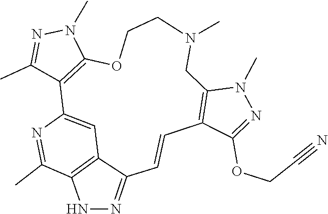 [SMILES: Cc1nn(C)c2c1-c1cc3c(n[nH]c3c(C)n1)/C=C/c1c(OCC#N)nn(C)c1CN(C)CCO2] | {[(17E)-6,8,12,14,20-pentamethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 161 | 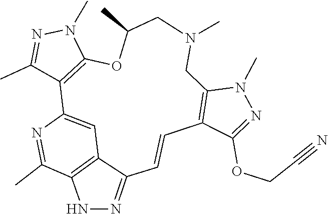 [SMILES: Cc1nn(C)c2c1-c1cc3c(n[nH]c3c(C)n1)/C=C/c1c(OCC#N)nn(C)c1CN(C)C[C@H](C)O2] | {[(10S,17E)-6,8,10,12,14,20- hexamethyl-2,10,11,12,13,14- hexahydro-8H-5,3- (azenometheno)tripyrazolo[3,4-f:3′,4′- j:4″,3″-n][1,4]oxazacyclopentadecin- 16-yl]oxy}acetonitrile |
| 162 | 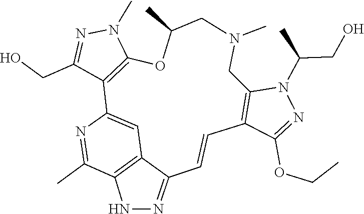 | (2S)-2-[(10S,17E)-16-ethoxy-6- (hydroxymethyl)-8,10,12,20- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 163 | 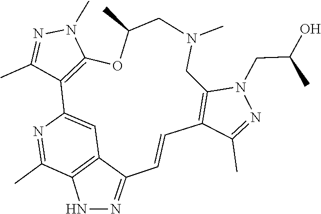 | (2S)-1-[(10S,17E)-6,8,10,12,16,20- hexamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-2-ol |
| 164 | 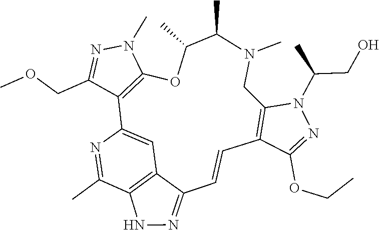 | (2S)-2-[(10R,11R,17E)-16-ethoxy-6- (methoxymethyl)-8,10,11,12,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 165 | 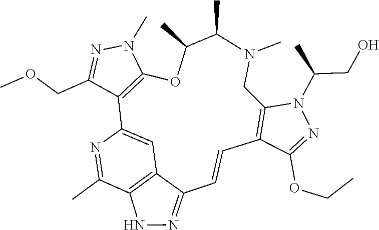 | (2S)-2-[(10S,11R,17E)-16-ethoxy-6- (methoxymethyl)-8,10,11,12,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-1-ol |
| 166 | 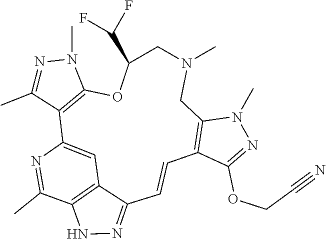 [SMILES: Cc1nn(C)c2c1-c1cc3c(n[nH]c3c(C)n1)/C=C/c1c(OCC#N)nn(C)c1CN(C)C[C@H](C(F)F)O2] | {[(10R,17E)-10-(difluoromethyl)- 6,8,12,14,20-pentamethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 167 | 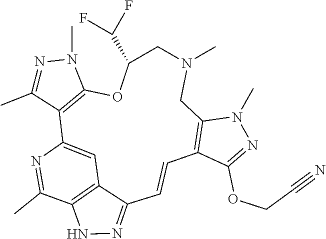 [SMILES: Cc1nn(C)c2c1-c1cc3c(n[nH]c3c(C)n1)/C=C/c1c(OCC#N)nn(C)c1CN(C)C[C@@H](C(F)F)O2] | {[(10S,17E)-10-(difluoromethyl)- 6,8,12,14,20-pentamethyl- 2,10,11,12,13,14-hexahydro-8H-5,3- (azenometheno)tripyrazolo[3,4-f:3′,4′- j:4″,3″-n][1,4]oxazacyclopentadecin- 16-yl]oxy}acetonitrile |
| 168 | 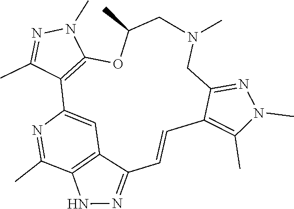 | (10S,17E)-6,8,10,12,15,16,20- heptamethyl-2,10,11,12,13,15- hexahydro-8H-5,3- (azenometheno)tripyrazolo[3,4-f:3′,4′- j:4″,3″-n][1,4]oxazacyclopentadecine |
| 169 | 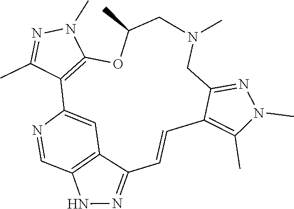 | (10S,17E)-6,8,10,12,15,16-hexamethyl- 2,10,11,12,13,15-hexahydro-8H-5,3- (azenometheno)tripyrazolo[3,4-f:3′,4′- j:4″,3″-n][1,4]oxazacyclopentadecine |
| 170 | 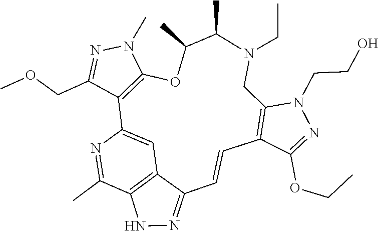 | 2-[(10S,11R,17E)-16-ethoxy-12-ethyl- 6-(methoxymethyl)-8,10,11,20- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 171 | 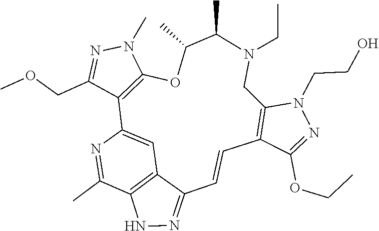 | 2-[(10R,11R,17E)-16-ethoxy-12-ethyl- 6-(methoxymethyl)-8,10,11,20- tetramethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 172 | 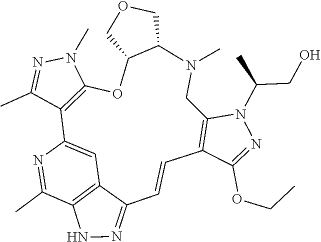 | (2S)-2-[(4aS,7aS,13E)-12-ethoxy- 1,3,8,21-tetramethyl-3,4a,5,7,7a,8,9,16- octahydro-10H-19,17- (azenometheno)furo[3,4- b]tripyrazolo[3,4-f:3′,4′-j:4″,3″- n][1,4]oxazacyclopentadecin-10- yl]propan-1-ol |
| 173 | 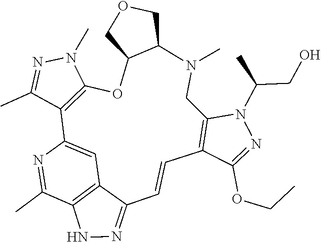 | (2S)-2-[(4aR,7aR,13E)-12-ethoxy- 1,3,8,21-tetramethyl-3,4a,5,7,7a,8,9,16- octahydro-10H-19,17- (azenometheno)furo[3,4- b]tripyrazolo[3,4-f:3′,4′-j:4″,3″- n][1,4]oxazacyclopentadecin-10- yl]propan-1-ol |
| 174 | 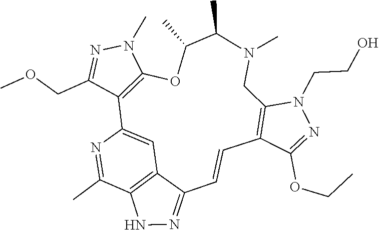 | 2-[(10R,11R,17E)-16-ethoxy-6- (methoxymethyl)-8,10,11,12,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 175 | 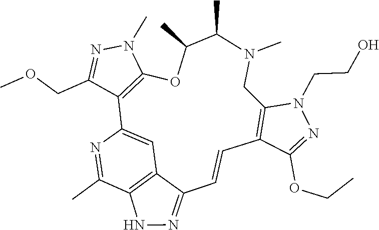 | 2-[(10S,11R,17E)-16-ethoxy-6- (methoxymethyl)-8,10,11,12,20- pentamethyl-2,8,10,11,12,13- hexahydro-14H-5,3- (azenometheno)tripyrazolo[3,4-f:3′,4′- j:4″,3″-n][1,4]oxazacyclopentadecin- 14-yl]ethan-1-ol |
| 176 | 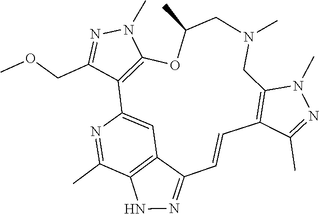 | (10S,17E)-6-(methoxymethyl)- 8,10,12,14,16,20-hexamethyl- 2,10,11,12,13,14-hexahydro-8H-5,3- (azenometheno)tripyrazolo[3,4-f:3′,4′- j:4″,3″-n][1,4]oxazacyclopentadecine |
| 177 | 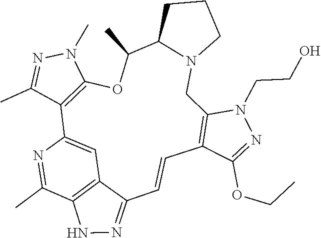 | 2-[(8aR,9S,19E)-1-ethoxy-9,11,13,21- tetramethyl-7,8,8a,9,11,17-hexahydro- 6H-14,16- (azenometheno)tripyrazolo[3,4-f:3′,4′- j:4″,3″-n]pyrrolo[2,1- c][1,4]oxazacyclopentadecin-3(4H)- yl]ethan-1-ol |
| 178 | 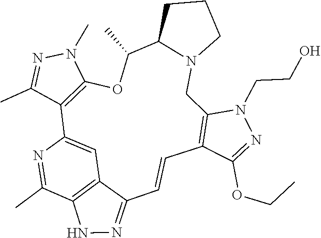 | 2-[(8aR,9R,19E)-1-ethoxy-9,11,13,21- tetramethyl-7,8,8a,9,11,17-hexahydro- 6H-14,16- (azenometheno)tripyrazolo[3,4-f:3′,4′- j:4″,3″-n]pyrrolo[2,1- c][1,4]oxazacyclopentadecin-3(4H)- yl]ethan-1-ol |
| 179 | 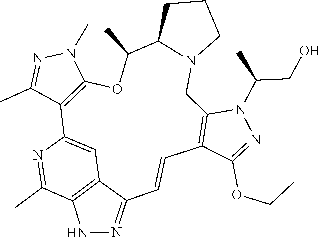 | (2S)-2-[(8aR,9S,19E)-1-ethoxy- 9,11,13,21-tetramethyl-7,8,8a,9,11,17- hexahydro-6H-14,16- (azenometheno)tripyrazolo[3,4-f:3′,4′- j:4″,3″-n]pyrrolo[2,1- c][1,4]oxazacyclopentadecin-3(4H)- yl]propan-1-ol |
| 180 | 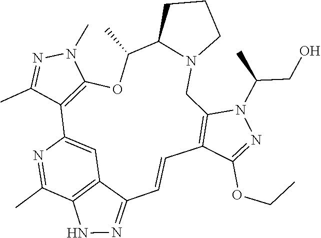 | (2S)-2-[(8aR,9R,19E)-1-ethoxy- 9,11,13,21-tetramethyl-7,8,8a,9,11,17- hexahydro-6H-14,16- (azenometheno)tripyrazolo[3,4-f:3′,4′- j:4″,3″-n]pyrrolo[2,1- c][1,4]oxazacyclopentadecin-3(4H)- yl]propan-1-ol |
| 181 | 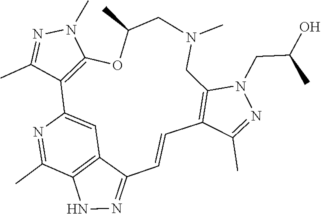 | (2S)-1-[(10S,17E)-6,8,10,12,16,20- hexamethyl-2,8,10,11,12,13- hexahydro-14H-5,3- (azenometheno)tripyrazolo[3,4-f:3′,4′- j:4″,3″-n][1,4]oxazacyclopentadecin- 14-yl]propan-2-ol |

and pharmaceutically acceptable salts, solvates, hydrates, or cocrystals thereof.

Those skilled in the art will recognize that the species listed or illustrated herein are not exhaustive, and that additional species within the scope of these defined terms may also be selected.

### Alternative Embodiments

In some embodiments, the disclosure provides compounds or methods according to the following embodiments.

1. A compound of the formula I

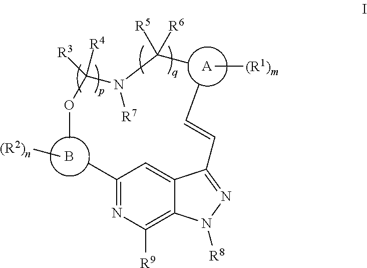

wherein
ring A and ring B are each independently a 5-membered heteroarylene;
each R¹ and R², when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5-to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{d}, —C(O)OR^{d}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R³, R⁴, R⁵, and R⁶ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5-to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or two of R³, R⁴, R⁵, and R⁶, taken together with the carbon or carbons to which they are attached, combine to form C₃-C₆ cycloalkyl or 3- to 7-membered heterocycloalkyl, wherein each hydrogen atom in C₃-C₆ cycloalkyl or 3- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{c}R^{f}, —P(O)₂R^{c}R^{f}, —P(O)NR^{e}R^{f}, —P(O)₂NR^{e}R^{f}, —P(O)OR^{c}, —P(O)₂OR^{c}, —CN, or —NO₂;
R⁷ is H, deuterium, C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, or 5- to 10-membered heteroaryl is independently optionally substituted by —OR^{c}, —OC(O)R^{d}, —OC(O)NR^{c}R^{d}, —OC(═N)NR^{e}R^{d}, —OS(O)R^{e}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{e})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{e}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂; or R⁷ and one of R³, R⁴, R⁵, or R⁶, taken together with the atoms to which each is attached, combine to form a 4- to 7-membered heterocycloalkyl, wherein each hydrogen atom in the 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁸ is H, —S(O)₂R^{c}, —S(O)₂NR^{c}R^{d}, —C(O)R^{c}, —C(O)OR^{c}, —C(O)NR^{c}R^{d}, —C(═N)NR^{c}R^{d}, —P(O)₂R^{c}R^{d}, —P(O)₂NR^{c}R^{d}, or —P(O)₂OR^{c};
R⁹ is H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NRR^{d}, —NR^{c}C(O)R^{d}, —N(C(O)R)(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{e}, —C(O)NR^{c}R^{d}, —C—(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl; or R^{a} and R^{b} or R^{c} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, or 3;
n is 0, 1, 2, or 3;
p is 1, 2, 3, or 4; and
q is 1, 2, or 3;
or a pharmaceutically acceptable salt thereof.

2. The compound of embodiment 1, or a pharmaceutically acceptable salt thereof, having the formula II

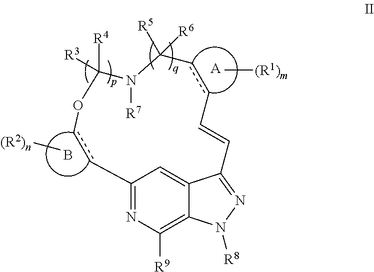

wherein each “ [symbol] ” is independently a carbon-carbon single bond or a carbon-carbon double bond.

3. The compound of embodiment 1 or 2, or a pharmaceutically acceptable salt thereof, having the formula III

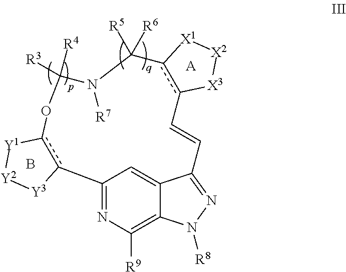

wherein
wherein each “ [symbol] ” is independently a carbon-carbon single bond or a carbon-carbon double bond;
X¹, X², and X³ are each independently —O—, —S—, ═C(H)—, ═C(R¹)—, —N(H)—, —N(R¹)— or ═N— and ring A is a 5-membered heteroarylene, provided that at least one of X¹, X², and X³ is not ═C(H)—, or ═C(R¹)—; and/or
Y¹, Y², and Y³ are each independently —O—, —S—, ═C(H)—, ═C(R²)—, —N(H)—, —N(R²)— or ═N— and ring B is a 5-membered heteroarylene, provided that at least one of Y¹, Y², and Y³ is not ═C(H)—, or ═C(R²)—.

4. The compound of embodiment 3, having the formula IV

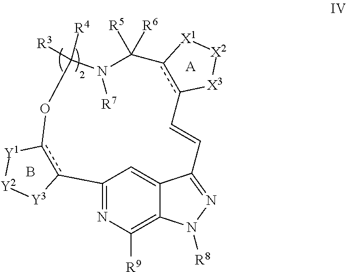

or a pharmaceutically acceptable salt thereof.

5. The compound of embodiment 3 or 4, wherein
X² is ═N— or —N(R¹)—, X¹ and X³ are each independently —O—, —S—, ═C(H)—, ═C(R¹)—, —N(H)—, or —N(R¹)—, and ring A is a 5-membered heteroarylene; and/or
Y² is ═N—, Y¹ and Y³ are each independently —O—, —S—, ═C(H)—, ═C(R²)—, —N(H)—, or —N(R²)—, and ring B is a 5-membered heteroarylene.

6. The compound of any one of embodiments 1 to 4, or a pharmaceutically acceptable salt thereof, wherein:
ring A is selected from the group consisting of

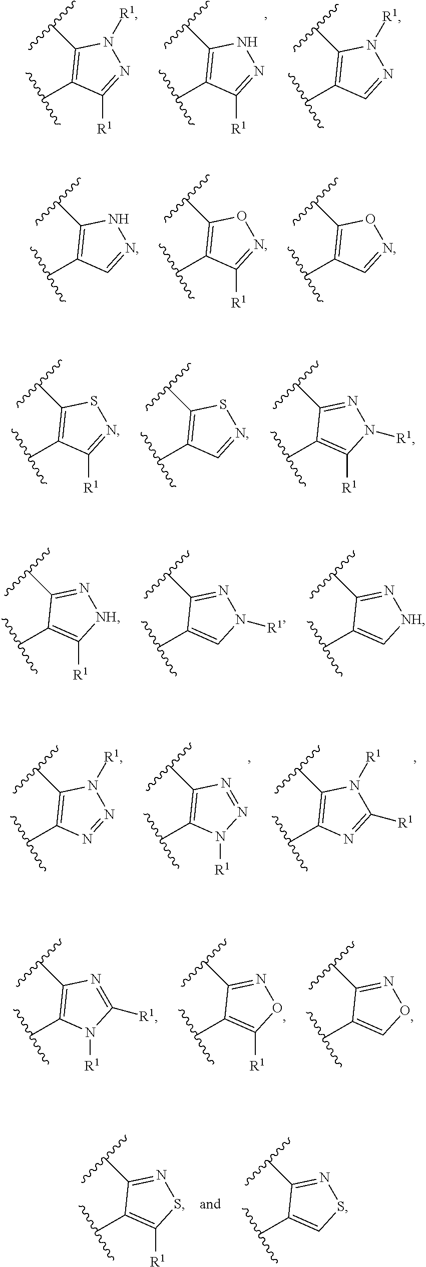

wherein each “ [symbol] ” represents a point of covalent attachment.

7. The compound of any one of the preceding embodiments, or a pharmaceutically acceptable salt thereof, wherein ring A is selected from the group consisting of

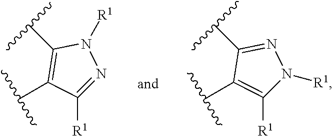

wherein each “ [symbol] ” represents a point of covalent attachment.

8. The compound of any one of the preceding embodiments, or a pharmaceutically acceptable salt thereof, wherein ring A is selected from the group consisting of

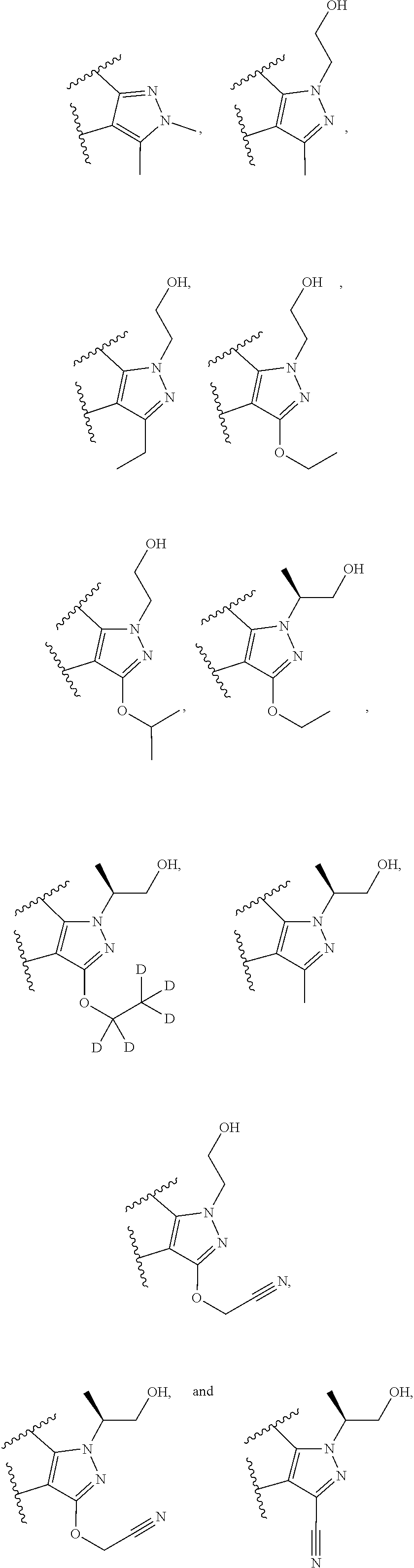

wherein each “ [symbol] ” represents a point of covalent attachment.

9. The compound of any one of embodiments 1 to 4 or 6, or a pharmaceutically acceptable salt thereof, wherein ring B is selected from the group consisting of

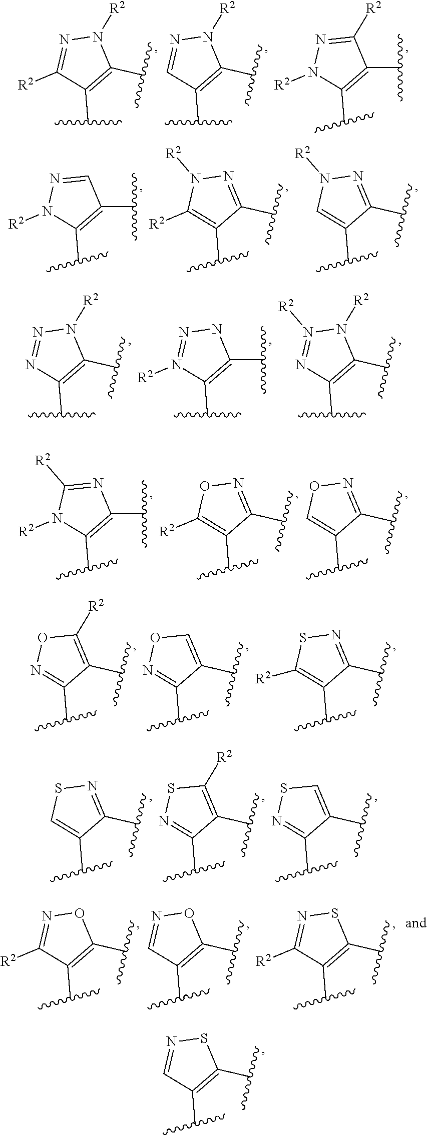

wherein each “ [symbol] ” represents a point of covalent attachment.

10. The compound of any one of the preceding embodiments, or a pharmaceutically acceptable salt thereof, wherein ring B is selected from the group consisting of

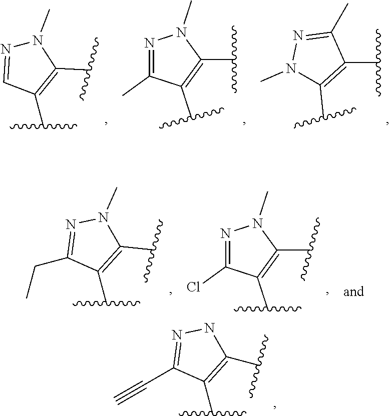

wherein each “ [symbol] ” represents a point of covalent attachment.

11. The compound of any one of the preceding embodiments, or a pharmaceutically acceptable salt thereof, wherein p is 2 or 3, and the portion

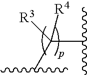

is an ethylene or propylene, wherein each R³ and R⁴ is independently H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or at least one instance of R³ and R⁴, taken together with the carbon or carbons to which they are attached, combine to form C₃-C₆ cycloalkyl or 4- to 7-membered heterocycloalkyl, wherein each hydrogen atom in C₃-C₆ cycloalkyl or 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NRC(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R⁷ and one instance of R³ or R⁴, taken together with the atoms to which they are attached, combine to form 4- to 7-membered heterocycloalkyl, wherein each hydrogen atom in the 4- to 7-membered heterocycloalkyl formed is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and each “ [symbol] ” represents a point of covalent attachment.

12. The compound of any one of the preceding embodiments, or a pharmaceutically acceptable salt thereof, wherein one R³ is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R″, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or two R³ and R⁴, taken together with the carbon or carbons to which they are attached, combine to form C₃-C₆ cycloalkyl or 4- to 7-membered heterocycloalkyl, wherein each hydrogen atom in the C₃-C₆ cycloalkyl or 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or R⁷ and one instance of R³ or R⁴ and, taken together with the atoms to which they are attached, combine to form a 4- to 7-membered heterocycloalkyl, wherein each hydrogen atom in the 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and any remaining R³ and R⁴ are H or deuterium.

13. The compound of any one of the preceding embodiments, or a pharmaceutically acceptable salt thereof, wherein one R³ is C₁-C₆ alkyl, wherein each hydrogen atom in C₁-C₆ alkyl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or R⁷ and one instance of R³ or R⁴ taken together with the atoms to which they are attached, combine to form 4- to 7-membered heterocycloalkyl, wherein each hydrogen atom in the 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and any remaining R³ and R⁴ are H or deuterium.

14. The compound of any one of the preceding embodiments, or a pharmaceutically acceptable salt thereof, wherein one instance of R³ is C₁-C₆ alkyl; and any remaining R³ and R⁴ are H or deuterium.

15. The compound of any one of the preceding embodiments, or a pharmaceutically acceptable salt thereof, wherein one instance of R³ is C₁-C₆ alkyl; R⁷ and one instance of R⁴, taken together with the atoms to which they are attached, combine to form a 4- to 7-membered heterocycloalkyl; and any remaining R³ and R⁴ are H or deuterium.

16. The compound of any one of the preceding embodiments, or a pharmaceutically acceptable salt thereof, wherein one instance of R³ is methyl, and any remaining R³ and R⁴ are H or deuterium.

17. The compound of any one of the preceding embodiments, or a pharmaceutically acceptable salt thereof, wherein R⁷ is H, C₁-C₆ alkyl, or C₃-C₆ cycloalkyl; or R⁷ and one instance of R³ or R⁴, taken together with the atoms to which they are attached, combine to form 4- to 7-membered heterocycloalkyl.

18. The compound of any one of the preceding embodiments, or a pharmaceutically acceptable salt thereof, wherein the portion

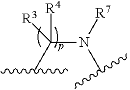

is of the formula

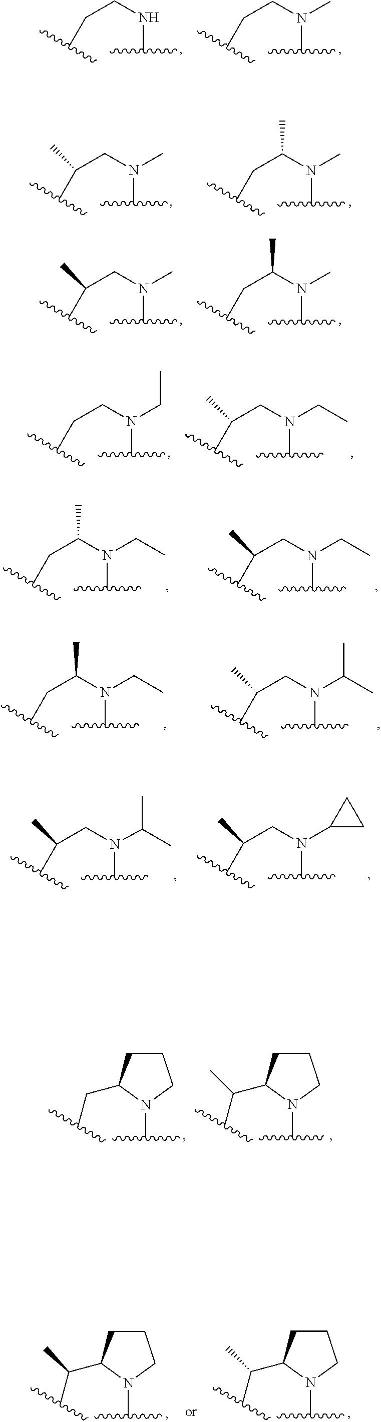

wherein each “ [symbol] ” represents a point of covalent attachment, and each hydrogen is independently optionally substituted with deuterium.

19. The compound of any one of the preceding embodiments, or a pharmaceutically acceptable salt thereof, wherein R⁵ and R⁶ are each H.

20. The compound of any one of the preceding embodiments, or a pharmaceutically acceptable salt thereof, wherein the portion

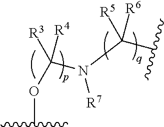

is of the formula

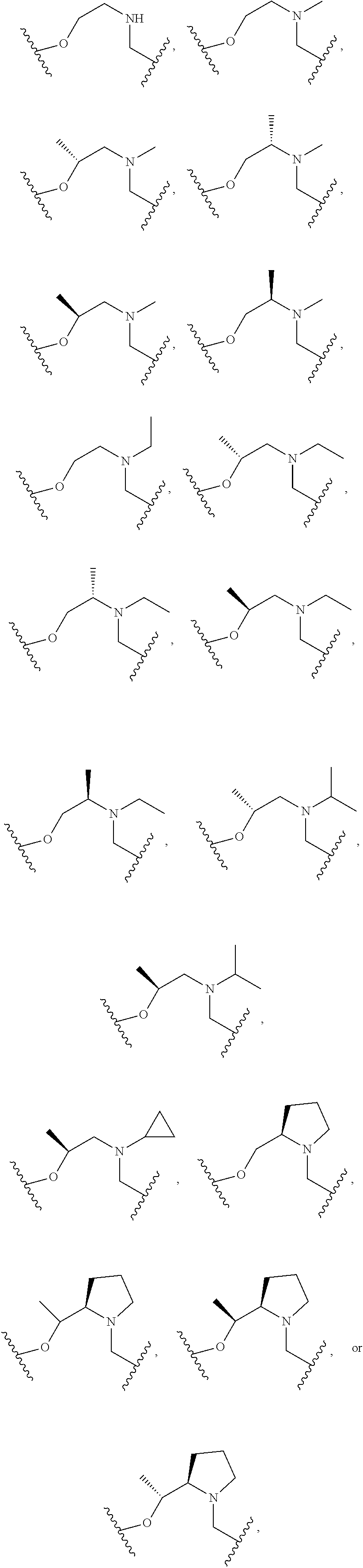

wherein each “ [symbol] ” represents a point of covalent attachment, and each hydrogen is independently optionally substituted with deuterium.

21. The compound of any one of the preceding embodiments, or a pharmaceutically acceptable salt thereof, wherein R⁸ is H or C₁-C₆ alkyl.

22. The compound of any one of the preceding embodiments, or a pharmaceutically acceptable salt thereof, wherein R⁹ is H, deuterium, halogen, C₁-C₆ alkyl, wherein each hydrogen atom in C₁-C₆ alkyl is independently optionally substituted with deuterium.

23. The compound of any one of the preceding embodiments, or a pharmaceutically acceptable salt thereof, wherein R⁹ is H, deuterium, halogen, —CH₃, or —CD₃.

24. The compound of embodiment 1, selected from the group consisting of

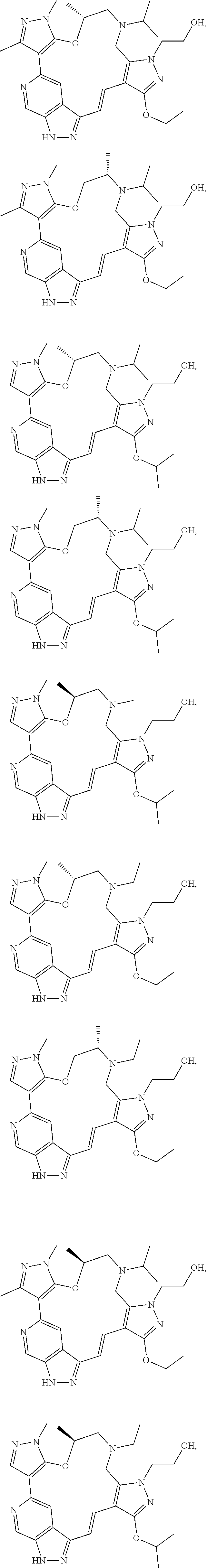

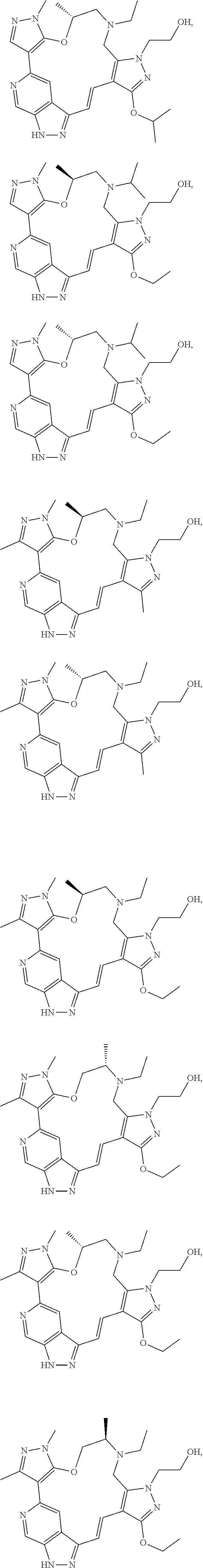

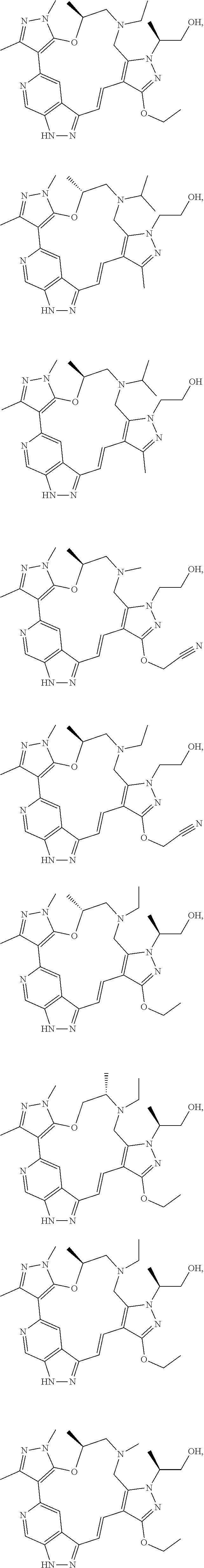

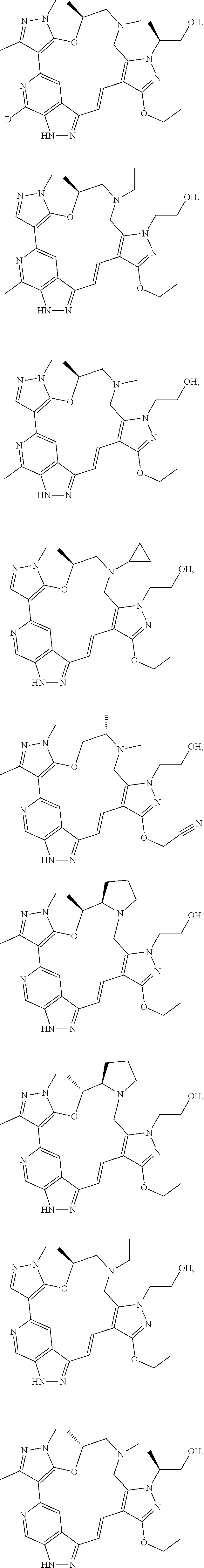

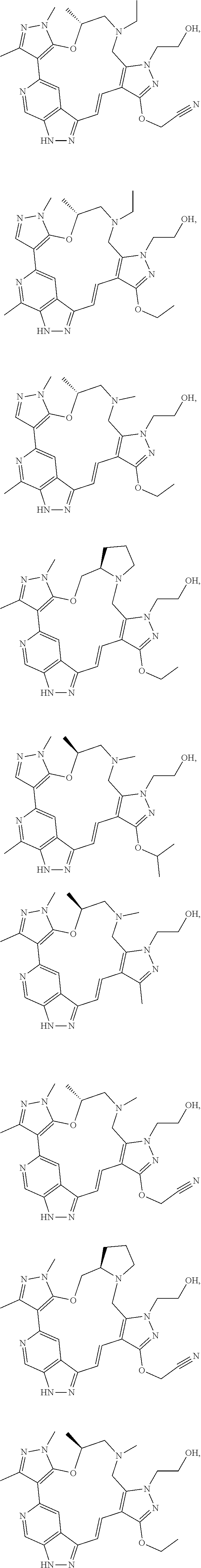

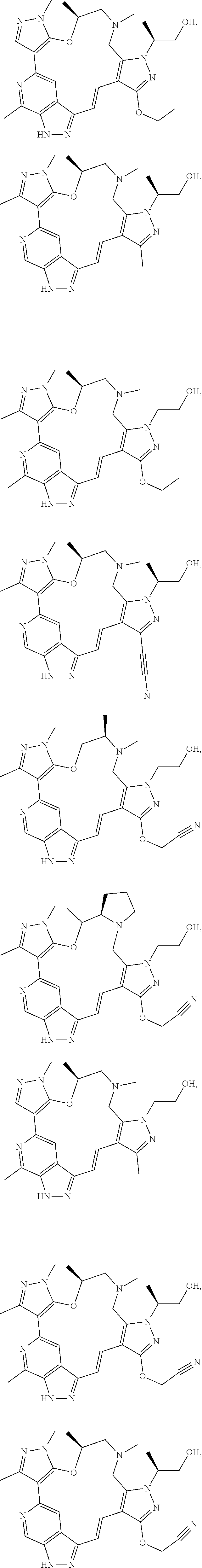

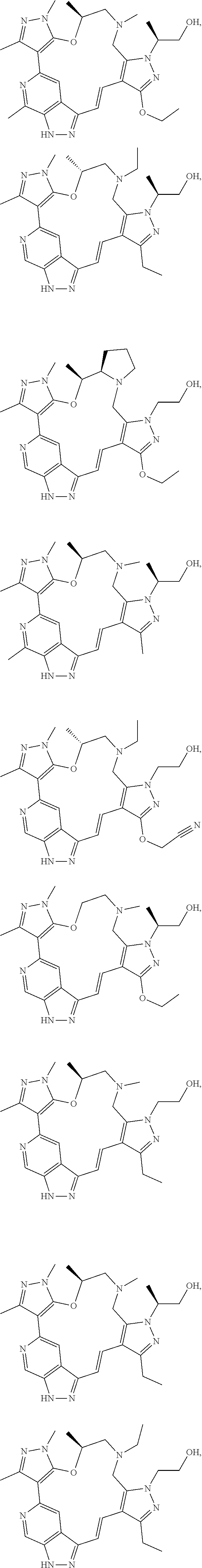

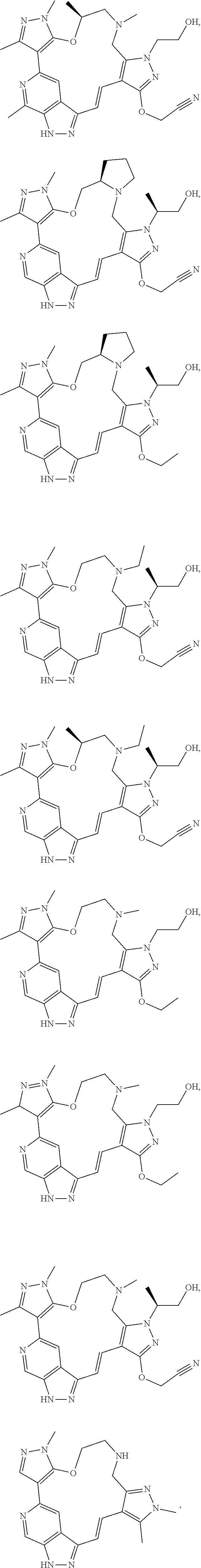

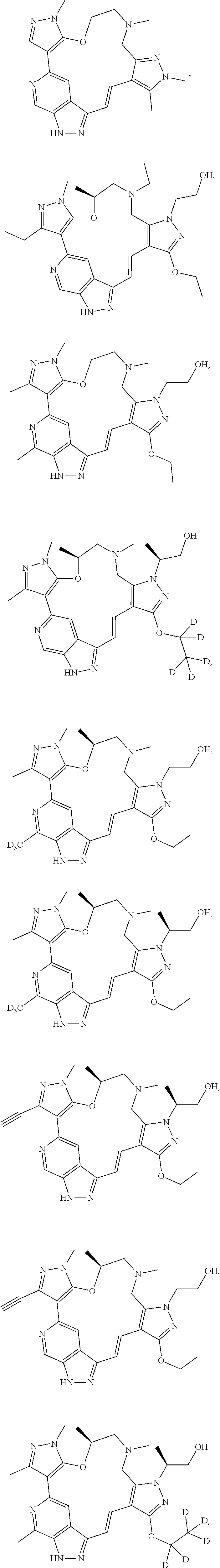

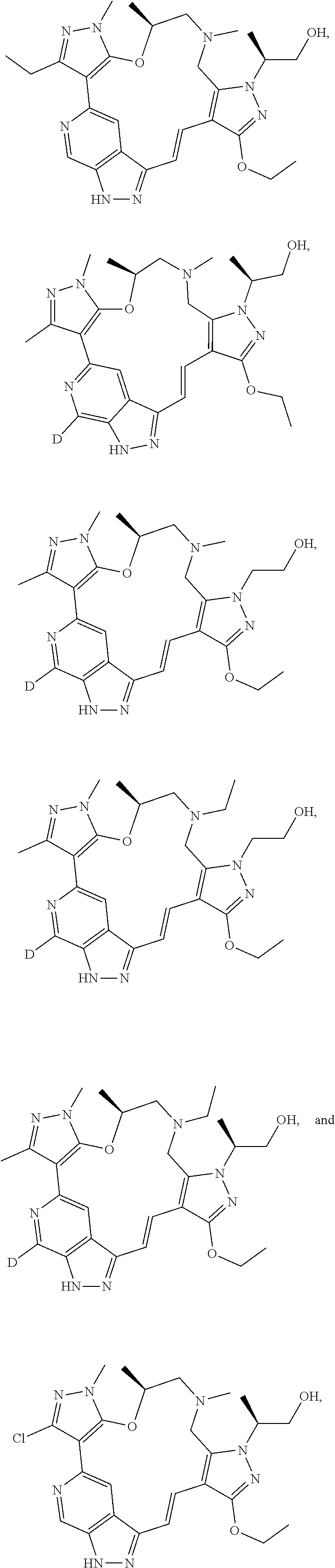

or a pharmaceutically acceptable salt thereof.

25. A pharmaceutical composition comprising a compound of any one of the preceding embodiments, and optionally one or more excipients.

26. A method of treating disease in a subject comprising, administering a therapeutically effective amount of a compound of any one of embodiments 1 to 24, or a pharmaceutical composition of embodiment 25.

27. A compound according to any one of embodiments 1 to 24, for use in a method of treating disease in a subject.

28. Use of a compound according to any one of embodiments 1 to 24, in the manufacture of a medicament for the treatment of disease in a subject.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 g/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, acute myeloid leukemia, congenital mesoblastic nephroma, congenital fibrosarcomas, Ph-like acute lymphoblastic leukemia, thyroid carcinoma, skin cutaneous melanoma, head and neck squamous cell carcinoma, pediatric glioma CML, prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes, lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma. Pain includes, for example, pain from any source or etiology, including cancer pain, pain from chemotherapeutic treatment, nerve pain, pain from injury, or other sources. Autoimmune diseases include, for example, rheumatoid arthritis, Sjogren syndrome, Type I diabetes, and lupus. Exemplary neurological diseases include Alzheimer's Disease, Parkinson's Disease, Amyotrophic lateral sclerosis, and Huntington's disease. Exemplary inflammatory diseases include atherosclerosis, allergy, and inflammation from infection or injury.

In one aspect, the compounds and pharmaceutical compositions of the disclosure specifically target tyrosine receptor kinases, in particular EGFR, including EGFR having one or more mutations, such as L858R, Δ746-750, Δ746-750/C979S, L858R/T790M, L858R/T790M/C979S, and D770_N771insNPG. Thus, these compounds and pharmaceutical compositions can be used to prevent, reverse, slow, or inhibit the activity of one or more of these kinases. In preferred embodiments, methods of treating a target cancer are described. In other embodiments, methods are for treating lung cancer, such as non-small cell lung cancer.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to upregulation of EGFR, including a cancer cell having one or more EGFR mutations, such as L858R, Δ746-750, Δ746-750/C979S, L858R/T790M, L858R/T790M/C979S, and D770_N771insNPG, while maintaining good selectivity over wild-type EGFR.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g., crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids. For pain indications, suitable combination agents include anti-inflammatories such as NSAIDs. The pharmaceutical compositions of the disclosure may additional comprise one or more of such active agents, and methods of treatment may additionally comprise administering an effective amount of one or more of such active agents.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)-(IV).

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| °C | degrees Celsius |
| PE | petroleum ether |
| EA or EtOAc | ethyl acetate |
| R_{f} | retardation factor |
| N | normal |
| J | coupling constant |
| RT | Room Temperature |
| mM | millimolar |
| μM or uM | micromolar |
| nM | nanomolar |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| DMSO-d₆ | deuterated dimethyl sulfoxide |
| DMSO | dimethyl sulfoxide |
| TMSCl | trimethylsilyl chloride |
| Boc | tert-butyloxycarbonyl |
| TFA | trifluoroacetic acid |
| TFAA | trifluoroacetic anhydride |
| DMAP | 4-(dimethylamino)pyridine |
| ATP | adenosine triphosphate |
| IC₅₀ | half maximal inhibitory concentration |
| U/mL | units of activity per milliliter |
| Hex | hexanes |
| MeCN or ACN | Acetonitrile |
| DIPEA or DIEA or | N,N-diisopropylethylamine |
| Hunig's Base | |
| TEA | triethylamine |
| KHMDS | potassium bis(trimethylsilyl)amide |
| DIAD | diisopropyl azodicarboxylate |
| DBAD | di-tert-butyl azodicarboxylate |
| MeTHF or 2-MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DPPF | 1,1′-Bis(diphenylphosphino)ferrocene |
| DBU | 1,8-diazabicyclo[5.4.0]undec-7-ene |
| AcOH | Acetic Acid |
| FA | formic acid |
| TBS | Tert-Butyldimethylsilyl |
| TMS | Trimethylsilyl |
| SEM | [2-(Trimethylsilyl)ethoxy]methyl |
| Pd(dppf)Cl₂ | [1,1′-Bis(diphenylphosphino)ferrocene]dichloro- |
| | palladium(II) |
| Pd(dtbpf)Cl₂ | [1,1′-Bis(di-tert- |
| | butylphosphino)ferrocene]dichloropalladium(II) |
| TPPO | Triphenylphosphine oxide |
| Pd₂(dba)₃ | Tris(dibenzylideneacetone)dipalladium(0) |
| PPh₃ | Triphenyl phosphine |
| Tos or Ts | toluenesulfonyl |
| p-TSA | Para-Tolylsulfonic acid |
| n-BuOH | n-butanol |
| t-BuOH | Tert-Butanol |
| Pd(amphos)Cl₂ | Dichlorobis[di-tert-butyl(4- |
| | dimethylaminophenyl)phosphine]palladium(II) |
| mCPBA | Meta-Chloroperoxy benzoic acid |
| BPD or B₂Pin₂ or | 4,4,5,5-tetramethyl-2-(4,4,5,5-tetramethyl-1,3,2- |
| Bis(pinacolato)diboron | dioxaborolan-2-yl)-1,3,2-dioxaborolane |
| MTBE | Methy tert-Butyl Ether |
| TBAC | Tetrabutylammonium chloride |
| TBAF | Tert butyl ammonium fluoride |
| LAH | Lithium aluminium hydride |
| HATU | Hexafluorophosphate Azabenzotriazole |
| | Tetramethyl Uronium |
| DMA or DMAc | Dimethylacetamide |
| PPTS | Pyridinium p-Toluenesulfonate |
| DHP | 3,4-Dihydropyran |
| THP | Tetrahydropyran |
| NMP | N-Methyl-2-pyrrolidone |
| DIBAL-H | Diisobutylaluminium hydride |
| OTf | triflate |
| tert-BuBrettPhos-Pd- | [(2-Di-tert-butylphosphino-3,6-dimethoxy-2′,4′,6′- |
| G3 | triisopropyl-1,1′-biphenyl)-2-(2′-amino-1,1′- |
| | biphenyl)]palladium(II) methanesulfonate |
| Cy3P Pd G3 | [(Tricyclohexylphosphine)-2-(2′-amino- |
| | biphenyl)]palladium(II) methanesulfonate |
| Xphos | dicyclohexyl-[2-(2,4,6-triisopropylphenyl) |
| | phenyl]phosphane |
| Pd-PEPPSI | [1,3-Bis(2,6-Di-3-pentylphenyl)imidazol-2- |
| | ylidene](3-chloropyridyl)palladium(II) dichloride |
| NBS | N-Bromosuccinimide |
| NIS | N-Iodosuccinimide |

The proposed targets can be prepared via the conventional chemistry or following the general schemes as shown below.

The following starting materials and intermediates can be obtained from commercial sources or using known literature procedures: (2S)-2-methyloxirane, (2R)-2-methyloxirane, propan-2-amine, iodoethane, 2-bromoethoxy-tert-butyl-dimethyl-silane, (2R)-1-(isopropylamino) propan-2-ol, 5-bromo-1H-pyrazolo[3,4-c]pyridine, 2,5-dimethylpyrazol-3-ol, 2,4-dimethylpyridin-3-amine, 2,6-dimethylpyridine, 2-methylpyrazol-3-ol, methyl 3-methyl-1H-pyrazole-5-carboxylate (a.k.a. methyl 5-methyl-1H-pyrazole-3-carboxylate), 2-[tert-butyl(dimethyl)silyl]oxyethanol, (2R)-1-(ethylamino)propan-2-ol, (2S)-2-aminopropan-1-ol, (2R)-2-aminopropan-1-ol, (2R)-propane-1,2-diol, (2S)-propane-1,2-diol, (2R)-1-(isopropylamino)propan-2-ol, (2S)-1-(isopropylamino)propan-2-ol, (2R)-1-tert-butoxycarbonylpyrrolidine-2-carboxylic acid, N-methoxymethanamine, 2-(methylamino)ethanol, ethanamine, 2-iodoacetonitrile, tert-butyl N-[(2R)-2-hydroxypropyl]carbamate, tert-butyl N-[(1R)-2-hydroxy-1-methyl-ethyl]carbamate, tert-butyl N-[(]S)-2-hydroxy-1-methyl-ethyl]carbamate, tert-butyl N-[(2S)-2-hydroxypropyl]carbamate, methyl 3-bromo-1H-pyrazole-5-carboxylate, methyl 3-hydroxy-1H-pyrazole-5-carboxylate, ethyl 3-hydroxy-1H-pyrazole-5-carboxylate, tert-butyl N-(2-bromoethyl)carbamate, (2,2,2-trifluoroacetyl) 2,2,2-trifluoroacetate, N-ethyl-2,2,2-trifluoro-N-[(2S)-2-hydroxypropyl]acetamide, [(2R)-pyrrolidin-2-yl]methanol, ethyl 2,4-dioxohexanoate, (R)-1-(ethylamino)propan-2-ol, 1,3-dimethylpyrazol-4-ol, [(1R)-2-(tert-butoxycarbonylamino)-1-methyl-ethyl]methanesulfonate, ethynyl(triisopropyl)silane, cyclopropanamine, ethyl 2,4-dioxopentanoate, 5-(bromomethyl)-4-iodo-1,3-dimethyl-pyrazole, (2S)-1-(cyclopropylamino)propan-2-ol, methyl 1H-pyrazole-5-carboxylate, ethyl (2S)-2-hydroxypropanoate, ethyl 3-ethoxy-1H-pyrazole-5-carboxylate, (2-hydroxyphenyl)boronic acid, 1-methylpyrazol-4-ol, tert-butyl N-(2-hydroxyethyl)-N-methyl-carbamate, 2-isopropoxy-4,4,5,5-tetramethyl-1,3,2-dioxaborolane, ethyl 5-methyl-1H-pyrazole-3-carboxylate, methyl 5-hydroxy-2-methyl-pyrazole-3-carboxylate, 1-iodoethane-1,1,2,2,2-d5, 2-chloro-4-methyl-pyridin-3-amine, 2,2,2-trifluoro-N-[(2R)-2-hydroxypropyl]-N-methyl-acetamide, ethyl 2,5-dimethylpyrazole-3-carboxylate, ethyl 3,3-diethoxyprop-2-enoate, 2,2,2-trifluoro-N-[(2R)-2-hydroxypropyl]-N-methyl-acetamide, methyl 4-methoxy-3-oxo-butanoate, ethyl 1-hydroxycyclopropanecarboxylate, 4,4,4′,4′,5,5,5′,5′-octamethyl-2,2′-bi-1,3,2-dioxaborolane, 1-methyl-1H-pyrazol-5-ol, tert-butyl (2-bromoethyl)carbamate, tert-butyl (R)-(1-oxopropan-2-yl)carbamate, 1,3-dimethyl-1H-pyrazol-5-ol, tert-butyl (R)-(2-hydroxypropyl)carbamate, propanoyl chloride, cyclopropanecarbonyl chloride, cyclopropanol, ethyl 4-chloro-3-oxo-butanoate, 3-bromo-1,1-difluoro-propan-2-ol, and 3,6-dioxabicyclo[3.1.0]hexane; benzyl(triethyl)ammonium chloride, and methyl 3-hydroxy-1-methyl-1H-pyrazole-5-carboxylate.

General Method A: using Ex. 1 as example.

### Preparation of 2-[(10R,17E)-16-ethoxy-6,8,10-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 1)

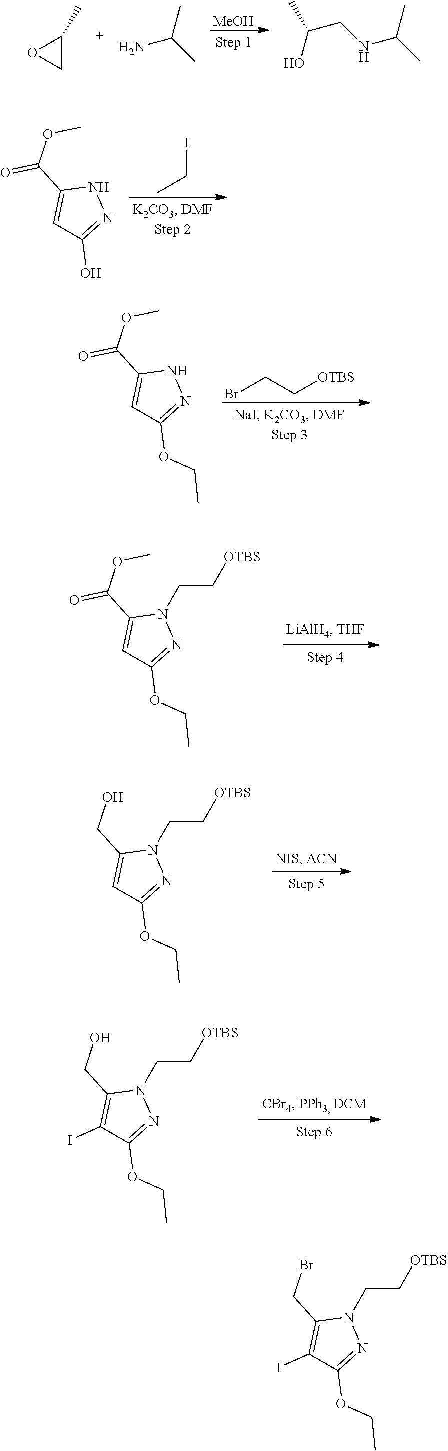

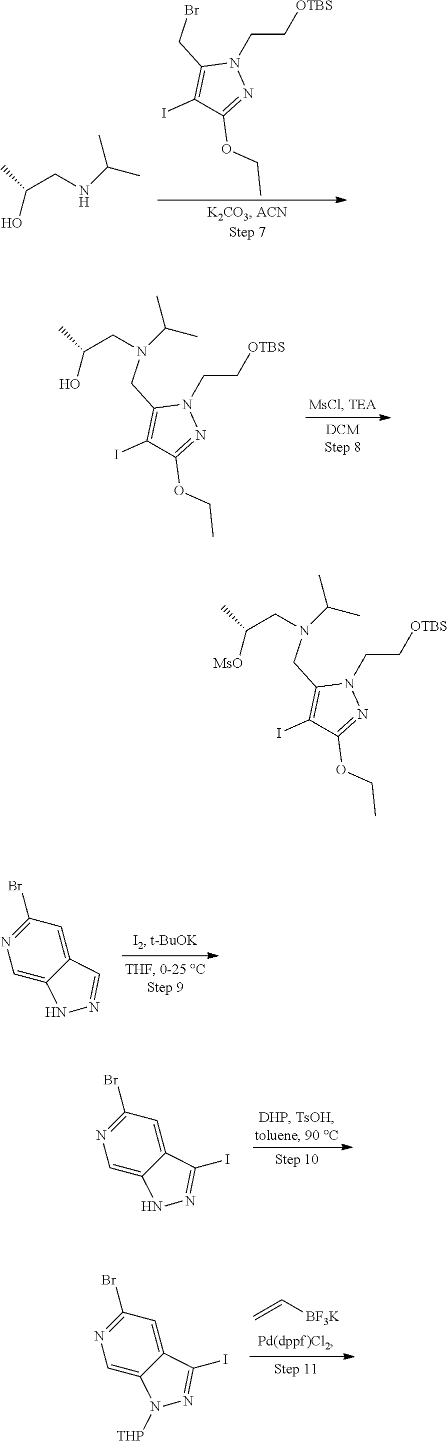

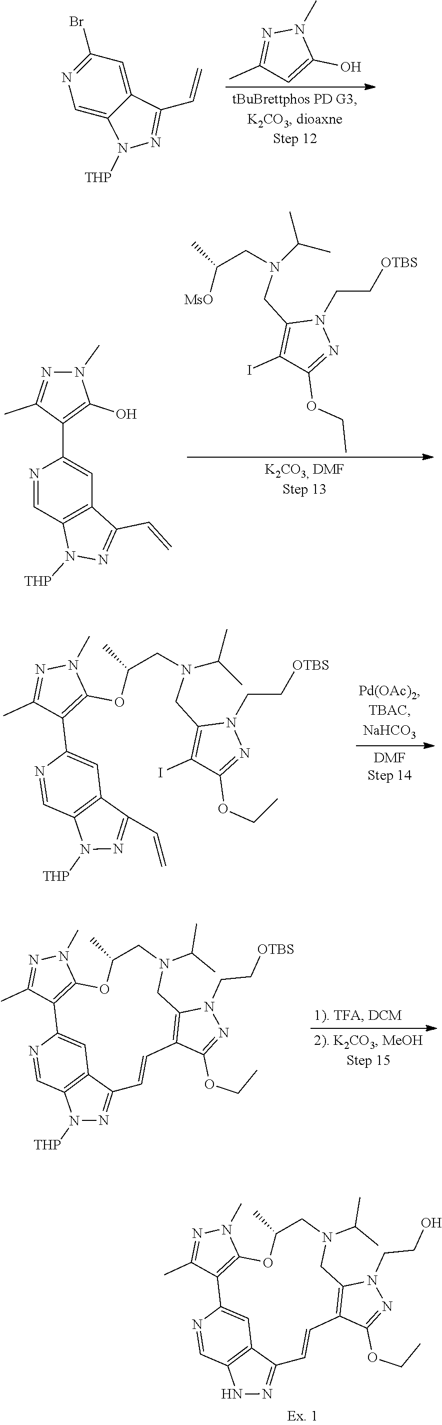

Step 1. A solution of commercially available (2R)-2-methyloxirane (5.00 g, 86.0 mmol, 1 eq) and commercially available propan-2-amine (15.2 g, 258 mmol, 3 eq) in MeOH (80 mL) was stirred at 25° C. for 14 hours. On completion, the mixture was concentrated to give (2R)-1-(isopropylamino) propan-2-ol (8.60 g, 73.3 mmol, 85% yield) as a yellow oil. ¹H NMR (400 MHz, CDCl₃) δ=3.76-3.66 (m, 1H), 2.85-2.69 (m, 2H), 2.40-2.27 (m, 1H), 2.19-1.94 (m, 1H), 1.15 (d, J=6.4 Hz, 3H), 1.09-1.04 (m, 6H).

Step 2. To a solution of commercially available methyl 3-hydroxy-1H-pyrazole-5-carboxylate (15 g, 105.55 mmol, 1 eq), iodoethane (16.46 g, 105.55 mmol, 8.44 mL, 1 eq) in DMF (150 mL) was added K₂CO₃ (43.76 g, 316.65 mmol, 3 eq). The mixture was stirred at 80° C. for 2 hr. On completion, the mixture was diluted with water (200 mL) and extracted with ethyl acetate (50 mL×3). The combined organic phase was washed with water (200 mL×2), dried over Na₂SO₄, filtered, and the filtrate was concentrated. The residue was purified by column chromatography (SiO₂, Petroleum ether/Ethyl acetate=1:0 to 80:20) to give methyl 3-ethoxy-1H-pyrazole-5-carboxylate (10.4 g, 61.12 mmol, 58% yield) as a white solid. LCMS: (M+1:171.1)

Step 3. To a solution of methyl 3-ethoxy-1H-pyrazole-5-carboxylate (10 g, 58.77 mmol, 1 eq) and commercially available 2-bromoethoxy-tert-butyl-dimethyl-silane (21.09 g, 88.15 mmol, 1.5 eq) in DMF (100 mL) was added NaI (8.81 g, 58.77 mmol, 1 eq), K₂CO₃ (24.37 g, 176.30 mmol, 3 eq), and the mixture was stirred at 60° C. for 16 hr. On completion, the mixture was diluted with water (100 mL) and extracted with ethyl acetate (40 mL×3). The combined organic phase was washed with water (80 mL×2), dried over Na₂SO₄, filtered, and the filtrate was concentrated to give a residue. The residue was purified by column chromatography (SiO₂, Petroleum ether/Ethyl acetate=1:0 to 17:83) to give methyl 1-(2-((tert-butyldimethylsilyl)oxy)ethyl)-3-ethoxy-1H-pyrazole-5-carboxylate (12.7 g, 38.66 mmol, 66% yield) as a yellow liquid. LCMS: (M+1:329.5).

Step 4. To a solution of methyl 2-[2-[tert-butyl(dimethyl)silyl]oxyethyl]-5-ethoxy-pyrazole-3-carboxylate (12 g, 36.53 mmol, 1 eq) in THE (120 mL) was added LiAlH₄ (1.39 g, 36.53 mmol, 1 eq) at 0° C. The mixture was stirred at 0° C. for 2 hr. On completion, the mixture was quenched with MeOH (150 mL) at 0° C., and the mixture was concentrated to give a residue. The residue was purified by column chromatography (SiO₂, Petroleum ether/Ethyl acetate=1:0 to 3:1) to give (1-(2-((tert-butyldimethylsilyl)oxy)ethyl)-3-ethoxy-1H-pyrazol-5-yl)methanol (12.7 g, 38.66 mmol, 66% yield) as a yellow liquid. LCMS: (M+1:301.1)

Step 5. To a solution of [2-[2-[tert-butyl(dimethyl)silyl]oxyethyl]-5-ethoxy-pyrazol-3-yl]methanol (7 g, 23.30 mmol, 1 eq) in ACN (70 mL) was added NIS (4.72 g, 20.97 mmol, 0.9 eq) at 0° C., and the mixture was stirred at 25° C. for 2 hr. On completion, the mixture was quenched with sat. Na₂SO₃ (200 mL) at 0° C. and extracted with ethyl acetate (50 mL×3). The combined organic phase was dried over anhydrous Na₂SO₄, filtered, and the filtrate was concentrated to give a residue. The residue was purified by column chromatography (SiO₂, Petroleum ether/Ethyl acetate=1:0 to 80:20) to give (1-(2-((tert-butyldimethylsilyl)oxy)ethyl)-3-ethoxy-4-iodo-1H-pyrazol-5-yl)methanol (7.7 g, 18.06 mmol, 66% yield) as a yellow liquid. ¹H NMR (400 MHz, MeOD) δ=4.60 (d, J=2.0 Hz, 2H), 4.30-4.25 (m, 2H), 4.23 (dt, J=2.6, 4.8 Hz, 2H), 3.95-3.90 (m, 2H), 3.33 (br s, 1H), 1.45-1.39 (m, 3H), 0.84 (d, J=2.0 Hz, 9H), 0.02-0.01 (m, 6H). LCMS: (M+1:427.0).

Step 6. To a solution of [2-[2-[tert-butyl(dimethyl)silyl]oxyethyl]-5-ethoxy-4-iodo-pyrazol-3-yl]methanol (3.5 g, 8.21 mmol, 1 eq) and PPh₃ (2.58 g, 9.85 mmol, 1.2 eq) in DCM (35 mL) was added CBr₄ (3.27 g, 9.85 mmol, 1.2 eq) at 0° C. The mixture was stirred at 25° C. for 1 hr. On completion, the mixture was diluted with water (50 mL) and extracted with DCM (30 mL×3). The combined organic phase was dried over Na₂SO₄, filtered, and the filtrate was concentrated to give a residue. The residue was purified by column chromatography (SiO₂, Petroleum ether/Ethyl acetate=1:0 to 80:20) to give 5-(bromomethyl)-1-(2-((tert-butyldimethylsilyl)oxy)ethyl)-3-ethoxy-4-iodo-1H-pyrazole (2.9 g, 5.93 mmol, 72% yield) (a.k.a. 2-[5-(bromomethyl)-3-ethoxy-4-iodo-pyrazol-1-yl]ethoxy-tert-butyl-dimethyl-silane) as a yellow oil. LCMS: (M+1:490.8).

Step 7. To a solution of (2R)-1-(isopropylamino) propan-2-ol (4.00 g, 34.1 mmol, 1.5 eq) in ACN (260 mL) was added K₂CO₃ (6.29 g, 45.5 mmol, 2 eq) and 5-(bromomethyl)-1-(2-((tert-butyldimethylsilyl)oxy)ethyl)-3-ethoxy-4-iodo-1H-pyrazole (11.1 g, 22.8 mmol, 1 eq). The mixture was stirred at 50° C. for 12 h. On completion, the mixture was filtered and concentrated to give a residue. The residue was purified by column chromatography (SiO₂, PE/THE=1:0 to 5:1) to give (2R)-1-[[2-[2-[tert-butyl(dimethyl)silyl]oxyethyl]-5-ethoxy-4-iodo-pyrazol-3-yl]methyl-isopropyl-amino]propan-2-ol (11.9 g, 22.6 mmol, 99% yield) as a colorless oil. LCMS: (M+1:526.5).

Step 8. To a solution of (2R)-1-[[2-[2-[tert-butyl(dimethyl)silyl]oxyethyl]-5-ethoxy-4-iodo-pyrazol-3-yl]methyl-isopropyl-amino]propan-2-ol (11.9 g, 22.6 mmol, 1 eq) in DCM (120 mL) was added TEA (11.5 g, 113 mmol, 5 eq) and MsCl (7.82 g, 68.3 mmol, 3.02 eq). The mixture was stirred at 0° C. for 30 min. On completion, the mixture was quenched with dilute citric acid (120 mL) and extracted with DCM (60 mL×3). The combined organic phase was dried over anhydrous sodium sulfate, filtered and concentrated to give [(1R)-2-[[2-[2-[tert-butyl(dimethyl)silyl]oxyethyl]-5-ethoxy-4-iodo-pyrazol-3-yl]methyl-isopropyl-amino]-1-methyl-ethyl]methanesulfonate (12.7 g, 21.1 mmol, 93% yield) as a white oil.

Step 9. To a solution of commercially available 5-bromo-1H-pyrazolo[3,4-c]pyridine (23.0 g, 116 mmol, 1 eq), t-BuOK (26.0 g, 232 mmol, 2 eq) in THE (300 mL) was added a solution of I₂ (32.4 g, 127 mmol, 1.1 eq) in THE (100 mL) dropwise at 0° C. The mixture was stirred at 0° C. for 3 hrs. On completion, the mixture was quenched with sat. NaHSO₃ (100 mL), diluted with H₂O (300 mL), and extracted with ethyl acetate (3×300 mL). The organic layers were washed with brine (3×100 mL), dried over anhydrous Na₂SO₄, filtered, and concentrated in vacuo to give 5-bromo-3-iodo-1H-pyrazolo[3,4-c]pyridine (37.5 g, 115 mmol, 99.67% yield) as a yellow solid. ¹H NMR (400 MHz, DMSO-d₆) δ=8.80 (s, 1H), 7.55 (s, 1H). LCMS: (M+1: 323.6).

Step 10. To a solution of 5-bromo-3-iodo-1H-pyrazolo[3,4-c]pyridine (25.0 g, 77.1 mmol, 1 eq) in toluene (250 mL) was added TsOH (2.66 g, 15.4 mmol, 0.2 eq) and 3,4-dihydro-2H-pyran (DHP) (16.2 g, 192 mmol, 2.5 eq). The mixture was stirred at 90° C. for 2 hrs. On completion, the mixture was washed with NH₄Cl solution (2×100 mL), washed with brine (2×100 mL), dried with anhydrous Na₂SO₄, filtered, and concentrated in vacuo. The residue was purified by column chromatography (SiO₂, PE/EA=100/8) to give 5-bromo-3-iodo-1-tetrahydropyran-2-yl-pyrazolo[3,4-c]pyridine (22.3 g, 54.6 mmol, 70.81% yield) as a yellow solid. ¹H NMR (400 MHz, DMSO-d₆) δ=9.08 (s, 1H), 7.72 (s, 1H), 6.00 (dd, J=1.6, 8.8 Hz, 1H), 3.92-3.84 (m, 1H), 3.80-3.72 (m, 1H), 2.35-2.26 (m, 1H), 2.06-1.96 (m, 2H), 1.79-1.66 (m, 1H), 1.63-1.55 (m, 2H). LCMS: (M+1: 409.8).

Step 11. To a solution of 5-bromo-3-iodo-1-tetrahydropyran-2-yl-pyrazolo[3,4-c]pyridine (12.0 g, 29.4 mmol, 1 eq), potassium vinyltrifluoroborate(19.7 g, 147 mmol, 5 eq) in a mixed solvent system of dioxane (120 mL) and H₂O (24 mL) was added Pd(dppf)Cl₂ (2.15 g, 2.94 mmol, 0.1 eq) and Na₂CO₃ (9.35 g, 88.2 mmol, 3 eq). The mixture was stirred at 40° C. for 72 hrs under N₂. On completion, the mixture was filtered and concentrated to give a residue. The residue was purified by column chromatography (SiO₂, PE/EA=50/1) to give 5-bromo-1-tetrahydropyran-2-yl-3-vinyl-pyrazolo[3,4-c]pyridine (8.6 g, 27.9 mmol, 94.89% yield) as a yellow solid. ¹H NMR (400 MHz, DMSO-d₆) δ=9.06 (s, 1H), 8.30 (s, 1H), 7.01 (dd, J=11.6, 18.0 Hz, 1H), 6.21 (d, J=18.0 Hz, 1H), 6.03-5.93 (m, 1H), 5.59 (d, J=11.6 Hz, 1H), 3.93-3.84 (m, 1H), 3.81-3.72 (m, 1H), 2.39-2.27 (m, 1H), 2.05-1.97 (m, 2H), 1.78-1.69 (m, 1H), 1.63-1.55 (m, 2H). LCMS: (M+1:309.8).

Step 12. To a solution of 5-bromo-1-tetrahydropyran-2-yl-3-vinyl-pyrazolo[3,4-c]pyridine (7.00 g, 22.7 mmol, 1 eq.) in dioxane (210 mL) was added K₂CO₃ (6.28 g, 45.4 mmol, 2 eq), commercially available 2,5-dimethylpyrazol-3-ol (3.06 g, 27.3 mmol, 1.2 eq) and [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (1.36 g, 1.59 mmol, 0.07 eq) under N₂. The mixture was stirred at 130° C. for 12 h. On completion, the mixture was filtered and concentrated to give a residue. The residue was purified by column chromatography (SiO₂, DCM/MeOH=1:0 to 15:1) to give 2,5-dimethyl-4-(1-tetrahydropyran-2-yl-3-vinyl-pyrazolo[3,4-c]pyridin-5-yl)pyrazol-3-ol (5.10 g, 15.0 mmol, 66% yield) as a brown solid. LCMS: (M+1:340.1).

Step 13. To a solution of 2,5-dimethyl-4-(1-tetrahydropyran-2-yl-3-vinyl-pyrazolo[3,4-c]pyridin-5-yl)pyrazol-3-ol (5.70 g, 16.8 mmol, 1 eq.) and [(1R)-2-[[2-[2-[tert-butyl(dimethyl)silyl]oxyethyl]-5-ethoxy-4-iodo-pyrazol-3-yl]methyl-isopropyl-amino]-1-methyl-ethyl]methanesulfonate (12.2 g, 20.2 mmol, 1.2 eq) in DMF (330 mL) was added K₂CO₃(6.96 g, 50.4 mmol, 3 eq). The mixture was stirred at 60° C. for 12 h. On completion, the mixture was quenched with water (660 mL) and extracted with ethyl acetate (150 mL×3). The combined organic phase was dried over anhydrous sodium sulfate, filtered, and concentrated to give a residue. The residue was purified by column chromatography (SiO₂, PE:EA=1:0 to 1:2) to give (2S)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-isopropyl-propan-1-amine (8.83 g, 10.4 mmol, 62% yield) as a yellow solid. LCMS: (M+1:847.7).

Step 14. To a solution of (2S)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-isopropyl-propan-1-amine (8.83 g, 10.4 mmol, 1 eq) in DMF (445 mL) was added TBAC (2.90 g, 10.4 mmol, 1 eq), NaHCO₃ (2.19 g, 26.1 mmol, 2.5 eq) and Pd(OAc)₂ (468 mg, 2.09 mmol, 0.2 eq), the mixture was degassed and purged with N₂ for 3 times, and then the mixture was stirred at 140° C. for 1 h under N₂ atmosphere. On completion, the mixture was quenched with water (890 mL) and extracted with ethyl acetate (450 mL×3). The combined organic phase was dried over anhydrous sodium sulfate, filtered, and concentrated to give a residue. The residue was purified by column chromatography (SiO₂, PE:EA=1:0 to 1:1) to give (10R,17E)-14-(2-{[tert-butyl(dimethyl)silyl]oxy}ethyl)-16-ethoxy-6,8,10-trimethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (3.30 g, 4.59 mmol, 44% yield) as a yellow solid. LCMS: (M+1:719.8).

Step 15. To a solution of (10R,17E)-14-(2-{[tert-butyl(dimethyl)silyl]oxy}ethyl)-16-ethoxy-6,8,10-trimethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (3.30 g, 4.59 mmol, 1 eq) in DCM (25 mL) was added TFA (38.4 g, 337 mmol, 73.3 eq). The mixture was stirred at 25° C. for 12 h. On completion, the mixture was concentrated to give a brown solid (2.83 g, crude). This brown solid (2.83 g, 4.59 mmol, 1 eq) was dissolved in MeOH (29 mL), and K₂CO₃ (6.34 g, 45.9 mmol, 10 eq) was added. The mixture was stirred at 25° C. for 0.5 h. On completion, the mixture was concentrated to give a residue. The residue was purified by column chromatography (SiO₂, PE/THF=1:0 to 1:3) to give 2-[(10R,17E)-16-ethoxy-6,8,10-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (1.15 g, 2.21 mmol, 48.13% yield) as a white solid. LCMS: (M+1: 521.3). ¹H NMR for Ex. 1 can be found in the below NMR table. The final structure was confirmed by X-ray crystal structure determination.

### Preparation of 2-[(10S,17E)-8,10,12-trimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 5)

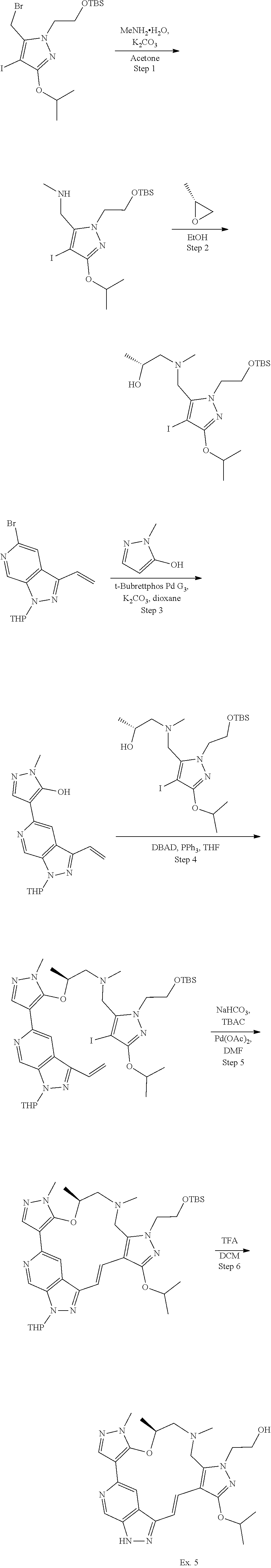

Step 1. To a solution of MeNH₂ (41.1 g, 397 mmol, 30% purity, 20 eq) in acetone (800 mL) was added K₂CO₃ (8.24 g, 59.6 mmol, 3 eq) at 0° C. Then 2-[5-(bromomethyl)-4-iodo-3-isopropoxy-pyrazol-1-yl]ethoxy-tert-butyl-dimethyl-silane, which was prepared according to the method described in Ex. 3/Ex. 4, (10 g, 19.8 mmol, 1 eq) in acetone (200 mL) was added dropwise at 0° C. The mixture was stirred at 0° C. for 12 hr. On completion, the mixture was quenched with water (1000 mL) and extracted with ethyl acetate (1000 mL×3). The combined organic phase was dried over anhydrous sodium sulfate, filtered, and concentrated to give a residue. The residue was purified by column chromatography (SiO₂, DCM/MeOH=20:1 to 15:1) to give 1-[2-[2-[tert-butyl(dimethyl)silyl]oxyethyl]-4-iodo-5-isopropoxy-pyrazol-3-yl]-N-methyl-methanamine (7.00 g, 15.4 mmol, 77.7% yield) as a yellow oil. LCMS: (M+1: 454.1).

Step 2. A solution of commercially available (2R)-2-methyloxirane (1.92 g, 33.0 mmol, 2.32 mL, 3 eq) and 1-[2-[2-[tert-butyl(dimethyl)silyl]oxyethyl]-4-iodo-5-isopropoxy-pyrazol-3-yl]-N-methyl-methanamine (5.00 g, 11.0 mmol, 1 eq) in EtOH (125 mL) was stirred at 80° C. for 12 hr. On completion, the mixture was concentrated to give (2R)-1-[[2-[2-[tert-butyl(dimethyl)silyl]oxyethyl]-4-iodo-5-isopropoxy-pyrazol-3-yl]methyl-methyl-amino]propan-2-ol (5.5 g, 10.7 mmol, 97.5% yield) as a yellow oil. LCMS: (M+1:512.2).

Step 3. To a solution of commercially available 2-methylpyrazol-3-ol (3.06 g, 31.1 mmol, 1.2 eq), 5-bromo-1-tetrahydropyran-2-yl-3-vinyl-pyrazolo[3,4-c]pyridine, which was prepared according to the method described in Ex. 1, (8.00 g, 25.9 mmol, 1 eq) in dioxane (80 mL) was added K₂CO₃ (10.7 g, 77.8 mmol, 3 eq) and [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (1.77 g, 2.08 mmol, 0.08 eq). The mixture was stirred at 100° C. for 2 h. On completion, the mixture was filtered and concentrated to give a residue. The residue was purified by column chromatography (SiO₂, DCM/MeOH=20:1 to 15:1) to give 2-methyl-4-(1-tetrahydropyran-2-yl-3-vinyl-pyrazolo[3,4-c]pyridin-5-yl)pyrazol-3-ol (4.30 g, 13.2 mmol, 51% yield) as a red solid. LCMS: (M+1:326.0).

Step 4. To a solution of 2-methyl-4-(1-tetrahydropyran-2-yl-3-vinyl-pyrazolo[3,4-c]pyridin-5-yl)pyrazol-3-ol (699 mg, 2.15 mmol, 1.1 eq), (2R)-1-[[2-[2-[tert-butyl(dimethyl)silyl]oxyethyl]-4-iodo-5-isopropoxy-pyrazol-3-yl]methyl-methyl-amino]propan-2-ol from step 2 (1.00 g, 1.95 mmol, 1 eq) in THF (15 mL) was added PPh₃ (1.13 g, 4.30 mmol, 2.2 eq) and DBAD (990 mg, 4.30 mmol, 2.2 eq). The mixture was stirred at 25° C. for 2 h. On completion, the mixture was filtered and concentrated to give a residue. The residue was purified by column chromatography (SiO₂, PE/THF=2:1 to 1:1) to give (2S)-N-[[2-[2-[tert-butyl(dimethyl)silyl]oxyethyl]-4-iodo-5-isopropoxy-pyrazol-3-yl]methyl]-N-methyl-2-[2-methyl-4-(1-tetrahydropyran-2-yl-3-vinyl-pyrazolo[3,4-c]pyridin-5-yl)pyrazol-3-yl]oxy-propan-1-amine (1.50 g, 1.83 mmol, 94% yield) as a white solid. LCMS: (M+1:819.1).

Step 5. To a solution of (2S)-N-[[2-[2-[tert-butyl(dimethyl)silyl]oxyethyl]-4-iodo-5-isopropoxy-pyrazol-3-yl]methyl]-N-methyl-2-[2-methyl-4-(1-tetrahydropyran-2-yl-3-vinyl-pyrazolo[3,4-c]pyridin-5-yl)pyrazol-3-yl]oxy-propan-1-amine (1.3 g, 1.59 mmol, 1 eq), TBAC (441 mg, 1.59 mmol, 1 eq) in DMF (130 mL) was added NaHCO₃ (333 mg, 3.97 mmol, 2.5 eq) and Pd(OAc)₂ (71.2 mg, 317 mol, 0.2 eq). The mixture was stirred at 130° C. for 2 h under N₂ atmosphere. On completion, the mixture was quenched with water (200 mL) and extracted with ethyl acetate (250 mL×3). The combined organic phase was dried over anhydrous sodium sulfate, filtered and concentrated to give a residue. The residue was purified by column chromatography (SiO₂, Petroleum ether/THF=1:1 to 1:1) to give (10S,17E)-14-(2-{[tert-butyl(dimethyl)silyl]oxy}ethyl)-8,10,12-trimethyl-2-(oxan-2-yl)-16-[(propan-2-yl)oxy]-2,10,11,12,13,14-hexahydro-8H-5,3-(azenometheno)tripyrazolo[3,4-f:3′,4′-j:4″,3″-n][1,4]oxazacyclopentadecine (250 mg, 362 μmol, 23% yield) as a yellow solid. LCMS: (M+1: 691.2).

Step 6. To a solution of (10S,17E)-14-(2-{[tert-butyl(dimethyl)silyl]oxy}ethyl)-8,10,12-trimethyl-2-(oxan-2-yl)-16-[(propan-2-yl)oxy]-2,10,11,12,13,14-hexahydro-8H-5,3-(azenometheno)tripyrazolo[3,4-f:3′,4′-j:4″,3″-n][1,4]oxazacyclopentadecine (200 mg, 289 μmol, 1 eq) in DCM (5.00 mL) was added TFA (153 g, 1.35 mol, 4650 eq). The mixture was stirred at 25° C. for 2h. On completion, the mixture was concentrated to give Ex. 5 (58.7 mg, 119 mol, 41% yield) as a yellow solid. LCMS: (M+1:493.1). ¹H NMR for Ex. 5 can be found in the below NMR table.

Preparation of 2-[(10R,17E)-16-ethoxy-12-ethyl-8,10-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 6) and 2-[(11S,17E)-16-ethoxy-12-ethyl-8,11-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 7).

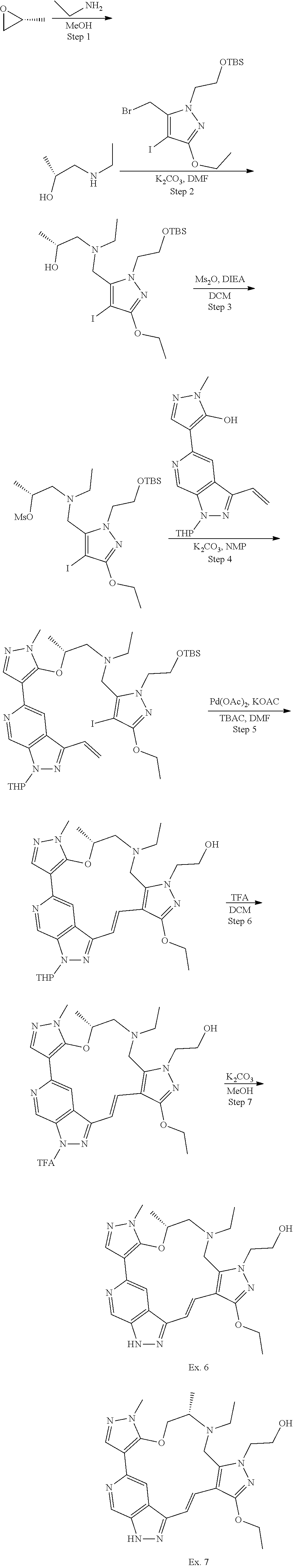

Step 1. To a solution of commercially available (2R)-2-methyloxirane (5 g, 86.1 mmol, 1 eq) in MeOH (50 mL) was added commercially available ethanamine (2 M, 129 mL, 3 eq). The mixture was stirred at 25° C. for 12 h. On completion, the mixture was concentrated to give (2R)-1-(ethylamino) propan-2-ol (7.55 g, 73.2 mmol, 85% yield) as a brown solid. ¹H NMR (400 MHz, CDCl₃) δ=3.85-3.68 (m, 1H), 2.73-2.56 (m, 3H), 2.44-2.34 (m, 2H), 1.18-1.05 (m, 6H).

Step 2. To a solution of 5-(bromomethyl)-1-(2-((tert-butyldimethylsilyl)oxy)ethyl)-3-ethoxy-4-iodo-1H-pyrazole, which was prepared according to the method described in Ex. 1, (1.00 g, 2.04 mmol, 1.00 eq.) and (2R)-1-(ethylamino) propan-2-ol (316 mg, 3.07 mmol, 1.50 eq.) in DMF (10 mL) was added K₂CO₃ (847 mg, 6.13 mmol, 3.00 eq). The mixture was stirred at 80° C. for 2 hr. On completion, the reaction mixture was diluted with H₂O (15 mL) and extracted with EA (20 mL×3). The combined organic layers were washed with H₂O (8 mL), dried over Na₂SO₄, filtered, and concentrated under reduced pressure to give a residue. The residue was purified by column chromatography (SiO₂, Petroleum ether/Ethyl acetate=1:0 to 4:1) to give (2R)-1-[[2-[2-[tert-butyl (dimethyl) silyl]oxyethyl]-5-ethoxy-4-iodo-pyrazol-3-yl]methyl-ethyl-amino]propan-2-ol (1.04 g, 1.95 mmol, 96% yield, 96% purity) as a white liquid. ¹H NMR (400 MHz, DMSO-d₆) δ=4.28 (d, J=4.4 Hz, 1H), 4.23-4.18 (m, 2H), 4.16-4.10 (m, 2H), 3.81 (t, J=5.2 Hz, 2H), 3.62 (d, J=13.6 Hz, 2H), 3.53-3.45 (m, 1H), 3.32 (s, 3H), 2.88 (s, 1H), 2.73 (s, 1H), 2.35-2.27 (m, 1H), 2.23 (d, J=6.4, 11.6 Hz, 1H), 1.28 (t, J=7.2 Hz, 3H), 0.97 (s, 3H), 0.77 (s, 9H), −0.09-0.12 (m, 6H).

Step 3. To a solution of (2R)-1-[[2-[2-[tert-butyl(dimethyl)silyl]oxyethyl]-5-ethoxy-4-iodo-pyrazol-3-yl]methyl-ethyl-amino]propan-2-ol (1.00 g, 1.95 mmol, 1.00 eq) and DIEA (758 mg, 5.86 mmol, 3.00 eq) in DCM (10 mL) was added methylsulfonyl methanesulfonate (511 mg, 2.93 mmol, 1.50 eq) at 0° C. The mixture was stirred at 25° C. for 1 h. On completion, the reaction mixture was diluted with H₂O (15 mL) and extracted with DCM (12 mL×3). The combined organic layers were washed with H₂O (8 mL), dried over Na₂SO₄, filtered, and concentrated under reduced pressure to give [(1R)-2-[[2-[2-[tert-butyl (dimethyl) silyl]oxyethyl]-5-ethoxy-4-iodo-pyrazol-3-yl]methyl-ethyl-amino]-1-methyl-ethyl]methanesulfonate (1.10 g, crude) as an orange oil. LCMS: (M+1: 590.3).

Step 4. To a solution of [(1R)-2-[[2-[2-[tert-butyl(dimethyl)silyl]oxyethyl]-5-ethoxy-4-iodo-pyrazol-3-yl]methyl-ethyl-amino]-1-methyl-ethyl]methanesulfonate (1.00 g, 1.70 mmol, 1.00 eq) in NMP (20 mL) was added 2-methyl-4-(1-tetrahydropyran-2-yl-3-vinyl-pyrazolo[3,4-c]pyridin-5-yl)pyrazol-3-ol, which was prepared according to the methods described in Ex. 5, (552 mg, 1.70 mmol, 1.00 eq) and K₂CO₃ (703 mg, 5.09 mmol, 3.00 eq). The mixture was stirred at 80° C. for 2 hr. On completion, the reaction mixture was diluted with H₂O (150 mL) and extracted with EA (120 mL×3). The combined organic layers were washed with H₂O (80 mL), dried over Na₂SO₄, filtered and concentrated under reduced pressure to give a residue. The residue was purified by column chromatography (SiO₂, Petroleum ether/Ethyl acetate=1:0 to 1:1) to give (2S)-N-[[2-[2-[tert-butyl(dimethyl)silyl]oxyethyl]-5-ethoxy-4-iodo-pyrazol-3-yl]methyl]-N-ethyl-2-[2-methyl-4-(1-tetrahydropyran-2-yl-3-vinyl-pyrazolo[3,4-c]pyridin-5-yl)pyrazol-3-yl]oxy-propan-1-amine (934 mg, 1.06 mmol, 63% yield, 93% purity) as an orange liquid. ¹H NMR (400 MHz, DMSO-d₆) δ=9.17 (s, 1H), 8.09-8.00 (m, 1H), 7.93-7.84 (m, 1H), 7.08-6.93 (m, 1H), 6.20-6.09 (m, 1H), 6.02-5.92 (m, 1H), 5.60 (d, J=12.0 Hz, 1H), 4.57-4.46 (m, 1H), 4.13-4.05 (m, 3H), 3.34-3.27 (m, 15H), 1.60 (s, 2H), 1.30-1.23 (m, 3H), 1.14-1.08 (m, 3H), 0.99-0.89 (m, 3H), 0.74-0.65 (m, 9H), −0.20 (d, J=1.6 Hz, 6H).

Step 5. To a solution of (2S)-N-[[2-[2-[tert-butyl(dimethyl) silyl]oxyethyl]-5-ethoxy-4-iodo-pyrazol-3-yl]methyl]-N-ethyl-2-[2-methyl-4-(1-tetrahydropyran-2-yl-3-vinyl-pyrazolo [3,4-c]pyridin-5-yl)pyrazol-3-yl]oxy-propan-1-amine (830 mg, 1.01 mmol, 1.00 eq) in DMF (8 mL) was added Pd(OAc)₂ (34.1 mg, 152 mol, 0.15 eq), TBAC (563 mg, 2.03 mmol, 2.00 eq) and KOAc (497 mg, 5.07 mmol, 5.00 eq). The mixture was stirred at 80° C. for 16 h under N₂. On completion, the reaction mixture was diluted with H₂O (150 mL) and extracted with EA (200 mL×3). The combined organic layers were washed with H₂O (80 mL), dried over Na₂SO₄, filtered, and concentrated under reduced pressure to give a residue. The residue was purified by column chromatography (SiO₂, DCM/MeOH=1:0 to 10:1) to give 2-[(10R,17E)-16-ethoxy-12-ethyl-8,10-dimethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (100 mg, 139 μmol, 14% yield, 80% purity) as a red oil. LCMS: (M+1-114: 577.3).

Step 6. To a solution of 2-[(10R,17E)-16-ethoxy-12-ethyl-8,10-dimethyl-2-(oxan-2-yl)-2,8,10,11,12,13-hexahydro-14H-5,3-(azenometheno)tripyrazolol3,4-f:3′,4′-j:4″,3″-n][1,4]oxazacyclopentadecin-14-yl]ethan-1-ol (90.0 mg, 156 μmol, 1.00 eq) in DCM (2 mL) was added TFA (1.54 g, 13.5 mmol, 1.00 mL, 86.3 eq). The mixture was stirred at 25° C. for 3 hr. On completion, the reaction was concentrated in vacuum to give 1-[(10R,17E)-16-ethoxy-12-ethyl-14-(2-hydroxyethyl)-8,10-dimethyl-11,12,13,14-tetrahydro-8H-5,3-(azenometheno)tripyrazolo[3,4-f:3′,4′-j:4″,3″-n][1,4]oxazacyclopentadecin-2(10H)-yl]-2,2,2-trifluoroethan-1-one (90.0 mg, crude) as an orange oil. LCMS: (M+1: 589.2).

Step 7. To a solution of 1-[(10R,17E)-16-ethoxy-12-ethyl-14-(2-hydroxyethyl)-8,10-dimethyl-11,12,13,14-tetrahydro-8H-5,3-(azenometheno)tripyrazolo[3,4-f:3′,4′-j:4″,3″-n][1,4]oxazacyclopentadecin-2(10H)-yl]-2,2,2-trifluoroethan-1-one (90.0 mg, 153 μmol, 1.00 eq) in MeOH (1 mL) was added K₂CO₃ (106 mg, 765 μmol, 5.00 eq). The mixture was stirred at 25° C. for 0.5 hr. On completion, the reaction was concentrated in vacuum to give a residue. The residue was purified by Prep-HPLC purification (column: Phenomenex luna Cis 150*25 mm*10 um; mobile phase: [water (FA)-ACN]; gradient:10%-40% B over 8 min) to give Ex. 6 (19.18 mg, 37.26 μmol, 24% yield, 95.69% purity) as a yellow solid. LCMS: (M+1: 493.2). ¹H NMR for Ex. 6 can be found in the below NMR table.

A byproduct was also obtained as Ex. 7 (2.75 mg, 5.25 μmol, 15% yield, 94.02% purity) as a white solid. LCMS: (M+1: 493.1). ¹H NMR for Ex. 7 can be found in the below NMR table.

### Preparation of 2-[(10S,17E)-16-ethoxy-6,8,10-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 8)

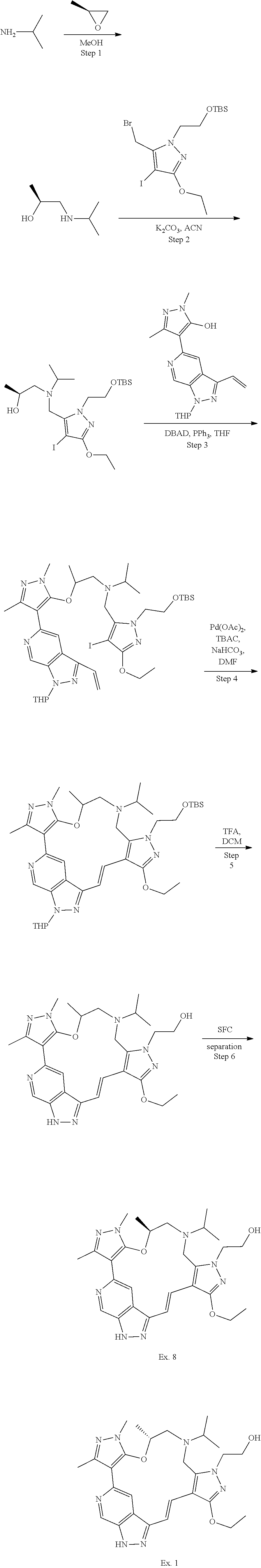

Step 1. To a solution of commercially available propan-2-amine (15.3 g, 258 mmol, 3 eq) in MeOH (80 mL) was added commercially available (2S)-2-methyloxirane (5.00 g, 86.1 mmol, 1 eq). The mixture was stirred at 25° C. for 24 h. On completion, the mixture was concentrated to give (2S)-1-(isopropylamino) propan-2-ol (7.14 g, 60.9 mmol, 71% yield) as a white liquid. ¹H NMR (400 MHz, CDCl₃) δ=3.78-3.65 (m, 1H), 2.83-2.65 (m, 2H), 2.39-2.28 (m, 1H), 1.17-1.09 (m, 3H), 1.08-0.99 (m, 6H).

Step 2. To a solution of (2S)-1-(isopropylamino) propan-2-ol (848 mg, 7.24 mmol, 1.5 eq) in ACN (33 mL) was added K₂CO₃ (1.33 g, 9.65 mmol, 2 eq) and 5-(bromomethyl)-1-(2-((tert-butyldimethylsilyl)oxy)ethyl)-3-ethoxy-4-iodo-1H-pyrazole, which was prepared according to the method described in Ex. 1, (2.36 g, 4.82 mmol, 1 eq). The mixture was stirred at 50° C. for 12 h. On completion, the mixture was filtered and concentrated to give a residue. The residue was purified by column chromatography (SiO₂, PE/THE=1:0 to 5:1) to give (2S)-1-[[2-[2-[tert-butyl(dimethyl) silyl]oxyethyl]-5-ethoxy-4-iodo-pyrazol-3-yl]methyl-isopropylamino]propan-2-ol (2.26 g, 4.30 mmol, 89% yield) as a colorless oil. LCMS: (M+1: 526.6).

Step 3. A mixture of (2S)-1-[[2-[2-[tert-butyl(dimethyl)silyl]oxyethyl]-5-ethoxy-4-iodo-pyrazol-3-yl]methyl-isopropyl-amino]propan-2-ol (650 mg, 1.24 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (420 mg, 1.24 mmol, 1 eq), PPh₃ (649 mg, 2.47 mmol, 2 eq) and DBAD (427 mg, 1.86 mmol, 1.5 eq) was degassed and purged with N₂ for 3 times, and then THE (10 mL) added. The mixture was stirred at 25° C. for 1 h. On completion, the mixture was concentrated to give a residue. The residue was purified by column chromatography (SiO₂, PE:THF=1:1 to 1:1) to give (2R)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-isopropyl-propan-1-amine (781 mg, 922 μmol, 74% yield) as a brown solid. LCMS: (M+1: 847.3).

Step 4. To a solution of (2R)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-isopropyl-propan-1-amine (710 mg, 838 mol, 1 eq) in DMF (35 mL) was added NaHCO₃ (176 mg, 2.10 mmol, 2.5 eq) and TBAC (233 mg, 838 μmol, 1 eq) and Pd(OAc)₂ (37.6 mg, 167 mol, 0.2 eq). The mixture was degassed and purged with N₂ for 3 times, and then the mixture was stirred at 130° C. for 1 h under N₂ atmosphere. On completion, the reaction mixture was partitioned between EA (10 mL×3) and water (30 mL). The combined organic phase was dried over anhydrous sodium sulfate, filtered and concentrated to give a residue. The residue was purified by column chromatography (SiO₂, Petroleum ether/Ethyl acetate=1:1 to 1:1) to give (17E)-14-(2-{[tert-butyl(dimethyl)silyl]oxy}ethyl)-16-ethoxy-6,8,10-trimethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (396 mg, 551 μmol, 66% yield) as a yellow solid. LCMS: (M+1: 719.6).

Step 5. To a solution of (17E)-14-(2-{[tert-butyl(dimethyl)silyl]oxy}ethyl)-16-ethoxy-6,8,10-trimethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (366 mg, 509 μmol, 1 eq) in DCM (3.5 mL) was added TFA (1.87 g, 16.42 mmol, 32.3 eq), the mixture was stirred at 25° C. for 2 h. On completion, the mixture was filtered and concentrated to give a residue. The residue was purified by column chromatography (SiO₂, DCM:MeOH=15:1 to 15:1) to give 2-[(17E)-16-ethoxy-6,8,10-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (40 mg, 76.83 mol, 15% yield) as a brown solid. LCMS: (M+1: 521.3).

Step 6. SFC separation: The product above was separated by SFC (column: DAICEL CHIRALPAK IC(250 mm*30 mm, 10 um); mobile phase: [CO2-ACN/MeOH(0.1% NH3H2O)]; B %:40%, isocratic elution mode) to give Ex. 8 (4.4 mg, 8.45 μmol, 11% yield) as a white solid and Ex. 1 (4.7 mg, 9.03 mol, 11.75% yield) as a white solid. LCMS: (M+1: 521.3). ¹H NMR for Ex. 8 can be found in the below NMR table.

### Preparation of 2-[(10S,17E)-16-ethoxy-8,10-dimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 11) and 2-[(10R,17E)-16-ethoxy-8,10-dimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 12)

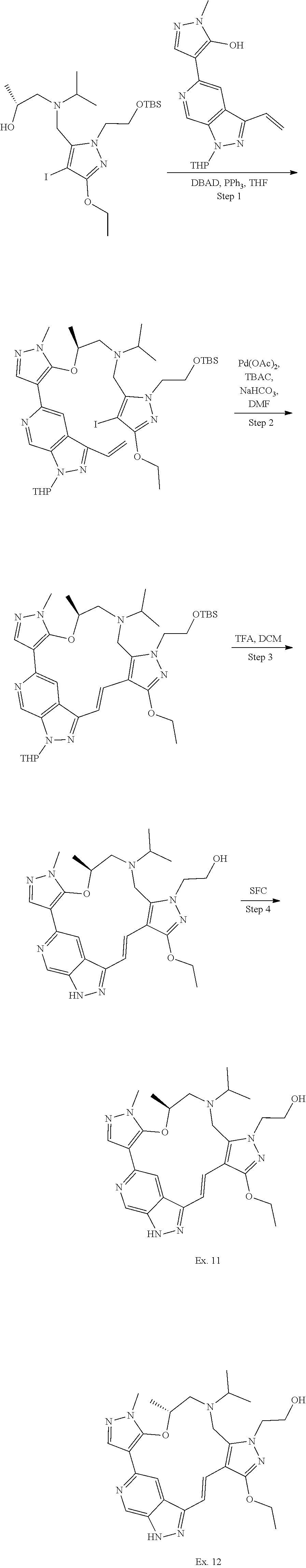

Step 1. To a solution of (2R)-1-[[2-[2-[tert-butyl(dimethyl)silyl]oxyethyl]-5-ethoxy-4-iodo-pyrazol-3-yl]methyl-isopropyl-amino]propan-2-ol, which was prepared according to the method described in Ex. 1, (600 mg, 1.14 mmol, 1 eq), 2-methyl-4-(1-tetrahydropyran-2-yl-3-vinyl-pyrazolo[3,4-c]pyridin-5-yl)pyrazol-3-ol, which was prepared according to the method described in Ex. 5, (371 mg, 1.14 mmol, 1 eq) in THE (10 mL) was added PPh₃ (598 mg, 2.28 mmol, 2 eq) and DBAD (394 mg, 1.71 mmol, 1.5 eq). The mixture was stirred at 25° C. for 2 h. On completion, the mixture was filtered and concentrated to give a residue. The residue was purified by column chromatography (SiO₂, PE/THF=1:1 to 1:1) to give (2S)-N-[[2-[2-[tert-butyl(dimethyl)silyl]oxyethyl]-5-ethoxy-4-iodo-pyrazol-3-yl]methyl]-N-isopropyl-2-[2-methyl-4-(1-tetrahydropyran-2-yl-3-vinyl-pyrazolo[3,4-c]pyridin-5-yl)pyrazol-3-yl]oxy-propan-1-amine (662 mg, 794 mol, 70% yield) as a white solid. LCMS: (M+1:833.2).

Step 2. To a solution of (2S)-N-[[2-[2-[tert-butyl(dimethyl)silyl]oxyethyl]-5-ethoxy-4-iodo-pyrazol-3-yl]methyl]-N-isopropyl-2-[2-methyl-4-(1-tetrahydropyran-2-yl-3-vinyl-pyrazolo[3,4-c]pyridin-5-yl)pyrazol-3-yl]oxy-propan-1-amine (640 mg, 768 μmol, 1 eq), TBAC (213 mg, 768 mol, 1 eq) in DMF (20 mL) was added NaHCO₃ (161 mg, 1.92 mmol, 2.5 eq) and Pd(OAc)₂ (17.2 mg, 76.8 mol, 0.1 eq). The mixture was stirred at 130° C. for 2 h. On completion, the mixture was quenched with water (200 mL) and extracted with ethyl acetate (250 mL×3). The combined organic phase was dried over anhydrous sodium sulfate, filtered, and concentrated to give a residue. The residue was purified by column chromatography (SiO₂, PE/EA=1:1 to 0:1) to give (10S,17E)-14-(2-{1[tert-butyl(dimethyl)silyl]oxy}ethyl)-16-ethoxy-8,10-dimethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (230 mg, 326 μmol, 42% yield) as a yellow solid. LCMS: (M+1: 705.4).

Step 3. To a solution of (10S,17E)-14-(2-{[tert-butyl(dimethyl)silyl]oxy}ethyl)-16-ethoxy-8,10-dimethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (200 mg, 283 μmol, 1 eq) in DCM (0.3 mL) was added TFA (4.60 g, 40.3 mmol, 3.00 mL, 142 eq). The mixture was stirred at 25° C. for 2 h. On completion, the crude product was triturated with ACN (5 mL) at 25° C. for 5 min to give 2-[(10S,17E)-16-ethoxy-8,10-dimethyl-12-(propan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]ethan-1-ol (65 mg, 128 μmol, 45.2% yield) as a white solid. LCMS: (M+1:507.2).

Step 4. SFC separation: the product above was purified by SFC (column: DAICEL CHIRALCEL OD(250 mm*30 mm, 10 um); mobile phase: [CO2-MeOH]; B %:40%, isocratic elution mode) to give Ex. 11 (32.97 mg, 65.08 μmol, 54.95% yield) as a white solid and Ex. 12 (8.41 mg, 16.60 mol, 14.02% yield) as a white solid.

Ex. 11: LCMS: (M+1:507.1). ¹H NMR for Ex. 11 can be found in the below NMR table.

Ex. 12: LCMS: (M+1:507.2). ¹H NMR for Ex. 12 can be found in the below NMR table.

### Preparation of 2-[(10S,17E)-12-ethyl-6,8,10,16-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 13) and 2-[(10R,17E)-12-ethyl-6,8,10,16-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 14)

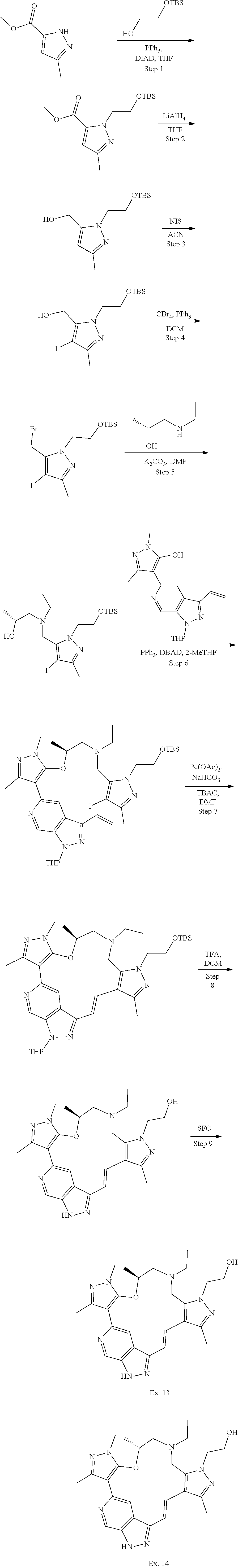

Step 1. The mixture of commercially available methyl 3-methyl-1H-pyrazole-5-carboxylate (20.0 g, 143 mmol, 1 eq), commercially available 2-[tert-butyl(dimethyl)silyl]oxyethanol (50.3 g, 285 mmol, 2 eq) and PPh₃ (82.4 g, 314 mmol, 2.2 eq) in THE (120 mL) was degassed with N₂ and stirred at 25° C. for 30 min under N₂, then DIAD (63.5 g, 314 mmol, 61 mL, 2.2 eq) was added dropwise at 0° C., the resulting mixture was stirred at 25° C. for another 5 h under N₂. On completion, the mixture was triturated with petroleum ether (400 mL) and filtered to remove TPPO. The filtrate was concentrated under reduced pressure to give a residue. The residue was purified by column chromatography (SiO₂, Petroleum ether/Tetrahydrofuran=1/0 to 10/1) to give methyl 2-[2-[tert-butyl (dimethyl) silyl]oxyethyl]-5-methyl-pyrazole-3-carboxylate (40.0 g, 134 mmol, 94% yield) as colorless oil. LCMS: (M+1=299.0).

Step 2. To a solution of methyl 2-[2-[tert-butyl (dimethyl) silyl]oxyethyl]-5-methyl-pyrazole-3-carboxylate (50.0 g, 168 mmol, 1 eq) in THE (400 mL) was added LiAlH₄ (2.5 M, 67.0 mL, 1 eq) at 0° C. The mixture was stirred at 25° C. for 2.5 hr. On completion, the reaction mixture was quenched with water (6 mL) and adjusted with 15% NaOH (6 mL). Then, to the mixture was added water (18 mL) and filtered. The filtrate was concentrated under reduced pressure to give [2-[2-[tert-butyl (dimethyl) silyl]oxyethyl]-5-methyl-pyrazol-3-yl]methanol (45.0 g, 153 mmol, 91% yield, 92% purity) as a colorless oil. LCMS: (M+1=271.1).

Step 3. To a solution of [2-[2-[tert-butyl(dimethyl)silyl]oxyethyl]-5-methyl-pyrazol-3-yl]methanol (45.0 g, 166 mmol, 1 eq) in ACN (300 mL) was added NIS (37.4 g, 166 mmol, 1 eq) at 0° C. The mixture was stirred at 25° C. for 8 hr. On completion, the mixture was quenched with saturated solution of Na₂SO₃(12 mL), and partitioned between water (300 mL) and ethyl acetate(100 mL×3). The combined organic phase was dried over anhydrous sodium sulfate, filtered and concentrated to give a residue. The residue was purified by column chromatography (SiO₂, Petroleum ether/Ethyl acetate=1/1 to 1/2) to give [2-[2-[tert-butyl (dimethyl) silyl]oxyethyl]-4-iodo-5-methyl-pyrazol-3-yl]methanol (10.0 g, 25.2 mmol, 15% yield) as an amber solid. ¹H NMR (400 MHz, DMSO-d₆) δ=5.26 (br s, 1H), 4.46 (s, 2H), 4.24-4.18 (m, 2H), 3.84 (t, J=5.6 Hz, 2H), 2.14-2.04 (m, 3H), 0.80-0.74 (m, 9H), −0.07-0.16 (m, 6H). LCMS: (M+1=397.4).

Step 4. To a solution of [2-[2-[tert-butyl (dimethyl) silyl]oxyethyl]-4-iodo-5-methyl-pyrazol-3-yl]methanol (4.80 g, 12.1 mmol, 1 eq), PPh₃ (3.81 g, 14.5 mmol, 1.2 eq) in DCM (48 mL). Then was added CBr₄ (4.82 g, 14.5 mmol, 1.2 eq) at 0° C. The mixture was stirred at 0° C. for 1 hr. On completion, the mixture was diluted with water (150 mL), extracted with DCM (40 mL×3), dried over Na₂SO₄, and filtered. The filtrate was concentrated to give a residue. The residue was purified by column chromatography (SiO₂, Petroleum ether/Tetrahydrofuran=1/0 to 85/15) to give 2-[5-(bromomethyl)-4-iodo-3-methyl-pyrazol-1-yl]ethoxy-tert-butyl-dimethyl-silane (4.30 g, 9.36 mmol, 77% yield) as a white solid. LCMS: (M+1=460.9).

Step 5. A solution of 2-[5-(bromomethyl)-4-iodo-3-methyl-pyrazol-1-yl]ethoxy-tert-butyl-dimethyl-silane (1.00 g, 2.18 mmol, 1 eq) and commercially available (2R)-1-(ethylamino)propan-2-ol (449 mg, 4.36 mmol, 2 eq.) in DMF (6 mL) was added K₂CO₃ (602 mg, 4.36 mmol, 2 eq). The mixture was stirred at 80° C. for 50 min. On completion, the reaction mixture was partitioned between ethyl acetate (10 mL×3) and water (30 mL). The combined organic phase was dried over anhydrous sodium sulfate, filtered, and concentrated to give a residue. The residue was purified by column chromatography (SiO₂, Petroleum ether/Tetrahydrofuran=4/1 to 3/1) to give (2R)-1-[[2-[2-[tert-butyl (dimethyl) silyl]oxye-thyl]-4-iodo-5-methyl-pyrazol-3-yl]methyl-ethyl-amino]propan-2-ol (1.00 g, 2.08 mmol, 95% yield) as a colorless oil. ¹H NMR (400 MHz, MeOD-d) 6=4.39-4.30 (m, 1H), 4.27-4.19 (m, 1H), 3.89 (br s, 2H), 3.45 (s, 2H), 3.08 (br s, 1H), 2.64-2.59 (m, 1H), 2.37 (br s, 2H), 2.33-2.27 (m, 1H), 2.21 (s, 3H), 1.10-1.03 (m, 6H), 0.78 (s, 9H), −0.11 (d, J=6.4 Hz, 6H). LCMS: (M+1=481.9).

Step 6. A mixture of (2R)-1-[[2-[2-[tert-butyl(dimethyl)silyl]oxyethyl]-4-iodo-5-methyl-pyrazol-3-yl]methyl-ethyl-amino]propan-2-ol (875 mg, 1.62 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (878 mg, 2.59 mmol, 1.6 eq), PPh₃ (933 mg, 3.56 mmol, 2.2 eq), DBAD (819 mg, 3.56 mmol, 2.2 eq) was degassed and purged with N₂ for 3 times, and then to the mixture added 2-MeTHF (9 mL) and stirred at 25° C. for 1.5 hr under N₂ atmosphere. On completion, the mixture was concentrated under reduced pressure to give a residue. The residue was purified by column chromatography (SiO₂, Petroleum ether/Tetrahydrofuran=2/1 to 1/1) to give (2S)-N-∥2-|2-|tert-butyl(dimethyl)silyl]oxyethyl]-4-iodo-5-methyl-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (650 mg, 810 μmol, 5% yield) as colorless solid. ¹H NMR (400 MHz, DMSO-d₆) S=9.23 (s, 1H), 7.93 (d, J=0.8 Hz, 1H), 7.03 (dd, J=12.0, 17.6 Hz, 1H), 6.10 (d, J=18.0 Hz, 1H), 5.97 (dd, J=2.2, 9.6 Hz, 1H), 5.63-5.50 (m, 1H), 4.13-4.07 (m, 2H), 3.94-3.86 (m, 1H), 3.77-3.73 (m, 2H), 3.56 (s, 2H), 3.51 (s, 2H), 3.32 (s, 3H), 2.59 (br d, J=5.2 Hz, 2H), 2.42 (br s, 2H), 2.35-2.31 (m, 2H), 2.26 (s, 3H), 2.06 (s, 3H), 2.02 (br d, J=5.6 Hz, 1H), 1.78-1.70 (m, 1H), 1.61 (br s, 2H), 0.96 (br d, J=6.0 Hz, 3H), 0.83 (br t, J=7.2 Hz, 3H), 0.70 (s, 9H), −0.19 (d, J=1.6 Hz, 6H). LCMS: (M+1=803.6).

Step 7. A mixture of (2S)-N-[[2-[2-[tert-butyl (dimethyl) silyl]oxyethyl]-4-iodo-5-methyl-pyrazol-3-yl]methyl]-2-[2, 5-dimethyl-4-(1-tetrahydropyran-2-yl-3-vinyl-pyrazolo [3,4-c]pyridin-5-yl)pyrazol-3-yl]oxy-N-ethyl-propan-1-amine (650 mg, 810 mol, 1 eq), TBAC (225 mg, 810 μmol, 1 eq), NaHCO₃ (170 mg, 2.02 mmol, 2.5 eq) and Pd(OAc)₂ (36.4 mg, 162 mol, 0.2 eq) in DMF (7 mL) was degassed and purged with N₂ for 3 times, and then the mixture was stirred at 140° C. for 1.5 h under N₂ atmosphere. On completion, the reaction mixture was partitioned between ethyl acetate (30 mL×3) and water (90 mL). The combined organic phase was dried over anhydrous sodium sulfate, filtered and concentrated to give a residue. The residue was purified by column chromatography (SiO₂, Petroleum ether/Tetrahydrofuran=1/1 to 1/2) to give (10S,17E)-14-(2-{[tert-butyl(dimethyl)silyl]oxy}ethyl)-12-ethyl-6,8,10,16-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (210 mg, 311 μmol, 38% yield) as a brown solid. LCMS: (M+1 =675.4).

Step 8. To a solution of (10S,17E)-14-(2-{[tert-butyl(dimethyl)silyl]oxy}ethyl)-12-ethyl-6,8,10,16-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (200 mg, 296 μmol, 1 eq) was added DCM (2.5 mL) and TFA (0.5 mL). The mixture was stirred at 25° C. for 1 hr. On completion, the mixture was concentrated under reduced pressure to give a residue. The residue was purified by Prep-HPLC purification (column: Welch Xtimate C18 150*25 mm*5 um; mobile phase: [water (TFA)-ACN]; gradient: 10%-40% B over 10 min) to give 2-[(10S,17E)-12-ethyl-6,8,10,16-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (50.0 mg, 105 mol, 35% yield) as a yellow solid. ¹H NMR (400 MHz, MeOD-d₄) S=9.11 (d, J=0.8 Hz, 1H), 8.62 (s, 1H), 7.66 (br d, J=17.2 Hz, 1H), 7.20 (d, J=16.8 Hz, 1H), 5.37-5.26 (m, 1H), 4.93 (br s, 1H), 4.71-4.63 (m, 1H), 4.39-4.26 (m, 2H), 3.99 (br d, J=4.0 Hz, 1H), 3.94-3.83 (m, 2H), 3.74 (s, 3H), 3.47 (hr d, J=14.4 Hz, 2H), 3.39-3.33 (m, 1H), 2.56 (d, J=12.4 Hz, 6H), 1.46 (br t, J=6.4 Hz, 3H), 1.19 (d, J=6.4 Hz, 3H). LCMS: (M+1=477.3).

Step 9. SFC separation: the residue was separated by SFC (column: DAICEL CHIRALCEL OD (250 mm*30 mm, 10 um); mobile phase: [CO2-MeOH(0.1% NH₃H2O)]; B %:40%, isocratic elution mode) to give Ex. 13 (5.93 mg, 12.4 mol, 12% yield, 100% purity) as a white solid and Ex. 14 (5.93 mg, 12.44 μmol, 11.86% yield, 100% purity) as a white solid.

Ex. 13: LCMS: (M+1=477.1). ¹H NMR for Ex. 13 can be found in the below NMR table.

Ex. 14: LCMS: (M+1=477.1). ¹H NMR for Ex. 14 can be found in the below NMR table.

### Preparation of 2-[(10S,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 15)

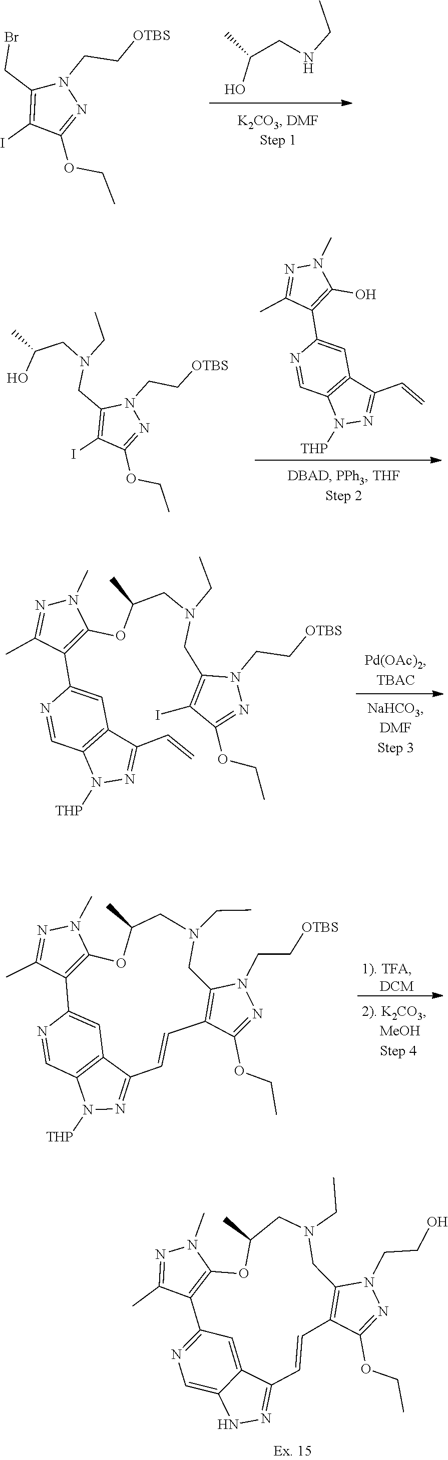

Step 1. To a solution of 2-[5-(bromomethyl)-3-ethoxy-4-iodo-pyrazol-1-yl]ethoxy-tert-butyl-dimethyl-silane, prepared according to the method described in Ex. 1, (539 mg, 1.10 mmol, 1 eq), K₂CO₃ (304 mg, 2.20 mmol, 2 eq) in DMF (5 mL) was added commercially available (2R)-1-(ethylamino)propan-2-ol (170 mg, 1.65 mmol, 1.5 eq). The mixture was stirred at 80° C. for 1 h. On completion, the resulting mixture was diluted with H₂O (20 mL) and extracted with EtOAc (3×20 mL). The combined organic layers were washed with brine (3×20 mL), dried over Na₂SO₄ and concentrated in vacuum. The residue was purified by column chromatography (SiO₂, Petroleum ether/EA=1/0 to 1/1) to give (2R)-1-[[2-[2-[tert-butyl(dimethyl)silyl]oxyethyl]-5-ethoxy-4-iodo-pyrazol-3-yl]methyl-ethyl-amino]propan-2-ol (500 mg, 918 μmol, 83% yield, 94% purity) as a yellow oil. ¹H NMR (400 MHz, CDCl₃) δ 7.27 (s, 1H), 4.26 (q, J=7.2 Hz, 3H), 4.15 (d, J=13.6 Hz, 1H), 3.91-3.74 (m, 4H), 3.50 (d, J=13.6 Hz, 1H), 3.09 (s, 1H), 2.73-2.58 (m, 1H), 2.55-2.40 (m, 2H), 2.38-2.24 (m, 1H), 1.47-1.36 (m, 4H), 1.13-1.06 (m, 4H), 0.81 (s, 9H), −0.07 (d, J=6.0 Hz, 6H). LCMS: (M+1: 512.1).

Step 2. A mixture of (2R)-1-[[2-[2-[tert-butyl(dimethyl)silyl]oxyethyl]-5-ethoxy-4-iodo-pyrazol-3-yl]methyl-ethyl-amino]propan-2-ol (457 mg, 893 μmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (606 mg, 1.79 mmol, 2 eq), DBAD (452 mg, 1.97 mmol, 2.2 eq), PPh₃ (515 mg, 1.97 mmol, 2.2 eq) in THE (10 mL) was stirred at 25° C. for 1 h. On completion, the mixture was concentrated in vacuum. The residue was purified by column chromatography (SiO₂, Petroleum ether/THF=1/0 to 5/1) to give (2S)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (426 mg, 347 mol, 39% yield, 68% purity) as a yellow solid. ¹H NMR (400 MHz, DMSO-d₆) δ 9.23 (s, 1H), 7.93 (s, 1H), 6.10 (d, J=17.2 Hz, 1H), 5.97 (dd, J=2.0, 9.6 Hz, 1H), 5.58 (d, J=11.2 Hz, 1H), 4.11-4.02 (m, 4H), 3.95-3.85 (m, 2H), 3.83-3.68 (m, 4H), 3.57 (s, 3H), 3.48 (s, 2H), 2.69-2.59 (m, 3H), 2.33 (s, 3H), 2.08-2.00 (m, 3H), 1.61 (s, 3H), 1.27 (t, J=7.2 Hz, 3H), 1.17 (t, J=7.2 Hz, 2H), 0.96 (d, J=6.0 Hz, 2H), 0.83 (t, J=7.2 Hz, 3H), 0.71 (s, 9H), −0.18 (d, J=1.6 Hz, 6H). LCMS: (M+1: 833.1).

Step 3. To a solution of (2S)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (400 mg, 480 mol, 1 eq), NaHCO₃ (100 mg, 1.20 mmol, 2.5 eq) and Pd(OAc)₂ (21.5 mg, 96.1 mol, 0.2 eq) in DMF (35 mL) was added TBAC (133 mg, 480 mol, 1 eq) at 25° C. The mixture was stirred at 140° C. for 1 h under nitrogen atmosphere. On completion, the mixture was added brine (100 mL) and extracted with EA (50 mL×3). The combined organic layer was washed with brine (50 mL×2). The organic layer was concentrated in vacuum to give the residue. The residue was purified by column chromatography (SiO₂, DCM:MeOH=100:1~30:1) to give (10S,17E)-14-(2-{[tert-butyl(dimethyl)silyl]oxy}ethyl)-16-ethoxy-12-ethyl-6,8,10-trimethyl-2-(oxan-2-yl)-2,10,11,12,13,14-hexahydro-8H-5,3-(azenometheno)tripyrazolo|3,4-f:3′,4′-j:4″,3″-n][1,4]oxazacyclopentadecine (180 mg, 209 mol, 44% yield, 82% purity) as a yellow solid. ¹H NMR (400 MHz, DMSO-d) δ 9.16 (s, 1H), 8.47 (s, 1H), 8.08 (d, J=17.8 Hz, 1H), 7.10 (d, J=16.8 Hz, 1H), 5.91 (d, J=9.6 Hz, 1H), 4.35-4.25 (m, 2H), 4.10 (s, 2H), 3.96-3.82 (m, 6H), 3.63 (s, 3H), 2.62-2.58 (m, 2H), 2.33 (s, 4H), 2.09-1.95 (m, 3H), 1.80-1.70 (m, 1H), 1.60 (d, J=2.4 Hz, 3H), 1.42 (t, J=7.2 Hz, 4H), 1.10-1.03 (m, 3H), 0.91 (t, J=7.2 Hz, 3H), 0.79 (s, 9H), −0.07 (s, 6H). LCMS: (M+1: 705.3).

Step 4. To a solution of (10S,17E)-14-(2-{[tert-butyl(dimethyl)silyl]oxy}ethyl)-16-ethoxy-12-ethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (150 mg, 144 μmol, 1 eq) in DCM (2 mL) was added TFA (3.54 g, 31.0 mmol, 2.31 mL, 214 eq) at 25° C. The mixture was stirred at 25° C. for 1.5 h. On completion, the mixture was concentrated to give a yellow oil (103 mg, 105 μmol, 73% yield, 73% purity, TFA salt). LCMS: (M+1: 603.2). This yellow oil (103 mg, 144 μmol, 1 eq, TFA) was dissolved in MeOH (2 mL) and K₂CO₃ (199 mg, 1.45 mmol, 10 eq) was added. The mixture was stirred at 25° C. for 1 h. On completion, the mixture was filtered and concentrated in vacuum. The residue was purified by silica gel column chromatography (4 g silica gel column, MeOH in DCM from 0% to 20%) to give 2-[(10S,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (50.0 mg, 98.7 mol, 68% yield) as a white solid, which is further purified by Prep-SFC (column: DAICEL CHIRALCEL OD (250 mm*30 mm, 10 um); mobile phase: [CO₂-MeOH(0.1% NH₃H₂O)]; B %:30%, isocratic elution mode) to give Ex. 15 (25.01 mg, 47.39 μmol, 48.02% yield, 96% purity) as an off-white solid. LCMS: (M+1: 507.2). ¹H NMR for Ex. 15 can be found in the below NMR table.

### Preparation of 2-[(11S,17E)-16-ethoxy-12-ethyl-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 16) and 2-[(10R,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 17)

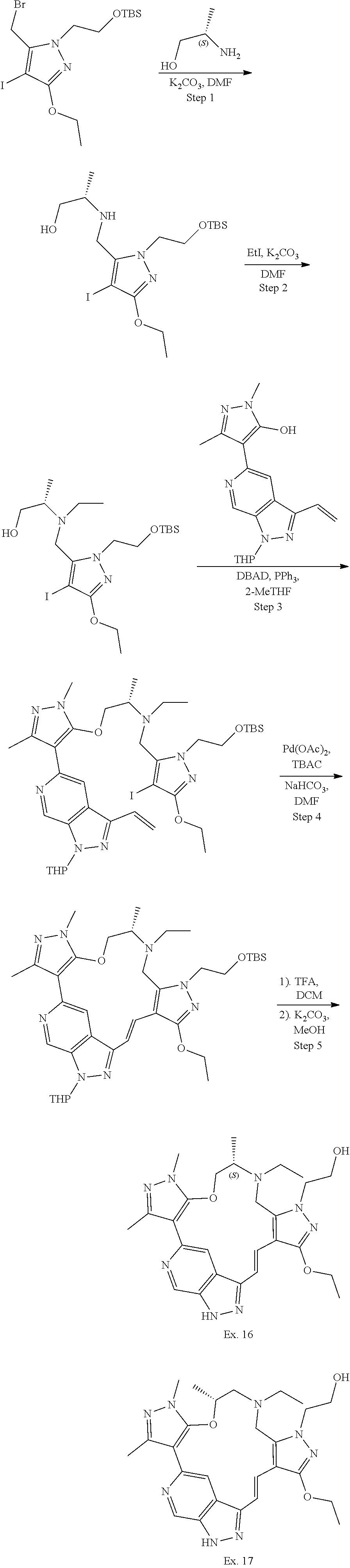

Step 1. To a solution of commercially available (2S)-2-aminopropan-1-ol (553 mg, 7.36 mmol, 3 eq) and 5-(bromomethyl)-1-(2-((tert-butyldimethylsilyl)oxy)ethyl)-3-ethoxy-4-iodo-1H-pyrazole, which was prepared according to the method described in Ex. 1, (1.20 g, 2.45 mmol, 1 eq) in DMF (15 mL) was added K₂CO₃ (1.02 g, 7.36 mmol, 3 eq). The mixture was stirred at 70° C. for 2 h. On completion, the crude mixture was used for next step. (2S)-2-[[2-[2-[tert-butyl (dimethyl) silyl]oxyethyl]-5-ethoxy-4-iodo-pyrazol-3-yl]methylamino]propan-1-ol (1.19 g, 2.46 mmol, 100% yield) was obtained as colorless oil. LCMS: (M+1=484.3).

Step 2. To a mixture of (2S)-2-[[2-[2-[tert-butyl(dimethyl)silyl]oxyethyl]-5-ethoxy-4-iodo-pyrazol-3-yl]methylamino]propan-1-ol (1.19 g, 2.46 mmol, 1 eq) and EtI (1.15 g, 7.38 mmol, 3 eq) in DMF (15 mL) was added K₂CO₃ (1.02 g, 7.38 mmol, 3 eq). The mixture was stirred at 70° C. for 2 h. On completion, the mixture was diluted with water (50 mL) and extracted with EtOAc (30 mL×3). The combined organic layer was dried over anhydrous Na₂SO₄, filtered and the filtrate was concentrated in vacuum to give crude. The residue was purified by CombiFlash chromatography (20 g silica gel column, EtOAc in PE from 0% to 100%). (2S)-2-[[2-[2-[tert-butyl(dimethyl)silyl]oxyethyl]-5-ethoxy-4-iodo-pyrazol-3-yl]methyl-ethyl-amino]propan-1-ol (940 mg, 1.84 mmol, 74% yield) was obtained as yellow oil. ¹H NMR (400 MHz, CDCl₃) δ=4.43-4.31 (m, 1H), 4.29-4.20 (m, 2H), 4.15-4.03 (m, 1H), 3.97-3.80 (m, 3H), 3.56-3.45 (m, 1H), 3.43-3.29 (m, 2H), 3.01-2.79 (m, 2H), 2.69-2.55 (m, 1H), 2.41-2.24 (m, 1H), 1.46-1.38 (m, 3H), 1.12 (t, J=6.8 Hz, 3H), 0.97 (d, J=6.4 Hz, 3H), 0.87-0.75 (m, 9H), −0.01-0.15 (m, 6H).

Step 3. A solution of (2S)-2-[[2-[2-[tert-butyl(dimethyl)silyl]oxyethyl]-5-ethoxy-4-iodo-pyrazol-3-yl]methyl-ethyl-amino]propan-1-ol (900 mg, 1.76 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (744 mg, 2.29 mmol, 1.3 eq), PPh₃ (1.02 g, 3.87 mmol, 2.2 eq), DBAD (891 mg, 3.87 mmol, 2.2 eq) in 2-MeTHF (10 mL) was stirred at 25° C. for 2 h. On completion, the mixture was concentrated in vacuum to give crude. The residue was purified by CombiFlash chromatography (20 g silica gel, EtOAc in PE from 0% to 100%). (2S)-N-[[2-[2-[tert-butyl(dimethyl)silyl]oxyethyl]-5-ethoxy-4-iodo-pyrazol-3-yl]methyl]-1-[2,5-dimethyl-4-(1-tetrahydropyran-2-yl-3-vinyl-pyrazolo[3,4-c]pyridin-5-yl)pyrazol-3-yl]oxy-N-ethyl-propan-2-amine (1.00 g, 1.20 mmol, 68% yield) was obtained as yellow solid. LCMS: (M+1=833.7).

Step 4. To a solution of (2S)-N-[[2-[2-[tert-butyl(dimethyl)silyl]oxyethyl]-5-ethoxy-4-iodo-pyrazol-3-yl]methyl]-1-[2,5-dimethyl-4-(1-tetrahydropyran-2-yl-3-vinyl-pyrazolo[3,4-c]pyridin-5-yl)pyrazol-3-yl]oxy-N-ethyl-propan-2-amine (980 mg, 1.18 mmol, 1 eq), TBAC (327 mg, 1.18 mmol, 1 eq), NaHCO₃ (247 mg, 2.94 mmol, 2.5 eq) and Pd(OAc)₂ (52.8 mg, 235 mol, 0.2 eq) in DMF (10 mL) was stirred at 140° C. for 1 h. On completion, the mixture was diluted with water (50 mL) and extracted with EtOAc (15 mL×3). The combined organic layer was dried over anhydrous Na₂SO₄, filtered and the filtrate was concentrated in vacuum to give crude. The residue was purified by CombiFlash chromatography (20 g silica gel column, EtOAc in PE from 0% to 100%). (11S,17E)-14-(2-{[tert-butyl(dimethyl)silyl]oxy}ethyl)-16-ethoxy-12-ethyl-6,8,11-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (540 mg, 766 μmol, 65% yield) was obtained as brown oil. LCMS: (M+1=705.4).

Step 5. To a solution of (11S,17E)-14-(2-{[tert-butyl(dimethyl)silyl]oxy}ethyl)-16-ethoxy-12-ethyl-6,8,11-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (510 mg, 723 μmol, 1 eq) in DCM (6 mL) was added TFA (1.84 g, 16.2 mmol, 1.2 mL, 22.3 eq). The mixture was stirred at 25° C. for 2 h. On completion, the mixture was concentrated in vacuum to give a yellow oil (380 mg, 614 μmol, 85% yield). LCMS: (M+1=603.3). This yellow oil (380 mg, 614 mol, 1 eq) was dissolved in MeOH (6 mL) and K₂CO₃ (424 mg, 3.07 mmol, 5 eq) was added. The mixture was stirred at 25° C. for 2 h. On completion, the mixture was filtered, and the filtrate was concentrated in vacuum to give crude. The residue was purified by Prep-HPLC (column: Welch Xtimate C18 150*25 mm*5 um; mobile phase: [water (TFA)-ACN]; gradient: 13%-43% B). Ex. 16 (19.78 mg, 36.92 μmol, 6.01% yield, 94.55% purity) was obtained as orange solid and Ex. 17 (3.03 mg, 5.61 mol, 9.13e−1% yield, 93.79% purity) was obtained as white solid as a biproduct.

Ex. 16: LCMS: (M+1=507.3). ¹H NMR for Ex. 16 can be found in the below NMR table.

Ex. 17: LCMS: (M+1=507.3). ¹H NMR for Ex. 17 can be found in the below NMR table.

### Preparation of 2-[(11R,17E)-16-ethoxy-12-ethyl-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 18)

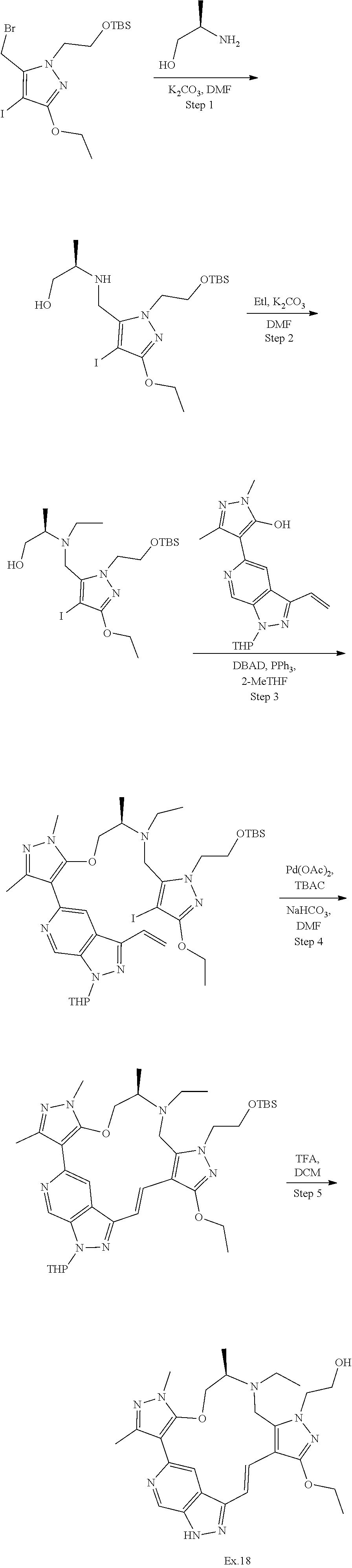

Step 1. To a solution of 2-[5-(bromomethyl)-3-ethoxy-4-iodo-pyrazol-1-yl]ethoxy-tert-butyl-dimethyl-silane, prepared according to the method described in Ex. 1, (1.20 g, 2.45 mmol, 1.00 eq) and commercially available (2R)-2-aminopropan-1-ol (552 mg, 7.36 mmol, 573 μL, 3.00 eq) in DMF (12 mL) was added K₂CO₃ (1.02 g, 7.36 mmol, 3.00 eq). The mixture was stirred at 70° C. for 2 h. On completion, compound (2R)-2-[[2-[2-[tert-butyl(dimethyl)silyl]oxyethyl]-5-ethoxy-4-iodo-pyrazol-3-yl]methylami-no]propan-1-ol (1.00 g, in DMF) was obtained as a colorless liquid.

Step 2. To a solution of (2R)-2-[[2-[2-[tert-butyl(dimethyl)silyl]oxyethyl]-5-ethoxy-4-iodo-pyrazol-3-yl]methylamino]propan-1-ol (1.00 g, in DMF) and iodoethane (1.29 g, 8.27 mmol, 4.00 eq) in DMF (12 mL) was added K₂CO₃ (572 mg, 4.14 mmol, 2.00 eq). The mixture was stirred at 70° C. for 2 h. On completion, the mixture was quenched with water (50 mL) and extracted with ethyl acetate (25 mL×3). The combined organic phase was dried over anhydrous sodium sulfate, filtered, and concentrated to give a residue. The residue was purified by column chromatography (SiO₂, Petroleum ether/Ethyl acetate=3:1 to 1:1) to give (2R)-2-[[2-[2-[tert-butyl(dimethyl)silyl]oxyethyl]-5-ethoxy-4-iodo-pyrazol-3-yl]methyl-ethyl-amino]propan-1-ol (800 mg, 1.49 mmol, 72% yield, 95% purity) as a colorless oil. LCMS: (M+1=512.4).

Step 3. A mixture of (2R)-2-[[2-[2-[tert-butyl(dimethyl)silyl]oxyethyl]-5-ethoxy-4-iodo-pyrazol-3-yl]methyl-ethyl-amino]propan-1-ol (750 mg, 1.47 mmol, 1.00 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (647 mg, 1.91 mmol, 1.30 eq), PPh₃ (846 mg, 3.23 mmol, 2.20 eq), DBAD (743 mg, 3.23 mmol, 2.20 eq) was degassed and purged with N₂ for 3 times. Then 2-MeTHF (7.5 mL) was added and the mixture was stirred at 25° C. for 2 h under N₂ atmosphere. On completion, the reaction mixture was concentrated under reduced pressure to remove solvent. The residue was purified by column chromatography (SiO₂, Petroleum ether/THF=1:0 to 0:1) to give (2R)-N-[[2-[2-[tert-butyl(dimethyl)silyl]oxyethyl]-5-ethoxy-4-iodo-pyrazol-3-yl]methyl]-1-[2,5-dimethyl-4-(1-tetrahydropyran-2-yl-3-vinyl-pyrazolo[3,4-c]pyridin-5-yl)pyrazol-3-yl]oxy-N-ethyl-propan-2-amine (840 mg, 917 μmol, 63% yield, 91% purity) as a colorless oil. LCMS: (M+1=833.3).

Step 4. A mixture of (2R)-N-[[2-[2-[tert-butyl(dimethyl)silyl]oxyethyl]-5-ethoxy-4-iodo-pyrazol-3-yl]methyl]-1-[2,5-dimethyl-4-(1-tetrahydropyran-2-yl-3-vinyl-pyrazolo[3,4-c]pyridin-5-yl)pyrazol-3-yl]oxy-N-ethyl-propan-2-amine (790 mg, 949 mol, 1.00 eq), diacetoxypalladium (42.6 mg, 189 mol, 0.20 eq), TBAC (264 mg, 949 mol, 1.00 eq) and NaHCO₃ (199 mg, 2.37 mmol, 2.50 eq) in DMF (1 mL) was degassed and purged with N₂ for 3 times, and then the mixture was stirred at 120° C. for 1 h under N₂ atmosphere. On completion, the mixture was quenched with water (40 mL) and extracted with ethyl acetate (25 mL×3). The combined organic phase was dried over anhydrous sodium sulfate, filtered and concentrated to give a residue. The residue was purified by column chromatography (SiO₂, Petroleum ether/Ethyl acetate=1:1 to 1:1) to give (11R,17E)-14-(2-{[tert-butyl(dimethyl)silyl]oxy}ethyl)-16-ethoxy-12-ethyl-6,8,11-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (530 mg, 752 μmol, 79% yield) as a brown oil. LCMS: (M+1=705.6).

Step 5. To a solution of (11R,17E)-14-(2-{[tert-butyl(dimethyl)silyl]oxy}ethyl)-16-ethoxy-12-ethyl-6,8,11-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (130 mg, 184 μmol, 1.00 eq) in DCM (1 mL) was added TFA (1.54 g, 13.5 mmol, 73.0 eq). The mixture was stirred at 25° C. for 2 h. On completion, the mixture was concentrated to give a residue. The residue was purified by Prep-HPLC (column: Phenomenex luna C18 150*25 mm*10 um; mobile phase: [water (FA)-ACN]; gradient:12%-42% B over 15 min) to give Ex. 18 (15.49 mg, 27.75 mol, 15.05% yield, 99% purity, FA) as a white solid. LCMS: (M+1=507.2). ¹H NMR for Ex. 18 can be found in the below NMR table.

### Preparation of (2S)-2-[(10S,17E)-16-ethoxy-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 19)

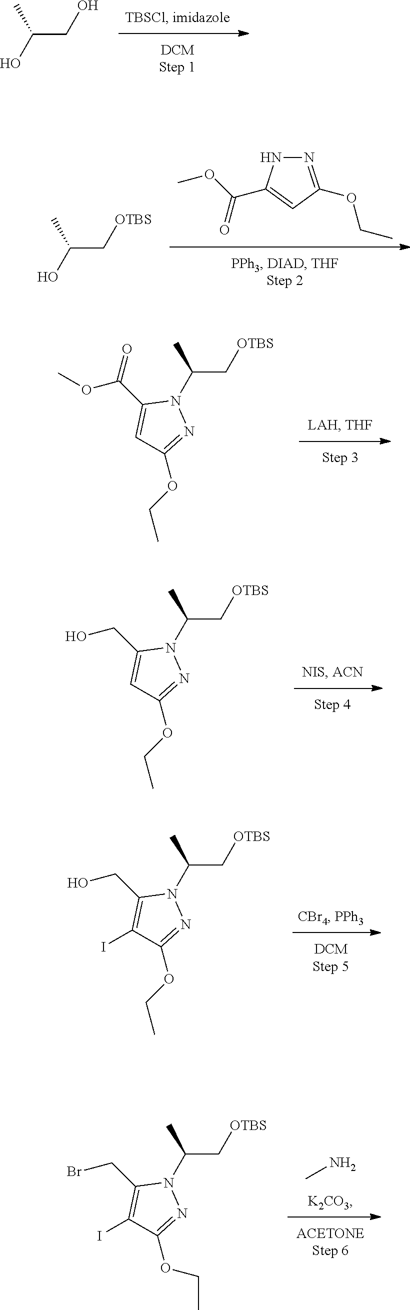

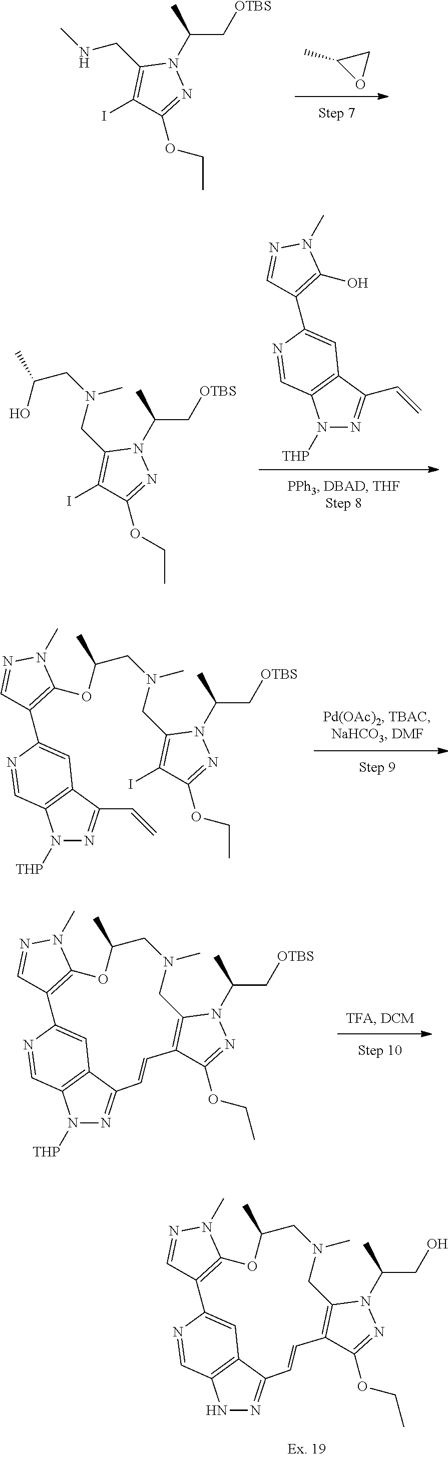

Step 1. To a solution of commercially available (2R)-propane-1,2-diol (50.0 g, 657 mmol, 48.0 mL, 1 eq) in DCM (500 mL) was added TBSCl (99.0 g, 657 mmol, 1 eq) and imidazole (44.7 g, 657 mmol, 1 eq). The mixture was stirred at 0° C. for 2 h and then at 25° C. for 10 hr. On completion, the mixture was filtered and concentrated to give (2R)-1-[tert-butyl(dimethyl)silyl]oxypropan-2-ol (120 g, 630 mmol, 95% yield) as colorless oil. ¹H NMR (400 MHz, DMSO-d₆) δ=4.75-4.22 (m, 1H), 3.64-3.53 (m, 1H), 3.47 (dd, J=5.6, 9.6 Hz, 1H), 3.31-3.23 (m, 1H), 1.06-0.97 (m, 3H), 0.92-0.84 (m, 9H), 0.02 (s, 6H).

Step 2. A mixture of methyl 3-ethoxy-1H-pyrazole-5-carboxylate, which was prepared according to the method described in Ex. 1, (22.0 g, 129 mmol, 1 eq), (2R)-1-[tert-butyl(dimethyl)silyl]oxypropan-2-ol (49.2 g, 258 mmol, 2 eq), PPh₃ (74.6 g, 284 mmol, 2.2 eq) in THE (250 mL) was degassed and purged with N₂ for 3 times, and then the mixture was stirred at 25° C. for 0.5 h. Then DIAD (57.5 g, 284 mmol, 55.1 mL, 2.2 eq) was added at 0° C., and then the mixture was stirred at 25° C. for 2 h. On completion, the mixture was concentrated in vacuum to give a residue, then added Petroleum ether/Ethyl acetate=5:1 (200 mL) and was stirred at 25° C. for 10 min. Then the mixture was filtered and concentrated to give a residue. The residue was purified by column chromatography (SiO₂, Petroleum ether/Ethyl acetate=1:0 to 5:1) to give methyl 2-[(1S)-2-[tert-butyl (dimethyl) silyl]oxy-1-methyl-ethyl]-5-ethoxy-pyrazole-3-carboxylate (36.0 g, 105 mmol, 81% yield) as transparent oil. ¹H NMR (400 MHz, CDCl₃-d) 6=6.15 (s, 1H), 5.47-5.33 (m, 1H), 4.15 (dq, J=1.2, 7.2 Hz, 2H), 3.87-3.71 (m, 5H), 1.44-1.36 (m, 6H), 0.80 (s, 9H), −0.03-0.12 (m, 6H).

Step 3. To a solution of methyl 2-[(1S)-2-[tert-butyl(dimethyl)silyl]oxy-1-methyl-ethyl]-5-ethoxy-pyrazole-3-carboxylate (36.0 g, 105 mmol, 1 eq) in THE (300 mL) was added LAH (2.5 M, 63.0 mL, 1.5 eq) at 0° C. The mixture was stirred at 25° C. for 2 h. On completion, the reaction mixture was quenched with H₂O (6 mL) and adjusted with 15% NaOH (6 mL). The combined organic layers were dried over Na₂SO₄ with stirring 10 min at 25° C., filtered and concentrated under reduced pressure to give [2-[(1S)-2-[tert-butyl(dimethyl)silyl]oxy-1-methyl-ethyl]-5-ethoxy-pyrazol-3-yl]methanol (30.0 g, crude) as transparent oil. LCMS: (M+1:315.3).

Step 4. To a solution of [2-[(1S)-2-[tert-butyl(dimethyl)silyl]oxy-1-methyl-ethyl]-5-ethoxy-pyrazol-3-yl]methanol (29.5 g, 93.8 mmol, 1 eq) in ACN (400 mL) was added NIS (21.1 g, 93.8 mmol, 1 eq). The mixture was stirred at 0° C. for 2 h. On completion, the reaction mixture was quenched dropwise with sodium sulfite aqueous solution (300 mL), then was extracted with EA (200 mL×3). The combined organic layer was washed with brine (200 mL), dried over Na₂SO₄, filtered, and concentrated to give a residue. The residue was purified by column chromatography (SiO₂, Petroleum ether/Ethyl acetate=5:1 to 3:1) to give [2-[(1S)-2-[tert-butyl(dimethyl)silyl]oxy-1-methyl-ethyl]-5-ethoxy-4-iodo-pyrazol-3-yl]methanol (25.6 g, 58.1 mmol, 61.9% yield) as white solid. ¹H NMR (400 MHz, DMSO-d₆) δ=5.28 (dd, J=4.4, 6.0 Hz, 1H), 4.56-4.43 (m, 2H), 4.34 (dd, J=4.4, 13.2 Hz, 1H), 4.20-4.09 (m, 2H), 3.67 (d, J=6.8 Hz, 2H), 1.33-1.25 (m, 6H), 0.75 (s, 9H), −0.07 (s, 3H), −0.17 (s, 3H).

Step 5. To a solution of [2-[(1S)-2-[tert-butyl(dimethyl)silyl]oxy-1-methyl-ethyl]-5-ethoxy-4-iodo-pyrazol-3-yl]methanol (2.00 g, 4.54 mmol, 1 eq) in DCM (20 mL) was added PPh₃ (1.43 g, 5.45 mmol, 1.2 eq), and then CBr₄ (1.81 g, 5.45 mmol, 1.2 eq) was added at 0° C. under N₂. The mixture was stirred at 25° C. for 1 h. On completion, the mixture was concentrated to give a residue. The residue was purified by column chromatography (SiO₂, PE:THF=1:0 to 5:1) to give [(2S)-2-[5-(bromomethyl)-3-ethoxy-4-iodo-pyrazol-1-yl]propoxy]-tert-butyl-dimethyl-silane (1.7 g, 3.38 mmol, 74% yield) as a yellow oil. LCMS: (M+1:504.7).

Step 6. To a solution of MeNH₂ (10.2 g, 99.3 mmol, 30% purity, 20 eq) in acetone (150 mL) was added K₂CO₃ (2.06 g, 14.9 mmol, 3 eq). And then [(2S)-2-[5-(bromomethyl)-3-ethoxy-4-iodo-pyrazol-1-yl]propoxy|-tert-butyl-dimethyl-silane (2.50 g, 4.97 mmol, 1 eq) in acetone (150 mL) was added dropwise at 0° C. The mixture was stirred at 0° C. for 2 h. On completion, the mixture was filtered and concentrated to give a residue. The residue was purified by column chromatography (SiO₂, PE/THF=1:1 to 1:1) to give 1-[2-[(1S)-2-[tert-butyl(dimethyl)silyl]oxy-1-methyl-ethyl]-5-ethoxy-4-iodo-pyrazol-3-yl]-N-methyl-methanamine (1.83 g, 4.04 mmol, 81% yield) as a yellow oil. LCMS: (M+1:454.0).

Step 7. To a solution of 1-[2-[(1S)-2-[tert-butyl(dimethyl)silyl]oxy-1-methyl-ethyl]-5-ethoxy-4-iodo-pyrazol-3-yl]-N-methyl-methanamine (1.70 g, 3.75 mmol, 1 eq), commercially available (2R)-2-methyloxirane (1.09 g, 18.7 mmol, 5 eq) in EtOH (20 mL). The mixture was stirred at 80° C. for 2 h. On completion, the mixture was concentrated to give a residue. The residue was purified by column chromatography (SiO₂, Petroleum ether/THF=2:1 to 1:1) to give (2R)-1-[[2-[(1S)-2-[tert-butyl(dimethyl)silyl]oxy-1-methyl-ethyl]-5-ethoxy-4-iodo-pyrazol-3-yl]methyl-methyl-amino]propan-2-ol (1.90 g, 3.71 mmol, 99% yield) as a yellow oil. LCMS: (M+1:512.5).

Step 8. To a solution of (2R)-1-[[2-[(1S)-2-[tert-butyl(dimethyl)silyl]oxy-1-methyl-ethyl]-5-ethoxy-4-iodo-pyrazol-3-yl]methyl-methyl-amino]propan-2-ol (600 mg, 1.17 mmol, 1 eq), 2-methyl-4-(1-tetrahydropyran-2-yl-3-vinyl-pyrazolo[3,4-c]pyridin-5-yl)pyrazol-3-ol, which was prepared according to the method described in Ex. 5, (382 mg, 1.17 mmol, 1 eq) in THE (15 mL) was added PPh₃ (615 mg, 2.35 mmol, 2 eq) and DBAD (405 mg, 1.76 mmol, 1.5 eq). The mixture was stirred at 25° C. for 2 h under N₂. On completion, the mixture was concentrated to give a residue. The residue was purified by column chromatography (SiO₂, PE/THF) to give (2S)-N-[[2-[(1S)-2-[tert-butyl(dimethyl)silyl]oxy-1-methyl-ethyl]-5-ethoxy-4-iodo-pyrazol-3-yl]methyl]-N-methyl-2-[2-methyl-4-(1-tetrahydropyran-2-yl-3-vinyl-pyrazolo[3,4-c]pyridin-5-yl)pyrazol-3-yl]oxy-propan-1-amine (700 mg, 854 mol, 73% yield) as a white solid. LCMS: (M+1: 819.3).

Step 9. To a solution of (2S)-N-[[2-[(1S)-2-[tert-butyl(dimethyl)silyl]oxy-1-methyl-ethyl]-5-ethoxy-4-iodo-pyrazol-3-yl]methyl]-N-methyl-2-[2-methyl-4-(1-tetrahydropyran-2-yl-3-vinyl-pyrazolo[3,4-c]pyridin-5-yl)pyrazol-3-yl]oxy-propan-1-amine (680 mg, 830 mol, 1 eq), TBAC (230 mg, 830 mol, 1 eq) in DMF (70 mL) was added Pd(OAc)₂ (18.6 mg, 83.0 μmol, 0.1 eq) and NaHCO₃ (174 mg, 2.08 mmol, 2.5 eq). The mixture was stirred at 100° C. for 2 h under N₂ atmosphere. On completion, the mixture was quenched with water (20 mL) and extracted with ethyl acetate (25 mL×3). The combined organic phase was dried over anhydrous sodium sulfate, filtered and concentrated to give a residue. The residue was purified by column chromatography (SiO₂, PE/EA=1:1 to 1:1) to give (10S,17E)-14-[(2S)-1-{[tert-butyl(dimethyl)silyl]oxy}propan-2-yl]-16-ethoxy-8,10,12-trimethyl-2-(oxan-2-yl)-2,10,11,12,13,14-hexahydro-8H-5,3-(azenometheno)tripyrazolo|3,4-f:3′,4′-j:4″,3″-n][1,4]oxazacyclopentadecine (280 mg, 405 mol, 49% yield) as a white solid. LCMS: (M+1:691.3).

Step 10. To a solution of (10S,17E)-14-[(2S)-1-{[tert-butyl(dimethyl)silyl]oxy}propan-2-yl]-16-ethoxy-8,10,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (200 mg, 289 mol, 1 eq) in DCM (1 mL) was added TFA (30.7 g, 269 mmol, 930 eq). The mixture was stirred at 25° C. for 2 h. On completion, the mixture was concentrated to give a residue. The residue was purified by column chromatography (SiO₂, DCM/MeOH=30:1 to 15:1) to give Ex. 19 (33.5 mg, 68.1 mol, 23% yield) as a white solid. LCMS: (M+1:493.1). ¹H NMR for Ex. 19 can be found in the below NMR table.

### Preparation of 2-[(10R,17E)-6,8,10,16-tetramethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 20) and 2-[(10S,17E)-6,8,10,16-tetramethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 21)

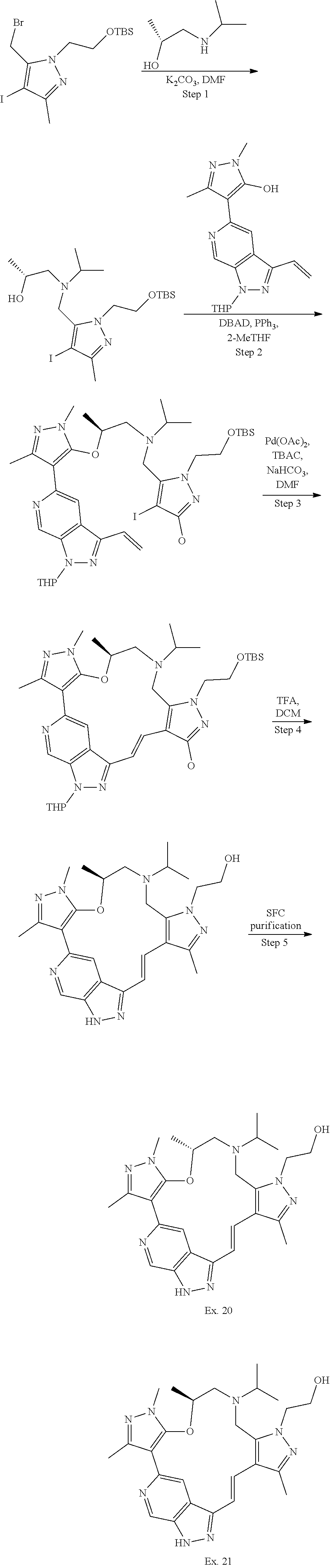

Step 1. To a solution of 2-[5-(bromomethyl)-4-iodo-3-methyl-pyrazol-1-yl]ethoxy-tert-butyl-dimethyl-silane, which was prepared according to the method described in Ex. 13 and 14, (2.30 g, 5.01 mmol, 1.00 eq), commercially available (2R)-1-(isopropylamino)propan-2-ol (1.17 g, 10.0 mmol, 2.00 eq) in DMF (23 mL) was added K₂CO₃ (1.38 g, 10.0 mmol, 2.00 eq). The mixture was stirred at 80° C. for 1 hr. On completion, the reaction mixture was partitioned between ethyl acetate (40 mL×3) and water (150 mL). The combined organic phase was washed with water (50 mL×2) and dried over anhydrous sodium sulfate, filtered and the filtrate was concentrated to give a residue. The residue was purified by column chromatography (SiO₂, Petroleum ether/Ethyl acetate=1/0 to 60/40) to give (2R)-1-[[2-[2-[tert-butyl(dimethyl)silyl]oxyethyl]-4-iodo-5-methyl-pyrazol-3-yl]methyl-isopropylamino]propan-2-ol (1.90 g, 3.83 mmol, 77% yield) as a yellow oil. LCMS: (M+1=496.1).

Step 2. A mixture of (2R)-1-[[2-[2-[tert-butyl(dimethyl)silyl]oxyethyl]-4-iodo-5-methyl-pyrazol-3-yl]methyl-isopropyl-amino]propan-2-ol (964 mg, 1.94 mmol, 1.00 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (858 mg, 2.53 mmol, 1.30 eq), PPh₃ (1.12 g, 4.28 mmol, 2.20 eq), DBAD (985 mg, 4.28 mmol, 2.20 eq) was degassed and purged with N₂ for 3 times, and then to the mixture added 2-MeTHF (30 mL) and stirred at 25° C. for 2 h under N₂ atmosphere. On completion, the mixture was concentrated under reduced pressure to give a residue. The residue was purified by column chromatography (SiO₂, Petroleum ether/Tetrahydrofuran=2/1 to 1/1) to give (2S)-N-[[2-[2-[tert-butyl(dimethyl)silyl]oxyethyl]-4-iodo-5-methyl-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-isopropyl-propan-1-amine (670 mg, 820 μmol, 42% yield) as a colorless solid. LCMS: (M+1=817.5).

Step 3. A mixture of (2S)-N-[[2-[2-[tert-butyl(dimethyl)silyl]oxyethyl]-4-iodo-5-methyl-pyrazol-3-yl]methyl]-2-[2,5-dimethyl-4-(1-tetrahydropyran-2-yl-3-vinyl-pyrazolo [3,4-c]pyridin-5-yl)pyrazol-3-yl]oxy-N-isopropyl-propan-1-amine (330 mg, 404 μmol, 1.00 eq), TBAC (112 mg, 404 μmol, 1.00 eq), NaHCO₃ (84.8 mg, 1.01 mmol, 2.50 eq) and Pd(OAc)₂ (18.1 mg, 80.8 μmol, 0.20 eq) in DMF (3 mL) was degassed and purged with N₂ for 3 times, and then the mixture was stirred at 130° C. for 2 h under N₂ atmosphere. On completion, the reaction mixture was partitioned between ethyl acetate (30 mL×3) and water (100 mL). The combined organic phase was dried over anhydrous sodium sulfate, filtered, and concentrated to give a residue. The residue was purified by column chromatography (SiO₂, Petroleum ether/Tetrahydrofuran=2/1 to 1/1) to give (10S,17E)-14-(2-{[tert-butyl(dimethyl)silyl]oxy}ethyl)-6,8,10,16-tetramethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (120 mg, 174 μmol, 43% yield) as a brown solid. LCMS: (M+1=689.6).

Step 4. To a solution of (10S,17E)-14-(2-{[tert-butyl(dimethyl)silyl]oxy}ethyl)-6,8,10,16-tetramethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (110 mg, 160 μmol, 1.00 eq) in DCM (0.5 mL) was added TFA (0.5 mL). The mixture was stirred at 25° C. for 3 hr. On completion, the mixture was concentrated under reduced pressure to give a residue. The residue was purified by Prep-HPLC purification (column: Xtimate C18 150*40 mm*10 um; mobile phase: [water(TFA)-ACN]; gradient:0%-28% B over 10 min) to give 2-[(10S,17E)-6,8,10,16-tetramethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol and 2-[(10R,17E)-6,8,10,16-tetramethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (50.0 mg, 102 μmol, 64% yield) as a yellow solid.

Step 5. SFC purification: The product obtained above was separated by SFC (condition: column: Phenomenex-Cellulose-2 (250 mm*30 mm, 10 um); mobile phase: [CO₂-MeOH(0.1% NH₃H₂O)]; B %:40%, isocratic elution mode) to give Ex. 20 (7.38 mg, 14.53 mol, 13.85% yield, 96.6% purity) as an orange solid and Ex. 21 (28.43 mg, 57.60 μmol, 54.90% yield, 99.4% purity) as an orange solid.

Ex. 20: LCMS: (M+1=491.1). ¹H NMR for Ex. 20 can be found in the below NMR table.

Ex. 21: LCMS: (M+1=491.2). ¹H NMR for Ex. 21 can be found in the below NMR table.

### Preparation of (2S)-2-[(10R,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 24) and (2S)-2-[(11S,17E)-16-ethoxy-12-ethyl-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 25)

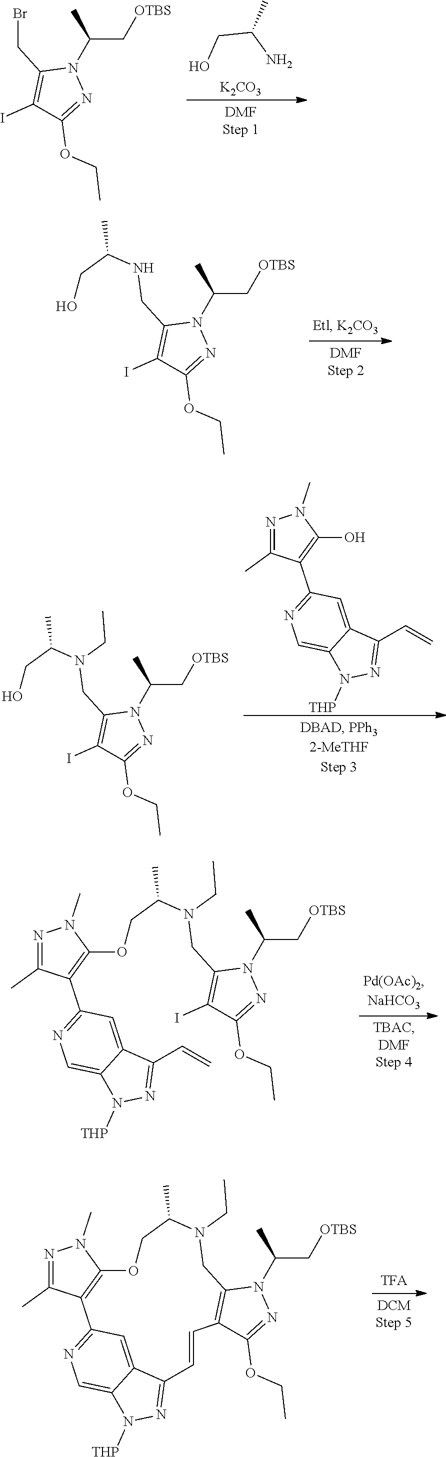

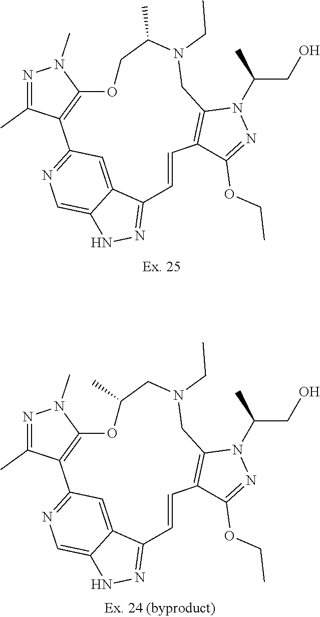 [SMILES: CCOc1nn([C@@H](C)CO)c2c1/C=C/c1n[nH]c3cnc(cc13)-c1c(C)nn(C)c1OC[C@H](C)N(CC)C2 ; CCOc1nn([C@@H](C)CO)c2c1/C=C/c1n[nH]c3cnc(cc13)-c1c(C)nn(C)c1O[C@H](C)CN(CC)C2]

Step 1. To a solution of [(2S)-2-[5-(bromomethyl)-3-ethoxy-4-iodo-pyrazol-1-yl]propoxyl-tert-butyl-dimethyl-silane, which was prepared according to the method described in Ex. 19, (960 mg, 1.91 mmol, 1 eq) in DMF (10 mL) was added K₂CO₃ (791 mg, 5.72 mmol, 3 eq) and commercially available (2S)-2-aminopropan-1-ol (430 mg, 5.72 mmol, 3 eq). The mixture was stirred at 80° C. for 2 hr. On completion, the yellow solution of (S)-2-(((1-((S)-1-((tert-butyldimethylsilyl)oxy)propan-2-yl)-3-ethoxy-4-iodo-1H-pyrazol-5-yl)methyl)amino)propan-1-ol (theoretical quantity: 948 mg, in DMF) was used for the next step directly. LCMS: (M+1=498.1).

Step 2. To the above DMF solution of (S)-2-(((1-((S)-1-((tert-butyldimethylsilyl)oxy)propan-2-yl)-3-ethoxy-4-iodo-1H-pyrazol-5-yl)methyl)amino)propan-1-ol (theoretical quantity from Step 1, 948 mg) was added EtI (2.65 g, 17.0 mmol, 9 eq). The mixture was stirred at 80° C. for 2 hr. On completion, the reaction mixture was diluted with H₂O (100 mL) and extracted with EA (50 mL×3). The combined organic layers were dried over Na₂SO₄, filtered, and concentrated under reduced pressure to give a residue. The residue was purified by column chromatography (SiO₂, Petroleum ether/Ethyl acetate=1/0 to 0/1) to give (S)-2-(((1-((S)-1-((tert-butyldimethylsilyl)oxy)propan-2-yl)-3-ethoxy-4-iodo-1H-pyrazol-5-yl)methyl)(ethyl)amino)propan-1-ol (600 mg, 60% yield) as yellow oil. LCMS: (M+1=526.1).

Step 3. A mixture of (S)-2-(((1-((S)-1-((tert-butyldimethylsilyl)oxy)propan-2-yl)-3-ethoxy-4-iodo-1H-pyrazol-5-yl)methyl)(ethyl)amino)propan-1-ol (550 mg, 1.05 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (462 mg, 1.36 mmol, 1.3 eq), PPh₃ (604 mg, 2.30 mmol, 2.2 eq), and DBAD (530 mg, 2.30 mmol, 2.2 eq) in 2-MeTHF (6 mL) was degassed and purged with N₂ for 3 times, and then stirred at 25° C. for 2 h under N₂ atmosphere. On completion, the reaction mixture was concentrated under reduced pressure to remove solvent. The residue was purified by column chromatography (SiO₂, Petroleum ether/Ethyl acetate=1/0 to 0/1) to give (2S)-N-((1-((S)-1-((tert-butyldimethylsilyl)oxy)propan-2-yl)-3-ethoxy-4-iodo-1H-pyrazol-5-yl)methyl)-1-((1,3-dimethyl-4-(1-(tetrahydro-2H-pyran-2-yl)-3-vinyl-1H-pyrazolo[3,4-c]pyridin-5-yl)-1H-pyrazol-5-yl)oxy)-N-ethylpropan-2-amine (510 mg, 58% yield) as yellow oil. LCMS: (M+1=847.4).

Step 4. A mixture of the product from Step 3 (460 mg, 0.543 mmol, 1 eq), diacetoxypalladium (24.4 mg, 0.109 mmol, 0.2 eq), tetrabutylammonium chloride (151 mg, 0.543 mmol, 1 eq) and NaHCO₃ (114 mg, 1.36 mmol, 2.5 eq) in DMF (5 mL) was degassed and purged with N₂ for 3 times, and then stirred at 140° C. for 1 h under N₂ atmosphere. On completion, the reaction mixture was diluted with H₂O (30 mL) and extracted with EA (15 mL×3). The combined organic layers were dried over Na₂SO₄, filtered, and concentrated under reduced pressure. The residue was purified by column chromatography (SiO₂, Petroleum ether/Ethyl acetate=1/0 to 0/1) to give (11S,17E)-14-[(2S)-1-{[tert-butyl(dimethyl)silyl]oxy}propan-2-yl]-16-ethoxy-12-ethyl-6,8,11-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (294 mg, 75% yield) as yellow oil. ¹H NMR (400 MHz, CDCl₃) δ=9.08 (d, J=4.4 Hz, 1H), 8.48-8.42 (m, 1H), 7.42-7.35 (m, 2H), 5.79-5.72 (m, 1H), 4.65-4.51 (m, 1H), 4.50-4.42 (m, 1H), 4.39-4.31 (m, 2H), 4.11-4.04 (m, 1H), 3.83-3.78 (m, 2H), 3.75-3.72 (m, 3H), 3.71-3.67 (m, 1H), 3.38-3.26 (m, 1H), 2.96 (s, 1H), 2.90-2.88 (m, 1H), 2.86-2.75 (m, 1H), 2.71-2.66 (m, 3H), 2.61-2.49 (m, 1H), 2.45-2.36 (m, 1H), 2.19-2.10 (m, 2H), 1.83-1.73 (m, 2H), 1.70-1.59 (m, 2H), 1.50-1.45 (m, 3H), 1.41-1.37 (m, 3H), 1.35-1.31 (m, 3H), 1.16-1.10 (m, 3H), 0.79-0.76 (m, 9H), −0.04-0.06 (m, 3H), −0.15-0.18 (m, 3H). LCMS: (M+1=719.4).

Step 5. To a solution of the product from Step 4 (260 mg, 0.362 mmol, 1 eq) in DCM (1 mL) was added TFA (3.22 g, 28.2 mmol, 2.10 mL, 78.1 eq). The mixture was stirred at 25° C. for 2 hr. On completion, the reaction mixture was concentrated under reduced pressure to remove solvent. The residue was purified by prep-HPLC (column: Welch Xtimate C18 150*25 mm*5 um; mobile phase: [water(FA)-ACN]; gradient:17%-47% B) and prep-HPLC (column: Phenomenex luna C18 150*25 mm*10 um; mobile phase: [water(FA)-ACN]; gradient:10%-40% B over 8 min) to give Ex. 25 (34.36 mg, 17.34% yield) as yellow solid, and a byproduct Ex. 24 (41.0 mg, 21.56% yield) as yellow solid. ¹H NMR for Ex. 24 and Ex. 25 are in the NMR table below.

### Preparation of 2-[(8aR,9S,19E)-1-ethoxy-9,11,13-trimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol (Ex. 32) and 2-[(8aR,9R,19E)-1-ethoxy-9,11,13-trimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol (Ex. 33)

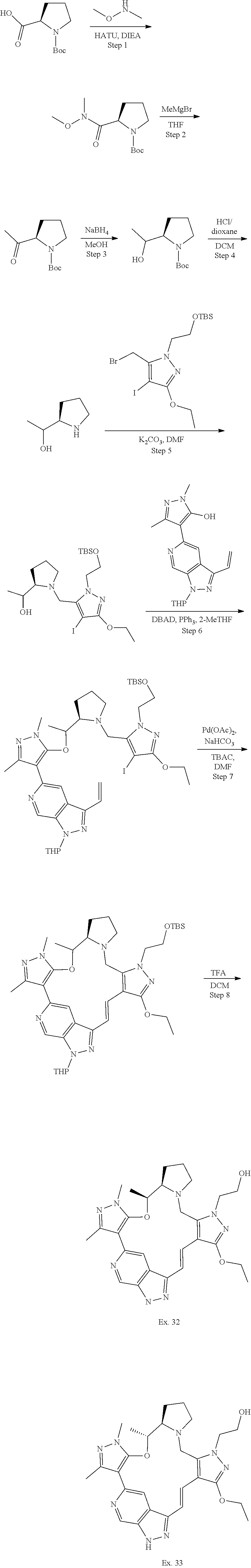

Step 1. To a solution of commercially available (2R)-1-tert-butoxycarbonylpyrrolidine-2-carboxylic acid (20.0 g, 92.9 mmol, 1 eq), commercially available N-methoxymethanamine (6.24 g, 102 mmol, 1.10 eq), DIEA (24.0 g, 185 mmol, 2.00 eq), HATU (38.8 g, 102 mmol, 1.10 eq) in DCM (200 mL). The mixture was stirred at 25° C. for 1 hr. On completion, the mixture was diluted with water (300 mL) and extracted with DCM (60 mL×3). The combined organic phase was washed with water (200 mL×2) and dried over Na₂SO₄, filtered and the filtrate was concentrated to give a residue. The residue was purified by column chromatography (SiO₂, DCM/MeOH=1:0 to 90:10) to give tert-butyl (R)-2-(methoxy(methyl)carbamoyl)pyrrolidine-1-carboxylate(11.0 g, 42.5 mmol, 46% yield) as a yellow oil. LCMS: (M+1-100:159.1).

Step 2. To a solution of tert-butyl (2R)-2-[methoxy(methyl)carbamoyl]pyrrolidine-1-carboxylate (10.0 g, 38.7 mmol, 1.00 eq) in THE (100 mL) was added bromo(methyl)magnesium (3 M, 38.7 mL, 3.00 eq) at −78° C. under atmosphere. The mixture was stirred at −78° C. for 2 hr. On completion, the reaction mixture was quenched with sat. NH₄Cl (200 mL) at 0° C., and then diluted with H₂O (150 mL) and extracted with EA (60 mL×3). The combined organic layers were dried over Na₂SO₄, filtered and concentrated under reduced pressure to give a residue. The residue was purified by column chromatography (SiO₂, Petroleum ether/Ethyl acetate=1:0 to 1:1) to give tert-butyl (R)-2-acetylpyrrolidine-1-carboxylate (7.00 g, 32.8 mmol, 85% yield) as a yellow oil. ¹H NMR (400 MHz, CDCl₃) δ=4.37-4.13 (m, 1H), 3.60-3.38 (m, 2H), 2.31-2.00 (m, 4H), 1.92-1.76 (m, 3H), 1.52-1.37 (m, 9H).

Step 3. To a solution of tert-butyl (2R)-2-acetylpyrrolidine-1-carboxylate (6.50 g, 30.4 mmol, 1.00 eq) in MeOH (65 mL) was added NaBH₄ (3.46 g, 91.4 mmol, 3.00 eq) at 0° C. The mixture was stirred at 0° C. for 2 hr. On completion, the mixture was concentrated to give a residue. The residue was purified by column chromatography (SiO₂, Petroleum ether/Ethyl acetate=1:0 to 1:1) to give tert-butyl (2R)-2-(1-hydroxyethyl)pyrrolidine-1-carboxylate (5.60 g, 26.0 mmol, 85% yield) as a yellow oil. ¹H NMR (400 MHz, CDCl₃) δ=4.01-3.81 (m, 1H), 3.77-3.45 (m, 2H), 3.31-3.20 (m, 1H), 2.05-1.64 (m, 4H), 1.46 (s, 9H), 1.16-1.03 (m, 3H). LCMS: (M+1-100:116.2).

Step 4. To a solution of tert-butyl (2R)-2-(1-hydroxyethyl)pyrrolidine-1-carboxylate (1.00 g, 4.64 mmol, 1.00 eq) in DCM (10 mL) was added HCl/dioxane (4 M, 2.00 mL, 1.72 eq). The mixture was stirred at 25° C. for 2 hr. On completion, the mixture was concentrated to give 1-((R)-pyrrolidin-2-yl)ethan-1-ol (700 mg, crude) as a white oil.

Step 5. To a solution of 1-((R)-pyrrolidin-2-yl)ethan-1-ol (284 mg, 1.87 mmol, 1 eq, HCl) and 2-[5-(bromomethyl)-3-ethoxy-4-iodo-pyrazol-1-yl]ethoxy-tert-butyl-dimethyl-silane, prepared according to the method described in Ex. 1, (1.01 g, 2.06 mmol, 1.1 eq) in DMF (10 mL) was added K₂CO₃ (777 mg, 5.62 mmol, 3 eq). The mixture was stirred at 80° C. for 1 h. On completion, the mixture was quenched with water (20 mL) and extracted with ethyl acetate (25 mL×3). The combined organic phase was dried over anhydrous sodium sulfate, filtered and concentrated to give a residue. The residue was purified by column chromatography (SiO₂, Petroleum ether/Ethyl acetate=1:0 to 0:1) to give 1-[(2R)-1-[[2-[2-[tert-butyl(dimethyl)silyl]oxyethyl]-5-ethoxy-4-iodo-pyrazol-3-yl]methyl]pyrrolidin-2-yl]ethanol (1.07 g, 1.84 mmol, 98% yield, 90% purity) as off-white oil. LCMS: (M+1=524.0).

Step 6. A mixture of 1-[(2R)-1-[[2-[2-[tert-butyl(dimethyl)silyl]oxyethyl]-5-ethoxy-4-iodo-pyrazol-3-yl]methyl]pyrrolidin-2-yl]ethanol (970 mg, 1.85 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (1.01 g, 2.96 mmol, 1.6 eq), DBAD (939 mg, 4.08 mmol, 2.2 eq), PPh₃ (1.07 g, 4.08 mmol, 2.2 eq) in 2-MeTHF (10 mL) was degassed and purged with N₂ for 3 times, and then the mixture was stirred at 25° C. for 2 h under N₂ atmosphere. On completion, the mixture was quenched with water (20 mL) and extracted with ethyl acetate (25 mL×3). The combined organic phase was dried over anhydrous sodium sulfate, filtered and concentrated to give a residue. The residue was purified by column chromatography (SiO₂, Petroleum ether/Ethyl acetate=1:0 to 0:1) to give compound tert-butyl-[2-[5-[[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]methyl]-3-ethoxy-4-iodo-pyrazol-1-yl]ethoxy]-dimethyl-silane (1.00 g, 1.18 mmol, 64% yield) as yellow oil. ¹H NMR (400 MHz, CDCl₃-d) 6=9.13 (s, 1H), 7.97-7.81 (m, 1H), 7.10-6.93 (m, 1H), 6.18-6.03 (m, 1H), 5.87-5.71 (m, 1H), 5.63-5.49 (m, 1H), 4.27-4.19 (m, 2H), 4.06 (br s, 1H), 3.79-3.73 (m, 2H), 3.68 (br d, J=17.2 Hz, 4H), 3.54-3.44 (m, 1H), 2.84-2.72 (m, 1H), 2.58-2.47 (m, 1H), 2.43-2.33 (m, 4H), 2.21-2.06 (m, 2H), 1.89-1.62 (m, 8H), 1.46-1.34 (m, 5H), 1.12-1.00 (m, 3H), 0.91-0.83 (m, 2H), 0.81-0.71 (m, 9H), −0.07-0.20 (m, 6H). LCMS: (M+1=845.5).

Step 7. A mixture of tert-butyl-[2-[5-[[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]methyl]-3-ethoxy-4-iodo-pyrazol-1-yl]ethoxy]-dimethyl-silane (420 mg, 497 mol, 1 eq), diacetoxypalladium (22.3 mg, 99.4 mol, 0.2 eq), TBAC (138 mg, 497 mol, 1 eq) and NaHCO₃ (104 mg, 1.24 mmol, 2.5 eq) in DMF (10 mL) was degassed and purged with N₂ for 3 times, and then the mixture was stirred at 120° C. for 1 hr under N₂ atmosphere. On completion, the mixture was quenched with water (20 mL) and extracted with ethyl acetate (25 mL×3). The combined organic phase was dried over anhydrous sodium sulfate, filtered, and concentrated to give a residue. The residue was purified by column chromatography (SiO₂, Petroleum ether/THF=1:0 to 0:1) to give (8aR,19E)-3-(2-{[tert-butyl(dimethyl)silyl]oxy}ethyl)-1-ethoxy-9,11,13-trimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecine (327 mg, 456 μmol, 92% yield) as yellow solid. LCMS: (M+1=717.2).

Step 8. To a solution of (8aR,19E)-3-(2-{[tert-butyl(dimethyl)silyl]oxy}ethyl)-1-ethoxy-9,11,13-trimethyl-17-(oxan-2-yl)-3,4,7,8,8a,9,11,17-octahydro-6H-14,16-(azenometheno)tripyrazolo[3,4 f:3′,4′-j:4″,3″-n]pyrrolo[2,1-c][1,4]oxazacyclopentadecine (313 mg, 437 mol, 1 eq) in DCM (3 mL) was added TFA (40.4 mmol, 3 mL, 92.5 eq). The mixture was stirred at 25° C. for 1 h. On completion, the mixture was filtrated. The filtrate was purified by prep-HPLC(column: Phenomenex Luna C18 150*30 mm*5 um; mobile phase: [water(FA)-ACN]; gradient:10%-40% B over 10 min) to give Ex. 32 (31.73 mg, 55.76 mol, 12.77% yield, 99.22% purity, FA) as yellow solid and Ex. 33 (34.49 mg, 66.50 μmol, 15.23% yield) as yellow solid.

Ex. 32: LCMS: (M+1=519.3). ¹H NMR for Ex. 32 is shown in the below NMR table.

Ex. 33: LCMS: (M+1=519.2). ¹H NMR for Ex. 33 is shown in the below NMR table.

### Preparation of 2-[(17E)-16-ethoxy-6,8,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 68)

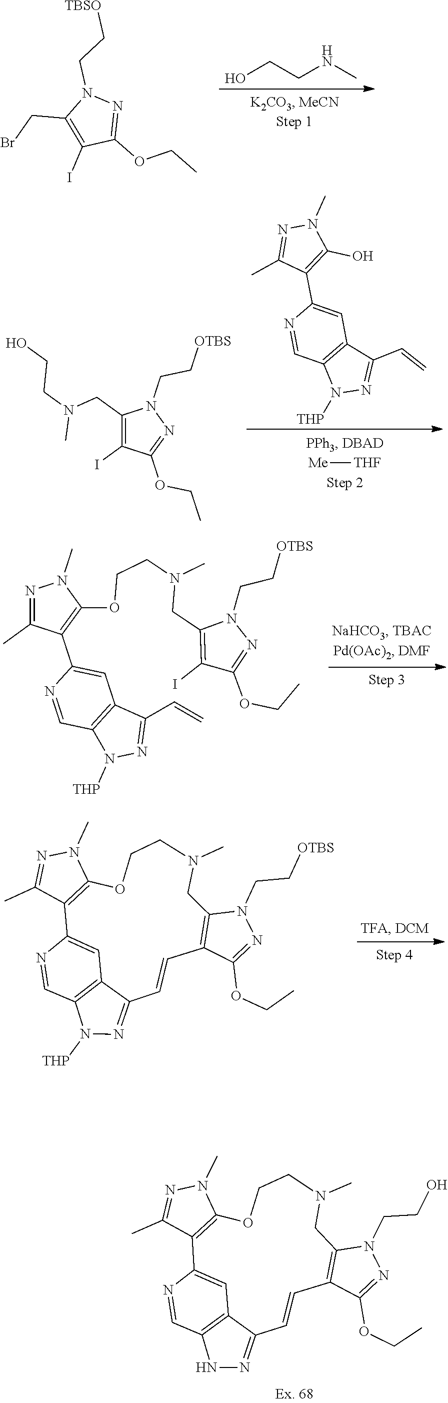

Step 1. To 2-[5-(bromomethyl)-3-ethoxy-4-iodo-pyrazol-1-yl]ethoxy-tert-butyl-dimethyl-silane, prepared according to the method described in Ex. 1, (508 mg, 1.04 mmol) in Acetonitrile (5.09 mL) was added potassium carbonate (430.51 mg, 3.11 mmol) followed by commercially available 2-(methylamino)ethanol (93.58 mg, 1.25 mmol, 99.66 μL). The reaction mixture was stirred at 22° C. for 18 hr. The solution was diluted in DCM (5 mL) and cooled in an ice bath. The solids were filtered through a celite pad and washed thoroughly with DCM. The filtrate was concentrated via rotary evaporation and the residue was purified by flash column chromatography (automated system, 12 g silica, 0-10% Methanol in DCM) to provide 2-[[2-[2-[tert-butyl(dimethyl)silyl]oxyethyl]-5-ethoxy-4-iodo-pyrazol-3-yl]methyl-methyl-amino]ethanol (404 mg, 835.65 μmol, 80.48% yield) as a colorless oil.

Step 2. To the mixture of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (75 mg, 220.98 μmol), 2-[[2-[2-[tert-butyl(dimethyl)silyl]oxyethyl]-5-ethoxy-4-iodo-pyrazol-3-yl]methyl-methyl-amino]ethanol (106.84 mg, 220.98 μmol), and Triphenylphosphine (86.94 mg, 331.48 mol) in dry Methyl-THF (441.97 μL) at 0° C. was added DBAD (81.41 mg, 353.57 mol). The mixture was stirred at 22° C. for 18 hr. Reaction was diluted with DCM and water (4 mL) and the layers were separated. The aqueous layer was extracted again with DCM (2×3 mL). The combined organic layer was washed with brine and dried over sodium sulfate. Flash column chromatography (automated system, 12 g silica, 0-100% EA in Hexanes) provided N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-ethanamine (114 mg, 141.64 mol, 64.10% yield).

Step 3. To the solution of N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-ethanamine (114 mg, 141.64 mol) in DMF (1.40 mL), was added sodium bicarbonate (36 mg, 424.9 μmol) and TBAC (43.3 mg, 155.8 μmol). The mixture was stirred as argon was bubbled through and palladium acetate (6.4 mg, 28.3 μmol) was then added. The vessel was sealed and heated to 140° C. for 1.5 hr. Reaction was diluted with DCM and water (4 mL) and the layers were separated. The aqueous layer was extracted again with DCM (2×4 mL). The combined organic layer was washed with brine and dried over sodium sulfate. Flash column chromatography (automated system, 12 g silica, 0-100% EA in Hexanes) provided crude (17E)-14-(2-{[tert-butyl(dimethyl)silyl]oxy}ethyl)-16-ethoxy-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (40 mg, 59.09 mol, 41.72% yield) as a brown oil.

Step 4. To the mixture of crude (17E)-14-(2-{[tert-butyl(dimethyl)silyl]oxy}ethyl)-16-ethoxy-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (40 mg, 59.09 μmol) in DCM (2 mL) was added TFA (2.61 mmol, 0.2 mL). The mixture was stirred at 22° C. for 18 hr. On completion, the reaction mixture was concentrated under reduced pressure, and the residue was diluted in DCM (1 mL) and 0.1 mL of TEA was added. Flash column chromatography (automated system, 12 g silica, 0-10% MeOH in DCM) provided Ex. 68 (2.60 mg, 5.38 mol, 9.11% yield, 99.03% purity). LC/MS: (M+1: 479.3). ¹H NMR for Ex. 68 is shown in the below NMR table.

### Preparation of 2-[(17E)-16-ethoxy-6,8,12-trimethyl-2,6,10,11,12,13-hexahydro-14H-5,3-(azenometheno)tripyrazolo[3,4-f:3′,4′-j:3″,4″-n][1,4]oxazacyclopentadecin-14-yl]ethan-1-ol (Ex. 69)

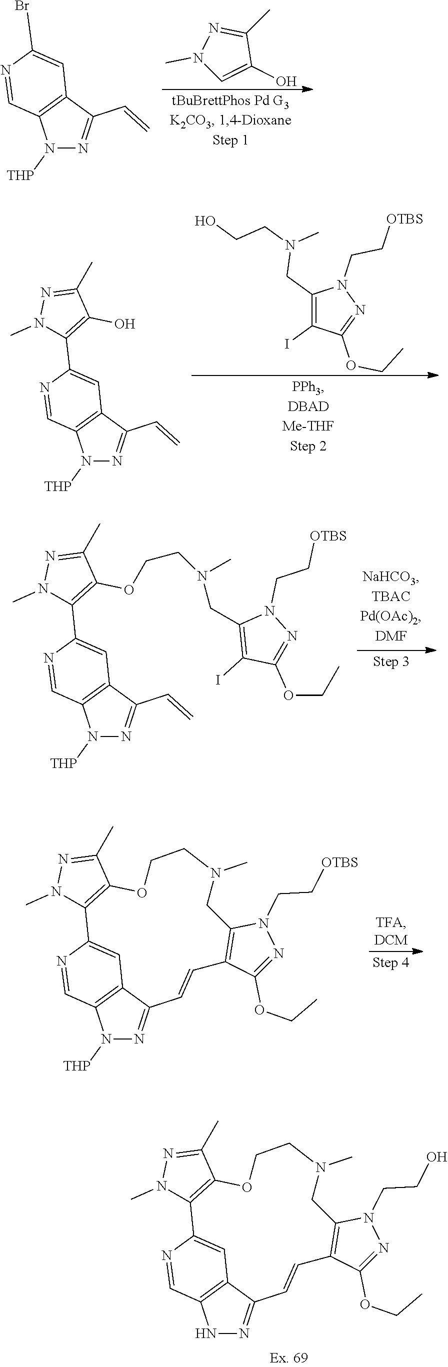

Step 1. To 5-bromo-1-tetrahydropyran-2-yl-3-vinyl-pyrazolo[3,4-c]pyridine, which was prepared according to the method described in Ex. 1, (250 mg, 811.23 mol) in 1,4-Dioxane (4 mL) was added commercially available 1,3-dimethylpyrazol-4-ol (109.2 mg, 973.5 μmol), potassium carbonate (336 mg, 2.43 mmol). Argon was bubbled through and tBuBrettphos Pd G3 (69 mg, 81.12 μmol) was added. The mixture was stirred at 130° C. for 1.5 hr. Reaction was diluted with DCM and water (10 mL) and the layers were separated. The aqueous layer was extracted again with DCM (2×10 mL). The combined organic layer was washed with brine and dried over sodium sulfate. Flash column chromatography (automated system, 12 g silica, 20-80% EA in Hexanes) provided 1,3-dimethyl-5-(1-tetrahydropyran-2-yl-3-vinyl-pyrazolo[3,4-c]pyridin-5-yl)pyrazol-4-ol.

Step 2. To 1,3-dimethyl-5-(1-tetrahydropyran-2-yl-3-vinyl-pyrazolo[3,4-c]pyridin-5-yl)pyrazol-4-ol (10.7 mg, 31.53 mol), 2-[[2-[2-[tert-butyl(dimethyl)silyl]oxyethyl]-5-ethoxy-4-iodo-pyrazol-3-yl]methyl-methyl-amino]ethanol, which was prepared according to the method described in Ex. 68, (23 mg, 47.3 mol), and triphenylphosphine (12.4 mg, 47.3 μmol) in dry Me-THF (0.2 mL) at ° C. was added DBAD (12 mg, 50.4 μmol). The mixture was stirred at 22° C. for 18 hrs. Flash column chromatography (automated system, 12 g silica, 0-100% EA in Hexanes) provided N-[[2-[2-[tert-butyl(dimethyl)silyl]oxyethyl]-5-ethoxy-4-iodo-pyrazol-3-yl]methyl]-2-[1,3-dimethyl-5-(1-tetrahydropyran-2-yl-3-vinyl-pyrazolo[3,4-c]pyridin-5-yl)pyrazol-4-yl]oxy-N-methyl-ethanamine (7.85 mg, 9.75 mol, 30.94% yield).

Step 3. To N-[[2-[2-[tert-butyl(dimethyl)silyl]oxyethyl]-5-ethoxy-4-iodo-pyrazol-3-yl]methyl]-2-[1,3-dimethyl-5-(1-tetrahydropyran-2-yl-3-vinyl-pyrazolo[3,4-c]pyridin-5-yl)pyrazol-4-yl]oxy-N-methyl-ethanamine (7.85 mg, 9.75 μmol) in anhydrous DMF (0.5 mL) was added Sodium bicarbonate (2.46 mg, 29.26 mol, 1.14 μL) and TBAC (2.98 mg, 10.73 mol). The reaction mixture was stirred while Argon was bubbled through, and palladium acetate (2.19 mg, 9.75 mol, 1 μL) was added. Argon was bubbled through for an additional 5 minutes. The vessel was sealed, and the reaction heated to 140° C. for 1.5 hr. Reaction was diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 30-100% EA in Hexanes, injected with 0.5 mL of TEA) provided (17E)-14-(2-{[tert-butyl(dimethyl)silyl]oxy}ethyl)-16-ethoxy-6,8,12-trimethyl-2-(oxan-2-yl)-2,10,11,12,13,14-hexahydro-6H-5,3-(azenometheno)tripyrazolo[3,4-f:3′,4′-j:3″,4″-n][1,4]oxazacyclopentadecine (2.29 mg, 3.38 mol, 34.68% yield).

Step 4. To (17E)-14-(2-{[tert-butyl(dimethyl)silyl]oxy}ethyl)-16-ethoxy-6,8,12-trimethyl-2-(oxan-2-yl)-2,10,11,12,13,14-hexahydro-6H-5,3-(azenometheno)tripyrazolo 3,4-f:3′,4′-j:3″,4″-n][1,4]oxazacyclopentadecine (2.29 mg, 3.38 μmol) in DCM (0.5 mL) was added TFA (1.31 mmol, 0.1 mL). The mixture was stirred at 22° C. for 18 hr. Volatiles were removed under reduced pressure. Then 0.2 mL of TEA was added and the residue was purified flash column chromatography (automated system, 12 g silica, 0-40% MeOH in DCM) provided Ex. 69 (0.8 mg, 1.67 mol, 49.42% yield, LC/MS: (M+H: 479.3). ¹H NMR for Ex. 69 is shown in the below NMR table.

### Preparation of (10S,17E)-6,8,10,14,16,20-hexamethyl-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (Ex. 106) and (11R,17E)-6,8,11,14,16,20-hexamethyl-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (Ex. 108)

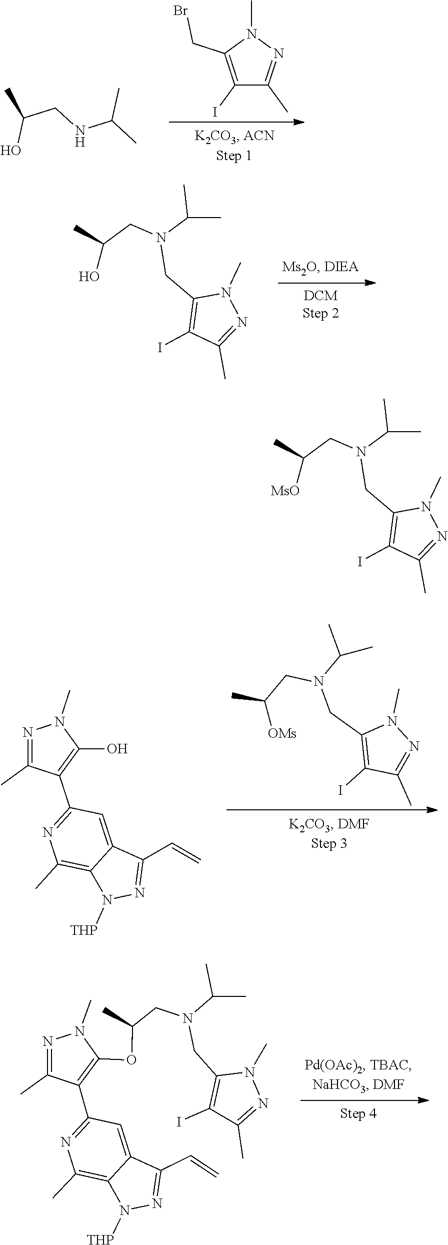

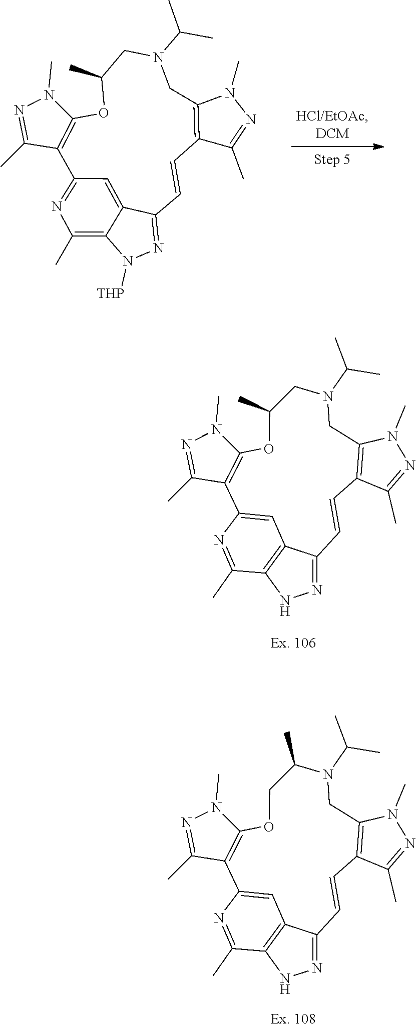

Step 1. To a solution of commercially available (2S)-1-(isopropylamino)propan-2-ol (600 mg, 5.12 mmol, 1.5 eq) in ACN (6 mL) was added commercially available 5-(bromomethyl)-4-iodo-1,3-dimethyl-pyrazole (,1.08 g, 3.41 mmol, 1 eq) and K₂CO₃ (1.42 g, 10.2 mmol, 3 eq). The mixture was stirred at 80° C. for 2 hrs. On completion, the reaction mixture was filtered to remove K₂CO₃ and then was concentrated in vacuum. The residue was purified by column chromatography (SiO₂, Petroleum ether/Ethyl acetate=1:0 to 1:1) to give (2S)-1-[(4-iodo-2,5-dimethyl-pyrazol-3-yl)methyl-isopropyl-amino]propan-2-ol (1.15 g, 3.21 mmol, 94% yield) as an orange oil. ¹H NMR (400 MHz, DMSO-d₆) δ=4.20 (d, J=4.0 Hz, 1H), 3.81 (s, 3H), 3.67-3.61 (m, 1H), 3.55 (d, J=13.6 Hz, 1H), 2.77-2.61 (m, 2H), 2.30-2.24 (m, 1H), 2.20-2.14 (m, 1H), 2.07 (s, 3H), 0.97 (d, J=3.6 Hz, 3H), 0.96 (d, J=3.2 Hz, 3H), 0.93 (d, J=6.0 Hz, 3H). LCMS: (M+1: 351.9).

Step 2. To a solution of (2S)-1-[(4-iodo-2,5-dimethyl-pyrazol-3-yl)methyl-isopropyl-amino]propan-2-ol (1.05 g, 2.99 mmol, 1 eq) and DIEA (1.16 g, 8.97 mmol, 1.56 mL, 3 eq) in DCM (10 mL) was added methylsulfonyl methanesulfonate (1.04 g, 5.98 mmol, 2 eq) at 0° C. The mixture was stirred at 25° C. for 0.5 hour. On completion, the reaction mixture diluted with H₂O (50 mL) and extracted with DCM (15 mL×3). The combined organic phase was dried over anhydrous sodium sulfate, filtered and concentrated under reduced pressure to give [(1S)-2-[(4-iodo-2,5-dimethyl-pyrazol-3-yl)methyl-isopropyl-amino]-1-methyl-ethyl]methanesulfonate (1.37 g, crude) as an orange oil. LCMS: (M+1: 429.9).

Step 3. To a solution of [(1S)-2-[(4-iodo-2,5-dimethyl-pyrazol-3-yl)methyl-isopropyl-amino]-1-methyl-ethyl]methanesulfonate (1.37 g, 3.19 mmol, 1 eq) in DMF (14 mL) was added 2,5-dimethyl-4-(7-methyl-1-tetrahydropyran-2-yl-3-vinyl-pyrazolo[3,4-c]pyridin-5-yl)pyrazol-3-ol, which was prepared according to the method described in Ex. 47, (1.13 g, 3.19 mmol, 1 eq) and K₂CO₃ (1.32 g, 9.57 mmol, 3 eq). The mixture was stirred at 80° C. for 0.5 hour. On completion, the reaction mixture was diluted with H₂O (150 mL) and extracted with EA (100 mL×3). The combined organic layers were washed with H₂O (100 mL), dried over Na₂SO₄, filtered, and concentrated under reduced pressure. The residue was purified by column chromatography (SiO₂, Petroleum ether/Ethyl acetate=1:0 to 2:1) to give (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-[(4-iodo-2,5-dimethyl-pyrazol-3-yl)methyl]-N-isopropyl-propan-1-amine (1.78 g, 2.51 mmol, 78% yield, 97% purity) as an orange oil. ¹H NMR (400 MHz, DMSO-d₆) δ=7.58 (s, 1H), 7.22 (d, J=11.6, 17.6 Hz, 1H), 6.06-5.90 (m, 2H), 5.68-5.59 (m, 1H), 4.03 (q, J=7.2 Hz, 2H), 3.93-3.73 (m, 2H), 3.62-3.49 (m, 6H), 2.76-2.70 (m, 3H), 2.27-2.16 (m, 3H), 2.04-1.96 (m, 6H), 1.61 (s, 2H), 1.35 (s, 2H), 1.17 (t, J=7.2 Hz, 3H), 1.07 (d, J=6.4 Hz, 1H), 1.00-0.91 (m, 3H), 0.85 (d, J=6.4, 13.6 Hz, 5H). LCMS: (M+1: 687.1).

Step 4. To a solution of the product from Step 3 (1.20 g, 1.75 mmol, 1 eq) in DMF (12 mL) was added NaHCO₃ (734 mg, 8.74 mmol, 5 eq) and TBAC (971 mg, 3.50 mmol, 2 eq) and Pd(OAc)₂ (58.9 mg, 262 μmol, 0.15 eq) was degassed and purged with N₂ for 3 times. The mixture was stirred at 130° C. for 1 hour under N₂ atmosphere. On completion, the reaction mixture was diluted with H₂O (100 mL) and extracted with EA (100 mL×3). The combined organic phase was dried over anhydrous sodium sulfate, filtered and concentrated under reduced pressure to give (10S,17E)-6,8,10,14,16,20-hexamethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (750 mg, crude) as a black brown oil. LCMS: (M+1: 559.4).

Step 5. To a solution of the product from Step 4 (600 mg, 1.07 mmol, 1 eq) in DCM (6 mL) was added HCl/EtOAc (4.00 M, 3 mL, 11.2 eq). The mixture was stirred at 25° C. for 1 hour. On completion, the reaction was concentrated in vacuum. The residue was purified by prep-HPLC purification (column: Phenomenex luna C₁₈ 150*25 mm*10 um; mobile phase: [water (FA)-ACN]; gradient: 12%-42% B over 11 min) to give Ex. 106 (94.81 mg, 17.63% yield) as a yellow solid and a byproduct Ex. 108 (15.22 mg, 2.87% yield) as an off-white solid. ¹H NMR for Ex. 106 and Ex. 108 are in the below NMR table.

### Preparation of (2S)-1-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol (Ex. 107)

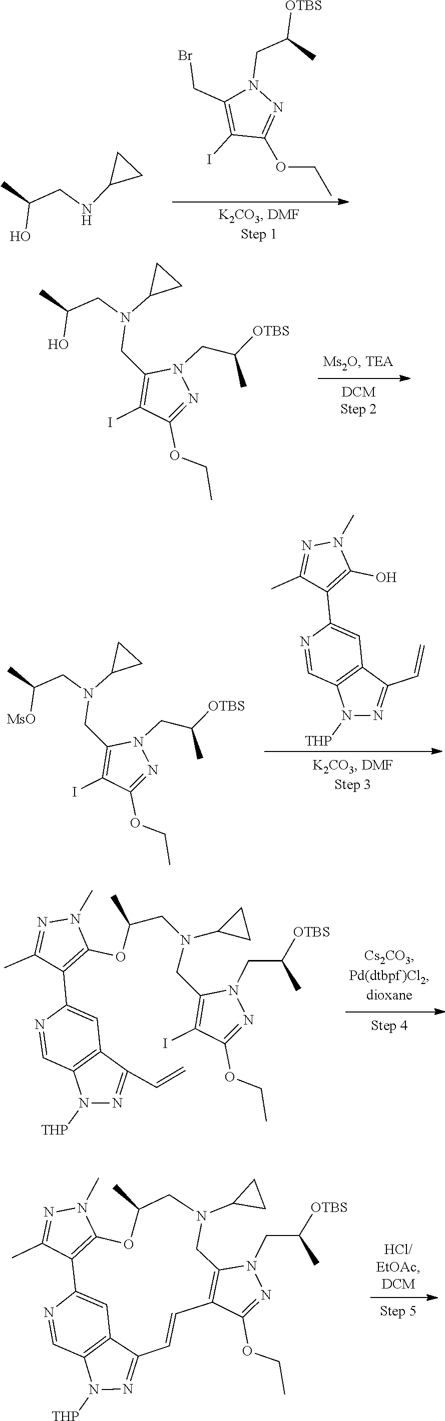

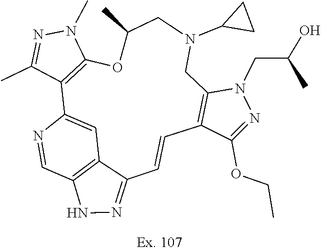 [SMILES: CCOc1nn(C[C@H](C)O)c2c1/C=C/c1n[nH]c3cnc(cc13)-c1c(C)nn(C)c1O[C@@H](C)CN(C1CC1)C2]

Step 1. To a solution of commercially available (2S)-1-(cyclopropylamino)propan-2-ol (140 mg, 1.22 mmol, 1.5 eq.), [(1S)-2-[5-(bromomethyl)-3-ethoxy-4-iodo-pyrazol-1-yl]-1-methyl-ethoxy]-tert-butyl-dimethyl-silane (407 mg, 810 mol, 1 eq), which was prepared according to the method described in Ex. 86, in DMF (5 mL) was added K₂CO₃ (335 mg, 2.43 mmol, 3 eq). The mixture was stirred at 80° C. for 0.5 hours. On completion, the reaction mixture was partitioned between ethyl acetate (10 mL×3) and water (15 mL); and the combined organic phase was dried over anhydrous sodium sulfate, filtered and concentrated. The residue was purified by column chromatography (SiO₂, Petroleum ether/Ethyl acetate=1:0 to 0:1) to give (2S)-1-[[2-[(2S)-2-[tert-butyl(dimethyl)silyl]oxypropyl]-5-ethoxy-4-iodo-pyrazol-3-yl]methyl-cyclopropyl-amino]propan-2-ol (400 mg, 744 μmol, 91% yield) as yellow oil. LCMS: (M+1: 538.1).

Step 2. To a solution of the product from Step 1 (400 mg, 744 mol, 1 eq) in DCM (4 mL) was added TEA (225 mg, 2.23 mmol, 3 eq) and methylsulfonyl methanesulfonate (324 mg, 1.86 mmol, 2.5 eq). The mixture was stirred at 25° C. for 2 hours. On completion, the reaction mixture was partitioned between dichloromethane (5 mL×3) and water (5 mL); and the combined organic phase was dried over anhydrous sodium sulfate, filtered and concentrated to give [(1S)-2-[[2-[(2S)-2-[tert-butyl(dimethyl)silyl]oxypropyl]-5-ethoxy-4-iodo-pyrazol-3-yl]methyl-cyclopropyl-amino]-1-methyl-ethyl]methanesulfonate (400 mg, 87% yield) as a yellow solid. LCMS: (M+1: 616.2).

Step 3. To a solution of the mesylate from Step 2 (240 mg, 389 mol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (132 mg, 389 μmol, 1 eq) in DMF (4 mL) was added K₂CO₃ (161 mg, 1.17 mmol, 3 eq). The mixture was stirred at 80° C. for 0.5 hours. On completion, the reaction mixture was partitioned between ethyl acetate (10 mL×3) and water (12 mL); and the combined organic phase was dried over anhydrous sodium sulfate, filtered and concentrated to give a residue. The residue was purified by column chromatography (SiO₂, Petroleum ether/Ethyl acetate=1:0 to 0:1) to give N-[[2-[(2S)-2-[tert-butyl(dimethyl)silyl]oxypropyl]-5-ethoxy-4-iodo-pyrazol-3-yl]methyl]-N-[(2S)-2-[2,5-dimethyl-4-(1-tetrahydropyran-2-yl-3-vinyl-pyrazolo[3,4-c]pyridin-5-yl)pyrazol-3-yl]oxypropyl]cyclopropanamine (140 mg, 41% yield) as yellow oil. LCMS: (M+1: 859.3).

Step 4. A mixture of the product from Step 3 (60.0 mg, 69.8 μmol, 1 eq), Cs₂CO₃ (68.2 mg, 209 μmol, 3 eq), ditert-butyl(cyclopentyl)phosphane, and dichloropalladium; iron (Pd(dtbpf)Cl₂) (22.7 mg, 34.9 mol, 0.5 eq) in dioxane (1 mL) was degassed and purged with N₂ for 3 times. Then the mixture was stirred at 80° C. for 10 min under N₂ atmosphere. On completion, the mixture was filtered, and the filtrate concentrated. The residue was purified by column chromatography (SiO₂, Petroleum ether/Ethyl acetate=1:0 to 0:1) to give (10S,17E)-14-[(2S)-2-{[tert-butyl(dimethyl)silyl]oxy}propyl]-12-cyclopropyl-16-ethoxy-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (10.0 mg, 19% yield) as yellow oil. LCMS: (M+1: 731.3).

Step 5. To a solution of the product from Step 4 (5.00 mg, 6.84 mol, 1 eq) in DCM (1 mL) was added HCl/EtOAc (4 M, 0.5 mL, 3 eq). The mixture was stirred at 25° C. for 0.5 hours. On completion, the mixture was filtered, and filtrate concentrated to give a residue. The crude product was purified by reversed-phase HPLC(column: Phenomenex luna C18 150*25 mm*10 um; mobile phase: [water(FA)-ACN]; gradient:26%-56% B over 10 min) to give Ex. 107 (0.98 mg, 23% yield) as a yellow solid. ¹H NMR for Ex. 107 is shown in the below NMR table.

### Preparation of 2-[(10S,17E)-6,8,10,16,20-pentamethyl-12-(propan-2-yl)-2,8,10,11,12,13-hexahydro-14H-5,3-(azenometheno)tripyrazolo[3,4-f3′,4′-j:4″,3″-n][1,4]oxazacyclopentadecin-14-yl]ethan-1-ol (Ex. 109)

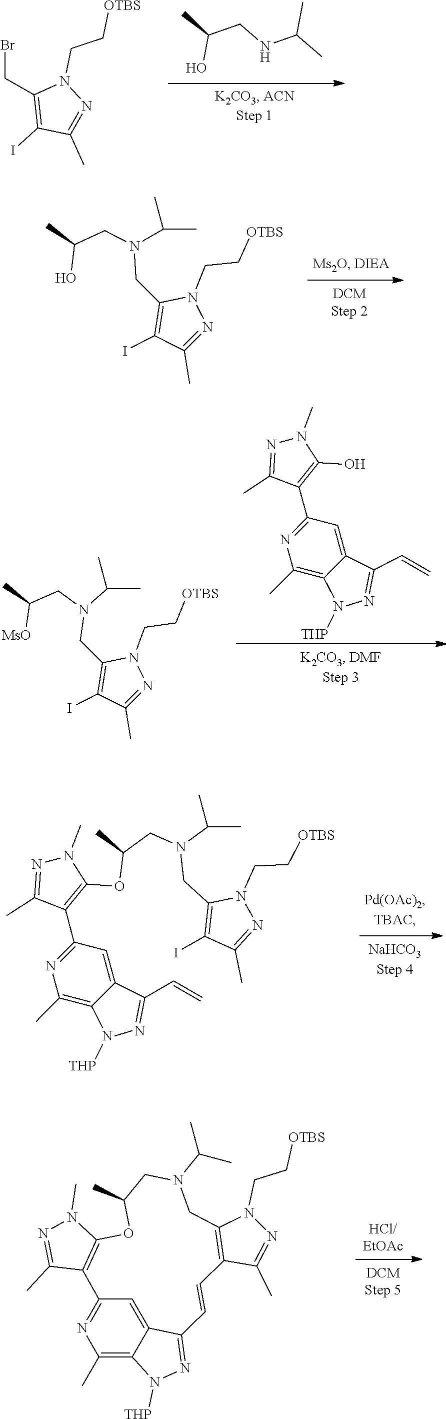

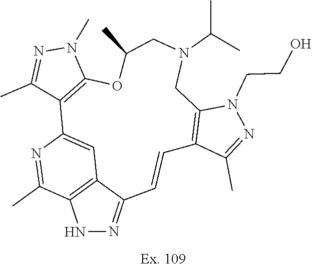

Step 1. The two starting materials were prepared as in Ex. 8 and Ex. 14. And the chemistry was carried out in the same manner as in Ex. 8.

Step 2, Step 3, and Step 4 were carried out in the same manner as in Ex. 108 using 2,5-dimethyl-4-(7-methyl-1-tetrahydropyran-2-yl-3-vinyl-pyrazolo[3,4-c]pyridin-5-yl)pyrazol-3-ol in Step 3 as prepared according to the method described in Ex. 47.

Step 5. To a solution of (10S,17E)-14-(2-{[tert-butyl(dimethyl)silyl]oxy}ethyl)-6,8,10,16,20-pentamethyl-2-(oxan-2-yl)-12-(propan-2-yl)-2,10,11,12,13,14-hexahydro-8H-5,3-(azenometheno)tripyrazolo[3,4-f:3′,4′-j:4″,3″-n][1,4]oxazacyclopentadecine (85.0 mg, 0.121 mmol, 1 eq) in DCM (8 mL) was added HCl/EtOAc (2 M, 4 mL, 66 eq). The mixture was stirred at 25° C. for 1 hr. On completion, the mixture was concentrated in vacuum. The residue was purified by prep-HPLC (column: Welch Ultimate C18 150*25 mm*5 um; mobile phase: [water(FA)-ACN]; gradient:0%-30% B over 10 min) to give Ex. 109 (19.97 mg, 31.09% yield) as a yellow solid. ¹H NMR for Ex. 109 is in the below NMR table.

### Preparation of (2S)-2-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 110) and (2S)-2-[(11R,17E)-12-cyclopropyl-16-ethoxy-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 111)

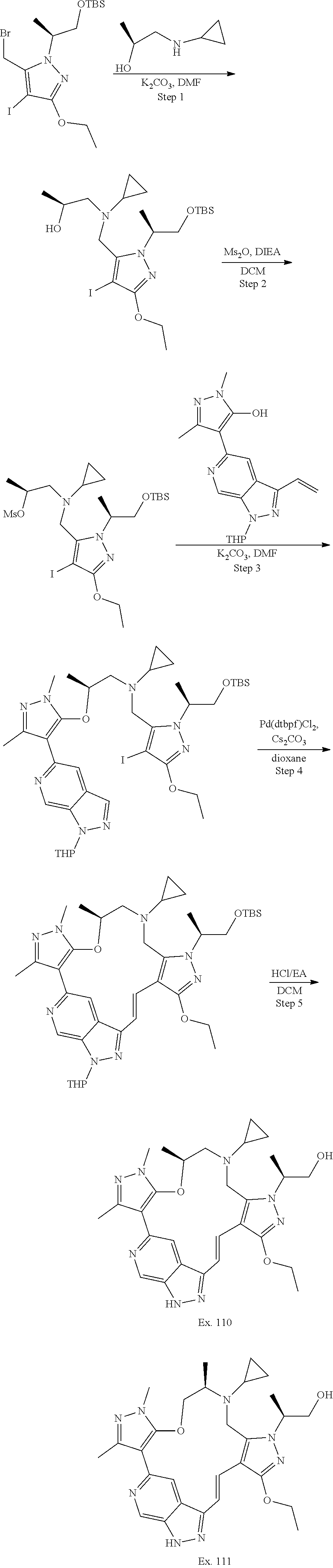

Step 1 through Step 4 were carried out in the same manner as in Ex. 107 using [(2S)-2-[5-(bromomethyl)-3-ethoxy-4-iodo-pyrazol-1-yl]propoxy]-tert-butyl-dimethyl-silane, which was prepared according to the method described in Ex. 19, and commercially available (2S)-1-(cyclopropylamino)propan-2-ol in Step 1, DIEA in step 2, and 2,5-dimethyl-4-(1-tetrahydropyran-2-yl-3-vinyl-pyrazolo[3,4-c]pyridin-5-yl)pyrazol-3-ol, which was prepared according to the method described in Ex. 1, in Step 3. Step 5 was carried out in the same manner as in Ex. 109 to afford Ex. 110 and byproduct Ex. 111. ^{J}H NMR for Ex. 110 and Ex. 111 are in the below NMR table.

### Preparation of 2-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 113) and 2-[(11R,17E)-12-cyclopropyl-16-ethoxy-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 114)

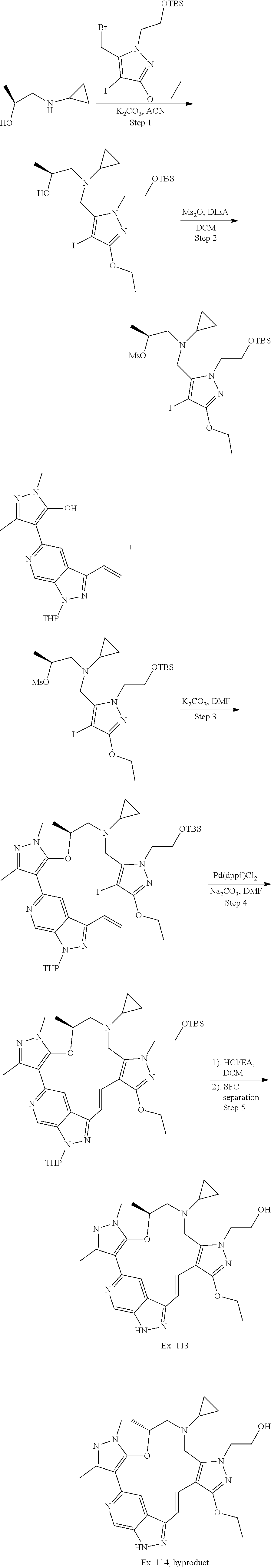

2-[(10S,17E)-12-cyclopropyl-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 113) and 2-[(11R,17E)-12-cyclopropyl-16-ethoxy-6,8,11-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 114) were prepared according to the method described in Ex. 107 substituting Na₂CO₃ and DMF for the base and solvent in step 4 respectively, and using 2-[5-(bromomethyl)-3-ethoxy-4-iodo-pyrazol-1-yl]ethoxy-tert-butyl-dimethyl-silane and 2,5-dimethyl-4-(1-tetrahydropyran-2-yl-3-vinyl-pyrazolo[3,4-c]pyridin-5-yl)pyrazol-3-ol prepared according to the method described in Ex. 1. ¹H NMR for Ex. 113 and Ex. 114 are shown in the below NMR table.

### Preparation of (2S)-2-[(10S,17E)-12-cyclopropyl-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 127) and (2S)-2-[(11R,17E)-12-cyclopropyl-6,8,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 128)

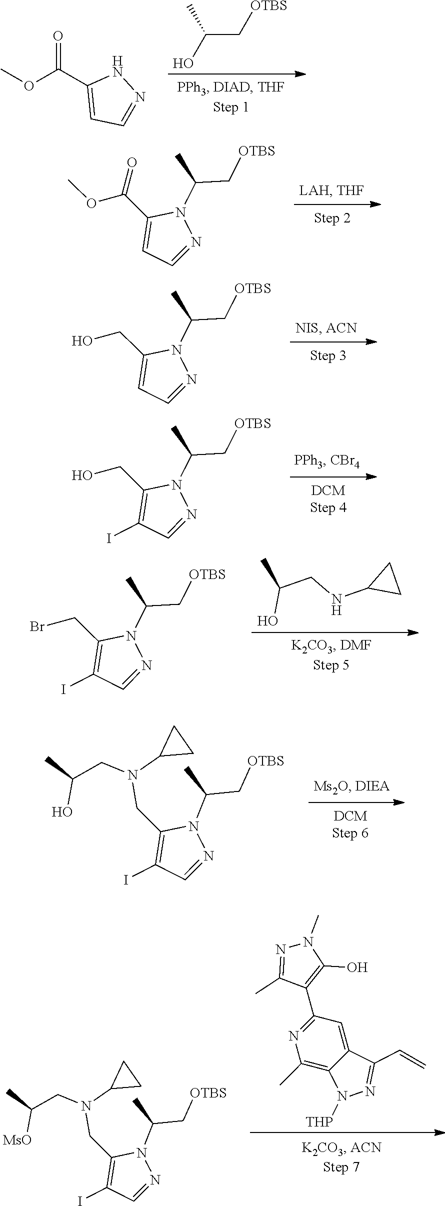

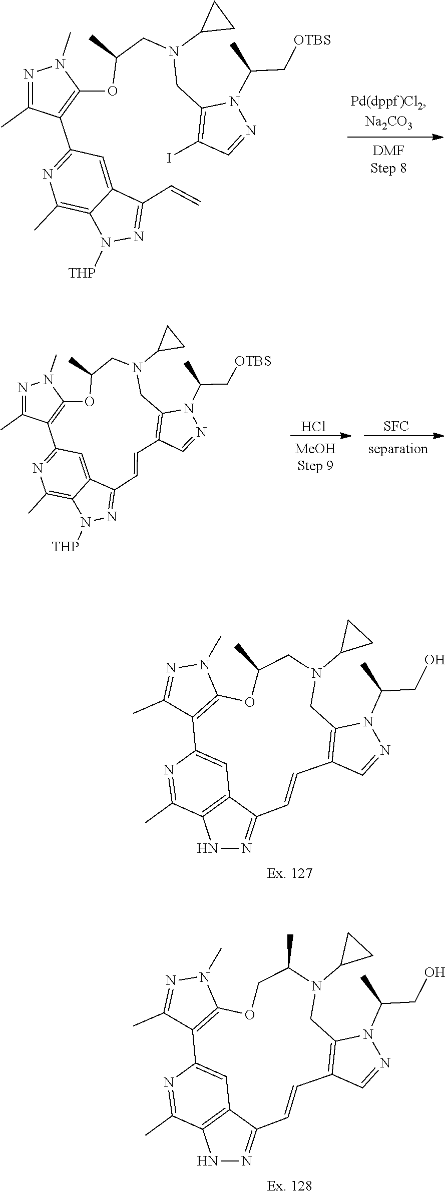

Step 1. To the mixture of commercially available methyl 1H-pyrazole-5-carboxylate (10.0 g, 79.3 mmol, 1 eq), (2R)-1-[tert-butyl(dimethyl)silyl]oxypropan-2-ol, which was prepared according to the method described in Ex. 19, (22.6 g, 119 mmol, 1.5 eq), PPh₃ (41.6 g, 158 mmol, 2 eq) and DIAD (40.1 g, 198 mmol, 2.5 eq) was added THE (300 mL) dropwise at 0° C., and the resulting mixture was stirred for another 2 h at 25° C. under N₂. On completion, the reaction mixture was concentrated in vacuo to give a residue. The residue was purified by column chromatography on silica gel (120 g silica gel, EA in Petroleum ether from 0% to 100%) to give methyl 2-[(1S)-2-[tert-butyl(dimethyl)silyl]oxy-1-methyl-ethyl]pyrazole-3-carboxylate (22.4 g, 92% yield) as yellow oil. ¹H NMR (400 MHz, DMSO-d₆) δ=7.64-7.55 (m, 1H), 6.84-6.81 (m, 1H), 5.45 (qd, J=6.8, 13.2 Hz, 1H), 3.82-3.70 (m, 5H), 1.39 (d, J=6.8 Hz, 3H), 0.72 (s, 9H), −0.07-0.24 (m, 6H). LCMS: (M+1: 299.1).

Step 2. To a solution of methyl 2-[(1S)-2-[tert-butyl(dimethyl)silyl]oxy-1-methyl-ethyl]pyrazole-3-carboxylate (21.0 g, 70.3 mmol, 1 eq) in THE (200 mL) was added LAH (2.5 M, 30.9 mL, 1.1 eq) at 0° C. The mixture was stirred at 25° C. for 1 h. On completion, the mixture was slowly added to water (3 mL) at 0° C., 15% sodium hydroxide solution (3 mL) and water (9 mL) to quench at 0° C. The reaction mixture was filtered and concentrated under reduced pressure to give [2-[(1S)-2-[tert-butyl(dimethyl)silyl]oxy-1-methyl-ethyl]pyrazol-3-yl]methanol (17.0 g, 89% yield) as a colorless oil. LCMS: (M+1: 271.1).

Step 3. To a solution of [2-[(1S)-2-[tert-butyl(dimethyl)silyl]oxy-1-methyl-ethyl]pyrazol-3-yl]methanol (16.0 g, 59.1 mmol, 1 eq) in ACN (160 mL) was added NIS (14.6 g, 65.1 mmol, 1.1 eq) at 0° C. for 0.5 h. The mixture was stirred at 25° C. for 1.5 h. On completion, the reaction mixture was quenched by addition of saturated sodium sulfite solution (100 mL) at 0° C., and then diluted with H₂O (300 mL) and extracted with EA (150 mL×3). The combined organic layers were dried over Na₂SO₄, filtered, and concentrated under reduced pressure to give a residue. The residue was purified by column chromatography on silica gel (220 g silica gel, EA in Petroleum ether from 0% to 100%) to give [2-[(1S)-2-[tert-butyl(dimethyl)silyl]oxy-1-methyl-ethyl]-4-iodo-pyrazol-3-yl]methanol (17.0 g, 72% yield) as a colorless oil. LCMS: (M+1: 397.0).

Step 4. To a solution of [2-[(1S)-2-[tert-butyl(dimethyl)silyl]oxy-1-methyl-ethyl]-4-iodo-pyrazol-3-yl]methanol (17.0 g, 42.9 mmol, 1 eq) in DCM (170 mL) was added CBr₄ (17.1 g, 51.5 mmol, 1.2 eq) at 0° C. under N₂, and then added PPh₃ (13.5 g, 51.5 mmol, 1.2 eq) at 0° C. The mixture was stirred at 25° C. for 1 h. On completion, the reaction mixture was concentrated in vacuo to give a residue. The residue was purified by column chromatography on silica gel (80 g silica gel, EA in Petroleum ether from 0% to 100%) to give [(2S)-2-[5-(bromomethyl)-4-iodo-pyrazol-1-yl]propoxy|-tert-butyl-dimethyl-silane (16.0 g, 81% yield) as a yellow oil. LCMS: (M+1: 460.9).

Step 5. To a solution of [(2S)-2-[5-(bromomethyl)-4-iodo-pyrazol-1-yl]propoxy]-tert-butyl-dimethyl-silane (1.50 g, 3.27 mmol, 1 eq) in DMF (15 mL) was added K₂CO₃ (677 mg, 4.90 mmol, 1.5 eq) and commercially available (2S)-1-(cyclopropylamino)propan-2-ol (432 mg, 3.76 mmol, 1.15 eq). The mixture was stirred at 80° C. for 1 hr. On completion, the mixture was diluted with water (40 mL) and extracted with ethyl acetate (15 mL×3). The combined organic phase was washed with saturated sodium chloride solution (15 mL×2) and dried over Na₂SO₄, filtered and the filtrate was concentrated to give a residue. The residue was purified by column chromatography on silica gel (4 g silica gel, EA in Petroleum ether from 0% to 100%) to give (2S)-1-[[2-[(1S)-2-[tert-butyl(dimethyl)silyl]oxy-1-methyl-ethyl]-4-iodo-pyrazol-3-yl]methyl-cyclopropyl-amino]propan-2-ol (1.58 g, 3.00 mmol, 91% yield, 93% purity) as a yellow oil. LCMS: (M+1: 494.1).

Step 6. To a solution of the product from Step 5 (1.40 g, 2.84 mmol, 1 eq) in DCM (14 mL) was added DIEA (1.10 g, 8.51 mmol, 1.48 mL, 3 eq) and methylsulfonyl methanesulfonate (988 mg, 5.67 mmol, 2 eq) at 0° C. The mixture was stirred at 25° C. for 1 hr. On completion, the mixture was diluted with water (40 mL) and extracted with DCM (15 mL×3). The combined organic phase was washed with saturated sodium chloride solution (10 mL×2) and dried over Na₂SO₄, filtered and the filtrate was concentrated to give [(1S)-2-[[2-[(1S)-2-[tert-butyl(dimethyl)silyl]oxy-1-methyl-ethyl]-4-iodo-pyrazol-3-yl]methyl-cyclopropyl-amino]-1-methyl-ethyl]methanesulfonate (1.76 g, crude) as a colorless oil. LCMS: (M+1: 572.1).

Step 7. A mixture of the mesylate form Step 6 (1.50 g, 2.62 mmol, 1 eq), 2,5-dimethyl-4-(7-methyl-1-tetrahydropyran-2-yl-3-vinyl-pyrazolo[3,4-c]pyridin-5-yl)pyrazol-3-ol (881 mg, 2.49 mmol, 0.95 eq), which was prepared according to the method described in Ex. 47, and K₂CO₃ (1.09 g, 7.87 mmol, 3 eq) in ACN (15 mL) was stirred at 60° C. for 2 h. On completion, the reaction mixture was concentrated in vacuo to give a residue. The residue was purified by column chromatography on silica gel (12 g silica gel, EA in Petroleum ether from 0% to 100%) to give N-[[2-[(1S)-2-[tert-butyl(dimethyl)silyl]oxy-1-methyl-ethyl]-4-iodo-pyrazol-3-yl]methyl]-N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]cyclopropanamine (1.00 g, 45% yield) as a green solid. LCMS: (M+1: 829.4).

Step 8. To a solution of the product from Step 7 (220 mg, 0.265 mmol, 1 eq) in DMF (20 mL) was added Na₂CO₃ (84.4 mg, 0.796 mmol, 3 eq) and cyclopentyl(diphenyl)phosphane; dichloropalladium; iron (Pd(dppf)Cl₂) (19.4 mg, 0.0265 mmol, 0.1 eq). The mixture was stirred at 80° C. for 16 h under nitrogen atmosphere. On completion, the mixture was poured into H₂O (80 mL) and extracted with EA (100 mL×3). The combined organic phase was washed with H₂O (100 mL×3) and dried over anhydrous sodium sulfate, filtered and concentrated to give a residue. The residue was purified by column chromatography (SiO₂, PE/THF=1:0 to 0:1) to give (10S,17E)-14-[(2S)-1-{[tert-butyl(dimethyl)silyl]oxy}propan-2-yl]-12-cyclopropyl-6,8,10,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (80.0 mg) as a green solid. LCMS: (M+1: 701.4).

Step 9. To a solution of the product from Step 8 (70.0 mg, 99.8 mol, 1 eq) in MeOH (1.5 mL) was added HCl (12 M, 0.5 mL, 60.08 eq). The mixture was stirred at 25° C. for 1 h. On completion, the reaction mixture was concentrated in vacuo to give a residue. The mixture was purified by Prep-HPLC (column: Phenomenex luna C18 150*25 mm*10 um; mobile phase: [water(FA)-ACN]; gradient:15%-45% B over 8 min), followed by further SFC separation (column: REGIS (R,R)WHELK-01 (250 mm*25 mm, 10 um); mobile phase: [CO2—ACN/MeOH(0.1% NH₃H₂O)]; B %:45%, isocratic elution mode) to give Ex. 127 (7.70 mg) as brown solid and a byproduct Ex. 128 (5.02 mg) as brown solid. ¹H NMR for Ex. 127 and Ex. 128 can be found in the below NMR table.

### Preparation of (2S)-2-[(10S,17E)-12-cyclopropyl-6-(methoxymethyl)-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 139) and (2S)-2-[(11R,17E)-12-cyclopropyl-6-(methoxymethyl)-8,11,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 140)

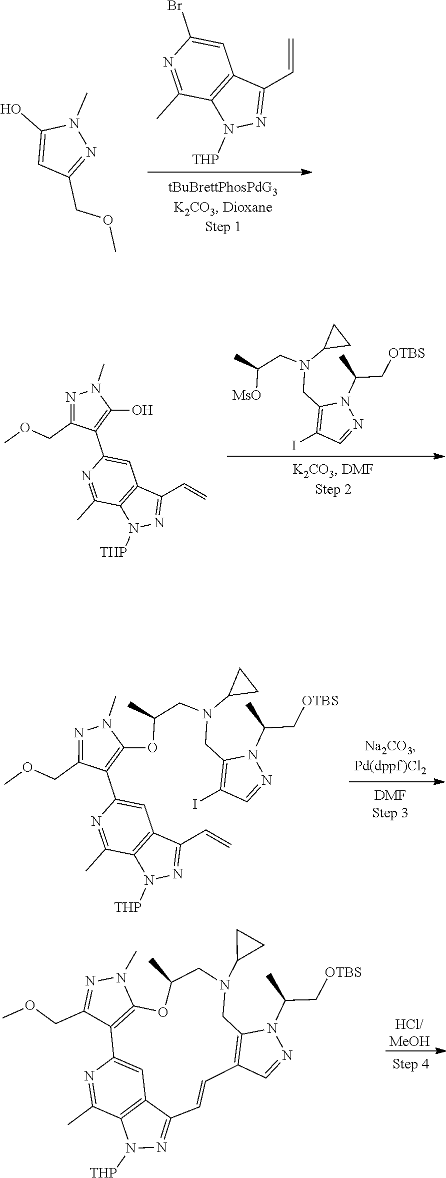

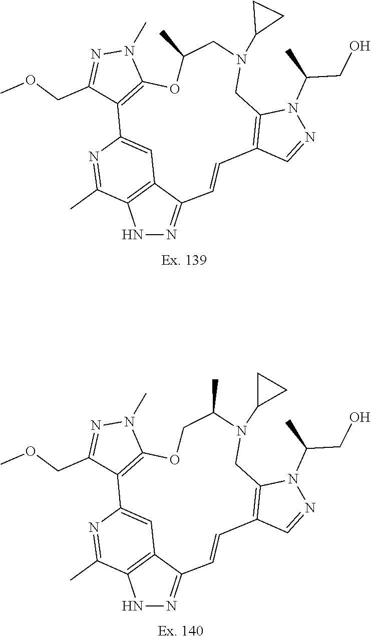

Step 1. To a solution of 5-bromo-7-methyl-1-(tetrahydro-2H-pyran-2-yl)-3-vinyl-1H-pyrazolo[3,4-c]pyridine, which was prepared according to the method described in Ex. 28 (5.40 g, 16.7 mmol, 1 eq), in dioxane (43 mL) was added 3-(methoxymethyl)-1-methyl-1H-pyrazol-5-ol, which was prepared according to the method described in Ex. 130 (2.86 g, 20.1 mmol, 1.2 eq), K₂CO₃ (6.95 g, 50.3 mmol, 3 eq) and tBuBrettphos Pd G3 (1.43 g, 1.68 mmol, 0.1 eq) under N₂ atmosphere. The mixture was stirred at 130° C. for 1 hour under N₂ atmosphere. On completion, the mixture was filtered and concentrated to give a residue. The residue was purified by column chromatography (SiO₂, Dichloromethane:Methanol=1:0 to 10:1) to give 3-(methoxymethyl)-1-methyl-4-(7-methyl-1-(tetrahydro-2H-pyran-2-yl)-3-vinyl-1H-pyrazolo[3,4-c]pyridin-5-yl)-1H-pyrazol-5-ol (5.20 g, 13.5 mmol, 81% yield) as red oil. LCMS: (M+1: 384.2).

Steps 2 through 4 were performed in a similar manner to steps 7 through 9 in the synthesis of Ex. 127/128, with DMF used as the solvent in step 7 rather than ACN. 1H NMR for Ex. 139 and Ex. 140 can be found in the below NMR table.

### Preparation of 2-[(8aR,9S,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol (Ex. 177) and 2-[(8aR,9R,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][[1,4]oxazacyclopentadecin-3(4H)-yl]ethan-1-ol (Ex. 178)

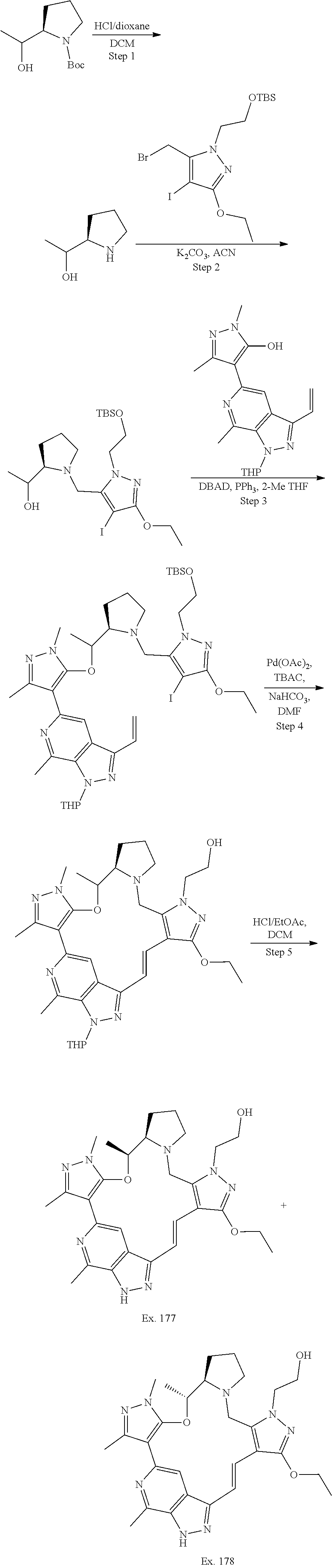

Step 1. To a solution of tert-butyl (2R)-2-(1-hydroxyethyl)pyrrolidine-1-carboxylate, which was prepared according to the method described in Ex. 32/Ex. 33 (5.00 g, 23.2 mmol, 1 eq,) in DCM (50 mL) was added HCl/dioxane (4.00 M, 50 mL, 8.61 eq). The mixture was stirred at 25° C. for 1 hour. On completion, the reaction was concentrated in vacuum to give 1-((R)-pyrrolidin-2-yl)ethan-1-ol (3.50 g, crude) as a yellow solid. ¹H NMR (400 MHz, DMSO-d₅) δ=9.96-9.37 (m, 1H), 8.70 (s, 1H), 3.96 (d, J=4.4, 6.4 Hz, 1H), 3.81-3.69 (m, 1H), 3.13-3.04 (m, 2H), 1.98-1.85 (m, 2H), 1.84-1.74 (m, 1H), 1.64-1.51 (m, 1H), 1.17-1.05 (m, 3H).

Step 2. To a solution of 1-[(2R)-pyrrolidin-2-yl]ethanol (1.00 g, 6.59 mmol, 1 eq, HCl) in ACN (20 mL) was added K₂CO₃ (2.73 g, 19.8 mmol, 3 eq) and (S)-5-(bromomethyl)-1-(1-((tert-butyldimethylsilyl)oxy)propan-2-yl)-3-ethoxy-4-iodo-1H-pyrazole, prepared according to the method described in Ex. 1 (3.23 g, 6.59 mmol, 1 eq). The mixture was stirred at 80° C. for 2 hours. On completion, the reaction solution was filtered to remove the K₂CO₃ and then concentrated in vacuum to give a residue. The residue was purified by column chromatography (SiO₂, Petroleum ether/THF=1:0 to 5:1) to give 1-((R)-1-((1-(2-((tert-butyldimethylsilyl)oxy)ethyl)-3-ethoxy-4-iodo-1H-pyrazol-5-yl)methyl)pyrrolidin-2-yl)ethan-1-ol (2.70 g, 4.74 mmol, 72% yield, 92% purity) as a colorless liquid. ¹H NMR (400 MHz, DMSO-d₆) δ=4.45 (d, J=4.4 Hz, 1H), 4.36-4.21 (m, 1H), 4.18-4.08 (m, 3H), 4.06-3.96 (m, 1H), 3.81 (t, J=4.8 Hz, 2H), 3.72-3.55 (m, 1H), 2.74-2.62 (m, 1H), 2.46-2.39 (m, 1H), 2.25 (d, J=6.4, 9.2 Hz, 1H), 1.75-1.63 (m, 2H), 1.62-1.43 (m, 2H), 1.28 (t, J=7.2 Hz, 3H), 1.19-1.05 (m, 1H), 1.01 (d, J=1.2, 6.0 Hz, 3H), 0.77 (d, J=2.4 Hz, 9H), −0.09 (s, 3H), −0.10-0.13 (m, 3H); LCMS: (M+1: 524.4).

Step 3. A solution of 1-((R)-1-((1-(2-((tert-butyldimethylsilyl)oxy)ethyl)-3-ethoxy-4-iodo-1H-pyrazol-5-yl)methyl)pyrrolidin-2-yl)ethan-1-ol (926 mg, 1.77 mmol, 1 eq), 1,3-dimethyl-4-(7-methyl-1-(tetrahydro-2H-pyran-2-yl)-3-vinyl-1H-pyrazolo[3,4-c]pyridin-5-yl)-1H-pyrazol-5-ol, which was prepared according to the method described in Ex. 47 (500 mg, 1.41 mmol, 0.8 eq), and PPh₃ (927 mg, 3.54 mmol, 2 eq) in 2-methyltetrahydrofuran (10 mL) was degassed and purged with N₂ atmosphere for 3 times. Then DBAD (896 mg, 3.89 mmol, 2.2 eq) was added, and the mixture was stirred at 25° C. for 2 hours under N₂ atmosphere. On completion, the reaction was concentrated in vacuum to give a residue. The residue was purified by column chromatography (SiO₂, Petroleum ether/THF=1:0 to 3:1) to give 5-(5-(1-((R)-1-((1-(2-((tert-butyldimethylsilyl)oxy)ethyl)-3-ethoxy-4-iodo-1H-pyrazol-5-yl)methyl)pyrrolidin-2-yl)ethoxy)-1,3-dimethyl-1H-pyrazol-4-yl)-7-methyl-1-(tetrahydro-2H-pyran-2-yl)-3-vinyl-1H-pyrazolo[3,4-c]pyridine (750 mg, 0.655 mmol, 37% yield, 75% purity) as an orange oil. LCMS: (M+1: 859.3).

Step 4. To a solution of 5-(5-(1-((R)-1-((1-(2-((tert-butyldimethylsilyl)oxy)ethyl)-3-ethoxy-4-iodo-1H-pyrazol-5-yl)methyl)pyrrolidin-2-yl)ethoxy)-1,3-dimethyl-1H-pyrazol-4-yl)-7-methyl-1-(tetrahydro-2H-pyran-2-yl)-3-vinyl-1H-pyrazolo[3,4-c]pyridine (700 mg, 0.815 mmol, 1 eq) in DMF (7 mL) was added NaHCO₃ (342 mg, 4.07 mmol, 5 eq), TBAC (453 mg, 1.63 mmol, 2 eq) and Pd(OAc)₂ (27.5 mg, 0.122 mmol, 0.15 eq). The mixture was stirred at 130° C. for 1 hour under N₂ atmosphere. On completion, the reaction mixture diluted with H₂O (100 mL) and extracted with EA (50 mL×3). The combined organic phase was dried over anhydrous sodium sulfate, filtered, and concentrated under reduced pressure to give a residue. The residue was purified by column chromatography (SiO₂, DCM/MeOH=1:0 to 10:1) to give 2-[(8aR,19E)-1-ethoxy-9,11,13,21-tetramethyl-17-(oxan-2-yl)-7,8,8a,9,11,17-hexahydro-6H-14,16-(azenometheno)tripyrazolo[3,4-j3′,4′-j:4″,3″-n]pyrrolo[2,1-c][1,4]oxazacyclopentadecin-3(4H)-yl]ethan-1-ol (380 mg, 0.616 mmol, 53% yield, 70% purity) as a black brown oil. LCMS: (M+1: 617.3).

Step 5. To a solution of 2-[(8aR,19E)-1-ethoxy-9,11,13,21-tetramethyl-17-(oxan-2-yl)-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol (340 mg, 0.551 mmol, 1 eq) in DCM (4 mL) was added HCl/EtOAc (4.00 M, 0.138 mL, 1 eq). The mixture was stirred at 25° C. for 1 hour. On completion, the reaction was concentrated in vacuum to give a residue. The residue was purified by prep-HPLC to give 2-[(8aR,9S,19E)-1-ethoxy-9,11,13,21-tetramethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]ethan-1-ol (15.35 mg, 0.029 mmol, 5.21% yield, 99.69% purity) as a white solid (Ex. 177) and 2-[(8aR,9R,19E)-1-ethoxy-9,11,13,21-tetramethyl-7, 8,8a,9,11,17-hexahydro-6H-14,16-(azenometheno)tripyrazolo[3,4-f:3′,4′-j:4″,3″-n|pyrrolo[2,1-c∥1,4]oxazacyclopentadecin-3(4H)-yl]ethan-1-ol (12.24 mg, 0.021 mmol, 3.74% yield, 89.61% purity) as a white solid (Ex. 178). ¹H NMR for Ex. 177 and Ex. 178 can be found in the below NMR table.

Ex. 179 and Ex. 180 were synthesized following the procedure described for the synthesis of Ex. 177/Ex. 178, using (S)-5-(bromomethyl)-1-(1-((tert-butyldimethylsilyl)oxy)propan-2-yl)-3-ethoxy-4-iodo-1H-pyrazole (which was prepared following the method described in Ex. 19) in Step 2. ¹H NMR for Ex. 179 and Ex. 180 can be found in the below NMR table.

General Method B: using Ex. 22 as example.

### Preparation of {[(10S,17E)-14-(2-hydroxyethyl)-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 22)

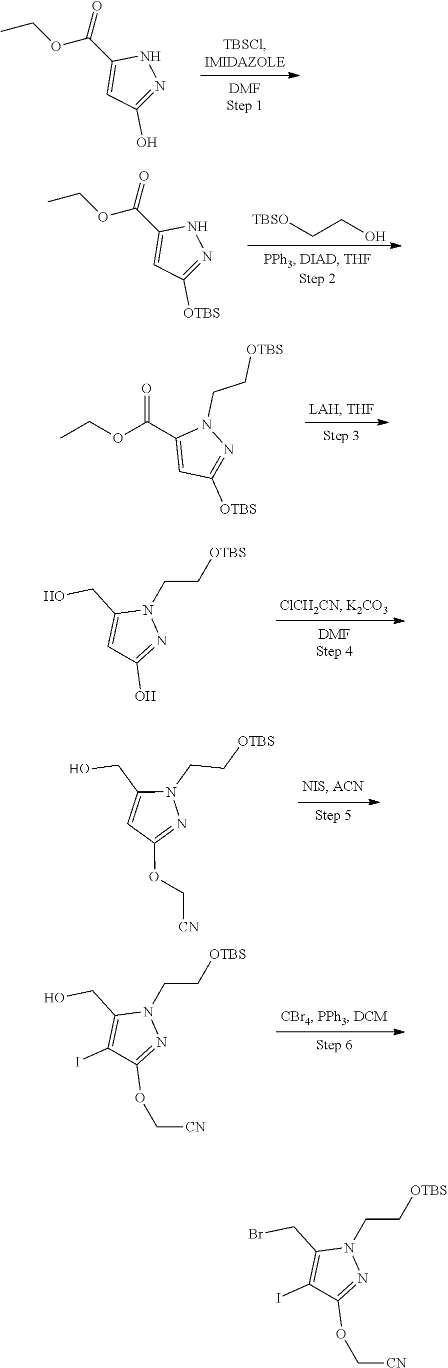

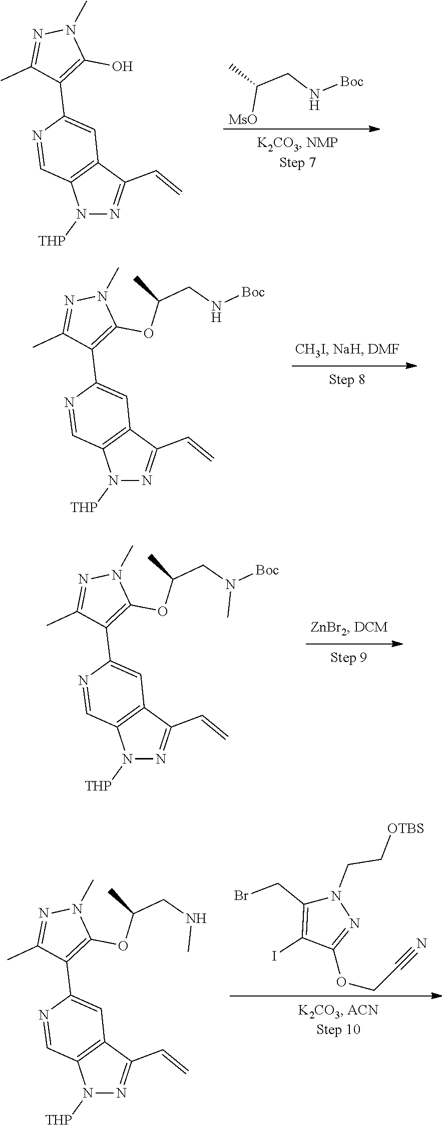

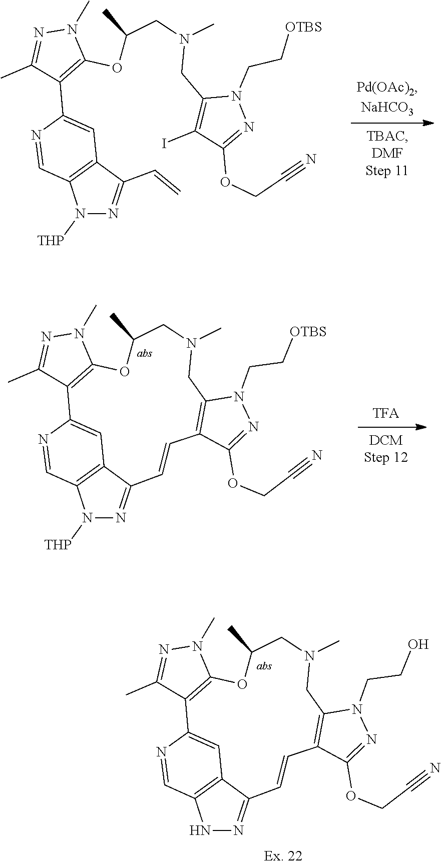

Step 1. To a solution of commercially available ethyl 3-hydroxy-1H-pyrazole-5-carboxylate (1.00 g, 6.40 mmol, 1 eq) in DMF (10 mL) was added imidazole (1.31 g, 19.2 mmol, 3 eq), and then TBSCl (1.45 g, 9.61 mmol, 1.5 eq) was added at 0° C. under N₂. The mixture was stirred at 25° C. for 12 h. On completion, the reaction mixture was partitioned between EA (30 mL×3) and water (30 mL). The combined organic phase was dried over anhydrous sodium sulfate, filtered, and concentrated to give a residue. The residue was purified by column chromatography (SiO₂, PE:THF=1:1 to 1:1) to give ethyl 3-{[tert-butyl(dimethyl)silyl]oxy}-1H-pyrazole-5-carboxylate (1.70 g, 6.29 mmol, 98% yield) as a white solid. LCMS: (M+1: 271.1).

Step 2. To a solution of ethyl 3-{[tert-butyl(dimethyl)silyl]oxy}-1H-pyrazole-5-carboxylate (1.70 g, 6.29 mmol, 1 eq) in THE (17 mL) was added commercially available 2-[tert-butyl(dimethyl)silyl]oxyethanol (1.11 g, 6.29 mmol, 1 eq) and PPh₃ (3.63 g, 13.8 mmol, 2.2 eq), then DIAD (3.18 g, 15.7 mmol, 2.5 eq) was added at 0° C. under N₂. The mixture was stirred at 25° C. for 2 h. On completion, the mixture was filtered and concentrated to give a residue. The residue was purified by column chromatography (SiO₂, PE/EA=1:1 to 1:1) to give ethyl 5-[tert-butyl(dimethyl)silyl]oxy-2-[2-[tert-butyl(dimethyl)silyl]oxyethyl]pyrazole-3-carboxy late(2.57 g, 5.99 mmol, 95% yield) as a brown solid. LCMS: (M+1: 429.6).

Step 3. To a solution of ethyl 5-[tert-butyl(dimethyl)silyl]oxy-2-[2-[tert-butyl(dimethyl) silyl]oxyethyl]pyrazole-3-carboxylate (2.27 g, 5.29 mmol, 1 eq) in THE (22 mL) was added LiAlH₄ (2.5 M, 4.24 mL, 2 eq) at 0° C. The mixture was stirred at 25° C. for 2 h. On completion, water (0.4 mL) was added to the reaction solution, then 15% NaOH (0.4 mL) was added to the solution, then water (1.2 mL) was added, the mixture was filtered and concentrated to give a residue. The residue was purified by column chromatography (SiO₂, Petroleum ether/Ethyl acetate=1:1 to 1:1) to give 1-[2-[tert-butyl(dimethyl)silyl]oxyethyl]-5-(hydroxymethyl)pyrazol-3-ol (500 mg, 1.84 mmol, 35% yield) as a white solid. LCMS: (M+1: 273.1).

Step 4. To a solution of 1-[2-[tert-butyl(dimethyl)silyl]oxyethyl]-5-(hydroxymethyl)pyrazol-3-ol (450 mg, 1.65 mmol, 1 eq) in DMF (5 mL) was added 2-chloroacetonitrile (187 mg, 2.48 mmol, 1.5 eq) and K₂CO₃ (457 mg, 3.30 mmol, 2 eq). The mixture was stirred at 80° C. for 2 h. On completion, the reaction mixture was partitioned between EA (15 mL×3) and water (15 mL). The combined organic phase was dried over anhydrous sodium sulfate, filtered, and concentrated to give a residue. The residue was purified by column chromatography (SiO₂, PE:THF=2:1 to 2:1) to give 2-[1-[2-[tert-butyl(dimethyl)silyl]oxyethyl]-5-(hydroxymethyl)pyrazol-3-yl]oxyacetonitrile (380 mg, 1.22 mmol, 74% yield) as a white solid. LCMS: (M+1: 312.1).

Step 5. To a solution of 2-[1-[2-[tert-butyl(dimethyl)silyl]oxyethyl]-5-(hydroxymethyl)pyrazol-3-yl]oxyacetonitrile (350 mg, 1.12 mmol, 1 eq) in ACN (4 mL) was added NIS (379 mg, 1.69 mmol, 1.5 eq) 0° C. The mixture was stirred at 25° C. for 2 h. On completion, the reaction mixture was partitioned between EA (5 mL×3) and water (5 mL). The combined organic phase was dried over anhydrous sodium sulfate, filtered and concentrated to give a residue. The residue was purified by column chromatography (SiO₂, PE:THF=2:1 to 2:1) to give 2-[1-[2-[tert-butyl(dimethyl)silyl]oxyethyl]-5-(hydroxylmethyl)-4-iodo-pyrazol-3-yl]oxyacetonitrile (429 mg, 981 mol, 87% yield) as a white solid. LCMS: (M+1: 438.0).

Step 6. To a solution of 2-[1-[2-[tert-butyl(dimethyl)silyl]oxyethyl]-5-(hydroxymethyl)-4-iodo-pyrazol-3-yl]oxyacetonitrile (389 mg, 889 μmol, 1 eq) in DCM (4 mL) was added PPh₃ (280 mg, 1.07 mmol, 1.2 eq), then CBr₄ (354 mg, 1.07 mmol, 1.2 eq) was added at 0° C. under N₂. The mixture was stirred at 25° C. for 2 h. On completion, the mixture was filtered and concentrated to give a residue. The residue was purified by column chromatography (SiO₂, PE:THF=3:1 to 3:1) to give 2-[5-(bromomethyl)-1-[2-[tert-butyl(dimethyl)silyl]oxyethyl]-4-iodo-pyrazol-3-yl]oxyacetonitrile (350 mg, 699 mol, 78% yield) as a yellow oil. LCMS: (M+1: 501.9).

Step 7. To a solution of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (550 mg, 1.62 mmol, 1 eq) in NMP (5 mL) was added commercially available [(1R)-2-(tert-butoxycarbonylamino)-1-methyl-ethyl]methanesulfonate (1.03 g, 4.05 mmol, 2.5 eq) and K₂CO₃ (224 mg, 1.62 mmol, 1 eq). The mixture was stirred at 60° C. for 2 h. On completion, the reaction mixture was partitioned between 2-Me-THF (20 mL×3) and water (20 mL). The combined organic phase was dried over anhydrous sodium sulfate, filtered and concentrated to give a residue. The residue was purified by column chromatography (SiO₂, PE:THF=1:1 to 1:1) to give tert-butyl N-[(2S)-2-[2,5-dimethyl-4-(1-tetrahydropyran-2-yl-3-vinyl-pyrazolo[3,4-c]pyridin-5-yl)pyrazol-3-yl]oxypropyl]carbamate (252 mg, 507 mol, 31% yield) as a white solid. LCMS: (M+1: 497.2).

Step 8. To a solution of tert-butyl N-[(2S)-2-[2,5-dimethyl-4-(1-tetrahydropyran-2-yl-3-vinyl-pyrazolo[3,4-c]pyridin-5-yl)pyrazol-3-yl]oxypropyl]carbamate (232 mg, 467 mol, 1 eq) in DMF (5 mL) was added NaH (37.4 mg, 934 μmol, 60% purity, 2 eq) at 0° C., the mixture was stirred at 0° C. for 0.5 h, then CH₃I (99.5 mg, 701 mol, 1.5 eq) was added. The mixture was stirred at 25° C. for 2 h. On completion, saturated ammonium chloride (1 mL) was added to quench the reaction mixture, the mixture was partitioned between EA (15 mL×3) and water (15 mL), and the combined organic phase was dried over anhydrous sodium sulfate, filtered and concentrated to give a residue. The residue was purified by column chromatography (SiO₂, PE:THF=1:1 to 1:1) to give tert-butyl N-[(2S)-2-[2,5-dimethyl-4-(1-tetrahydropyran-2-yl-3-vinyl-pyrazolo[3,4-c]pyridin-5-yl)pyrazol-3-yl]oxypropyl]-N-methyl-carbamate (183 mg, 358 mol, 77% yield) as a white solid. LCMS: (M+1: 511.3).

Step 9. To a solution of tert-butyl N-[(2S)-2-[2,5-dimethyl-4-(1-tetrahydropyran-2-yl-3-vinyl-pyrazolo[3,4-c]pyridin-5-yl)pyrazol-3-yl]oxypropyl]-N-methyl-carbamate (183 mg, 358 mol, 1 eq) in DCM (2 mL) was added ZnBr₂ (403 mg, 1.79 mmol, 5 eq). The mixture was stirred at 25° C. for 12 h. On completion, the reaction mixture was partitioned between DCM (2 mL×3) and water (2 mL). The combined organic phase was dried over anhydrous sodium sulfate, filtered and concentrated to give a residue. The residue was purified by column chromatography (SiO₂, DCM:MeOH=5:1 to 5:1) to give (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (77.0 mg, 187 mol, 52% yield) as a white solid. LCMS: (M+1: 411.1).

Step 10. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (190 mg, 463 mol, 1 eq) in ACN (2 mL) was added 2-[5-(bromomethyl)-1-[2-[tert-butyl(dimethyl)silyl]oxyethyl]-4-iodo-pyrazol-3-yl]oxyacetonitrile (254 mg, 509 μmol, 1.1 eq) and K₂CO₃ (192 mg, 1.39 mmol, 3 eq). The mixture was stirred at 60° C. for 1 h. On completion, the mixture was filtered and concentrated to give a residue. The residue was purified by column chromatography (SiO₂, PE:THF=1:1 to 1:1) to give 2-[1-[2-[tert-butyl(dimethyl)silyl]oxyethyl]-5-[[[(2S)-2-[2,5-dimethyl-4-(1-tetrahydropyran-2-yl-3-vinyl-pyrazolo[3,4-c]pyridin-5-yl)pyrazol-3-yl]oxypropyl]-methyl-amino]methyl]-4-iodo-pyrazol-3-yl]oxyacetonitrile (329 mg, 396 mol, 86% yield) as a white solid. LCMS: (M+1: 830.1).

Step 11. To a mixture of 2-11-12-|tert-butyl(dimethyl)silyl]oxyethyl]-5-∥[(2S)-2-12,5-dimethyl-4-(1-tetrahydropyran-2-yl-3-vinyl-pyrazolo[3,4-c]pyridin-5-yl)pyrazol-3-yl]oxypropyl]-methyl-amino]methyl]-4-iodo-pyrazol-3-yl]oxyacetonitrile (309 mg, 372 μmol, 1 eq) in DMF (15 mL) was added NaHCO₃ (78.2 mg, 931 μmol, 2.5 eq), Pd(OAc)₂ (16.7 mg, 74.5 μmol, 0.2 eq) and TBAC (103 mg, 372 mol, 1 eq) degassed and purged with N₂ for 3 times, then the mixture was stirred at 130° C. for 0.5 h under N₂ atmosphere. On completion, the reaction mixture was partitioned between EA (45 mL×3) and water (45 mL). The combined organic phase was dried over anhydrous sodium sulfate, filtered, and concentrated to give a residue. The residue was purified by column chromatography (SiO₂, PE:THF=1:1 to 1:1) to give {[(10S,17E)-14-(2-{[tert-butyl(dimethyl)silyl]oxy}ethyl)-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (223 mg, 317 μmol, 85% yield) as a white solid. LCMS: (M+1: 702.3).

Step 12. To a solution of {[(10S,17E)-14-(2-{[tert-butyl(dimethyl)silyl]oxy}ethyl)-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (203 mg, 289 μmol, 1 eq) in DCM (2 mL) was added TFA (3.07 g, 26.9 mmol, 93 eq). The mixture was stirred at 25° C. for 1 h. On completion, the mixture was concentrated to give a residue. The residue was purified by column chromatography (SiO₂, DCM:MeOH=10:1 to 10:1) to give Ex. 22 (52.78 mg, 105 μmol, 36.24% yield) as yellow solid. LCMS: (M+1: 504.1). ¹H NMR for Ex. 22 is shown in the below NMR table.

### Preparation of (2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 27)

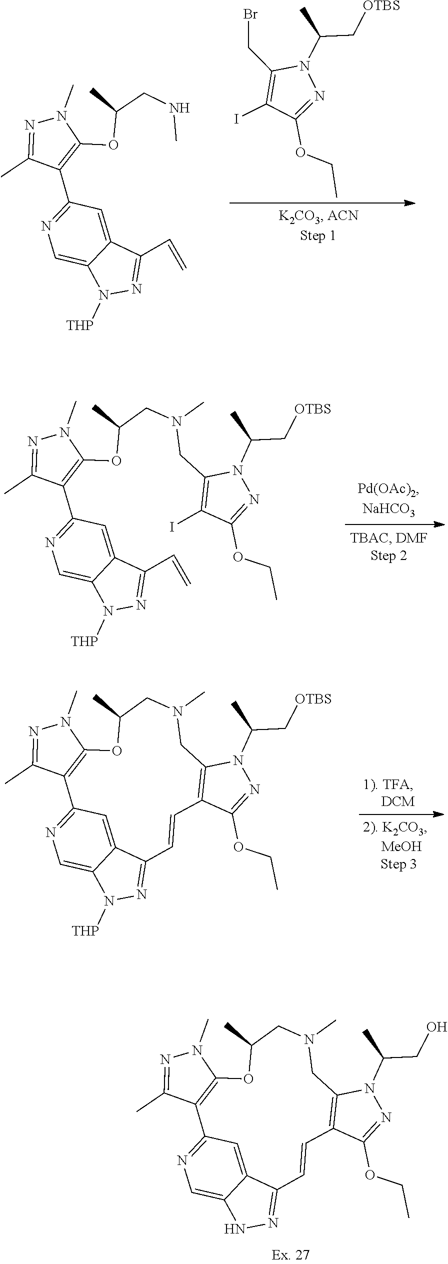

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (150 mg, 365 mol, 1 eq) and [(2S)-2-[5-(bromomethyl)-3-ethoxy-4-iodo-pyrazol-1-yl]propoxy]-tert-butyl-dimethyl-silane, which was prepared according to the method described in Ex. 19, (221 mg, 438 μmol, 1.2 eq) in ACN (2 mL) was added K₂CO₃ (152 mg, 1.10 mmol, 3 eq). The mixture was stirred at 60° C. for 2 h. On completion, the mixture was filtered and concentrated to give a residue. The residue was purified by column chromatography (SiO₂, PE/THF=1:0 to 2:1) to give (2S)-N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (226 mg, 271 mol, 74% yield) as a colourless oil. LCMS: (M+1:833.2).

Step 2. A mixture of (2S)-N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (206 mg, 247.3 mol, 1 eq) in DMF (11 mL) was added TBAC (68.7 mg, 247 μmol, 1 eq), NaHCO₃ (51.9 mg, 618 μmol, 2.5 eq) and Pd(OAc)₂ (11.1 mg, 49.5 μmol, 0.2 eq), the mixture was degassed and purged with N₂ for 3 times, and then the mixture was stirred at 140° C. for 1 h under N₂ atmosphere. On completion, the mixture was quenched with water (22 mL) and extracted with ethyl acetate (15 mL×3). The combined organic phase was dried over anhydrous sodium sulfate, filtered and concentrated to give a residue. The residue was purified by column chromatography (SiO₂, PE/EA=1:0 to 1:1) to give (10S,17E)-14-[(2S)-1-{[tert-butyl(dimethyl)silyl]oxy}propan-2-yl]-16-ethoxy-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (118 mg, 167 μmol, 68% yield) as a yellow oil. LCMS: (M+1:705.3).

Step 3. To a solution of (10S,17E)-14-[(2S)-1-{[tert-butyl(dimethyl)silyl]oxy}propan-2-yl]-16-ethoxy-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (108 mg, 153 μmol, 1 eq) in DCM (1 mL) was added TFA (1.54 g, 13.5 mmol, 87.9 eq). The mixture was stirred at 25° C. for 1 h. On completion, the mixture was concentrated to give 90 mg of crude yellow solid. LCMS: (M+1: 603.3). This 90 mg of yellow solid obtained was then dissolved in MeOH (2 mL) and K₂CO₃ (206 mg, 1.49 mmol, 10 eq) was added. The mixture was stirred at 25° C. for 0.5 h. On completion, the mixture was filtered and concentrated to give a residue. The crude product was purified by reverse-phase HPLC(C18, ACN/0.1% FA=1:0 to 1:1) to give Ex. 27 (37.67 mg, 74.36 mol, 49.79% yield) as a white solid. LCMS: (M+1: 507.2). ¹H NMR for Ex. 27 is shown in the below NMR table.

### Preparation of 2-[(10S,17E)-16-ethoxy-8,10,12,20-tetramethyl-2,8,10,11,12,13-hexahydro-14H-5,3-(azenometheno)tripyrazolo[3,4-f:3′,4′-j:4″,3″-30n][1,4]oxazacyclopentadecin-14-yl]ethan-1-ol (Ex. 29)

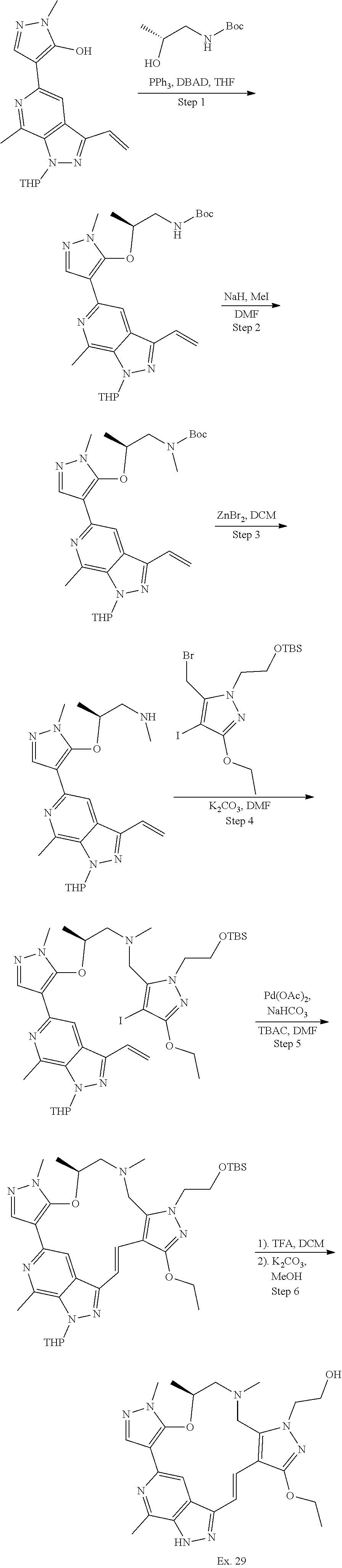

Step 1. A mixture of 2-methyl-4-(7-methyl-1-tetrahydropyran-2-yl-3-vinyl-pyrazolo[3,4-c]pyridin-5-yl)pyrazol-3-ol, which was prepared according to the method described in Ex. 28, (1.85 g, 5.45 mmol, 1 eq), commercially available tert-butyl N-[(2R)-2-hydroxypropyl]carbamate (1.05 g, 6.00 mmol, 1.1 eq), PPh₃ (3.15 g, 11.9 mmol, 2.2 eq), DBAD (2.76 g, 11.99 mmol, 2.2 eq) in THE (2 mL) was stirred at 25° C. for 1 h. On completion, the mixture was concentrated in vacuum. The residue was purified by column chromatography (SiO₂, Petroleum ether/THF=1/0 to 5/1) to give tert-butyl N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (3.20 g, 2.26 mmol, 41% yield, 92% purity) as a yellow solid. LCMS: (M+1: 497.5).

Step 2. To a solution of tert-butyl N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (3.00 g, 6.04 mmol, 1 eq) in DMF (40 mL) was added NaH (483 mg, 12.1 mmol, 60% purity, 2 eq) at 0° C. for 0.5 h, then MeI (1.71 g, 12.1 mmol, 752 μL, 2 eq) was added at 0° C. The mixture was stirred at 25° C. for 1 h. On completion, to the mixture was slowly added water (50 mL), and extracted with EA (3×20 mL). The combined organic layers were washed with brine (2×100 mL), dried over Na₂SO₄, filtered and concentrated to give tert-butyl N-methyl-N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (3.20 g, 2.19 mmol, 36% yield) as a yellow solid. LCMS: (M+1: 511.5).

Step 3. To a solution of tert-butyl N-methyl-N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (3.20 g, 3.13 mmol, 1 eq), ZnBr₂ (2.82 g, 12.5 mmol, 4 eq) in DCM (30 mL). The mixture was stirred at 25° C. for 1 h. On completion, the mixture was concentrated in vacuum. The residue was purified by column chromatography (SiO₂, Petroleum ether/THF=1/0 to 5/1) to give (2S)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (2.57 g, 2.19 mmol, 69% yield, 58% purity) as a yellow solid. LCMS: (M+1: 411.2).

Step 4. A mixture of (2S)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (2.50 g, 1.89 mmol, 1 eq), K₂CO₃ (782 mg, 5.66 mmol, 3 eq), 2-[5-(bromomethyl)-3-ethoxy-4-iodo-pyrazol-1-yl]ethoxy-tert-butyl-dimethyl-silane, prepared according to the method described in Ex. 1, (1.02 g, 2.08 mmol, 1.1 eq) in DMF (30 mL) was stirred at 80° C. for 1 h. On completion, the mixture was quenched with water (30 mL) and extracted with ethyl acetate (15 mL×3). The combined organic phase was dried over anhydrous sodium sulfate, filtered and concentrated to give a residue. The residue was purified by column chromatography (SiO₂, Petroleum ether/THF=1/0 to 5/1) to give (2S)-N-[[2-[2-[tert-butyl(dimethyl)silyl]oxyethyl]-5-ethoxy-4-iodo-pyrazol-3-yl]methyl]-N-methyl-2-|2-methyl-4-(7-methyl-1-tetrahydropyran-2-yl-3-vinyl-pyrazolo[3,4-c]pyridin-5-yl)pyrazol-3-yl]oxy-propan-1-amine (720 mg, 596 μmol, 31% yield, 67% purity) as a yellow solid. ¹H NMR (400 MHz, CDCl₃) δ 7.83 (d, J=1.6 Hz, 1H), 7.79 (s, 1H), 7.04-6.96 (m, 1H), 6.11 (d, J=18.0 Hz, 1H), 5.89 (dd, J=2.4, 9.2 Hz, 1H), 5.56 (dd, J=0.8, 11.6 Hz, 1H), 4.50 (td, J=5.6, 11.2 Hz, 1H), 4.27-4.20 (m, 3H), 4.15-4.10 (m, 1H), 4.06 (d, J=11.2 Hz, 1H), 3.83 (t, J=5.2 Hz, 2H), 3.71 (d, J=2.0 Hz, 3H), 3.57-3.53 (m, 2H), 3.01 (s, 3H), 2.75-2.65 (m, 2H), 2.58-2.46 (m, 1H), 2.26-2.17 (m, 2H), 2.14-2.10 (m, 3H), 1.77 (t, J=8.8 Hz, 2H), 1.66 (s, 1H), 1.40 (dt, J=1.2, 7.2 Hz, 3H), 1.17 (dd, J=6.4, 7.2 Hz, 3H), 0.78 (s, 9H), −0.11 (d, J=3.6 Hz, 7H). LCMS: (M+1: 819.7).

Step 5. A mixture of (2S)-N-[[2-[2-[tert-butyl(dimethyl)silyl]oxyethyl]-5-ethoxy-4-iodo-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (669 mg, 816 μmol, 1 eq), Pd(OAc)₂ (36.6 mg, 163 mol, 0.2 eq), TBAC (227 mg, 816 mol, 1 eq), NaHCO₃ (171 mg, 2.04 mmol, 2.5 eq) in DMF (26 mL) was stirred at 120° C. for 16 h. On completion, the mixture was quenched with water (30 mL) and extracted with ethyl acetate (15 mL×3). The combined organic phase was dried over anhydrous sodium sulfate, filtered and concentrated to give a residue. The residue was purified by column chromatography (SiO₂, Petroleum ether/THF=1/0 to 2/1) to give (10S,17E)-14-(2-{[tert-butyl(dimethyl)silyl]oxy}ethyl)-16-ethoxy-8,10,12,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (295 mg, 306 μmol, 37% yield, 72% purity) as a yellow solid. LCMS: (M+1: 691.3).

Step 6. To a solution of (10S,17E)-14-(2-{[tert-butyl(dimethyl)silyl]oxy}ethyl)-16-ethoxy-8,10,12,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (245 mg, 354 μmol, 1 eq) in DCM (3 mL) was added TFA (4.61 g, 40.3 mmol, 3 mL, 114 eq). The mixture was stirred at 25° C. for 0.5 h. On completion, the mixture was concentrated to give a yellow oil (208 mg, 354 μmol, 100% yield). LCMS: (M+1: 589.2).

This crude yellow oil (208 mg, 353 μmol, 1 eq) was dissolved in MeOH (5 mL), and K₂CO₃ (244 mg, 1.77 mmol, 5 eq) was added. The mixture was stirred at 25° C. for 10 min. On completion, the mixture was filtered and concentrated in vacuum. The residue was purified by Prep-HPLC (column: Welch Ultimate C18 150*25 mm*5 um; mobile phase: [water(FA)-ACN]; gradient:9%-39% B over 10 min) to give Ex. 29 (76.28 mg, 141.63 μmol, 40.08% yield, FA salt) as a yellow solid. LCMS: (M+1: 493.2). ¹H NMR for Ex. 29 is shown in the below NMR table.

### Preparation of {[(11S,17E)-14-(2-hydroxyethyl)-6,8,11,12-tetramethyl-2,10,11,12,13,14-hexahydro-8H-5,3-(azenometheno)tripyrazolo|3,4-f:3′,4′-j:4″,3″-n][1,4]oxazacyclopentadecin-16-yl]oxy}acetonitrile (Ex. 31)

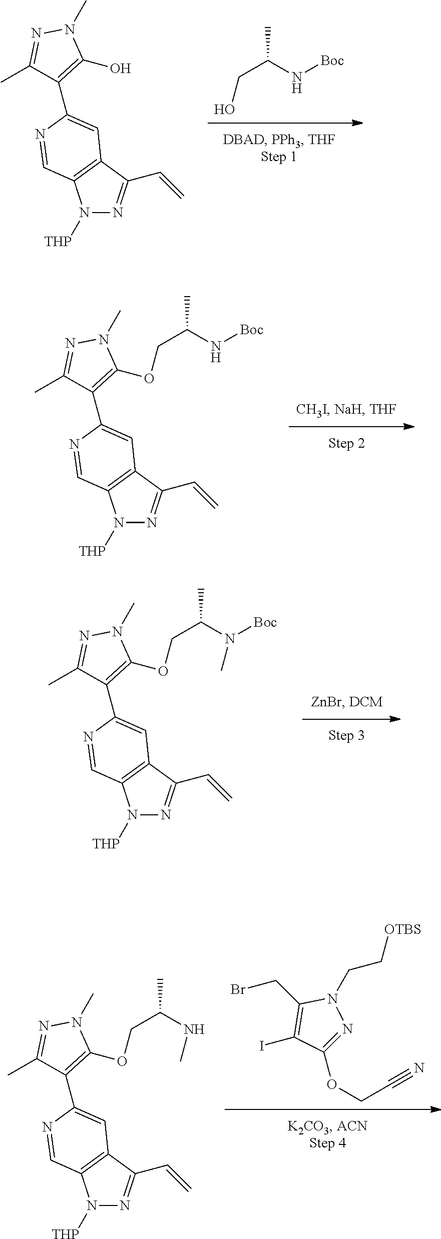

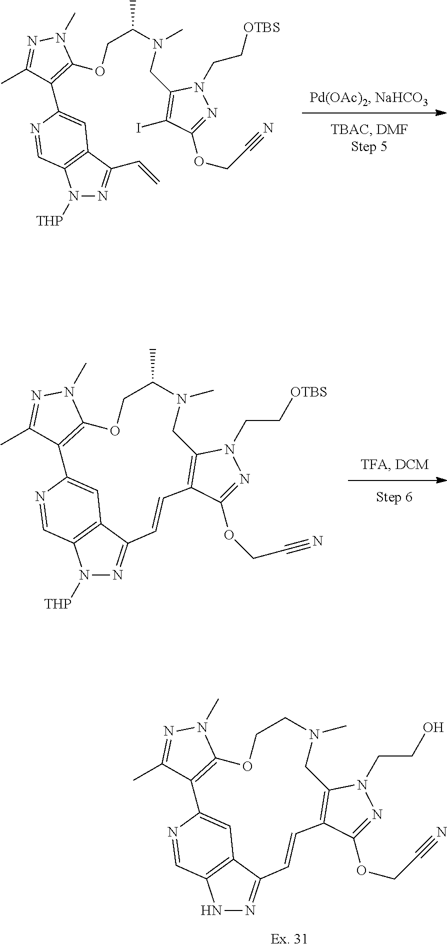

Step 1. A mixture of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (700 mg, 2.06 mmol, 1 eq), commercially available tert-butyl N-[(1S)-2-hydroxy-1-methyl-ethyl]carbamate (361 mg, 2.06 mmol, 1 eq), PPh₃ (1.08 g, 4.13 mmol, 2 eq) and DBAD (712 mg, 3.09 mmol, 1.5 eq) was degassed and purged with N₂ for 3 times and then THE (10.5 mL) was added to the mixture. The mixture was stirred at 25° C. for 1 h. On completion, the mixture was concentrated to give a residue. The residue was purified by column chromatography (SiO₂,PE:THF=1:0 to 60:40) to give tert-butyl N-[(1S)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]carbamate (880 mg, 1.77 mmol, 85% yield) as a yellow solid. LCMS: (M+1:497.1).

Step 2. To a solution of tert-butyl N-[(1S)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]carbamate (830 mg, 1.67 mmol, 1 eq) in THE (8.5 mL) was added NaH (133 mg, 3.34 mmol, 60% purity, 2 eq) at 0° C., the mixture was stirred at 0° C. for 0.5 h, then CH₃I (355 mg, 2.51 mmol, 1.5 eq) was added to the mixture. The mixture was stirred at 25° C. for 2 h. On completion, the mixture was quenched with sat. NH₄Cl (20 mL) and extracted with ethyl acetate (25 mL×3). The combined organic phase was dried over anhydrous sodium sulfate, filtered and concentrated to give tert-butyl N-[(1S)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]-N-methyl-carbamate (1.05 g, crude) as a yellow oil. LCMS: (M+1:511.2).

Step 3. To a solution of tert-butyl N-[(1S)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]-N-methyl-carbamate (1.00 g, 1.96 mmol, 1 eq) in DCM (10 mL) was added ZnBr₂ (2.21 g, 9.79 mmol, 5 eq). The mixture was stirred at 25° C. for 12 h. On completion, the mixture was quenched with water (50 mL) and extracted with DCM (40 mL×3). The combined organic phase was dried over anhydrous sodium sulfate, filtered and concentrated to give a residue. The residue was purified by column chromatography (SiO₂, Dichloromethane:Methanol=1:0 to 5:1) to give (2S)-1-[2,5-dimethyl-4-(1-tetrahydropyran-2-yl-3-vinyl-pyrazolo[3,4-c]pyridin-5-yl)pyrazol-3-yl]oxy-N-methyl-propan-2-amine (380 mg, 925 μmol, 47% yield) as yellow solid. LCMS: (M+1:411.1).

Step 4. A mixture of (2S)-1-[2,5-dimethyl-4-(1-tetrahydropyran-2-yl-3-vinyl-pyrazolo[3,4-c]pyridin-5-yl)pyrazol-3-yl]oxy-N-methyl-propan-2-amine (350 mg, 852 mol, 1 eq), 2-[5-(bromomethyl)-1-[2-[tert-butyl(dimethyl)silyl]oxyethyl]-4-iodo-pyrazol-3-yl]oxyacetonitrile, which was prepared according to the method described in Ex. 22, (426 mg, 852 mol, 1 eq), K₂CO₃ (353 mg, 2.56 mmol, 3 eq) in ACN (5 mL) was stirred at 60° C. for 2 h. On completion, the mixture was filtered and concentrated to give 2-[1-[2-[tert-butyl(dimethyl)silyl]oxyethyl]-5-[[[[(1S)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]-methyl-amino]methyl]-4-iodo-pyrazol-3-yl]oxyacetonitrile (711 mg, crude) as a yellow oil. LCMS: (M+1:830.4).

Step 5. A mixture of 2-[1-[2-[tert-butyl(dimethyl)silyl]oxyethyl]-5-[[[(1S)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]-methyl-amino]methyl]-4-iodo-pyrazol-3-yl]oxyacetonitrile (520 mg, 626 mol, 1 eq), NaHCO₃ (131 mg, 1.57 mmol, 2.5 eq), TBAC (174 mg, 626 μmol, 1 eq) and Pd(OAc)₂ (28.1 mg, 125 μmol, 0.2 eq) in DMF (26 mL) was degassed and purged with N₂ for 3 times, and then the mixture was stirred at 130° C. for 2 h under N₂ atmosphere. On completion, the mixture was quenched with water (50 mL) and extracted with ethyl acetate (30 mL×2). The combined organic phase was dried over anhydrous sodium sulfate, filtered, and concentrated to give a residue. The residue was purified by column chromatography (SiO₂, PE:THF=1:0 to 1:1) to give {[(11S,17E)-14-(2-{[tert-butyl(dimethyl)silyl]oxy}ethyl)-6,8,11,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (320 mg, 455 μmol, 72% yield) as yellow solid. LCMS: (M+1:702.5).

Step 6. To a solution of 2-[[(9S,17E)-13-[2-[tert-butyl(dimethyl)silyl]oxyethyl]-3,5,9,10-tetramethyl-21-tetrahydropyran-2-yl-7-oxa-4,5,10,13,14,20,21,24-octazapentacyclo [17.5.2.0^{2.6}.0^{12,16}.0^{22,26}]hexacosa-1(25),2(6),3,12(16),14,17,19,22(26),23-nonaen-15-yl]oxy]acetonitrile (290 mg, 413 mol, 1 eq) in DCM (5 mL) was added TFA (13.4 mmol, 1 mL, 32.5 eq). The mixture was stirred at 25° C. for 1 h. On completion, the mixture was concentrated to give a residue. The residue was purified by prep-HPLC (column: Waters xbridge 150*25 mm 10 um; mobile phase: [water (NH4HCO3)-ACN]; gradient: 20%-50% B over 14 min) to give Ex. 31 (17.04 mg, 32.18 mol, 7.79% yield, 95.1% purity) as a yellow solid. LCMS: (M+1:504.1). ¹H NMR for Ex. 31 is shown in the below NMR table.

### Preparation of (2S)-2-[(10R,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 35)

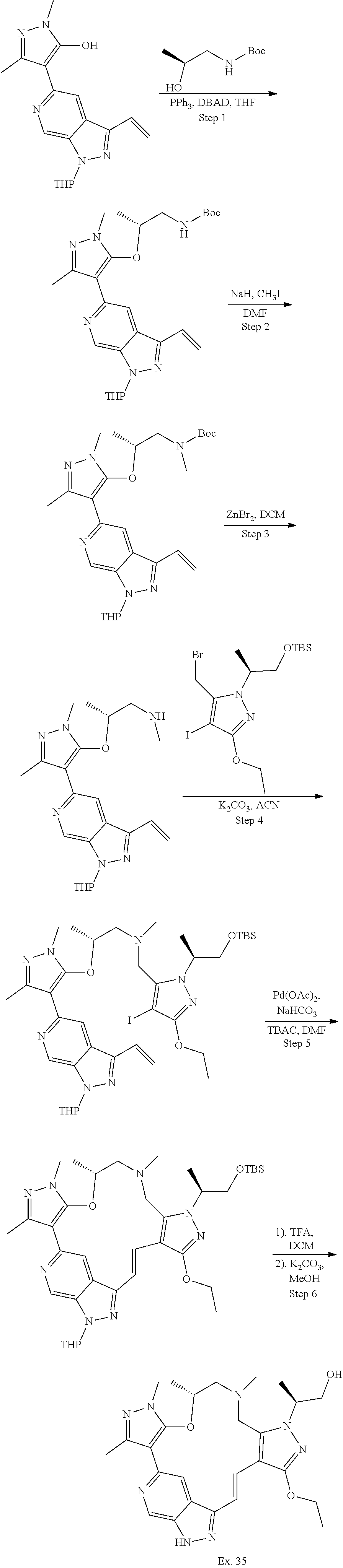

Step 1. A mixture of commercially available tert-butyl N-[(2S)-2-hydroxypropyl]carbamate (470 mg, 2.68 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (1.00 g, 2.95 mmol, 1.1 eq), PPh₃ (1.41 g, 5.36 mmol, 2 eq) and DBAD (926 mg, 4.02 mmol, 1.5 eq) was degassed and purged with N₂ for 3 times, and then THE (10 mL) was added. The mixture was stirred at 25° C. for 1 h. On completion, the mixture was concentrated to give a residue. The residue was purified by column chromatography (SiO₂,PE:THF=1:0 to 1:1) to give tert-butyl N-[(2R)-2-[2,5-dimethyl-4-(1-tetrahydropyran-2-yl-3-vinyl-pyrazolo[3,4-c]pyridin-5-yl)pyrazol-3-yl]oxypropyl]carbamate (1.33 g, 2.68 mmol, 100% yield) as a yellow oil. LCMS: (M+1:497.6).

Step 2. To a solution of tert-butyl N-[(2R)-2-[2,5-dimethyl-4-(1-tetrahydropyran-2-yl-3-vinyl-pyrazolo[3,4-c]pyridin-5-yl)pyrazol-3-yl]oxypropyl]carbamate (1.30 g, 2.62 mmol, 1 eq) in DMF (13 mL) was added NaH (262 mg, 6.54 mmol, 60% purity, 2.5 eq) and was stirred at 25° C. for 0.5 h. Then CH₃I (356 mg, 2.51 mmol, 1.5 eq) was added at 0° C. The mixture was stirred at 25° C. for 1 h. On completion, the mixture was quenched with NH₄Cl (26 mL) and extracted with ethyl acetate (25 mL×3). The combined organic phase was dried over anhydrous sodium sulfate, filtered and concentrated to give tert-butyl N-[(2R)-2-[2,5-dimethyl-4-(1-tetrahydropyran-2-yl-3-vinyl-pyrazolo[3,4-c]pyridin-5-yl)pyrazol-3-yl]oxypropyl]-N-methyl-carbamate (1.30 g, 2.55 mmol, 97% yield) as a yellow oil. LCMS: (M+1:511.1).

Step 3. To a solution of tert-butyl N-[(2R)-2-[2,5-dimethyl-4-(1-tetrahydropyran-2-yl-3-vinyl-pyrazolo[3,4-c]pyridin-5-yl)pyrazol-3-yl]oxypropyl]-N-methyl-carbamate (1.30 g, 2.55 mmol, 1 eq) in DCM (13 mL) was added ZnBr₂ (2.87 g, 12.7 mmol, 5 eq). The mixture was stirred at 25° C. for 12 h. On completion, the mixture was quenched with water (16 mL) and extracted with DCM (15 mL×3). The combined organic phase was dried over anhydrous sodium sulfate, filtered and concentrated to give a residue. The residue was purified by column chromatography (SiO₂, DCM/MeOH=1:0 to 10:1) to give (2R)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (612 mg, 1.49 mmol, 59% yield) as a white solid. LCMS: (M+1:411.3).

Step 4. To a solution of (2R)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (612 mg, 1.49 mmol, 1 eq) and [(2S)-2-[5-(bromomethyl)-3-ethoxy-4-iodo-pyrazol-1-yl]propoxy]-tert-butyl-dimethyl-silane, which was prepared according to the method described in Ex. 19, (900 mg, 1.79 mmol, 1.2 eq) in ACN (7 mL) was added K₂CO₃ (618 mg, 4.47 mmol, 3 eq). The mixture was stirred at 60° C. for 12 h. On completion, the mixture was filtered and concentrated to give a residue. The residue was purified by column chromatography (SiO₂, PE/THF=1:0 to 2:1) to give (2R)-N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (615 mg, 738 Pmol, 50% yield) as a yellow oil. LCMS: (M+1:833.3).

Step 5. A mixture of (2R)-N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (615 mg, 738 μmol, 1 eq) in DMF (31 mL) was added TBAC (205 mg, 738 mol, 1 eq) and NaHCO₃ (155 mg, 1.85 mmol, 2.5 eq) and Pd(OAc)₂ (33.2 mg, 148 mol, 0.2 eq) degassed and purged with N₂ for 3 times, and then the mixture was stirred at 140° C. for 1 h under N₂ atmosphere. On completion, the mixture was quenched with water (62 mL) and extracted with ethyl acetate (25 mL×3). The combined organic phase was dried over anhydrous sodium sulfate, filtered, and concentrated to give a residue. The residue was purified by column chromatography (SiO₂, PE/EA=1:0 to 1:1) to give (10R,17E)-14-[(2S)-1-{[tert-butyl(dimethyl)silyl]oxy}propan-2-yl]-16-ethoxy-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (314 mg, 445 μmol, 60% yield) as a yellow solid. LCMS: (M+1:705.4).

Step 6. To a solution of (10R,17E)-14-[(2S)-1-{[tert-butyl(dimethyl)silyl]oxy}propan-2-yl]-16-ethoxy-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (314 mg, 445 μmol, 1 eq) in DCM (3 mL) was added TFA (40.4 mmol, 3 mL, 90.7 eq). The mixture was stirred at 25° C. for 12 h. On completion, the mixture was concentrated to give a yellow oil (268 mg, 445 mol, 100% yield). LCMS: (M+1:603.2).

This yellow oil obtained (268 mg, 445 μmol, 1 eq) was dissolved in MeOH (5 mL), and K₂CO₃ (615 mg, 4.45 mmol, 10 eq) was added. The mixture was stirred at 25° C. for 0.5 h. On completion, the mixture was filtered and concentrated to give a residue. The crude product was purified by reversed-phase column chromatography (column: Phenomenex luna C18 150*25 mm*10 um; mobile phase: water(FA)-ACN; gradient:10%-40% B over 10 min) to give Ex. 35 (90.91 mg, 179.45 mol, 40.35% yield) as a yellow solid. LCMS: (M+1: 507.3). ¹H NMR for Ex. 35 is shown in the below NMR table.

### Preparation of 2-[(10R,17E)-16-ethoxy-8,10,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 38)

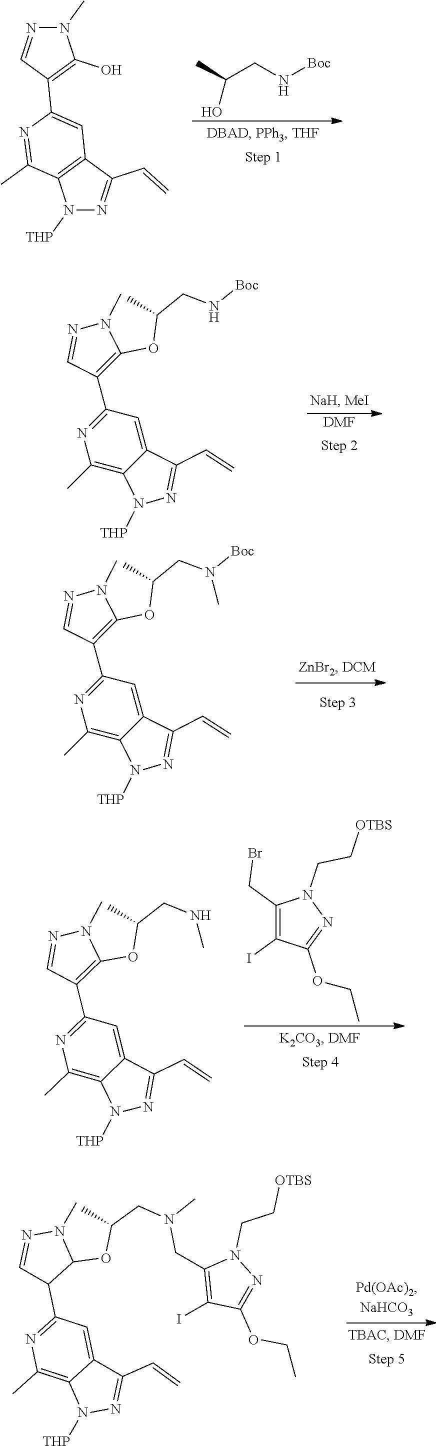

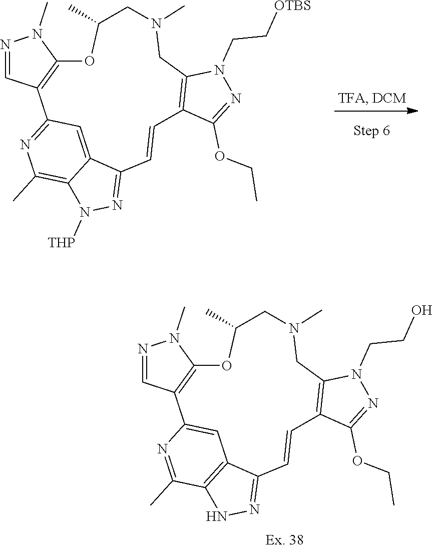

Step 1. A mixture of 2-methyl-4-(7-methyl-1-tetrahydropyran-2-yl-3-vinyl-pyrazolo[3,4-c]pyridin-5-yl)pyrazol-3-ol, which was prepared according to the method described in Ex. 28, (750 mg, 2.21 mmol, 1 eq), commercially available tert-butyl N-[(2S)-2-hydroxypropyl]carbamate (774 mg, 4.42 mmol, 2 eq), DBAD (1.12 g, 4.86 mmol, 2.2 eq), PPh₃ (1.28 g, 4.86 mmol, 2.2 eq) was degassed and purged with N₂ for 3 times. Then 2-methyltetrahydrofuran (10 mL) was added and purged with N₂ for 3 times again, and then the mixture was stirred at 25° C. for 2 h under N₂ atmosphere. On completion, the mixture was concentrated in vacuum to give a residue. The residue was purified by column chromatography (SiO₂, Petroleum ether/THF=2:1 to 1:1) to give tert-butyl N-[(2R)-2-[2-methyl-4-(7-methyl-1-tetrahydropyran-2-yl-3-vinyl-pyrazolo[3,4-c]pyridin-5-yl)pyrazol-3-yl]oxypropyl]carbamate (2.50 g, 2.01 mmol, 91% yield, 40% purity) as yellow oil. LCMS: (M+1: 497.1).

Step 2. To a solution of tert-butyl N-[(2R)-2-[2-methyl-4-(7-methyl-1-tetrahydropyran-2-yl-3-vinyl-pyrazolo [3,4-c]pyridin-5-yl)pyrazol-3-yl]oxypropyl]carbamate (2.40 g, 1.93 mmol, 1 eq) in DMF (10 mL) was added NaH (231 mg, 5.80 mmol, 60% purity, 3 eq) at 0° C. for 0.5 h. The mixture was added MeI (548 mg, 3.87 mmol, 240 μL, 2 eq) and stirred at 25° C. for 1.5 h. On completion, the mixture was quenched with sat. NH₄Cl (100 mL) and was extracted with EA (50 mL×3). The combined organic phase was dried over anhydrous sodium sulfate, filtered and concentrated to give tert-butyl N-methyl-N-[(2R)-2-[2-methyl-4-(7-methyl-1-tetrahydropyran-2-yl-3-vinyl-pyrazolo[3,4-c]pyridin-5-yl)pyrazol-3-yl]oxypropyl]carbamate (2.10 g, 1.85 mmol, 95% yield, 45% purity) as yellow solid. LCMS: (M+1: 511.1).

Step 3. To a solution of tert-butyl N-methyl-N-[(2R)-2-[2-methyl-4-(7-methyl-1-tetrahydropyran-2-yl-3-vinyl-pyrazolo[3,4-c]pyridin-5-yl)pyrazol-3-yl]oxypropyl]carbamate (2.00 g, 1.76 mmol, 1 eq) in DCM (20 mL) was added ZnBr₂ (1.19 g, 5.29 mmol, 3 eq). The mixture was stirred at 25° C. for 5 hr. On completion, the mixture was concentrated in vacuum. The residue was purified by column chromatography (SiO₂, DCM/MeOH=15:1 to 8:1) to give (2R)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (250 mg, 609 mol, 34% yield) as yellow solid. LCMS: (M+1: 411.1).

Step 4. To a solution of (2R)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (220 mg, 535 μmol, 1 eq), 2-[5-(bromomethyl)-3-ethoxy-4-iodo-pyrazol-1-yl]ethoxy-tert-butyl-dimethyl-silane, prepared according to the method described in Ex. 1, (393 mg, 803 mol, 1.5 eq) in DMF (2 mL) was added K₂CO₃ (222 mg, 1.61 mmol, 3 eq). The mixture was stirred at 80° C. for 2 hr. On completion, the mixture was poured into H₂O (30 mL) and extracted with EA (15 mL×3). The combined organic phase was washed with H₂O (20 mL×3) and dried over anhydrous sodium sulfate, filtered and concentrated to give a residue. The residue was purified by column chromatography (SiO₂, Petroleum ether/THF=1:1 to 1:2) to give (2R)-N-[[2-[2-[tert-butyl(dimethyl)silyl]oxyethyl]-5-ethoxy-4-iodo-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (140 mg, 170 mol, 31% yield) as yellow solid. LCMS: (M+1: 819.1).

Step 5. A mixture of (2R)-N-[[2-[2-[tert-butyl(dimethyl)silyl]oxyethyl]-5-ethoxy-4-iodo-pyrazol-3-yl]methyl]-N-methyl-2-|2-methyl-4-(7-methyl-1-tetrahydropyran-2-yl-3-vinyl-pyrazolo[3,4-c]pyridin-5-yl)pyrazol-3-yl]oxy-propan-1-amine (120 mg, 146 μmol, 1 eq), diacetoxypalladium (6.58 mg, 29.3 μmol, 0.2 eq), TBAC (40.7 mg, 146 μmol, 1 eq) and sodium bicarbonate (30.7 mg, 366 mol, 2.5 eq) in DMF (2 mL) was degassed and purged with N₂ for 3 times, and then the mixture was stirred at 130° C. for 1 h under N₂ atmosphere. On completion, the mixture was quenched with water (30 mL) and extracted with ethyl acetate (15 mL×3). The combined organic phase was dried over anhydrous sodium sulfate, filtered and concentrated to give a residue. The residue was purified by column chromatography (SiO₂, Petroleum ether/THF=1:1 to 1:3) to give (10R,17E)-14-(2-{[tert-butyl(dimethyl)silyl]oxy}ethyl)-16-ethoxy-8,10,12,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (100 mg, 115 μmol, 79% yield, 80% purity) as yellow solid. LCMS: (M+1: 691.2).

Step 6. To a solution of (10R,17E)-14-(2-{[tert-butyl(dimethyl)silyl]oxy}ethyl)-16-ethoxy-8,10,12,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (80.0 mg, 115 μmol, 1 eq) in DCM (3 mL) was added TFA (921 mg, 8.08 mmol, 0.6 mL, 69 eq). The mixture was stirred at 25° C. for 1 hr. On completion, the mixture was concentrated in vacuum to give a residue. The residue was purified by prep-HPLC purification (column: Waters Xbridge C18 150*50 mm*10 um; mobile phase: [water(NH₃H₂O)-ACN]; gradient:17%-47% B over 11 min) to give Ex. 38 (15.3 mg, 30.4 mol, 26.2% yield, 97.4% purity) as off-white solid. LCMS: (M+1: 493.1). ¹H NMR for Ex. 38 is shown in the below NMR table.

### Preparation of 2-[(10S,17E)-8,10,12,20-tetramethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 40)

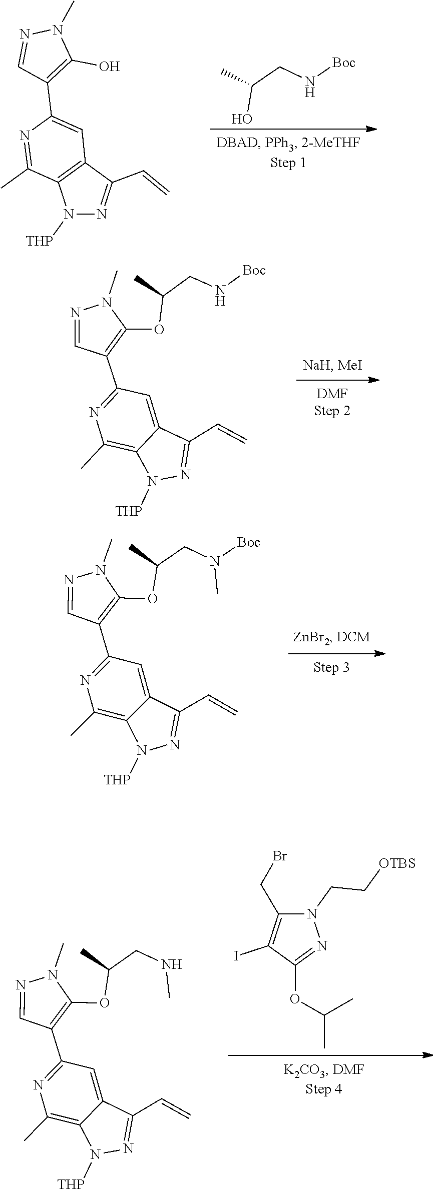

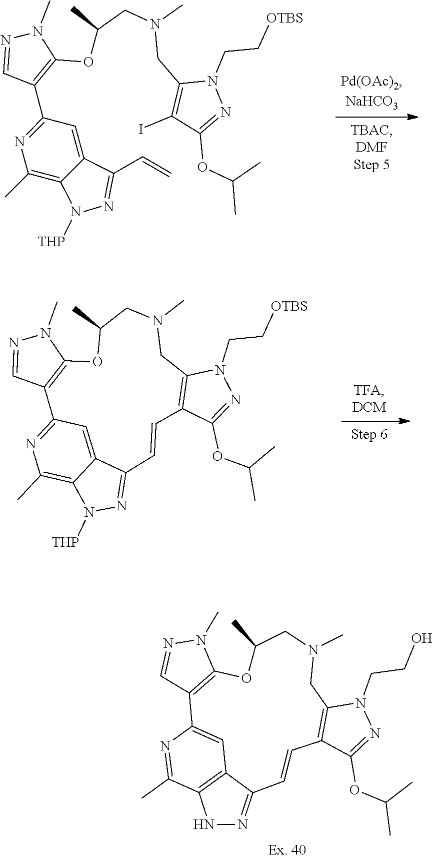

Step 1. A mixture of 2-methyl-4-(7-methyl-1-tetrahydropyran-2-yl-3-vinyl-pyrazolo[3,4-c]pyridin-5-yl)pyrazol-3-ol, which was prepared according to the method described in Ex. 28, (800 mg, 2.36 mmol, 1 eq), commercially available tert-butyl N-[(2R)-2-hydroxypropyl]carbamate (826 mg, 4.71 mmol, 2 eq), DBAD (1.19 g, 5.19 mmol, 2.2 eq), PPh₃ (1.36 g, 5.19 mmol, 2.2 eq) was degassed and purged with N₂ for 3 times, then added 2-methyltetrahydrofuran (10 mL) was degassed and purged with N₂ for 3 times again, and then the mixture was stirred at 25° C. for 2 h under N₂ atmosphere. On completion, the mixture was concentrated in vacuo to give a residue. The residue was purified by column chromatography (SiO₂, Petroleum ether/THF=2:1 to 1:1) to give tert-butyl N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (2.10 g, 2.20 mmol, 93% yield, 52% purity) as yellow solid. LCMS: (M+1: 497.1).

Step 2. To a solution of tert-butyl N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (2.00 g, 2.09 mmol, 1 eq) in DMF (20 mL) was added NaH (251 mg, 6.28 mmol, 60% purity, 3 eq) at 0° C. for 0.5 h. The mixture was added MeI (594 mg, 4.19 mmol, 260 μL, 2 eq) and stirred at 25° C. for 1.5 h. On completion, the mixture was quenched by sat. NH₄Cl (200 mL) and extracted with EA (100 mL×3). The combined organic phase was dried over anhydrous sodium sulfate, filtered and concentrated to give a residue. The residue was purified by column chromatography (SiO₂, Petroleum ether/THF=3:1 to 1:1) to give tert-butyl N-methyl-N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (680 mg, 1.33 mmol, 63% yield) as yellow oil. ¹H NMR (400 MHz, CDCl₃) δ=7.88-7.72 (m, 2H), 7.08-6.96 (m, 1H), 6.11 (d, J=18.0 Hz, 1H), 5.89 (br d, J=9.2 Hz, 1H), 5.58 (d, J=11.6 Hz, 1H), 4.74-4.47 (m, 1H), 4.06 (br d, J=10.4 Hz, 1H), 3.77-3.71 (m, 5H), 3.58 (br s, 2H), 3.01 (s, 3H), 2.96-2.84 (m, 3H), 2.77-2.64 (m, 1H), 2.26-2.13 (m, 2H), 1.52-1.41 (m, 6H), 1.40-1.34 (m, 4H), 1.16 (br d, J=5.2 Hz, 3H), 0.93-0.82 (m, 1H). LCMS: (M+1: 511.2).

Step 3. To a solution of tert-butyl N-methyl-N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]carbamate (350 mg, 685 mol, 1 eq) in DCM (5 mL) was added ZnBr₂ (463 mg, 2.06 mmol, 3 eq). The mixture was stirred at 25° C. for 1 hr. On completion, the mixture was concentrated in vacuum to give (2S)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (850 mg, 621 μmol, 90% yield, 30% purity) as yellow solid. LCMS: (M+1: 411.1).

Step 4. To a solution of (2S)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (750 mg, 548 μmol, 1 eq), 2-[5-(bromomethyl)-4-iodo-3-isopropoxy-pyrazol-1-yl]ethoxy-tert-butyl-dimethyl-silane, which was prepared according to the method described in Ex. 3/Ex. 4, (413 mg, 822 μmol, 1.5 eq) in DMF (10 mL) was added K₂CO₃ (227 mg, 1.64 mmol, 3 eq). The mixture was stirred at 80° C. for 1 hr. On completion, the mixture was poured into H₂O (100 mL) and extracted with EA (50 mL×3). The combined organic phase was washed with H₂O (100 mL×3) and dried over anhydrous sodium sulfate, filtered and concentrated to give a residue. The residue was purified by column chromatography (SiO₂, Petroleum ether/THF=1:1 to 1:2) to give(2S)-N-[[2-[2-[tert-butyl(dimethyl)silyl]oxyethyl]-4-iodo-5-isopropoxy-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (350 mg, 420 mol, 76% yield) as colourless oil. LCMS: (M+1: 833.5).

Step 5. A mixture of (2S)-N-[[2-[2-[tert-butyl(dimethyl)silyl]oxyethyl]-4-iodo-5-isopropoxy-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (330 mg, 396 mol, 1 eq), diacetoxypalladium (17.7 mg, 79.2 mol, 0.2 eq), TBAC(110 mg, 396 μmol, 1 eq) and sodiumbicarbonate (83.2 mg, 990 mol, 2.5 eq) in DMF (6 mL) was degassed and purged with N₂ for 3 times, and then the mixture was stirred at 130° C. for 1 h under N₂ atmosphere. On completion, the mixture was poured into H₂O (100 mL) and extracted with EA (50 mL×3). The combined organic phase was washed with H₂O (100 mL×3) and dried over anhydrous sodium sulfate, filtered and concentrated to give a residue. The residue was purified by column chromatography (SiO₂, Petroleum ether/THF=1:1 to 0:1) to give (10S,17E)-14-(2-{[tert-butyl(dimethyl)silyl]oxy}ethyl)-8,10,12,20-tetramethyl-2-(oxan-2-yl)-16-[(propan-2-yl)oxy]-2,10,11,12,13,14-hexahydro-8H-5,3-(azenometheno)tripyrazolo[3,4-f:3′,4′-j:4″,3″-n][1,4]oxazacyclopentadecine (200 mg, 212 μmol, 53% yield, 75% purity) as yellow solid. LCMS: (M+1: 705.2).

Step 6. To a solution of (10S,17E)-14-(2-{[tert-butyl(dimethyl)silyl]oxy}ethyl)-8,10,12,20-tetramethyl-2-(oxan-2-yl)-16-[(propan-2-yl)oxy]-2,10,11,12,13,14-hexahydro-8H-5,3-(azenometheno)tripyrazolo[3,4-f:3′,4′-j:4″,3″-n][1,4]oxazacyclopentadecine (180 mg, 255 μmol, 1 eq) in DCM (0.5 mL) was added TFA (1.38 g, 12.1 mmol, 900 μL, 47 eq). The mixture was stirred at 25° C. for 1 hr. On completion, the mixture was concentrated in vacuo to give a residue. The residue was purified by prep-HPLC purification (column: Phenomenex luna C18 150*25 mm*10 um; mobile phase: [water(FA)-ACN]; gradient:10%-40% B over 10 min) to give Ex. 40 (33.5 mg, 65.3 mol, 25.6% yield, 98.8% purity) as yellow solid. LCMS: (M+1: 507.2). ¹H NMR for Ex. 40 is shown in the below NMR table.

### Preparation of 2-[(10S,17E)-6,8,10,12,16-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 41)

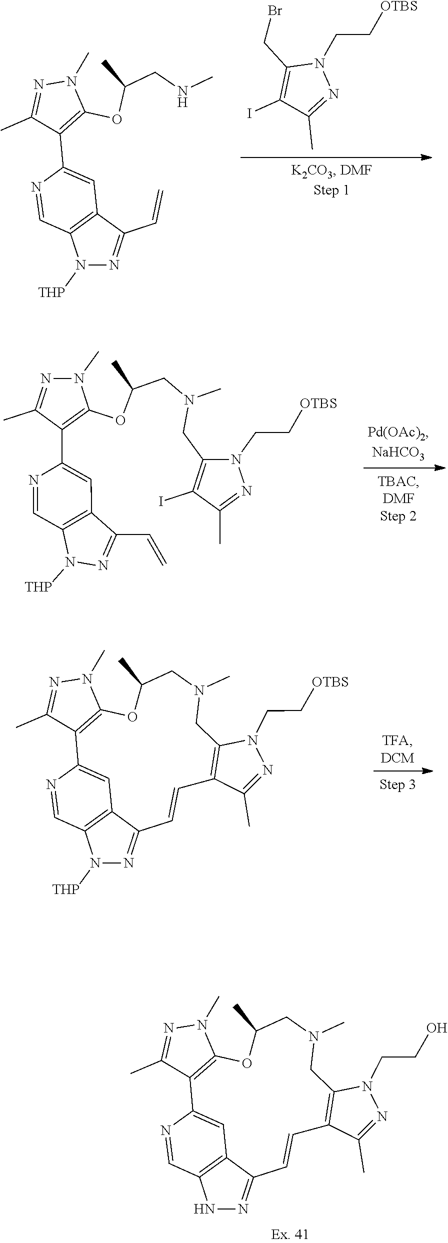

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (190 mg, 462 μmol, 1 eq), 2-[5-(bromomethyl)-4-iodo-3-methyl-pyrazol-1-yl]ethoxy-tert-butyl-dimethyl-silane, which was prepared according to the method described in Ex. 13 and 14, (255 mg, 555 mol, 1.2 eq) in DMF (2 mL) was added K₂CO₃ (191 mg, 1.39 mmol, 3 eq). The mixture was stirred at 25° C. for 14 hours. On completion, the reaction mixture was partitioned between ethyl acetate (10 mL×3) and water (10 mL). The combined organic phase was dried over anhydrous sodium sulfate, filtered and concentrated to give (2S)-N-[[2-[2-[tert-butyl(dimethyl)silyl]oxyethyl]-4-iodo-5-methyl-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (190 mg, 240 mol, 52% yield) as yellow oil. LCMS: (M+1: 789.1).

Step 2. A mixture of (2S)-N-[[2-[2-[tert-butyl(dimethyl)silyl]oxyethyl]-4-iodo-5-methyl-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (180 mg, 228 mol, 1 eq), Pd(OAc)₂ (10.2 mg, 45.6 mol, 0.2 eq), NaHCO₃ (47.9 mg, 570 μmol, 2.5 eq) and TBAC (63.4 mg, 228 μmol, 1 eq) in DMF (1 mL) was degassed and purged with N₂ for 3 times, and then the mixture was stirred at 120° C. for 0.5 hours under N₂ atmosphere. On completion, the reaction mixture was partitioned between ethyl acetate (5 mL×3) and water (5 mL). The combined organic phase was dried over anhydrous sodium sulfate, filtered and concentrated to give (10S,17E)-14-(2-{[tert-butyl(dimethyl)silyl]oxy}ethyl)-6,8,10,12,16-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (120 mg, 181 μmol, 79% yield) as yellow oil. LCMS: (M+1: 661.6).

Step 3. To a solution of (10S,17E)-14-(2-{[tert-butyl(dimethyl)silyl]oxy}ethyl)-6,8,10,12,16-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (100 mg, 151 μmol, 1 eq) in DCM (1 mL) was added TFA (17.2 mg, 151 gmol, 1 eq). The mixture was stirred at 25° C. for 2 hours. On completion, the reaction mixture was concentrated to give the residue. The residue was purified by prep-HPLC (column: Phenomenex luna C18 150*25 mm*10 um; mobile phase: [water(FA)-ACN]; gradient:0%-30% B over 10 mins) to give Ex. 41(19.2 mg, 37.7 μmol, 24% yield, 100% purity, FA) as white solid. LCMS: (M+1:463.1). ¹H NMR for Ex. 41 is shown in the below NMR table.

### Preparation of {[(10R,17E)-14-(2-hydroxyethyl)-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 42)

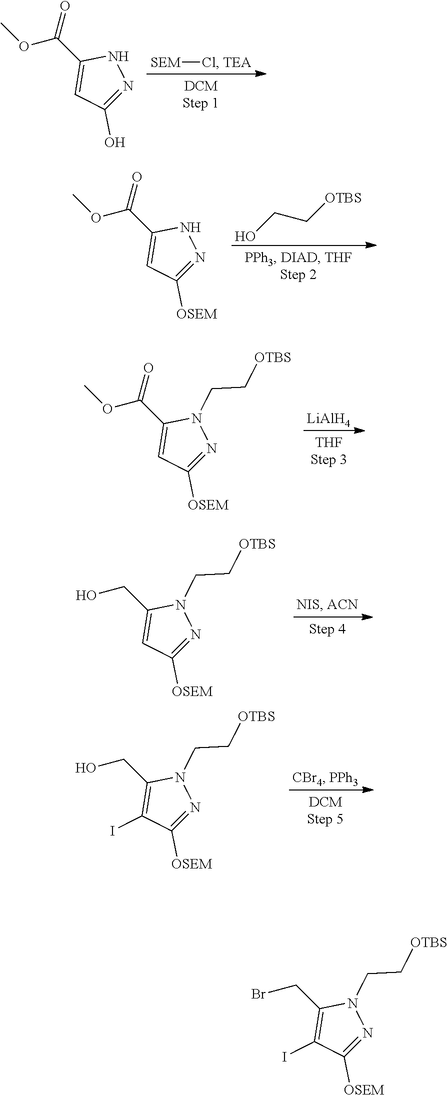

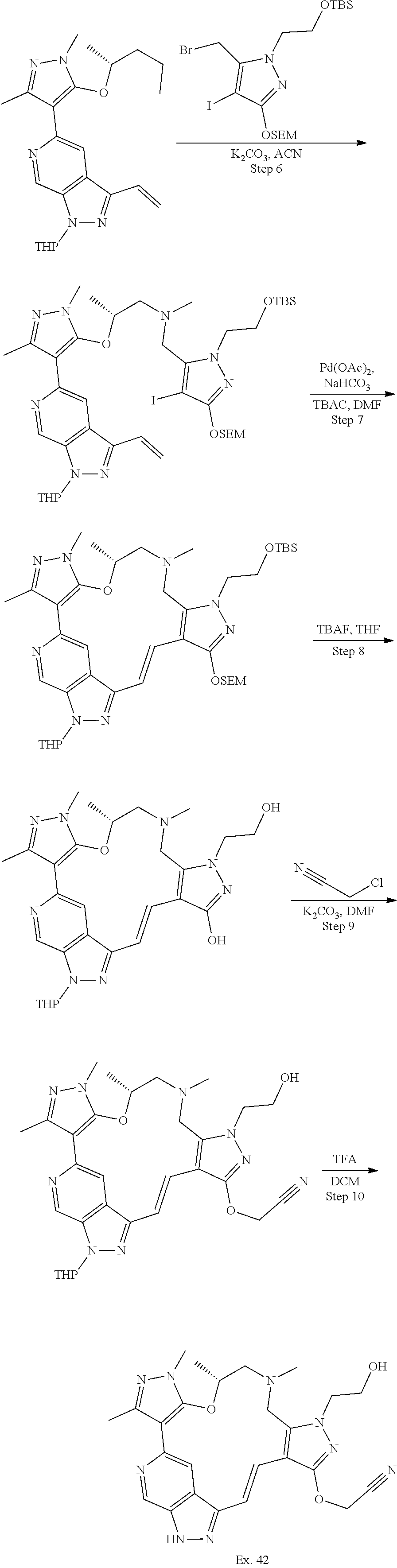

Step 1. To a mixture of commercially available methyl 3-hydroxy-1H-pyrazole-5-carboxylate (20.0 g, 140 mmol, 1.00 eq), TEA (85.0 g, 844 mmol, 117 mL, 6.00 eq) in DCM (200 mL) was added SEM-C1 (70.3 g, 422 mmol, 3.00 eq) at 0° C. and then the mixture was stirred at 25° C. for 2 h. On completion, the mixture was quenched with water (200 mL) and extracted with DCM (55 mL×3). The combined organic phase was dried over anhydrous sodium sulfate, filtered and concentrated to give a residue. The residue was purified by column chromatography (SiO₂, Petroleum ether/Ethyl acetate=1:0 to 0:1) to give methyl 3-(2-trimethylsilylethoxymethoxy)-1H-pyrazole-5-carboxylate (11.1 g, 29% yield) as pink solid. ¹H NMR (400 MHz, CDCl₃) δ=6.32-6.30 (m, 1H), 5.28-5.25 (m, 2H), 3.88-3.86 (m, 3H), 3.78-3.73 (m, 2H), 1.25-1.18 (m, 2H), 0.96-0.92 (m, 2H), −0.03-0.04 (m, 9H). LCMS: (M+1=273.2).

Step 2. A mixture of methyl 3-(2-trimethylsilylethoxymethoxy)-1H-pyrazole-5-carboxylate (10.0 g, 36.7 mmol, 1.00 eq), commercially available 2-[tert-butyl(dimethyl)silyl]oxyethanol (14.2 g, 80.7 mmol, 2.20 eq), and PPh₃ (21.1 g, 80.7 mmol, 2.20 eq) in THF (100 mL) was degassed and purged with N₂ for 3 times, and then DIAD (16.3 g, 80.7 mmol, 2.20 eq) was added at 0° C. The mixture was stirred at 25° C. for 2 h under N₂ atmosphere. On completion, the mixture was quenched with water (150 mL) and extracted with ethyl acetate (100 mL×3). The combined organic phase was dried over anhydrous sodium sulfate, filtered, and concentrated to give a residue. The residue was purified by column chromatography (SiO₂, Petroleum ether/Ethyl acetate=1:0 to 4:1) to give methyl 1-(2-((tert-butyldimethylsilyl)oxy)ethyl)-3-((2-(trimethylsilyl)ethoxy)methoxy)-1H-pyrazole-5-carboxylate (10.0 g, 23.2 mmol, 63.0% yield) as yellow oil. LCMS: (M+1=431.6).

Step 3. To a solution of methyl 1-(2-((tert-butyldimethylsilyl)oxy)ethyl)-3-((2-(trimethylsilyl)ethoxy)methoxy)-1H-pyrazole-5-carboxylate (11.5 g, 26.7 mmol, 1.00 eq) in THE (110 mL) was added LiAlH₄ (1.22 g, 32.0 mmol, 1.20 eq) at 0° C. The mixture was stirred at 25° C. for 1 h. On completion, MeOH (150 mL) was added to the mixture, and then the mixture was diluted with H₂O (150 mL) and extracted with EA (100 mL×3). The combined organic layers were dried over Na₂SO₄, filtered, and concentrated under reduced pressure. The residue was purified by column chromatography (SiO₂, Petroleum ether/Ethyl acetate=1:0 to 4:1) to give (1-(2-((tert-butyldimethylsilyl)oxy)ethyl)-3-((2-(trimethylsilyl)ethoxy)methoxy)-1H-pyrazol-5-yl)methanol (6.6 g, 61% yield) as off-white oil. LCMS: (M+1=403.1).

Step 4. To a solution of (1-(2-((tert-butyldimethylsilyl)oxy)ethyl)-3-((2-(trimethylsilyl)ethoxy)methoxy)-1H-pyrazol-5-yl)methanol (6.50 g, 16.1 mmol, 1.00 eq) in ACN (60 mL) was added NIS (3.99 g, 17.7 mmol, 1.10 eq) at 0° C. The mixture was stirred at 25° C. for 1 h. On completion, the reaction mixture was quenched by addition of saturated solution of Na₂SO₃ (60 mL), diluted with water (100 mL) and then extracted with ethyl acetate (50 mL×3). The combined organic phase was dried over anhydrous sodium sulfate, filtered, and concentrated. The residue was purified by column chromatography (SiO₂, Petroleum ether/Ethyl acetate=1:0 to 4:1) to give (1-(2-((tert-butyldimethylsilyl)oxy)ethyl)-4-iodo-3-((2-(trimethylsilyl)ethoxy)methoxy)-1H-pyrazol-5-yl)methanol (6.60 g, 77% yield) as off-white oil. LCMS: (M+1=529.3).

Step 5. A mixture of (1-(2-((tert-butyldimethylsilyl)oxy)ethyl)-4-iodo-3-((2-(trimethylsilyl)ethoxy)methoxy)-1H-pyrazol-5-yl)methanol (6.00 g, 11.3 mmol, 1.00 eq) and PPh₃ (3.57 g, 13.6 mmol, 1.20 eq) in DCM (60 mL) was degassed and purged with N₂ for 3 times at 0° C., and then CBr₄ (4.52 g, 13.6 mmol, 1.20 eq) was added at 0° C. The mixture was stirred at 25° C. for 1 h under N₂ atmosphere. On completion, the mixture was quenched with water (60 mL) and extracted with DCM (50 mL×3). The combined organic phase was dried over anhydrous sodium sulfate, filtered and concentrated to give a residue. The residue was purified by column chromatography (SiO₂, Petroleum ether/Ethyl acetate=1:0 to 4:1) to give 5-(bromomethyl)-1-(2-((tert-butyldimethylsilyl)oxy)ethyl)-4-iodo-3-((2-(trimethylsilyl)ethoxy)methoxy)-1H-pyrazole (a.k.a 2-μ5-(bromomethyl)-1-|2-[tert-butyl(dimethyl)silyl]oxyethyl]-4-iodo-pyrazol-3-yl]oxymethoxy]ethyl-trimethyl-silane) (3.00 g, 5.07 mmol, 45% yield) as an off-white oil. LCMS: (M+1=592.9).

Step 6. To a solution of (2R)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 35, (340 mg, 828 μmol, 1 eq) in ACN (0.5 mL) was added 2-[[5-(bromomethyl)-1-[2-[tert-butyl(dimethyl)silyl]oxyethyl]-4-iodo-pyrazol-3-yl]oxymethoxy]ethyl-trimethyl-silane (539 mg, 911 mol, 1.1 eq) and K₂CO₃ (343 mg, 2.48 mmol, 3 eq). The mixture was stirred at 60° C. for 2 h. On completion, the mixture was concentrated to give a residue. The residue was purified by column chromatography (SiO₂, PE:THF=2:1 to 2:1) to give (2R)-N-[[2-[2-[tert-butyl(dimethyl)silyl]oxyethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (320 mg, 347 mol, 42% yield) as a white solid. LCMS: (M+1: 921.8).

Step 7. A mixture of (2R)-N-[[2-[2-[tert-butyl(dimethyl)silyl]oxyethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (290 mg, 315 mol, 1 eq) in DMF (14.5 mL) was added NaHCO₃ (66.1 mg, 787 mol, 2.5 eq), TBAC (87.5 mg, 315 μmol, 1 eq) and Pd(OAc)₂ (14.14 mg, 62.9 mol, 0.2 eq). The mixture was degassed and purged with N₂ for 3 times, and then the mixture was stirred at 130° C. for 0.5 h under N₂ atmosphere. On completion, the reaction mixture was partitioned between EA (45 mL×3) and water (45 mL). The combined organic phase was dried over anhydrous sodium sulfate, filtered and concentrated to give a residue. The residue was purified by column chromatography (SiO₂, PE:THF=1:1 to 1:1) to give (10R,17E)-14-(2-{[tert-butyl(dimethyl)silyl]oxy}ethyl)-6,8,10,12-tetramethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (243 mg, 306 μmol, 97% yield) as a yellow solid. LCMS: (M+1: 793.3).

Step 8. To a solution of (10R,17E)-14-(2-{[tert-butyl(dimethyl)silyl]oxy}ethyl)-6,8,10,12-tetramethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (223 mg, 281 mol, 1 eq) in THE (1.69 mL) was added TBAF (1 M, 1.69 mL, 6 eq). The mixture was stirred at 70° C. for 12 h. On completion, the reaction mixture was partitioned between EA (4 mL×3) and water (4 mL). The combined organic phase was dried over anhydrous sodium sulfate, filtered and concentrated to give a residue. The residue was purified by column chromatography (SiO₂, DCM:MeOH=15:1 to 15:1) to give (10R,17E)-14-(2-hydroxyethyl)-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (92.0 mg, 167 mol, 59% yield) as a white solid. LCMS: (M+1: 549.2).

Step 9. To a solution of (10R,17E)-14-(2-hydroxyethyl)-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (82.0 mg, 149 mol, 1 eq) in DMF (1 mL) was added K₂CO₃ (20.7 mg, 149 mol, 1 eq) and commercially available 2-iodoacetonitrile (24.9 mg, 149 μmol, 1 eq). The mixture was stirred at 80° C. for 2 h. On completion, the reaction mixture was partitioned between EA (3 mL×3) and water (3 mL). The combined organic phase was dried over anhydrous sodium sulfate, filtered and concentrated to give a residue. The residue was purified by column chromatography (SiO₂, DCM:MeOH=20:1 to 20:1) to give {[(10R,17E)-14-(2-hydroxyethyl)-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (67.0 mg, 114 mol, 76% yield) as a white solid. LCMS: (M+1: 588.2).

Step 10. To a solution of {[(10R,17E)-14-(2-hydroxyethyl)-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (57.0 mg, 97.0 μmol, 1 eq) in DCM (0.5 mL) was added TFA (11.0 mg, 97.0 μmol, 1 eq). The mixture was stirred at 25° C. for 4 h. On completion, the mixture was concentrated to give a residue. The residue was purified by reversed phase column (column: Waters xbridge 150*25 mm 10 um; mobile phase: [water(NH4HCO3)-ACN]; gradient:22%-52% B over 14 min) to give Ex. 42 (20.11 mg, 39.94 μmol, 41.17% yield) as a yellow solid. LCMS: (M+1: 504.2). ¹H NMR for Ex. 42 is shown in the below NMR table.

### Preparation of 2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 44)

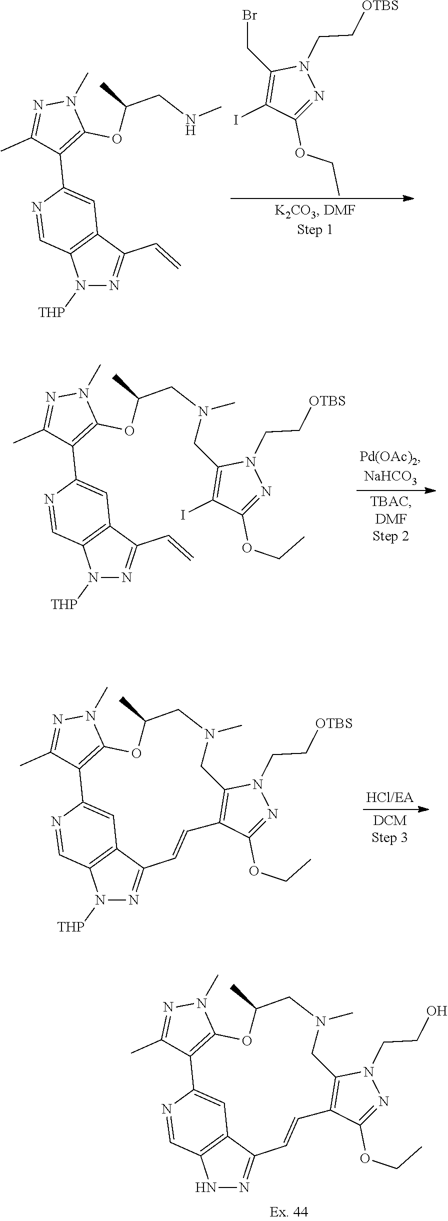

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (720 mg, 1.75 mmol, 1.00 eq) in DMF (6 mL) was added K₂CO₃(727 mg, 5.26 mmol, 3.00 eq) and 2-[5-(bromomethyl)-3-ethoxy-4-iodo-pyrazol-1-yl]ethoxy-tert-butyl-dimethyl-silane, which was prepared according to the method described in Ex. 1, (172 mg, 351 mol, 0.200 eq). The mixture was stirred at 80° C. for 2 hr. On completion, the reaction mixture was diluted with H₂O (15 mL) and extracted with EA (30 mL×3). The combined organic layers were dried over Na₂SO₄, filtered, and concentrated under reduced pressure to give a residue. The residue was purified by column chromatography (SiO₂, Petroleum ether/Ethyl acetate=1/0 to 0/1) to give (2S)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (400 mg, 488 mol, 28% yield) as a yellow oil. LCMS: (M+1=819.2).

Step 2. A mixture of (2S)-N-[[2-[2-[tert-butyl(dimethyl)silyl]oxyethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (360 mg, 440 μmol, 1.00 eq), Pd(OAc)₂ (19.7 mg, 87.9 μmol, 0.200 eq), NaHCO₃ (92.3 mg, 1.10 mmol, 2.50 eq) and TBAC (122 mg, 440 mol, 1.00 eq) in DMF (4 mL) was degassed and purged with N₂ for 3 times, and then the mixture was stirred at 140° C. for 1 hr under N₂ atmosphere. On completion, the residue was diluted with H₂O (10 mL) and extracted with EA (10 mL×3). The combined organic layers were dried over Na₂SO₄, filtered, and concentrated under reduced pressure to give a residue. The residue was purified by column chromatography (SiO₂, Petroleum ether/Ethyl acetate=1/0 to 0/1) to give (10S,17E)-14-(2-{[tert-butyl(dimethyl)silyl]oxy}ethyl)-16-ethoxy-6,8,10,12-tetramethyl-2-(oxan-2-yl)-2,10,11,12,13,14-hexahydro-8H-5,3-(azenometheno)tripyrazolo[3,4-f3′,4′-j:4″,3″-n][1,4]oxazacyclopentadecine (220 mg, 318 μmol, 72% yield) as a red solid. LCMS: (M+1=691.5).

Step 3. To a solution of (10S,17E)-14-(2-{[tert-butyl(dimethyl)silyl]oxy}ethyl)-16-ethoxy-6,8,10,12-tetramethyl-2-(oxan-2-yl)-2,10,11,12,13,14-hexahydro-8H-5,3-(azenometheno)tripyrazolo|3,4-f:3′,4′-j:4″,3″-n][1,4|oxazacyclopentadecine (148 mg, 214 μmol, 1.00 eq) in DCM (0.4 mL) was added HCl/EtOAc (4.00 M, 0.100 mL, 1.87 eq). The mixture was stirred at 25° C. for 2 hr. On completion, the reaction mixture was concentrated under reduced pressure to remove solvent. The residue was purified by prep-HPLC (column: Welch Ultimate C18 150*25 mm*5 um; mobile phase: [water(FA)-ACN]; gradient: 10%-40% B over 10 min) to give Ex. 44 (37.020 mg, 75.16 mol, 35.09% yield, 100% purity) as an off-white solid. LCMS: (M+1=493.2). ¹H NMR for Ex. 44 is shown in the below NMR table.

### Preparation of (2S)-2-[(10S,17E)-16-ethoxy-8,10,12,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 45)

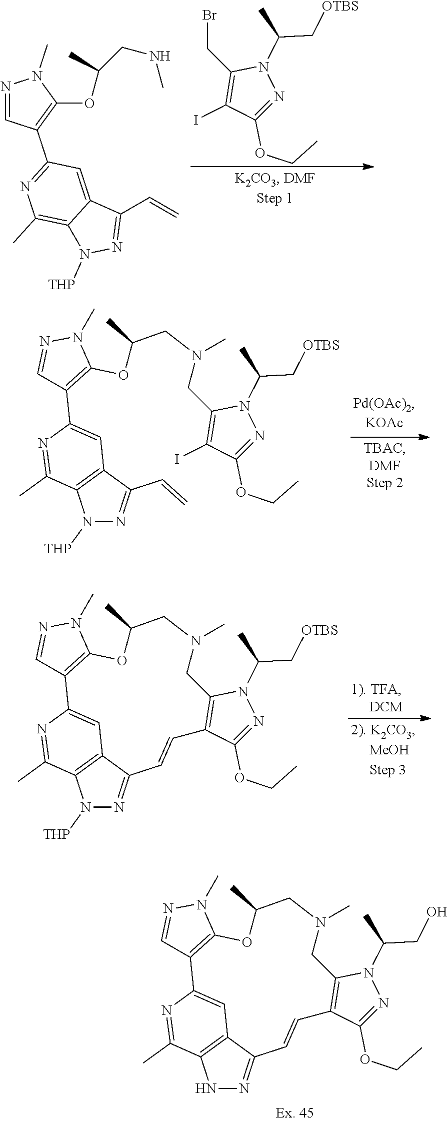

Step 1. To a solution of (2S)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine, which was prepared according to the method described in Ex. 29, (200 mg, 487 mol, 1 eq) in DMF (3 mL) was added [(2S)-2-[5-(bromomethyl)-3-ethoxy-4-iodo-pyrazol-1-yl]propoxy]-tert-butyl-dimethyl-silane, which was prepared according to the method described in Ex. 19, (245 mg, 487 mol, 1 eq) and K₂CO₃ (202 mg, 1.46 mmol, 3 eq). The mixture was stirred at 80° C. for 2 hr. On completion, the reaction mixture diluted with H₂O (30 mL) and extracted with EA (10 mL×3). The combined organic layers were washed with H₂O (50 mL), dried over Na₂SO₄, filtered and concentrated under reduced pressure to give a residue. The residue was purified by column chromatography (SiO₂, Petroleum ether/THF=1:0 to 1:1) to give (2S)-N-[[2-[(1S)-2-[tert-butyl(dimethyl)silyl]oxy-1-methyl-ethyl]-5-ethoxy-4-iodo-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (254 mg, 256 mol, 53% yield, 84% purity) as an orange oil. LCMS: (M+1: 833.2).

Step 2. To a solution of (2S)-N-[[2-[(1S)-2-[tert-butyl(dimethyl)silyl]oxy-1-methyl-ethyl]-5-ethoxy-4-iodo-pyrazol-3-yl]methyl]-N-methyl-2-[2-methyl-4-(7-methyl-1-tetrahydropyran-2-yl-3-vinyl-pyrazolo[3,4-c]pyridin-5-yl) pyrazol-3-yl]oxy-propan-1-amine (230 mg, 276 mol, 1 eq) in DMF (3 mL) was added KOAc (136 mg, 1.38 mmol, 5 eq) and Pd(OAc)₂ (9.30 mg, 41.4 μmol, 0.15 eq) and TBAC (153 mg, 552 mol, 2 eq). The mixture was stirred at 80° C. for 3 h under N₂ atmosphere. On completion, the reaction mixture diluted with H₂O (15 mL) and extracted with EA (10 mL×3). The combined organic layers were washed with H₂O (30 mL), dried over Na₂SO₄, filtered and concentrated under reduced pressure to give (10S,17E)-14-[(2S)-1-{[tert-butyl(dimethyl)silyl]oxy}propan-2-yl]-16-ethoxy-8,10,12,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (180 mg, crude) as a brown solid. LCMS: (M+1: 705.3).

Step 3. To a solution of (10S,17E)-14-[(2S)-1-{[tert-butyl(dimethyl)silyl]oxy}propan-2-yl]-16-ethoxy-8,10,12,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (90.0 mg, 128 μmol, 1 eq) in DCM (2 mL) was added TFA (1.35 mmol, 0.1 mL, 10.5 eq). The mixture was stirred at 25° C. for 1 hr. On completion, the mixture was concentrated in vacuum to give a black-brown oil (120 mg, crude).

This black-brown oil (60.0 mg, 100 mol, 1 eq) was dissolved in MeOH (1 mL), and K₂CO₃ (41.3 mg, 299 μmol, 3 eq) was added. The mixture was stirred at 25° C. for 1 hr. On completion, the reaction solution was filtered and the filtrate was purified by prep-HPLC purification (column: Phenomenex luna C₁₈ 150*25 mm*10 um; mobile phase: [water(FA)-ACN]; gradient: 13%-43% B over 8 min) to give Ex. 45 (23.42 mg, 41.65 μmol, 41.83% yield, 90.09% purity) as a yellow solid. LCMS: (M+1: 507.2). ¹H NMR for Ex. 45 is shown in the below NMR table.

### Preparation of (2S)-2-[(10S,17E)-6,8,10,12,16-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 46)

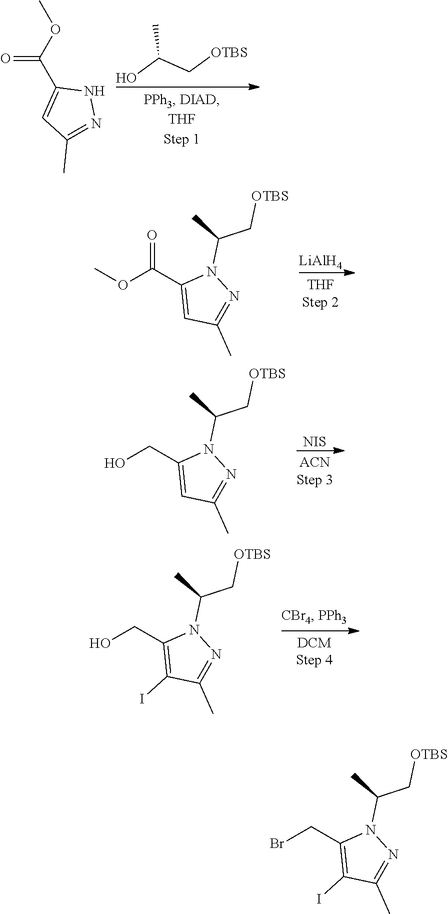

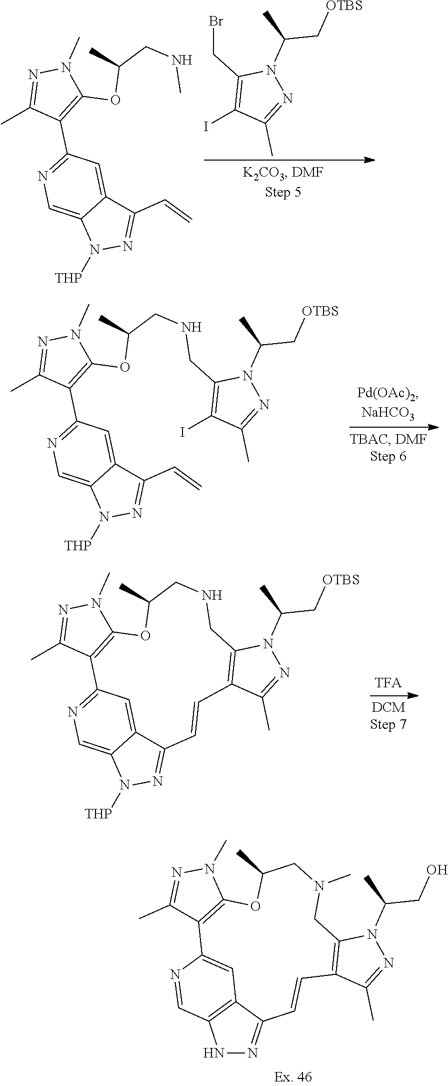

Step 1. A mixture of (2R)-1-[tert-butyl(dimethyl)silyl]oxypropan-2-ol, which was prepared according to the method described in Ex. 19, (7.00 g, 36.7 mmol, 1 eq), commercially available methyl 3-methyl-1H-pyrazole-5-carboxylate (10.3 g, 73.5 mmol, 2 eq), PPh₃ (14.4 g, 55.1 mmol, 1.5 eq), DIAD (55.1 mmol, 11 mL, 1.5 eq) in THE (50 mL) was degassed and purged with N₂ for 3 times, and then the mixture was stirred at 0° C. for 12 hours under N₂ atmosphere. On completion, the reaction mixture was concentrated under reduced pressure to remove solvent. The residue was purified by column chromatography (SiO₂, Petroleum ether/Ethyl acetate=10:1) to give the methyl 2-[(1S)-2-[tert-butyl(dimethyl)silyl]oxy-1-methyl-ethyl]-5-methyl-pyrazole-3-carboxylate (8.00 g, 25.6 mmol, 69% yield) as a white solid. LCMS: (M+1:313.1).

Step 2. To a solution of methyl 2-[(1S)-2-[tert-butyl(dimethyl)silyl]oxy-1-methyl-ethyl]-5-methyl-pyrazole-3-carboxylate (8.00 g, 25.6 mmol, 1 eq) in THE (15 mL) was added LiAlH₄ (2.5 M, 21 mL, 2 eq). The mixture was stirred at 0° C. for 2 h. On completion, the reaction mixture quenched with H₂O (1.5 mL), sodium hydroxide aqueous solution (1.5 mL), H₂O (4.5 mL). The residue was diluted with water (15 mL) and extracted with EA (15 mL×3). The combined organic layer was washed with the saturated solution of NaCl (30 mL), concentrated under reduced pressure to give a residue. The residue was purified by column chromatography (SiO₂, Petroleum ether/Ethyl acetate=3:1) to give 2-[(1S)-2-[tert-butyl(dimethyl)silyl]oxy-1-methyl-ethyl]-5-methyl-pyrazol-3-yl]methanol (4.00 g, 14.0 mmol, 54% yield) as a white solid. 1H NMR (400 MHz, CDCl₃) δ=6.00 (s, 1H), 4.68-4.59 (m, 2H), 3.94-3.81 (m, 2H), 3.38-3.31 (m, 1H), 2.30 (s, 3H), 1.55 (d, J=6.8 Hz, 3H), 0.86-0.82 (m, 9H), −0.02 (d, J=12.8 Hz, 6H). LCMS: (M+1:285.0).

Step 3. To a solution of [2-[(1S)-2-[tert-butyl(dimethyl)silyl]oxy-1-methyl-ethyl]-5-methyl-pyrazol-3-yl]methanol (1.80 g, 6.33 mmol, 1 eq) in ACN (20 mL) was added NIS (1.71 g, 7.59 mmol, 1.2 eq). The mixture was stirred at 0° C. for 2 hours. On completion, the reaction mixture was quenched by addition of H₂O (3 mL), and then diluted with H₂O (10 mL) and extracted with EA (10 mL×3). The combined organic layers were washed with the saturated solution of NaCl (10 mL), concentrated under reduced pressure to give [2-[(1S)-2-[tert-butyl(dimethyl)silyl]oxy-1-methyl-ethyl]-4-iodo-5-methyl-pyrazol-3-yl]methanol (2.50 g, 6.09 mmol, 96% yield) as a white solid. LCMS: (M+1:410.9).

Step 4. A mixture of [2-[(1S)-2-[tert-butyl(dimethyl)silyl]oxy-1-methyl-ethyl]-4-iodo-5-methyl-pyrazol-3-yl]methanol (2.30 g, 5.60 mmol, 1 eq), CBr₄ (2.23 g, 6.73 mmol, 1.2 eq), PPh₃ (1.76 g, 6.73 mmol, 1.2 eq) in DCM (15 mL) was degassed and purged with N₂ for 3 times, and then the mixture was stirred at 0° C. for 2 hours under N₂ atmosphere. On completion, the reaction mixture was concentrated under reduced pressure to remove solvent. The residue was purified by column chromatography (SiO₂, Petroleum ether/Ethyl acetate=10:1) to give [(2S)-2-[5-(bromomethyl)-4-iodo-3-methyl-pyrazol-1-yl]propoxy]-tert-butyl-dimethyl-silane (1.00 g, 2.11 mmol, 37% yield) as a white solid. LCMS: (M+1:474.9).

Step 5. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (200 mg, 487 μmol, 1 eq) and [(2S)-2-[5-(bromomethyl)-4-iodo-3-methyl-pyrazol-1-yl]propoxy]-tert-butyl-dimethyl-silane (345 mg, 730 mol, 1.5 eq) in DMF (5 mL) was added K₂CO₃ (202 mg, 1.46 mmol, 3 eq). The mixture was stirred at 80° C. for 12 hours. On completion, the reaction mixture was concentrated under reduced pressure to remove solvent. The residue was purified by column chromatography (SiO₂, Petroleum ether/THF=3:1 to 1:1) to give (2S)-N-[[2-[(1S)-2-[tert-butyl(dimethyl)silyl]oxy-1-methyl-ethyl]-4-iodo-5-methyl-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (200 mg, 249 μmol, 51% yield) as a yellow solid. LCMS: (M+1:803.1).

Step 6. A mixture of (2S)-N-[[2-[(1S)-2-[tert-butyl(dimethyl)silyl]oxy-1-methyl-ethyl]-4-iodo-5-methyl-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (170 mg, 211 μmol, 1 eq), Pd(OAc)₂ (9.51 mg, 42.3 mol, 0.2 eq), NaHCO₃ (44.4 mg, 529 μmol, 2.5 eq) and TBAC (211 mol, 60 μL, 1 eq) in DMF (10 mL) was degassed and purged with N₂ for 3 times, and then the mixture was stirred at 80° C. for 5 hours under N₂ atmosphere. On completion, the reaction mixture was quenched by addition of H₂O (20 mL) and extracted with EA (10 mL×3). The combined organic layers were washed with the saturated solution of NaCl (30 mL), concentrated under reduced pressure to give a residue. The residue was purified by column chromatography (SiO₂, Petroleum ether/THF=1:1) to give (10S,17E)-14-[(2S)-1-{[tert-butyl(dimethyl)silyl]oxy}propan-2-yl]-6,8,10,12,16-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (130 mg, 192 mol, 90% yield) as a white solid. LCMS: (M+1:675.3).

Step 7. To a solution of (10S,17E)-14-[(2S)-1-{[tert-butyl(dimethyl)silyl]oxy}propan-2-yl]-6,8,10,12,16-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (100 mg, 148 μmol, 1 eq) in DCM (3 mL) was added TFA (148 mol, 15.0 μL, 1 eq). The mixture was stirred at 25° C. for 0.5 hour. On completion, the reaction mixture was concentrated under reduced pressure to remove solvent. The crude product was purified by reversed-phase column chromatography: Phenomenex luna C18 150*25 mm*10 um; mobile phase: [water(FA)-ACN]; gradient:6%-36% B over 10 min to give Ex. 46 (13.76 mg, 27.14 μmol, 18.32% yield, 94% purity) as a white solid. LCMS: (M+1:477.1). ¹H NMR for Ex. 46 is shown in the below NMR table.

### Preparation of 2-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 47)

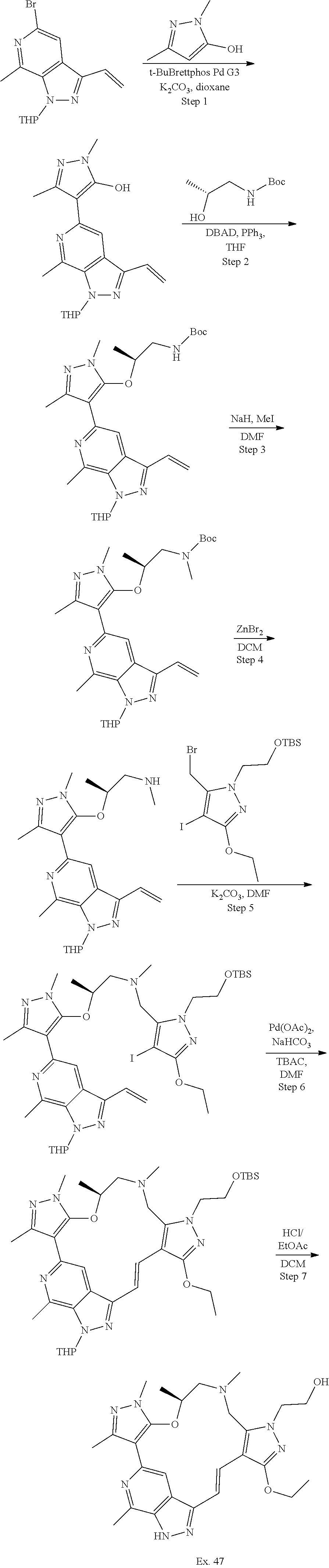

Step 1. To a mixture of 5-bromo-7-methyl-1-tetrahydropyran-2-yl-3-vinyl-pyrazolo[3,4-c]pyridine, which was prepared according to the methods described in Ex. 28, (1.10 g, 3.41 mmol, 1 eq), commercially available 2,5-dimethylpyrazol-3-ol (498 mg, 4.44 mmol, 1.3 eq), K₂CO₃ (1.42 g, 10.24 mmol, 3 eq) in dioxane (11 mL) was added [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (117 mg, 136 μmol, 0.04 eq) and purged with N₂ for 3 times, and then the mixture was stirred at 110° C. for 1.5 h under N₂ atmosphere. On completion, the reaction mixture was filtered and the filtrate concentrated under reduced pressure to give a residue. The residue was purified by CombiFlash (20 g silica gel column, THE in PE from 0-100%) to give 2,5-dimethyl-4-(7-methyl-1-tetrahydropyran-2-yl-3-vinyl-pyrazolo[3,4-c]pyridin-5-yl)pyrazol-3-ol (0.990 g, 2.80 mmol, 82% yield) as a yellow oil. LCMS: (M+1:354.1).

Step 2. To a solution of 2,5-dimethyl-4-(7-methyl-1-tetrahydropyran-2-yl-3-vinyl-pyrazolo[3,4-c]pyridin-5-yl)pyrazol-3-ol (970 mg, 2.74 mmol, 1 eq) in THE (10 mL) was added commercially available tert-butyl N-[(2R)-2-hydroxypropyl]carbamate (962 mg, 5.49 mmol, 2 eq) and PPh₃ (1.44 g, 5.49 mmol, 2 eq) and DBAD (948 mg, 4.12 mmol, 1.5 eq). The mixture was stirred at 25° C. for 2 h. On completion, the mixture was filtered and concentrated under reduced pressure to give a residue. The residue was purified by CombiFlash (20 g silica gel column, THE in PE from 0-100%) to give tert-butyl N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]carbamate (1.75 g, crude) as a yellow oil. ¹H NMR (400 MHz, DMSO-d₆) δ=6.79 (s, 1H), 6.59 (d, J=8.0 Hz, 1H), 6.16 (br d, J=8.0 Hz, 1H), 5.59 (d, J=3.6 Hz, 1H), 5.37 (s, 2H), 4.06 (s, 6H), 1.58 (q, J=7.6 Hz, 3H), 0.49-0.25 (m, 17H). LCMS: (M+1:511.6).

Step 3. To a solution of tert-butyl N-[(2S)-2-[2,5-dimethyl-4-(7-methyl-1-tetrahydropyran-2-yl-3-vinyl-pyrazolo [3,4-c]pyridin-5-yl)pyrazol-3-yl]oxypropyl]carbamate (1.70 g, 3.33 mmol, 1 eq) in DMF (17 mL) was added NaH (399 mg, 9.99 mmol, 60% purity, 3 eq) and CH₃I (709 mg, 4.99 mmol, 311 μL, 1.5 eq). The mixture was stirred at 20° C. for 1 h. On completion, the reaction mixture was quenched by addition H₂O (70 mL) at 25° C., and extracted with EA (50 mL×3). The combined organic layers were washed with H₂O (40 mL×2), dried over Na₂SO₄, filtered and concentrated under reduced pressure to give a residue the residue was purified by CombiFlash (20 g silica gel column, THE in PE from 0-100%) to give tert-butyl N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]-N-methyl-carbamate (1.76 g, 1.68 mmol, 50% yield, 50% purity) as a yellow solid. LCMS: (M+1:525.3).

Step 4. To a solution of tert-butyl N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]-N-methyl-carbamate (1.70 g, 3.24 mmol, 1 eq) in DCM (17 mL) was added ZnBr₂ (3.65 g, 16.2 mmol, 811 μL, 5 eq). The mixture was stirred at 25° C. for 2 h. On completion, the reaction mixture was quenched by addition H₂O (25 mL) at 25° C., and extracted with DCM (15 mL×4), dried over Na₂SO₄, filtered and concentrated under reduced pressure to give a residue. The residue was purified by CombiFlash (20 g silica gel column, THF in PE from 0-100%) to give (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (550 mg, 1.30 mmol, 40% yield) as a white solid. ¹H NMR (400 MHz, MeOD-d₄) δ=7.82 (d, J=3.2 Hz, 1H), 7.05 (dd, J=11.6, 18.0 Hz, 1H), 6.16 (d, J=18.0 Hz, 1H), 6.07-6.01 (m, 1H), 5.59 (d, J=11.6 Hz, 1H), 4.42-4.32 (m, 1H), 4.04-3.95 (m, 1H), 3.90-3.80 (m, 1H), 3.71 (s, 3H), 3.17-3.10 (m, 1H), 3.03-2.98 (m, 4H), 2.70-2.61 (m, 1H), 2.58 (d, J=2.4 Hz, 3H), 2.25 (d, J=1.2 Hz, 3H), 2.22-2.15 (m, 2H), 1.92-1.78 (m, 1H), 1.75-1.61 (m, 2H), 1.14 (dd, J=2.8, 6.4 Hz, 3H). LCMS: (M+1:425.2).

Step 5. To a solution of (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (150 mg, 353 μmol, 1 eq) and 2-15-(bromomethyl)-3-ethoxy-4-iodo-pyrazol-1-yl]ethoxy-tert-butyl-dimethyl-silane, prepared according to the method described in Ex. 1, (207 mg, 424 μmol, 1.2 eq) in DMF (1.5 mL) was added K₂CO₃ (146 mg, 1.06 mmol, 3 eq). The mixture was stirred at 80° C. for 1 h. On completion, the reaction mixture was quenched by addition H₂O (8 mL) at 25° C., and extracted with EA (8 mL×3). The combined organic layers were washed with H₂O (10 mL×2), dried over Na₂SO₄, filtered and concentrated under reduced pressure to give a residue. The residue was purified by CombiFlash (4 g silica gel column, THE in PE from 0-100%) to give (2S)-N-[[2-[2-[tert-butyl(dimethyl)silyl]oxyethyl]-5-ethoxy-4-iodo-pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (200 mg, 240 μmol, 68% yield) as a yellow solid. LCMS: (M+1:833.2).

Step 6. A mixture of (2S)-N-[[2-[2-[tert-butyl(dimethyl)silyl]oxyethyl]-5-ethoxy-4-iodo-pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (190 mg, 228 mol, 1 eq), TBAC (63.4 mg, 228 μmol, 1 eq), NaHCO₃ (47.9 mg, 570 μmol, 2.5 eq) and Pd(OAc)₂ (10.2 mg, 45.6 μmol, 0.2 eq) in DMF (2 mL) was degassed and purged with N₂ for 3 times, and then the mixture was stirred at 130° C. for 1.5 h under N₂ atmosphere. On completion, the reaction mixture was quenched by addition of H₂O (8 mL) at 25° C. and extracted with EA (8 mL×3). The combined organic layers were washed with H₂O (10 mL×2), dried over Na₂SO₄, filtered and concentrated under reduced pressure to give (10S,17E)-14-(2-{[tert-butyl(dimethyl)silyl]oxy}ethyl)-16-ethoxy-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (150 mg, 212 μmol, 93% yield) as a brown oil. LCMS: (M+1:705.3).

Step 7. To a solution of (10S,17E)-14-(2-{[tert-butyl(dimethyl)silyl]oxy}ethyl)-16-ethoxy-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (140 mg, 198 μmol, 1 eq) in DCM (3 mL) was added HCl/EtOAc (4 M, 49.6 μL, 1 eq). The mixture was stirred at 25° C. for 1 hr. On completion, the mixture was filtered and concentrated under reduced pressure to give a residue. The residue was purified by prep-HPLC (column: Phenomenex luna C18 150*25 mm*10 um; mobile phase: [water(FA)-ACN]; gradient:10%-40% B over 10 min) to give Ex. 47 (25.48 mg, 50.30 mol, 25.33% yield) as a yellow solid. LCMS: (M+1:507.1). ¹H NMR for Ex. 47 is shown in the below NMR table.

### Preparation of (10S,17E)-14-[(2S)-1-hydroxypropan-2-yl]-6,8,10,12-tetramethyl-2,10,11,12,13,14-hexahydro-8H-5,3-(azenometheno)tripyrazolo[3,4-f:3′,4′-j:4″,3″-n∥1,4]oxazacyclopentadecine-16-carbonitrile (Ex. 48)

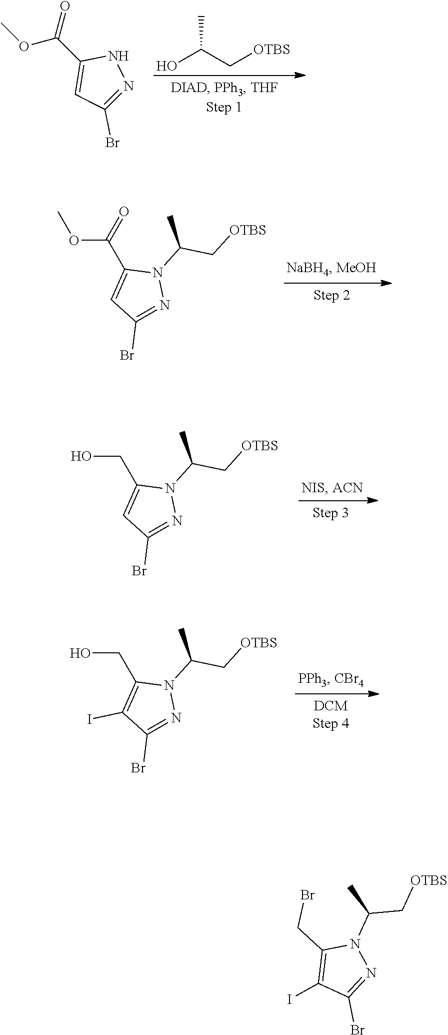

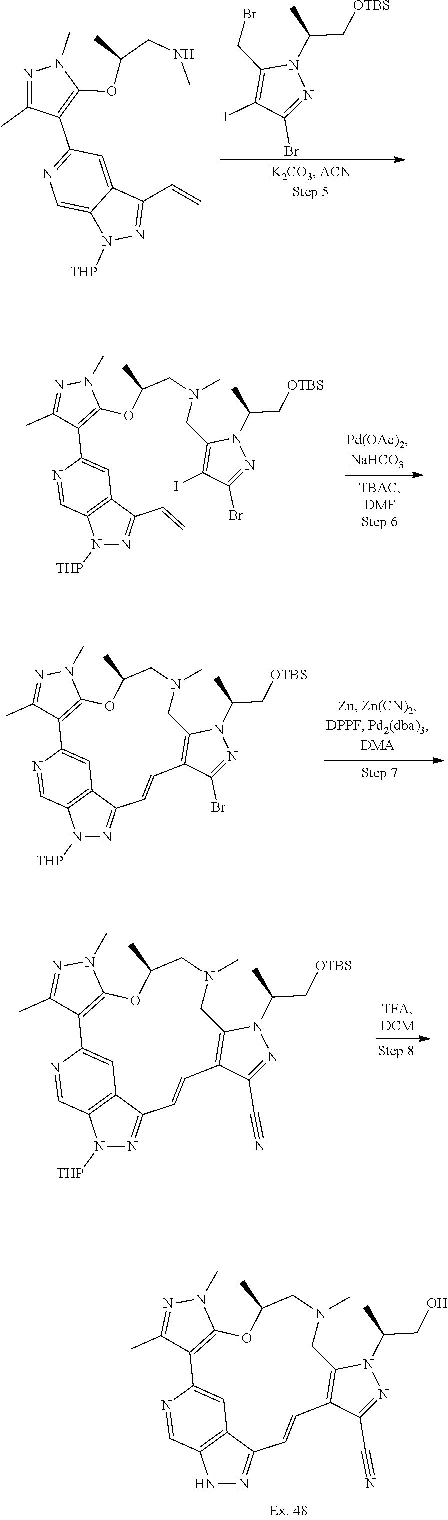

Step 1. A solution of commercially available methyl 3-bromo-1H-pyrazole-5-carboxylate (2.00 g, 9.76 mmol, 1 eq), (2R)-1-[tert-butyl(dimethyl)silyl]oxypropan-2-ol, which was prepared according to the method described in Ex. 19, (1.86 g, 9.76 mmol, 1 eq) and PPh₃ (5.12 g, 19.51 mmol, 2 eq) in THE (40 mL) was stirred at 25° C. for 30 mines. Then DIAD (3.95 g, 19.51 mmol, 2 eq) was added at 0° C., the mixture and stirred at 25° C. for 2 h. On completion, the mixture was concentrated to give a residue. The residue was purified by column chromatography (SiO₂, PE:THF=1:0 to 8:1) to give methyl 5-bromo-2-[(1S)-2-[tert-butyl(dimethyl)silyl]oxy-1-methyl-ethyl]pyrazole-3-carboxylate (3.21 g, 8.51 mmol, 87% yield) as a colorless oil. LCMS: (M+1:376.9).

Step 2. To a mixture of methyl 5-bromo-2-[(1S)-2-[tert-butyl(dimethyl)silyl]oxy-1-methyl-ethyl]pyrazole-3-carboxylate (1.10 g, 2.92 mmol, 1 eq) in MeOH (11 mL) was added NaBH₄ (1.10 g, 29.1 mmol, 10 eq) at 0° C., the reaction mixture was stirred at 25° C. for 8 h. On completion, the mixture was quenched with water (20 mL) and extracted with ethyl acetate (15 mL×3). The combined organic phase was dried over anhydrous sodium sulfate, filtered and concentrated to give a residue. The residue was purified by column chromatography (SiO₂, PE:THF=1:0 to 10:1) to give [5-bromo-2-[(1S)-2-[tert-butyl(dimethyl)silyl]oxy-1-methyl-ethyl]pyrazol-3-yl]methanol (779 mg, 2.23 mmol, 76% yield) as a colorless oil. LCMS: (M+1:348.9).

Step 3. To a solution of [5-bromo-2-[(1S)-2-[tert-butyl(dimethyl)silyl]oxy-1-methyl-ethyl]pyrazol-3-yl]methanol (729 mg, 2.09 mmol, 1 eq) in ACN (7.5 mL) was added NIS (2.35 g, 10.4 mmol, 5 eq). The mixture was stirred at 40° C. for 1 h. On completion, the mixture was quenched with sat. Na₂SO₃ (20 mL) and extracted with ethyl acetate (15 mL×3). The combined organic phase was dried over anhydrous sodium sulfate, filtered and concentrated to give a residue. The residue was purified by column chromatography (SiO₂, PE:THF=1:0 to 6:1) to give [5-bromo-2-[(1S)-2-[tert-butyl(dimethyl)silyl]oxy-1-methyl-ethyl]-4-iodo-pyrazol-3-yl]methanol (720 mg, 1.52 mmol, 72% yield) as a colorless oil. LCMS: (M+1:474.9).

Step 4. A solution of [5-bromo-2-[(1S)-2-[tert-butyl(dimethyl)silyl]oxy-1-methyl-ethyl]-4-iodo-pyrazol-3-yl]methanol (295 mg, 620 μmol, 1 eq) and PPh₃ (195 mg, 744 mol, 1.2 eq) in DCM (3 mL) was degassed and purged with N₂ for 3 times, and then CBr₄ (247 mg, 744 mol, 1.2 eq) was added at 0° C. The mixture was stirred at 25° C. for 1 h under N₂ atmosphere. On completion, the mixture was concentrated to give a residue. The residue was purified by column chromatography (SiO₂, Petroleum ether/Ethyl acetate=1:0 to 9:1) to give [(2S)-2-[3-bromo-5-(bromomethyl)-4-iodo-pyrazol-1-yl]propoxy]-tert-butyl-dimethyl-silane (271 mg, 503 μmol, 81% yield) as a colorless oil. LCMS: (M+1:538.6).

Step 5. To a solution of (2S)-2-[2,5-dimethyl-4-(1-tetrahydropyran-2-yl-3-vinyl-pyrazolo|3,4-c|pyridin-5-yl)pyrazol-3-yl]oxy-N-methyl-propan-1-amine, which was prepared according to the method described in Ex. 22, (206 mg, 501 mol, 1 eq) in ACN (5 mL) was added [(2S)-2-[3-bromo-5-(bromomethyl)-4-iodo-pyrazol-1-yl]propoxy]-tert-butyl-dimethyl-silane (270 mg, 501 mol, 1 eq) and K₂CO₃ (208 mg, 1.51 mmol, 3 eq). The mixture was stirred at 60° C. for 1 h. On completion, the mixture was filtered and concentrated to give a residue. The residue was purified by column chromatography (SiO₂, PE:THF=1:0 to 2:1) to give (2S)-N-[[5-bromo-2-[(1S)-2-[tert-butyl(dimethyl)silyl]oxy-1-methyl-ethyl]-4-iodo-pyrazol-3-yl]methyl]-2-[2,5-dimeth yl-4-(1-tetrahydropyran-2-yl-3-vinyl-pyrazolo[3,4-c]pyridin-5-yl)pyrazol-3-yl]oxy-N-methyl-propan-1-amine (320 mg, 368 μmol, 73% yield) as a yellow oil. LCMS: (M+1:869.1).

Step 6. A mixture of (2S)-N-[[5-bromo-2-[(1S)-2-[tert-butyl(dimethyl)silyl]oxy-1-methyl-ethyl]-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (300 mg, 345 μmol, 1 eq), NaHCO₃ (72.6 mg, 864 mol, 2.5 eq), TBAC (96.0 mg, 345 μmol, 1 eq) and Pd(OAc)₂ (15.5 mg, 69.1 μmol, 0.2 eq) in DMF (15 mL) was degassed and purged with N₂ for 3 times, and then the mixture was stirred at 130° C. for 1 h under N₂ atmosphere. On completion, the mixture was quenched with water (20 mL) and extracted with ethyl acetate (15 mL×3). The combined organic phase was dried over anhydrous sodium sulfate, filtered and concentrated to give a residue. The residue was purified by column chromatography (SiO₂, PE:THF=1:0 to 1:1) to give (10S,17E)-16-bromo-14-[(2S)-1-{[tert-butyl(dimethyl)silyl]oxy}propan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (180 mg, 243 mol, 70% yield) as a purple solid. LCMS: (M+1:741.2).

Step 7. To a solution of (10S,17E)-16-bromo-14-[(2S)-1-{[tert-butyl(dimethyl)silyl]oxy}propan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (160 mg, 216 μmol, 1 eq) in DMA (1.6 mL) was added Zn (3.54 mg, 54.0 μmol, 0.25 eq), dicyanozinc (27.9 mg, 237 μmol, 1.1 eq), Pd₂(dba)₃ (6.54 mg, 7.14 μmol, 0.033 eq) and DPPF (10.1 mg, 18.3 mol, 0.085 eq) under N₂. The mixture was stirred at 120° C. for 16 h. On completion, the mixture was quenched with water (5 mL) and extracted with EA (5 mL×3). The combined organic layers were washed with brine (10 mL), dried over Na₂SO₄, filtered and concentrated under reduced pressure to give (10S,17E)-14-[(2S)-1-{[tert-butyl(dimethyl)silyl]oxy}propan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n∥1,4]oxazacyclopentadecine-16-carbonitrile (180 mg, crude) as a purple solid. LCMS: (M+1:686.3).

Step 8. To a solution of (10S,17E)-14-[(2S)-1-{[tert-butyl(dimethyl)silyl]oxy}propan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine-16-carbonitrile (160 mg, 233 mol, 1 eq) in DCM (4 mL) was added TFA (10.7 mmol, 800 μL, 46.2 eq). The mixture was stirred at 25° C. for 1 h. On completion, the mixture was concentrated under reduced pressure to give a residue. The residue was purified by prep-HPLC (column: Waters xbridge 150*25 mm 10 um; mobile phase: [water(NH4HCO3)-ACN]; gradient:34%-64% B over 10 min) to give Ex. 48 (24.53 mg, 49.92 μmol, 21.40% yield, 99.219% purity) as an off-white solid. LCMS: (M+1:488.2). ¹H NMR for Ex. 48 is shown in the below NMR table.

### Preparation of {[(11R,17E)-14-(2-hydroxyethyl)-6,8,11,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 49)

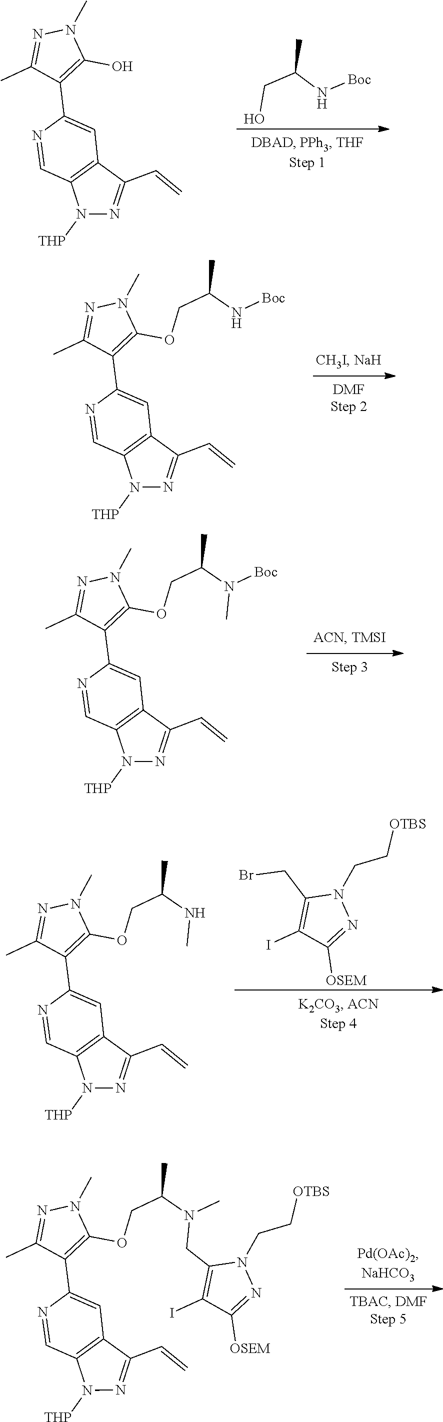

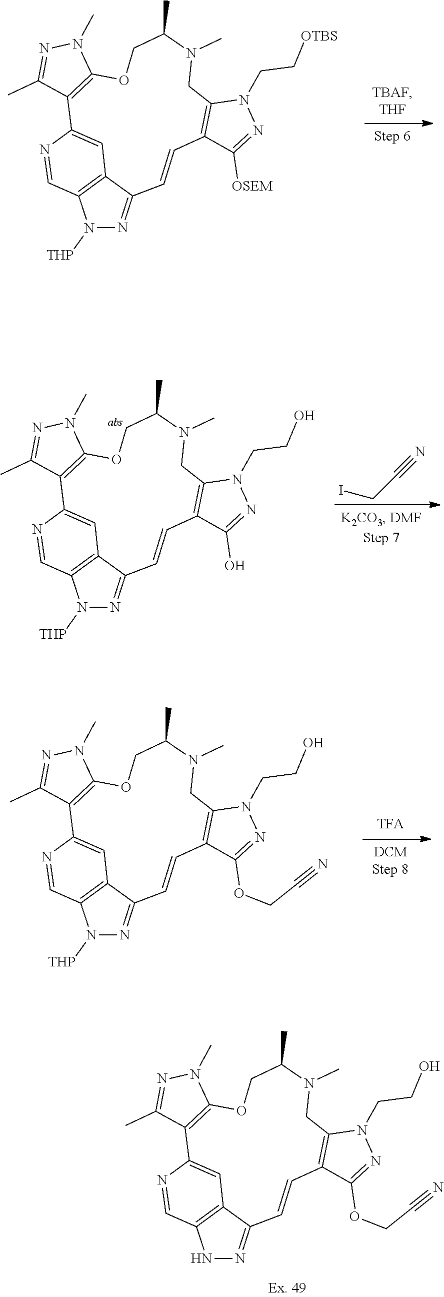

Step 1. To a solution of commercially available tert-butyl N-[(1R)-2-hydroxy-1-methyl-ethyl]carbamate (309 mg, 1.77 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (600 mg, 1.77 mmol, 1 eq) in THF (14 mL) was added PPh₃ (927 mg, 3.54 mmol, 2 eq) and DBAD (611 mg, 2.65 mmol, 1.5 eq). The mixture was stirred at 0° C. for 2 h under N₂. On completion, the mixture was concentrated to give a residue. The residue was purified by column chromatography (SiO₂, PE/THF=1:0 to 1:1) to give tert-butyl N-[(1R)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]carbamate (1.20 g, 942 mol, 53% yield, 39% purity) as a white solid. LCMS: (M+1:497.1).

Step 2. To a solution of tert-butyl N-[(1R)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]carbamate (1.00 g, 2.01 mmol, 1 eq) in DMF (10 mL) was added NaH (161 mg, 4.03 mmol, 60% purity, 2 eq) and MeI (428 mg, 3.02 mmol, 1.5 eq). The mixture was stirred at 0° C. for 2 h. On completion, the mixture was quenched with water (20 mL) and extracted with EA (25 mL×3). The combined organic phase was dried over anhydrous sodium sulfate, filtered and concentrated to give a residue. The residue was purified by column chromatography (SiO₂, PE/THF=1:1 to 1:1) to give tert-butyl N-[(1R)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]-N-methyl-carbamate (1.10 g, 861 μmol, 42% yield, 40% purity) as a white solid. LCMS: (M+1: 511.3).

Step 3. To a solution of tert-butyl N-[(1R)-2-[2,5-dimethyl-4-(1-tetrahydropyran-2-yl-3-vinyl-pyrazolo[3,4-c]pyridin-5-yl)pyrazol-3-yl]oxy-1-methyl-ethyl]-N-methyl-carbamate (1.10 g, 2.15 mmol, 1 eq) in ACN (10 mL) was added TMSI (560 mg, 2.80 mmol, 1.3 eq). The mixture was stirred at 25° C. for 2 h. On completion, the mixture was quenched with water (20 mL) and extracted with EA (25 mL×3). The combined organic phase was dried over anhydrous sodium sulfate, filtered and concentrated to give (2R)-1-[2,5-dimethyl-4-(1-tetrahydropyran-2-yl-3-vinyl-pyrazolo[3,4-c]pyridin-5-yl)pyrazol-3-yl]oxy-N-methyl-propan-2-amine (800 mg, 1.95 mmol, 90% yield) as a white solid. LCMS: (M+1:411.1).

Step 4. To a solution of (2R)-1-[2,5-dimethyl-4-(1-tetrahydropyran-2-yl-3-vinyl-pyrazolo[3,4-c]pyridin-5-yl)pyrazol-3-yl]oxy-N-methyl-propan-2-amine (500 mg, 609 mol, 1 eq) and 2-[[5-(bromomethyl)-1-[2-[tert-butyl(dimethyl)silyl]oxyethyl]-4-iodo-pyrazol-3-yl]oxymethoxy|ethyl-trimethyl-silane, which was prepared according to the method described in Ex. 42, (360 mg, 609 μmol, 1 eq) in ACN (10 mL) was added K₂CO₃ (252 mg, 1.83 mmol, 3 eq). The mixture was stirred at 60° C. for 2 h. On completion, the mixture was filtered and concentrated to give a residue. The residue was purified by column chromatography (SiO₂, PE/THF=1:1 to 1:1) to give (2R)-N-[[2-[2-[tert-butyl(dimethyl)silyl]oxyethyl]-4-iodo-5-(2-trimethylsilylethoxymethoxy) pyrazol-3-yl]methyl]-1-[2,5-dimethyl-4-(1-tetrahydropyran-2-yl-3-vinyl-pyrazolo[3, 4-c]pyridin-5-yl)pyrazol-3-yl]oxy-N-methyl-propan-2-amine (1.10 g, 597 mol, 98% yield, 50% purity) as a white solid. LCMS: (M+1:921.4).

Step 5. To a solution of (2R)-N-[[2-[2-[tert-butyl(dimethyl)silyl]oxyethyl]-4-iodo-5-(2-trimethylsilylethoxymethoxy)pyrazol-3-yl]methyl]-1-[2,5-dimethyl-4-(1-tetrahydropyran-2-yl-3-vinyl-pyrazolo[3,4-c]pyridin-5-yl)pyrazol-3-yl]oxy-N-methyl-propan-2-amine (1.10 g, 1.19 mmol, 1 eq) in DMF (55 mL) was added TBAC (331 mg, 1.19 mmol, 1 eq), NaHCO₃ (250 mg, 2.99 mmol, 2.5 eq) and Pd(OAc)₂ (53.6 mg, 238 μmol, 0.2 eq). The mixture was stirred at 130° C. for 2 h under N₂. On completion, the mixture was quenched with water (80 mL) and extracted with ethyl acetate (80 mL×3). The combined organic phase was dried over anhydrous sodium sulfate, filtered and concentrated to give a residue. The residue was purified by column chromatography (SiO₂, DCM/MeOH=15:1) to give (11R,17E)-14-(2-{[tert-butyl(dimethyl)silyl]oxy}ethyl)-6,8,11,12-tetramethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (810 mg, 857 μmol, 72% yield, 84% purity) as a white solid. LCMS: (M+1:793.4).

Step 6. To a solution of (11R,17E)-14-(2-{[tert-butyl(dimethyl)silyl]oxy}ethyl)-6,8,11,12-tetramethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (400 mg, 504 μmol, 1 eq) in THE (10 mL) was added TBAF (1 M, 3 mL, 6 eq). The mixture was stirred at 70° C. for 12 h. On completion, the mixture was quenched with water (20 mL) and extracted with 2-MeTHF (25 mL×3). The combined organic phase was dried over anhydrous sodium sulfate, filtered and concentrated to give (11R,17E)-14-(2-hydroxyethyl)-6,8,11,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (270 mg, 492 μmol, 97% yield) as a white solid. LCMS: (M+1:549.2).

Step 7. To a solution of (11R,17E)-14-(2-hydroxyethyl)-6,8,11,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (200 mg, 364 mol, 1 eq) and commercially available 2-iodoacetonitrile (73.0 mg, 437 μmol, 1.2 eq) in DMF (4 mL) was added K₂CO₃(101 mg, 729 mol, 2 eq). The mixture was stirred at 80° C. for 1 h. On completion, the mixture was quenched with water (10 mL) and extracted with EA (10 mL×3). The combined organic phase was dried over anhydrous sodium sulfate, filtered and concentrated to give a residue. The residue was purified by column chromatography (SiO₂, DCM/MeOH=20:1 to 10:1) to give {[(11R,17E)-14-(2-hydroxyethyl)-6,8,11,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (150 mg, 255 μmol, 70% yield) as a yellow solid. LCMS: (M+1:588.2).

Step 8. To a solution of 1[(11R,17E)-14-(2-hydroxyethyl)-6,8,11,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (100 mg, 170 μmol, 1 eq) in DCM (5 mL) was added TFA (1.54 g, 13.5 mmol, 79.0 eq). The mixture was stirred at 25° C. for 2 h. On completion, the mixture was concentrated to give a residue. The crude product was purified by reversed-phase column chromatography (column: Waters xbridge150*25 mm 10 um; mobile phase: [water(NH₄HCO₃)-ACN]; gradient:19%-49% B over 10 min) to give Ex. 49 (16.73 mg, 33.22 mol, 11.15% yield) as a white solid. LCMS: (M+1:504.1). ¹H NMR for Ex. 49 is shown in the below NMR table.

### Preparation of 2-[(10S,17E)-8,10,12,16,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 51)

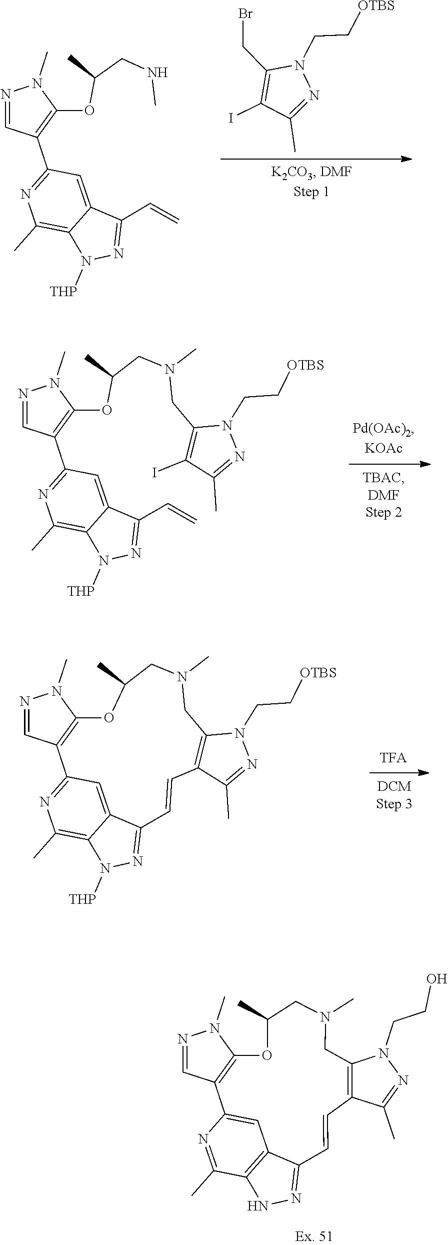

Step 1. To a solution of (2S)-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine, which was prepared according to the method described in Ex. 29, (200 mg, 487 mol, 1 eq) in DMF (3 mL) was added 2-[5-(bromomethyl)-4-iodo-3-methyl-pyrazol-1-yl]ethoxy-tert-butyl-dimethyl-silane, which was prepared according to the method described in Ex. 13 and 14, (224 mg, 487 μmol, 1 eq) and K₂CO₃ (202 mg, 1.46 mmol, 3 eq). The mixture was stirred at 80° C. for 2 h. On completion, the reaction mixture diluted with H₂O (9 mL) and extracted with EA (5 mL×3). The combined organic layers were washed with H₂O (10 mL), dried over Na₂SO₄, filtered and concentrated under reduced pressure to give a residue. The residue was purified by column chromatography (SiO₂, Petroleum ether/THF=1:0 to 1:1) to give (2S)-N-[[2-[2-[tert-butyl(dimethyl)silyl]oxyethyl]-4-iodo-5-methyl-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (290 mg, 342 mol, 70% yield, 93% purity) as an orange oil. LCMS: (M+1: 789.2).

Step 2. To a solution of (2S)-N-[[2-[2-[tert-butyl(dimethyl)silyl]oxyethyl]-4-iodo-5-methyl-pyrazol-3-yl]methyl]-N-methyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (140 mg, 177 mol, 1 eq) in DMF (1.5 mL) was added KOAc (87.1 mg, 887 μmol, 5 eq) and Pd(OAc)₂ (5.98 mg, 26.6 μmol, 0.15 eq) and TBAC (98.7 mg, 357 mol, 2 eq). The mixture was stirred at 80° C. for 2 h under N₂ atmosphere. On completion, the reaction mixture diluted with H2O (30 mL) and extracted with EA (15 mL×3). The combined organic layers were washed with H₂O (50 mL), dried over Na₂SO₄, filtered and concentrated under reduced pressure to give (10S,17E)-14-(2-{[tert-butyl(dimethyl)silyl]oxy}ethyl)-8,10,12,16,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (220 mg, crude) as a black brown solid. LCMS: (M+1: 661.5).

Step 3. To a solution of (10S,17E)-14-(2-{[tert-butyl(dimethyl)silyl]oxy}ethyl)-8,10,12,16,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (190 mg, 287 μmol, 1 eq) in DCM (4 mL) was added TFA (10.8 mmol, 0.8 mL, 37.5 eq). The mixture was stirred at 25° C. for 1 h. On completion, the reaction was concentrated in vacuum to give a residue. The residue was purified by prep-HPLC purification (column: Phenomenex luna Cis 150*25 mm*10 um; mobile phase: [water(FA)-ACN]; gradient: 3%-33% B over 10 min) to give Ex. 51 (8.63 mg, 15.67 μmol, 5.45% yield, 83.98% purity) as an off-white solid. LCMS: (M+1: 463.1). ¹H NMR for Ex. 51 is shown in the below NMR table.

### Preparation of {[(10S,17E)-14-[(2S)-1-hydroxypropan-2-yl]-6,8,10,12,20-pentamethyl-2,10,11,12,13,14-hexahydro-8H-5,3-(azenometheno)tripyrazolol3,4-f:3′,4′-j:4″,3″-n][1,4]oxazacyclopentadecin-16-yl]oxy}acetonitrile (Ex. 52)

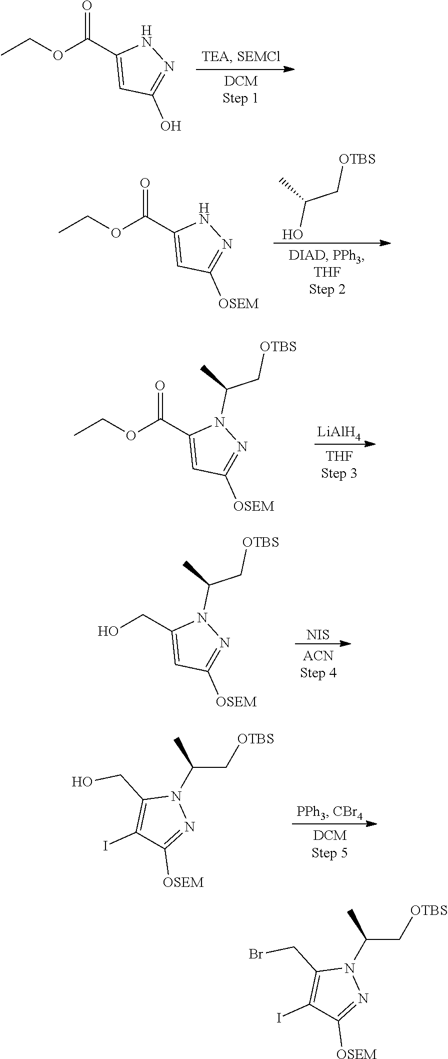

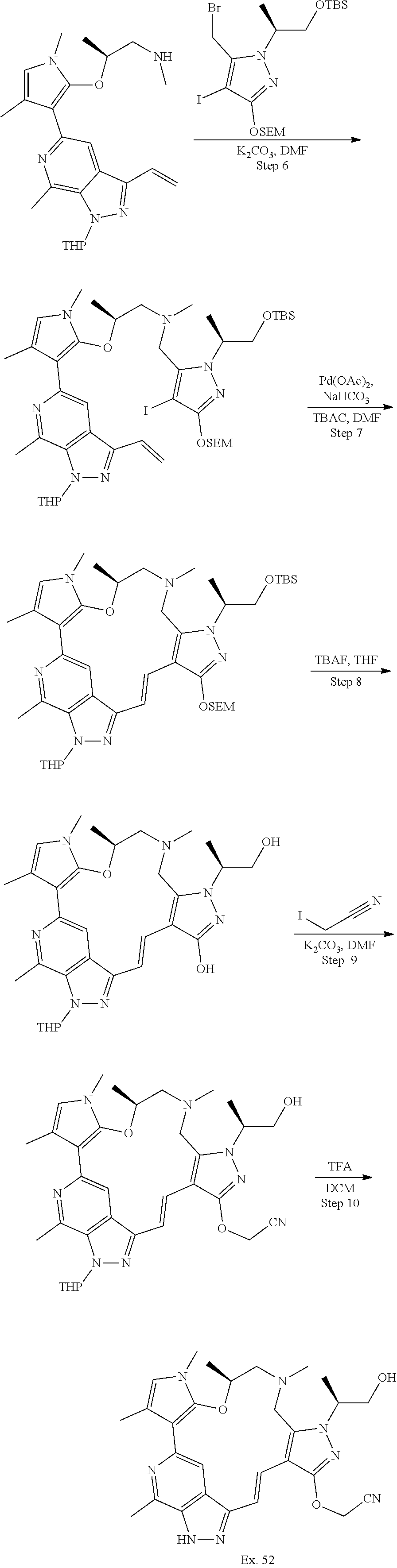

Step 1. Commercially available 5-hydroxy-1H-pyrazole-3-carboxylic acid ethyl ester (10.0 g, 64 mmol, 1 eq), SEM-Cl (42.7 g, 256 mmol, 4 eq), and TEA (38.9 g, 384 mmol, 6 eq) were mixed in DCM (100 mL) at 0° C. and the mixture was stirred at 25° C. for 1 h. On completion, the mixture was diluted with water (500 mL) and extracted with DCM (100 mL×3). The combined organic phase was dried over Na₂SO₄, filtered and the filtrate was concentrated to give a residue. The residue was purified by column chromatography on silica gel (120 g silica gel, EA in Petroleum ether from 0% to 100%) to give ethyl 3-(2-trimethylsilylethoxymethoxy)-1H-pyrazole-5-carboxylate (10.6 g, 35.8 mmol, 56% yield) as a colorless oil. LCMS: (M+1-28: 259.0).

Step 2. To the mixture of ethyl 3-(2-trimethylsilylethoxymethoxy)-1H-pyrazole-5-carboxylate (10.0 g, 34.9 mmol, 1 eq), (2R)-1-[tert-butyl(dimethyl)silyl]oxypropan-2-ol, which was prepared according to the method described in Ex. 19, (13.3 g, 69.8 mmol, 2 eq) and PPh₃ (20.1 g, 76.8 mmol, 2.2 eq) in THE (230 mL), was added DIAD (17.7 g, 87.3 mmol, 2.5 eq) at 0° C., and the resulting mixture was stirred for another 2 h at 25° C. under N₂. On completion, the reaction mixture was concentrated in vacuo. The residue was purified by column chromatography on silica gel (80 g silica gel, EA in Petroleum ether from 0% to 100%) to give ethyl 2-[(1S)-2-[tert-butyl(dimethyl)silyl]oxy-1-methyl-ethyl]-5-(2-trimethylsilylethoxymethoxy)pyrazole-3-carboxylate (16.1 g, 31.6 mmol, 90% yield) as a yellow oil. ¹H NMR (400 MHz, DMSO-d₆) δ=6.33-6.28 (m, 1H), 5.38-5.28 (m, 1H), 5.21-5.16 (m, 2H), 4.29-4.21 (m, 2H), 3.72-3.65 (m, 4H), 0.92-0.83 (m, 6H), 0.73 (s, 9H), 0.05-0.02 (m, 2H), −0.01-0.04 (m, 9H), −0.09 (s, 3H), −0.17 (s, 3H).

Step 3. To a solution of ethyl 2-[(1S)-2-[tert-butyl(dimethyl)silyl]oxy-1-methyl-ethyl]-5-(2-trimethylsilylethoxymethoxy)pyrazole-3-carboxylate (15.5 g, 33.8 mmol, 1 eq) in THE (155 mL) was added LiAlH₄ (2.50 M, 13.5 mL, 1 eq) at 0° C. The mixture was stirred at 25° C. for 0.5 h. On completion, the mixture was quenched by slow addition of water (1.5 mL), 15% sodium hydroxide solution (1.5 ml) and water (5 ml) at 0° C. The reaction mixture was filtered and concentrated under reduced pressure and the residue was purified by column chromatography on silica gel (40 g silica gel, EA in Petroleum ether from 0% to 100%) to give [2-[(1S)-2-[tert-butyl(dimethyl)silyl]oxy-1-methyl-ethyl]-5-(2-trimethylsilylethoxymethoxy)pyrazol-3-yl]methanol (7.25 g, 17.3 mmol, 51% yield) as a colorless oil. LCMS: (M+1: 417.1)

Step 4. To a solution of [2-[(1S)-2-[tert-butyl(dimethyl)silyl]oxy-1-methyl-ethyl]-5-(2-trimethylsilylethoxymethoxy)pyrazol-3-yl]methanol (6.70 g, 16.1 mmol, 1 eq) in ACN (70 mL) was added NIS (3.98 g, 17.7 mmol, 1.1 eq) at 0° C. for 0.5 h. The mixture was then stirred at 25° C. for 1.5 h. On completion, the reaction mixture was quenched by addition of saturated sodium sulfite solution (70 mL) at 0° C., and then diluted with H₂O (260 mL) and extracted with EA (100 mL×3). The combined organic layers were dried over Na₂SO₄, filtered, and concentrated under reduced pressure. The residue was purified by column chromatography on silica gel (20 g silica gel, EA in Petroleum ether from 0% to 100%) to give [2-[(1S)-2-[tert-butyl(dimethyl)silyl]oxy-1-methyl-ethyl]-4-iodo-5-(2-trimethylsilylethoxymethoxy)pyrazol-3-yl]methanol (7.60 g, 13.6 mmol, 85% yield) as a colorless oil. LCMS: (M+1: 543.1)

Step 5. To a solution of [2-[(1S)-2-[tert-butyl(dimethyl)silyl]oxy-1-methyl-ethyl]-4-iodo-5-(2-trimethylsilylethoxymethoxy)pyrazol-3-yl]methanol (7.00 g, 12.9 mmol, 1 eq), CBr₄ (5.13 g, 15.5 mmol, 1.2 eq) in DCM (70 mL) was added PPh₃ (4.06 g, 15.5 mmol, 1.2 eq) at 0° C. The mixture was stirred at 25° C. for 1.5 h. On completion, the reaction mixture was concentrated in vacuo. The residue was purified by column chromatography on silica gel (40 g silica gel, EA in Petroleum ether from 0% to 100%) to give 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane (3.50 g crude of ~62% purity) as a colorless oil. LCMS: (M+1: 607.0). This crude was used directly in the next alkylation reaction.

Step 6. To a solution of (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 47, (190 mg, 447 mol, 1 eq) and 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane (325 mg, 537 mol, 1.2 eq) in DMF (2 mL) was added K₂CO₃ (186 mg, 1.34 mmol, 3 eq). The mixture was stirred at 80° C. for 1 h. On completion, the reaction mixture was quenched by addition H₂O (8 mL) at 25° C. and extracted with EA (8 mL×3). The combined organic layers were washed with H₂O (10 mL×2), dried over Na₂SO₄, filtered, and concentrated under reduced pressure to give a residue. The residue was purified by CombiFlash (4 g silica gel column, THE in PE from 0-100%) to give (2S)-N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (340 mg, 358 μmol, 80% yield) as a brown oil. LCMS: (M+1: 949.7).

Step 7. A mixture of (2S)-N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (330 mg, 348 μmol, 1 eq), TBAC (96.6 mg, 348 mol, 97.2 μL, 1 eq), NaHCO₃ (73.0 mg, 869 μmol, 33.8 μL, 2.5 eq) and Pd(OAc)₂ (15.6 mg, 69.5 μmol, 0.2 eq) in DMF (16.5 mL) was degassed and purged with N₂ for 3 times, and then the mixture was stirred at 130° C. for 1.5 h under N₂ atmosphere. On completion, the reaction mixture was quenched by addition H₂O (30 mL) at 25° C. and extracted with EA (15 mL×3). The combined organic layers were washed with H₂O (20 mL×2), dried over Na₂SO₄, filtered, and concentrated under reduced pressure to give a residue. The residue was purified by CombiFlash (12 g silica gel column, THF in PE from 0-100%) to give (10S,17E)-14-[(2S)-1-{[tert-butyl(dimethyl)silyl]oxy}propan-2-yl]-6,8,10,12,20-pentamethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (275 mg, 335 μmol, 96% yield) as a brown oil. LCMS: (M+1: 821.8).

Step 8. To a solution of (10S,17E)-14-[(2S)-1-{[tert-butyl(dimethyl)silyl]oxy}propan-2-yl]-6,8,10,12,20-pentamethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (260 mg, 317 mol, 1 eq) in THE (2.6 mL) was added TBAF (1 M, 1.90 mL, 6 eq). The mixture was stirred at 70° C. for 3 hr. On completion, the reaction mixture was quenched by addition H₂O (10 mL) at 25° C. and extracted with EA (8 mL×3). The combined organic layers were washed with H₂O (10 mL×2), dried over Na₂SO₄, filtered and concentrated under reduced pressure to give (10S,17E)-14-[(2S)-1-hydroxypropan-2-yl]-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (200 mg, 243 mol, 77% yield, 70% purity) as a brown oil. LCMS: (M+1: 577.2).

Step 9. To a solution of (10S,17E)-14-[(2S)-1-hydroxypropan-2-yl]-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (90.0 mg, 156 mol, 1 eq) in DMF (1 mL) was added K₂CO₃(43.1 mg, 312 μmol, 2 eq) and commercially available 2-iodoacetonitrile (31.3 mg, 187 μmol, 1.2 eq). The mixture was stirred at 80° C. for 1 h. On completion, the reaction mixture was quenched by addition H₂O (8 mL) at 25° C. and extracted with EA (5 mL×3). The combined organic layers were washed with H₂O (6 mL×2), dried over Na₂SO₄, filtered and concentrated under reduced pressure to give {[(10S,17E)-14-[(2S)-1-hydroxypropan-2-yl]-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (100 mg, 114 mol, 73% yield, 70% purity) as a brown oil. LCMS: (M+1: 616.2).

Step 10. To a solution of {[(10S,17E)-14-[(2S)-1-hydroxypropan-2-yl]-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (80.0 mg, 130 μmol, 1 eq) in DCM (3 mL) was added TFA (921 mg, 8.08 mmol, 0.6 mL, 62.2 eq). The mixture was stirred at 25° C. for 0.25 h. On completion, the mixture was filtered and concentrated under reduced pressure to give a residue. The residue was purified by prep-HPLC (column: Waters xbridge 150*25 mm 10 um; mobile phase: [water(NH4HCO3)-ACN]; gradient:26%-56% B over 10 min) to give Ex. 52 (5.78 mg, 10.87 mol, 8.37% yield) as a brown solid. LCMS: (M+1: 532.2). ¹H NMR for Ex. 52 is shown in the below NMR table.

### Preparation of {[(10S,17E)-14-[(2S)-1-hydroxypropan-2-yl]-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 53)

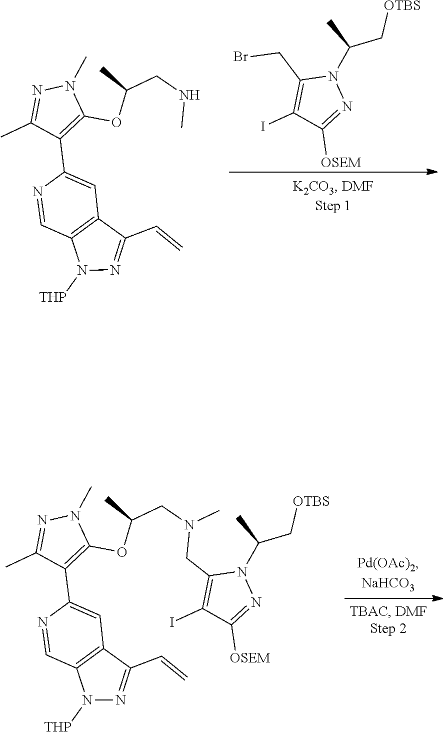

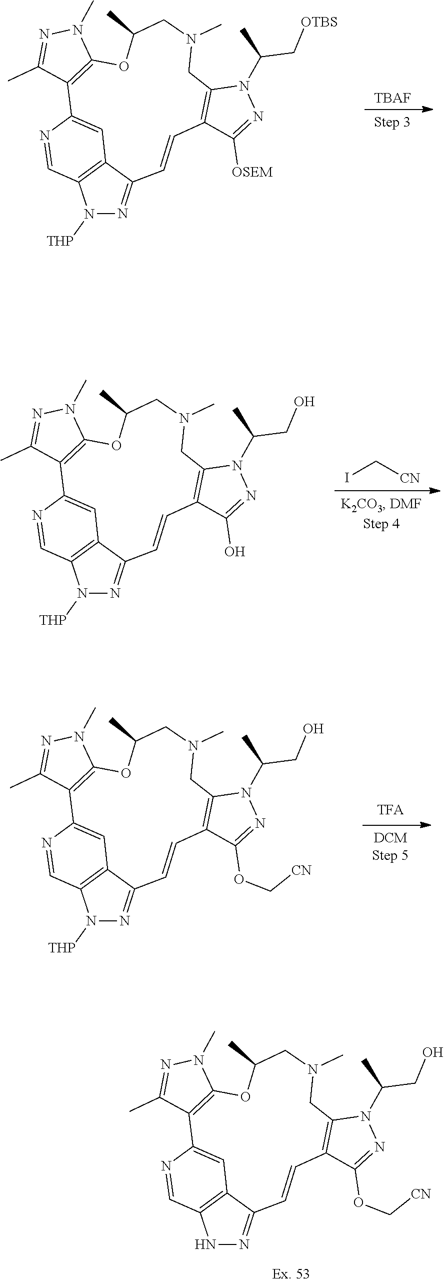

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (500 mg, 1.22 mmol, 1 eq), 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, (368 mg, 609 mol, 0.5 eq) in DMF (5 mL) was added K₂CO₃ (505 mg, 3.65 mmol, 3 eq). The mixture was stirred at 80° C. for 1 hr. On completion, the mixture was diluted with water (50 mL) and extracted with ethyl acetate (20 mL×3). The combined organic phase was washed with water (50 mL×2) and dried over Na₂SO₄, filtered and the filtrate was concentrated to give a residue. The residue was purified by column chromatography (SiO₂, Petroleum ether/Ethyl acetate=1:0 to 0:1) to give (2S)-N-[[2-[(1S)-2-[tert-butyl(dimethyl)silyl]oxy-1-methyl-ethyl]-4-iodo-5-(2-trimethylsilylethoxymethoxy) pyrazol-3-yl]methyl]-2-[2,5-dimethyl-4-(1-tetrahydropyran-2-yl-3-vinyl-pyrazolo [3,4-c]pyridin-5-yl)pyrazol-3-yl]oxy-N-methyl-propan-1-amine (225 mg, 240 μmol, 20% yield) as yellow oil. LCMS: (M+1: 935.2).

Step 2. A mixture of (2S)-N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (210 mg, 224 mol, 1 eq), TBAC (62.4 mg, 224 μmol, 1 eq), NaHCO₃ (47.1 mg, 561 μmol, 2.5 eq) and Pd(OAc)₂ (10.0 mg, 44.9 mol, 0.2 eq) in DMF (2 mL) was degassed and purged with N₂ for 3 times, and then the mixture was stirred at 80° C. for 2 h under N₂ atmosphere. On completion, the mixture was diluted with water (30 mL) and extracted with ethyl acetate (10 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:0 to 0:1) to give (10S,17E)-14-[(2S)-1-{[tert-butyl(dimethyl)silyl]oxy}propan-2-yl]-6,8,10,12-tetramethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f3′,4′-j:4″,3″-n][1,4]oxazacyclopentadecine (110 mg, 136 mol, 61% yield) as yellow oil. LCMS: (M+1: 807.3).

Step 3. To a solution of (10S,17E)-14-[(2S)-1-{[tert-butyl(dimethyl)silyl]oxy}propan-2-yl]-6,8,10,12-tetramethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (50.0 mg, 61.9 μmol, 1 eq) in TBAF (1 M, 0.5 mL) was stirred at 70° C. for 2 h. On completion, the mixture was diluted with water (10 mL) and extracted with ethyl acetate (8 mL×3). The combined organic phase dried over Na₂SO₄, filtered and the filtrate was concentrated to give (10S,17E)-14-[(2S)-1-hydroxypropan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (34.0 mg, crude) as yellow oil. LCMS: (M+1: 563.2).

Step 4. To a solution of (10S,17E)-14-[(2S)-1-hydroxypropan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (23.5 mg, 41.7 μmol, 1 eq), commercially available 2-iodoacetonitrile (8.37 mg, 50.1 μmol, 1.2 eq) in DMF (0.5 mL) was added K₂CO₃ (17.3 mg, 125 μmol, 3 eq). The mixture was stirred at 80° C. for 1 h. On completion, the mixture was diluted with water (50 mL) and extracted with ethyl acetate (20 mL×3). The combined organic phase was washed with water (20 mL×2) and dried over Na₂SO₄, filtered and the filtrate was concentrated to give {[(10S,17E)-14-[(2S)-1-hydroxypropan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (25.0 mg, crude) as yellow oil. LCMS: (M+1: 602.2).

Step 5. To a solution of {[(10S,17E)-14-[(2S)-1-hydroxypropan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (20.0 mg, 33.2 mol, 1 eq) in DCM (1 mL) was added TFA (2.69 mmol, 0.2 mL, 81 eq). The mixture was stirred at 25° C. for 1 h. On completion, the mixture was concentrated to give a residue. The residue was purified by prep-HPLC purification (Phenomenex luna C18 150*25 mm*10 um; mobile phase: [water(FA)-ACN]; gradient:16%-46% B over 15 min) to give Ex. 53 (3.30 mg, 6.38 μmol, 19.18% yield) as brown solid. LCMS: (M+1: 518.1). ¹H NMR for Ex. 53 is shown in the below NMR table.

### Preparation of (2S)-2-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 54)

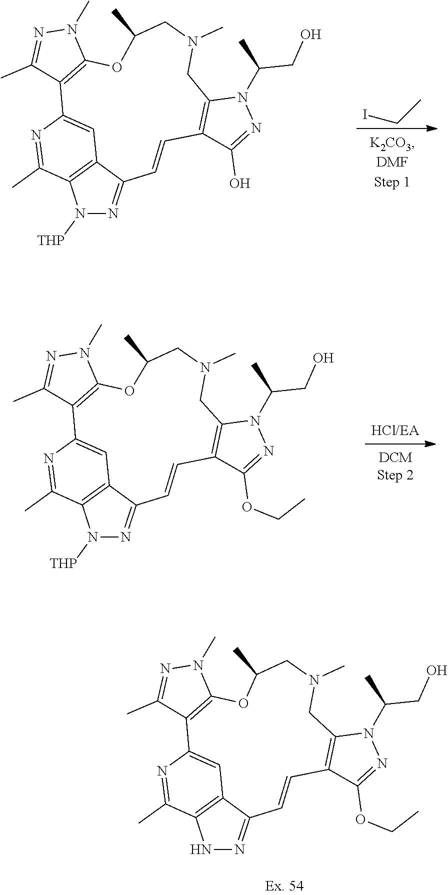

Step 1. To a solution of (10S,17E)-14-[(2S)-1-hydroxypropan-2-yl]-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (90.0 mg, 156 mol, 1 eq), which was prepared according to steps 1-8 of Ex. 52, in DMF (2 mL) was added K₂CO₃ (43.1 mg, 312 μmol, 2 eq) and iodoethane (29.2 mg, 187 mol, 15.0 μL, 1.2 eq). The mixture was stirred at 80° C. for 1 h. On completion, the reaction mixture was quenched by addition H₂O (8 mL) at 25° C., and extracted with EA (5 mL×3). The combined organic layers were washed with H₂O (6 mL×2), dried over Na₂SO₄, filtered and concentrated under reduced pressure to give (2S)-2-[(10S,17E)-16-ethoxy-6,8,10,12,20-pentamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (90.0 mg, 149 μmol, 95% yield) as a brown oil. LCMS: (M+1: 605.3).

Step 2. To a solution of (2S)-2-[(10S,17E)-16-ethoxy-6,8,10,12,20-pentamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (80.0 mg, 132 mol, 1 eq) in DCM (2 mL) was added HCl/EtOAc (4 M, 1 mL, 30.2 eq). The mixture was stirred at 25° C. for 0.25 h. On completion, the mixture was filtered and concentrated under reduced pressure to give a residue. The residue was purified by prep-HPLC (column: Phenomenex luna C18 150*25 mm*10 um; mobile phase: [water(FA)-ACN]; gradient:16%-46% B over 10 min) to give Ex. 54 (4.63 mg, 7.96 μmol, 6.02% yield, 97.42% purity, FA) as a yellow solid. LCMS: (M+1: 521.2). ¹H NMR for Ex. 54 is shown in the below NMR table.

### Preparation of (2S)-2-[(8aR,9S,19E)-1-ethoxy-9,11,13-trimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]propan-1-ol (Ex. 56)

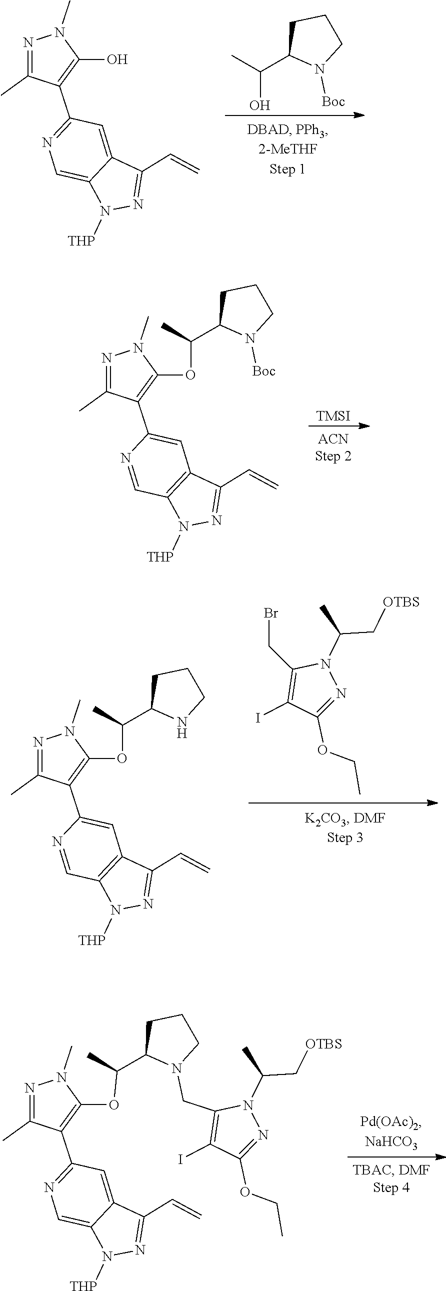

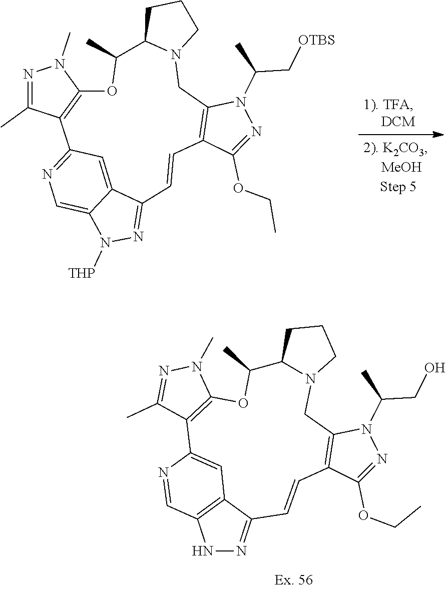

Step 1. A mixture of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (2.00 g, 5.89 mmol, 1 eq), tert-butyl (2R)-2-(1-hydroxyethyl)pyrrolidine-1-carboxylate, which was prepared according to the method described in Ex. 32/Ex. 33, (2.54 g, 11.8 mmol, 2 eq), DBAD (2.99 g, 13.0 mmol, 2.2 eq), PPh₃ (3.40 g, 13.0 mmol, 2.2 eq) was degassed and purged with N₂ for 3 times, and then the mixture was added 2-MeTHF (20 mL), degassed and purged with N₂ again. Then the mixture was stirred at 25° C. for 2 hr under N₂ atmosphere. On completion, the mixture was concentrated in vacuum to give a residue. The residue was purified by column chromatography (SiO₂, Petroleum ether/THF=7/3) to give tert-butyl (2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidine-1-carboxylate (1.24 g, 2.31 mmol, 39% yield) as a red oil. LCMS: (M+1=537.1).

Step 2. To a solution of tert-butyl (2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidine-1-carboxylate (1.23 g, 2.29 mmol, 1 eq) in ACN (12 mL) was added TMSI (596 mg, 2.98 mmol, 1.3 eq) dropwise at 0° C. The mixture was stirred at 0° C. for 30 min. On completion, the reaction mixture was adjusted to pH=8 with saturated solution of NaHCO₃ and partitioned between ethyl acetate (30 mL×3) and water (100 mL). The combined organic phase was washed with brine (50 mL), dried over anhydrous sodium sulfate, filtered and concentrated to give a residue. The residue was purified by column chromatography (SiO₂, DCM/MeOH=83:17) to give 5-[1,3-dimethyl-5-[1-[(2R)-pyrrolidin-2-yl]ethoxy]pyrazol-4-yl]-1-tetrahydropyran-2-yl-3-vinyl-pyrazolo[3,4-c]pyridine (800 mg, 1.83 mmol, 80% yield) as a pink solid. LCMS: (M+1=437.1).

Step 3. To a solution of 5-[1,3-dimethyl-5-[1-[(2R)-pyrrolidin-2-yl]ethoxy]pyrazol-4-yl]-1-tetrahydropyran-2-yl-3-vinyl-pyrazolo[3,4-c]pyridine (300 mg, 687 μmol, 1 eq) and [(2S)-2-[5-(bromomethyl)-3-ethoxy-4-iodo-pyrazol-1-yl]propoxy]-tert-butyl-dimethyl-silane, which was prepared according to the method described in Ex. 19, (346 mg, 687 mol, 1 eq) in DMF (3 mL) was added K₂CO₃ (190 mg, 1.37 mmol, 2 eq). The mixture was stirred at 80° C. for 1.5 hr. On completion, the reaction mixture was partitioned between ethyl acetate (25 mL×3) and water (80 mL). The combined organic phase was washed with water (50 mL), dried over anhydrous sodium sulfate, filtered and concentrated to give a residue. The residue was purified by column chromatography (SiO₂, Petroleum ether/Tetrahydrofuran=73:27) to give tert-butyl-[(2S)-2-[5-[[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]methyl]-3-ethoxy-4-iodo-pyrazol-1-yl]propoxy]-dimethyl-silane (400 mg, 466 mol, 68% yield) as a light-yellow solid. LCMS: (M+1=859.1).

Step 4. A mixture of tert-butyl-[(2S)-2-[5-[[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]methyl]-3-ethoxy-4-iodo-pyrazol-1-yl]propoxy]-dimethyl-silane (360 mg, 419 mol, 1 eq), TBAC (116 mg, 419 mol, 1 eq), NaHCO₃ (88.0 mg, 1.05 mmol, 2.5 eq) and Pd(OAc)₂ (18.8 mg, 83.8 mol, 0.2 eq) in DMF (4 mL) was degassed and purged with N₂ for 3 times, and then the mixture was stirred at 130° C. for 50 min under N₂ atmosphere. On completion, the reaction mixture was partitioned between ethyl acetate (6 mL×3) and water (20 mL). The combined organic phase was washed with water (20 mL), dried over anhydrous sodium sulfate, filtered and concentrated to give (8aR,9S,19E)-3-[(2S)-1-{[tert-butyl(dimethyl)silyl]oxy}propan-2-yl]-1-ethoxy-9,11,13-trimethyl-17-(oxan-2-yl)-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecine (430 mg, 241 mol, 58% yield, 41% purity) as a black brown solid. LCMS: (M+1=731.2).

Step 5. To a solution of (8aR,9S,19E)-3-[(2S)-1-{[tert-butyl(dimethyl)silyl]oxy}propan-2-yl]-1-ethoxy-9,11,13-trimethyl-17-(oxan-2-yl)-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecine (390 mg, 534 μmol, 1 eq) in DCM (4 mL) was added TFA (20.2 mmol, 1.5 mL, 37.85 eq). The mixture was stirred at 25° C. for 2.5 hr. On completion, the mixture was concentrated in vacuum to give a brown oil (300 mg, 465 μmol, 87% yield). LCMS: (M+1=629.1).

This brown oil (254 mg, 395 μmol, 1 eq) was dissolved in MeOH (0.3 mL) and K₂CO₃(218 mg, 1.58 mmol, 4 eq) was added. The mixture was stirred at 25° C. for 20 min. On completion, the mixture was filtered. The filtrate was purified by prep-HPLC purification (column: Welch Ultimate C18 150*25 mm*5 um; mobile phase: [water(FA)-ACN]; gradient:9%-39% B over 11 min) to give Ex. 56 (18.32 mg, 34.39 μmol, 8.70% yield) as a yellow solid. LCMS: (M+1=533.2). ¹H NMR for Ex. 56 is shown in the below NMR table.

### Preparation of (2S)-2-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 57)

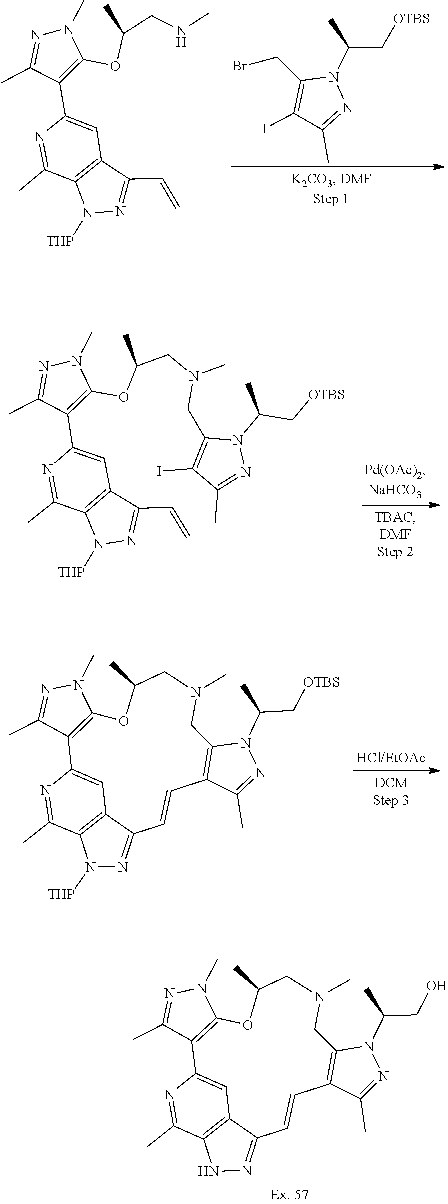

Step 1. To a solution of [(2S)-2-[5-(bromomethyl)-4-iodo-3-methyl-pyrazol-1-yl]propoxy]-tert-butyl-dimethyl-silane, which was prepared according to the method described in Ex. 46, (200 mg, 424 mol, 1.2 eq) and (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 47, (150 mg, 353 mol, 1 eq) in DMF (3 mL) was added K₂CO₃ (146 mg, 1.06 mmol, 3 eq). The mixture was stirred at 80° C. for 1 h. On completion, the reaction mixture was quenched by addition of H₂O (30 mL) at 25° C. and extracted with EA (15 mL×3). The combined organic layers were washed with H₂O (50 mL×2), dried over Na₂SO₄, filtered, and concentrated under reduced pressure to give a residue. The residue was purified by CombiFlash (4 g silica gel column, THE in PE from 0-100%) to give (2S)-N-[[2-[(1S)-2-[tert-butyl(dimethyl)silyl]oxy-1-methyl-ethyl]-4-iodo-5-methyl-pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (240 mg, 293 mol, 83% yield) as a brown oil. LCMS: (M+1:817.5).

Step 2. A mixture of (2S)-N-[[2-[(1S)-2-[tert-butyl(dimethyl)silyl]oxy-1-methyl-ethyl]-4-iodo-5-methyl-pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (230 mg, 281 μmol, 1 eq), TBAC (78.2 mg, 282 μmol, 1 eq), NaHCO₃ (59.1 mg, 704 μmol, 2.5 eq) and Pd(OAc)₂ (12.6 mg, 56.3 mol, 0.2 eq) in DMF (3 mL) was degassed and purged with N₂ for 3 times, and then the mixture was stirred at 130° C. for 1 h under N₂ atmosphere. On completion, the reaction mixture was quenched by addition of H₂O (30 mL) at 25° C. and extracted with EA (15 mL×3). The combined organic layers were washed with H₂O (50 mL×2), dried over Na₂SO₄, filtered and concentrated under reduced pressure to give (10S,17E)-14-[(2S)-1-f[tert-butyl(dimethyl)silyl]oxy}propan-2-yl]-6,8,10,12,16,20-hexamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (160 mg, 232 μmol, 82% yield) as a brown oil. LCMS: (M+1:689.8).

Step 3. To a solution of (10S,17E)-14-[(2S)-1-{[tert-butyl(dimethyl)silyl]oxy}propan-2-yl]-6,8,10,12,16,20-hexamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (150 mg, 218 μmol, 1 eq) in DCM (3 mL) was added HCl/EtOAc (4 M, 3 mL, 55.1 eq). The mixture was stirred at 25° C. for 0.5 h. On completion, the mixture was filtered and concentrated under reduced pressure to give a residue. The residue was purified by prep-HPLC (column: Phenomenex Luna C18 150*25 mm*10 um; mobile phase: [water(FA)-ACN]; gradient:4%-34% B over 10 min) to give Ex. 57 (44.94 mg, 90.51 mol, 41.57% yield, 98.81% purity) as a yellow solid. LCMS: (M+1: 491.2). ¹H NMR for Ex. 57 is shown in the below NMR table.

### Preparation of (2S)-2-[(17E)-16-ethoxy-6,8,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 59)

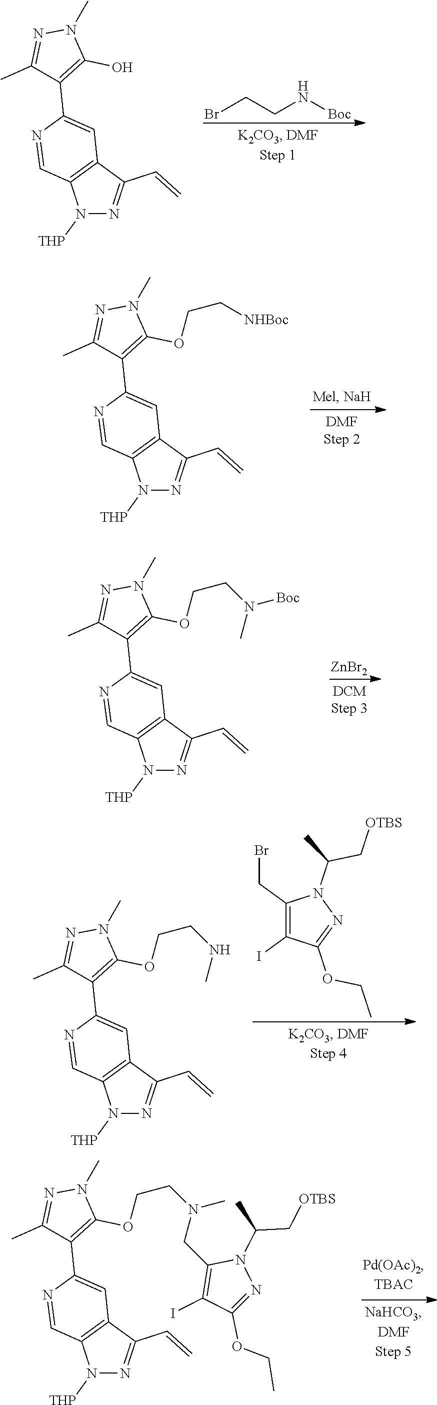

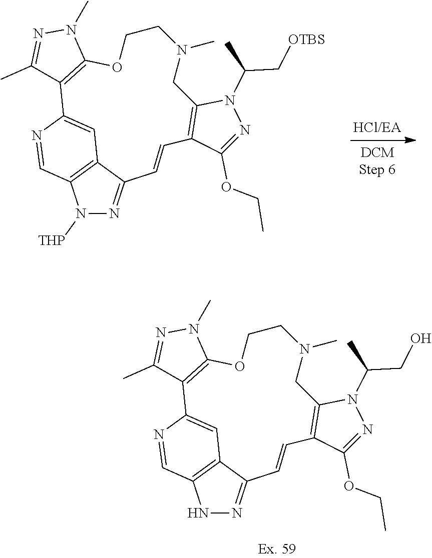

Step 1. To a solution of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (1.50 g, 4.42 mmol, 1 eq) in DMF (15 mL) was added K₂CO₃ (1.83 g, 13.3 mmol, 3 eq) and commercially available tert-butyl N-(2-bromoethyl)carbamate (1.29 g, 5.75 mmol, 1.3 eq). The mixture was stirred at 80° C. for 2 h. On completion, the reaction mixture was diluted with H₂O (30 mL) and extracted with EA (50 mL×3). The combined organic layers were dried over Na₂SO₄, filtered and concentrated under reduced pressure to give a residue. The residue was purified by column chromatography (SiO₂ Petroleum ether/Ethyl acetate=1/0 to 0/1) to give tert-butyl N-[2-[2,5-dimethyl-4-(1-tetrahydropyran-2-yl-3-vinyl-pyrazolo[3,4-c]pyridin-5-yl)pyrazol-3-yl]oxyethyl]carbamate (1.18 g, 2.45 mmol, 55% yield) as red oil. ¹H NMR (400 MHz, CDCl₃) S=9.26-9.17 (m, 1H), 7.87-7.80 (m, 1H), 7.11-6.99 (m, 1H), 6.16-6.03 (m, 1H), 5.85-5.76 (m, 2H), 5.64-5.56 (m, 1H), 4.08-4.05 (m, 2H), 3.84-3.75 (m, 1H), 3.74-3.72 (m, 3H), 3.45-3.39 (m, 2H), 2.60-2.48 (m, 1H), 2.38 (s, 3H), 2.19-2.10 (m, 2H), 1.87-1.66 (m, 4H), 1.46 (s, 9H). LCMS: (M+1=483.2).

Step 2. To a solution of tert-butyl N-[2-[2,5-dimethyl-4-(1-tetrahydropyran-2-yl-3-vinyl-pyrazolo[3,4-c]pyridin-5-yl)pyrazol-3-yl]oxyethyl]carbamate (1.08 g, 2.24 mmol, 1 eq) in DMF (10 mL) was added NaH (179 mg, 4.48 mmol, 60% purity, 2 eq) at 0° C. and stirred at 25° C. for 30 mines. And then MeI (476 mg, 3.36 mmol, 1.5 eq) was added at 0° C. and stirred at 25° C. for 1 h. On completion, the reaction mixture was quenched with sat. NH₄Cl (15 mL) at 0° C. The residue was purified by column chromatography (SiO₂, Petroleum ether/Ethyl acetate=1/0 to 0/1) to give tert-butyl N-[2-[2,5-dimethyl-4-(1-tetrahydropyran-2-yl-3-vinyl-pyrazolo[3,4-c]pyridin-5-yl)pyrazol-3-yl]oxyethyl]-N-methyl-carbamate (960 mg, 1.93 mmol, 86% yield) as yellow oil. LCMS: (M+1=497.3).

Step 3. To a solution of tert-butyl N-[2-[2,5-dimethyl-4-(1-tetrahydropyran-2-yl-3-vinyl-pyrazolo[3,4-c]pyridin-5-yl)pyrazol-3-yl]oxyethyl]-N-methyl-carbamate (860 mg, 1.73 mmol, 1 eq) in DCM (8 mL) was added ZnBr₂ (1.17 g, 5.20 mmol, 3 eq). The mixture was stirred at 25° C. for 8 h. On completion, the reaction mixture was concentrated under reduced pressure to give 2-[2,5-dimethyl-4-(1-tetrahydropyran-2-yl-3-vinyl-pyrazolo[3,4-c]pyridin-5-yl)pyrazol-3-yl]oxy-N-methyl-ethanamine (2.10 g, 1.59 mmol, 92% yield, 30% purity) as gray solid. LCMS: (M+1=397.1).

Step 4. To a solution of 2-[2,5-dimethyl-4-(1-tetrahydropyran-2-yl-3-vinyl-pyrazolo|3,4-c|pyridin-5-yl)pyrazol-3-yl]oxy-N-methyl-ethanamine (1.90 g, 4.79 mmol, 1 eq) in DMF (15 mL) was added K₂CO₃ (1.99 g, 14.4 mmol, 3 eq) and [(2S)-2-[5-(bromomethyl)-3-ethoxy-4-iodo-pyrazol-1-yl]propoxy]-tert-butyl-dimethyl-silane, which was prepared according to the method described in Ex. 19, (1.45 g, 2.88 mmol, 0.6 eq). The mixture was stirred at 80° C. for 2 h. On completion, the reaction mixture was diluted with H₂O (20 mL) and extracted with EA (40 mL×3). The combined organic layers were dried over Na₂SO₄, filtered and concentrated under reduced pressure to give a residue. The residue was purified by column chromatography (SiO₂, Petroleum ether/Ethyl acetate=1/0 to 0/1) to give N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-ethanamine (760 mg, 928 μmol, 19% yield) as yellow oil. LCMS: (M+1=819.2).

Step 5. A mixture of N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-ethanamine (700 mg, 855 μmol, 1 eq), Pd(OAc)₂ (38.4 mg, 171 μmol, 0.2 eq), NaHCO₃ (180 mg, 2.14 mmol, 2.50 eq) and TBAC (238 mg, 855 mol, 1 eq) in DMF (7 mL) was degassed and purged with N₂ for 3 times, and then the mixture was stirred at 140° C. for 1 h under N₂ atmosphere. On completion, the reaction mixture was diluted with H₂O (14 mL) and extracted with EA (30 mL×3). The combined organic layers were dried over Na₂SO₄, filtered and concentrated under reduced pressure to give a residue. The residue was purified by column chromatography (SiO₂, Petroleum ether/EA=1/0 to 0/1) to give (17E)-14-[(2S)-1-{[tert-butyl(dimethyl)silyl]oxy}propan-2-yl]-16-ethoxy-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (530 mg, 767 μmol, 90% yield) as yellow oil. LCMS: (M+1=691.2).

Step 6. To a solution of (17E)-14-[(2S)-1-{[tert-butyl(dimethyl)silyl]oxy}propan-2-yl]-16-ethoxy-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (315 mg, 456 μmol, 1 eq) in DCM (3.6 mL) was added HCl/EtOAc (4.00 M, 1.80 mL, 15.8 eq) at 0° C. The mixture was stirred at 25° C. for 2 h. On completion, the reaction mixture was concentrated under reduced pressure to remove solvent. The residue was purified by prep-HPLC (column: Welch Ultimate C18 150*25 mm*5 um; mobile phase: [water(FA)-ACN]; gradient:24%-54% B over 10 min) to give Ex. 59 (67.05 mg, 136.12 μmol, 29.86% yield, 100% purity) as orange solid. LCMS: (M+1=493.1). ¹H NMR for Ex. 59 is shown in the below NMR table.

### Preparation of 2-[(10S,17E)-16-ethyl-6,8,10,12-tetramethyl-2,8,10,11,12,13-hexahydro-14H-5,3-(azenometheno)tripyrazolol3,4-f:3′,4′-j:4″,3″-n][1,4]oxazacyclopentadecin-14-yl]ethan-1-ol (Ex. 60)

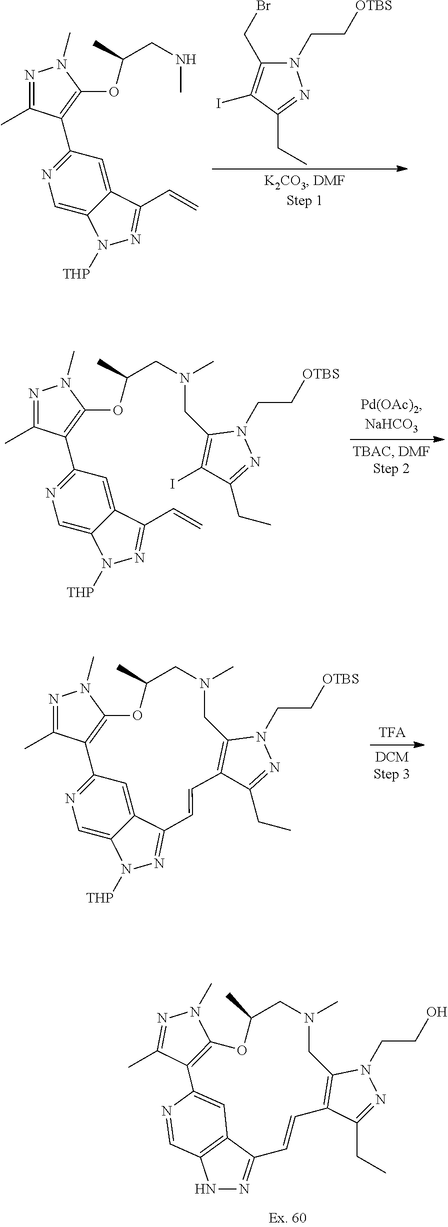

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (300 mg, 730 μmol, 1 eq), 2-[5-(bromomethyl)-3-ethyl-4-iodo-pyrazol-1-yl]ethoxy-tert-butyl-dimethyl-silane, which was prepared according to the method described in Ex. 55, (345 mg, 730 μmol, 1 eq) in DMF (5 mL) was added K₂CO₃(303 mg, 2.19 mmol, 3 eq). The mixture was stirred at 80° C. for 1 h. On completion, the mixture was diluted with water (50 mL) and extracted with ethyl acetate (20 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:0 to 0:1) to give (2S)-N-[[2-[2-[tert-butyl(dimethyl)silyl]oxyethyl]-5-ethyl-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (210 mg, 261 mol, 36% yield) as yellow oil. LCMS: (M+1: 803.1).

Step 2. A mixture of (2S)-N-[[2-[2-[tert-butyl(dimethyl)silyl]oxyethyl]-5-ethyl-4-iodo-pyrazol-3-yl]methyl]-2-[2,5-dimethyl-4-(1-tetrahydropyran-2-yl-3-vinyl-pyrazolo [3,4-c]pyridin-5-yl)pyrazol-3-yl]oxy-N-methyl-propan-1-amine (200 mg, 249 μmol, 1 eq), TBAC (69.2 mg, 249 mol, 1 eq), diacetoxypalladium (11.1 mg, 49.8 mol, 0.2 eq) and NaHCO₃ (52.3 mg, 622 mol, 2.5 eq) in DMF (2 mL) was degassed and purged with N₂ for 3 times, and then the mixture was stirred at 80° C. for 1 h under N₂ atmosphere. On completion, the mixture was diluted with water (50 mL) and extracted with ethyl acetate (20 mL×3). The combined organic phase was washed with water (50 mL×2) and dried over Na₂SO₄, filtered and the filtrate was concentrated to give a residue. The residue was purified by column chromatography (SiO₂, Petroleum ether/Ethyl acetate=1:0 to 1:1) to give (10S,17E)-14-(2-{[tert-butyl(dimethyl)silyl]oxy}ethyl)-16-ethyl-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (100 mg, 148 mol, 59% yield) as yellow oil. LCMS: (M+1: 675.2).

Step 3. To a solution of (10S,17E)-14-(2-{[tert-butyl(dimethyl)silyl]oxy}ethyl)-16-ethyl-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (80.0 mg, 118 μmol, 1 eq) in DCM (3 mL) was added TFA (13.5 mmol, 1.00 mL, 114 eq). The mixture was stirred at 25° C. for 1 h. On completion, the mixture was filtered and concentrated to give a residue. The residue was purified by prep-HPLC purification (Phenomenex luna C18 150*25 mm*1Oum; mobile phase: [water(FA)-ACN]; gradient:1%-31% B over 15 min) to give Ex. 60 (1.95 mg, 4.09 μmol, 3.45% yield) as off-white solid. LCMS: (M+1: 477.1). ¹H NMR for Ex. 60 is shown in the below NMR table.

### Preparation of (2S)-2-[(10S,17E)-16-ethyl-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 61)

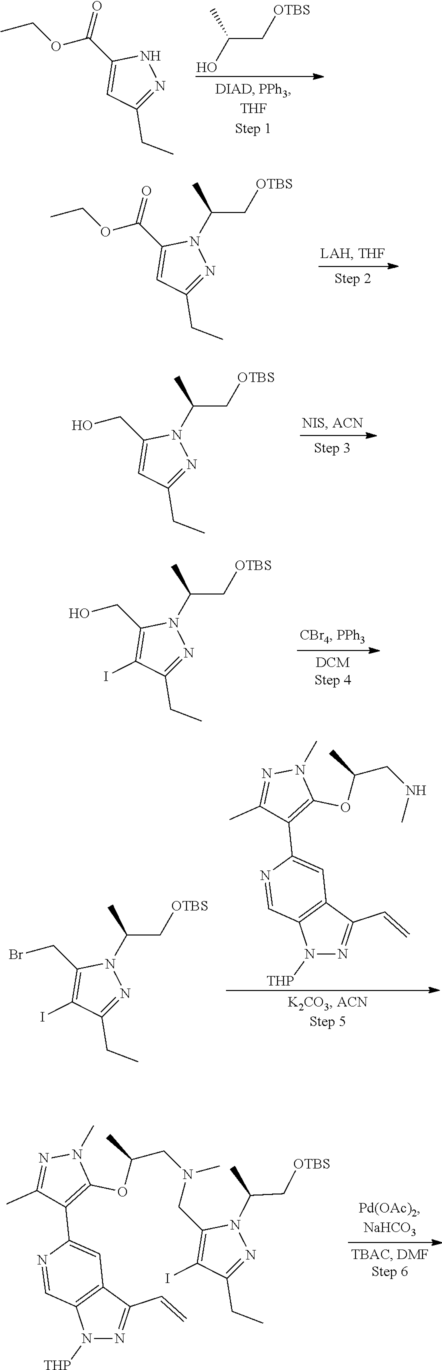

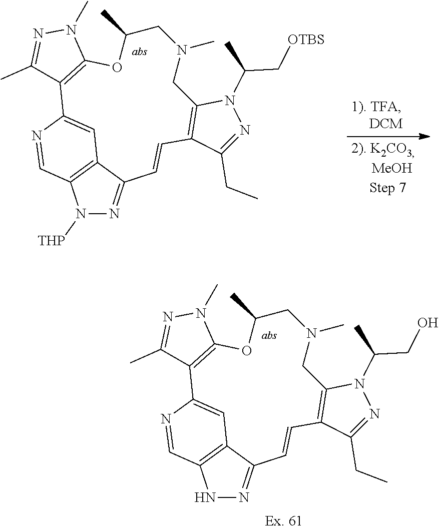

Step 1. To a solution of ethyl 3-ethyl-1H-pyrazole-5-carboxylate, which was prepared according to the method described in Ex. 55, (9.70 g, 57.6 mmol, 1 eq) and (2R)-1-[tert-butyl(dimethyl)silyl]oxypropan-2-ol, which was prepared according to the method described in Ex. 19, (21.9 g, 115 mmol, 2 eq) in THE (100 mL) was added DIAD (25.6 g, 126 mmol, 2.2 eq) and PPh₃ (33.2 g, 126 mmol, 2.2 eq). The mixture was stirred at 0° C. for 2 h. On completion, the mixture was concentrated to give a residue. The residue was purified by column chromatography (SiO₂, PE/THF=2:1 to 1:1) to give ethyl 2-[(1S)-2-[tert-butyl(dimethyl)silyl]oxy-1-methyl-ethyl]-5-ethyl-pyrazole-3-carboxylate (24.0 g, 56.3 mmol, 97% yield, 80% purity) as a yellow oil. LCMS: (M+1: 341.2).

Step 2. To a solution of ethyl 2-[(1R)-2-[tert-butyl(dimethyl)silyl]oxy-1-methyl-ethyl]-5-ethyl-pyrazole-3-carboxylate (10.0 g, 29.3 mmol, 1 eq) in THF (100 mL) was added LiAlH₄ (2.5 M, 15 mL, 1.3 eq) at 0° C. The mixture was stirred at 0° C. for 2 h. On completion, the mixture was quenched with water (200 mL) and extracted with EA (50 mL×3). The combined organic phase was dried over anhydrous sodium sulfate, filtered and concentrated to give a residue. The residue was purified by column chromatography (SiO₂, PE/THF=5:1 to 5:1) to give [2-[(1R)-2-[tert-butyl(dimethyl)silyl]oxy-1-methyl-ethyl]-5-ethyl-pyrazol-3-yl]methanol (6.70 g, 22.4 mmol, 76% yield) as a yellow oil. LCMS: (M+1:299.1).

Step 3. To a solution of [2-[(1S)-2-[tert-butyl(dimethyl)silyl]oxy-1-methyl-ethyl]-5-ethyl-pyrazol-3-yl]methanol (5.30 g, 17.7 mmol, 1 eq) in ACN (50 mL) was added NIS (3.99 g, 17.7 mmol, 1 eq). The mixture was stirred at 60° C. for 2 h. On completion, the mixture was quenched with water (60 mL) and extracted with ethyl acetate (75 mL×3). The combined organic phase was dried over anhydrous sodium sulfate, filtered and concentrated to give a residue. The residue was purified by column chromatography (SiO₂, PE/THF=5:1 to 4:1) to give [2-[(1S)-2-[tert-butyl(dimethyl)silyl]oxy-1-methyl-ethyl]-5-ethyl-4-iodo-pyrazol-3-yl]methanol (6.60 g, 15.5 mmol, 87% yield) as a colourless oil. LCMS: (M+1:425.0).

Step 4. To a solution of [2-[(1S)-2-[tert-butyl(dimethyl)silyl]oxy-1-methyl-ethyl]-5-ethyl-4-iodo-pyrazol-3-yl]methanol (6.50 g, 15.3 mmol, 1 eq) in DCM (1 mL) was added PPh₃ (4.82 g, 18.3 mmol, 1.2 eq) and CBr₄ (6.10 g, 18.3 mmol, 1.2 eq). The mixture was stirred at 0° C. for 2 h. On completion, the mixture was filtered and concentrated to give a residue. The residue was purified by column chromatography (SiO₂, Petroleum ether/Ethyl acetate=6:1 to 6:1) to give |(2S)-2-15-(bromomethyl)-3-ethyl-4-iodo-pyrazol-1-yl|propoxyl-tert-butyl-dimethyl-silane (6.10 g, 12.5 mmol, 82% yield) as a colourless oil. LCMS: (M+1:488.9).

Step 5. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, (200 mg, 487 mol, 1 eq) and [(2S)-2-[5-(bromomethyl)-3-ethyl-4-iodo-pyrazol-1-yl]propoxy]-tert-butyl-dimethyl-silane (237 mg, 487 μmol, 1 eq) in ACN (6 mL) was added K₂CO₃ (202 mg, 1.46 mmol, 3 eq). The mixture was stirred at 60° C. for 2 h. On completion, the mixture was filtered and concentrated to give a residue. The residue was purified by column chromatography (SiO₂, Petroleum ether/Ethyl acetate=1:1 to 1:1) to give (2S)-N-[[2-[(1S)-2-[tert-butyl(dimethyl)silyl]oxy-1-methyl-ethyl]-5-ethyl-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (440 mg, 355 mol, 73% yield, 66% purity) as a yellow solid. LCMS: (M+1:817.4).

Step 6. To a solution of (2S)-N-[[2-[(1S)-2-[tert-butyl(dimethyl)silyl]oxy-1-methyl-ethyl]-5-ethyl-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (400 mg, 489 μmol, 1 eq) in DMF (20 mL) was added NaHCO₃ (102 mg, 1.22 mmol, 2.5 eq), TBAC (136 mg, 489 μmol, 1 eq) and Pd(OAc)₂ (32.9 mg, 146 μmol, 0.3 eq). The mixture was stirred at 130° C. for 2 h under N₂. On completion, the mixture was quenched with water (40 mL) and extracted with EA (25 mL×3). The combined organic phase was dried over anhydrous sodium sulfate, filtered and concentrated to give a residue. The residue was purified by column chromatography (SiO₂, PE/THF=1:1 to 1:1) to give (10S,17E)-14-[(2S)-1-{[tert-butyl(dimethyl)silyl]oxy}propan-2-yl]-16-ethyl-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (260 mg, 377 mol, 77% yield) as a white solid. LCMS: (M+1:689.5).

Step 7. To a solution of (10S,17E)-14-[(2S)-1-{[tert-butyl(dimethyl)silyl]oxy}propan-2-yl]-16-ethyl-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (200 mg, 290 μmol, 1 eq) in DCM (0.5 mL) was added TFA (7.67 g, 67.3 mmol, 231 eq). The mixture was stirred at 25° C. for 2 h. On completion, the mixture was concentrated to give a yellow oil (170 mg, 289 μmol, 99% yield). LCMS: (M+1:587.2).

This brown oil (170 mg, 289 mol, 1 eq) was dissolved in MeOH (1 mL) and K₂CO₃ (200 mg, 1.45 mmol, 5 eq) was added. The mixture was stirred at 25° C. for 2 h. On completion, the mixture was filtered and concentrated to give a residue. The crude product was purified by prep-HPLC (column: Waters xbridge 150*25 mm 10 um; mobile phase: [water (NH₄HCO₃)-ACN]; gradient:24%-54% B over 10 min) to give Ex. 61 (39.04 mg, 79.58 μmol, 27.46% yield) as a white solid. LCMS: (M+1:491.2). ¹H NMR for Ex. 61 is shown in the below NMR table.

### Preparation of {[(10S,17E)-14-(2-hydroxyethyl)-6,8,10,12,20-pentamethyl-2,10,11,12,13,14-hexahydro-8H-5,3-(azenometheno)tripyrazolo[3,4-f:3,4′-j:4″,3″-n][1,4]oxazacyclopentadecin-16-yl]oxy}acetonitrile (Ex. 63)

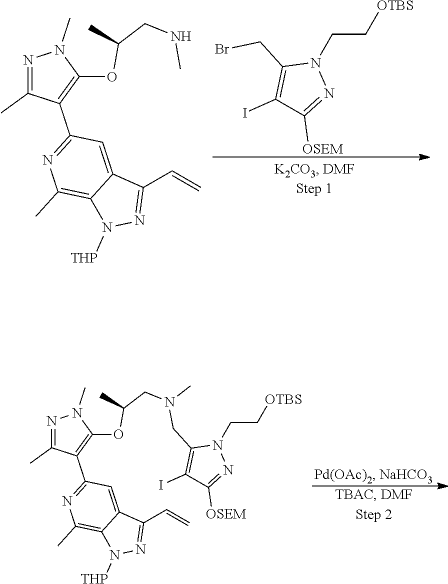

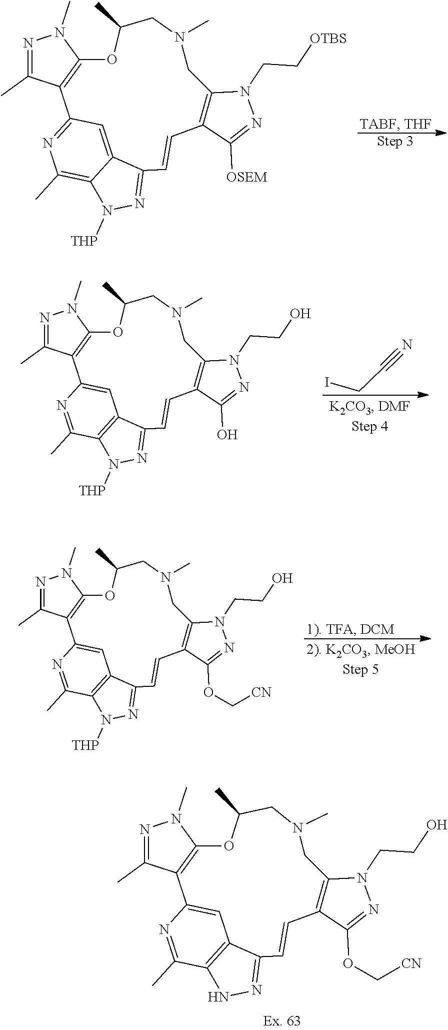

Step 1. To a solution of (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 47, (290 mg, 683 mol, 1 eq) in DMF (3 mL) was added 2-[[5-(bromomethyl)-1-[2-[tert-butyl(dimethyl)silyl]oxyethyl]-4-iodo-pyrazol-3-yl]oxymethoxy]ethyl-trimethyl-silane, which was prepared according to the method described in Ex. 42, (444 mg, 751 mol, 1.1 eq) and K₂CO₃ (283 mg, 2.05 mmol, 3 eq). The mixture was stirred at 80° C. for 2 h. On completion, the reaction mixture was diluted with H₂O (15 mL) and extracted with EA (10 mL×3). The combined organic layers were dried over Na₂SO₄, filtered and concentrated under reduced pressure to give a residue. The residue was purified by column chromatography (SiO₂, Petroleum ether/THF=1:0 to 3:1) to give (2S)-N-[[2-[2-[tert-butyl(dimethyl) silyl]oxyethyl]-4-iodo-5-(2-trimethylsilylethoxymethoxy)pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (320 mg, 311 μmol, 46% yield, 91% purity) as an orange oil. LCMS: (M+1: 935.2).

Step 2. To a solution of (2S)-N-[[2-[2-[tert-butyl(dimethyl)silyl]oxyethyl]-4-iodo-5-(2-trimethylsilylethoxymethoxy)pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (270 mg, 289 μmol, 1 eq) in DMF (3 mL) was added NaHCO₃ (121 mg, 1.44 mmol, 5 eq) and TBAC (160 mg, 577 gmol, 2 eq) and Pd(OAc)₂ (9.72 mg, 43.3 μmol, 0.15 eq). The mixture was stirred at 130° C. for 1 h under N₂ atmosphere. On completion, the reaction mixture was diluted with H₂O (10 mL) and extracted with EA (8 mL×3). The combined organic layers were washed with H₂O (20 mL), dried over Na₂SO₄, filtered and concentrated under reduced pressure to give (10S,17E)-14-(2-{1[tert-butyl(dimethyl)silyl]oxy}ethyl)-6,8,10,12,20-pentamethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (230 mg, crude) as a black brown solid. LCMS: (M+1: 807.3).

Step 3. To a solution of (10S,17E)-14-(2-{[tert-butyl(dimethyl)silyl]oxy}ethyl)-6,8,10,12,20-pentamethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (100 mg, 124 μmol, 1 eq) in THF (1 mL) was added TBAF (1 M, 1 mL, 8 eq). The mixture was stirred at 70° C. for 16 h. On completion, the reaction mixture was diluted with H₂O (3 mL) and extracted with EA (3 mL×3). The combined organic layers were washed with H₂O (8 mL), dried over Na₂SO₄, filtered and concentrated under reduced pressure to give (10S,17E)-14-(2-hydroxyethyl)-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (150 mg, crude) as an orange oil. ¹H NMR (400 MHz, CDCl₃) δ=8.11 (d, J=8.4, 15.6 Hz, 1H), 7.99-7.92 (m, 1H), 7.44-7.35 (m, 1H), 5.87-5.77 (m, 1H), 4.95-4.83 (m, 1H), 4.15-4.09 (m, 2H), 3.99-3.89 (m, 2H), 3.68 (s, 3H), 3.39-3.30 (m, 5H), 2.90 (s, 2H), 2.63 (d, J=2.0 Hz, 2H), 2.44 (d, J=5.6 Hz, 2H), 2.28 (s, 1H), 2.05 (s, 1H), 1.23 (s, 1H), 1.03 (s, 2H), 1.01 (s, 3H), 0.99 (s, 2H), 0.92 (s, 3H), 0.11 (s, 2H). LCMS: (M+1: 563.3).

Step 4. To a solution of (10S,17E)-14-(2-hydroxyethyl)-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (140 mg, 249 mol, 1 eq) in DMF (2 mL) was added commercially available 2-iodoacetonitrile (62.3 mg, 373 mol, 1.5 eq) and K₂CO₃ (103 mg, 746 μmol, 3 eq). The mixture was stirred at 80° C. for 1 h. On completion, the reaction mixture was diluted with H₂O (10 mL) and extracted with EA (8 mL×3). The combined organic layers were washed with H₂O (15 mL), dried over Na₂SO₄, filtered and concentrated under reduced pressure to give {[(10S,17E)-14-(2-hydroxyethyl)-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (130 mg, crude) as an orange oil. LCMS: (M+1: 602.2).

Step 5. To a solution of {[(10S,17E)-14-(2-hydroxyethyl)-6,8,10,12,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (100 mg, 166 μmol, 1 eq) in DCM (1 mL) was added TFA (2.69 mmol, 0.2 mL, 16.2 eq). The mixture was stirred at 25° C. for 1 h. On completion, the reaction was concentrated in vacuum to give a black brown oil (70.0 mg, crude).

This brown oil (70.0 mg, 114 mol, 1 eq) was dissolved in MeOH (1 mL) and K₂CO₃(47.3 mg, 342 mol, 3 eq) was added. The mixture was stirred at 25° C. for 0.5 h. On completion, the reaction solution was filtered and then was concentrated in vacuum to give a residue. The residue was purified by prep-HPLC purification (column: Waters xbridge 150*25 mm 10 um; mobile phase: [water (NH4HCO₃)—ACN]; gradient: 23%-53% B over 10 min) to give Ex. 63 (12.93 mg, 23.16 μmol, 20.30% yield, 92.72% purity) as a yellow solid. LCMS: (M+1: 518.1). ¹H NMR for Ex. 63 is shown in the below NMR table.

### Preparation of {[(17E)-12-ethyl-4-[(2S)-1-hydroxypropan-2-yl]-6,8-dimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 66)

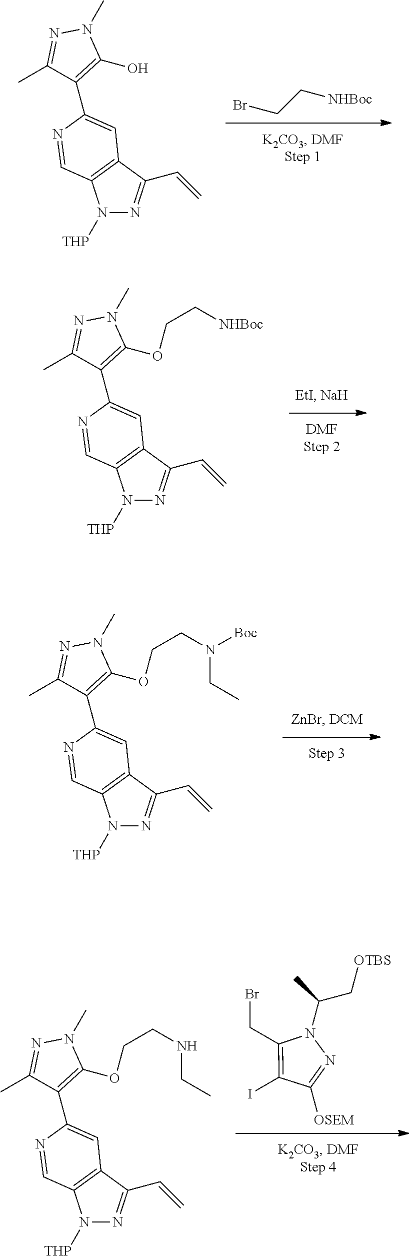

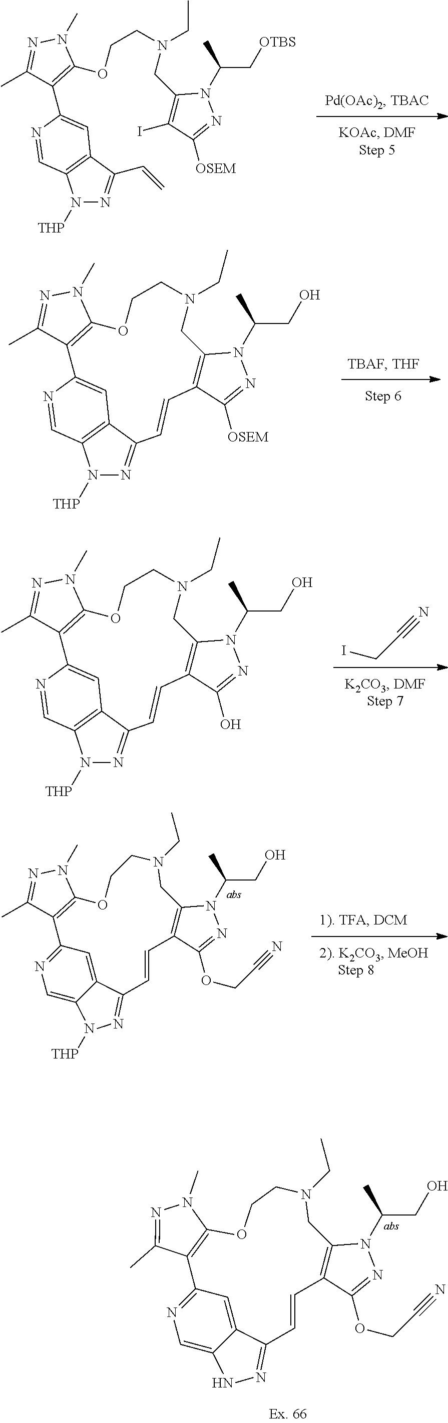

Step 1. To a solution of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (2.00 g, 5.89 mmol, 1 eq) in DMF (20 mL) was added commercially available tert-butyl N-(2-bromoethyl)carbamate (1.72 g, 7.66 mmol, 1.3 eq) and K₂CO₃ (2.44 g, 17.7 mmol, 3 eq). The mixture was stirred at 80° C. for 2 h. On completion, the reaction mixture was diluted with H₂O (200 mL) and extracted with EA (100 mL×3). The combined organic layers were washed with H₂O (300 mL), dried over Na₂SO₄, filtered and concentrated under reduced pressure to give a residue. The residue was purified by column chromatography (SiO₂, Petroleum ether/THF=1:0 to 1:1) to give tert-butyl N-[2-[2,5-dimethyl-4-(1-tetrahydropyran-2-yl-3-vinyl-pyrazolo[3,4-c]pyridin-5-yl)pyrazol-3-yl]oxyethyl]carbamate (1.83 g, 3.64 mmol, 62% yield, 96% purity) as an orange oil. ¹H NMR (400 MHz, DMSO-d₆) δ=9.24 (s, 1H), 7.94 (s, 1H), 7.12-6.99 (m, 2H), 6.14 (d, J=18.0 Hz, 1H), 5.98 (d, J=2.0, 9.5 Hz, 1H), 5.59 (d, J=11.8 Hz, 1H), 4.03 (q, J=7.2 Hz, 1H), 3.93 (t, J=5.2 Hz, 2H), 3.62 (s, 2H), 3.44 (s, 1H), 3.30 (s, 1H), 3.20 (d, J=5.6 Hz, 2H), 2.29-2.16 (m, 3H), 1.99 (s, 3H), 1.61 (d, J=3.2 Hz, 2H), 1.35 (d, J=3.6 Hz, 9H), 1.17 (t, J=7.2 Hz, 1H). LCMS: (M+1: 483.2).

Step 2. To a solution of tert-butyl N-[2-[2,5-dimethyl-4-(1-tetrahydropyran-2-yl-3-vinyl-pyrazolo[3,4-c]pyridin-5-yl)pyrazol-3-yl]oxyethyl]carbamate (1.70 g, 3.52 mmol, 1 eq) in DMF (17 mL) was added NaH (423 mg, 10.6 mmol, 60% purity, 3 eq) at 0° C. for 0.5 h, and then EtI (5.28 mmol, 423 μL, 1.5 eq) was added at 0° C. The mixture was stirred at 25° C. for 2 h. On completion, the reaction mixture was diluted with sat. NH₄Cl (100 mL) and extracted with EA (150 mL×3). The combined organic layers were washed with brine, dried over Na₂SO₄, filtered and concentrated under reduced pressure to give a residue. The residue was purified by column chromatography (SiO₂, Petroleum ether/Ethyl acetate=1:0 to 1:1) to give tert-butyl N-[2-[2,5-dimethyl-4-(1-tetrahydropyran-2-yl-3-vinyl-pyrazolo[3,4-c]pyridin-5-yl)pyrazol-3-yl]oxyethyl]-N-ethyl-carbamate (1.79 g, 3.19 mmol, 91% yield, 91% purity) as an orange oil. LCMS: (M+1: 511.6).

Step 3. To a solution of tert-butyl N-[2-[2,5-dimethyl-4-(1-tetrahydropyran-2-yl-3-vinyl-pyrazolo[3,4-c]pyridin-5-yl)pyrazol-3-yl]oxyethyl]-N-ethyl-carbamate (1.69 g, 3.31 mmol, 1 eq) in DCM (17 mL) was added ZnBr₂ (2.98 g, 13.2 mmol, 4 eq). The mixture was stirred at 25° C. for 16 h. On completion, the reaction mixture diluted with H₂O (50 mL) and extracted with DCM (25 mL×3). The combined organic layers were washed with brine, dried over Na₂SO₄, filtered and concentrated under reduced pressure to give a residue. The residue was purified by column chromatography (SiO₂, DCM/MeOH=1:0 to 10:1) to give 2-[2,5-dimethyl-4-(1-tetrahydropyran-2-yl-3-vinyl-pyrazolo[3,4-c]pyridin-5-yl)pyrazol-3-yl]oxy-N-ethyl-ethanamine (1.30 g, 2.91 mmol, 88% yield, 92% purity) as an orange oil. ¹H NMR (400 MHz, DMSO-d₆) δ=9.25 (d, J=1.2 Hz, 1H), 8.00-7.93 (m, 2H), 6.16 (d, J=18.0 Hz, 1H), 5.99 (d, J=1.6, 9.6 Hz, 1H), 5.75 (s, 1H), 5.59 (d, J=12.0 Hz, 1H), 4.08 (t, J=5.2 Hz, 2H), 3.67 (s, 3H), 3.51 (d, J=7.2, 8.8 Hz, 1H), 2.98 (t, J=5.2 Hz, 2H), 2.89 (s, 2H), 2.73 (s, 2H), 2.28 (s, 3H), 1.35 (s, 3H), 1.19-1.16 (m, 2H), 1.06-1.02 (m, 3H). LCMS: (M+1: 411.2).

Step 4. To a solution of 2-[2,5-dimethyl-4-(1-tetrahydropyran-2-yl-3-vinyl-pyrazolo[3,4-c]pyridin-5-yl)pyrazol-3-yl]oxy-N-ethyl-ethanamine (1.20 g, 2.92 mmol, 1 eq) in DMF (12 mL) was added 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, (1.77 g, 2.92 mmol, 1 eq) and K₂CO₃ (1.01 g, 7.31 mmol, 2.5 eq). The mixture was stirred at 80° C. for 2 h. On completion, the reaction mixture diluted with H₂O (60 mL) and extracted with EA (50 mL×3). The combined organic phase was dried over anhydrous sodium sulfate, filtered and concentrated under reduced pressure to give a residue. The residue was purified by column chromatography (SiO₂, Petroleum ether/THF=1:0 to 3:1) to give N-[[2-[(1S)-2-[tert-butyl(dimethyl)silyl]oxy-1-methyl-ethyl]-4-iodo-5-(2-trimethylsilylethoxymethoxy)pyrazol-3-yl|methyl|-2-[2,5-dimethyl-4-(1-tetrahydropyran-2-yl-3-vinyl-pyrazolo[3,4-c]pyridin-5-yl)pyrazol-3-yl]oxy-N-ethyl-ethanamine (1.54 g, 1.42 mmol, 48% yield, 86% purity) as a colorless oil. LCMS: (M+1: 935.2).

Step 5. To a solution of N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-ethanamine (1.30 g, 1.39 mmol, 1 eq) in DMF (13 mL) was added Pd(OAc)₂ (46.8 mg, 209 μmol, 0.15 eq), TBAC (773 mg, 2.78 mmol, 2 eq) and KOAc (682 mg, 6.95 mmol, 5 eq). The mixture was stirred at 80° C. for 1 h under N₂ atmosphere. On completion, the reaction mixture diluted with H₂O (100 mL) and extracted with EA (50 mL×3). The combined organic layers were washed with H₂O (200 mL), dried over Na₂SO₄, filtered and concentrated under reduced pressure to give a residue. The residue was purified by column chromatography (SiO₂, Petroleum ether/THF=1:0 to 1:1) to give (2S)-2-[(17E)-12-ethyl-6,8-dimethyl-2-(oxan-2-yl)-16-{[2-(trimethylsilyl)ethoxy]methoxy}-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (537 mg, 418 mol, 30% yield, 54% purity) as an orange oil. LCMS: (M+1: 693.3).

Step 6. To a solution of (2S)-2-[(17E)-12-ethyl-6,8-dimethyl-2-(oxan-2-yl)-16-{[2-(trimethylsilyl)ethoxy]methoxy}-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (250 mg, 361 mol, 1 eq) in THE (1 mL) was added TBAF (1 M, 4 mL, 11.1 eq). The mixture was stirred at 70° C. for 48 h. On completion, the reaction mixture was diluted with H₂O (10 mL) and extracted with EA (12 mL×3). The combined organic phase was dried over anhydrous sodium sulfate, filtered and concentrated under reduced pressure to give a residue. The residue was purified by column chromatography (SiO₂, PE/THF=1:0 to 1:2) to give (17E)-12-ethyl-14-[(2S)-1-hydroxypropan-2-yl]-6,8-dimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (150 mg, 187 μmol, 52% yield, 70% purity) as an orange solid. ¹H NMR (400 MHz, DMSO-d₆) δ=10.56 (s, 1H), 9.18-9.12 (m, 1H), 8.52 (s, 1H), 7.98 (d, J=17.2 Hz, 1H), 7.16 (d, J=16.8 Hz, 1H), 6.87 (s, 6H), 6.65 (s, 3H), 5.89 (d, J=10.4 Hz, 1H), 4.88-4.81 (m, 1H), 3.68 (s, 2H), 2.33 (s, 1H), 2.18 (s, 8H), 1.99 (s, 1H), 1.31-1.27 (m, 4H), 1.17 (t, J=7.2 Hz, 2H), 0.93 (t, J=7.2 Hz, 4H). LCMS: (M+1: 563.2).

Step 7. To a solution of (17E)-12-ethyl-14-[(2S)-1-hydroxypropan-2-yl]-6,8-dimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (130 mg, 231 μmol, 1 eq) in DMF (1.5 mL) was added commercially available 2-iodoacetonitrile (57.9 mg, 346 μmol, 1.5 eq) and K₂CO₃ (95.8 mg, 693 μmol, 3 eq). The mixture was stirred at 80° C. for 1 h. On completion, the reaction mixture was diluted with H₂O (10 mL) and extracted with EA (12 mL×3). The combined organic layers were washed with H₂O (20 mL), dried over Na₂SO₄, filtered and concentrated under reduced pressure to give {[(17E)-12-ethyl-14-[(2S)-1-hydroxypropan-2-yl]-6,8-dimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (80.0 mg, crude) as an orange solid. LCMS: (M+1: 602.1).

Step 8. To a solution of {[(17E)-12-ethyl-14-[(2S)-1-hydroxypropan-2-yl]-6,8-dimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (60.0 mg, 99.7 μmol, 1 eq) in DCM (0.6 mL) was added TFA (1.62 mmol, 120 μL, 16.2 eq). The mixture was stirred at 25° C. for 2 h. On completion, the reaction was concentrated in vacuum to give a black brown oil (50.0 mg, crude). LCMS: (M+1: 614.1).

This black, brown oil (40.0 mg, 65.2 mol, 1 eq) was dissolved in MeOH (0.4 mL) and K₂CO₃ (27.0 mg, 196 μmol, 3 eq) was added. The mixture was stirred at 25° C. for 0.5 hour. The reaction solution was filtered and concentrated in vacuum to give a residue. The filtrate was purified by prep-HPLC purification (column: Welch Ultimate Cis 150*25 mm*5 um; mobile phase: [water (FA)-ACN]; gradient: 12%-42% B over 10 min) to give Ex. 66 (16.34 mg, 31.18 mol, 47.82% yield, 98.75% purity) as a yellow solid. LCMS: (M+1: 518.2). ¹H NMR for Ex. 66 is shown in the below NMR table.

### Preparation of {[(17E)-14-[(2S)-1-hydroxypropan-2-yl]-6,8,12-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 70)

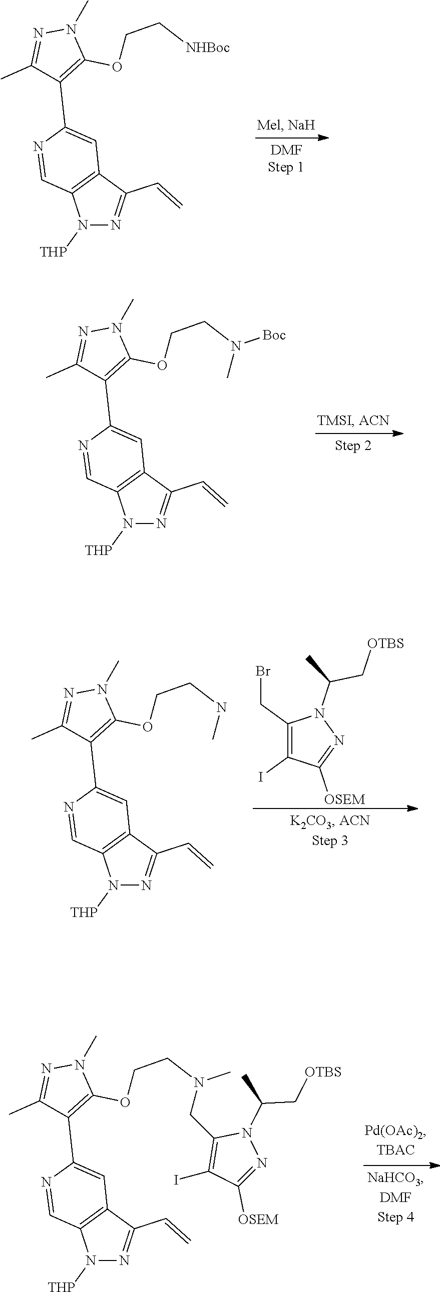

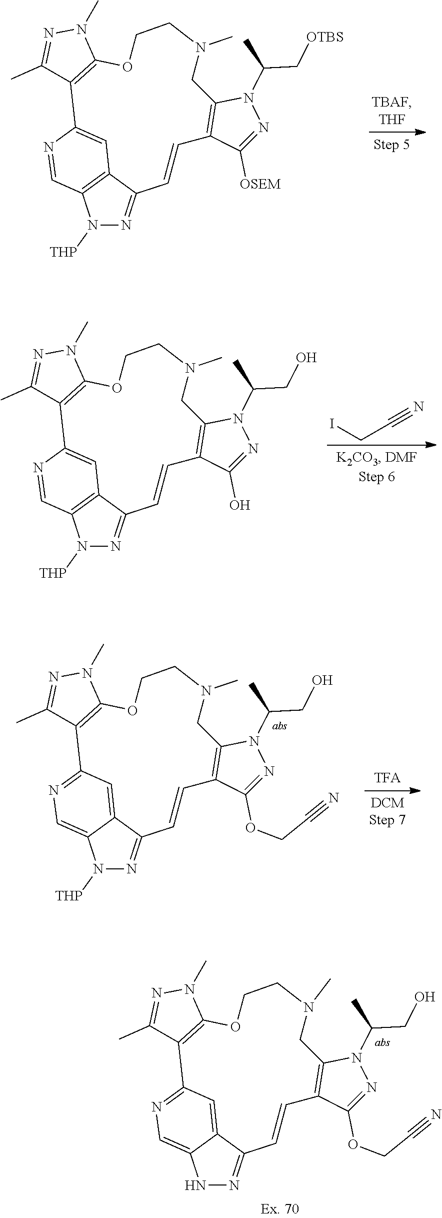

Step 1. A mixture of tert-butyl N-[2-[2,5-dimethyl-4-(1-tetrahydropyran-2-yl-3-vinyl-pyrazolo[3,4-c]pyridin-5-yl)pyrazol-3-yl]oxyethyl]carbamate, which was prepared according to the method described in Ex. 66, (2.00 g, 4.14 mmol, 1 eq) in DMF (25 mL) was added NaH (249 mg, 6.22 mmol, 60% purity, 1.5 eq) at 0° C. for 0.5 h, and then add MeI (824 mg, 5.80 mmol, 1.4 eq). The mixture was stirred at 25° C. for 2 h. On completion, the reaction mixture was quenched by addition sat. NH₄Cl (10 mL) at 0° C., and then diluted with H₂O (80 mL) and extracted with EA (30 mL×3). The combined organic layers were washed with H₂O (30 mL×3), dried over Na₂SO₄, filtered, and concentrated under reduced pressure to give a residue. The residue was purified by column chromatography on silica gel (12 g silica gel, EA in Petroleum ether from 0% to 100%) to give tert-butyl N-[2-[2,5-dimethyl-4-(1-tetrahydropyran-2-yl-3-vinyl-pyrazolo[3,4-c]pyridin-5-yl)pyrazol-3-yl]oxyethyl]-N-methyl-carbamate (2.20 g, 4.08 mmol, 98% yield, 92% purity) as a brown oil. LCMS: (M+1: 497.2).

Step 2. To a solution of tert-butyl N-[2-[2,5-dimethyl-4-(1-tetrahydropyran-2-yl-3-vinyl-pyrazolo[3,4-c]pyridin-5-yl)pyrazol-3-yl]oxyethyl]-N-methyl-carbamate (2.10 g, 4.23 mmol, 1 eq) in ACN (20 mL) was added TMSI (1.10 g, 5.50 mmol, 1.3 eq) at 0° C. The mixture was stirred at 0° C. for 2 h. On completion, the reaction mixture was quenched by addition sat. NaHCO₃ (10 mL) at 0° C., and then diluted with H₂O (40 mL) and extracted with EA (20 mL×3). The combined organic layers were washed with H₂O (20 mL×3), dried over Na₂SO₄, filtered and concentrated under reduced pressure to give a residue. The residue was purified by column chromatography on silica gel (4 g silica gel, EA in Petroleum ether from 0% to 100%) to give 2-[2,5-dimethyl-4-(1-tetrahydropyran-2-yl-3-vinyl-pyrazolo[3,4-c]pyridin-5-yl)pyrazol-3-yl]oxy-N-methyl-ethanamine (1.10 g, 2.77 mmol, 65% yield, 100% purity) as a brown oil. LCMS: (M+1: 397.1).

Step 3. A solution of 2-[2,5-dimethyl-4-(1-tetrahydropyran-2-yl-3-vinyl-pyrazolo[3,4-c]pyridin-5-yl)pyrazol-3-yl]oxy-N-methyl-ethanamine (1.00 g, 2.52 mmol, 1 eq), 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, (2.46 g, 2.52 mmol, 1 eq) and K₂CO₃ (1.05 g, 7.57 mmol, 3 eq) in ACN (20 mL) was stirred at 60° C. for 2 h. On completion, the reaction mixture was concentrated in vacuo. The residue was purified by column chromatography on silica gel (12 g silica gel, EA in Petroleum ether from 0% to 100%) to give N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-ethanamine (2.10 g, 1.48 mmol, 58% yield, 65% purity) as a brown oil. LCMS: (M+1: 921.1).

Step 4. To a solution of N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-ethanamine (2.00 g, 2.17 mmol, 1 eq), NaHCO₃ (456 mg, 5.43 mmol, 2.5 eq), TBAC (603 mg, 2.17 mmol, 1 eq), diacetoxypalladium (97.5 mg, 434 μmol, 0.2 eq) in DMF (200 mL) was degassed and purged with N₂ for 3 times, and then the mixture was stirred at 130° C. for 2 h under N₂ atmosphere. On completion, the mixture was poured into H₂O (500 mL) and extracted with EA (100 mL×3). The combined organic phase was washed with H₂O (100 mL×3) and dried over anhydrous sodium sulfate, filtered and concentrated to give a residue. The residue was purified by column chromatography (SiO₂, DCM/THF=3:1 to 0:1) to give (17E)-14-[(2S)-1-{[tert-butyl(dimethyl)silyl]oxy}propan-2-yl]-6,8,12-trimethyl-2-(oxan-2-yl)-16-{1[2-(trimethylsilyl)ethoxy]methoxy}-2,10,11,12,13,14-hexahydro-8H-5,3-(azenometheno)tripyrazolo[3,4-f3′,4′-j:4″,3″-n][1,4]oxazacyclopentadecine (1.05 g, 922 μmol, 42% yield, 70% purity) as a brown solid. LCMS: (M+1: 793.2).

Step 5. A solution of (17E)-14-[(2S)-1-{[tert-butyl(dimethyl)silyl]oxy}propan-2-yl]-6,8,12-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f3′,4′-j:4″,3″-n][1,4]oxazacyclopentadecine (1.00 g, 1.26 mmol, 1 eq) in TBAF (1 M in THF, 7.56 mL, 6 eq) was stirred at 70° C. for 16 h. On completion, the mixture was diluted with water (30 mL) and extracted with ethyl acetate (15 mL×3). The combined organic phase was washed with saturated sodium chloride solution (30 mL×2) and dried over Na₂SO₄, filtered and the filtrate was concentrated to give a residue. The residue was purified by column chromatography on silica gel (4 g silica gel, MeOH in DCM ether from 0% to 10%) to give (17E)-14-[(2S)-1-hydroxypropan-2-yl]-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (365 mg, 550 mol, 44% yield, 83% purity) as a brown solid. LCMS: (M+1: 549.1).

Step 6. To a mixture of (17E)-14-[(2S)-1-hydroxypropan-2-yl]-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (340 mg, 620 mol, 1 eq) and K₂CO₃ (257 mg, 1.86 mmol, 3 eq) in DMF (4 mL) was added commercially available 2-iodoacetonitrile (103 mg, 620 mol, 1 eq). The mixture was stirred at 25° C. for 1 h. On completion, the mixture was diluted with water (30 mL) and extracted with ethyl acetate (15 mL×3). The combined organic phase was washed with saturated sodium chloride solution (5 mL×3) and dried over Na₂SO₄, filtered and the filtrate was concentrated to give {[(17E)-14-[(2S)-1-hydroxypropan-2-yl]-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (386 mg, 574 mol, 92% yield, 87% purity) as a brown solid. LCMS: (M+1: 588.2).

Step 7. To a solution of {[(17E)-14-[(2S)-1-hydroxypropan-2-yl]-6,8,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (300 mg, 510 μmol, 1 eq) in DCM (3 mL) was added TFA (20.2 mmol, 1.50 mL, 39 eq). The mixture was stirred at 25° C. for 1 h. On completion, the reaction mixture was concentrated in vacuo. The mixture was purified by Prep-HPLC (column: Welch Ultimate C18 150*25 mm*5 um; mobile phase: [water(FA)-ACN]; gradient:6%-36% B over 10 min). Then the solvent was lyophilized to give Ex. 70 (40 mg, 66.96 mol, 13% yield, 92% purity, FA) as a yellow solid. LCMS: (M+1: 504.2). ¹H NMR for Ex. 70 is shown in the below NMR table.

### Preparation of (2S)-1-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol (Ex. 86)

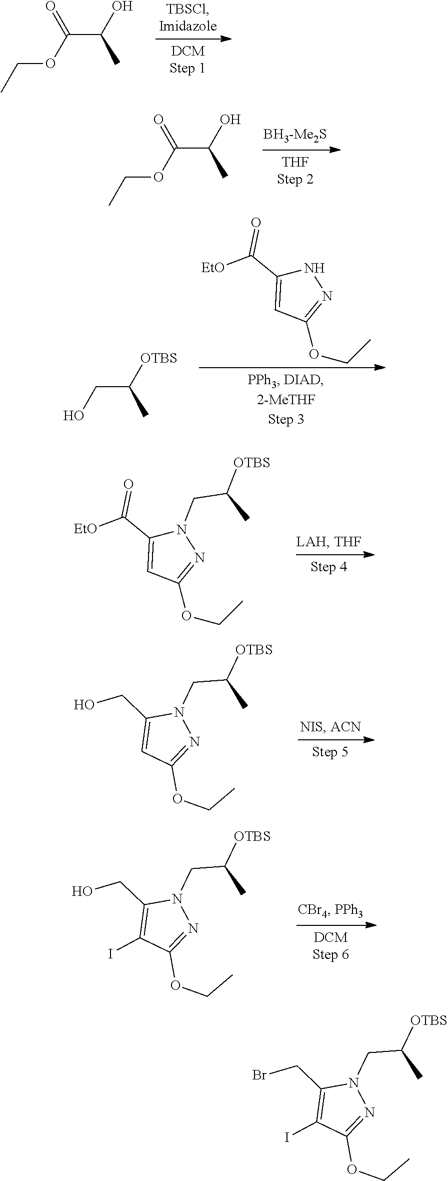

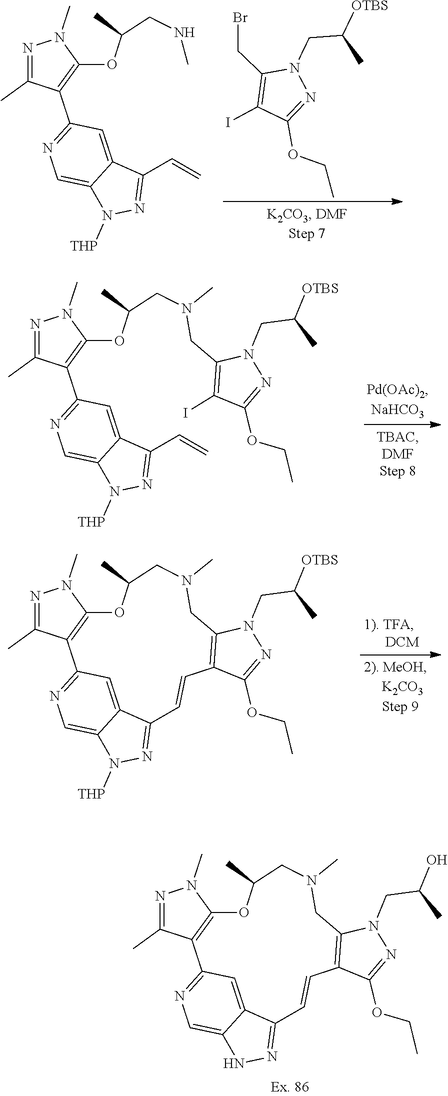

Step 1. To a solution of commercially available ethyl (2S)-2-hydroxypropanoate (3.00 g, 25.4 mmol, 1 eq) in DCM (30 mL) was added imidazole (5.19 g, 76.2 mmol, 3 eq), and TBSCl (5.74 g, 38.1 mmol, 1.5 eq) was then added at 0° C. The mixture was stirred at 25° C. for 1 h. On completion, the mixture was quenched with dilute citric acid (100 mL) and extracted with DCM (50 mL×3). The combined organic phase was dried over anhydrous Na₂SO₄, filtered and the filtrate was concentrated to give a residue. The residue was purified by column chromatography (SiO₂, PE:EA=10:1) to give ethyl (2S)-2-[tert-butyl(dimethyl)silyl]oxypropanoate (5.80 g, crude) as a white oil. ¹H NMR (400 MHz, DMSO-d₆) δ=4.32 (d, J=6.8 Hz, 1H), 4.15-4.03 (m, 2H), 1.30-1.26 (m, 3H), 1.21-1.16 (m, 3H), 0.86 (s, 9H), 0.04 (d, J=3.4 Hz, 6H).

Step 2. To a solution of ethyl (2S)-2-[tert-butyl(dimethyl)silyl]oxypropanoate (3.00 g, 12.9 mmol, 1 eq) in THE (30 mL), N₂ replacement was carried out and BH₃-Me₂S (10 M, 2.58 mL, 2 eq) was then added at 0° C. The mixture was stirred at 25° C. for 0.5 h. Then, the mixture was stirred at 70° C. for 2 h. After N₂ replacement, the mixture was stirred at 130° C. for 1 h. On completion, the mixture was diluted with MeOH (10 mL) and concentrated to give a residue. The residue was purified by column chromatography (SiO₂, PE:EA=10:1) to give (2S)-2-[tert-butyl(dimethyl)silyl]oxypropan-1-ol (1.96 g, crude) as a yellow oil. ¹H NMR (400 MHz, DMSO-d₆) δ=4.54 (t, J=5.7 Hz, 1H), 3.77-3.68 (m, 1H), 3.31-3.10 (m, 2H), 1.04 (d, J=6.1 Hz, 3H), 0.85 (s, 9H), 0.03 (s, 6H).

Step 3. To a solution of (2S)-2-[tert-butyl(dimethyl)silyl]oxypropan-1-ol (1.81 g, 9.53 mmol, 1.5 eq), commercially available ethyl 3-ethoxy-1H-pyrazole-5-carboxylate (1.17 g, 6.35 mmol, 1 eq) in 2-MeTHF (20 mL) was added PPh₃ (3.67 g, 14.0 mmol, 2.2 eq). The mixture was stirred at 25° C. for 0.5 h, and then DIAD (2.83 g, 14.0 mmol, 2.2 eq) was added at 0° C. The mixture was stirred at 25° C. for 2 h. The residue was purified by column chromatography (SiO₂, PE:EA=10:1) to give ethyl 2-[(2S)-2-[tert-butyl(dimethyl)silyl]oxypropyl]-5-ethoxy-pyrazole-3-carboxylate (1.96 g, crude) as a yellow oil. LCMS: (M+1:357.2).

Step 4. To a solution of ethyl 2-1(2S)-2-[tert-butyl(dimethyl)silyl|oxypropyl|-5-ethoxy-pyrazole-3-carboxylate (1.76 g, 4.94 mmol, 1 eq) in THE (18 mL) was added LAH (2.5 M, 2.96 mL, 1.5 eq) at 0° C. The mixture was stirred at 25° C. for 1 h. On completion, the mixture was diluted with water (0.3 mL), 15% NaOH (0.3 ml) and water (0.9 mL). The combined organic phase was dried over Na₂SO₄, filtered and the filtrate was concentrated. The residue was purified by column chromatography (SiO₂, PE:THF=4:1) to give [2-[(2S)-2-[tert-butyl(dimethyl)silyl]oxypropyl]-5-ethoxy-pyrazol-3-yl]methanol (1.46 g, crude) as a yellow oil. LCMS: (M+1:315.3).

Step 5. To a solution of [2-[(2S)-2-[tert-butyl(dimethyl)silyl]oxypropyl]-5-ethoxy-pyrazol-3-yl]methanol (1.26 g, 4.01 mmol, 1 eq) in ACN (13 mL) was added NIS (992 mg, 4.41 mmol, 1.1 eq) at 0° C. The mixture was stirred at 25° C. for 1 h. On completion, the mixture was quenched with saturated Na₂SO₃(20 mL) at 0° C. and extracted with ethyl acetate (15 mL×3). The combined organic phase was dried over anhydrous Na₂SO₄, filtered and the filtrate was concentrated, and the residue was purified by column chromatography (SiO₂, PE: THF=10:1) to give [2-[(2S)-2-[tert-butyl(dimethyl)silyl]oxypropyl]-5-ethoxy-4-iodo-pyrazol-3-yl]methanol (2.05 g, crude) as a yellow oil. LCMS: (M+1:441.1).

Step 6. To a solution of [2-[(2S)-2-[tert-butyl(dimethyl)silyl]oxypropyl]-5-ethoxy-4-iodo-pyrazol-3-yl]methanol (1.85 g, 4.20 mmol, 1 eq) in DCM (20 mL) was added PPh₃ (1.32 g, 5.04 mmol, 1.2 eq). After N₂ replacement, CBr₄ (1.67 g, 5.04 mmol, 1.2 eq) in DCM (5 mL) was added at 0° C. The mixture was stirred at 25° C. for 1 h. On completion, the mixture was concentrated, and the residue was purified by column chromatography (SiO₂, PE:THF=10:1) to give [(1S)-2-[5-(bromomethyl)-3-ethoxy-4-iodo-pyrazol-1-yl]-1-methyl-ethoxy]-tert-butyl-dimethyl-silane (1.45 g, crude) as a yellow oil. LCMS: (M+1:503.0).

Step 7, Step 8, and Step 9 were carried out in the same manner as in Ex. 27, using (2S)-2-[2,5-dimethyl-4-(1-tetrahydropyran-2-yl-3-vinyl-pyrazolo[3,4-c]260yridine-5-yl)pyrazol-3-yl]oxy-N-methyl-propan-1-amine that was prepared according to the method described in Ex. 22. ¹H NMR for Ex. 86 can be found in the below NMR table.

### Preparation of (2S)-2-[(10S,17E)-16-ethoxy-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 91)

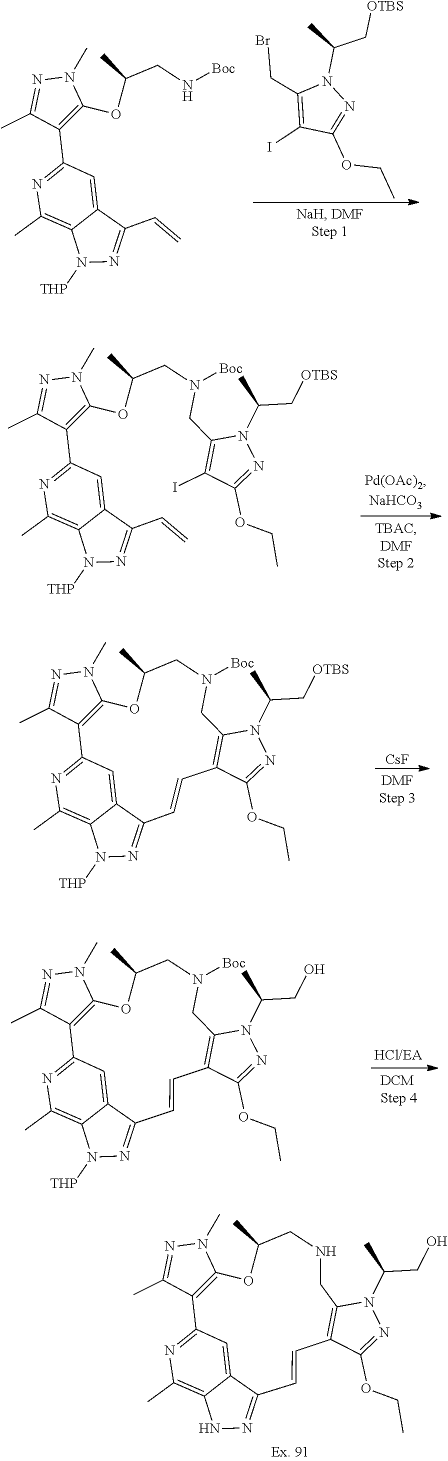

Step 1. To a solution of tert-butyl N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]carbamate, which was prepared according to the method described in Ex. 47, (1.00 g, 1.96 mmol, 1 eq) in DMF (10 mL) was added NaH (235 mg, 5.88 mmol, 60% purity, 3 eq) at 0° C. and stirred for 0.5 hour, followed by addition of [(2S)-2-[5-(bromomethyl)-3-ethoxy-4-iodo-pyrazol-1-yl]propoxy]-tert-butyl-dimethyl-silane, which was prepared according to the method described in Ex. 19, (1.58 g, 3.13 mmol, 1.6 eq). The mixture was stirred at 25° C. for 2 hours. On completion, the reaction mixture diluted with sat. NH₄Cl (200 mL) and extracted with EA (100 mL×3). The combined organic phase was dried over anhydrous sodium sulfate, filtered and concentrated under reduced pressure to give tert-butyl N-[[2-[(1S)-2-[tert-butyl(dimethyl)silyl]oxy-1-methyl-ethyl]-5-ethoxy-4-iodo-pyrazol-3-yl]methyl]-N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]carbamate (1.60 g, 1.30 mmol, 67% yield, 76% purity) as an orange oil. LCMS: (M+1: 933.5).

Step 2 and Step 3. To a solution of tert-butyl N-[[2-[(1S)-2-[tert-butyl(dimethyl)silyl]oxy-1-methyl-ethyl]-5-ethoxy-4-iodo-pyrazol-3-yl]methyl]-N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]carbamate (1.50 g, 1.61 mmol, 1 eq) in DMF (15 mL) was added NaHCO₃ (675 mg, 8.04 mmol, 5 eq), TBAC (894 mg, 3.22 mmol, 2 eq) and Pd(OAc)₂ (54.1 mg, 241 μmol, 0.15 eq). The mixture was stirred at 130° C. for 1 hour under N₂ atmosphere. Then CsF (1.47 g, 9.65 mmol, 6 eq) was added and the mixture was stirred at 25° C. for 16 hours. On completion, the reaction mixture was diluted with H₂O (200 mL) and extracted with EA (150 mL×3). The combined organic phase was dried over anhydrous sodium sulfate, filtered and concentrated under reduced pressure to give tert-butyl (10S,17E)-16-ethoxy-14-[(2S)-1-hydroxypropan-2-yl]-6,8,10,20-tetramethyl-2-(oxan-2-yl)-10,11,13,14-tetrahydro-8H-5,3-(azenometheno)tripyrazolo[3,4-f:3′,4′-j:4″,3″-n][1,4]oxazacyclopentadecine-12(2H)-carboxylate (600 mg, crude) as a black brown oil. ¹H NMR (400 MHz, DMSO-d₆) δ=7.95 (s, 1H), 7.66-7.53 (m, 2H), 5.26 (s, 1H), 4.08-3.98 (m, 3H), 3.61 (d, J=1.6 Hz, 2H), 3.20-3.11 (m, 2H), 2.89 (s, 4H), 2.76-2.72 (m, 6H), 2.58 (s, 2H), 1.99 (s, 2H), 1.50 (s, 5H), 1.44 (t, J=2.4, 7.2 Hz, 3H), 0.97-0.90 (m, 3H), 0.88-0.80 (m, 9H), 0.01 (s, 1H), −0.04 (s, 4H). LCMS: (M+1: 691.3).

Step 4. To a solution of intermediate from Step 3 (300 mg, 434 mol, 1 eq) in DCM (3 mL) was added HCl/EtOAc (4.00 M, 3 mL, 27.6 eq). The mixture was stirred at 25° C. for 2 hours. On completion, the reaction was concentrated in vacuum, and the residue was purified by prep-HPLC purification to give Ex. 91 (43.83 mg, 81.75 μmol, 18.83% yield, 94.49% purity) as a yellow solid. LCMS: (M+1: 507.1). ¹H NMR for Ex. 91 is shown in the below NMR table.

### Preparation of (2S)-2-[(10S,17E)-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 92)

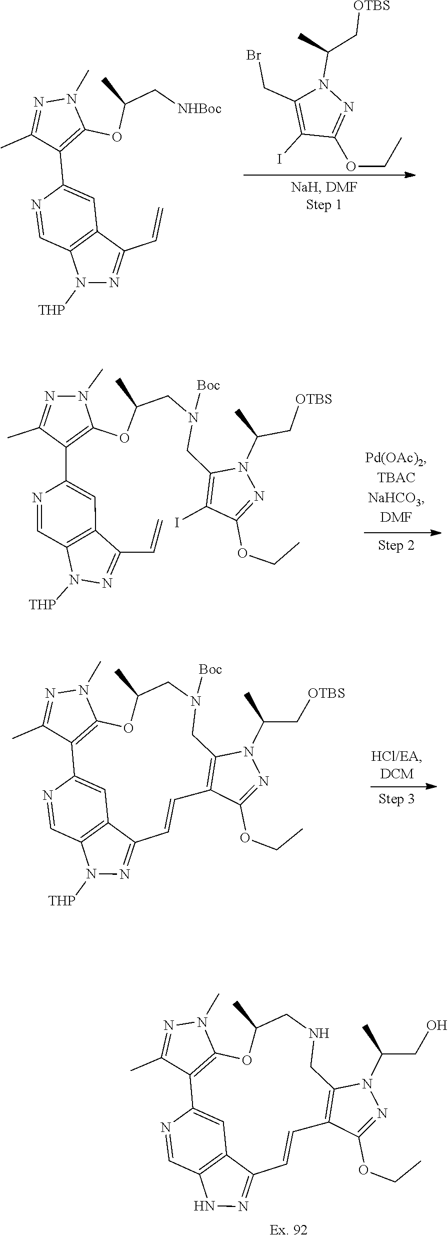

(2S)-2-[(10S,17E)-16-ethoxy-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 92) was prepared according to the method described in Ex. 91 using tert-butyl N-[(2S)-2-[2,5-dimethyl-4-(1-tetrahydropyran-2-yl-3-vinyl-pyrazolo[3,4-c]pyridin-5-yl)pyrazol-3-yl]oxypropyl]carbamate, which was prepared according to the method described in Ex. 22, and [(2S)-2-[5-(bromomethyl)-3-ethoxy-4-iodo-pyrazol-1-yl]propoxy]-tert-butyl-dimethyl-silane, which was prepared according to the method described in Ex. 19. ^{J}H NMR for Ex. 92 can be found in the below NMR table.

### Preparation of (2S)-2-[(11R,17E)-16-ethoxy-6,8,11,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 97)

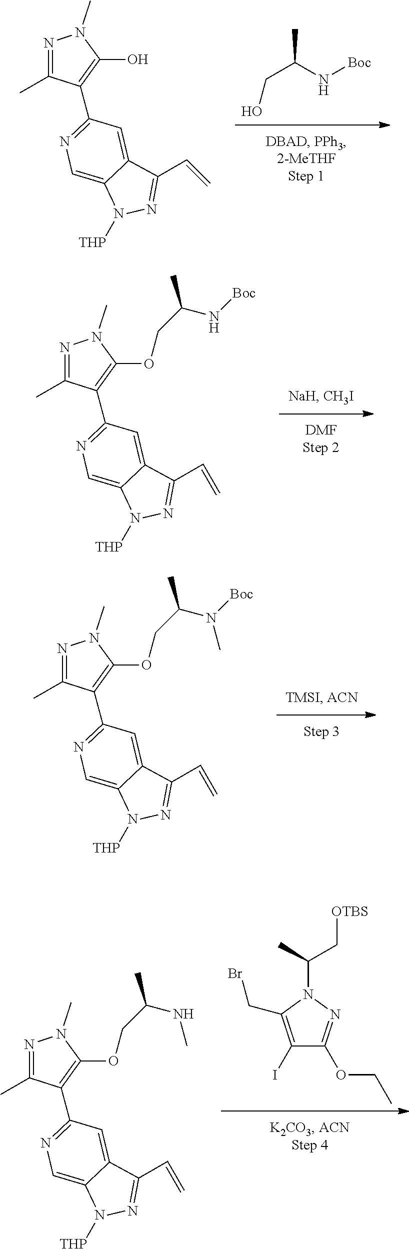

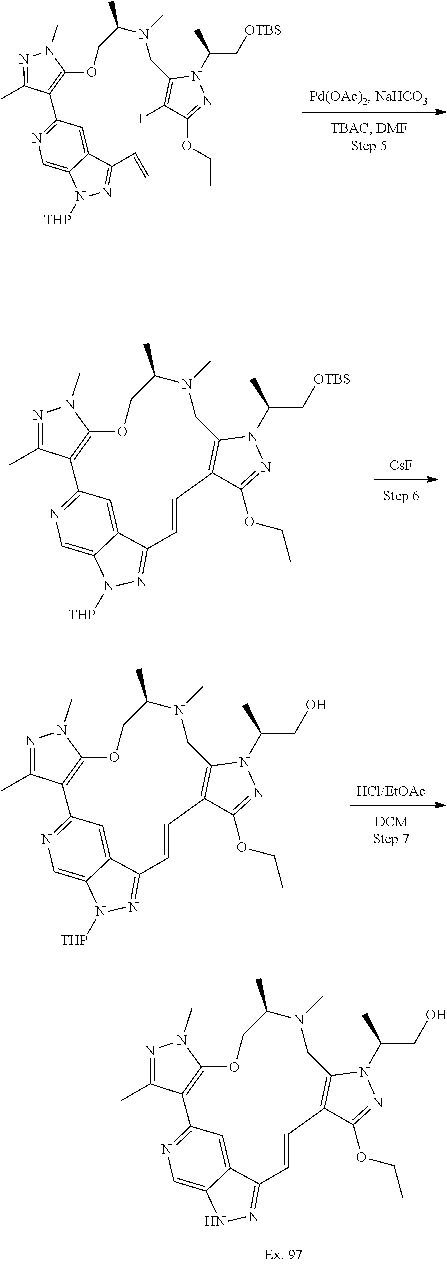

Steps 1 and 2 were performed in the same manner as steps 1 and 2 in Ex. 29, substituting 2-MeTHF for the solvent in step 1 and using 2,5-dimethyl-4-(1-tetrahydropyran-2-yl-3-vinyl-pyrazolo[3,4-c]pyridin-5-yl)pyrazol-3-ol, which was prepared according to the method described in Ex. 1 and commercially available tert-butyl N-[(1R)-2-hydroxy-1-methyl-ethyl]carbamate in Step 1.

Step 3. To a solution of tert-butyl ((2R)-1-((1,3-dimethyl-4-(1-(tetrahydro-2H-pyran-2-yl)-3-vinyl-1H-pyrazolo[3,4-c]pyridin-5-yl)-1H-pyrazol-5-yl)oxy)propan-2-yl)(methyl)carbamate (3.20 g, 2.26 mmol, 1 eq) in ACN (32 mL) was added TMSI (587 mg, 2.93 mmol, 1.3 eq) dropwise at 0° C. The mixture was stirred at 0° C. for 30 min. On completion, the mixture was concentrated in vacuum to give a residue. The residue was purified by column chromatography (SiO₂, Dichloromethane/Methanol=1:0 to 0:1) to give (2R)-1-((1,3-dimethyl-4-(1-(tetrahydro-2H-pyran-2-yl)-3-vinyl-1H-pyrazolo[3,4-c]pyridin-5-yl)-1H-pyrazol-5-yl)oxy)-N-methylpropan-2-amine (350 mg, 0.699 mmol, 31% yield, 82% purity) as a colorless oil.

Step 4 was performed in a similar manner to step 4 in Ex. 29.

Steps 5 through 7 were performed in a similar manner to steps 2 through 4 in Ex. 91. ¹H NMR for Ex. 97 can be found in the below NMR table.

### Preparation of 2-[(17E)-16-ethoxy-6,12-dimethyl-2,6,10,11,12,13-hexahydro-14H-5,3-(azenometheno)tripyrazolo[3,4-f:3′,4′-j:3″,4″-n][1,4]oxazacyclopentadecin-14-yl]ethan-1-ol (Ex. 104)

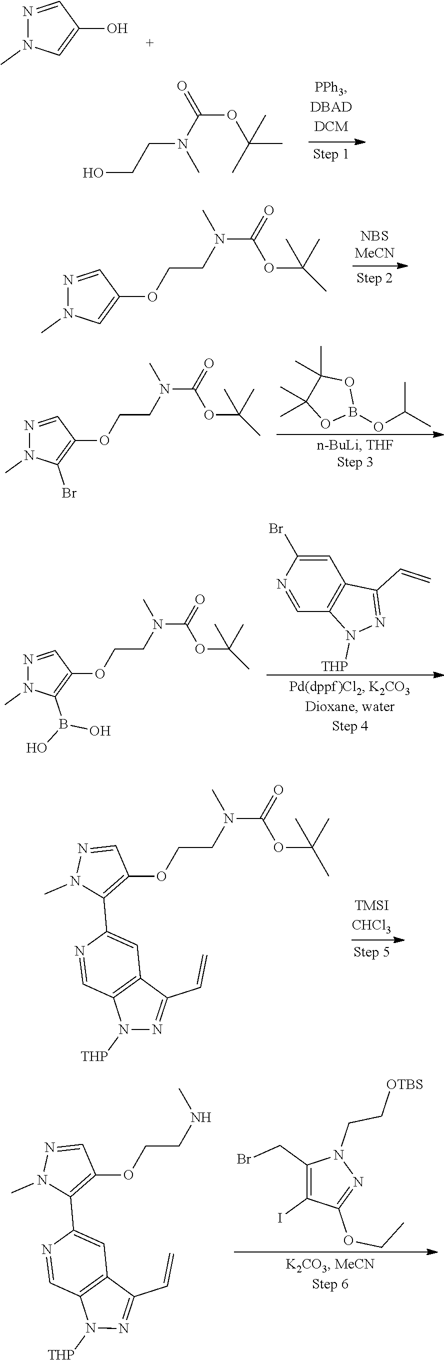

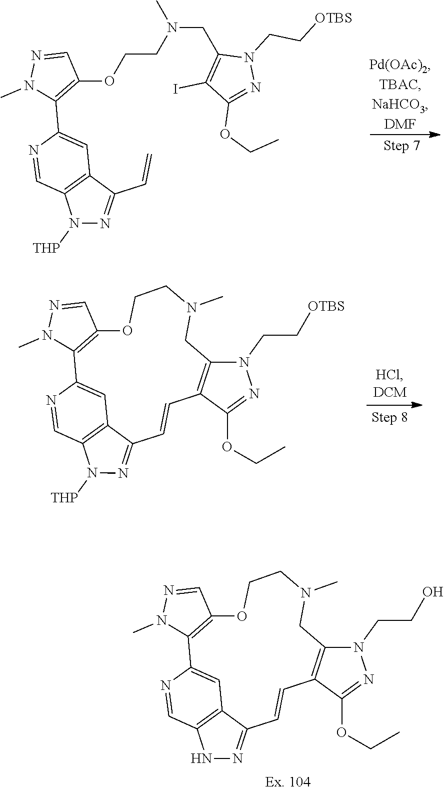

Step 1. To commercially available 1-methylpyrazol-4-ol (385 mg, 3.92 mmol), commercially available tert-butyl N-(2-hydroxyethyl)-N-methyl-carbamate (1.03 g, 5.89 mmol), and triphenylphosphine (1.54 g, 5.89 mmol) in dry DCM (7.85 mL) at 0° C. was added DBAD (1.45 g, 6.28 mmol). The mixture was stirred for 18 hr as temperature increased from 0° C. to room temperature. Flash column chromatography (automated system, 24 g silica, 0-50% EA in Hexanes) 2 times provided tert-butyl N-methyl-N-[2-(1-methylpyrazol-4-yl)oxyethyl]carbamate (1.48 g, 5.80 mmol, 148% yield) as a thick nearly colorless oil. It was used in the next step without further purification. LCMS: 256.3 (M+1)

Step 2. To tert-butyl N-methyl-N-[2-(1-methylpyrazol-4-yl)oxyethyl]carbamate (1 g, 3.92 mmol) in Acetonitrile (20 mL) was added NBS (627.4 mg, 3.53 mmol). The mixture was stirred at 22° C. for 3 days. The reaction was quenched with water (5 mL) and the volume decreased by ~1/3 under reduced pressure. The remaining solution was worked up with water and DCM (10 mL each). The layers were partitioned, and the aqueous layer extracted again with DCM (2×5 mL). The combined organic layer was washed with brine and dried over sodium sulfate. Flash column chromatography (automated system, 24 g silica, 0-50% EA in Hexanes) provided tert-butyl N-[2-(5-bromo-1-methyl-pyrazol-4-yl)oxyethyl]-N-methyl-carbamate (844 mg, 2.53 mmol, 64% yield) as an orange oil. LCMS: 278.0/280.0 (M-56)

Step 3. tert-butyl N-[2-(5-bromo-1-methyl-pyrazol-4-yl)oxyethyl]-N-methyl-carbamate (122 mg, 0.365 mmol) was dissolved in anhydrous THE (1 mL) and cooled to −78° C. under argon. To this solution was added n-BuLi (70.2 mg, 1.10 mmol, 0.438 mL of 2.5 M in hexane) dropwise. After stirring at −78° C. for 1 h, commercially available 2-isopropoxy-4,4,5,5-tetramethyl-1,3,2-dioxaborolane (135.84 mg, 0.73 mmol) was added under argon. After 30 minutes, the reaction mixture was removed from −78° C. and quenched with water (1 mL) and stirred vigorously. Volatiles were removed under reduced pressure and the crude product was used directly to the next step without further purification.

Step 4. To the mixture of [4-[2-[tert-butoxycarbonyl(methyl)amino]ethoxy]-2-methyl-pyrazol-3-yl]boronic acid (109 mg, 0.364 mmol) in dioxane (0.5 mL) and water (0.5 mL), was added 5-bromo-1-tetrahydropyran-2-yl-3-vinyl-pyrazolo[3,4-c]pyridine, which was prepared according to the method described in Ex. 1, (89.8 mg, 0.292 mmol) and K₂CO₃ (151.09 mg, 1.09 mmol). The reaction mixture was stirred as argon was bubbled through and catalyst, Pd(dppf)Cl₂ (26.7 mg, 0.036 mmol) was added. The vessel was sealed and heated to 80° C. for 18 hr. The reaction was diluted with DCM and water (4 mL) and the layers were separated. The aqueous layer was extracted again with DCM (2×4 mL). The combined organic layer was washed with brine and dried over sodium sulfate. Flash column chromatography (automated system, 12 g silica, 0-50% EA in Hexanes) provided tert-butyl N-methyl-N-[2-[1-methyl-5-(1-tetrahydropyran-2-yl-3-vinyl-pyrazolo[3,4-c]pyridin-5-yl)pyrazol-4-yl]oxyethyl]carbamate (29 mg, 0.060 mmol, 16% yield).

Step 5. To tert-butyl N-methyl-N-[2-[1-methyl-5-(1-tetrahydropyran-2-yl-3-vinyl-pyrazolo[3,4-c]pyridin-5-yl)pyrazol-4-yl]oxyethyl]carbamate (49.5 mg, 0.103 mmol) in chloroform (1 mL) was added TMSI (21.64 mg, 0.108 mmol, 15.0 μL), and the mixture was stirred at 22° C. for 18 hr. The reaction was then diluted with DCM and water (4 mL), and the layers were separated. The aqueous layer was extracted again with DCM (2×4 mL). The combined organic layer was washed with brine and dried over sodium sulfate. Flash column chromatography (automated system, 12 g silica, 0-25% MeOH in DCM with TEA) provided N-methyl-2-[1-methyl-5-(1-tetrahydropyran-2-yl-3-vinyl-pyrazolo[3,4-c]pyridin-5-yl)pyrazol-4-yl]oxy-ethanamine (12.5 mg, 0.033 mmol, 54.39% yield).

Step 6. To 2-[5-(bromomethyl)-3-ethoxy-4-iodo-pyrazol-1-yl]ethoxy-tert-butyl-dimethyl-silane (43 mg, 0.089 mmol) in acetonitrile (0.5 mL) was added K₂CO₃ (30.35 mg, 0.220 mmol) followed by N-methyl-2-[1-methyl-5-(1-tetrahydropyran-2-yl-3-vinyl-pyrazolo[3,4-c]pyridin-5-yl)pyrazol-4-yl]oxy-ethanamine, which was prepared according to the method described in Ex. 1, (28 mg, 0.073 mmol), and the mixture was stirred at 22° C. for 18 hr. The reaction mixture was cooled and diluted with DCM (5 mL), then filtered. The filtrate was worked up with DCM and water (5 mL). After separation of layers, the aqueous layer was extracted with DCM again (2×5 mL volume). The combined organic layer was washed with brine and then dried over sodium sulfate. Flash column chromatography (automated system, 12 g silica, 50-100% EA in Hexanes) provided N-[[2-[2-[tert-butyl(dimethyl)silyl]oxyethyl]-5-ethoxy-4-iodo-pyrazol-3-yl]methyl]-N-methyl-2-[1-methyl-5-(1-tetrahydropyran-2-yl-3-vinyl-pyrazolo[3,4-c]pyridin-5-yl)pyrazol-4-yl]oxy-ethanamine (42.4 mg, 0.054 mmol, 73.24% yield). LCMS: 791.4 (M+1).

Step 7. To a solution of the product from Step 6 (42.4 mg, 0.054 mmol) in anhydrous DMF (524 μL) was added sodium bicarbonate (13.51 mg, 0.161 mmol), and TBAC (16.4 mg, 0.059 mmol). The mixture was stirred while Argon was bubbled through, and palladium acetate (12 mg, 54 mol) was added. Argon was bubbled through for an additional 5 minutes. The vessel was sealed, and the reaction heated to 140° C. for 1.5 hr. The reaction was worked up with water (5 mL) and extracted with DCM (3×5 mL), combined organics were dried with Na₂SO₄ and concentrated under reduced pressure. Flash column chromatography provided (17E)-14-(2-{[tert-butyl(dimethyl)silyl]oxy}ethyl)-16-ethoxy-6,12-dimethyl-2-(oxan-2-yl)-2,10,11,12,13,14-hexahydro-6H-5,3-(azenometheno)tripyrazolo[3,4-f:3′,4′-j:3″,4″-n][1,4]oxazacyclopentadecine (3.7 mg, 0.0056 mmol, 10.41% yield). LCMS: 663.5 (M+1).

Step 8. To a solution of the product from Step 7 (3.7 mg, 0.006 mmol) in DCM (1.0 mL) was added HCl (4.0 M in 1,4-dioxane) (0.5 mL), and the reaction mixture was stirred at 22° C. for 1.5 hr. Volatiles were removed under reduced pressure, and the residue was purified by preparative HPLC (C18, 10-100% acetonitrile in water with 0.035% TFA) to afford Ex. 104 (1.49 mg, 0.003 mmol, 49.9% yield). ¹H NMR for Ex. 104 can be found in the below NMR table.

### Preparation of 1-[(10S,17E)-16-ethoxy-14-[(2S)-1-hydroxypropan-2-yl]-6,8,10-trimethyl-2,8,10,11,13,14-hexahydro-12H-5,3-(azenometheno)tripyrazolo[3,4-f:3′,4′-j:4″,3″-n][1,4]oxazacyclopentadecin-12-yl]ethan-1-one (Ex. 112)

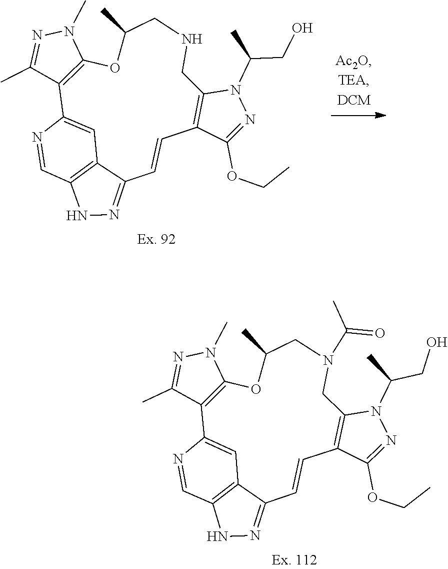 [SMILES: CCOc1nn([C@@H](C)CO)c2c1/C=C/c1n[nH]c3cnc(cc13)-c1c(C)nn(C)c1O[C@@H](C)CNC2 ; CCOc1nn([C@@H](C)CO)c2c1/C=C/c1n[nH]c3cnc(cc13)-c1c(C)nn(C)c1O[C@@H](C)CN(C(C)=O)C2]

To a solution of Ex. 92 (4.68 mg, 0.0095 mmol) in DCM (1 mL) was added acetic anhydride (10.82 mg, 0.106 mmol, 10 μL), followed by TEA (14.52 mg, 0.143 mmol, 20 μL). After the reaction mixture was stirred at RT for 2 hr, volatiles were removed and the residue was purified by flash column chromatography (automated system, 12 g silica, 0-12.5% Methanol in DCM, injected with 200 μL of TEA) followed by preparative HPLC (C18, 30-70% acetonitrile in water with 0.035% TFA), then again by flash chromatography (automated system, 12 g silica, 0-10% Methanol in DCM to afford Ex. 112 (1.65 mg, 32% yield). ¹H NMR for Ex. 112 can be found in the below NMR table.

### Preparation of 1-[(10S,17E)-16-ethoxy-14-[(2S)-1-hydroxypropan-2-yl]-6,8,10-trimethyl-2,8,10,11,13,14-hexahydro-12H-5,3-(azenometheno)tripyrazolo[3,4-f:3′,4′-j:4″,3″-n][1,4]oxazacyclopentadecin-12-yl]prop-2-en-1-one (Ex. 115)

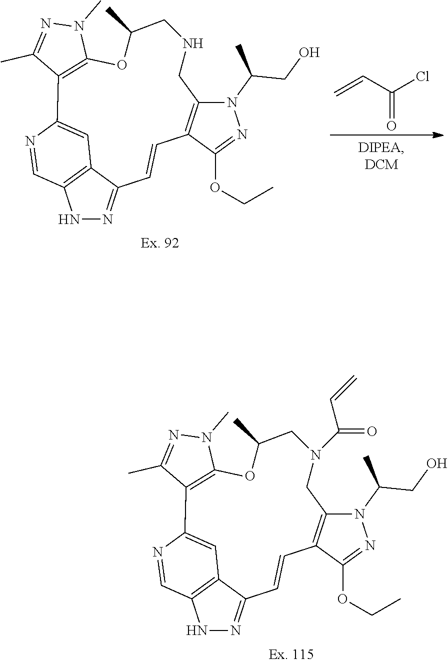

To a solution of Ex. 92 (10 mg, 0.020 mmol) in DCM (200 L) was added DIPEA (0.102 mmol, 118 μL) followed by prop-2-enoyl chloride (2.02 mg, 0.022 mmol). After the reaction mixture was stirred at RT for 2 hr. volatiles were removed and the residue was purified by flash column chromatography (automated system, 12 g silica, 0-12.5% Methanol in DCM, injected with 200 μL of TEA) followed by preparative HPLC (C18, 30-70% acetonitrile in water with 0.035% TFA), then again by flash chromatography (automated system, 12 g silica, 0-10% Methanol in DCM to afford Ex. 115 (2.54 mg). ¹H NMR for Ex. 115 can be found in the below NMR table.

### Preparation of (10S,17E)-16-ethoxy-6,8,10,12-tetramethyl-14-[(2S)-1-(pyrrolidin-1-yl)propan-2-yl]-2,10,11,12,13,14-hexahydro-8H-5,3-(azenometheno)tripyrazolo[3,4-f:3′,4′-j:4″,3″-n][1,4]oxazacyclopentadecine (Ex. 116)

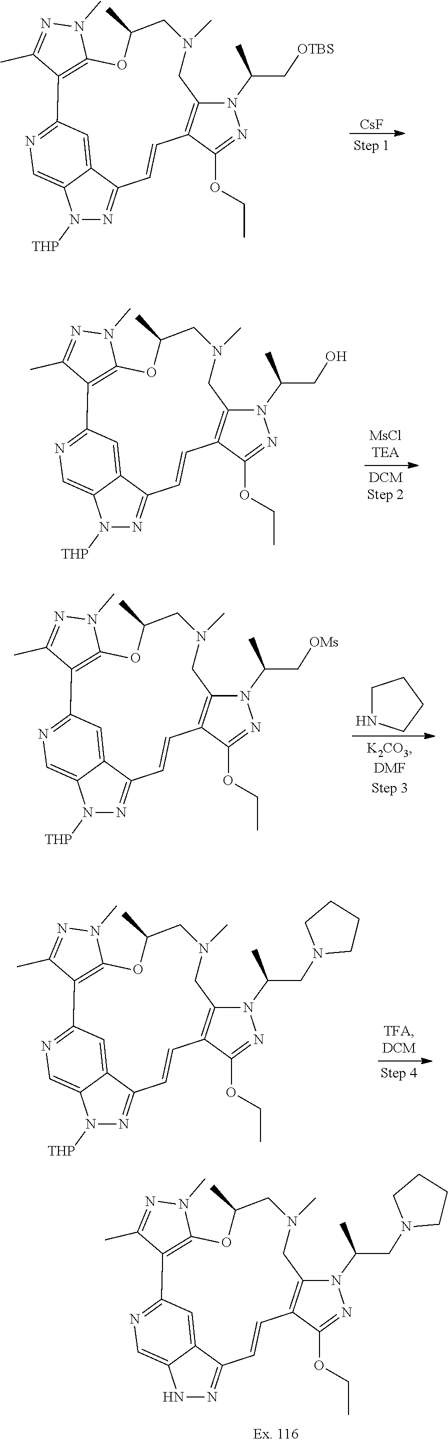

Step 1. The TBS deprotection of (10S,17E)-14-[(2S)-1-{[tert-butyl(dimethyl)silyl]oxy}propan-2-yl]-16-ethoxy-6,8,10,12-tetramethyl-2-(oxan-2-yl)-2,10,11,12,13,14-hexahydro-8H-5,3-(azenometheno)tripyrazolo[3,4-f:3,4′-j:4″,3″-n][1,4]oxazacyclopentadecine, which was prepared according to the method described in Ex. 27, was carried out using CsF in DMF to afford (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propan-1-ol.

Step 2. To a solution of the product from Step 1 (2 g, 3.39 mmol) in DCM (15.5 mL) was added triethylamine (685.2 mg, 6.77 mmol, 943.8 μL) and methanesulfonyl chloride (775.7 mg, 6.77 mmol, 525 L) at 0° C. The solution was stirred at 23° C. for 2 hr. After completion of the reaction, the mixture was poured into water (20 mL) and extracted with DCM (3×50 mL). The combined organic phases were washed with water (2×10 mL), brine, dried over anhydrous sodium sulfate, filtered and the solvent was removed by rotary evaporation to afford (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate (2.25 g, 3.37 mmol, 99.5% yield) as a yellow solid, which was carried over to the next step without further purification. LCMS: (M+1:689.5).

Step 3. To a solution of the mesylate from Step 2 (65 mg, 0.097 mmol) in dry DMF (0.364 mL) under Argon was added pyrrolidine (103.4 mg, 1.46 mmol, 0.121 mL) and anhydrous potassium carbonate (134.3 mg, 971.9 μmol). The mixture was stirred under Argon at 60° C. for 20 hr. The reaction mixture was diluted with water (1 mL) and DCM (3 mL), and the aqueous phase was extracted with DCM (3×3 mL). The combined organic layers were washed with brine, dried over anhydrous sodium sulfate, filtered and concentrated under reduced pressure to obtain (10S,17E)-16-ethoxy-6,8,10,12-tetramethyl-2-(oxan-2-yl)-14-[(2S)-1-(pyrrolidin-1-yl)propan-2-yl]-2,10,11,12,13,14-hexahydro-8H-5,3-(azenometheno)tripyrazolo[3,4-f:3′,4′-j:4″,3″-n][1,4]oxazacyclopentadecine (61 mg) as a white solid. LCMS: (M+1:644.5).

Step 4. To a solution of the product from Step 3 (62 mg, 0.096 mmol) in DCM (0.6 mL) at 0° C. was added TFA (219.6 mg, 1.93 mmol, 0.147 mL) and the reaction mixture stirred for 15 hr. The volatiles were evaporated in vacuo and the residue was purified using preparative HPLC (C18, 40-50% MeCN in water with 0.035% TFA) to provide Ex. 116 (28.67 mg) as yellow gum. ¹H NMR for Ex. 116 can be found in the below NMR table.

### Preparation of (10S,17E)-14-[(2S)-1-(azetidin-1-yl)propan-2-yl]-16-ethoxy-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecine (Ex. 117)

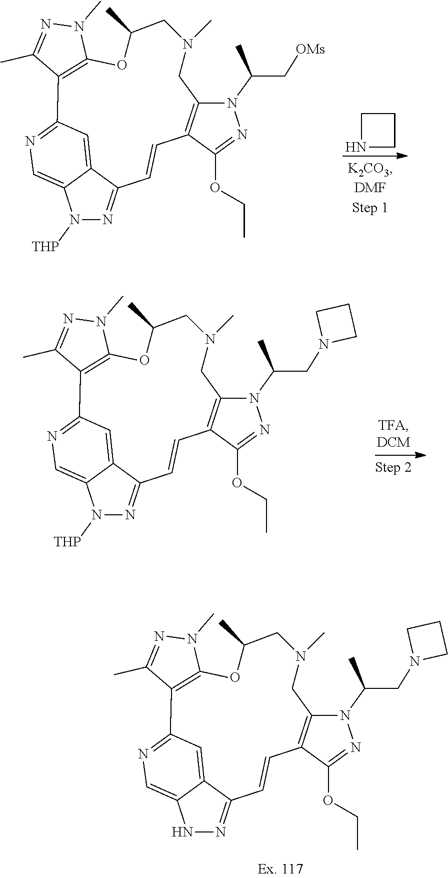

Ex. 117 was prepared according to the same method as described in steps 3 and 4 of Ex. 116 starting from (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using azetidine instead of pyrrolidine. ¹H NMR for Ex. 117 can be found in the below NMR table.

### Preparation of (10S,17E)-16-ethoxy-6,8,10,12-tetramethyl-14-[(2S)-1-(morpholin-4-yl)propan-2-yl]-2,10,11,12,13,14-hexahydro-8H-5,3-(azenometheno)tripyrazolo[3,4-f:3′,4′-j:4″,3″-n][1,4]oxazacyclopentadecine (Ex. 118)

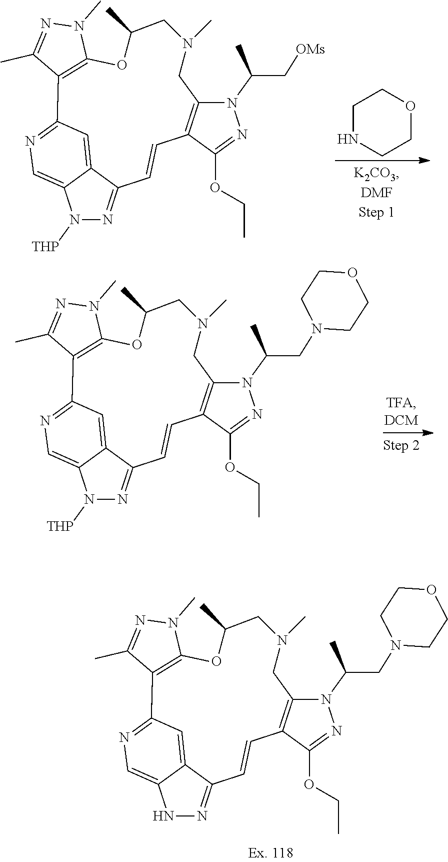

Ex. 118 was prepared according to the same method as described in steps 3 and 4 of Ex. 116 starting from (2S)-2-[I(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using morpholine instead of pyrrolidine. ¹H NMR for Ex. 118 can be found in the below NMR table.

### Preparation of (2S)-2-[(10S,17E)-16-ethoxy-6,8,10,12-tetramethyl-2,8,10,11,12,13-hexahydro-14H-5,3-(azenometheno)tripyrazolo[3,4-f3′,4′-j:4″,3″-n][1,4]oxazacyclo[pentadecin-14-yl]-N-methylpropan-1-amine (Ex. 119)

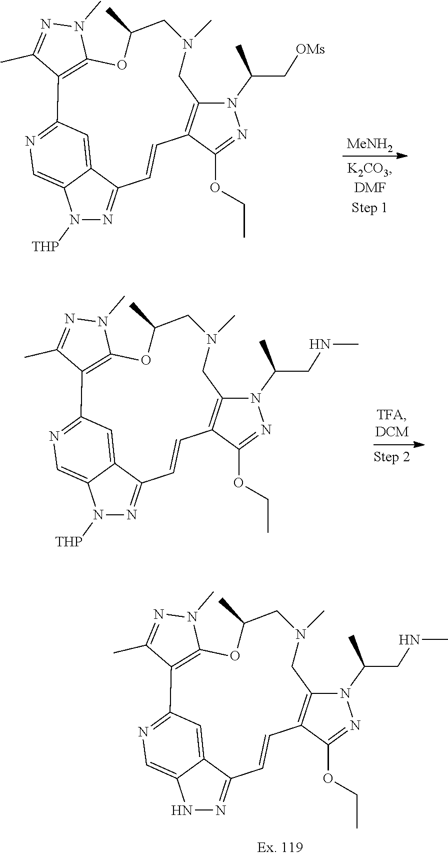

Ex. 119 was prepared according to the same method as described in steps 3 and 4 of Ex. 116 starting from (2S)-2-[I(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using methylamine instead of pyrrolidine. ¹H NMR for Ex. 119 can be found in the below NMR table.

### Preparation of (10S,17E)-16-ethoxy-6,8,10,12-tetramethyl-14-[(2S)-1-(piperidin-1-yl)propan-2-yl]-2,10,11,12,13,14-hexahydro-8H-5, 3-(azenometheno)tripyrazolo[3,4-f:3′,4′-j:4″,3″-n][1,4]oxazacyclopentadecine (Ex. 121)

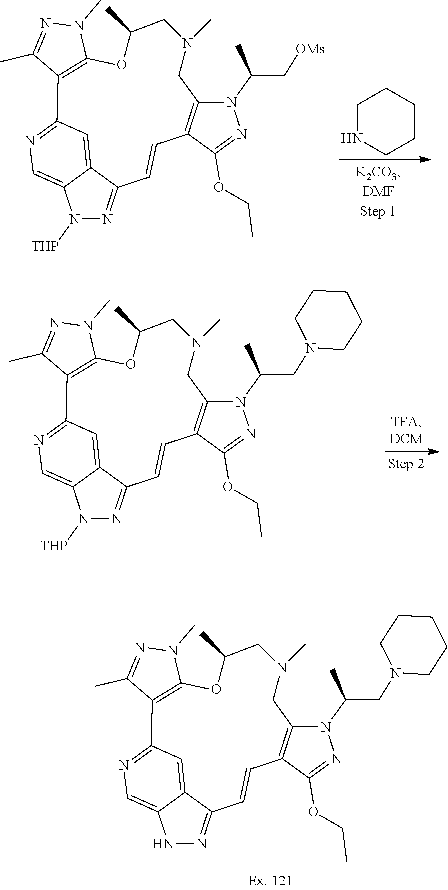

Ex. 121 was prepared according to the same method as described in steps 3 and 4 of Ex. 116 starting from (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using piperidine instead of pyrrolidine. 1H NMR for Ex. 121 can be found in the below NMR table.

### Preparation of (2S)-2-[(10S,17E)-16-ethoxy-6,8,10,12-tetramethyl-2,8,10,11,12,13-hexahydro-14H-5,3-(azenometheno)tripyrazolo[3,4-f3′,4′-j:4″,3″-n][1,4]oxazacyclopentadecin-14-yl]-N,N-dimethylpropan-1-amine (Ex. 122)

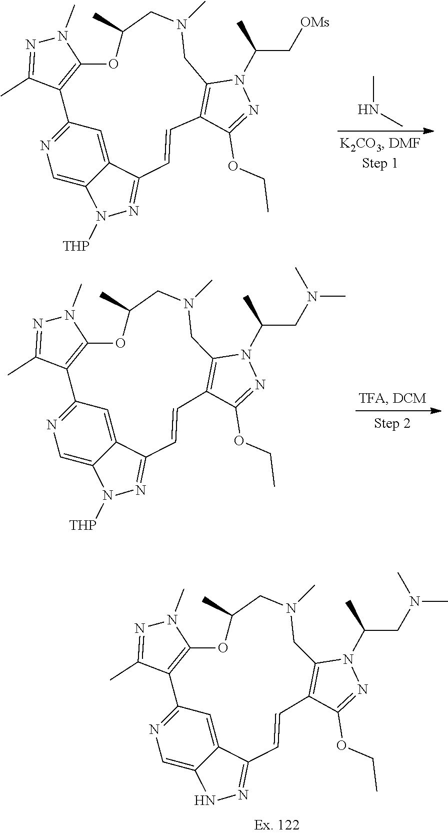

Ex. 122 was prepared according to the same method as described in steps 3 and 4 of Ex. 116 starting from (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using dimethylamine instead of pyrrolidine. ¹H NMR for Ex. 122 can be found in the below NMR table.

### Preparation of (3S)-1-{(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propyl}pyrrolidin-3-ol (Ex. 123)

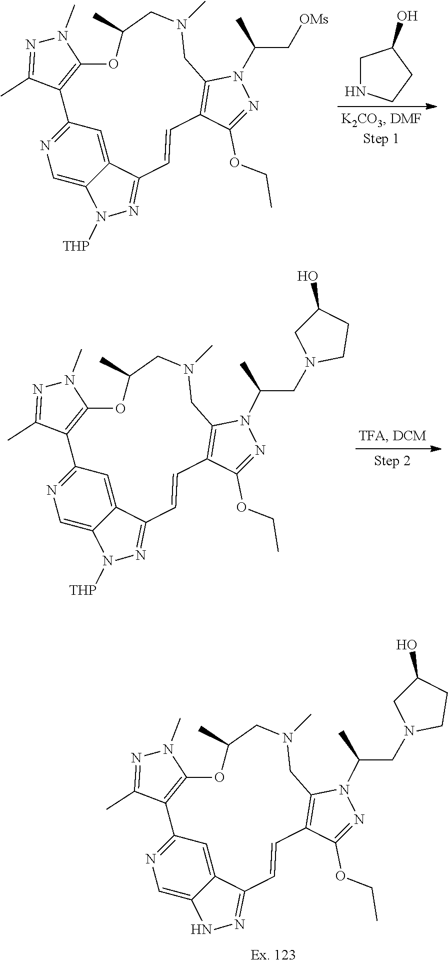

Ex. 123 was prepared according to the same method as described in steps 3 and 4 of Ex. 116 starting from (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using (S)-pyrrolidine-3-ol instead of pyrrolidine. ¹H NMR for Ex. 123 can be found in the below NMR table.

### Preparation of (3R)-1-{(2S)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propyl}pyrrolidin-3-ol (Ex. 124)

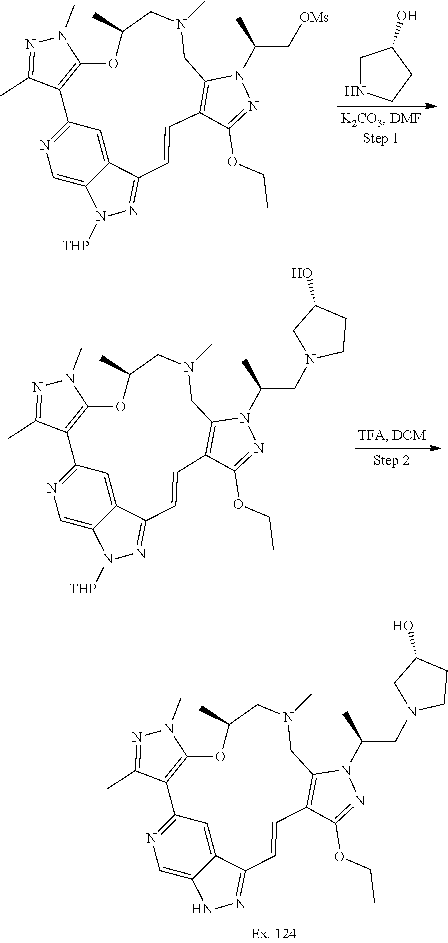

Ex. 124 was prepared according to the same method as described in steps 3 and 4 of Ex. 116 starting from (2S)-2-[(10S,17E)-16-ethoxy-6,8,10,12-tetramethyl-2-(oxan-2-yl)-10,11,12,13-tetrahydro-8H-5,3-(azenometheno)tripyrazolo[3,4-f:3′,4′-j:4″,3″-n][1,4]oxazacyclopentadecin-14(2H)-yl]propyl methanesulfonate using (R)-pyrrolidine-3-ol instead of pyrrolidine. ¹H NMR for Ex. 124 can be found in the below NMR table.

### Preparation of (10S,17E)-6,8,10,12,16,20-hexamethyl-14-[2-(pyrrolidin-1-yl)ethyl]-2,10,11,12,13,14-hexahydro-8H-5,3-(azenometheno)tripyrazolo[3,4-f:3′,4′-j:4″,3″-n][1,4]oxazacyclopentadecine (Ex. 125)

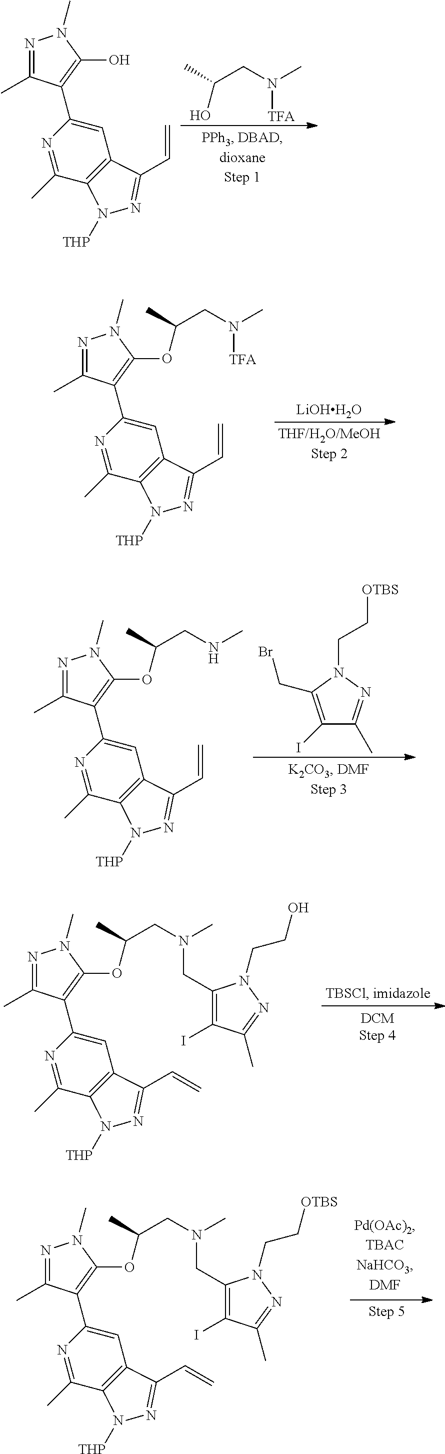

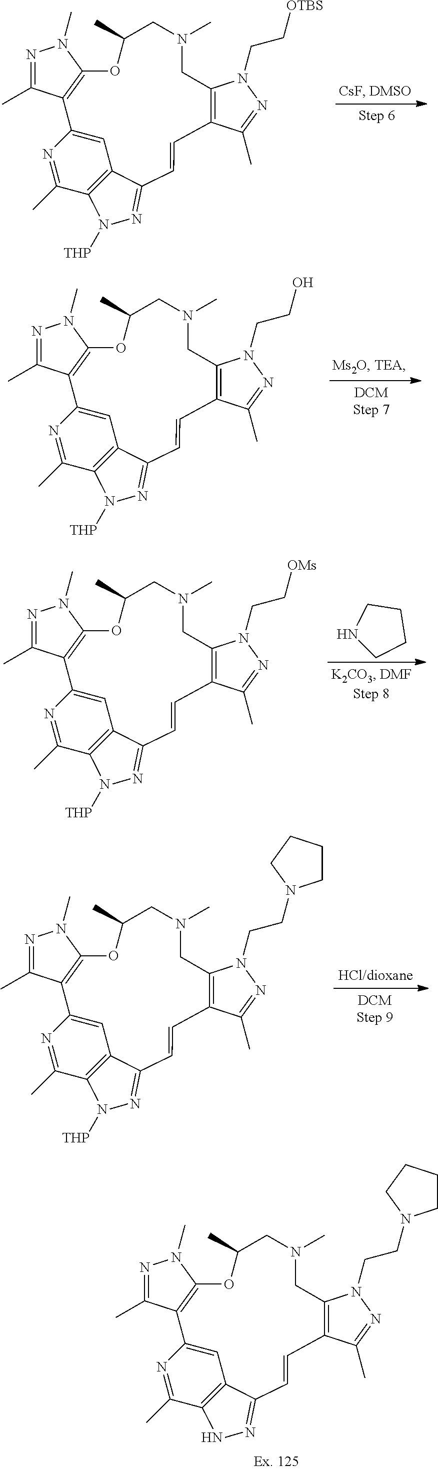

Step 1. A mixture of 2,5-dimethyl-4-(7-methyl-1-tetrahydropyran-2-yl-3-vinyl-pyrazolo[3,4-c]pyridin-5-yl)pyrazol-3-ol, which was prepared according to the method described in Ex. 47, (3.00 g, 8.49 mmol, 1 eq), commercially available 2,2,2-trifluoro-N-[(2R)-2-hydroxypropyl]-N-methyl-acetamide (2.36 g, 12.7 mmol, 1.5 eq), and PPh₃ (3.34 g, 12.7 mmol, 1.5 eq) in dioxane (100 mL) was degassed and purged with N₂ for 3 times, and DBAD (3.91 g, 16.9 mmol, 2 eq) was then added. The mixture was stirred at 0° C. for 2 hours under N₂ atmosphere. On completion, the mixture was filtered and concentrated to give a residue. The residue was purified by column chromatography (SiO₂, Petroleum ether/Ethyl acetate=1:0 to 0:1) to give N-((2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)propyl)-2,2,2-trifluoro-N-methylacetamide (1.30 g, 29% yield) as yellow oil. LCMS: (M+1: 521.4).

Step 2. To a solution of the product from Step 1 (1.30 g, 1.25 mmol, 1 eq) in THE (6 mL), MeOH (6 mL), H₂O (2 mL) was added LiOH·H₂O (157 mg, 3.75 mmol, 3 eq). The mixture was stirred at 25° C. for 14 hours. On completion, the mixture was filtered and concentrated to give a residue. The residue was purified by column chromatography (SiO₂, Petroleum ether/Ethyl acetate=1:0 to 0:1) to give (2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylpropan-1-amine (360 mg, 67% yield) as yellow oil. LCMS: (M+1: 425.2).

Step 3. To a solution of the product form Step 2 (340 mg, 0.800 mmol, 1 eq), 2-[5-(bromomethyl)-4-iodo-3-methyl-pyrazol-1-yl]ethoxy-tert-butyl-dimethyl-silane, which was prepared according to the method described in Ex. 13 and 14 (478 mg, 1.04 mmol, 1.3 eq) in DMF (8 mL) was added K₂CO₃ (332 mg, 2.40 mmol, 3 eq). The mixture was stirred at 80° C. for 0.5 hours. On completion, the reaction mixture was partitioned between ethyl acetate (30 mL×3) and water (30 mL), and the combined organic phase was dried over anhydrous sodium sulfate, filtered and concentrated to give a residue. The residue was purified by column chromatography (SiO₂, Petroleum ether/THF=1:0 to 0:1) to give 2-(5-((((2S)-2-((1,3-dimethyl-4-(7-methyl-1-(tetrahydro-2H-pyran-2-yl)-3-vinyl-H-pyrazolo[3,4-c]pyridin-5-yl)-1H-pyrazol-5-yl)oxy)propyl)(methyl)amino)methyl)-4-iodo-3-methyl-1H-pyrazol-1-yl)ethan-1-ol (280 mg, 50% yield) as yellow oil. LCMS: (M+1: 689.2).

Step 4. To a solution of the product from Step 3 (200 mg, 0.290 mmol, 1 eq) in DCM (2 mL) was added TBSCl (65.6 mg, 0.435 mmol, 1.5 eq) and imidazole (29.6 mg, 435 mol, 1.5 eq). The mixture was stirred at 25° C. for 2 hours. On completion, the mixture was filtered and concentrated to give a residue. The residue was purified by column chromatography (SiO₂, Petroleum ether/Ethyl acetate=1:0 to 0:1) to give (2S)-N-((1-(2-((tert-butyldimethylsilyl)oxy)ethyl)-4-iodo-3-methyl-1H-pyrazol-5-yl)methyl)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylpropan-1-amine (170 mg, 72% yield) as yellow oil. LCMS: (M+1: 803.3).

Step 5. A mixture of the product from Step 4 (150 mg, 186 μmol, 1 eq), Pd(OAc)₂ (8.39 mg, 37.3 μmol, 0.2 eq), TBAC (51.9 mg, 186 μmol, 1 eq) and NaHCO₃ (39.2 mg, 467 μmol, 2.5 eq) in DMF (1 mL) was degassed and purged with N₂ for 3 times, and then stirred at 130° C. for 0.5 hours under N₂ atmosphere. On completion, the reaction mixture was partitioned between ethyl acetate (10 mL×3) and water (3 mL). The combined organic phase was dried over anhydrous sodium sulfate, filtered, and concentrated to give a residue. The residue was purified by column chromatography (SiO₂, Petroleum ether/Ethyl acetate=1:0 to 0:1) to give (10S,17E)-14-(2-{[tert-butyl(dimethyl)silyl]oxy}ethyl)-6,8,10,12,16,20-hexamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (40.0 mg, 31% yield) as yellow oil. LCMS: (M+1: 675.4).

Step 6. To a solution of the product from Step 5 (40.0 mg, 59.2 μmol, 1 eq) in DMSO (1 mL) was added CsF (45.0 mg, 296 mol, 5 eq). The mixture was stirred at 25° C. for 1 hour. On completion, the reaction mixture was partitioned between ethyl acetate (10 mL×3) and water (4 mL), and the combined organic phase was dried over anhydrous sodium sulfate, filtered, and concentrated to give 2-[(10S,17E)-6,8,10,12,16,20-hexamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (30.0 mg, 90% yield) as yellow oil. LCMS: (M+1: 561.4).

Step 7. To a solution of the product from Step 6 (30.0 mg, 53.5 mol, 1 eq) in DCM (1 mL) was added TEA (16.2 mg, 160 mol, 3 eq) and methanesulfonic anhydride (23.3 mg, 134 μmol, 2.5 eq). The mixture was stirred at 25° C. for 1 hour. On completion, the reaction mixture was partitioned between dichloromethane (5 mL×3) and water (3 mL), and the combined organic phase was dried over anhydrous sodium sulfate, filtered, and concentrated to give 2-[(10S,17E)-6,8,10,12,16,20-hexamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethyl methanesulfonate (30.0 mg, 87% yield) as yellow oil. LCMS: (M+1: 639.4).

Step 8. To a solution of the product from Step 7 (30.0 mg, 46.9 mol, 1 eq) in DMF (1 mL) was added K₂CO₃ (19.4 mg, 140 μMol, 3 eq) and pyrrolidine (3.34 mg, 46.9 mol, 1 eq). The mixture was stirred at 80° C. for 1 hour. On completion, the reaction mixture was partitioned between ethyl acetate (10 mL×3) and water (5 mL), and the combined organic phase was dried over anhydrous sodium sulfate, filtered and concentrated to give (10S,17E)-6,8,10,12,16,20-hexamethyl-2-(oxan-2-yl)-14-[2-(pyrrolidin-1-yl)ethyl]-2,10,11,12,13,14-hexahydro-8H-5,3-(azenometheno)tripyrazolo[3,4-f:3′,4′-j:4″,3″-n][1,4]oxazacyclopentadecine (28.0 mg, 97% yield) as yellow oil. LCMS: (M+1: 614.4).

Step 9. To a solution of the product form Step 8 (25.0 mg, 40.7 μmol, 1 eq) in DCM (0.5 mL) was added HCl/dioxane (2 M, 1.25 mL, 61 eq). The mixture was stirred at 25° C. for 0.2 hours. On completion, the mixture was filtered and concentrated to give a residue. The crude product was purified by reversed-phase HPLC (column: Waters xbridge 150*25 mm 10 um; mobile phase: [water(NH4HCO3)-ACN]; gradient:18%-48% B over 10 min]; B %: 10%-40%,10 min) to give Ex. 125 (1.26 mg) as a white solid. ¹H NMR for Ex. 125 can be found in the below NMR table.

### Preparation of (10S,17E)-6,8,10,12,16,20-hexamethyl-15-[2-(pyrrolidin-1-yl)ethyl]-2,10,11,12,13,15-hexahydro-8H-5,3-(azenometheno)tripyrazolo[3,4-f:3′,4′-j:4″,3″-n][1,4]oxazacyclopentadecine (Ex. 126)

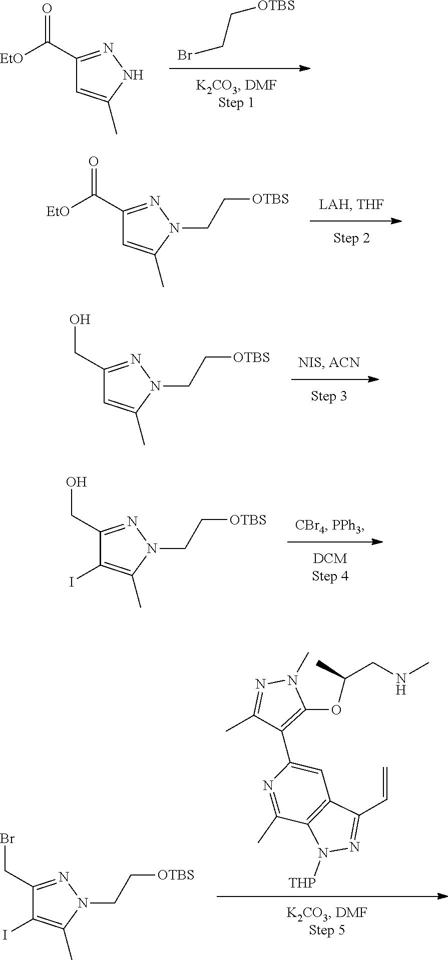

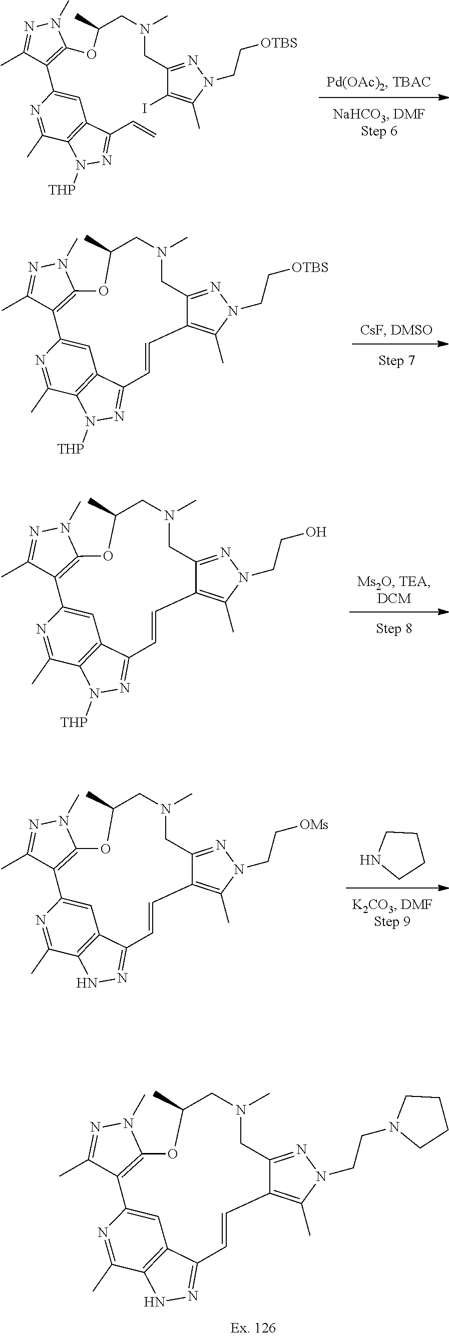

Step 1. To a solution of commercially available ethyl 5-methyl-1H-pyrazole-3-carboxylate (5.00 g, 32.4 mmol, 1 eq) and commercially available 2-bromoethoxy-tert-butyl-dimethyl-silane (11.6 g, 48.6 mmol, 1.5 eq) in DMF (100 mL) was added K₂CO₃ (8.96 g, 64.8 mmol, 2 eq). The mixture was stirred at 80° C. for 16 hours. On completion, the reaction mixture was partitioned between ethyl acetate (300 mL×3) and water (300 mL). The combined organic phase was dried over anhydrous sodium sulfate, filtered, and concentrated. The residue was purified by column chromatography (SiO₂, Petroleum ether/Ethyl acetate=1:0 to 0:1) to give ethyl 1-[2-[tert-butyl(dimethyl)silyl]oxyethyl]-5-methyl-pyrazole-3-carboxylate (5.00 g, 16.0 mmol, 49% yield) as yellow oil. LCMS: (M+1: 313.1).

Step 2. To a solution of ethyl 1-[2-[tert-butyl(dimethyl)silyl]oxyethyl]-5-methyl-pyrazole-3-carboxylate (5.00 g, 16.0 mmol, 1 eq) in THF (50 mL) was added LAH (2.5 M, 6.40 mL, 1 eq). The mixture was stirred at 0° C. for 0.5 hours. On completion, the mixture was quenched with water (7 mL), aqueous solution of NaOH (15%) (7 mL), water (21 mL) and extracted with ethyl acetate (50 mL×3). The combined organic phase was dried over anhydrous sodium sulfate, filtered, and concentrated to give [1-[2-[tert-butyl(dimethyl)silyl]oxyethyl]-5-methyl-pyrazol-3-yl]methanol (4.00 g, 92% yield) as yellow oil. LCMS: (M+1: 271.3).

Step 3. To a solution of [1-[2-[tert-butyl(dimethyl)silyl]oxyethyl]-5-methyl-pyrazol-3-yl]methanol (3.60 g, 13.3 mmol, 1 eq) in ACN (4 mL) was added NIS (3.59 g, 15.9 mmol, 1.2 eq). The mixture was stirred at 0° C. for 2 hours. On completion, the mixture was quenched with saturated sodium sulfite solution (2 mL) and extracted with ethyl acetate (10 mL×3). The combined organic phase was dried over anhydrous sodium sulfate, filtered, and concentrated. The residue was purified by column chromatography (SiO₂, Petroleum ether/Ethyl acetate=1:0 to 0:1) to give [1-[2-[tert-butyl(dimethyl)silyl]oxyethyl]-4-iodo-5-methyl-pyrazol-3-yl]methanol (4.00 g, 75% yield) as yellow oil. LCMS: (M+1: 397.0).

Step 4. To a solution of [1-[2-[tert-butyl(dimethyl)silyl]oxyethyl]-4-iodo-5-methyl-pyrazol-3-yl]methanol (3.90 g, 9.84 mmol, 1 eq), and CBr₄ (3.92 g, 11.8 mmol, 1.2 eq) in DCM (100 mL) was added PPh₃ (3.87 g, 14.7 mmol, 1.5 eq). The mixture was stirred at 25° C. for 0.5 hours. On completion, the mixture was filtered, and the filtrate concentrated to give a residue. This residue was purified by column chromatography (SiO₂, Petroleum ether/Ethyl acetate=1:0 to 0:1) to give 2-[3-(bromomethyl)-4-iodo-5-methyl-pyrazol-1-yl]ethoxy-tert-butyl-dimethyl-silane (2.70 g, 5.88 mmol, 59% yield) as yellow oil. LCMS: (M+1: 460.9).

Step 5. To a solution of (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 47, (300 mg, 0.706 mmol, 1 eq) and 2-[3-(bromomethyl)-4-iodo-5-methyl-pyrazol-1-yl]ethoxy-tert-butyl-dimethyl-silane (356 mg, 0.777 mmol, 1.1 eq) in DMF (6 mL) was added K₂CO₃ (293 mg, 2.12 mmol, 3 eq). The mixture was stirred at 25° C. for 0.5 hours. On completion, the mixture was filtered and concentrated. The residue was purified by column chromatography (SiO₂, Petroleum ether/Ethyl acetate=1:0 to 0:1) to give (2S)-N-[[1-[2-[tert-butyl(dimethyl)silyl]oxyethyl]-4-iodo-5-methyl-pyrazol-3-yl]methyl]-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine (300 mg, 52% yield) as yellow oil. LCMS: (M+1: 803.3).

Step 6. A mixture of the product from Step 5 (270 mg, 336 μmol, 1 eq), Pd(OAc)₂ (15.1 mg, 67.2 mol, 0.2 eq), TBAC (93.4 mg, 336 μmol, 1 eq) and NaHCO₃ (70.6 mg, 840 mol, 2.5 eq) in DMF (3 mL) was degassed and purged with N₂ for 3 times, and then the mixture was stirred at 120° C. for 0.5 hours under N₂ atmosphere. On completion, the reaction mixture was partitioned between ethyl acetate (10 mL×3) and water (15 mL). The combined organic phase was dried over anhydrous sodium sulfate, filtered, and concentrated. The residue was purified by column chromatography (SiO₂, Petroleum ether/Ethyl acetate=1:0 to 0:1) to give (10S,17E)-15-(2-{[tert-butyl(dimethyl)silyl]oxy}ethyl)-6,8,10,12,16,20-hexamethyl-2-(oxan-2-yl)-2,10,11,12,13,15-hexahydro-8H-5,3-(azenometheno)tripyrazolo|3,4-f:3′,4′-j:4″,3″-n][1,4]oxazacyclopentadecine (140 mg, 61% yield) as yellow oil. LCMS: (M+1: 675.5).

Step 7. To a solution of the product from Step 6 (90.0 mg, 0.133 mmol, 1 eq) in DMSO (1 mL) was added CsF (101 mg, 0.666 mmol, 5 eq). The mixture was stirred at 25° C. for 0.5 hours. On completion, the reaction mixture was partitioned between ethyl acetate (5 mL×3) and water (5 mL). The combined organic phase was dried over anhydrous sodium sulfate, filtered and concentrated to give 2-[(10S,17E)-6,8,10,12,16,20-hexamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (70.0 mg, 124 μmol, 93% yield) as yellow oil. LCMS: (M+1: 561.3).

Step 8. To a solution of the product from Step 7 (70.0 mg, 124 mol, 1 eq) in DCM (1 mL) was added TEA (37.9 mg, 0.374 mmol, 3 eq) and methylsulfonyl methanesulfonate (54.3 mg, 0.312 mmol, 2.5 eq). The mixture was stirred at 25° C. for 0.5 hours. On completion, the reaction mixture was partitioned between dichloromethane (5 mL×3) and water (5 mL). The combined organic phase was dried over anhydrous sodium sulfate, filtered and concentrated to give 2-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethyl methanesulfonate (50.0 mg, 72% yield) as yellow oil. LCMS: (M+1: 555.2).

Step 9. To a solution of the product from Step 8 (50.0 mg, 0.0901 mmol, 1 eq) and pyrrolidine (6.41 mg, 0.0901 mmol, 1 eq) in DMF (1 mL) was added K₂CO₃ (37.3 mg, 0.270 mmol, 3 eq). The mixture was stirred at 80° C. for 1 hour. The mixture was stirred at 25° C. for 0.2 hours. On completion, the mixture was filtered and concentrated to give a residue. The crude product was purified by reversed-phase HPLC(column: Phenomenex luna C18 150*25 mm*10 um; mobile phase: [water(FA)-ACN]; gradient:0%-20% B over 10 min) to afford Ex. 126 (2.95 mg, 0.00543 mmol, 6.02% yield, 97.5% purity) as a white solid. ¹H NMR for Ex. 126 can be found in the below NMR table.

### Preparation of 1-[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]ethan-1-one (Ex. 131)

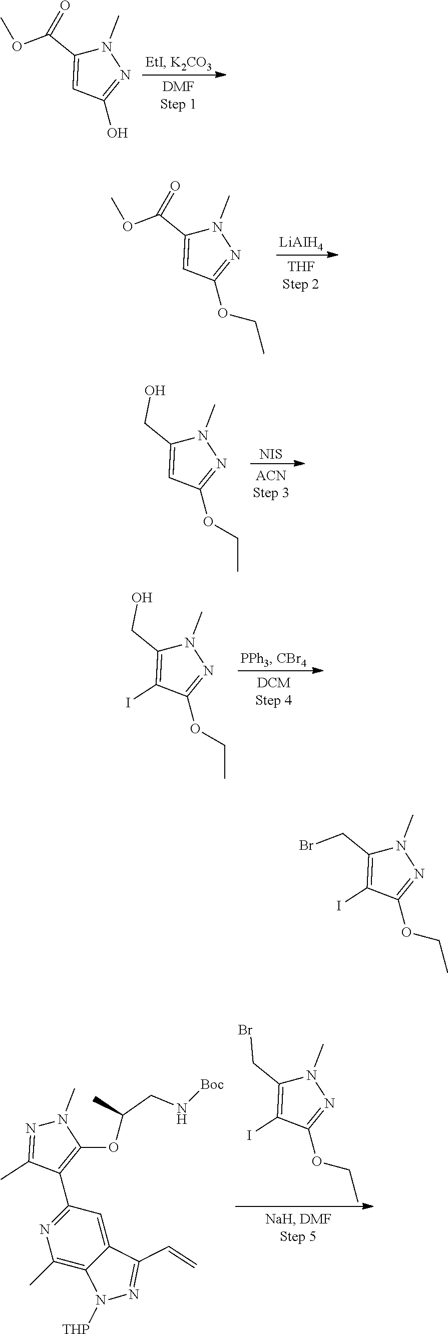

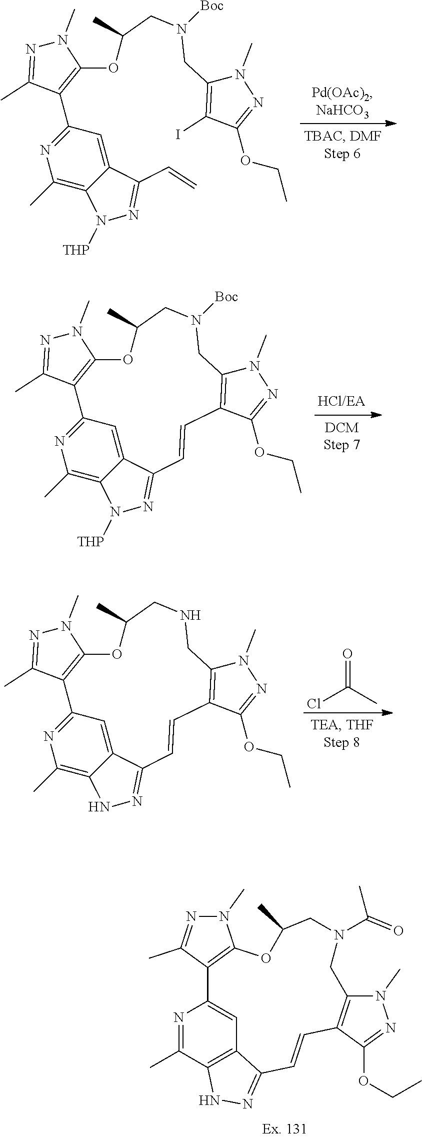

Step 1. To a solution of commercially available methyl 5-hydroxy-2-methyl-pyrazole-3-carboxylate (9 g, 57.6 mmol, 1 eq), EtI (8.99 g, 57.6 mmol, 4.61 mL, 1 eq) in DMF (90 mL) was added K₂CO₃ (23.9 g, 172 mmol, 3 eq). The mixture was stirred at 80° C. for 2 hrs. On completion, the mixture was diluted with water (150 mL) and extracted with ethyl acetate (40 mL×3). The combined organic phase was washed with water (200 mL×2), dried over Na₂SO₄, filtered and the filtrate was concentrated to give a residue. The residue was purified by column chromatography (SiO₂, Petroleum ether/Ethyl acetate=1:0 to 1:1) to give 5-(hydroxymethyl)-1-methyl-1H-pyrazol-3-ol (10 mg, 94% yield) as an oil. ¹H NMR (400 MHz, CDCl₃) δ=6.15-6.12 (m, 1H), 4.17-4.09 (m, 2H), 4.01-3.98 (m, 3H), 3.84-3.81 (m, 3H), 1.37-1.32 (m, 3H). LCMS: (M+1:185.2).

Step 2. To a solution of methyl 5-ethoxy-2-methyl-pyrazole-3-carboxylate (9 g, 48.8 mmol, 1 eq) in THE (90 mL) was added LiAlH₄ (1.85 g, 48.8 mmol, 1 eq) at 0° C. The mixture was stirred at 0° C. for 2 h. On completion, the mixture was quenched with MeOH (200 mL) at 0° C., and the mixture was concentrated to give a residue. The residue was purified by column chromatography (SiO₂, DCM/MeOH=1:0 to 1:1) to give (3-ethyl-1-methyl-1H-pyrazol-5-yl)methanol (7.9 g, 98% yield) as a white solid. LCMS: (M+1:157.4).

Step 3. To a solution of (5-ethoxy-2-methyl-pyrazol-3-yl)methanol (6.9 g, 44.1 mmol, 1 eq) in ACN (70 mL) was added NIS (8.95 g, 39.7 mmol, 0.9 eq) at 0° C. The mixture was stirred at 0° C. for 2 h. On completion, the mixture was quenched with aqueous saturated solution of Na₂SO₃ (100 mL) and extracted with ethyl acetate (30 mL×3). The combined organic phase was dried over anhydrous sodium sulfate, filtered and concentrated to give a residue. The residue was purified by column chromatography (SiO₂, Petroleum ether/Ethyl acetate=1:0 to 1:1) to give (3-ethyl-4-iodo-1-methyl-1H-pyrazol-5-yl)methanol (14.5 g, 43.6 mmol, 99% yield) as white solid. LCMS: (M+1:283.0).

Step 4. To a solution of (5-ethoxy-4-iodo-2-methyl-pyrazol-3-yl)methanol (12.5 g, 44.3 mmol, 1 eq) and PPh₃ (13.9 g, 53.1 mmol, 1.2 eq) in DCM (120 mL) was added CBr₄ (17.6 g, 53.1 mmol, 1.2 eq) at 0° C. The mixture was stirred at 0° C. for 2 h. On completion, the mixture was diluted with water (200 mL) and extracted with DCM (50 mL×3). The combined organic phase was dried over Na₂SO₄, filtered and the filtrate was concentrated to give a residue. The residue was purified by column chromatography (SiO₂, Petroleum ether/Ethyl acetate=1:0 to 65:35) to give 5-(bromomethyl)-3-ethoxy-4-iodo-1-methyl-1H-pyrazole (8.6 g, 56% yield) as yellow solid. ¹H NMR (400 MHz, CDCl₃) δ=4.40 (s, 2H), 4.28-4.20 (m, 2H), 3.80 (s, 3H), 1.40 (t, J=7.2 Hz, 3H). LCMS: (M+1:346.8).

Step 5. To a solution of tert-butyl N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]carbamate, which was prepared according to the method described in Ex. 47, (850 mg, 0.832 mmol, 1 eq) in DMF (10 mL) was added NaH (83.2 mg, 2.08 mmol, 60% purity, 2.5 eq) at 0° C. The mixture was stirred at 0° C. for 0.5 h, and then 5-(bromomethyl)-3-ethoxy-4-iodo-1-methyl-1H-pyrazole (287 mg, 0.832 mmol, 1 eq) was added at 0° C., and the mixture was stirred at 25° C. for 0.5 h. On completion, the reaction mixture was mixed with saturated solution of NH₄Cl (20 mL), and then partitioned between ethyl acetate (20 mL×3) and water (50 mL). The combined organic phase was washed with brine (100 mL), dried over anhydrous sodium sulfate, filtered, and concentrated to give a residue. The residue was purified by column chromatography (SiO₂, Petroleum ether/Ethyl acetate=1:0 to 0:1) to give tert-butyl N-[(2S)-2-[2,5-dimethyl-4-(7-methyl-1-tetrahydropyran-2-yl-3-vinyl-pyrazolo[3,4-c]pyridin-5-yl)pyrazol-3-yl]oxypropyl]-N-[(5-ethoxy-4-iodo-2-methyl-pyrazol-3-yl)methyl]carbamate (530 mg, 82% yield) as yellow solid. LCMS: (M+1=775.5).

Step 6. A mixture of the product form Step 5 (480 mg, 0.620 mmol, 1 eq), TBAC (172 mg, 0.620 mmol, 1 eq), NaHCO₃ (130 mg, 1.55 mmol, 2.5 eq) and Pd(OAc)₂ (27.8 mg, 0.124 mmol, 0.2 eq) in DMF (5 mL) was degassed and purged with N₂ for 3 times, and then the mixture was stirred at 130° C. for 2 h under N₂ atmosphere. On completion, the mixture was quenched with water (50 mL) and extracted with ethyl acetate (25 mL×3). The combined organic phase was dried over anhydrous sodium sulfate, filtered, and concentrated to give a residue. The residue was purified by column chromatography (SiO₂, Petroleum ether/THF=1:0 to 0:1) to give tert-butyl (10S,17E)-16-ethoxy-6,8,10,14,20-pentamethyl-2-(oxan-2-yl)-10,11,13,14-tetrahydro-8H-5,3-(azenometheno)tripyrazolo[3,4-f:3′,4′-j:4″,3″-n][1,4]oxazacyclopentadecine-12(2H)-carboxylate (280 mg, 70% yield) as yellow solid. LCMS: (M+1=647.4).

Step 7. To a solution of the product from Step 6 (260 mg, 402 mol, 1 eq) in DCM (2 mL) was added HCl/EtOAc (2 M, 201 μL, 1 eq). The mixture was stirred at 25° C. for 1 h. On completion, the reaction mixture was concentrated under reduced pressure to remove solvent and (10S,17E)-16-ethoxy-6,8,10,14,20-pentamethyl-2,10,11,12,13,14-hexahydro-8H-5,3-(azenometheno)tripyrazolo[3,4-f:3′,4′-j:4″,3″-n][1,4]oxazacyclopentadecine (200 mg, 99% yield) as yellow solid. LCMS: (M+1=463.3).

Step 8. To a solution of the product from Step 7 (100 mg, 0.216 mmol, 1 eq) in THE (1 mL) was added TEA (65.6 mg, 0.648 mmol, 3 eq) and acetyl chloride (17.0 mg, 0.216 mmol, 1 eq). The mixture was stirred at 25° C. for 1 h. On completion, the mixture was quenched with water (5 mL) and extracted with ethyl acetate (5 mL×3). The combined organic phase was dried over anhydrous sodium sulfate, filtered and concentrated to give a residue. The residue was purified by prep-HPLC (column: Phenomenex luna C18 150*25 mm*10 um; mobile phase: [water(FA)-ACN]; gradient:22%-52% B over 10 min) to give Ex. 131 (11.81 mg, 0.0011 mmol, 5.0% yield, 92.3% purity) as off-white solid. ¹H NMR for Ex. 131 can be found in the below NMR table.

### Preparation of (17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecine-16-carbonitrile (Ex. 150)

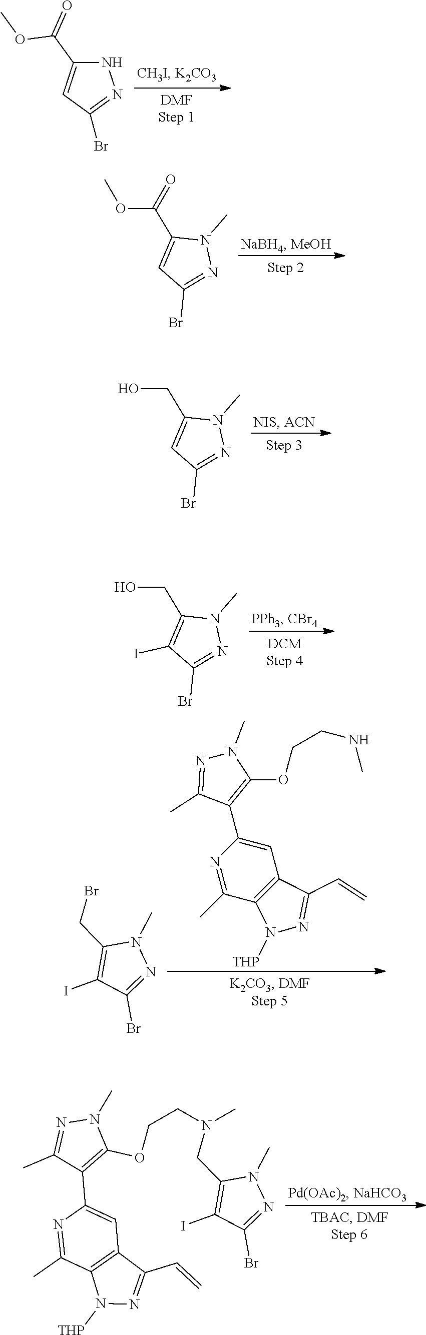

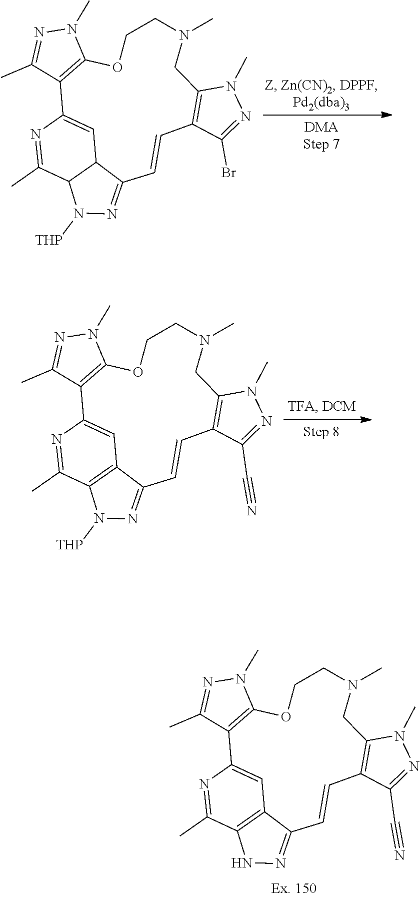

Step 1. To a solution of methyl 3-bromo-1H-pyrazole-5-carboxylate (1.00 g, 4.88 mmol, 1 eq) and iodomethane (3.46 g, 24.4 mmol, 5 eq) in DMF (10 mL) was added K₂CO₃ (1.01 g, 7.32 mmol, 1.5 eq). The mixture was stirred at 60° C. for 2 h. On completion, the mixture was diluted with water (30 mL) and extracted with ethyl acetate (10 mL×3). The combined organic phase was washed with water (20 mL×2) and dried over Na₂SO₄, filtered and the filtrate was concentrated to give a residue. The residue was purified by column chromatography (SiO₂, PE:THE=10:1) to give methyl 3-bromo-1-methyl-1H-pyrazole-5-carboxylate (720 mg, 3.29 mmol, 67% yield) as a white oil. LCMS: (M+1:219.0).

Step 2. To a solution of methyl 3-bromo-1-methyl-1H-pyrazole-5-carboxylate (720 mg, 3.29 mmol, 1 eq) in MeOH (7.2 mL) was added NaBH₄ (1.24 g, 32.9 mmol, 10 eq) at 0° C. The mixture was stirred at 25° C. for 1 h. On completion, the mixture was quenched by water (10 mL) at 0° C. and extracted with ethyl acetate (10 mL×3). The combined organic phase was washed with water (20 mL*2) and dried over Na₂SO₄, filtered and the filtrate was concentrated to give a residue. The residue was purified by column chromatography (SiO₂, PE:THF=5:1) to give (3-bromo-1-methyl-1H-pyrazol-5-yl)methanol (420 mg, 2.20 mmol, 67% yield) as a white solid. LCMS: (M+1:191.1).

Step 3. To a solution of (3-bromo-1-methyl-1H-pyrazol-5-yl)methanol (400 mg, 2.09 mmol, 1 eq) in ACN (4 mL) was added NIS (1.41 g, 6.28 mmol, 3 eq). The mixture was stirred at 40° C. for 1 h. On completion, the mixture was quenched with sat. Na₂SO₃ (10 mL). The mixture was separated, and the aqueous layer was extracted with EA (10 mL×3). The combined organic phase was dried over Na₂SO₄, filtered and the filtrate was concentrated to give a residue. The residue was purified by column chromatography (SiO₂, PE:THF=3:1) to give (3-bromo-4-iodo-1-methyl-1H-pyrazol-5-yl)methanol (560 mg, 1.77 mmol, 84% yield) as a white solid. LCMS: (M+1:318.8).

Step 4. To a solution of (3-bromo-4-iodo-1-methyl-1H-pyrazol-5-yl)methanol (530 mg, 1.67 mmol, 1 eq) in DCM (5 mL) was added PPh₃ (526 mg, 2.01 mmol, 1.2 eq), and CBr₄ (666 mg, 2.01 mmol, 1.2 eq) in DCM (2 mL) was added at 0° C. The mixture was stirred at 25° C. for 1 h. On completion, the mixture was concentrated to give a residue. The residue was purified by column chromatography (SiO₂, PE:THF=10:1) to give 3-bromo-5-(bromomethyl)-4-iodo-1-methyl-1H-pyrazole (580 mg, 1.53 mmol, 91% yield) as a white solid. LCMS: (M+1:380.7).

Step 5. To a solution of 3-bromo-5-(bromomethyl)-4-iodo-1-methyl-1H-pyrazole (249 mg, 0.655 mmol, 1 eq), 2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylethan-1-amine (269 mg, 0.655 mmol, 1 eq, which was prepared according to the method described in Ex. 140) in DMF (3 mL) was added K₂CO₃ (271 mg, 1.96 mmol, 3 eq). The mixture was stirred at 60° C. for 1 h. On completion, the mixture was diluted with water (10 mL) and extracted with ethyl acetate (5 mL×3). The combined organic phase was washed with water (20 mL×2) and dried over Na₂SO₄, filtered and the filtrate was concentrated to give a residue. The residue was purified by column chromatography (SiO₂, DCM:MeOH=15:1) to give N-((3-bromo-4-iodo-1-methyl-1H-pyrazol-5-yl)methyl)-2-((1,3-dimethyl-4-(7-methyl-2-(tetrahydro-2H-pyran-2-yl)-3-vinyl-2H-pyrazolo[3,4-c]pyridin-5-yl)-1H-pyrazol-5-yl)oxy)-N-methylethan-1-amine (415 mg, 0.585 mmol, 89% yield) as a yellow solid. LCMS: (M+1:709.0).

Step 6. To a solution of N-((3-bromo-4-iodo-1-methyl-1H-pyrazol-5-yl)methyl)-2-((1,3-dimethyl-4-(7-methyl-2-(tetrahydro-2H-pyran-2-yl)-3-vinyl-2H-pyrazolo[3,4-c]pyridin-5-yl)-1H-pyrazol-5-yl)oxy)-N-methylethan-1-amine (375 mg, 0.529 mmol, 1 eq) in DMF (20 mL) was added NaHCO₃ (111 mg, 1.32 mmol, 2.5 eq), TBAC (147 mg, 0.529 mmol, 1 eq) and Pd(OAc)₂ (23.7 mg, 0.106 mmol, 0.2 eq). The mixture was stirred at 130° C. for 1 h. On completion, the mixture was diluted with water (60 mL) and extracted with ethyl acetate (20 mL×3). The combined organic phase was washed with water (50 mL×2) and dried over Na₂SO₄, filtered and the filtrate was concentrated to give a residue. The residue was purified by column chromatography (SiO₂, PE:THF=5:1) to give (17E)-16-bromo-6,8,12,14,20-pentamethyl-1-(oxan-2-yl)-8,10,11,12,13,14-hexahydro-1H-5,3-(azenometheno)tripyrazolo[3,4-f:3′,4′-j:4″,3″-n][1,4]oxazacyclopentadecine (150 mg, 0.258 mmol, 49% yield) as a yellow solid. LCMS: (M+1:583.2).

Step 7. To a solution of (17E)-16-bromo-6,8,12,14,20-pentamethyl-1-(oxan-2-yl)-8,10,11,12,13,14-hexahydro-1H-5,3-(azenometheno)tripyrazolo[3,4-f:3′,4′-j:4″,3″-n][1,4]oxazacyclopentadecine (135 mg, 0.232 mmol, 1 eq) in DMA (1.5 mL) was added Zn (3.80 mg, 0.058 mmol, 0.25 eq), dicyanozinc (30.0 mg, 0.255 mmol, 1.1 eq) and DPPF (10.9 mg, 0.020 mmol, 0.085 eq), then Tris(dibenzylideneacetone)dipalladium(0) (7.02 mg, 0.0077 mmol, 0.033 eq) was added under N₂. The mixture was stirred at 120° C. for 16 h. On completion, the mixture was quenched with saturated NaCl (5 mL). The mixture was separated, and the aqueous layer was extracted with 2-MeTHF (3 mL×3). The combined organic phase was dried over Na₂SO₄, filtered and the filtrate was concentrated to give a residue to give (17E)-6,8,12,14,20-pentamethyl-1-(oxan-2-yl)-8,10,11,12,13,14-hexahydro-1H-5,3-(azenometheno)tripyrazolo[3,4-f:3′,4′-j:4″,3″-n][1,4]oxazacyclopentadecine-16-carbonitrile (163 mg, crude) as a yellow oil. LCMS: (M+1:528.3).

Step 8. To a solution of (17E)-6,8,12,14,20-pentamethyl-1-(oxan-2-yl)-8,10,11,12,13,14-hexahydro-1H-5,3-(azenometheno)tripyrazolo[3,4-f:3′,4′-j:4″,3″-n][1,4]oxazacyclopentadecine-16-carbonitrile (135 mg, 0.256 mmol, 1 eq) in DCM (1.5 mL) was added TFA (2.01 g, 17.6 mmol, 68.7 eq). The mixture was stirred at 25° C. for 1 h. On completion, the mixture was concentrated to give a residue. The residue was purified by prep-HPLC to give (17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecine-16-carbonitrile (7.54 mg, 0.017 mmol, 6.64% yield) as an off-white solid (Ex. 150). ¹H NMR for Ex. 150 can be found in the below NMR table.

General Method C: using Ex. 23 as example.

### Preparation of {[(10S,17E)-12-ethyl-14-(2-hydroxyethyl)-6,8,10-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 23)

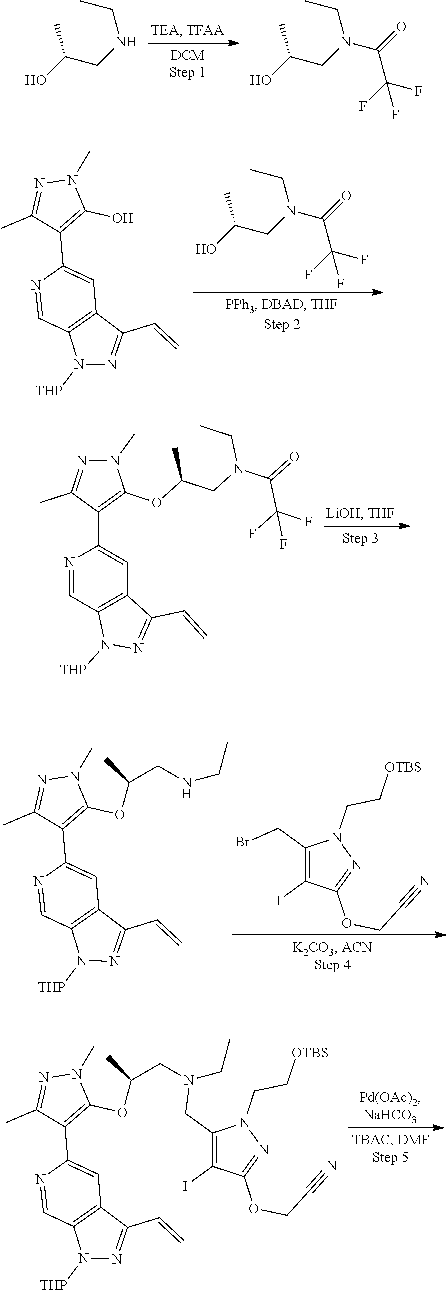

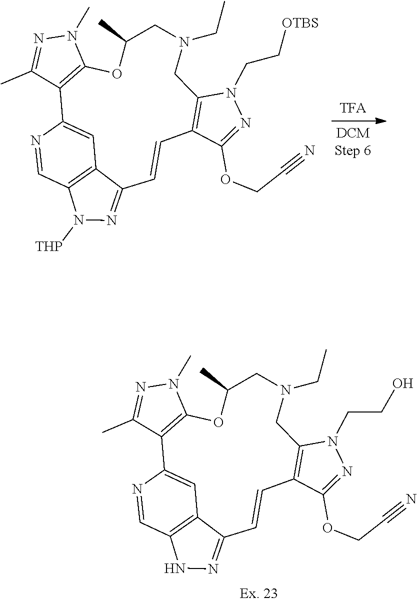

Step 1. To a solution of commercially available (2R)-1-(ethylamino)propan-2-ol (8.13 g, 78.8 mmol, 1 eq) in DCM (80 mL) was added commercially available (2,2,2-trifluoroacetyl) 2,2,2-trifluoroacetate (24.8 g, 118 mmol, 16.4 mL, 1.5 eq) and TEA (39.9 g, 394 mmol, 5 eq) at 0° C., then the mixture was stirred at 0° C. for 1 h. On completion, the reaction mixture was partitioned between DCM (100 mL×3) and water (100 mL). The combined organic phase was dried over anhydrous sodium sulfate, filtered and concentrated to give a residue. The residue was purified by column chromatography (SiO₂, PE:THF=1:1 to 1:1) to give N-ethyl-2,2,2-trifluoro-N-[(2R)-2-hydroxypropyl]acetamide (7.92 g, 39.8 mmol, 50% yield) as a white solid. LCMS: (M+1: 200.1).

Step 2. To a solution of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (1.00 g, 2.95 mmol, 1 eq) in THE (15 mL) was added N-ethyl-2,2,2-trifluoro-N-[(2R)-2-hydroxypropyl]acetamide from step 1 (587 mg, 2.95 mmol, 1 eq), PPh₃ (1.55 g, 5.89 mmol, 2 eq) and DBAD (1.02 g, 4.42 mmol, 1.5 eq). The mixture was stirred at 25° C. for 2 h. On completion, the mixture was concentrated to give a residue. The residue was purified by column chromatography (SiO₂, PE:THF=1:1 to 1:1) to give N-[(2S)-2-[2,5-dimethyl-4-(1-tetrahydropyran-2-yl-3-vinyl-pyrazolol[3,4-c]pyridin-5-yl)pyrazol-3-yl]oxypropyl]-N-ethyl-2,2,2-trifluoro-acetamide (2.3 g, crude) as a white solid. LCMS: (M+1: 521.2).

Step 3. To a solution of N-[(2S)-2-[2,5-dimethyl-4-(1-tetrahydropyran-2-yl-3-vinyl-pyrazolo [3,4-c]pyridin-5-yl)pyrazol-3-yl]oxypropyl]-N-ethyl-2,2,2-trifluoro-acetamide (2.30 g, 4.42 mmol, 1 eq) in THE (23 mL) was added LiOH·H₂O (927 mg, 22.1 mmol, 5 eq) and H₂O (4 mL). The mixture was stirred at 25° C. for 2 h. On completion, the reaction mixture was partitioned between 2-Me THE (25 mL×3) and water (25 mL). The combined organic phase was dried over anhydrous sodium sulfate, filtered and concentrated to give a residue. The residue was purified by column chromatography (SiO₂, DCM:MeOH=5:1 to 5:1) to give (2S)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (566 mg, 1.33 mmol, 30% yield) as a white solid. LCMS: (M+1: 425.2).

Step 4. To a solution of (2S)-2-12,5-dimethyl-4-(1-tetrahydropyran-2-yl-3-vinyl-pyrazolo[3,4-c]pyridin-5-yl)pyrazol-3-yl]oxy-N-ethyl-propan-1-amine (437 mg, 1.03 mmol, 1 eq) in ACN (5 mL) was added 2-[5-(bromomethyl)-1-[2-[tert-butyl(dimethyl)silyl]oxyethyl]-4-iodo-pyrazol-3-yl]oxyacetonitrile, which was prepared according to the method described in Ex. 22, (567 mg, 1.13 mmol, 1.1 eq) and K₂CO₃ (427 mg, 3.09 mmol, 3 eq). The mixture was stirred at 60° C. for 1 h. On completion, the mixture was filtered and concentrated to give a residue. The residue was purified by column chromatography (SiO₂, PE:THF=3:1 to 3:1) to give 2-[1-[2-[tert-butyl(dimethyl) silyl]oxyethyl]-5-[[[(2S)-2-[2,5-dimethyl-4-(1-tetrahydropyran-2-yl-3-vinyl-pyrazolo [3,4-c]pyridin-5-yl)pyrazol-3-yl]oxypropyl]-ethyl-amino]methyl]-4-iodo-pyrazol-3-yl]oxyacetonitrile (800 mg, 948 μmol, 92% yield) as a white solid. LCMS: (M+1: 844.3).

Step 5. The mixture of 2-[1-[2-[tert-butyl(dimethyl)silyl]oxyethyl]-5-[[[(2S)-2-[2,5-dimethyl-4-(1-tetrahydropyran-2-yl-3-vinyl-pyrazolo[3,4-c]pyridin-5-yl)pyrazol-3-yl]oxypropyl]-ethyl-amino]methyl]-4-iodo-pyrazol-3-yl]oxyacetonitrile (400 mg, 474 mol, 1 eq) in DMF (20 mL) was added NaHCO₃ (99.5 mg, 1.19 mmol, 2.5 eq) and Pd(OAc)₂ (21.3 mg, 94.8 μmol, 0.2 eq) and TBAC (132 mg, 474 μmol, 1 eq) degassed and purged with N₂ for 3 times, and then the mixture was stirred at 130° C. for 0.5 h under N₂ atmosphere. On completion, the reaction mixture was partitioned between EA (60 mL×3) and water (60 mL). The combined organic phase was dried over anhydrous sodium sulfate, filtered, and concentrated to give a residue. The residue was purified by column chromatography (SiO₂, PE:THF=1:1 to 1:1) to give {[(10S,17E)-14-(2-{[tert-butyl(dimethyl)silyl]oxy}ethyl)-12-ethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (339 mg, 473 μmol, 99% yield) as a white solid. LCMS: (M+1: 716.3).

Step 6. To a solution of {[(10S,17E)-14-(2-{[tert-butyl(dimethyl)silyl]oxy}ethyl)-12-ethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,41oxazacyclopentadecin-16-yl]oxy}acetonitrile (300 mg, 419 μmol, 1 eq) in DCM (3 mL) was added TFA (4.47 g, 39.2 mmol, 93 eq). The mixture was stirred at 25° C. for 1 h. On completion, the mixture was concentrated to give a residue. The residue was purified by column chromatography (SiO₂, DCM:MeOH=10:1 to 10:1) to give Ex. 23 (90.8 mg, 175 mol, 42% yield) as an orange solid. LCMS: (M+1: 518.2). ¹H NMR for Ex. 23 is shown in the below NMR table.

### Preparation of (2S)-2-[(10S,17E)-16-ethoxy-12-ethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 26)

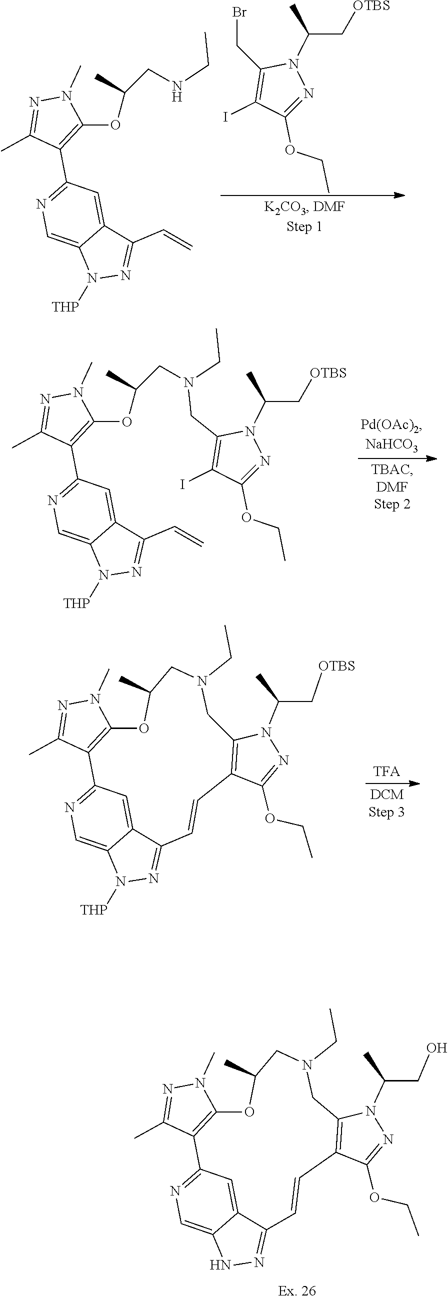

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine, which was prepared according to the method described in Ex. 23, (250 mg, 589 mol, 1 eq) in DMF (3 mL) was added K₂CO₃ (244 mg, 1.77 mmol, 3 eq) and [(2S)-2-[5-(bromomethyl)-3-ethoxy-4-iodo-pyrazol-1-yl]propoxy]-tert-butyl-dimethyl-silane, which was prepared according to the method described in Ex. 19, (385 mg, 766 μmol, 1.3 eq). The mixture was stirred at 80° C. for 2 hr. On completion, the residue was diluted with H₂O (10 mL) and extracted with EA (10 mL×3). The combined organic layers were dried over Na₂SO₄, filtered, and concentrated under reduced pressure to give a residue. The residue was purified by column chromatography (SiO₂, Petroleum ether/Ethyl acetate=1/0 to 0/1) to give (2S)-N-[[2-[(1S)-2-[tert-butyl(dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo [3,4-c]pyridin-5-yl)pyrazol-3-yl]oxy-N-ethyl-propan-1-amine (280 mg, 331 μmol, 56% yield) as yellow oil. LCMS: (M+1=847.2).

Step 2. A mixture of (2S)-N-[[2-[(1S)-2-[tert-butyl (dimethyl)silyl]oxy-1-methyl-ethyl]-5-ethoxy-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (250 mg, 295 μmol, 1 eq), NaHCO₃ (62.0 mg, 738 μmol, 2.5 eq), Pd(OAc)₂ (13.3 mg, 59.0 mol, 0.2 eq) and TBAC (82.0 mg, 295 μmol, 1 eq) in DMF (3 mL) was degassed and purged with N₂ for 3 times, and then the mixture was stirred at 140° C. for 1 h under N₂ atmosphere. On completion, the residue was diluted with H₂O (30 mL) and extracted with EA (15 mL×3). The combined organic layers were dried over Na₂SO₄, filtered, and concentrated under reduced pressure to give a residue. The residue was purified by column chromatography (SiO₂, Petroleum ether/Ethyl acetate=l/0 to 0/1) to give (10S,17E)-14-[(2S)-1-{[tert-butyl(dimethyl)silyl]oxy}propan-2-yl]-16-ethoxy-12-ethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (127 mg, 177 mol, 60% yield) as yellow oil. LCMS: (M+1=719.3).

Step 3. To a solution of (10S,17E)-14-[(2S)-1-{[tert-butyl(dimethyl)silyl]oxy}propan-2-yl]-16-ethoxy-12-ethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (114 mg, 159 μmol, 1 eq) in DCM (0.2 mL) was added TFA (2.76 g, 24.2 mmol, 1.80 mL, 152 eq). The mixture was stirred at 25° C. for 2 hr. On completion, the reaction mixture was concentrated under reduced pressure to remove solvent. The residue was purified by Prep-HPLC (column: Phenomenex luna C18 150*25 mm*10 um; mobile phase: [water(FA)-ACN]; gradient:10%-40% B over 10 min) to give Ex. 26 (50.83 mg, 96.66 μmol, 60.96% yield, 99% purity) as yellow solid. LCMS: (M+1=521.1). ¹H NMR for Ex. 26 is shown in the below NMR table.

### Preparation of 2-[(10S,17E)-16-ethoxy-12-ethyl-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 28)

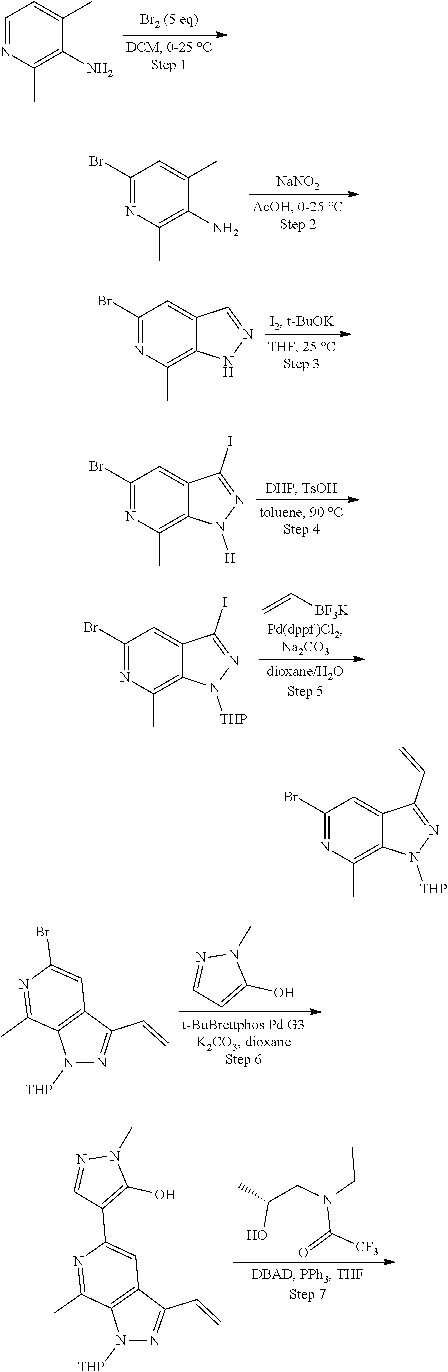

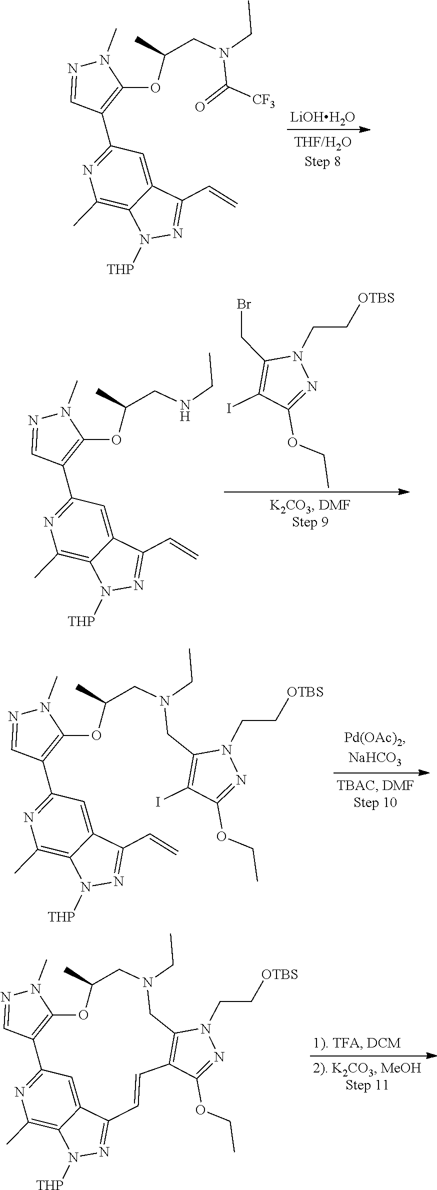

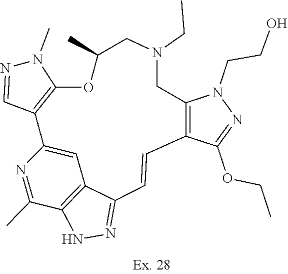 [SMILES: CCOc1nn(CCO)c2c1/C=C/c1n[nH]c3c(C)nc(cc13)-c1cnn(C)c1O[C@@H](C)CN(CC)C2]

Step 1. To a mixture of commercially available 2,4-dimethylpyridin-3-amine (25.0 g, 204.64 mmol, 1 eq) in DCM (500 mL) was added a solution of Br₂ (163 g, 1.02 mol, 52.75 mL, 5 eq) in DCM (300 mL) dropwise at 0° C. The mixture was stirred at 25° C. for 12 hours. To the mixture was added aq. Na₂SO₃ (300 mL) and the mixture was stirred at 25° C. for 1 h. Then the mixture was adjusted to pH=7 by addition of NaHCO₃ slowly. The mixture was extracted with DCM (300 mL×3). The combined organic phase was washed with brine (50 mL), dried with Na₂SO₄, filtered and concentrated in vacuum. The residue was purified by column chromatography (SiO₂, Petroleum ether:Ethyl acetate=1:1) to afford 6-bromo-2,4-dimethyl-pyridin-3-amine (62.0 g, 308 mmol, 75.3% yield, 100% purity) as a yellow oil. ¹H NMR (400 MHz, CDCl₃) δ=7.02 (s, 1H), 3.45 (d, J=16.4 Hz, 2H), 2.37 (s, 3H), 2.13 (s, 3H).

Step 2. To a mixture of 6-bromo-2,4-dimethyl-pyridin-3-amine (30.0 g, 149 mmol, 1 eq) in AcOH (300 mL) was added NaNO₂ (11.3 g, 164 mmol, 1.1 eq) at 0° C. The mixture was stirred at 25° C. for 12 hours. The reaction mixture was concentrated in vacuum. The residue was diluted with H₂O (50 mL) and extracted with DCM (50 mL×3). The combined organic layers were washed with brine (30 mL×3), dried over Na₂SO₄, filtered and concentrated under reduced pressure to give a residue. The crude product was purified by reversed-phase HPLC (0.1% FA in mobile phase) to afford Compound 5-bromo-7-methyl-1H-pyrazolo[3,4-c]pyridine (20.0 g, 94.3 mmol, 63.2% yield) as a yellow solid. ¹H NMR (400 MHz, CDCl₃) δ=11.63-11.02 (m, 1H), 8.09 (s, 1H), 7.71 (s, 1H), 2.84 (s, 3H).

Step 3. To a solution of 5-bromo-7-methyl-1H-pyrazolo[3,4-c]pyridine (34.0 g, 160 mmol, 1 eq) in THE (450 mL) was added t-BuOK (54.0 g, 481 mmol, 3 eq), and was added I₂ (40.7 g, 160 mmol, 32.3 mL, 1 eq). The mixture was stirred at 25° C. for 3 hr. On completion, filtered and the filtrate was concentrated under reduced pressure to give a residue. The residue was purified by CombiFlash (12 g silica gel column, THE in PE 0-100%) to give 5-bromo-3-iodo-7-methyl-1H-pyrazolo [3, 4-c]pyridine (51.0 g, 151 mmol, 94.1% yield) as yellow solid. LCMS: (M+1:337.9).

Step 4. To a solution of 5-bromo-3-iodo-7-methyl-1H-pyrazolo[3,4-c]pyridine (3.10 g, 9.17 mmol, 1 eq) in toluene (31 mL) was added TsOH (316 mg, 1.83 mmol, 0.2 eq) and 3,4-dihydro-2H-pyran (1.93 g, 22.9 mmol, 2.10 mL, 2.5 eq). The mixture was stirred at 90° C. for 16 hr, filtered and the filtrate was concentrated in vacuum. The residue was purified by CombiFlash (12 g silica gel column, EtOAc in PE 0~100%). 5-bromo-3-iodo-7-methyl-1-tetrahydropyran-2-yl-pyrazolo [3, 4-c]pyridine (2.57 g, 6.09 mmol, 66.38% yield) was obtained as light yellow solid. ¹H NMR (400 MHz, DMSO-d₆) S=7.53 (s, 1H), 6.12 (dd, J=9.2, 2.2 Hz, 1H), 3.88 (d, J=11.6 Hz, 1H), 3.73 (d, J=2.4 Hz, 1H), 2.90 (s, 3H), 2.45-2.37 (m, 1H), 2.12-2.03 (m, 2H), 1.81-1.66 (m, 2H), 1.47-1.43 (m, 1H). LCMS: (M+1:421.9).

Step 5. To a solution of potassium vinyltrifluoroborate (816 mg, 6.09 mmol, 1 eq), 5-bromo-3-iodo-7-methyl-1-tetrahydropyran-2-yl-pyrazolo[3,4-c]pyridine (2.57 g, 6.09 mmol, 1 eq) in a mixture solvent of dioxane (25 mL) and H₂O (5 ml) was added Pd(dppf)Cl₂ (445 mg, 609 umol, 0.1 eq) and Na₂CO3 (1.94 g, 18.3 mmol, 3 eq). The mixture was stirred at 80° C. for 16 hr under N₂. Dried with anhydrous Na₂SO₄, filtered and concentrated in vacuum. The residue was purified by CombiFlash (20 silica gel column, DCM 100%) to give 5-bromo-7-methyl-1-tetrahydropyran-2-yl-3-vinyl-pyrazolo [3, 4-c]pyridine (1.00 g, 3.10 mmol, 50.9% yield) as black, brown solid. LCMS: (M+1: 322.0).

Step 6. To a solution of 5-bromo-7-methyl-1-tetrahydropyran-2-yl-3-vinyl-pyrazolo[3,4-c]pyridine (3.50 g, 10.8 mmol, 1 eq), commercially available 2-methylpyrazol-3-ol (1.17 g, 12.0 mmol, 1.1 eq), K₂CO₃ (4.50 g, 32.6 mmol, 3 eq) in dioxane (50 mL) was added [2-(2-aminophenyl)phenyl]-methylsulfonyloxy-palladium; ditert-butyl-[3,6-dimethoxy-2-(2,4,6-triisopropylphenyl)phenyl]phosphane (1.39 g, 1.63 mmol, 0.15 eq). The mixture was stirred at 110° C. for 1 h. On completion, the resulting mixture was quenched with H₂O (200 mL) and extracted with EtOAc (3×200 mL). The combined organic layers were washed with brine (3×200 mL), dried over Na₂SO₄ and concentrated in vacuum. The residue was purified by column chromatography (SiO₂, DCM/MeOH=1/0 to 10/1) to give 2-methyl-4-(7-methyl-1-tetrahydropyran-2-yl-3-vinyl-pyrazolo[3,4-c]pyridin-5-yl)pyrazol-3-ol (3.50 g, 7.22 mmol, 66% yield, 70% purity) as yellow oil. ¹H NMR (400 MHz, CDCl₃) δ 7.72 (s, 1H), 7.61 (s, 1H), 7.00 (dd, J=11.2, 18.0 Hz, 1H), 6.12 (d, J=18.0 Hz, 1H), 5.84 (dd, J=2.0, 9.2 Hz, 1H), 5.60 (d, J=11.2 Hz, 1H), 4.07-3.97 (m, 1H), 3.73 (s, 3H), 2.99 (s, 3H), 2.74-2.63 (m, 1H), 2.26-2.15 (m, 2H), 1.80-1.63 (m, 4H). LCMS: (M+1: 340.0).

Step 7. A solution of 2-methyl-4-(7-methyl-1-tetrahydropyran-2-yl-3-vinyl-pyrazolo[3,4-c]pyridin-5-yl)pyrazol-3-ol (1.85 g, 5.45 mmol, 1 eq), N-ethyl-2,2,2-trifluoro-N-[(2R)-2-hydroxypropyl]acetamide, which was prepared according to the method described in Ex. 23, (1.19 g, 6.00 mmol, 1.1 eq), PPh₃ (3.15 g, 11.9 mmol, 2.2 eq), DBAD (2.76 g, 11.9 mmol, 2.2 eq) in THE (20 mL) was stirred at 25° C. for 1 h. On completion, the mixture was concentrated in vacuum. The residue was purified by column chromatography (SiO₂, Petroleum ether/THF=1/0 to 5/1) to give N-ethyl-2,2,2-trifluoro-N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]acetamide (2.61 g, 2.26 mmol, 41% yield, 45% purity) as a yellow solid. LCMS: (M+1: 521.3).

Step 8. A solution of N-ethyl-2,2,2-trifluoro-N-[(2S)-2-[2-methyl-4-(7-methyl-1-tetrahydropyran-2-yl-3-vinyl-pyrazolo[3,4-c]pyridin-5-yl)pyrazol-3-yl]oxypropyl]acetamide (2.41 g, 4.63 mmol, 1 eq), LiOH·H₂O (1.55 g, 37.0 mmol, 8 eq) in THE (30 mL) and H₂O (6 mL) was stirred at 25° C. for 1 h. To the mixture was added water (20 mL) and extracted with EA (3×50 mL). The combined organic layers were washed with brine (2×50 mL), dried over Na₂SO₄, filtered and concentrated in vacuum to give (2S)-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (2.13 g, 2.09 mmol, 45% yield, 41% purity) as a yellow solid. LCMS: (M+1: 425.3).

Step 9. A solution of (2S)-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (2.00 g, 1.93 mmol, 1 eq), 2-[5-(bromomethyl)-3-ethoxy-4-iodo-pyrazol-1-yl]ethoxy-tert-butyl-dimethyl-silane, prepared according to the method described in Ex. 1, (945 mg, 1.93 mmol, 1 eq), K₂CO₃ (800 mg, 5.79 mmol, 3 eq) in DMF (20 mL) was stirred at 80° C. for 1 h. On completion, the mixture was concentrated in vacuum. The residue was purified by column chromatography (SiO₂, Petroleum ether/THF=1/0 to 5/1) to give (2S)-N-[[2-[2-[tert-butyl(dimethyl)silyl]oxyethyl]-5-ethoxy-4-iodo-pyrazol-3-yl]methyl]-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (1.20 g, 1.11 mmol, 57% yield, 77% purity) as a yellow solid. ¹H NMR (400 MHz, CDCl₃) δ 7.82 (d, J=3.2 Hz, 1H), 7.79 (d, J=1.2 Hz, 1H), 7.08-6.94 (m, 1H), 6.10 (d, J=18.0 Hz, 1H), 5.89 (dd, J=2.0, 9.6 Hz, 1H), 5.58 (d, J=11.2 Hz, 1H), 4.48-4.37 (m, 1H), 4.22 (dq, J=4.8, 7.2 Hz, 3H), 4.15-4.01 (m, 2H), 3.86-3.76 (m, 3H), 3.69 (d, J=3.6 Hz, 3H), 3.65-3.51 (m, 2H), 3.00 (s, 3H), 2.79-2.68 (m, 2H), 2.60-2.42 (m, 3H), 2.26-2.13 (m, 2H), 1.77 (t, J=9.2 Hz, 2H), 1.65 (s, 1H), 1.39 (dt, J=2.4, 7.2 Hz, 3H), 1.12 (dd, J=6.4, 8.4 Hz, 3H), 0.97 (dt, J=5.2, 7.2 Hz, 3H), 0.77 (d, J=1.2 Hz, 9H), −0.10-0.15 (m, 6H). LCMS: (M+1: 833.9).

Step 10. To a solution of (2S)-N-|[2-|2-|tert-butyl(dimethyl)silyl|oxyethyl|-5-ethoxy-4-iodo-pyrazol-3-yl]methyl]-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (1.17 g, 1.40 mmol, 1 eq), was added Pd(OAc)₂ (63.1 mg, 280 mol, 0.2 eq), TBAC (390 mg, 1.40 mmol, 1 eq), NaHCO₃ (295 mg, 3.51 mmol, 2.5 eq) in DMF (30 mL). The mixture was stirred at 120° C. for 16 h. On completion, the mixture was concentrated in vacuum. The residue was purified by column chromatography (SiO₂, Petroleum ether/THF=1/0 to 2/1) to give (10S,17E)-14-(2-{[tert-butyl(dimethyl)silyl]oxy}ethyl)-16-ethoxy-12-ethyl-8,10,20-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (440 mg, 536 μmol, 38% yield, 86% purity) as yellow solid. LCMS: (M+1: 705.7).

Step 11. To a solution of (10S,17E)-14-(2-{[tert-butyl(dimethyl)silyl]oxy}ethyl)-16-ethoxy-12-ethyl-8,10,20-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (380 mg, 539 μmol, 1 eq) in DCM (3 mL), then was added TFA (4.61 g, 40.3 mmol, 3 mL, 74.9 eq). The mixture was stirred at 25° C. for 0.5 h. On completion, the mixture was concentrated in vacuum to give a yellow oil (324 mg, 538 mol, 99% yield). LCMS: (M+1: 603.2).

Then, 300 mg of the yellow oil obtained above was dissolved in MeOH (5 mL), and K₂CO₃ (688 mg, 4.98 mmol, 10 eq) was added. The mixture was stirred at 25° C. for 10 min. On completion, the mixture was filtered and concentrated in vacuum. The residue was purified by Prep-HPLC (column: Welch Ultimate C18 150*25 mm*5 um; mobile phase: [water(FA)-ACN]; gradient:7%-37% B over 10 min) to give Ex. 28 (65.09 mg, 0.118 mmol, 23.7% yield, FA) was obtained as a yellow solid. LCMS: (M+1: 507.3). ¹H NMR for Ex. 28 is shown in the below NMR table.

### Preparation of 2-[(10S,17E)-16-ethoxy-12-ethyl-8,10-dimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 34)

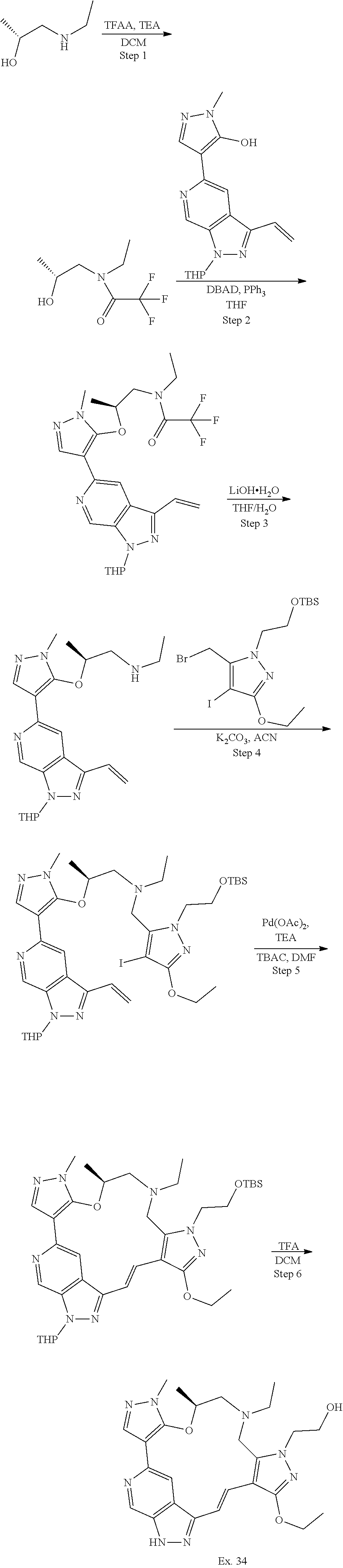

Step 1. To a solution of commercially available (R)-1-(ethylamino)propan-2-ol (1.00 g, 9.69 mmol, 1 eq) in DCM (10 mL) was added 2,2,2-trifluoroacetic anhydride (3.05 g, 14.5 mmol, 1.5 eq) and TEA (4.90 g, 48.5 mmol, 7 mL, 5 eq) at 0° C., then the mixture was stirred at 0° C. for 1 h. On completion, the reaction mixture was diluted with H₂O (20 mL) and extracted with DCM (15 mL×3). The combined organic layers were dried over Na₂SO₄, filtered, and concentrated under reduced pressure. The residue was purified by flash silica gel chromatography (ISCO®; 40 g SepaFlash® Silica Flash Column, Eluent of 0-50% Ethyl acetate/Petroleum ether gradient at 40 mL/min). Compound (R)-N-ethyl-2,2,2-trifluoro-N-(2-hydroxypropyl)acetamide (1.30 g, 67% yield) was obtained as a yellow oil. LCMS: (M+1:200.0).

Step 2. The mixture of (R)-N-ethyl-2,2,2-trifluoro-N-(2-hydroxypropyl)acetamide (1.30 g, 6.53 mmol, 1 eq), 2-methyl-4-(1-tetrahydropyran-2-yl-3-vinyl-pyrazolo[3,4-c]pyridin-5-yl)pyrazol-3-ol, which was prepared according to the method described in Ex. 5, (2.12 g, 6.53 mmol, 1 eq), DBAD (2.25 g, 9.79 mmol, 1.5 eq) and PPh₃ (3.42 g, 13.1 mmol, 2 eq) in THE (30 mL) under N₂ was stirred at 25° C. for 2 hr. On completion, the reaction mixture was concentrated under reduced pressure. The residue was purified by flash silica gel chromatography (ISCO®; 80 g SepaFlash® Silica Flash Column, Eluent of 0-100% THE/Petroleum ether gradient at 80 mL/min). Compound N-ethyl-2,2,2-trifluoro-N-((2S)-2-((1-methyl-4-(1-(tetrahydro-2H-pyran-2-yl)-3-vinyl-1H-pyrazolo[3,4-c]pyridin-5-yl)-1H-pyrazol-5-yl)oxy)propyl)acetamide (4.80 g, crude) was obtained as a yellow solid. LCMS (M+1:507.1).

Step 3. To a solution of N-ethyl-2,2,2-trifluoro-N-((2S)-2-((1-methyl-4-(1-(tetrahydro-2H-pyran-2-yl)-3-vinyl-1H-pyrazolo[3,4-c]pyridin-5-yl)-1H-pyrazol-5-yl)oxy)propyl)acetamide (4.70 g, 9.28 mmol, 1 eq) in THE (50 mL) and H₂O (10 mL) was added LiOH·H₂O (1.95 g, 46.4 mmol, 5 eq), then the mixture was stirred at 25° C. for 1 h. On completion, the reaction mixture was diluted with H₂O (60 mL) and extracted with EA (30 mL*3). The combined organic layers were washed with Brine (30 mL*2), dried over Na₂SO₄, filtered and concentrated under reduced pressure to give a residue. The residue was purified by flash silica gel chromatography (ISCO®; 80 g SepaFlash® Silica Flash Column, Eluent of 0-15% MeOH/DCMradient at 60 mL/min) to give compound (2S)-N-ethyl-2-((1-methyl-4-(1-(tetrahydro-2H-pyran-2-yl)-3-vinyl-1H-pyrazolo[3,4-c]pyridin-5-yl)-1H-pyrazol-5-yl)oxy)propan-1-amine (1.40 g, 3.41 mmol, 37% yield) as a yellow oil. LCMS (M+1:411.1).

¹H NMR (400 MHz, DMSO-d₆) δ=9.19 (s, 1H), 8.13 (s, 1H), 7.92 (d, J=1.6 Hz, 1H), 7.12-6.98 (m, 1H), 6.21 (d, J=18.0 Hz, 1H), 5.97 (br d, J=9.6 Hz, 1H), 5.60 (d, J=11.6 Hz, 1H), 4.49 (br s, 1H), 3.90 (br d, J=11.6 Hz, 1H), 3.83-3.73 (m, 1H), 3.70 (s, 3H), 3.59 (br t, J=6.4 Hz, 1H), 2.89-2.71 (m, 2H), 2.63-2.54 (m, 2H), 2.44-2.30 (m, 1H), 2.02 (br d, J=10.4 Hz, 2H), 1.78-1.72 (m, 1H), 1.60 (br s, 2H), 1.19 (d, J=6.4 Hz, 3H), 1.05-0.96 (m, 3H)

Step 4. To a solution of (2S)-N-ethyl-2-[2-methyl-4-(1-tetrahydropyran-2-yl-3-vinyl-pyrazolo[3,4-c]pyridin-5-yl)pyrazol-3-yl]oxy-propan-1-amine (200 mg, 487 mol, 1 eq) in ACN (5 mL) was added 2-[5-(bromomethyl)-3-ethoxy-4-iodo-pyrazol-1-yl]ethoxy-tert-butyl-dimethyl-silane, prepared according to the method described in Ex. 1, (286 mg, 585 mol, 1.2 eq) and K₂CO₃ (202 mg, 1.46 mmol, 3 eq), then the mixture was stirred at 60° C. for 1 h. On completion, the reaction mixture was concentrated under reduced pressure to give a residue. The residue was purified by flash silica gel chromatography (ISCO®; 12 g SepaFlash® Silica Flash Column, Eluent of 0-50% Ethyl acetate/Petroleum ether gradient at 40 mL/min). Compound (2S)-N-[[2-[2-[tert-butyl(dimethyl)silyl]oxyethyl]-5-ethoxy-4-iodo-pyrazol-3-yl]methyl]-N-ethyl-2-[2-methyl-4-(1-tetrahydropyran-2-yl-3-vinyl-pyrazolo[3,4-c]pyridin-5-yl)pyrazol-3-yl]oxy-propan-1-amine (340 mg, 415 mol, 85% yield) was obtained as a yellow oil. LCMS: (M+1:819.2).

Step 5. To a solution of (2S)-N-[[2-[2-[tert-butyl(dimethyl)silyl]oxyethyl]-5-ethoxy-4-iodo-pyrazol-3-yl]methyl]-N-ethyl-2-[2-methyl-4-(1-tetrahydropyran-2-yl-3-vinyl-pyrazolo [3,4-c]pyridin-5-yl)pyrazol-3-yl]oxy-propan-1-amine (300 mg, 366 μmol, 1 eq) in DMF (5 mL) was added Pd(OAc)₂ (16.5 mg, 73 mol, 0.2 eq), TBAC (102 mg, 366 μmol, 1 eq) and TEA (148 mg, 1.47 mmol, 4 eq) under N₂, then the mixture was stirred at 130° C. for 1 h. On completion, the reaction mixture was diluted with 10 mL H₂O and extracted with 15 mL EA (5 mL×3). The combined organic layers were washed with 10 mL Brine (5 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0-50% THF/Petroleum ether gradient at 40 mL/min). Compound (10S,17E)-14-(2-{[tert-butyl(dimethyl)silyl]oxy}ethyl)-16-ethoxy-12-ethyl-8,10-dimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (100 mg, 145 mol, 40% yield) was obtained as a yellow oil. LCMS: (M+1:691.3).

Step 6. To a solution of (10S,17E)-14-(2-{[tert-butyl(dimethyl)silyl]oxy}ethyl)-16-ethoxy-12-ethyl-8,10-dimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (80 mg, 116 μmol, 1 eq) in DCM (1 mL) was added TFA (1.23 g, 10.7 mmol, 93 eq), then the mixture was stirred at 25° C. for 1 h. On completion, the reaction mixture was concentrated under reduced pressure to give a residue. The residue was purified by prep-HPLC (column: Welch Xtimate C18 150*25 mm*5 um; mobile phase: [water(TFA)-ACN]; gradient:8%-38% B over 3 min). Compound Ex. 34 (12.45 mg, 25.28 μmol, 21.83% yield) was obtained as a yellow solid. LCMS: (M+1:493.1). ¹H NMR for Ex. 34 is shown in the below NMR table.

### Preparation of {[(10R,17E)-12-ethyl-14-(2-hydroxyethyl)-6,8,10-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 36)

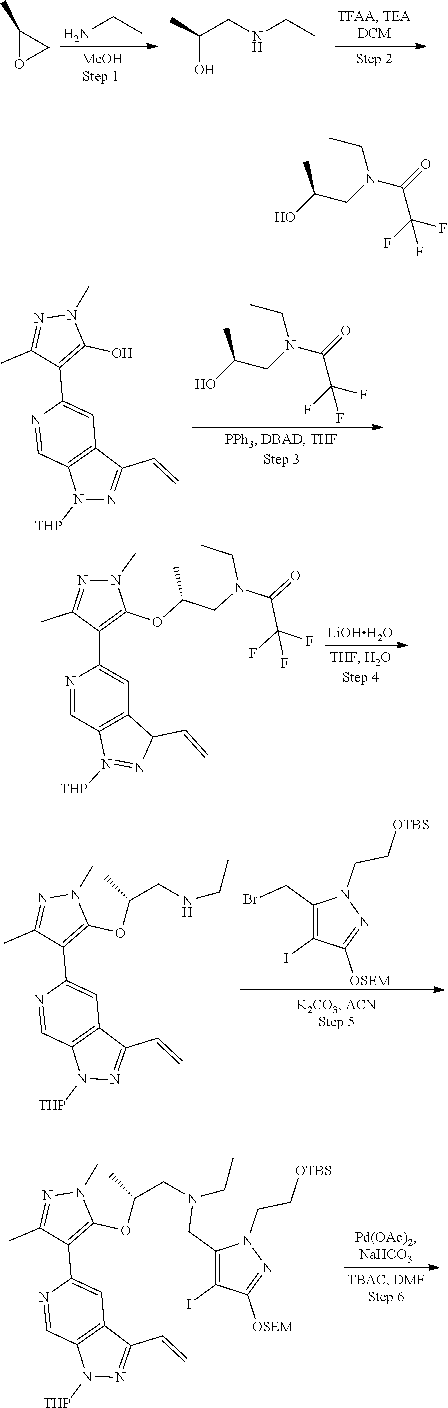

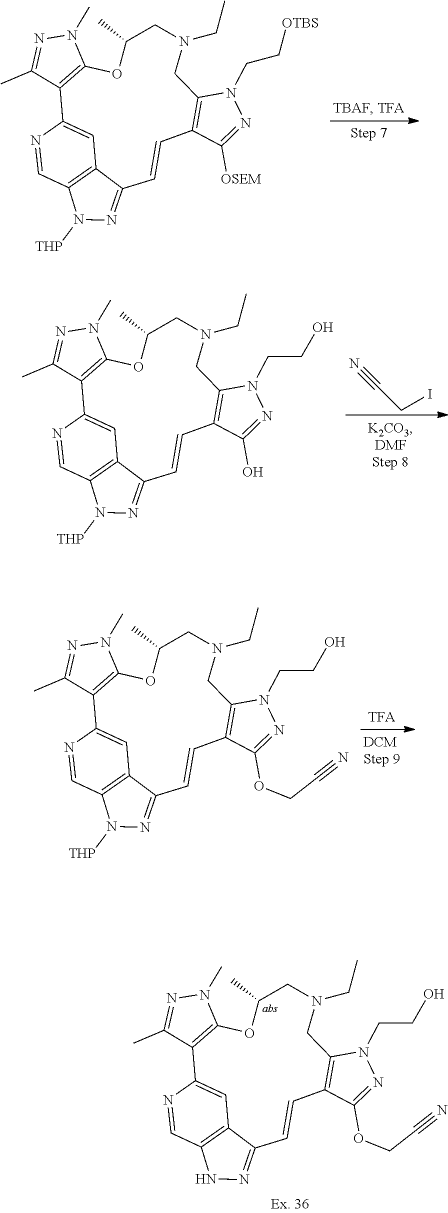

Step 1. To a solution of commercially available (2S)-2-methyloxirane (10.0 g, 172 mmol, 1 eq) in MeOH (100 mL) was added commercially available ethanamine (2 M, 258 mL, 3 eq). The mixture was stirred at 25° C. for 16 h. On completion, the reaction mixture was concentrated under reduced pressure to give (2S)-1-(ethylamino) propan-2-ol (6.50 g, crude) as a colorless liquid. ¹H NMR (400 MHz, DMSO-d₆) δ=3.69-3.56 (m, 1H), 2.55-2.44 (m, 2H), 2.42-2.32 (m, 2H), 1.11-0.85 (m, 6H).

Step 2. To a solution of (2S)-1-(ethylamino)propan-2-ol (5.70 g, 55.2 mmol, 1 eq) in DCM (57 mL) was added commercially available (2,2,2-trifluoroacetyl) 2,2,2-trifluoroacetic anhydride (17.4 g, 82.8 mmol, 1.5 eq) and TEA (27.9 g, 276 mmol, 5 eq) at 0° C. The mixture was stirred at 0° C. for 1 h. On completion, the reaction mixture was partitioned between DCM (60 mL×3) and water (60 mL). The combined organic phase was dried over anhydrous sodium sulfate, filtered, and concentrated to give a residue. The residue was purified by column chromatography (SiO₂, PE:THF=2:1 to 2:1) to give N-ethyl-2,2,2-trifluoro-N-[(2S)-2-hydroxypropyl]acetamide (6.41 g, 32.2 mmol, 58% yield) as a white solid. LCMS: (M+1: 200.1).

Step 3. To a solution of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (600 mg, 1.77 mmol, 1 eq) in THE (9 mL) was added N-ethyl-2,2,2-trifluoro-N-[(2S)-2-hydroxypropyl]acetamide from step 2 (352 mg, 1.77 mmol, 1 eq), PPh₃ (927 mg, 3.54 mmol, 2 eq) and DBAD (611 mg, 2.65 mmol, 1.5 eq) under N₂. The mixture was stirred at 25° C. for 2 h. On completion, the mixture was concentrated to give a residue. The residue was purified by column chromatography (SiO₂, PE:THF=1:1 to 1:1) to give N-[(2R)-2-[2,5-dimethyl-4-(1-tetrahydropyran-2-yl-3-vinyl-pyrazolo[3,4-c]pyridin-5-yl)pyrazol-3-yl]oxypropyl]-N-ethyl-2,2,2-trifluoro-acetamide (1.53 g, crude) as a white solid. LCMS: (M+1: 521.0).

Step 4. To a solution of N-[(2R)-2-[2,5-dimethyl-4-(1-tetrahydropyran-2-yl-3-vinyl-pyrazolo [3,4-c]pyridin-5-yl)pyrazol-3-yl]oxypropyl]-N-ethyl-2,2,2-trifluoro-acetamide (1.43 g, 2.75 mmol, 1 eq) in THE (14.3 mL) was added LiOH·H2O (576 mg, 13.7 mmol, 5 eq) and H₂O (2.86 mL). The mixture was stirred at 25° C. for 2 h. On completion, the reaction mixture was partitioned between 2-MeTHF (20 mL×3) and water (20 mL). The combined organic phase was dried over anhydrous sodium sulfate, filtered and concentrated to give a residue. The residue was purified by column chromatography (SiO₂, DCM:MeOH) to give (2R)-2-12,5-dimethyl-4-(1-tetrahydropyran-2-yl-3-vinyl-pyrazolo[3,4-c]pyridin-5-yl)pyrazol-3-yl]oxy-N-ethyl-propan-1-amine (470 mg, 1.11 mmol, 40% yield) as a white solid. LCMS: (M+1: 425.0).

Step 5. To a solution of (2R)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (430 mg, 1.01 mmol, 1 eq) in ACN (5 mL) was added 2-[[5-(bromomethyl)-1-[2-[tert-butyl(dimethyl)silyl]oxyethyl]-4-iodo-pyrazol-3-yl]oxymethoxy]ethyl-trimethyl-silane, which was prepared according to the method described in Ex. 42, (659 mg, 1.11 mmol, 1.1 eq) and K₂CO₃ (420 mg, 3.04 mmol, 3 eq). The mixture was stirred at 60° C. for 2 h. On completion, the mixture was filtered and concentrated to give a residue. The residue was purified by column chromatography (SiO₂, PE:THF=2:1 to 2:1) to give (2R)-N-[[2-[2-[tert-butyl(dimethyl)silyl]oxyethyl]-4-iodo-5-(2-trimethylsilylethoxymethoxy) pyrazol-3-yl]methyl]-2-[2,5-dimethyl-4-(1-tetrahydropyran-2-yl-3-vinyl-pyrazolo[3, 4-c]pyridin-5-yl)pyrazol-3-yl]oxy-N-ethyl-propan-1-amine (876 mg, 937 mol, 92% yield) as a white solid. LCMS: (M+1: 935.7).

Step 6. To a solution of (2R)-N-[[2-[2-[tert-butyl(dimethyl)silyl]oxyethyl]-4-iodo-5-(2-trimethylsilylethoxymethoxy)pyrazol-3-yl]methyl]-2-[2,5-dimethyl-4-(1-tetrahydropyan-2-yl-3-vinyl-pyrazolo[3,4-c]pyridin-5-yl)pyrazol-3-yl]oxy-N-ethyl-propan-1-amine (796 mg, 851 mol, 1 eq) in DMF (40 mL) was added NaHCO₃ (179 mg, 2.13 mmol, 2.5 eq), TBAC (236 mg, 851 μmol, 1 eq) and Pd(OAc)₂ (38.2 mg, 170 μmol, 0.2 eq). The mixture was degassed and purged with N₂ for 3 times, and then the mixture was stirred at 130° C. for 0.5 h under N₂ atmosphere. On completion, the reaction mixture was partitioned between EA (120 mL×3) and water (120 mL). The combined organic phase was dried over anhydrous sodium sulfate, filtered and concentrated to give a residue. The residue was purified by column chromatography (SiO₂, PE:THF=2:1 to 2:1) to give (10R,17E)-14-(2-{[tert-butyl(dimethyl)silyl]oxy}ethyl)-12-ethyl-6,8,10-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (614 mg, 761 μmol, 89% yield) as a white solid. LCMS: (M+1: 807.3).

Step 7. To a solution of (10R,17E)-14-(2-{[tert-butyl(dimethyl)silyl]oxy}ethyl)-12-ethyl-6,8,10-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (250 mg, 310 mol, 1 eq) in THE (1.86 mL) was added TBAF (1 M, 1.86 mL, 6 eq). The mixture was stirred at 70° C. for 3 h. On completion, the reaction mixture was partitioned between EA (2 mL×3) and water (5 mL). The combined organic phase was dried over anhydrous sodium sulfate, filtered and concentrated to give a residue. The residue was purified by column chromatography (SiO₂, DCM:MeOH=15:1 to 15:1) to give (10R,17E)-12-ethyl-14-(2-hydroxyethyl)-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (133 mg, 236 μmol, 76% yield) as a yellow solid. LCMS: (M+1: 563.2).

Step 8. To a solution of (10R,17E)-12-ethyl-14-(2-hydroxyethyl)-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (118 mg, 210 mol, 1 eq) in DMF (1.5 mL) was added K₂CO₃ (58.0 mg, 419 mol, 2 eq) and commercially available 2-iodoacetonitrile (52.5 mg, 314 μmol, 1.5 eq). The mixture was stirred at 80° C. for 2 h. On completion, the reaction mixture was partitioned between EA (5 mL×3) and water (5 mL). The combined organic phase was dried over anhydrous sodium sulfate, filtered and concentrated to give a residue. The residue was purified by column chromatography (SiO₂, DCM:MeOH=20:1 to 20:1) to give {[(10R,17E)-12-ethyl-14-(2-hydroxyethyl)-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (82.0 mg, 136 mol, 65% yield) as a brown solid. LCMS: (M+1: 602.2).

Step 9. To a solution of {[(10R,17E)-12-ethyl-14-(2-hydroxyethyl)-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (67.0 mg, 111 μmol, 1 eq) in DCM (0.5 mL) was added TFA (685 mg, 6.01 mmol, 54 eq). The mixture was stirred at 25° C. for 1 h. On completion, the mixture was concentrated to give a residue. The residue was purified by reversed phase column (column: Waters xbridge 150*25 mm 10 um; mobile phase: [water(NH₄HCO₃)-ACN]; gradient:22%-52% B over 14 min) to give Ex. 36 (18.9 mg, 36.52 μmol, 32.79% yield) as a yellow solid. LCMS: (M+1: 518.2). ¹H NMR for Ex. 36 is shown in the below NMR table.

### Preparation of 2-[(10R,17E)-16-ethoxy-12-ethyl-8,10,20-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 37)

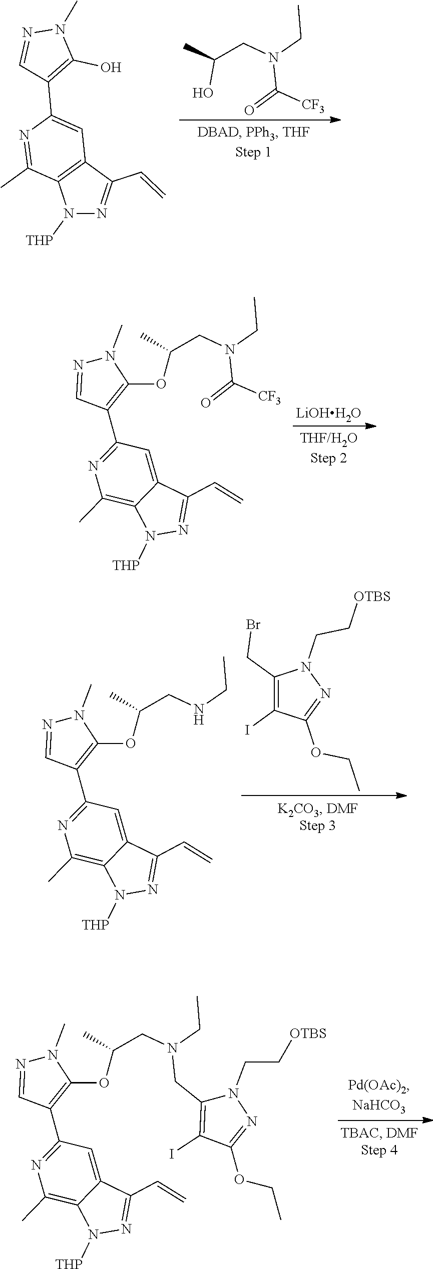

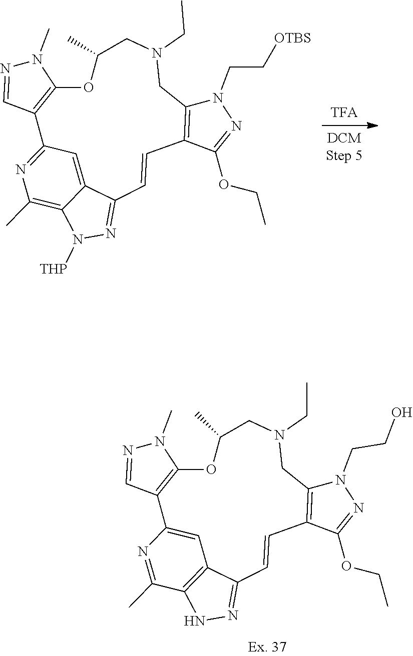

Step 1. The mixture of 2-methyl-4-(7-methyl-1-tetrahydropyran-2-yl-3-vinyl-pyrazolo[3,4-c]pyridin-5-yl)pyrazol-3-ol, which was prepared according to the method described in Ex. 28, (1.60 g, 4.71 mmol, 1 eq), commercially available N-ethyl-2,2,2-trifluoro-N-[(2S)-2-hydroxypropyl]acetamide (1.88 g, 9.43 mmol, 2 eq), PPh₃ (2.47 g, 9.43 mmol, 2 eq) and DBAD (2.71 g, 11.8 mmol, 2.5 eq) was degassed and purged with N₂ for 3 times, then THE (15 mL) was added dropwise to the mixture at 0° C., the resulting mixture was stirred for another 2 h at 25° C. under N₂. On completion, the reaction mixture was concentrated in vacuo. The residue was purified by column chromatography on silica gel (12 g silica gel, EA in Petroleum ether from 0% to 100%) to give N-ethyl-2,2,2-trifluoro-N-[(2R)-2-[2-methyl-4-(7-methyl-1-tetrahydropyran-2-yl-3-vinyl-pyrazolo[3,4-c]pyridin-5-yl)pyrazol-3-yl]oxypropyl]acetamide (3.40 g, 2.01 mmol, 42% yield, 31% purity) as a yellow oil. LCMS: (M+1: 521.1).

Step 2. A solution of N-ethyl-2,2,2-trifluoro-N-[(2R)-2-[2-methyl-4-(7-methyl-1-tetrahydropyran-2-yl-3-vinyl-pyrazolo[3,4-c]pyridin-5-yl)pyrazol-3-yl]oxypropyl]acetamide (3.30 g, 6.34 mmol, 1 eq) and LiOH·H₂O (1.33 g, 31.7 mmol, 5 eq) in THE (30 mL) and H₂O (6 mL) was stirred at 25° C. for 1 h. On completion, the mixture was diluted with water (120 mL) and extracted with ethyl acetate (30 mL×3). The combined organic phase was washed with saturated sodium chloride solution (30 mL×2) and dried over Na₂SO₄, filtered and the filtrate was concentrated to give a residue. The residue was purified by column chromatography on silica gel (12 g silica gel, EA in Petroleum ether from 0% to 100%) to give (2R)-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (1.00 g, 2.21 mmol, 35% yield, 94% purity) as a yellow solid. ¹H NMR (400 MHz, CDCl₃) δ=7.85-7.81 (m, 2H), 7.02 (dd, J=11.6, 18.0 Hz, 1H), 6.13 (d, J=18.0 Hz, 1H), 5.89 (d, J=9.2 Hz, 1H), 5.57 (d, J=11.6 Hz, 1H), 4.56-4.44 (m, 1H), 4.08-4.02 (m, 1H), 3.84-3.71 (m, 5H), 3.03-3.00 (m, 3H), 2.72-2.64 (m, 3H), 2.25-2.10 (m, 3H), 1.64 (s, 4H), 1.22 (t, J=6.8 Hz, 3H), 1.12 (dt, J=4.4, 7.2 Hz, 3H).

Step 3. To a solution of (2R)-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (350 mg, 824 mol, 1 eq), 2-15-(bromomethyl)-3-ethoxy-4-iodo-pyrazol-1-yl]ethoxy-tert-butyl-dimethyl-silane, prepared according to the method described in Ex. 1, (444 mg, 907 mol, 1.1 eq) and K₂CO₃(342 mg, 2.47 mmol, 3 eq) in DMF (5 mL) was stirred at 60° C. for 2 h. On completion, the mixture was diluted with water (30 mL) and extracted with ethyl acetate (20 mL×3). The combined organic phase was washed with saturated sodium chloride solution (20 mL×2) and dried over Na₂SO₄, filtered and the filtrate was concentrated to give a residue. The residue was purified by column chromatography on silica gel (4 g silica gel, EA in Petroleum ether from 0% to 100%) to give (2R)-N-[[2-[2-[tert-butyl(dimethyl)silyl]oxyethyl]-5-ethoxy-4-iodo-pyrazol-3-yl]methyl]-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (770 mg, 793 mol, 96% yield, 86% purity) as a brown solid. LCMS: (M+1: 833.4).

Step 4. A solution of (2R)-N-[[2-[2-[tert-butyl(dimethyl)silyl]oxyethyl]-5-ethoxy-4-iodo-pyrazol-3-yl]methyl]-N-ethyl-2-[2-methyl-4-(7-methyl-1-tetrahydropyran-2-yl-3-vinyl-pyrazolo[3,4-c]pyridin-5-yl)pyrazol-3-yl]oxy-propan-1-amine (700 mg, 840 mol, 1 eq), NaHCO₃ (176 mg, 2.10 mmol, 2.5 eq), TBAC (234 mg, 840 μmol, 1 eq), diacetoxypalladium (37.7 mg, 168 mol, 0.2 eq) in DMF (70 mL) was degassed and purged with N₂ for 3 times, and then the mixture was stirred at 120° C. for 16 h under N₂ atmosphere. On completion, the mixture was poured into H₂O (200 mL) and extracted with EA (70 mL×3). The combined organic phase was washed with H₂O (300 mL×3) and dried over anhydrous sodium sulfate, filtered and concentrated to give a residue. The residue was purified by column chromatography (SiO₂, Petroleum ether/THF=3:1 to 0:1) to give (10R,17E)-14-(2-{[tert-butyl(dimethyl)silyl]oxy}ethyl)-16-ethoxy-12-ethyl-8,10,20-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (380 mg, 458 μmol, 54% yield, 85% purity) as a brown oil. LCMS: (M+1: 705.3).

Step 5. To a solution of (10R,17E)-14-(2-{[tert-butyl(dimethyl)silyl]oxy}ethyl)-16-ethoxy-12-ethyl-8,10,20-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (350 mg, 496 μmol, 1 eq) in DCM (3 mL) was added TFA (8.08 mmol, 0.6 mL, 16.3 eq). The mixture was stirred at 25° C. for 1 h. On completion, the reaction mixture was concentrated in vacuo. The mixture was purified by Prep-HPLC (column: Phenomenex luna C18 150*25 mm*10 um; mobile phase: [water(FA)-ACN]; gradient:7%-37% B over 15 min). The solvent was lyophilized to give Ex. 37 (55.7 mg, 96.28 mol, 19% yield, 95.52% purity, FA) as a yellow solid. LCMS: (M+1: 507.1). ¹H NMR for Ex. 37 is shown in the below NMR table.

### Preparation of 2-[(8aR,19E)-1-ethoxy-11,13-dimethyl-7,8,8a,9,11,17-hexahydro-6H-14,16-(azenometheno)tripyrazolo|3,4-f:3′,4′-j:4″,3″-n]pyrrolo[2,1-c][1,4]oxazacyclopentadecin-3(4H)-yl]ethan-1-ol (Ex. 39)

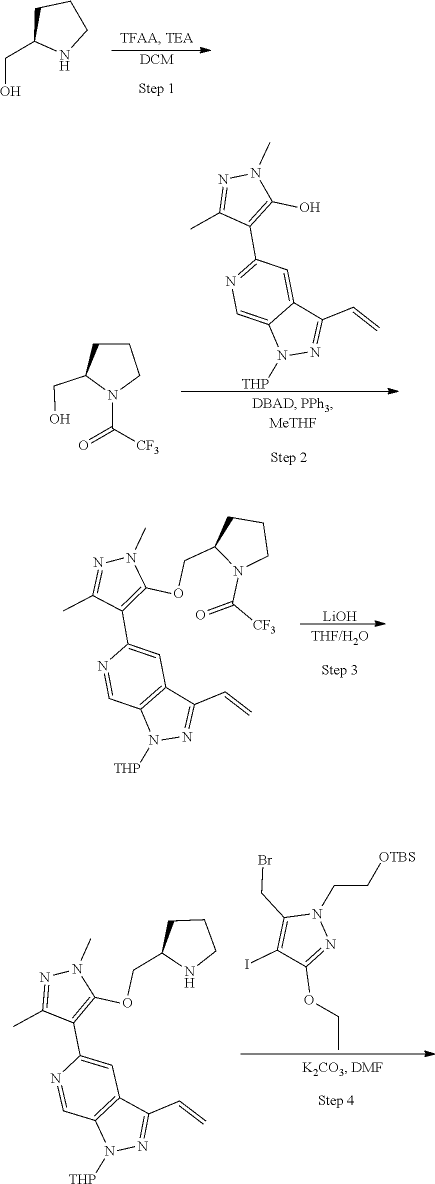

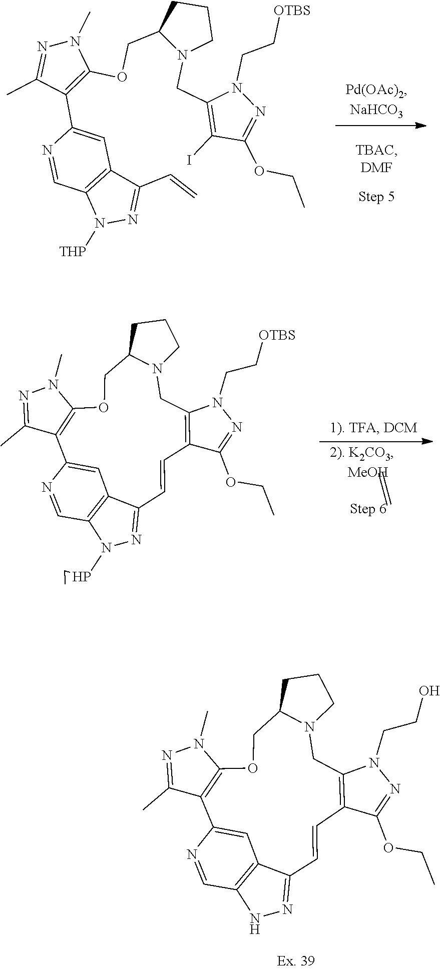

Step 1. To a solution of commercially available [(2R)-pyrrolidin-2-yl]methanol (10.0 g, 98.9 mmol, 1 eq) in DCM (100 mL) was added commercially available (2,2,2-trifluoroacetyl) 2,2,2-trifluoroacetate (31.2 g, 148 mmol, 20.6 mL, 1.5 eq) and TEA (50.0 g, 494 mmol, 5 eq) at 0° C. The mixture was stirred at 0° C. for 18 h. On completion, the reaction mixture was partitioned between dichloromethane (120 mL×3) and water (400 mL). The combined organic phase was dried over anhydrous sodium sulfate, filtered and concentrated to give a residue. The residue was purified by column chromatography (SiO₂, Dichloromethane/Methanol=2/1 to 2/1) to give 2,2,2-trifluoro-1-[(2R)-2-(hydroxymethyl)pyrrolidin-1-yl]ethanone (13.1 g, 66.5 mmol, 67% yield) as a yellow oil. LCMS: (M+1=198.0).

Step 2. A mixture of 2,2,2-trifluoro-1-[(2R)-2-(hydroxymethyl)pyrrolidin-1-yl]ethanone (715 mg, 3.63 mmol, 1 eq), 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (1.60 g, 4.71 mmol, 1.3 eq), PPh₃ (2.09 g, 7.98 mmol, 2.2 eq), DBAD (1.84 g, 7.98 mmol, 2.2 eq) was degassed and purged with N₂ for 3 times, and then the mixture was added 2-MeTHF (20 mL) and stirred at 25° C. for 1.5 h under N₂ atmosphere. On completion, the reaction mixture was partitioned between dichloromethane (30 mL×3) and water (100 mL). The combined organic phase was dried over anhydrous sodium sulfate, filtered and concentrated to give a residue. The residue was purified by column chromatography (SiO₂, Dichloromethane/Methanol=4/1 to 1/10) to give 1-[(2R)-2-[[2,5-dimethyl-4-(1-tetrahydropyran-2-yl-3-vinyl-pyrazolo [3,4-c]pyridin-5-yl)pyrazol-3-yl]oxymethyl]pyrrolidin-1-yl]-2,2,2-trifluoro-ethanone (1.00 g, 2.37 mmol, 88% yield) as a yellow solid. LCMS: (M+1=519.2).

Step 3. To a solution of 1-[(2R)-2-[[2,5-dimethyl-4-(1-tetrahydropyran-2-yl-3-vinyl-pyrazolo[3,4-c]pyridin-5-yl)pyrazol-3-yl]oxymethyl]pyrrolidin-1-yl]-2,2,2-trifluoro-ethanone (1.40 g, 2.70 mmol, 1 eq) in THE (10 mL) and H₂O (2 mL) was added lithium hydroxide hydrate (567 mg, 13.5 mmol, 5 eq). The mixture was stirred at 25° C. for 1 h. On completion, the reaction mixture was partitioned between dichloromethane (30 mL×3) and water (100 mL). The combined organic phase was dried over anhydrous sodium sulfate, filtered and concentrated to give a residue. The residue was purified by column chromatography (SiO₂, Dichloromethane/Methanol=4/1 to 1/10) to give 5-[1,3-dimethyl-5-[[(2R)-pyrrolidin-2-yl]methoxy]pyrazol-4-yl]-1-tetrahydropyran-2-yl-3-vinyl-pyrazolo[3,4-c]pyridine (1.00 g, 2.37 mmol, 88% yield) as a yellow solid. LCMS: (M+1=423.1).

Step 4. A solution of 5-[1,3-dimethyl-5-[[(2R)-pyrrolidin-2-yl]methoxy]pyrazol-4-yl]-1-tetrahydropyran-2-yl-3-vinyl-pyrazolo[3,4-c]pyridine (500 mg, 1.18 mmol, 1 eq) and 2-[5-(bromomethyl)-3-ethoxy-4-iodo-pyrazol-1-yl]ethoxy-tert-butyl-dimethyl-silane, prepared according to the method described in Ex. 1, (579 mg, 1.18 mmol, 1 eq) in DMF (5 mL) was added K₂CO₃ (327 mg, 2.37 mmol, 2 eq). The mixture was stirred at 80° C. for 50 mins. On completion, the reaction mixture was partitioned between ethyl acetate (15 mL×3) and water (50 mL). The combined organic phase was dried over anhydrous sodium sulfate, filtered, and concentrated to give a residue. The residue was purified by column chromatography (SiO₂, Petroleum ether/Tetrahydrofuran=1/1) to give tert-butyl-[2-[5-[[(2R)-2-[[2,5-dimethyl-4-(1-tetrahydropyran-2-yl-3-vinyl-pyrazolo[3,4-c|pyridin-5-yl)pyrazol-3-yl]oxymethyl]pyrrolidin-1-yl]methyl]-3-ethoxy-4-iodo-pyrazol-1-yl]ethoxy]-dimethyl-silane (950 mg, 949 μmol, 80% yield, 83% purity) as a white solid. LCMS: (M+1=831.1).

Step 5. A mixture of tert-butyl-[2-[5-[[(2R)-2-[[2,5-dimethyl-4-(1-tetrahydropyran-2-yl-3-vinyl-pyrazolo[3,4-c]pyridin-5-yl)pyrazol-3-yl]oxymethyl]pyrrolidin-1-yl]methyl]-3-ethoxy-4-iodo-pyrazol-1-yl]ethoxy]-dimethyl-silane (900 mg, 1.08 mmol, 1 eq), TBAC (301 mg, 1.08 mmol, 1 eq), NaHCO₃ (227 mg, 2.71 mmol, 2.5 eq) and Pd(OAc)₂ (48.6 mg, 217 μmol, 0.2 eq) in DMF (10 mL) was degassed and purged with N₂ for 3 times, and then the mixture was stirred at 130° C. for 1.5 h under N₂ atmosphere. On completion, the reaction mixture was partitioned between ethyl acetate (40 mL×3) and water (120 mL). The combined organic phase was dried over anhydrous sodium sulfate, filtered and concentrated to give a residue. The residue was purified by column chromatography (SiO₂, Petroleum ether/Tetrahydrofuran=1/1) to give (8aR,19E)-3-(2-{[tert-butyl(dimethyl)silyl]oxy}ethyl)-1-ethoxy-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecine (410 mg, 583 gmol, 54% yield) as a brown solid. LCMS: (M+1=703.2).

Step 6. To a solution of (8aR,19E)-3-(2-{[tert-butyl(dimethyl)silyl]oxy}ethyl)-1-ethoxy-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecine (400 mg, 569 μmol, 1 eq) in DCM (0.5 mL) was added TFA (0.1 mL). The mixture was stirred at 25° C. for 5 h. On completion, the mixture was concentrated in vacuum to give a brown solid (300 mg, crude).

This brown solid (115 mg, 191 μmol, 1 eq) was dissolved in MeOH (1 mL) and K₂CO₃ (106 mg, 766 mol, 4 eq) was added. The mixture was stirred at 25° C. for 30 mines. On completion, the mixture was filtered. The filtrate was purified by prep-HPLC purification (column: Phenomenex luna C18 150*25 mm*10 um; mobile phase: [water(FA)-ACN]; gradient:4%-34% B over 10 min) to give Ex. 39 (54.18 mg, 98.40 μmol, 51.39% yield, FA) as a yellow solid. LCMS: (M+1=505.2). ¹H NMR for Ex. 39 is shown in the below NMR table.

### Preparation of {[(8aR,19E)-3-(2-hydroxyethyl)-11,13-dimethyl-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile (Ex. 43)

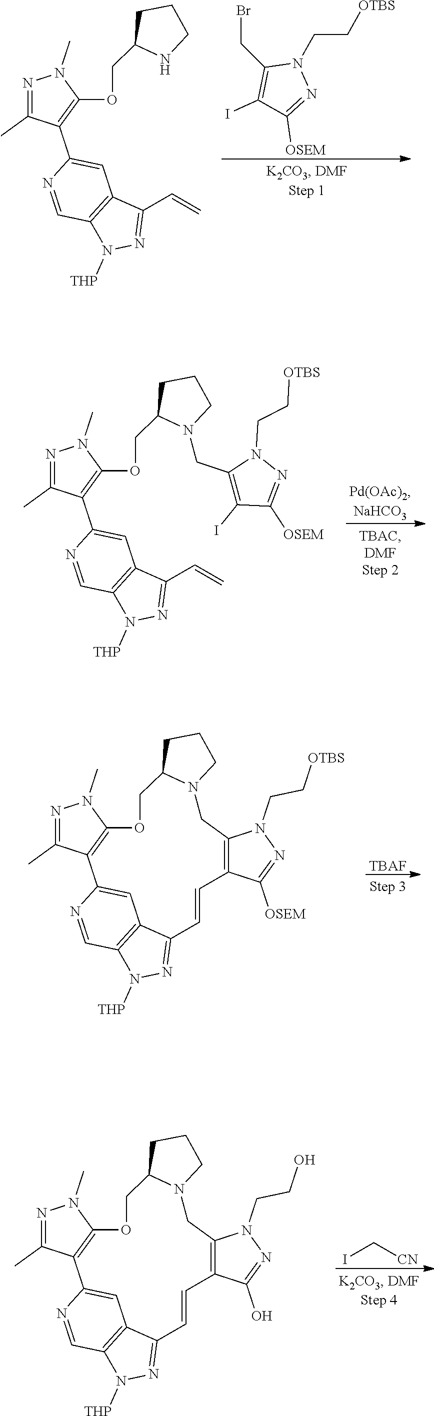

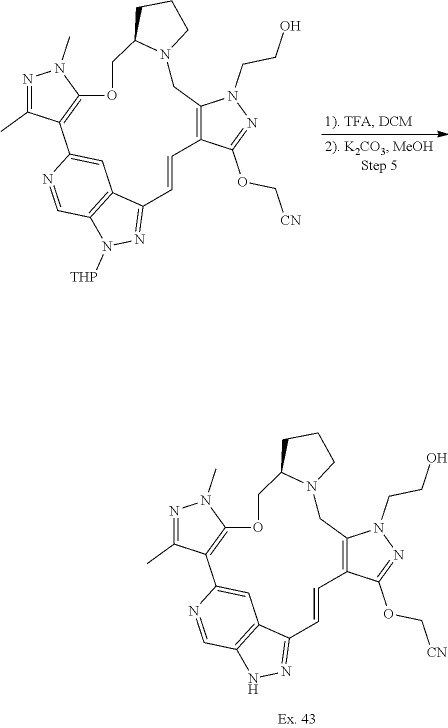

Step 1. A solution of 5-[1,3-dimethyl-5-[[(2R)-pyrrolidin-2-yl]methoxy]pyrazol-4-yl]-1-tetrahydropyran-2-yl-3-vinyl-pyrazolo[3,4-c]pyridine, which was prepared according to the method described in Ex. 39, (700 mg, 1.66 mmol, 1 eq) and 2-[[5-(bromomethyl)-1-[2-[tert-butyl(dimethyl)silyl]oxyethyl]-4-iodo-pyrazol-3-yl]oxymethoxy]ethyl-trimethyl-silane, which was prepared according to the method described in Ex. 42, (980 mg, 1.66 mmol, 1 eq) in DMF (10 mL) was added K₂CO₃ (458 mg, 3.31 mmol, 2 eq). The mixture was stirred at 80° C. for 1.5 h. On completion, the reaction mixture was partitioned between ethyl acetate (80 mL×3) and water (200 mL). The combined organic phase was washed with water (100 mL), dried over anhydrous sodium sulfate, filtered, and concentrated to give a residue. The residue was purified by column chromatography (SiO₂, Petroleum ether/Tetrahydrofuran=1:1) to give tert-butyl-[2-[5-[[(2R)-2-[[2,5-dimethyl-4-(1-tetrahydropyran-2-yl-3-vinyl-pyrazolo[3,4-c]pyridin-5-yl)pyrazol-3-yl]oxymethyl]pyrrolidin-1-yl]methyl]-4-iodo-3-(2-trimethylsilylethoxymethoxy)pyrazol-1-yl]ethoxy]-dimethyl-silane (1.46 g, 1.56 mmol, 94% yield) as a colorless oil. LCMS: (M+1=933.2).

Step 2. A mixture of tert-butyl-[2-[5-[[(2R)-2-[[2,5-dimethyl-4-(1-tetrahydropyran-2-yl-3-vinyl-pyrazolo[3,4-c]pyridin-5-yl)pyrazol-3-yl]oxymethyl]pyrrolidin-1-yl]methyl]-4-iodo-3-(2-trimethylsilylethoxymethoxy)pyrazol-1-yl]ethoxy]-dimethyl-silane (1.46 g, 1.56 mmol, 1 eq), TBAC (435 mg, 1.56 mmol, 1 eq), NaHCO₃ (329 mg, 3.91 mmol, 2.5 eq) and Pd(OAc)₂ (70.3 mg, 314 μmol, 0.2 eq) in DMF (15 mL) was degassed and purged with N₂ for 3 times, and then the mixture was stirred at 130° C. for 1 h under N₂ atmosphere. On completion, the reaction mixture was partitioned between ethyl acetate (80 mL×3) and water (200 mL). The combined organic phase was washed with water (100 mL) and dried over anhydrous sodium sulfate, filtered and concentrated to give a residue. The residue was purified by column chromatography (SiO₂, Petroleum ether/Tetrahydrofuran=1/1) to give (8aR,19E)-3-(2-{[tert-butyl(dimethyl)silyl]oxy}ethyl)-11,13-dimethyl-17-(oxan-2-yl)-1-{[2-(trimethylsilyl)ethoxy]methoxy}-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecine (900 mg, 1.12 mmol, 71% yield) as a brown solid. ¹H NMR (400 MHz, DMSO-d₆) δ=9.19-9.13 (m, 1H), 8.48-8.43 (m, 1H), 7.97-7.91 (m, 1H), 7.18-7.09 (m, 1H), 6.00-5.89 (m, 1H), 5.44 (s, 1H), 5.05-4.97 (m, 1H), 4.27-4.16 (m, 2H), 4.11-4.00 (m, 2H), 3.69 (s, 2H), 3.34-3.28 (m, 5H), 3.22-3.14 (m, 1H), 2.89 (s, 2H), 2.81-2.71 (m, 3H), 2.49-2.43 (m, 3H), 2.32 (br s, 1H), 2.26-2.16 (m, 2H), 1.99 (s, 2H), 1.79-1.73 (m, 2H), 1.64-1.58 (m, 2H), 1.35 (s, 2H), 0.99-0.90 (m, 2H), 0.82-0.77 (m, 9H), −0.02 (s, 6H), −0.05-0.10 (m, 6H), −0.15 (d, J=0.8 Hz, 3H). LCMS: (M+1=805.3).

Step 3. A solution of (8aR,19E)-3-(2-{[tert-butyl(dimethyl)silyl]oxy}ethyl)-11,13-dimethyl-17-(oxan-2-yl)-1-{[2-(trimethylsilyl)ethoxy]methoxy}-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecine (830 mg, 1.03 mmol, 1 eq) in TBAF (4 M, 9 mL, 34.9 eq). The mixture was stirred at 70° C. for 16 h. On completion, the reaction mixture was partitioned between ethyl acetate (30 mL×3) and water (100 mL). The combined organic phase was washed with brine (80 mL) and dried over anhydrous Na₂SO₄, filtered and concentrated to give (8aR,19E)-3-(2-hydroxyethyl)-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol (775 mg, 829 μmol, 80% yield, 60% purity) as a brown solid. LCMS: (M+1=561.2).

Step 4. To a solution of (8aR,19E)-3-(2-hydroxyethyl)-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol (675 mg, 1.20 mmol, 1 eq) in DMF (7 mL) was added commercially available 2-iodoacetonitrile (241 mg, 1.44 mmol, 1.2 eq) and K₂CO₃ (333 mg, 2.41 mmol, 2 eq). The mixture was stirred at 80° C. for 1.5 h. On completion, the reaction mixture was partitioned between ethyl acetate (20 mL×3) and water (70 mL). The combined organic phase was washed with brine (60 mL) and dried over anhydrous sodium sulfate, filtered and concentrated to give a residue. The residue was purified by column chromatography (SiO₂, Petroleum ether/Tetrahydrofuran=3:7 to 2:8) to give {[(8aR,19E)-3-(2-hydroxyethyl)-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile (500 mg, 834 mol, 69% yield) as a brown solid. LCMS: (M+1=600.2).

Step 5. To a solution of {[(8aR,19E)-3-(2-hydroxyethyl)-11,13-dimethyl-17-(oxan-2-yl)-3,4,7,8,8a,9,11,17-octahydro-6H-14,16-(azenometheno)tripyrazolo[3,4-f3′,4′-j:4″,3-n]pyrrolo[2,1-c][1,4]oxazacyclopentadecin-1-yl]oxy}acetonitrile (490 mg, 817 μmol, 1 eq) in DCM (3 mL) was added TFA (13.5 mmol, 1 mL, 16.5 eq). The mixture was stirred at 25° C. for 6 h. On completion, the reaction mixture was concentrated in vacuum to give a yellow solid (400 mg, 637 μmol, 78% yield).

This yellow solid (400 mg, 654 μmol, 1 eq) was dissolved in MeOH (1.5 mL) and K₂CO₃ (271 mg, 1.96 mmol, 3 eq) was added. The mixture was stirred at 25° C. for 10 mins. On completion, the mixture was filtered. The filtrate was purified by prep-HPLC purification (column: Phenomenex luna C18 150*25 mm*10 um; mobile phase: [water(FA)-ACN]; gradient:6%-36% B over 10 min) to give Ex. 43 (12.92 mg, 23.01 mol, 3.52% yield, FA) as a yellow solid. LCMS: (M+1=516.1). ¹H NMR for Ex. 43 is shown in the below NMR table.

### Preparation of {[(8aR,19E)-3-(2-hydroxyethyl)-9,11,13-trimethyl-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile (Ex. 50)

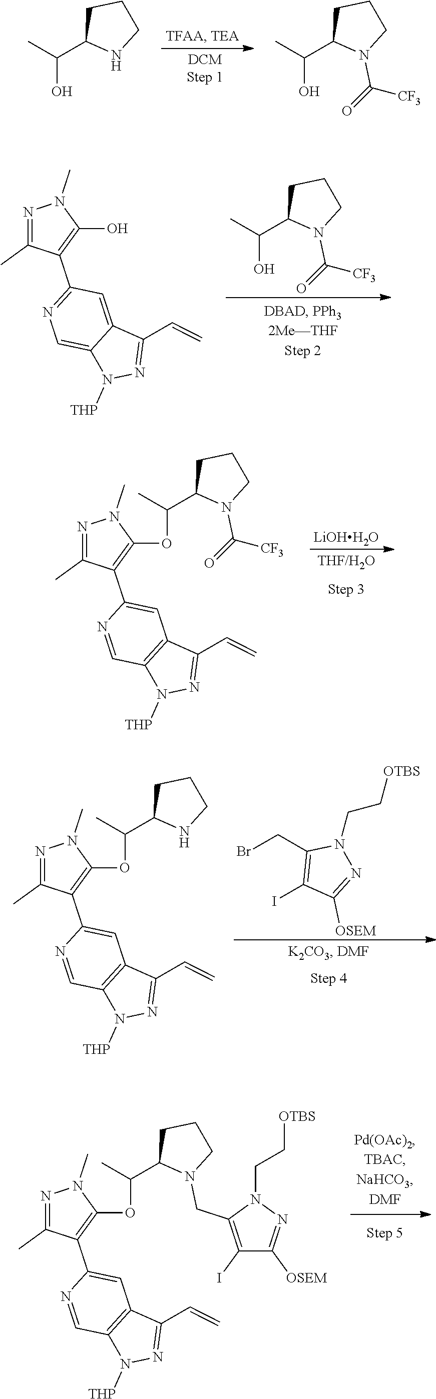

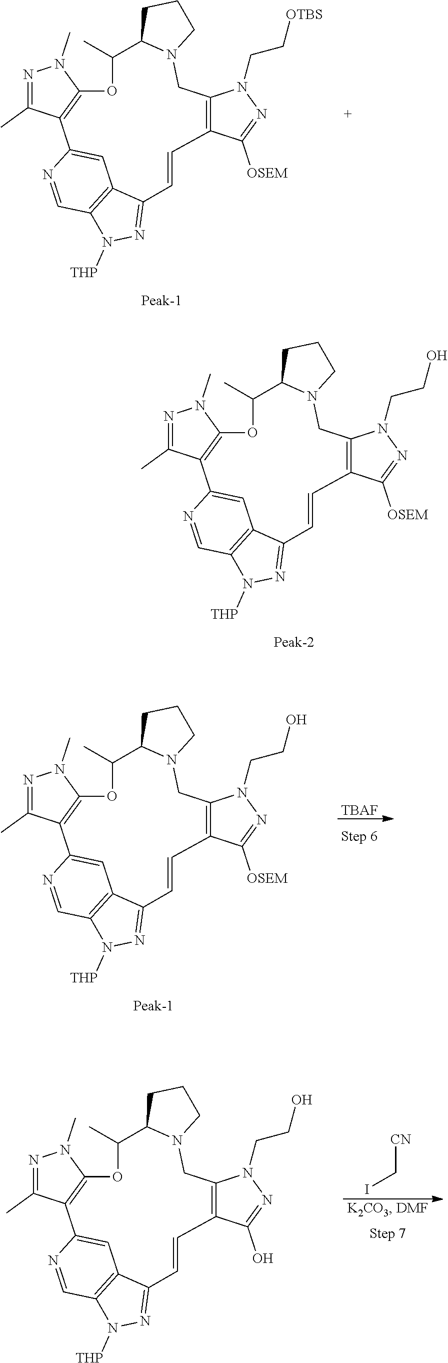

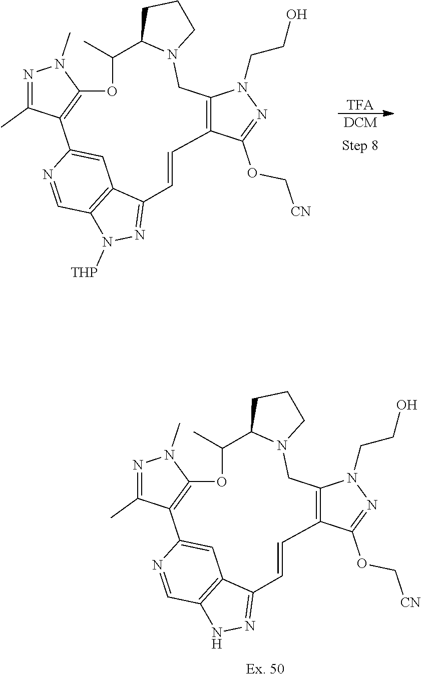

Step 1. To a solution of 1-((R)-pyrrolidin-2-yl)ethan-1-ol, which was prepared according to the method described in Ex. 32 and 33, (1.00 g, 8.68 mmol, 1 eq) in DCM (10 mL) was added TEA (4.39 g, 43.4 mmol, 5 eq) and commercially available (2,2,2-trifluoroacetyl) 2,2,2-trifluoroacetate (2.74 g, 13.0 mmol, 1.5 eq) at 0° C. The mixture was stirred at 0° C. for 16 h. On completion, the reaction mixture was diluted with H₂O (10 mL) and extracted with DCM (20 mL×3). The combined organic layers were dried over Na₂SO₄, filtered and concentrated under reduced pressure to give a residue. The residue was purified by column chromatography (SiO₂, DCM/MeOH=1/0 to 0/1) to give 2,2,2-trifluoro-1-[(2R)-2-(1-hydroxyethyl)pyrrolidin-1-yl]ethanone (1.00 g, 4.74 mmol, 55% yield) as yellow oil. LCMS: (M+1=212.0).

Step 2. The mixture of 2,5-dimethyl-4-(1-tetrahydropyran-2-yl-3-vinyl-pyrazolo[3,4-c]pyridin-5-yl)pyrazol-3-ol, which was prepared according to the method described in Ex. 1, (731 mg, 2.15 mmol, 0.65 eq), 2,2,2-trifluoro-1-[(2R)-2-(1-hydroxyethyl)pyrrolidin-1-yl]ethanone from step 1 (700 mg, 3.31 mmol, 1 eq), PPh₃ (1.91 g, 7.29 mmol, 2.2 eq), and DBAD (1.68 g, 7.29 mmol, 2.2 eq) in 2-methyltetrahydrofuran (7 mL) was degassed and purged with N₂ for 3 times, and then the mixture was stirred at 25° C. for 2 h under N₂ atmosphere. On completion, the reaction mixture was concentrated under reduced pressure to remove solvent. The residue was purified by column chromatography (SiO₂, Petroleum ether/Ethyl acetate=1/0 to 0/1) to give 1-[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]-2,2,2-trifluoro-ethanone (300 mg, 563 mol, 17% yield) as red an oil. LCMS: (M+1=533.2).

Step 3. To a solution of 1-[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]-2,2,2-trifluoro-ethanone (240 mg, 451 mol, 1 eq) in THE (2 mL) and H₂O (0.4 mL) was added LiOH·H₂O (56.7 mg, 1.35 mmol, 3 eq). The mixture was stirred at 25° C. for 2 h. On completion, the reaction mixture was concentrated under reduced pressure to remove solvent. The residue was purified by column chromatography (SiO₂, Petroleum ether/THF=1/0 to 0/1) to give 5-[1,3-dimethyl-5-[1-[(2R)-pyrrolidin-2-yl]ethoxy]pyrazol-4-yl]-1-tetrahydropyran-2-yl-3-vinyl-pyrazolo[3,4-c]pyridine (410 mg, 423 mol, 94% yield, 45% purity) as a yellow oil. LCMS: (M+1=437.2).

Step 4. To a solution of 5-[1,3-dimethyl-5-[1-[(2R)-pyrrolidin-2-yl]ethoxy]pyrazol-4-yl]-1-tetrahydropyran-2-yl-3-vinyl-pyrazolo[3,4-c]pyridine (370 mg, 848 mol, 1 eq) and 2-[[5-(bromomethyl)-1-[2-[tert-butyl(dimethyl)silyl]oxyethyl]-4-iodo-pyrazol-3-yl]oxymethoxy]ethyl-trimethyl-silane, which was prepared according to the method described in Ex. 42, (501 mg, 848 μmol, 1 eq) in DMF (4 mL) was added K₂CO₃ (351 mg, 2.54 mmol, 3 eq). The mixture was stirred at 80° C. for 2 h. On completion, the reaction mixture was diluted with H₂O (12 mL) and extracted with EA (10 mL×3), dried over Na₂SO₄, filtered and concentrated under reduced pressure to give a residue. The residue was purified by column chromatography (SiO₂, Petroleum ether/Ethyl acetate=1/0 to 0/1) to give tert-butyl-[2-[5-[[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]methyl]-4-iodo-3-(2-trimethylsilylethoxymethoxy) pyrazol-1-yl]ethoxy]-dimethyl-silane (380 mg, 401 μmol, 47% yield) as a yellow oil. LCMS: (M+1=947.5).

Step 5. A mixture of tert-butyl-[2-[5-[[(2R)-2-[1-[2,5-dimethyl-4-(1-tetrahydropyran-2-yl-3-vinyl-pyrazolo[3,4-c]pyridin-5-yl)pyrazol-3-yl]oxyethyl]pyrrolidin-1-yl]methyl]-4-iodo-3-(2-trimethylsilylethoxymethoxy)pyrazol-1-yl]ethoxy]-dimethyl-silane (350 mg, 370 μmol, 1 eq), Pd(OAc)₂ (16.6 mg, 73.9 mol, 0.2 eq), NaHCO₃ (77.6 mg, 924 μmol, 2.5 eq) and TBAC (103 mg, 370 mol, 1 eq) in DMF (4 mL) was degassed and purged with N₂ for 3 times, and then the mixture was stirred at 140° C. for 1 h under N₂ atmosphere. On completion, the residue was diluted with H₂O (12 mL) and extracted with EA (10 mL×3). The combined organic layers were dried over Na₂SO₄, filtered and concentrated under reduced pressure to give a residue. The residue was purified by column chromatography (SiO₂, Petroleum ether/THF=1/0 to 0/1) to give Peak-1 (99.0 mg, 121 μmol, 33% yield) as yellow oil and Peak-2 (75.0 mg, 106 mol, 29% yield) as yellow oil. LCMS: (M+1=819.5).

Step 6. A solution of Peak-1 (65.0 mg, 92.2 μmol, 1 eq) in TBAF (1 M, 0.65 mL, 7.05 eq) was stirred at 70° C. for 2 h. On completion, the reaction mixture was concentrated under reduced pressure to remove solvent. The residue was purified by column chromatography (SiO₂, DCM/EA=1/0 to 0/1) to give (8aR,19E)-3-(2-hydroxyethyl)-9,11,13-trimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol (25.0 mg, 43.5 μmol, 47% yield) as a yellow oil. LCMS: (M+1=575.3).

Step 7. To a solution of (8aR,19E)-3-(2-hydroxyethyl)-9,11,13-trimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol (22.0 mg, 38.3 μmol, 1 eq) in DMF (0.9 mL) was added K₂CO₃ (15.9 mg, 115 μmol, 3 eq) and commercially available 2-iodoacetonitrile (7.67 mg, 45.9 mol, 1.2 eq). The mixture was stirred at 80° C. for 2 h. On completion, the reaction mixture was diluted with H₂O (4 mL) and extracted with EA (4 mL×3). The combined organic layers were dried over Na₂SO₄, filtered and concentrated under reduced pressure to give {[(8aR,19E)-3-(2-hydroxyethyl)-9,11,13-trimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile (36.0 mg, 35.2 mol, 92% yield, 60% purity) as a red oil. LCMS: (M+1=614.3).

Step 8. To a solution of {[(8aR,19E)-3-(2-hydroxyethyl)-9,11,13-trimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile (28.0 mg, 45.6 mol, 1 eq) in DCM (0.5 mL) was added TFA (154 mg, 1.35 mmol, 29.5 eq) at 0° C. The mixture was stirred at 25° C. for 2 h. On completion, the reaction mixture was concentrated under reduced pressure to remove solvent. The residue was purified by prep-HPLC (column: Welch Xtimate C18 150*25 mm*5 um; mobile phase: [water(FA)-ACN]; gradient:5%-35% B over 10 min) to give Ex. 50 (0.460 mg, 8.69e−1 μmol, 1.90% yield) as yellow solid. LCMS: (M+1=530.2). ¹H NMR for Ex. 50 is shown in the below NMR table.

### Preparation of 2-[(10R,17E)-12,16-diethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 55)

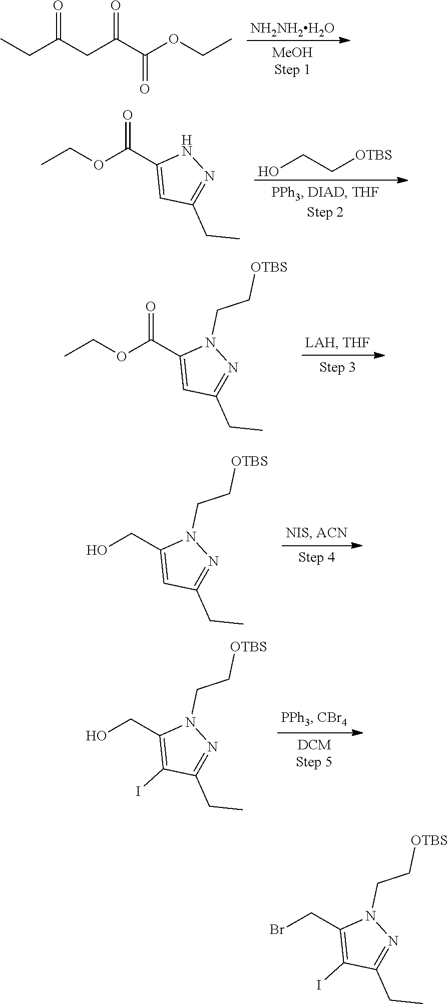

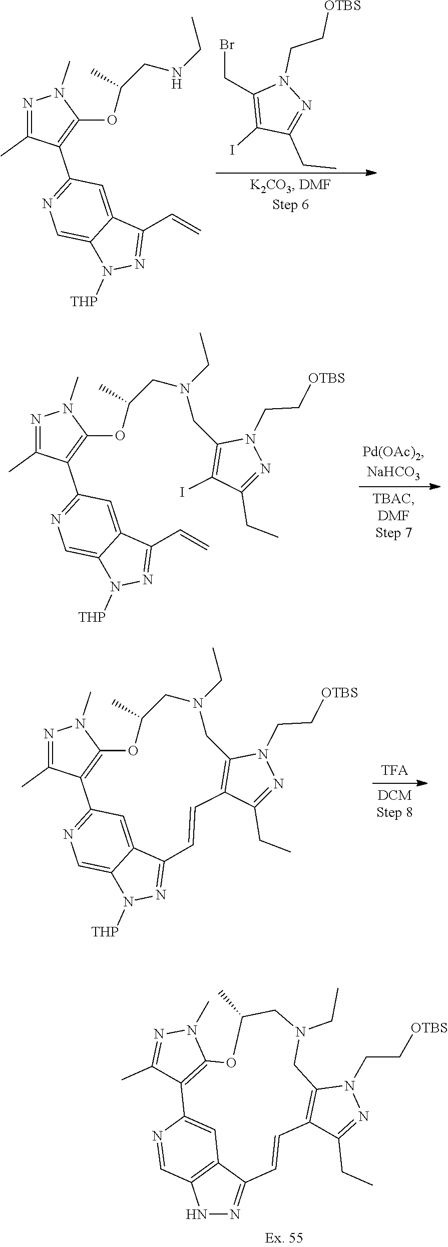

Step 1. To a solution of commercially available ethyl 2,4-dioxohexanoate (30.0 g, 174 mmol, 1 eq) in MeOH (300 mL) was added NH₂NH₂·H₂O (9.59 g, 191 mmol, 9.30 mL, 1.1 eq) at 0° C. The mixture was stirred at 25° C. for 3 hr. On completion, the mixture was concentrated to give ethyl 3-ethyl-1H-pyrazole-5-carboxylate (32.0 g, crude) as colorless solid. LCMS: (M+1: 169.0).

Step 2. A mixture of ethyl 3-ethyl-1H-pyrazole-5-carboxylate (27.0 g, 160 mmol, 1 eq), commercially available 2-[tert-butyl(dimethyl)silyl]oxyethanol (42.4 g, 240 mmol, 1.5 eq), PPh₃ (92.6 g, 353 mmol, 2.2 eq) in THF (270 mL) was stirred at 25° C. for 30 min. Then to the mixture was added DIAD (71.4 g, 353 mmol, 2.2 eq) at 0° C. and then the mixture was stirred at 25° C. for 3 h under N₂ atmosphere. On completion, the mixture was concentrated to give a residue. The residue was purified by column chromatography (SiO₂, Petroleum ether/Ethyl acetate=1:0 to 90:10) to give ethyl 2-[2-[tert-butyl(dimethyl)silyl]oxyethyl]-5-ethyl-pyrazole-3-carboxylate (26.0 g, 79.6 mmol, 50% yield) as colorless oil. ¹H NMR (400 MHz, CDCl₃) S=6.60 (s, 1H), 4.63 (t, J=5.6 Hz, 2H), 4.32-4.25 (m, 2H), 3.90 (t, J=5.6 Hz, 2H), 2.61 (q, J=7.6 Hz, 2H), 1.37-1.30 (m, 3H), 1.25-1.18 (m, 3H), 0.80-0.75 (m, 9H), −0.09-0.15 (m, 6H). LCMS: (M+1: 327.2).

Step 3. To a solution of ethyl 2-[2-[tert-butyl(dimethyl)silyl]oxyethyl]-5-ethyl-pyrazole-3-carboxylate (24.0 g, 73.5 mmol, 1 eq) in THE (240 mL) was added LiAlH₄ (2.5 M, 29.4 mL, 1 eq) at 0° C. The mixture was stirred at 0° C. for 2 h. On completion, the mixture was quenched with MeOH (50 mL) at 0° C., and concentrated to give a residue. The residue was purified by column chromatography (SiO₂, Petroleum ether/Ethyl acetate=1:0 to 0:1) to give [2-[2-[tert-butyl(dimethyl)silyl]oxyethyl]-5-ethyl-pyrazol-3-yl]methanol (13.8 g, 48.5 mmol, 66% yield) as colorless oil. LCMS: (M+1: 285.0).

Step 4. To a solution of [2-[2-[tert-butyl(dimethyl)silyl]oxyethyl]-5-ethyl-pyrazol-3-yl]methanol (12.8 g, 45.0 mmol, 1 eq) in ACN (130 mL) was added NIS (9.11 g, 40.5 mmol, 0.9 eq) at 0° C. The mixture was stirred at 0° C. for 2 h. On completion, the mixture was quenched with sat. Na₂SO₃ (200 mL) at 0° C. and extracted with ethyl acetate (100 mL×3). The combined organic phase was dried over anhydrous Na₂SO₄, filtered and the filtrate was concentrated to give a residue. The residue was purified by column chromatography (SiO₂, Petroleum ether/Ethyl acetate=1:0 to 1:1) to give [2-[2-[tert-butyl(dimethyl)silyl]oxyethyl]-5-ethyl-4-iodo-pyrazol-3-yl]methanol (16.8 g, 40.9 mmol, 91% yield) as yellow oil. LCMS: (M+1: 411.0).

Step 5. To a solution of [2-[2-[tert-butyl(dimethyl)silyl]oxyethyl]-5-ethyl-4-iodo-pyrazol-3-yl]methanol (15.8 g, 38.5 mmol, 1 eq), PPh₃ (12.1 g, 46.2 mmol, 1.2 eq) in DCM (158 mL) was added CBr₄ (15.3 g, 46.2 mmol, 1.2 eq) at 0° C. The mixture was stirred at 0° C. for 2 h. On completion, the mixture was diluted with water (150 mL) and extracted with DCM (70 mL×3). The combined organic phase dried over Na₂SO₄, filtered and the filtrate was concentrated to give a residue. The residue was purified by column chromatography (SiO₂, Petroleum ether/Ethyl acetate=1:0 to 80:20) to give 2-[5-(bromomethyl)-3-ethyl-4-iodo-pyrazol-1-yl]ethoxy-tert-butyl-dimethyl-silane (11.8 g, 24.9 mmol, 65% yield) as yellow oil. ¹H NMR (400 MHz, CDCl₃) δ=4.60-4.51 (m, 2H), 4.30 (t, J=4.8 Hz, 2H), 4.00-3.83 (m, 2H), 2.59 (q, J=7.6 Hz, 2H), 1.22 (t, J=7.6 Hz, 3H), 0.79 (s, 10H), −0.11 (s, 6H). LCMS: (M+1: 474.6).

Step 6. To a solution of (2R)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine, which was prepared according to the method described in Ex. 36, (300 mg, 706 μmol, 1 eq), 2-[5-(bromomethyl)-3-ethyl-4-iodo-pyrazol-1-yl]ethoxy-tert-butyl-dimethyl-silane (334 mg, 706 mol, 1 eq) in DMF (5 mL) was added K₂CO₃ (292 mg, 2.12 mmol, 3 eq). The mixture was stirred at 80° C. for 1 h. On completion, the mixture was diluted with water (50 mL) and extracted with ethyl acetate (20 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:0 to 80:20) to give (2R)-N-[[2-[2-[tert-butyl(dimethyl)silyl]oxyethyl]-5-ethyl-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (250 mg, 306 mol, 43% yield) as yellow oil. LCMS: (M+1: 817.5).

Step 7. A mixture of (2R)-N-[[2-[2-[tert-butyl(dimethyl)silyl]oxyethyl]-5-ethyl-4-iodo-pyrazol-3-yl]methyl]-2-[2,5-dimethyl-4-(1-tetrahydropyran-2-yl-3-vinyl-pyrazolo [3,4-c]pyridin-5-yl)pyrazol-3-yl]oxy-N-ethyl-propan-1-amine (230 mg, 281 mol, 1 eq), TBAC (78.2 mg, 281 mol, 1 eq), NaHCO₃ (59.1 mg, 703 mol, 2.5 eq) and Pd(OAc)₂ (12.6 mg, 56.3 μmol, 0.2 eq) in DMF (2.5 mL) was degassed and purged with N₂ for 3 times, and then the mixture was stirred at 100° C. for 2 h under N₂ atmosphere. On completion, the mixture was diluted with water (50 mL) and extracted with ethyl acetate (20 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:0 to 0:1) to give (10R,17E)-14-(2-{[tert-butyl(dimethyl)silyl]oxy}ethyl)-12,16-diethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (83.0 mg, 120 μmol, 43% yield) as yellow oil. LCMS: (M+1: 689.2).

Step 8. To a solution of (10R,17E)-14-(2-{[tert-butyl(dimethyl)silyl]oxy}ethyl)-12,16-diethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (70.0 mg, 101 μmol, 1 eq) in DCM (2.5 mL) was added TFA (1.32 g, 11.6 mmol, 114 eq). The mixture was stirred at 25° C. for 1 h. On completion, the mixture was concentrated to give a residue. The residue was purified by prep-HPLC purification (Phenomenex luna C18 150*25 mm*10 um; mobile phase: [water(FA)-ACN]; gradient:5%-35% B over 15 min) to give Ex. 55 (10.55 mg, 21.50 μmol, 21.17% yield) as white solid. LCMS: (M+1: 491.1). ¹H NMR for Ex. 55 is shown in the below NMR table.

### Preparation of {[(10R,17E)-12-ethyl-14-[(2S)-1-hydroxypropan-2-yl]-6,8,10-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 58)

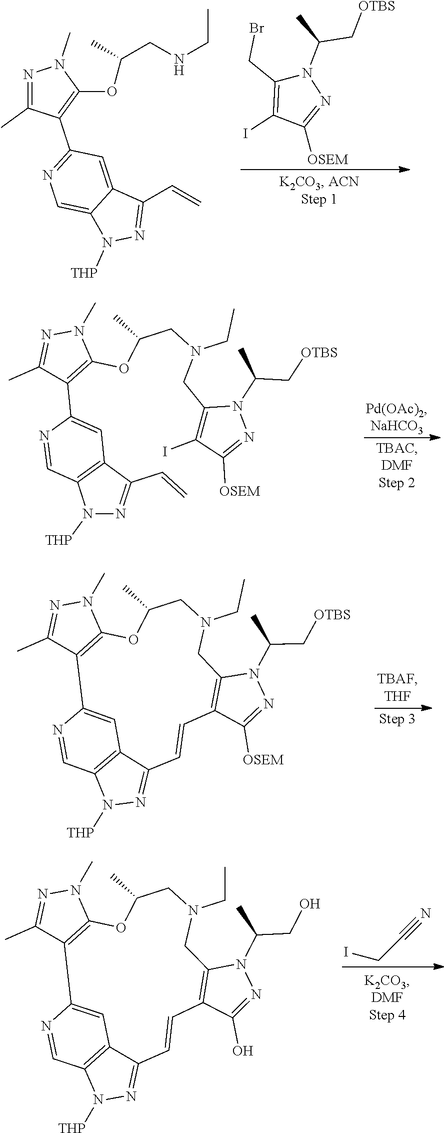

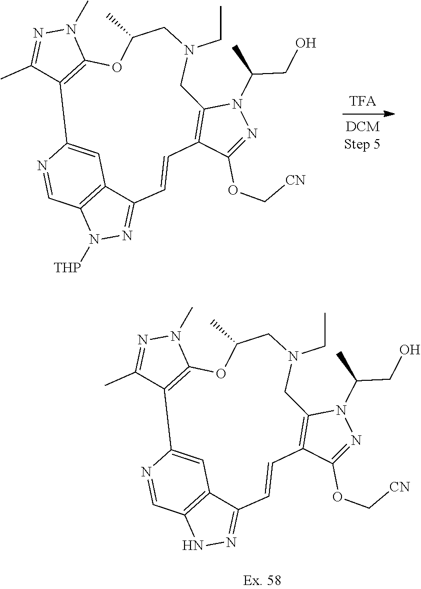

Step 1. A solution of (2S)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine, which was prepared according to the method described in Ex. 36, (300 mg, 707 mol, 1 eq), 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, (471 mg, 777 mol, 1.1 eq) and K₂CO₃ (293 mg, 2.12 mmol, 3 eq) in ACN (7 mL), was stirred at 60° C. for 16 h. On completion, the reaction mixture was concentrated in vacuo. The residue was purified by column chromatography on silica gel (4 g silica gel, EA in Petroleum ether from 0% to 100%) to give (2S)-N-[[2-[(S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (400 mg, 399 μmol, 56% yield, 94% purity) as a yellow solid. LCMS: (M+1: 949.2).

Step 2. To a solution of (2S)-N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-12,5-dimethyl-4-(1-tetrahydropyran-2-yl-3-vinyl-pyrazolo[3,4-c]pyridin-5-yl)pyrazol-3-yl]oxy-N-ethyl-propan-1-amine (380 mg, 400 mol, 1 eq), NaHCO₃ (84.1 mg, 1.00 mmol, 2.5 eq), TBAC (111 mg, 400 mol, 1 eq), diacetoxypalladium (18.0 mg, 80.1 mol, 0.2 eq) in DMF (38 mL) was degassed and purged with N₂ for 3 times, and then the mixture was stirred at 120° C. for 2 h under N₂ atmosphere. On completion, the mixture was poured into H₂O (120 mL) and extracted with EA (30 mL×3). The combined organic phase was washed with H₂O (30 mL×3) and dried over anhydrous sodium sulfate, filtered and concentrated to give a residue. The residue was purified by column chromatography (SiO₂, Petroleum ether/THF=3:1 to 0:1) to give (10R,17E)-14-[(2S)-1-{[tert-butyl(dimethyl)silyl]oxy}propan-2-yl]-12-ethyl-6,8,10-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (327 mg, 354 mol, 88% yield, 89% purity) as a yellow solid. LCMS: (M+1: 821.4).

Step 3. To a solution of (10R,17E)-14-[(2S)-1-{[tert-butyl(dimethyl)silyl]oxy}propan-2-yl]-12-ethyl-6,8,10-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (300 mg, 365 μmol, 1 eq) in TBAF (1 M in THF, 2.19 mL, 6 eq), the mixture was stirred at 70° C. for 16 h. On completion, the reaction mixture was concentrated in vacuo to give (10R,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (300 mg, 348 μmol, 95% yield, 67% purity) as a black solid. LCMS: (M+1: 577.2).

Step 5. To a mixture of (10R,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (269 mg, 312 mol, 1 eq) and K₂CO₃ (129 mg, 936 mol, 3 eq) in DMF (3 mL) was added commercially available 2-iodoacetonitrile (52.1 mg, 312 μmol, 1 eq) at 0° C. The mixture was stirred at 25° C. for 1 h. On completion, the mixture was diluted with water (10 mL) and extracted with ethyl acetate (2 mL×3). The combined organic phase was washed with saturated sodium chloride solution (2 mL×3) and dried over Na₂SO₄, filtered and the filtrate was concentrated to give {[(10R,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (150 mg, 170 μmol, 54% yield, 70% purity) as a black solid. LCMS: (M+1: 616.3).

Step 6. To a solution of {[(10R,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (130 mg, 219 mol, 1 eq) in DCM (1.5 mL) was added TFA (4.04 mmol, 0.3 mL, 18.4 eq). The mixture was stirred at 25° C. for 1 h. On completion, the reaction mixture was concentrated in vacuo. The residue was purified by Prep-HPLC (column: Phenomenex luna C18 150*25 mm*10 um; mobile phase: [water(FA)-ACN]; gradient:7%-37% B over 15 min) to give Ex. 58 (45.05 mg, 78.49 mol, 36% yield, 96.8% purity, FA) as a white solid. LCMS: (M+1: 532.2). ¹H NMR for Ex. 58 is shown in the below NMR table.

### Preparation of 2-[(10S,17E)-12,16-diethyl-6,8,10-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 62)

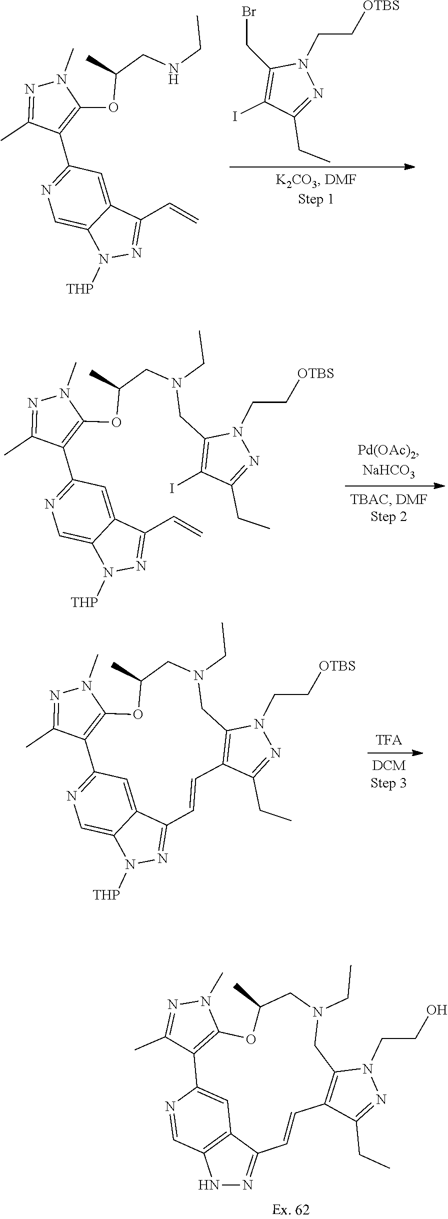

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine, which was prepared according to the method described in Ex. 23, (500 mg, 1.18 mmol, 1 eq), 2-[5-(bromomethyl)-3-ethyl-4-iodo-pyrazol-1-yl]ethoxy-tert-butyl-dimethyl-silane, which was prepared according to the method described in Ex. 55, (557 mg, 1.18 mmol, 1 eq) in DMF (5 mL) was added K₂CO₃ (488 mg, 3.53 mmol, 3 eq). The mixture was stirred at 80° C. for 1 h. On completion, the mixture was diluted with water (100 mL) and extracted with ethyl acetate (20 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:0 to 80:20) to give (2S)-N-[[2-[2-[tert-butyl(dimethyl)silyl]oxyethyl]-5-ethyl-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (265 mg, 324 μmol, 28% yield) as yellow oil. LCMS: (M+1: 817.1).

Step 2. A mixture of (2S)-N-[[2-[2-[tert-butyl(dimethyl)silyl]oxyethyl]-5-ethyl-4-iodo-pyrazol-3-yl]methyl]-2-[2,5-dimethyl-4-(1-tetrahydropyran-2-yl-3-vinyl-pyrazolo [3,4-c]pyridin-5-yl)pyrazol-3-yl]oxy-N-ethyl-propan-1-amine (235 mg, 287 mol, 1 eq), TBAC (79.9 mg, 287 μmol, 1 eq), NaHCO₃ (60.4 mg, 719 μmol, 2.5 eq) and Pd(OAc)₂ (12.9 mg, 57.5 μmol, 0.2 eq) in DMF (2.5 mL) was degassed and purged with N₂ for 3 times, and then the mixture was stirred at 80° C. for 2 h under N₂ atmosphere. On completion, the mixture was diluted with water (50 mL) and extracted with ethyl acetate (20 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:0 to 0:1) to give (10S,17E)-14-(2-{[tert-butyl(dimethyl)silyl]oxy}ethyl)-12,16-diethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (85.0 mg, 123 μmol, 43% yield) as yellow oil. LCMS: (M+1: 689.4).

Step 3. To a solution of (10S,17E)-14-(2-{[tert-butyl(dimethyl)silyl]oxy}ethyl)-12,16-diethyl-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (75.0 mg, 108 mol, 1 eq) in DCM (3 mL) was added TFA (1.41 g, 12.4 mmol, 114 eq). The mixture was stirred at 25° C. for 1 h. On completion, the mixture was concentrated to give a residue. The residue was purified by prep-HPLC purification (Phenomenex luna C18 150*25 mm*10 um; mobile phase: [water(FA)-ACN]; gradient:4%-34% B over 10 min) to give Ex. 62 (14.23 mg, 29.01 μmol, 26.65% yield) as yellow solid. LCMS: (M+1: 491.1). ¹H NMR for Ex. 62 is shown in the below NMR table.

### Preparation of {[(8aR,19E)-3-[(2S)-1-hydroxypropan-2-yl]-11,13-dimethyl-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-yl]oxy}acetonitrile (Ex. 64)

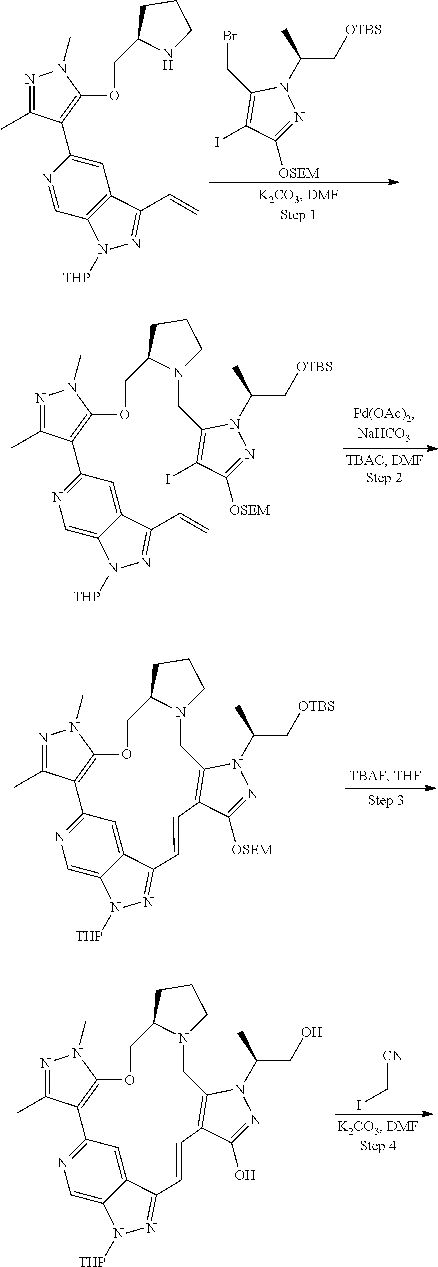

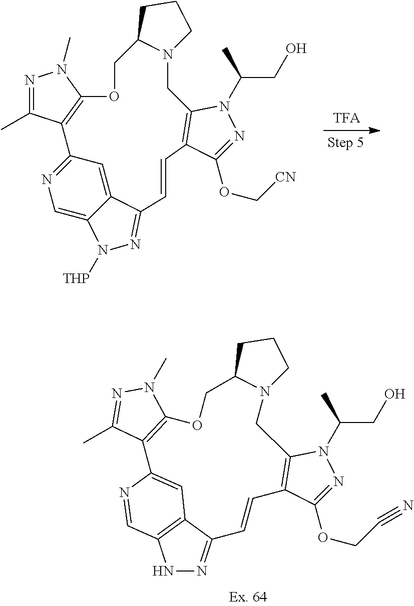

Step 1. To a solution of 5-[1,3-dimethyl-5-[[(2R)-pyrrolidin-2-yl]methoxy]pyrazol-4-yl]-1-tetrahydropyran-2-yl-3-vinyl-pyrazolo[3,4-c]pyridine, which was prepared according to the method described in Ex. 39, (600 mg, 1.42 mmol, 1 eq) and 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, (1.03 g, 1.70 mmol, 1.2 eq) in DMF (6 mL) was added K₂CO₃ (588 mg, 4.26 mmol, 3 eq). The mixture was stirred at 80° C. for 1 hr. On completion, the reaction mixture was quenched by addition of H₂O (8 mL) at 25° C. and extracted with EA (8 mL×3). The combined organic layers were washed with H₂O (10 mL×2), dried over Na₂SO₄, filtered and concentrated under reduced pressure to give a residue. The residue was purified by CombiFlash (4 g silica gel column, THE in PE from 0-100%) to give tert-butyl-[(2S)-2-[5-[[(2R)-2-[[2,5-dimethyl-4-(1-tetrahydropyran-2-yl-3-vinyl-pyrazolo[3,4-c]pyridin-5-yl)pyrazol-3 yl]oxymethyl]pyrrolidin-1-yl]methyl]-4-iodo-3-(2-trimethylsilylethoxymethoxy)pyrazol-1-yl]propoxy]-dimethyl-silane (1.00 g, 1.06 mmol, 74% yield) as a brown oil. LCMS: (M+1: 947.4).

Step 2. A mixture of tert-butyl-[(2S)-2-[5-[[(2R)-2-[[2,5-dimethyl-4-(1-tetrahydropyran-2-yl-3-vinyl-pyrazolo [3,4-c]pyridin-5-yl)pyrazol-3-yl]oxymethyl]pyrrolidin-1-yl]methyl]-4-iodo-3-(2-trimethylsilylethoxymethoxy)pyrazol-1-yl]propoxy]-dimethyl-silane (950 mg, 1.00 mmol, 1 eq), TBAC (278 mg, 1.00 mmol, 280 μL, 1 eq), NaHCO₃ (210 mg, 2.51 mmol, 2.5 eq) and Pd(OAc)₂ (45.0 mg, 200 μmol, 0.2 eq) in DMF (10 mL) was degassed and purged with N₂ for 3 times, and then the mixture was stirred at 130° C. for 1 hr under N₂ atmosphere. On completion, the reaction mixture was quenched by addition H₂O (30 mL) at 25° C. and extracted with EA (15 mL×3). The combined organic layers were washed with H₂O (20 mL×2), dried over Na₂SO₄, filtered and concentrated under reduced pressure to give a residue. The residue was purified by CombiFlash (4 g silica gel column, THE in PE from 0-100%) to give (8aR,19E)-3-[(2S)-1-{[tert-butyl(dimethyl)silyl]oxy}propan-2-yl]-11,13-dimethyl-17-(oxan-2-yl)-1-{[2-(trimethylsilyl)ethoxy]methoxy}-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecine (670 mg, 817 μmol, 82% yield) as a brown oil. ¹H NMR (400 MHz, CDCl₃-d) 6=9.10 (br s, 1H), 8.50 (s, 1H), 7.93 (d, J=16.8 Hz, 1H), 7.31 (dd, J=2.4, 16.8 Hz, 1H), 5.82-5.73 (m, 1H), 5.49 (s, 1H), 5.04-4.96 (m, 1H), 4.53-4.37 (m, 2H), 4.12-4.01 (m, 3H), 3.89 (t, J=8.3 Hz, 2H), 3.76 (s, 3H), 3.50-3.42 (m, 1H), 3.32-3.01 (m, 4H), 2.97-2.90 (m, 1H), 2.63 (s, 3H), 2.60-2.48 (m, 2H), 2.33-2.22 (m, 1H), 2.22-2.13 (m, 3H), 2.07-1.94 (m, 1H), 1.44 (dd, J=2.4, 4.4 Hz, 3H), 1.41 (d, J=6.8 Hz, 2H), 1.03 (dt, J=3.8, 8.4 Hz, 2H), 0.81 (d, J=8.0 Hz, 15H), 0.73-0.59 (m, 2H), 0.04-0.01 (m, 9H). LCMS: (M+1: 819.3).

Step 3. To a solution of (8aR,19E)-3-[(2S)-1-{[tert-butyl(dimethyl)silyl]oxy}propan-2-yl]-11,13-dimethyl-17-(oxan-2-yl)-1-{[2-(trimethylsilyl)ethoxy]methoxy}-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecine (600 mg, 732 mol, 1 eq) in THE (6 mL) was added TBAF (1 M, 4.39 mL, 6 eq). The mixture was stirred at 70° C. for 3 hr. On completion, the reaction mixture was quenched by addition of H₂O (10 mL) at 25° C. and extracted with EA (8 mL×3). The combined organic layers were washed with H₂O (10 mL×2), dried over Na₂SO₄, filtered and concentrated under reduced pressure to give a residue. The residue was purified by CombiFlash (4 g silica gel column, THE in PE from 0-100%) to give (8aR,19E)-3-[(2S)-1-hydroxypropan-2-yl]-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol (160 mg, 278 mol, 38% yield) as a brown oil. LCMS: (M+1: 575.2).

Step 4. To a solution of (8aR,19E)-3-1(2S)-1-hydroxypropan-2-yl]-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol (65.0 mg, 0.113 mmol, 1 eq), 2-iodoacetonitrile (28.3 mg, 0.169 mmol, 1.5 eq) in DMF (1 mL) was added K₂CO₃ (46.9 mg, 339 μmol, 3 eq). The mixture was stirred at 80° C. for 1 hr. On completion, the mixture was diluted with water (50 mL) and extracted with ethyl acetate (20 mL×3). The combined organic phase was washed with water (20 mL×2) and dried over Na₂SO₄, filtered and the filtrate was concentrated to give a residue. The residue was purified by column chromatography (SiO₂, DCM/MeOH=1:0 to 80:20) to give {[(8aR,19E)-3-[(2S)-1-hydroxypropan-2-yl]-11,13-dimethyl-17-(oxan-2-yl)-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecin-1-yl]oxy}acetonitrile (50.0 mg, 81.4 mol, 72% yield) as yellow oil. LCMS: (M+1: 614.2).

Step 5. To a solution of {[(8aR,19E)-3-[(2S)-1-hydroxypropan-2-yl]-11,13-dimethyl-17-(oxan-2-yl)-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecin-1-yl]oxy}acetonitrile (45.0 mg, 73.3 μmol, 1 eq) in DCM (3 mL) was added TFA (944 mg, 8.29 mmol, 615 μL, 113 eq). The mixture was stirred at 25° C. for 1 hr. On completion, the mixture was filtered and concentrated to give a residue. The residue was purified by prep-HPLC purification (Phenomenex luna C18 150*25 mm*10 um; mobile phase: [water(FA)-ACN]; gradient:6%-36% B over 10 min) to give Ex. 64 (8.73 mg, 16.48 μmol, 22.48% yield) as white solid. LCMS: (M+1: 530.1). ¹H NMR for Ex. 64 is shown in the below NMR table.

### Preparation of (2S)-2-[(8aR,19E)-1-ethoxy-11,13-dimethyl-7,8,8a,9,11,17-hexahydro-6H-14,16-(azenometheno)tripyrazolo[3,4-f:3′,4′-j:4″,3″-n]pyrrolo[2,1-c][1,4]oxazacyclopentadecin-3(4H)-yl]propan-1-ol (Ex. 65)

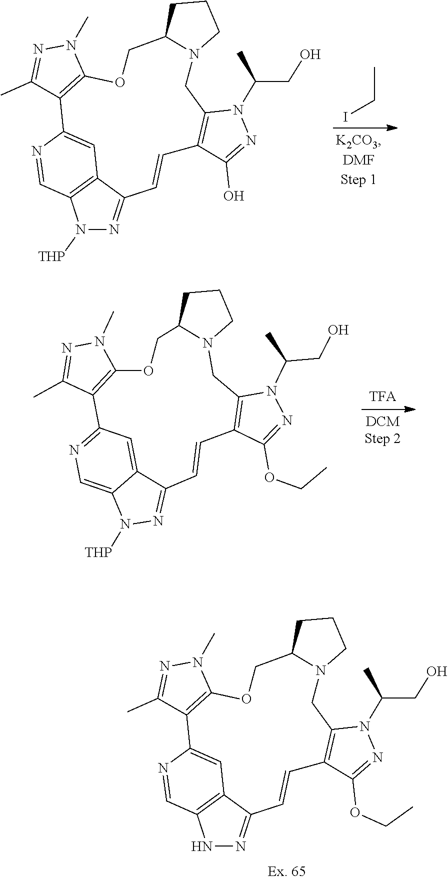

Step 1. To a solution of (8aR,19E)-3-[(2S)-1-hydroxypropan-2-yl]-11,13-dimethyl-17-(oxan-2-yl)-3,4,7,8,8a,9,11,17-octahydro-6H-14,16-(azenometheno)tripyrazolo[3,4-f:3′,4′-j:4″,3″-n]pyrrolo[2,1-c][1,4]oxazacyclopentadecin-1-ol, which was prepared according to the method described in Ex. 64, (65.0 mg, 113 mol, 1 eq), CH₃CH₂I (26.4 mg, 169 mol, 1.5 eq) in DMF (1 mL) was added K₂CO₃ (46.9 mg, 339 μmol, 3 eq). The mixture was stirred at 80° C. for 1 h. On completion, the mixture was diluted with water (50 mL) and extracted with ethyl acetate (10 mL×3). The combined organic phase was washed with water (20 mL×2) and dried over Na₂SO₄, filtered and the filtrate was concentrated to give a residue. The residue was purified by column chromatography (SiO₂, DCM/MeOH=1:0 to 80:20) to give (2S)-2-[(8aR,19E)-1-ethoxy-11,13-dimethyl-17-(oxan-2-yl)-4,7,8,8a,9,17-hexahydro-3H,6H,1H-14,16-(azenometheno)tripyrazolo[3,4-f:3′,4′-j:4″,3″-n]pyrrolo[2,1-c][1,4]oxazacyclopentadecin-3-yl]propan-1-ol (60.0 mg, 99.5 μmol, 88% yield) as yellow oil. LCMS: (M+1: 603.2).

Step 2. To a solution of (2S)-2-[(8aR,19E)-1-ethoxy-11,13-dimethyl-17-(oxan-2-yl)-4,7,8,8a,9,17-hexahydro-3H,6H,11H-14,16-(azenometheno)tripyrazolo[3,4-f:3′,4′-j:4″,3″-n]pyrrolo[2,1-c][1,4]oxazacyclopentadecin-3-yl]propan-1-ol (50.0 mg, 82.9 mol, 1 eq) in DCM (2.5 mL) was added TFA (9.37 mmol, 696 μL, 113 eq). The mixture was stirred at 25° C. for 1 h. On completion, the mixture was filtered and concentrated to give a residue. The residue was purified by prep-HPLC purification (Phenomenex luna C18 150*25 mm*10 um; mobile phase: [water(FA)-ACN]; gradient:7%-37% B over 10 min) to give Ex. 65 (6.03 mg, 11.63 μmol, 14.02% yield) as yellow solid. LCMS: (M+1: 519.1). ¹H NMR for Ex. 65 is shown in the below NMR table.

### Preparation of {[(10S,17E)-12-ethyl-14-[(2S)-1-hydroxypropan-2-yl]-6,8,10-trimethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 67)

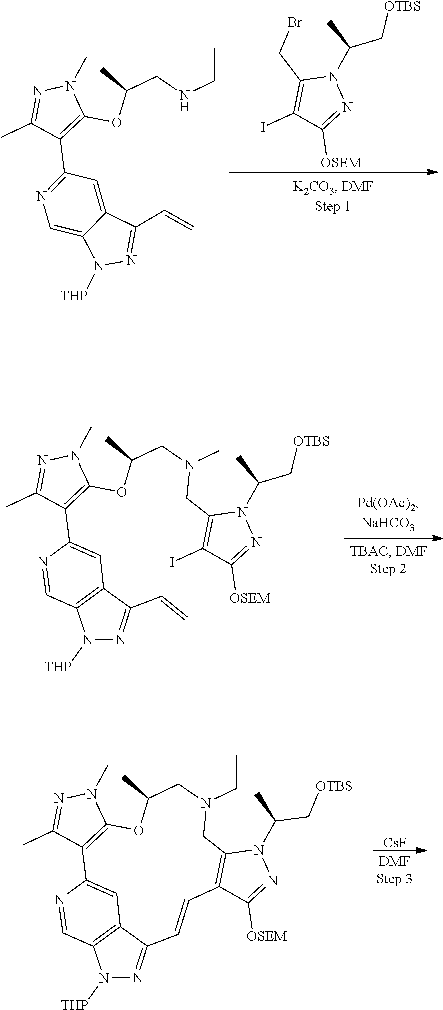

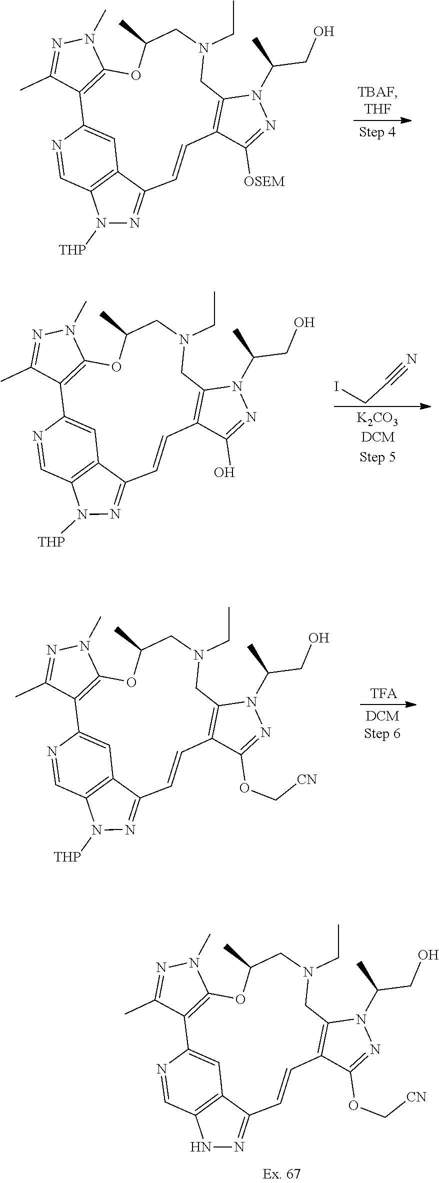

Step 1. To a solution of (2S)-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine, which was prepared according to the method described in Ex. 23, (200 mg, 471 mol, 1 eq) and 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, (199 mg, 329 μmol, 0.7 eq) in DMF (4 mL) was added K₂CO₃ (195 mg, 1.41 mmol, 3 eq). The mixture was stirred at 80° C. for 2 h. On completion, the mixture was diluted with water (20 mL) and extracted with ethyl acetate (25 mL×3). The combined organic phase was dried over anhydrous sodium sulfate, filtered and concentrated to give a residue. The residue was purified by column chromatography (SiO₂, Petroleum ether/Ethyl acetate=2:1 to 1:1) to give (2S)-N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (210 mg, 214 mol, 45% yield, 97% purity) as a yellow liquid. LCMS: (M+1=949.1).

Step 2. A mixture of (2S)-N-[[2-[(1S)-2-[tert-butyl(dimethyl)silyl]oxy-1-methyl-ethyl]-4-iodo-5-(2-trimethylsilylethoxymethoxy)pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-ethyl-propan-1-amine (190 mg, 200 mol, 1 eq), Pd(OAc)₂ (8.99 mg, 40.0 μmol, 0.2 eq), TBAC (55.6 mg, 200 mol, 1 eq) and NaHCO₃ (42.0 mg, 500 μmol, 2.5 eq) in DMF (2 mL) was degassed and purged with N₂ for 3 times, and then the mixture was stirred at 130° C. for 2 h under N₂ atmosphere. On completion, the crude product (10S,17E)-14-[(2S)-1-{[tert-butyl(dimethyl)silyl]oxy}propan-2-yl]-12-ethyl-6,8,10-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (160 mg, in DMF) was used into the next step without further purification. LCMS: (M+1=821.4).

Step 3. To a solution of (10S,17E)-14-[(2S)-1-{[tert-butyl(dimethyl)silyl]oxy}propan-2-yl]-12-ethyl-6,8,10-trimethyl-2-(oxan-2-yl)-16-{[2-(trimethylsilyl)ethoxy]methoxy}-2,10,11,12,13,14-hexahydro-8H-5,3-(azenometheno)tripyrazolo[3,4-f:3′,4′-j:4″,3″-n][1,4]oxazacyclopentadecine (160 mg, 194 μmol, 1 eq) in DMF (2 mL) was added CsF (177 mg, 1.17 mmol, 6 eq). The mixture was stirred at 130° C. for 2 h. On completion, the mixture was diluted with water (10 mL) and extracted with ethyl acetate (10 mL×3). The combined organic phase was dried over anhydrous sodium sulfate, filtered and concentrated to give a residue. The residue was purified by column chromatography (SiO₂, Petroleum ether/Ethyl acetate=1:0 to 5:1) to give (2S)-2-[(10S,17E)-12-ethyl-6,8,10-trimethyl-2-(oxan-2-yl)-16-{[2-(trimethylsilyl)ethoxy]methoxy}-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (70.0 mg, 46.5 μmol, 24% yield, 47% purity) as a yellow solid. LCMS: (M+1=707.3).

Step 4. To a solution of (2S)-2-[(10S,17E)-12-ethyl-6,8,10-trimethyl-2-(oxan-2-yl)-16-{[2-(trimethylsilyl)ethoxy]methoxy}-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (60.0 mg, 84.8 mol, 1 eq) in TBAF (1 M in THF, 1 mL). The mixture was stirred at 70° C. for 16 h. On completion, the mixture was concentrated to give a residue, the residue was purified by column chromatography (SiO₂, DCM/MeOH=10:1 to 1:1) to give (10S,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (20.0 mg, 29.4 μmol, 35% yield, 85% purity) as a white solid. LCMS: (M+1=577.2).

Step 5. To a solution of (10S,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-ol (15.0 mg, 0.026 mmol, 1 eq) and 2-iodoacetonitrile (5.21 mg, 0.0312 mmol, 1.2 eq) in DMF (1 mL) was added K₂CO₃ (10.7 mg, 0.078 mmol, 3 eq). The mixture was stirred at 80° C. for 2 h. On completion, the mixture was quenched with water (10 mL) and extracted with EA (10 mL×3). The combined organic layers were washed with brine (15 mL×2), dried over Na₂SO₄, filtered and concentrated to give {[(10S,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (16.0 mg, 99% yield) as yellow solid. LCMS: (M+1=616.2).

Step 6. To a solution of {[(10S,17E)-12-ethyl-14-[(2S)-1-hydroxypropan-2-yl]-6,8,10-trimethyl-2-(oxan-2-yl)-2,10,11,12,13,14-hexahydro-8H-5,3-(azenometheno)tripyrazolo[3,4-f:3,4′-j:4″,3″-n][1,4]oxazacyclopentadecin-16-yl]oxy}acetonitrile (16.0 mg, 25.9 μmol, 1 eq) in DCM (0.5 mL) was added TFA (2.69 mmol, 0.2 mL, 103 eq). The mixture was stirred at 25° C. for 2 h. On completion, the mixture was filtered and concentrated to give a residue. The residue purified by prep-HPLC (column: Welch Ultimate C18 150*25 mm*5 um; mobile phase: [water(FA)-ACN]; gradient:12%-42% B over 10 min) to give Ex. 67 (2.83 mg, 4.75 μmol, 18.29% yield, 97% purity, FA) as a brown solid. LCMS: (M+1=532.1). ¹H NMR for Ex. 67 is shown in the below NMR table.

### Preparation of (2S)-2-[(10S,17E)-16-[(²H5)ethyloxy]-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 87)

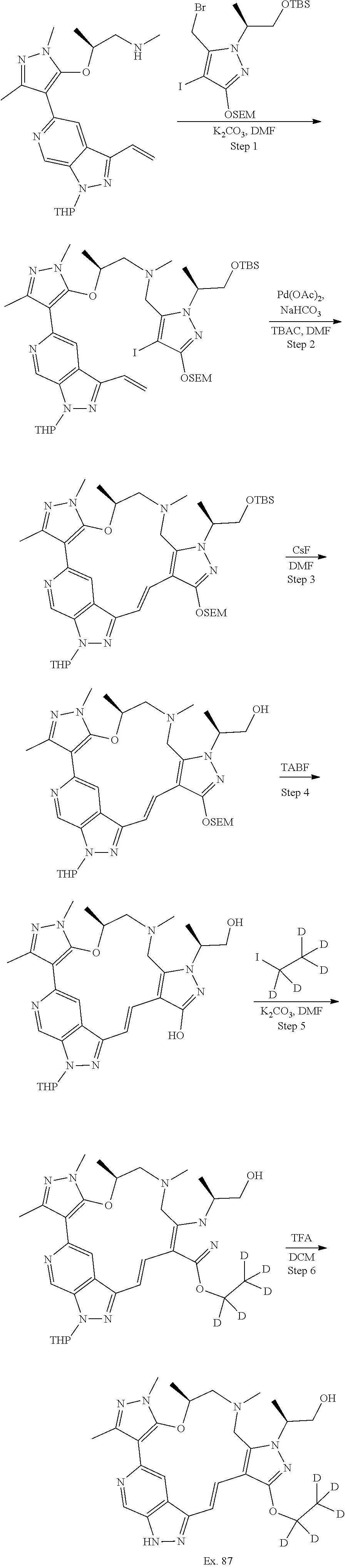

Ex. 87 was synthesized in the same manner as Ex. 67, using (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22, 2-[[5-(bromomethyl)-1-[(1S)-2-[tert-butyl(dimethyl)silyl]oxy-1-methyl-ethyl]-4-iodo-pyrazol-3-yl]oxymethoxy]ethyl-trimethyl-silane, which was prepared according to the method described in Ex. 52, and commercially available 1-iodoethane-1,1,2,2,2-d₅. ¹H NMR for Ex. 87 can be found in the below NMR table.

### Preparation of (2R)-2-[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 88)

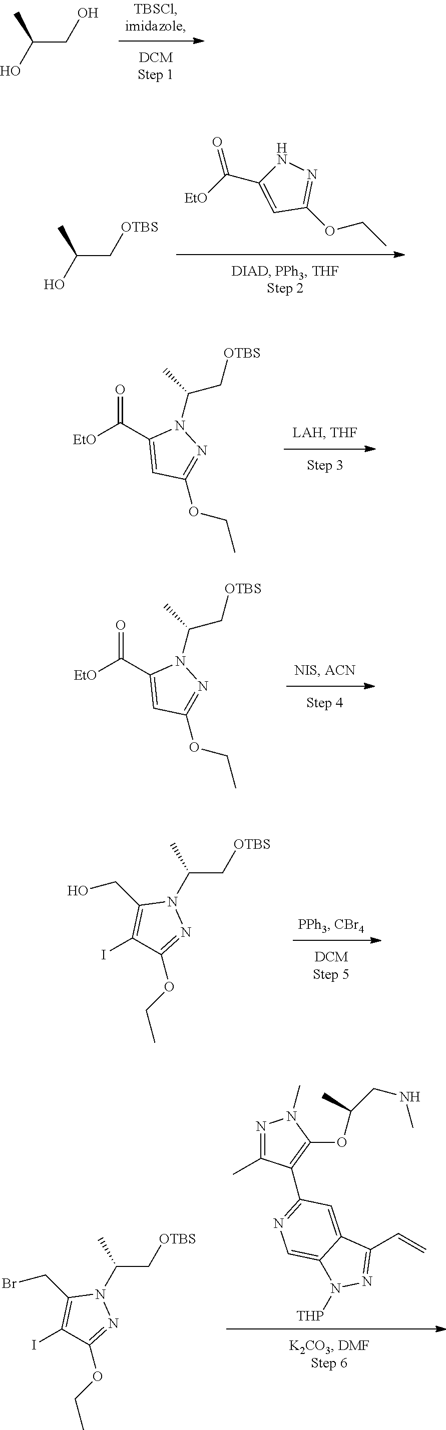

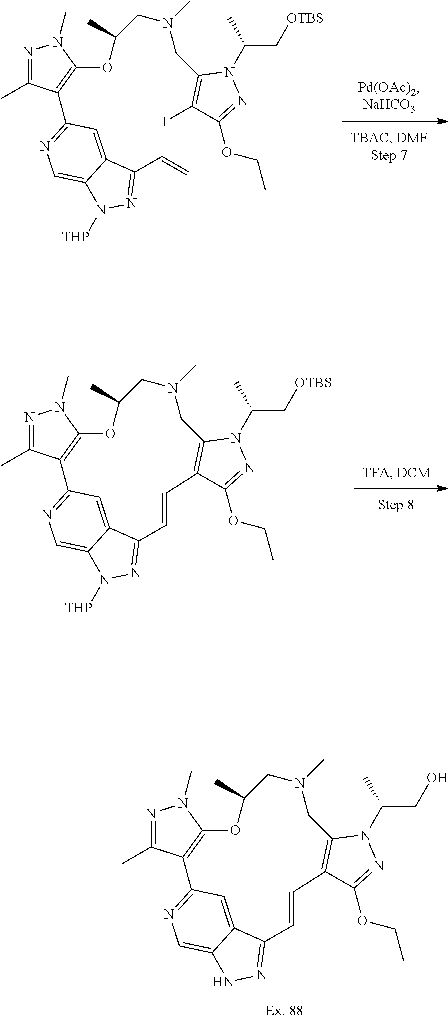

Step 1 through Step 5 were carried out in the same manner as in Ex. 19 using commercially available (2S)-propane-1,2-diol and commercially available ethyl 3-ethoxy-1H-pyrazole-5-carboxylate. Step 6 through Step 8 were carried out in the same manner as in Ex. 27 using (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22 and DMF in step 6. ¹H NMR for Ex. 88 can be found in the below NMR table.

### Preparation of (2R)-2-[(10R,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 89)

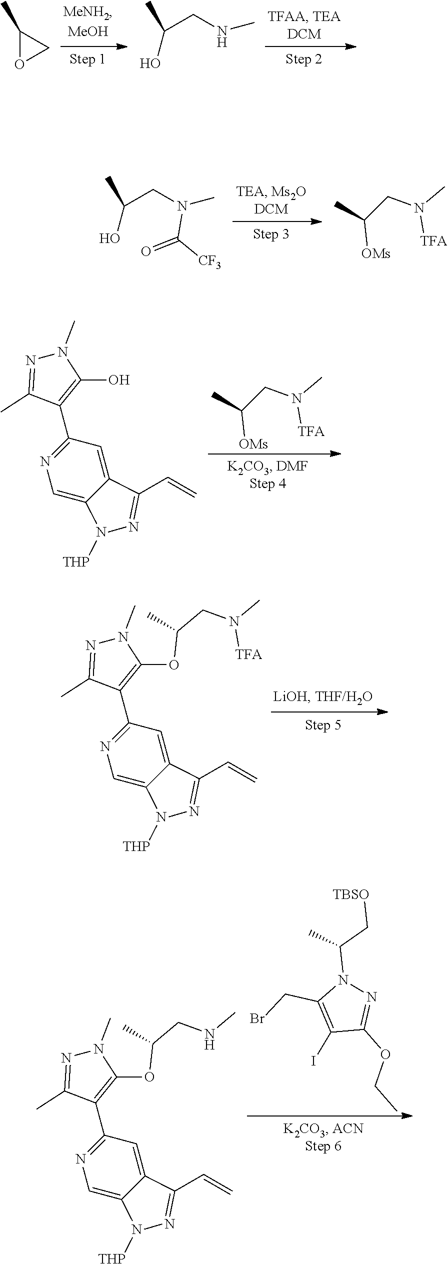

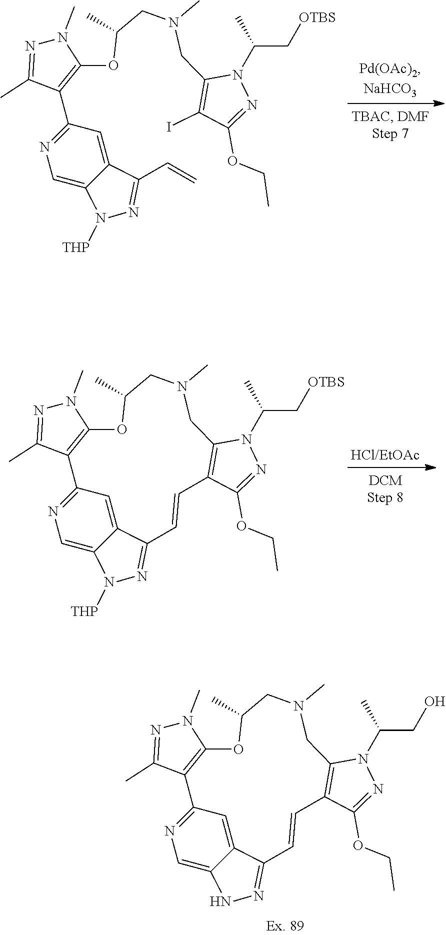

Step 1 through Step 5 were carried out in the same manner as in Ex. 36 using (2S)-2-methyloxirane and 2,5-dimethyl-4-(1-tetrahydropyran-2-yl-3-vinyl-pyrazolo[3,4-c]pyridin-5-yl)pyrazol-3-ol, which was prepared according to the method described in Ex. 1, and Step 6 and Step 7 were carried out in the same manner as in Ex. 27 using (R)-5-(bromomethyl)-1-(1-((tert-butyldimethylsilyl)oxy)propan-2-yl)-3-ethoxy-4-iodo-1H-pyrazole, which was prepared according to the method described in Ex. 88.

Step 8. To a solution of (10R,17E)-14-[(2R)-1-{[tert-butyl(dimethyl)silyl]oxy}propan-2-yl]-16-ethoxy-6,8,10,12-tetramethyl-2-(oxan-2-yl)-2,10,11,12,13,14-hexahydro-8H-5,3-(azenometheno)tripyrazolo[3,4-f3′,4′-j:4″,3″-n][1,4]oxazacyclopentadecine (10.0 mg, 14.1 μmol, 1 eq) in DCM (1 mL) was added HCl/EtOAc (4 M, 17.7 μL, 5 eq). The mixture was stirred at 25° C. for 1 hour. On completion, the reaction mixture was filtered, and the filtrate was concentrated under reduced pressure to remove solvent. The crude product was purified by reversed-phase column: Phenomenex luna C18 150*25 mm*10 um; mobile phase: [water(FA)-ACN]; gradient:13%-43% B to give Ex. 89 (7.03 mg, 97.83% yield) as a yellow solid. ³H NMR for Ex. 89 can be found in the below NMR table.

### Preparation of (2S)-1-[(10S,17E)-16-ethoxy-6,8,10,12,20-pentamethyl-2,8,10,11,12,13-hexahydro-14H-5,3-(azenometheno)tripyrazolo[3,4-f3′,4′-j:4″,3″-n][1,4]oxazacyclopentadecin-14-yl]propan-2-ol (Ex. 90)

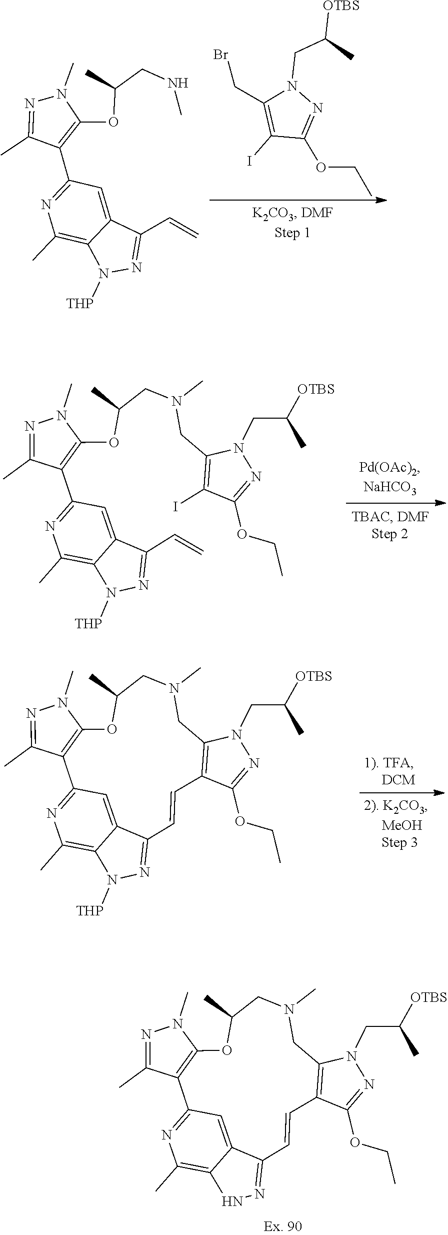

Ex. 90 was synthesized in the same manner as Ex. 27 using (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 47 and [(1S)-2-[5-(bromomethyl)-3-ethoxy-4-iodo-pyrazol-1-yl]-1-methyl-ethoxy]-tert-butyl-dimethyl-silane, which was prepared according to the method described in Ex. 86. ¹H NMR for Ex. 90 can be found in the below NMR table.

### Preparation of (2S)-2-[(10S,17E)-16-(ethylamino)-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 93)

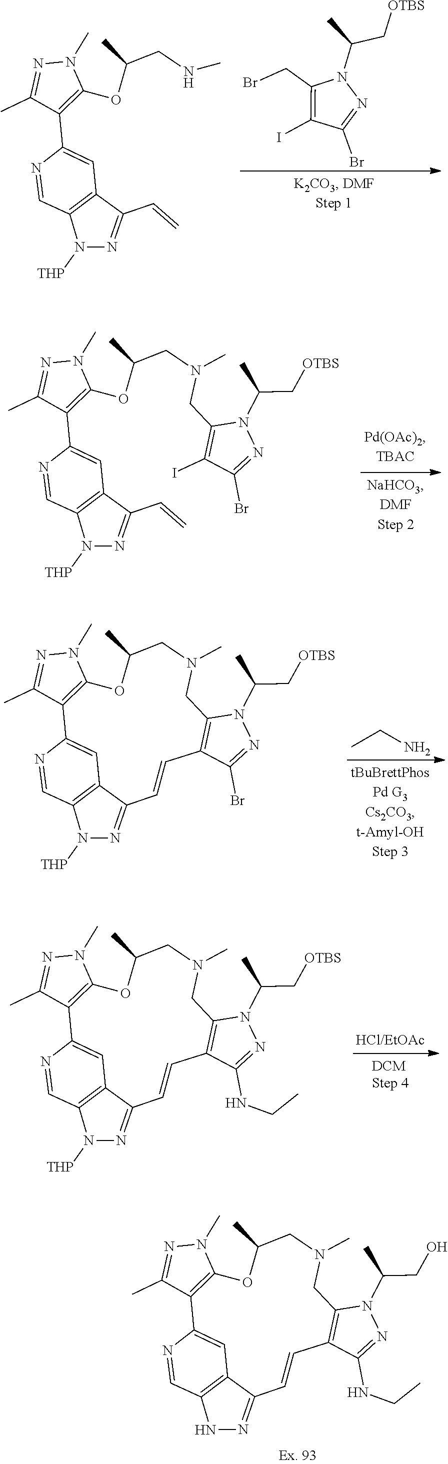

Step 1. To a solution of [(2S)-2-[3-bromo-5-(bromomethyl)-4-iodo-pyrazol-1-yl]propoxy]-tert-butyl-dimethyl-silane, which was prepared according to the method described in Ex. 48, (1.36 g, 1.26 mmol, 1 eq) and (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 22 (519 mg, 1.26 mmol, 1 eq) in DMF (18 mL) was added K₂CO₃ (349 mg, 2.53 mmol, 2 eq). The mixture was stirred at 80° C. for 30 min. On completion, the reaction mixture was partitioned between ethyl acetate (20 mL×3) and water (60 mL). The combined organic phase was washed with water (40 mL) and dried over anhydrous sodium sulfate, filtered and concentrated to give a residue. The residue was purified by column chromatography (SiO₂, Petroleum ether/THF=1:0 to 7:3) to give (2S)-N-[[5-bromo-2-[(1S)-2-[tert-butyl(dimethyl)silyl]oxy-1-methyl-ethyl]-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (570 mg, 571 μmol, 45% yield, 87% purity) as a colorless oil. ¹H NMR (400 MHz, CDCl₃)=9.23-9.11 (m, 1H), 8.02 (s, 1H), 7.93-7.85 (m, 1H), 7.08-6.97 (m, 1H), 6.10 (d, J=18.0 Hz, 1H), 5.80 (d, J=9.6 Hz, 1H), 5.64-5.54 (m, 1H), 4.73 (d, J=5.2 Hz, 1H), 4.31-4.17 (m, 1H), 4.14-4.02 (m, 1H), 3.84-3.74 (m, 2H), 3.72-3.67 (m, 3H), 3.54-3.48 (m, 1H), 3.41 (d, J=5.6 Hz, 1H), 2.96 (s, 2H), 2.89 (s, 2H), 2.67-2.49 (m, 2H), 2.24-1.97 (m, 6H), 1.72 (d, J=10.8 Hz, 2H), 1.34 (d, J=6.4 Hz, 3H), 1.13-1.05 (m, 3H), 0.81-0.70 (m, 9H), −0.04-0.12 (m, 3H), −0.15-0.34 (m, 3H). LCMS: (M+1=869.2).

Step 2. A mixture of (2S)-N-[[5-bromo-2-[(1S)-2-[tert-butyl(dimethyl)silyl]oxy-1-methyl-ethyl]-4-iodo-pyrazol-3-yl]methyl]-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine (510 mg, 511 μmol, 1 eq), TBAC (142 mg, 511 mol, 1 eq), NaHCO₃ (107 mg, 1.28 mmol, 2.5 eq) and Pd(OAc)₂ (23.0 mg, 102 mol, 0.2 eq) in DMF (5 mL) was degassed and purged with N₂ for 3 times, and then the mixture was stirred at 130° C. for 30 min under N₂ atmosphere. On completion, the reaction mixture was partitioned between ethyl acetate (25 mL×3) and water (100 mL). The combined organic phase was washed with water (40 mL), dried over anhydrous sodium sulfate, filtered, and concentrated. The residue was purified by column chromatography (SiO₂, Petroleum ether/THF=1:0 to 6:4) to give (10S,17E)-16-bromo-14-[(2S)-1-{[tert-butyl(dimethyl)silyl]oxy}propan-2-yl]-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,41oxazacyclopentadecine (300 mg, 365 μmol, 71% yield, 90% purity) as a brown oil. LCMS: (M+1=741.3).

Step 3. A mixture of the product from Step 2 (130 mg, 176 mol, 1 eq), ethanamine (2 M, 439 μL, 5 eq), Cs₂CO₃ (172 mg, 527 μmol, 3 eq), 1,3-bis[2,6-bis(1-propylbutyl)phenyl]-4,5-dichloro-2H-imidazol-1-ium-2-ide; 3-chloropyridine; dichloropalladium (tBuBrettPhos Pd G3) (17.1 mg, 17.6 mol, 0.1 eq) in t-Amyl-OH (1 mL) was degassed and purged with N₂ for 3 times, and then the mixture was stirred at 100° C. for 16 hr under N₂ atmosphere. On completion, the mixture was concentrated in vacuum to give a residue. The residue was purified by column chromatography (SiO₂, Petroleum ether/THF=1:0 to 2:1) to give (10S,17E)-14-|(2S)-1-{[tert-butyl(dimethyl)silyl]oxy}propan-2-yl]-N-ethyl-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-amine (75.0 mg, 107 μmol, 61% yield) as a colorless oil. LCMS: (M+1=704.5).

Step 4. To a solution of the product from Step 3 (60.0 mg, 85.2 μmol, 1 eq) in DCM (0.6 mL) was added HCl/EtOAc (4 M, 0.3 mL, 14.08 eq). The mixture was stirred at 25° C. for 1.5 hr. On completion, the mixture was concentrated in vacuum to give a residue. The residue was purified by prep-HPLC purification (column: Phenomenex luna C18 150*25 mm*10 um; mobile phase: [water(FA)-ACN]; gradient:4%-34% B over 10 min) to give Ex. 93. (12.51 mg, 29.03% yield) as a white solid. ¹H NMR for Ex. 93 can be found in the below NMR table.

### Preparation of (2S)-2-[(10S,17E)-16-(ethylamino)-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 94)

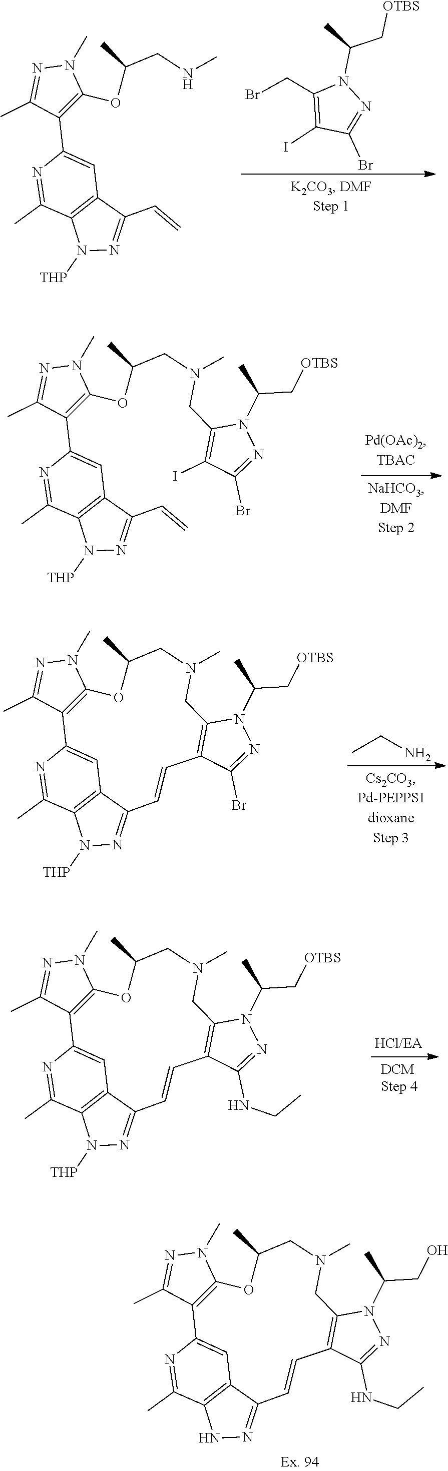

Ex. 94 was prepared according to the same method as described Ex. 93 substituting Pd-PEPPSI and dioxane for the catalyst and solvent respectively in step 3 and starting from (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine as prepared according to the method described in Ex. 47 instead of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine. ¹H NMR for Ex. 94 can be found in the below NMR table.

### Preparation of (2R)-2-[(10S,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol(Ex. 95)

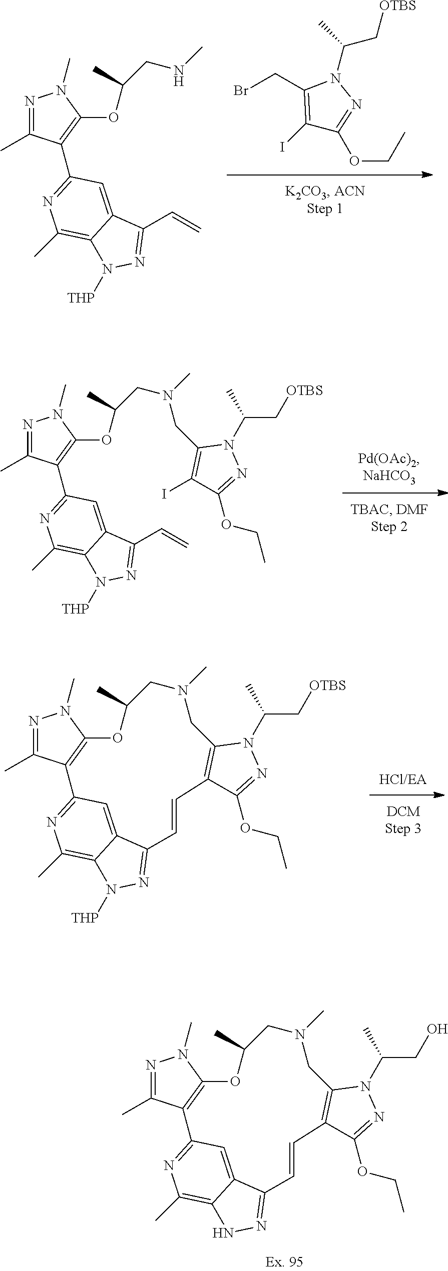

Ex. 95 was prepared according to the same method as described in steps 6-8 of Ex. 93 starting from (R)-5-(bromomethyl)-1-(1-((tert-butyldimethylsilyl)oxy)propan-2-yl)-3-ethoxy-4-iodo-1H-pyrazole, which was prepared according to the method described in Ex. 88, and (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine as prepared according to the method described in Ex. 47 instead of (2S)-2-[2,5-dimethyl-4-(1-tetrahydropyran-2-yl-3-vinyl-pyrazolo[3,4-c]pyridin-5-yl)pyrazol-3-yl]oxy-N-methyl-propan-1-amine. ¹H NMR for Ex. 95 can be found in the below NMR table.

### Preparation of (2S)-2-[(10S,17E)-16-ethoxy-20-methoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 96)

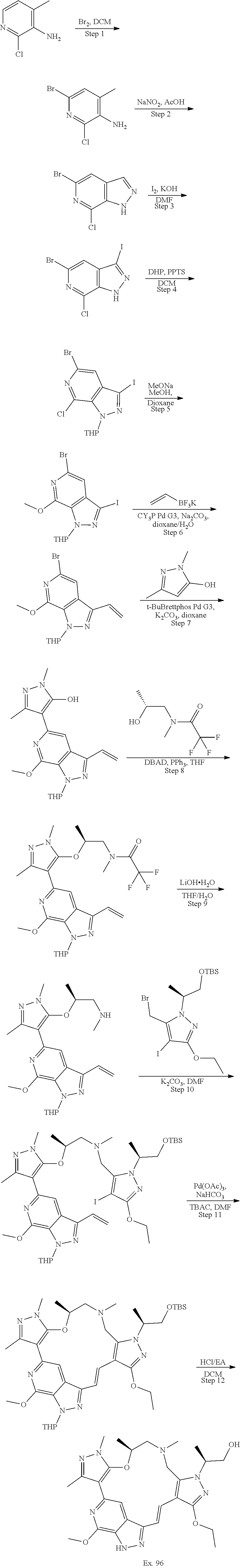

Step 1. To a solution of commercially available 2-chloro-4-methyl-pyridin-3-amine (10.0 g, 70.1 mmol, 1 eq) in DCM (100 mL) was added a solution of Br₂ (56.0 g, 350 mmol, 5 eq) in DCM (100 mL) dropwise at 0° C. The resulting mixture was stirred at 25° C. for 12 h. On completion, the reaction mixture was partitioned between DCM (200 mL×3) and water (200 mL). The combined organic phase was dried over anhydrous sodium sulfate, filtered, and concentrated. The residue was purified by column chromatography (SiO₂, PE:THF=2:1 to 2:1) to give 6-bromo-2-chloro-4-methyl-pyridin-3-amine (14.0 g, 63.2 mmol, 90% yield) as a white solid. LCMS: (M+1:222.8).

Step 2. To the mixture of 6-bromo-2-chloro-4-methyl-pyridin-3-amine (13.0 g, 58.7 mmol, 1 eq) in AcOH (130 mL) was added NaNO₂ (4.45 g, 64.5 mmol, 1.1 eq) at 0° C., the mixture was stirred at 25° C. for 12 h. On completion, the reaction mixture was partitioned between EA (150 mL×3) and water (150 mL). The combined organic phase was dried over anhydrous sodium sulfate, filtered, and concentrated to give a residue. The crude product was triturated with DCM (10 mL) at 25° C. for 5 mins to give 5-bromo-7-chloro-1H-pyrazolo[3,4-c]pyridine (4.50 g, 19.3 mmol, 32% yield) as a yellow solid. ¹H NMR (400 MHz, MeOD-d₄) δ=8.20 (br s, 1H), 7.95 (s, 1H).

Step 3. To a solution of 5-bromo-7-chloro-1H-pyrazolo[3,4-c]pyridine (4.00 g, 17.2 mmol, 1 eq) in DMF (40 mL) was added KOH (3.62 g, 64.5 mmol, 3.75 eq) and I₂ (8.73 g, 34.4 mmol, 2 eq) at 0° C. The mixture was stirred at 25° C. for 2 h. On completion, the reaction mixture was quenched by sat. Na₂SO₃ (30 mL). The mixture was partitioned between EA (40 mL×3) and water (40 mL). The combined organic phase was dried over anhydrous sodium sulfate, filtered, and concentrated. The residue was purified by column chromatography (SiO₂, PE:THF=1:1 to 1:1) to give 5-bromo-7-chloro-3-iodo-1H-pyrazolo[3,4-c]pyridine (6.00 g, 16.7 mmol, 97% yield) as a yellow solid. LCMS: (M+1:359.6).

Step 4. To a solution of 5-bromo-7-chloro-3-iodo-1H-pyrazolo[3,4-c]pyridine (5.70 g, 15.9 mmol, 1 eq) in DCM (180 mL) was added DHP (4.01 g, 47.7 mmol, 3 eq) and PPTS (799 mg, 3.18 mmol, 0.2 eq). The mixture was stirred at 30° C. for 4 h. On completion, the reaction mixture was partitioned between DCM (200 mL×3) and water (200 mL). The combined organic phase was dried over anhydrous sodium sulfate, filtered, and concentrated to give a residue. The crude product was triturated with MeOH (10 mL) at 25° C. for 5 mins to give 5-bromo-7-chloro-3-iodo-1-tetrahydropyran-2-yl-pyrazolo [3, 4-c]pyridine (5.41 g, 12.2 mmol, 76% yield) as a white solid. LCMS: (M+1:443.7).

Step 5. To a solution of 5-bromo-7-chloro-3-iodo-1-tetrahydropyran-2-yl-pyrazolo[3,4-c] pyridine (3.16 g, 7.14 mmol, 1 eq) in MeOH (15 mL) and dioxane (30 mL) was added CH₃ONa (463 mg, 8.57 mmol, 1.2 eq). The mixture was stirred at 40° C. for 12 h. On completion, the mixture was quenched with sat. NH₄Cl (100 mL) and extracted with EA (100 mL×3). The combined organic layers were washed with brine (100 mL×2), dried over Na₂SO₄, filtered, and concentrated. The residue was purified by column chromatography (SiO₂, PE:DCM=1:0 to 1:8) to give 5-bromo-3-iodo-7-methoxy-1-tetrahydropyran-2-yl-pyrazolo[3,4-c]pyridine (3.00 g, 6.85 mmol, 95% yield) as a yellow solid. LCMS: (M+1:437.8).

Step 6. To a solution of 5-bromo-3-iodo-7-methoxy-1-tetrahydropyran-2-yl-pyrazolo|3,4-c|pyridine (2.80 g, 6.39 mmol, 1 eq) in dioxane (56 mL) and H₂O (11.25 mL) was added Na₂CO₃ (2.03 g, 19.2 mmol, 3 eq), potassium vinyl trifluoroborate (856 mg, 6.39 mmol, 1 eq) and Cy₃P Pd G3 (415 mg, 639 μmol, 0.1 eq) under N₂, the mixture was stirred at 80° C. for 16 h. On completion, the mixture was quenched with water (150 mL) and extracted with 2-MeTHF (80 mL×3). The combined organic layers were washed with brine (100 mL×2), dried over Na₂SO₄, filtered, and concentrated to give a residue. The residue was purified by column chromatography (SiO₂,PE:THF=1:0 to 8:1) to give 5-bromo-7-methoxy-1-tetrahydropyran-2-yl-3-vinyl-pyrazolo[3,4-c]pyridine (1.28 g, 3.78 mmol, 59% yield) as a brown solid. LCMS: (M+1:337.9).

Step 7. To a solution of 5-bromo-7-methoxy-1-tetrahydropyran-2-yl-3-vinyl-pyrazolo[3,4-c]pyridine (1.11 g, 3.28 mmol, 1 eq) in dioxane (22 mL) was added commercially available 2,5-dimethylpyrazol-3-ol (478 mg, 4.27 mmol, 1.3 eq), K₂CO₃ (1.36 g, 9.85 mmol, 3 eq) and [2-(2-aminophenyl)phenyl]-methylsulfonyloxy-palladium; ditert-butyl-[3,6-dimethoxy-2-(2,4,6-triisopropylphenyl)phenyl]phosphane (280 mg, 328 μmol, 0.1 eq) under N₂. The mixture was stirred at 130° C. for 5 h under N₂ atmosphere. On completion, the mixture was filtered and concentrated. The residue was purified by column chromatography (SiO₂, Dichloromethane:Methanol=1:0 to 30:1) to give 4-(7-methoxy-1-tetrahydropyran-2-yl-3-vinyl-pyrazolo[3,4-c]pyridin-5-yl)-2,5-dimethyl-pyrazol-3-ol (730 mg, 1.98 mmol, 60% yield) as a yellow solid. LCMS: (M+1:370.1).

Step 8. A mixture of 4-(7-methoxy-1-tetrahydropyran-2-yl-3-vinyl-pyrazolo[3,4-c]pyridin-5-yl)-2,5-dimethyl-pyrazol-3-ol (700 mg, 1.89 mmol, 1 eq), commercially available 2,2,2-trifluoro-N-[(2R)-2-hydroxypropyl]-N-methyl-acetamide (701 mg, 3.79 mmol, 2 eq), PPh₃ (994 mg, 3.79 mmol, 2 eq) and DBAD (654 mg, 2.84 mmol, 1.5 eq) was degassed and purged with N₂ for 3 times, then THE (12 mL) was added. The mixture was stirred at 25° C. for 1 h. On completion, the mixture was concentrated, and the residue was purified by column chromatography (SiO₂,PE:THF=1:0 to 1:1) to give 2,2,2-trifluoro-N-[(2S)-2-[4-(7-methoxy-1-tetrahydropyran-2-yl-3-vinyl-pyrazolo[3,4-c]pyridin-5-yl)-2,5-dimethyl-pyrazol-3-yl]oxypropyl]-N-methyl-acetamide (850 mg, 1.58 mmol, 83% yield) as a yellow solid. LCMS: (M+1: 537.2).

Step 9. To a solution of 2,2,2-trifluoro-N-[(2S)-2-[4-(7-methoxy-1-tetrahydropyran-2-yl-3-vinyl-pyrazolo[3,4-c]pyridin-5-yl)-2,5-dimethyl-pyrazol-3-yl]oxypropyl]-N-methyl-acetamide (815 mg, 1.52 mmol, 1 eq) in THE (13 mL) and H₂O (2.6 mL) was added LiOH·H₂O (191 mg, 4.56 mmol, 3 eq). The mixture was stirred at 25° C. for 12 h. On completion, the mixture was quenched with water (40 mL) and extracted with EA (30 mL×3). The combined organic layers were washed with brine (30 mL×2), dried over Na₂SO₄. filtered and concentrated. The residue was purified by column chromatography (SiO₂, Dichloromethane:Methanol=1:0 to 8:1) to give (2S)-2-[4-(7-methoxy-1-tetrahydropyran-2-yl-3-vinyl-pyrazolo[3,4-c]pyridin-5-yl)-2,5-dimethyl-pyrazol-3-yl]oxy-N-methyl-propan-1-amine (68.0 mg, 154 μmol, 10% yield) as a yellow solid. LCMS: (M+1:441.2).

Step 10. To a solution of (2S)-2-[4-(7-methoxy-1-tetrahydropyran-2-yl-3-vinyl-pyrazolo[3,4-c]pyridin-5-yl)-2,5-dimethyl-pyrazol-3-yl]oxy-N-methyl-propan-1-amine (61.0 mg, 138 mol, 1 eq) in DMF (2 mL) was added [(2S)-2-[5-(bromomethyl)-3-ethoxy-4-iodo-pyrazol-1-yl]propoxy]-tert-butyl-dimethyl-silane, which was prepared according to the method described in Ex. 19, (69.7 mg, 138 mol, 1 eq) and K₂CO₃ (57.4 mg, 415 mol, 3 eq). The mixture was stirred at 60° C. for 1 h. On completion, the mixture was quenched with water (10 mL) and extracted with EA (10 mL×3). The combined organic layers were washed with brine (10 mL×2), dried over Na₂SO₄, filtered, and concentrated. The residue was purified by column chromatography (SiO₂, PE:THF=1:0 to 2:1) to give (2S)-N-[[2-[(1S)-2-[tert-butyl(dimethyl)silyl]oxy-1-methyl-ethyl]-5-ethoxy-4-iodo-pyrazol-3-yl]methyl]-2-[4-(7-methoxy-1-tetrahydropyran-2-yl-3-vinyl-pyrazolo[3,4-c]pyridin-5-yl)-2,5-dimethyl-pyrazol-3-yl]oxy-N-methyl-propan-1-amine (76.0 mg, 88.0 mol, 63% yield) as a yellow solid. LCMS: (M+1:863.3).

Step 11. A mixture of (2S)-N-[[2-[(1S)-2-[tert-butyl(dimethyl)silyl]oxy-1-methyl-ethyl]-5-ethoxy-4-iodo-pyrazol-3-yl]methyl]-2-[4-(7-methoxy-1-tetrahydropyran-2-yl-3-vinyl-pyrazolo[3,4-c]pyridin-5-yl)-2,5-dimethyl-pyrazol-3-yl]oxy-N-methyl-propan-1-amine (66.0 mg, 76.5 μmol, 1 eq), NaHCO₃ (16.1 mg, 191 μmol, 2.5 eq), TBAC (21.3 mg, 76.5 μmol, 1 eq) and Pd(OAc)₂ (3.43 mg, 15.3 μmol, 0.2 eq) in DMF (3.5 mL) was degassed and purged with N₂ for 3 times, and then the mixture was stirred at 130° C. for 1 h under N₂ atmosphere. On completion, the mixture was quenched with water (10 mL) and extracted with EA (10 mL×3). The combined organic layers were washed with brine (10 mL×2), dried over Na₂SO₄, filtered, and concentrated. The residue was purified by column chromatography (SiO₂, Petroleum ether/Ethyl acetate=1:0 to 1:1) to give (10S,17E)-14-[(2S)-1-{[tert-butyl(dimethyl)silyl]oxy}propan-2-yl]-16-ethoxy-20-methoxy-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (54.0 mg, 73.5 mol, 96% yield) as a yellow solid. LCMS: (M+1:735.4).

Step 12. To a solution of the product from Step 11 (5.00 mg, 6.80 mol, 1 eq) in DCM (0.5 mL) was added HCV/EtOAc (4 M, 0.1 mL, 58.8 eq). The mixture was stirred at 25° C. for 1 h. On completion, the mixture was concentrated to give Ex. 96 (1.31 mg, 32.53% yield) as an off-white solid. ¹H NMR for Ex. 96 can be found in the below NMR table.

### Preparation of (2S)-2-1(10R,17E)-16-ethoxy-6,8,10,12,20-pentamethyl-2,8,10,11,12,13-hexahydro-14H-5,3-(azenometheno)tripyrazolo[3,4-f3′,4′-j:4″,3″-n][1,4]oxazacyclopentadecin-14-yl]propan-1-ol (Ex. 98)

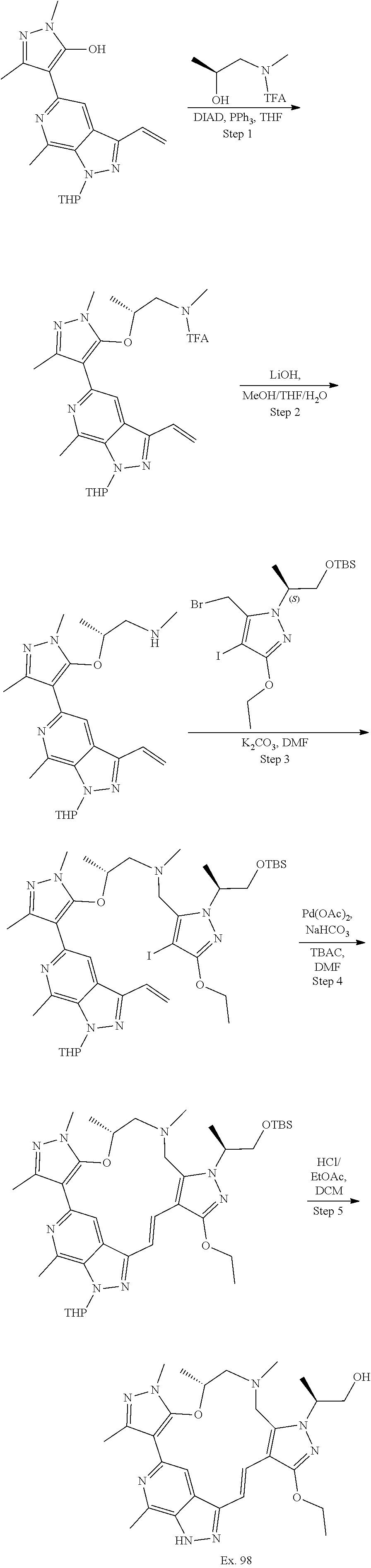

Steps 1 and 2 of Ex. 98 were carried out in the same manner as steps 2 and 3 of Ex. 23 substituting DIAD for DBAD and using 2,5-dimethyl-4-(7-methyl-1-tetrahydropyran-2-yl-3-vinyl-pyrazolo[3,4-c]pyridin-5-yl)pyrazol-3-ol which was prepared according to the method described in Ex. 47, commercially available 2,2,2-trifluoro-N-[(2S)-2-hydroxypropyl]-N-methyl-acetamide, and DIAD. Steps 3, 4, and 5 were carried out in the same manner as steps 6, 7, and 8 of Ex. 89 using [(2S)-2-[5-(bromomethyl)-3-ethoxy-4-iodo-pyrazol-1-yl]propoxy]-tert-butyl-dimethyl-silane, which was prepared according to the method described in Ex. 19, and DMF in step 3. ¹H NMR for Ex. 98 can be found in the below NMR table.

### Preparation of (10S,17E)-16-ethoxy-14-[(2S)-1-hydroxypropan-2-yl]-6,8,10,12-tetramethyl-2,10,11,12,13,14-hexahydro-8H-5,3-(azenometheno)tripyrazolo[3,4-f:3′,4′-j:4″,3″-n][1,4]oxazacyclopentadecin-20-ol (Ex. 99)

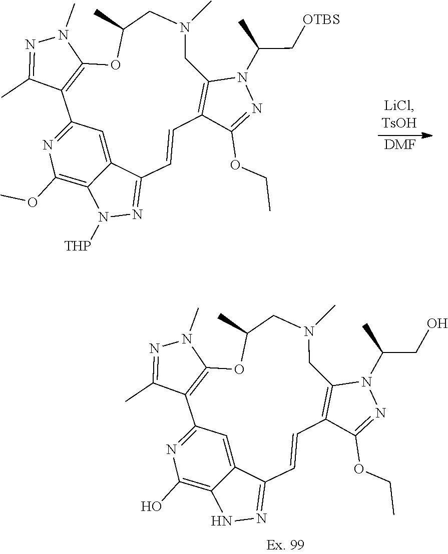

To a solution of (10S,17E)-14-[(2S)-1-f [tert-butyl(dimethyl)silyl]oxy}propan-2-yl]-16-ethoxy-20-methoxy-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine, which was prepared according to the method described in Ex. 96, (20.0 mg, 27.2 mol, 1 eq) in DMF (1.6 mL) was added TsOH (23.4 mg, 136 mol, 5 eq) and LiCl (5.77 mg, 136 μmol, 5 eq). The mixture was stirred at 120° C. for 1 h. On completion, the mixture was quenched with water (10 mL) and extracted with DCM:MeOH=10:1 (15 mL×3). The combined organic layers were dried over Na₂SO₄. filtered, and concentrated. The residue was purified by prep-HPLC (column: Waters xbridge 150*25 mm 10 um; mobile phase: [water(NH₄HCO₃)-ACN]; gradient:20%-50% B over 10 min) to give Ex. 99 (1.59 mg, 10.9% yield) as an off-white solid. ¹H NMR for Ex. 99 can be found in the below NMR table.

### Preparation of (2R)-2-1(10R,17E)-16-ethoxy-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 100)

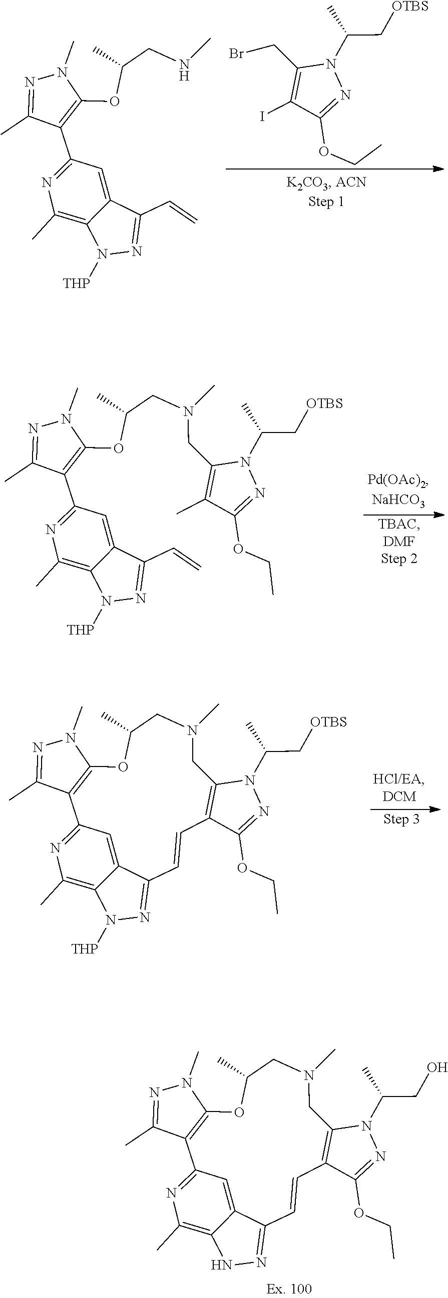

Ex. 100 was prepared according to the method described in steps 6-8 of Ex. 89 using 2,5-dimethyl-4-(7-methyl-1-tetrahydropyran-2-yl-3-vinyl-pyrazolo[3,4-c]pyridin-5-yl)pyrazol-3-ol which was prepared according to the method described in Ex. 47 and (R)-5-(bromomethyl)-1-(1-((tert-butyldimethylsilyl)oxy)propan-2-yl)-3-ethoxy-4-iodo-1H-pyrazole, which was prepared according to the method described in Ex. 88. ¹H NMR for Ex. 100 can be found in the below NMR table.

### Preparation of (2S)-2-[(10S,17E)-6,8,10,12,20-pentamethyl-16-(methylamino)-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 101) and (2S)-2-[(10S,17E)-16-[ethyl(methyl)amino]-6,8,10,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 102)

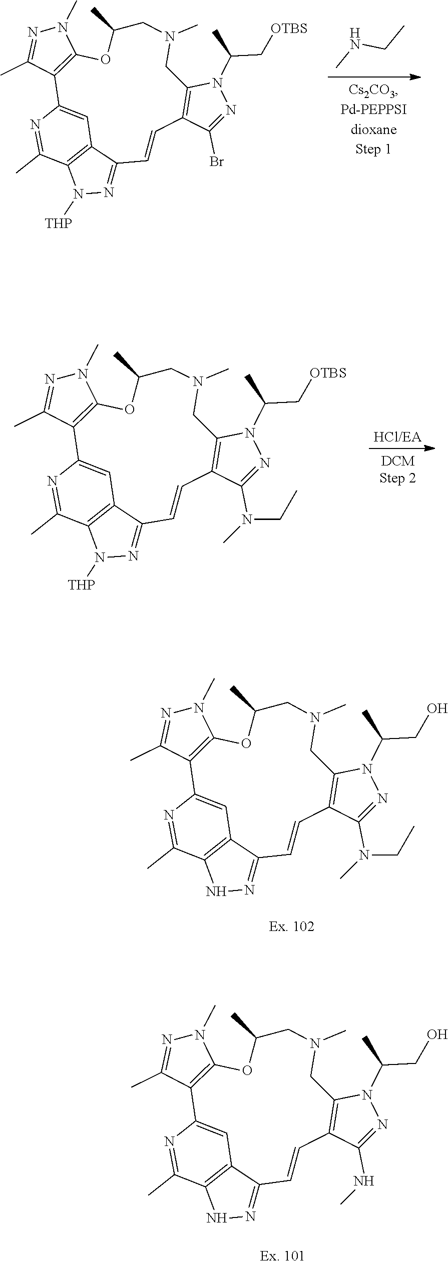

Step 1. A mixture of the above intermediate from Ex. 94 ((500 mg, 663 mol, 1 eq), N-methylethanamine (392 mg, 6.63 mmol, 570 μL, 10 eq), Cs₂CO₃ (648 mg, 1.99 mmol, 3 eq), and 1,3-bis[2,6-bis(1-propylbutyl)phenyl]-4,5-dichloro-2H-imidazol-1-ium-2-ide; 3-chloropyridine; dichloropalladium (Pd-PEPPSI) (64.5 mg, 66.3 mol, 0.1 eq) in dioxane (5 mL) was degassed and purged with N₂ for 3 times, and then stirred at 100° C. for 24 hr under N₂ atmosphere. On completion, the reaction mixture was concentrated under reduced pressure to remove solvent. The residue was purified by column chromatography (SiO₂, Petroleum ether/Ethyl acetate=1/0 to 0/1) to give (10S,17E)-14-[(2S)-1-{[tert-butyl(dimethyl)silyl]oxy}propan-2-yl]-N-ethyl-N,6,8,10,12,20-hexamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-amine (290 mg, 396 mol, 60% yield) as yellow solid. LCMS: (M+1=732.4).

Step 2. To a solution of the intermediate obtained from Step 1 (260 mg, 355 μmol, 1 eq) in DCM (3 mL) was added HCl/EA (4 M, 1 mL, 11.26 eq) at 0° C. The mixture was stirred at 25° C. for 2 hr. On completion, the reaction mixture was concentrated under reduced pressure to remove solvent. The residue was purified by prep-HPLC (column: Phenomenex luna C18 150*25 mm*10 um; mobile phase: [water(FA)-ACN]; gradient:9%-39% B over 10 min). The residue was further purified by prep-HPLC (column: Waters xbridge 150*25 mm 10 um; mobile phase: [water(NH₄HCO₃)-ACN]; gradient:31%-61% B over 15 min) to give Ex. 101 (3.13 mg, 5.76 mol, 1.62% yield, 93% purity) as a white solid and Ex. 102 (6.48 mg, 3.18% yield) as a white solid. ¹H NMR for Ex. 101 and Ex. 102 are shown in the below NMR table.

### Preparation of (10S,17E)-6,8,10,12,14,16,20-heptamethyl-2,10,11,12,13,14-hexahydro-8H-5,3-(azenometheno)tripyrazolo[3,4-f:3′,4′-j:4″,3″-n][1,4]oxazacyclopentadecine (Ex. 103)

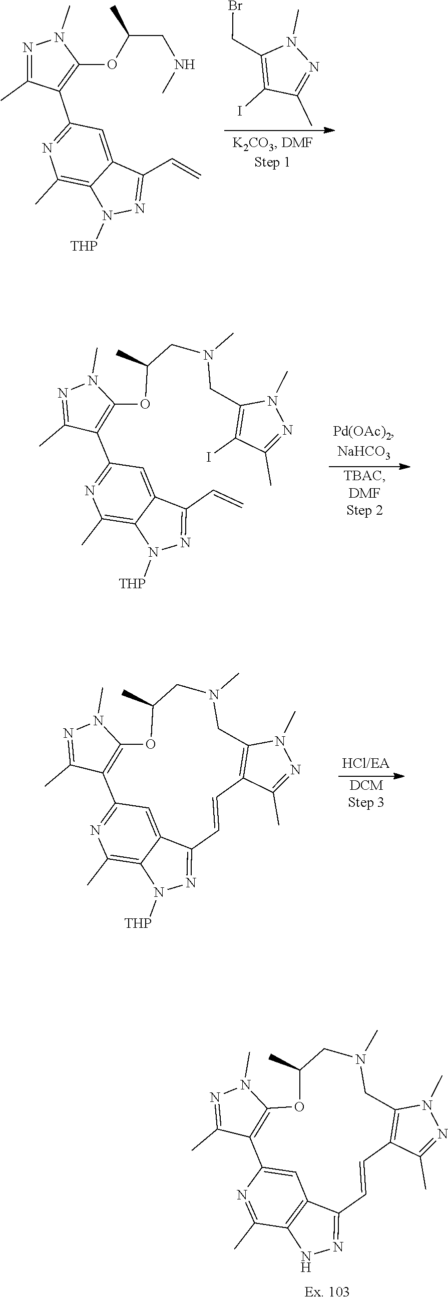

Step 1. To a solution of (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-methyl-propan-1-amine, which was prepared according to the method described in Ex. 47 (500 mg, 1.18 mmol, 1 eq) in DMF (8.7 mL) and commercially available 5-(bromomethyl)-4-iodo-1,3-dimethyl-pyrazole (370 mg, 1.18 mmol, 1 eq) was added K₂CO₃ (488 mg, 3.53 mmol, 3 eq). The mixture was stirred at 25° C. for 2 hr. On completion, the mixture was quenched with water (20 mL) and extracted with ethyl acetate (25 mL×3). The combined organic phase was dried over anhydrous sodium sulfate, filtered, and concentrated. The residue was purified by column chromatography (SiO₂, DCM/MeOH=1:1) to give (2S)-2-[2,5-dimethyl-4-(7-methyl-1-tetrahydropyran-2-yl-3-vinyl-pyrazolo[3,4-c]pyridin-5-yl)pyrazol-3-yl]oxy-N-[(4-iodo-2,5-dimethyl-pyrazol-3-yl)methyl]-N-methyl-propan-1-amine (600 mg, 874 μmol, 74% yield) as a yellow oil. LCMS: (M+1=659.2).

Step 2. A mixture of the product from Step 4 (550 mg, 835 mol, 1 eq), TBAC (232 mg, 835 mol, 1 eq), NaHCO₃ (175 mg, 2.09 mmol, 2.5 eq) and Pd(OAc)₂ (37.5 mg, 167 μmol, 0.2 eq) in DMF (6 mL) was degassed and purged with N₂ for 3 times, and then the mixture was stirred at 140° C. for 2 hr under N₂ atmosphere. On completion, the mixture was quenched with water (15 mL) and extracted with ethyl acetate (15 mL×3). The combined organic phase was dried over anhydrous sodium sulfate, filtered and concentrated to give (10S,17E)-6,8,10,12,14,16,20-heptamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (400 mg, 754 μmol, 90% yield, crude) as a yellow oil. LCMS: (M+1=531.3).

Step 3. To a solution of the product from Step 5 (350 mg, 659 μmol, 1 eq) in DCM (4 mL) was added HCl/EA (659 μmol, 2 mL, 1 eq). The mixture was stirred at 25° C. for 2 hr. On completion, the mixture was filtered and concentrated. The residue was purified by prep-HPLC (column: Phenomenex luna C18 150*25 mm*10 um; mobile phase: [water(FA)-ACN]; gradient:0%-28% B over 10 min) to give Ex. 103 (66.38 mg, 19.15% yield) as a yellow solid. ¹H NMR for Ex. 103 can be found in the below NMR table.

### Preparation of (2S)-2-[(10S,17E)-16-[ethyl(methyl)amino]-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 105)

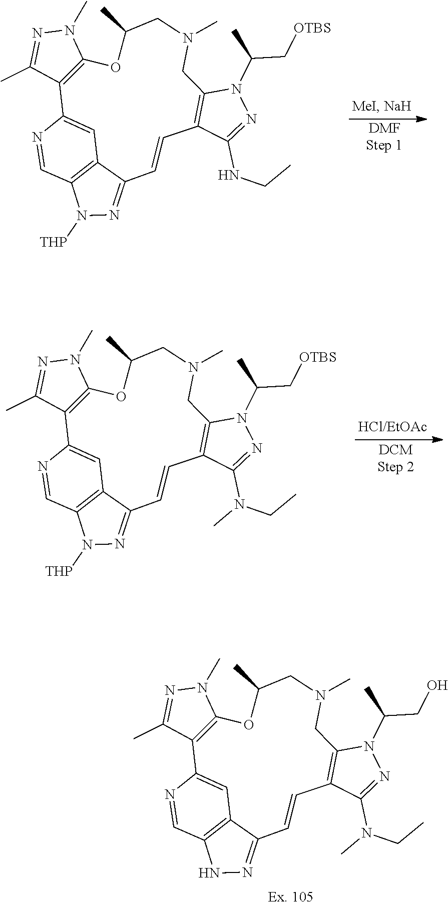

Step 1. To a solution of (10S,17E)-14-[(2S)-1-{[tert-butyl(dimethyl)silyl]oxy}propan-2-yl]-N-ethyl-6,8,10,12-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-amine, which was prepared according to the method described in Ex. 93, (10.0 mg, 0.0142 mmol, 1 eq) in DMF (0.6 mL) was added NaH (1.25 mg, 0.0313 mmol, 60% purity, 2.2 eq) at 0° C. and stirred at 25° C. for 30 min. Then MeI (2.42 mg, 0.0171 mmol, 1.2 eq) was added and stirred at 25° C. for 30 min. On completion, the mixture was poured into a saturated solution of NH₄Cl (6 mL), then extracted with EA (10 mL×3 and water (20 mL). The combined organic phase was washed with brine (20 mL), dried over anhydrous sodium sulfate, filtered, and concentrated. The crude was obtained as a black solid (10 mg) and used directly to the next step without further purification. LCMS: (M+1=718.6).

Step 2. To a solution of the crude product from Step 1 (8.00 mg, 3.34 mol, 1 eq) in DCM (0.2 mL) was added HCl/EtOAc (2 M, 1.67 μL, 1 eq). The mixture was stirred at 25° C. for 1.5 hr. On completion, the mixture was concentrated in vacuum. The residue was purified by prep-HPLC purification (column: Waters Xbridge 150*25 mm*Sum; mobile phase: [water(NH₃H₂O)-ACN]; gradient:26%-56% B over 10 min) to give Ex. 105 (0.40 mg, 6.49e⁻¹ μmol, 19.41% yield, 84.13% purity) as a white solid. ¹H NMR for Ex. 105 can be found in the below NMR table.

### Preparation of (2S)-2-[(10S,17E)-6,16-diethoxy-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 120)

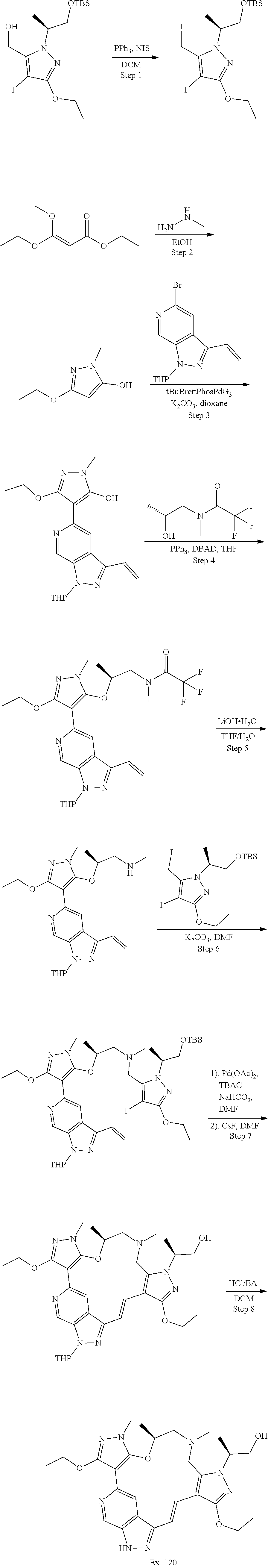

Step 1. A solution of [2-[(1S)-2-[tert-butyl(dimethyl)silyl]oxy-1-methyl-ethyl]-5-ethoxy-4-iodo-pyrazol-3-yl]methanol, which was prepared according to the method described in Ex. 19, (1.00 kg, 2.27 mol, 1 eq) and NIS (1.02 kg, 4.54 mol, 2 eq) in DCM (7 L) was degassed and purged with N₂ for 3 times, and then a solution of PPh₃ (893 g, 3.41 mol, 1.5 eq) in DCM (800 mL) was slowly added to the reaction solution at 0° C. The mixture was stirred at 25° C. for 1 h under N₂ atmosphere. On completion, the mixture was poured into saturated sodium sulfite aqueous solution (7000 mL), and the aqueous phase was extracted with DCM (2000 mL×3). The combined organic phase was washed with brine (2000 mL×2), dried over anhydrous Na₂SO₄, filtered, and concentrated under vacuum. The residue was purified by silica gel chromatography (column height: 250 mm, diameter: 100 mm, 100-200 mesh silica gel, Petroleum ether/Ethyl acetate=I/O, 10/1) to give tert-butyl-[(2S)-2-[3-ethoxy-4-iodo-5-(iodomethyl)pyrazol-1-yl]propoxy]-dimethyl-silane (950 g, 71% yield) as a yellow oil. LCMS: (M+1:551.1).

Step 2. To a solution of commercially available ethyl 3,3-diethoxyprop-2-enoate (1.00 g, 5.31 mmol, 1.00 eq) in EtOH (10 mL) was added methylhydrazine (734 mg, 6.38 mmol, 0.840 mL, 1.20 eq) at 25° C. The mixture was stirred at 80° C. for 16 hr. On completion, the mixture was filtered, and the organic phase concentrated. The residue was purified by column chromatography (SiO₂, Petroleum ether/THF=1/0 to 1/1) to give 5-ethoxy-2-methyl-pyrazol-3-ol (388 mg, 2.48 mmol, 47% yield, 91% purity) as a red solid. ¹H NMR (400 MHz, MeOD-d₄) δ=4.26-4.19 (m, 2H), 4.19-4.14 (m, 1H), 3.39 (s, 1H), 3.17 (s, 2H), 1.39-1.31 (m, 3H), 1.29-1.23 (m, 1H).

Step 3. A mixture of 5-ethoxy-2-methyl-pyrazol-3-ol (360 mg, 2.53 mmol, 1.00 eq), 5-bromo-1-tetrahydropyran-2-yl-3-vinyl-pyrazolo[3,4-c]pyridine, which was prepared according to the method described in Ex. 1, (937 mg, 3.04 mmol, 1.20 eq), K₂CO₃ (700 mg, 5.06 mmol, 2.00 eq), and [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (216 mg, 253 mol, 0.10 eq) in dioxane (10 mL) was degassed and purged with N₂ for 3 times, and then the mixture was stirred at 110° C. for 2 hr under N₂ atmosphere. On completion, the mixture was filtered, and the filtrate was concentrated. The residue was purified by column chromatography (SiO₂, Petroleum ether/THF=1/0 to 1/1) to give 5-ethoxy-2-methyl-4-(1-tetrahydropyran-2-yl-3-vinyl-pyrazolo [3,4-c]pyridin-5-yl)pyrazol-3-ol (385 mg, 39% yield) as a brown solid. LCMS: (M+1:370.0).

Step 4. A mixture of 5-ethoxy-2-methyl-4-(1-tetrahydropyran-2-yl-3-vinyl-pyrazolo[3,4-c]pyridin-5-yl)pyrazol-3-ol (360 mg, 0.975 mmol, 1.00 eq), commercially available 2,2,2-trifluoro-N-[(2R)-2-hydroxypropyl]-N-methyl-acetamide (361 mg, 1.95 mmol, 2.00 eq), DBAD (337 mg, 1.46 mmol, 1.50 eq), and PPh₃ (511 mg, 1.95 mmol, 2.00 eq) in THF (5 mL) was degassed and purged with N₂ for 3 times, and then the mixture was stirred at 25° C. for 2 hr under N₂ atmosphere. On completion, the mixture was concentrated in vacuum. The residue was purified by column chromatography (SiO₂, Petroleum ether/THF=1/0 to 1/1) to give N-[(2S)-2-[5-ethoxy-2-methyl-4-(1-tetrahydropyran-2-yl-3-vinyl-pyrazolo[3,4-c]pyridin-5-yl)pyrazol-3-yl]oxypropyl]-2,2,2-trifluoro-N-methyl-acetamide (145 mg) as a colorless oil. ¹H NMR (400 MHz, CDCl₃) δ=9.03-8.94 (m, 1H), 8.37-8.29 (m, 1H), 7.05 (dd, J=11.6, 18.0 Hz, 1H), 6.13 (d, J=18.0 Hz, 1H), 5.76 (d, J=9.2 Hz, 1H), 5.59 (d, J=11.6 Hz, 1H), 5.20-5.01 (m, 1H), 4.42-4.30 (m, 2H), 4.08 (d, J=9.6 Hz, 1H), 3.95 (d, J=13.6 Hz, 1H), 3.84-3.73 (m, 2H), 3.63-3.59 (m, 3H), 3.37-3.28 (m, 3H), 2.17-2.07 (m, 3H), 1.85-1.73 (m, 3H), 1.46-1.42 (m, 3H), 1.21-1.14 (m, 3H). LCMS (M+1=537.3).

Step 5. To a solution of N-[(2S)-2-[5-ethoxy-2-methyl-4-(1-tetrahydropyran-2-yl-3-vinyl-pyrazolo[3,4-c]pyridin-5-yl)pyrazol-3-yl]oxypropyl]-2,2,2-trifluoro-N-methyl-acetamide (140 mg, 0.261 mmol, 1.00 eq) in THE (2 mL) and H₂O (0.4 mL) was added LiOH·H2O (33.0 mg, 0.783 mmol, 3.00 eq). The mixture was stirred at 25° C. for 1 hr. On completion, the mixture was quenched with water (30 mL) and extracted with ethyl acetate (15 mL×3). The combined organic phase was washed with brine (20 mL×3), dried over anhydrous sodium sulfate, filtered and concentrated in vacuum to give (2S)-2-[5-ethoxy-2-methyl-4-(1-tetrahydropyran-2-yl-3-vinyl-pyrazolo[3,4-c]pyridin-5-yl)pyrazol-3-yl]oxy-N-methyl-propan-1-amine (113 mg, crude) as a colorless oil. LCMS: (M+1: 441.3).

Step 6. To a solution of the crude product from Step 4 (113 mg, 0.257 mmol, 1.00 eq) in DMF (2 mL) was added K₂CO₃ (106 mg, 0.770 mmol, 3.00 eq) and tert-butyl-[(2S)-2-[3-ethoxy-4-iodo-5-(iodomethyl)pyrazol-1-yl]propoxy]-dimethyl-silane (155 mg, 0.282 mmol, 1.10 eq). The mixture was stirred at 80° C. for 2 hr. On completion, the mixture was quenched with water (15 mL) and extracted with ethyl acetate (5 mL×3). The combined organic phase was washed with brine (5 mL×3), dried over anhydrous sodium sulfate, filtered, and concentrated in vacuum. The residue was purified by column chromatography (SiO₂, Petroleum ether/EA=1/0 to 1/1) to give (2S)-N-[[2-[(1S)-2-[tert-butyl(dimethyl)silyl]oxy-1-methyl-ethyl]-5-ethoxy-4-iodo-pyrazol-3-yl]methyl]-2-[5-ethoxy-2-methyl-4-(1-tetrahydropyran-2-yl-3-vinyl-pyrazolo[3,4-c]pyridin-5-yl)pyrazol-3-yl]oxy-N-methyl-propan-1-amine (171 mg, 66% yield) as a yellow oil. LCMS: (M+1: 863.4).

Step 7. A mixture of the product from Step 5 (160 mg, 0.185 mmol, 1.00 eq), Pd(OAc)₂ (5.00 mg, 0.0185 mmol, 0.10 eq), TBAC (52.0 mg, 0.185 mmol, 1.00 eq) and NaHCO₃ (47.0 mg, 0.556 mmol, 3.00 eq) in DMF (2.5 mL) was degassed and purged with N₂ for 3 times, and then the mixture was stirred at 130° C. for 1 hr under N₂ atmosphere. After cooling down, CsF (140 mg, 0.918 mmol, 5.00 eq) was added to the reaction mixture, and stirred at 25° C. for 16 hrs. On completion, the mixture was diluted with water (30 mL) and extracted with 2-MeTHF (15 mL×3). The combined organic phase was washed with brine (20 mL×3), dried over anhydrous sodium sulfate, filtered and concentrated in vacuum. The residue was purified by column chromatography (SiO₂, DCM/MeOH=1/0 to 10/1) to give (2S)-2-[(10S,17E)-6,16-diethoxy-8,10,12-trimethyl-2-(oxan-2-yl)-2,8,10,11,12,13-hexahydro-14H-5,3-(azenometheno)tripyrazolo[3,4-f3′,4′-j:4″,3″-n][1,4]oxazacyclopentadecin-14-yl]propan-1-ol (48.0 mg, 42.% yield) as a red solid. ¹H NMR (400 MHz, CDCl₃) δ=9.14 (d, J=3.6 Hz, 1H), 8.29 (s, 1H), 8.17 (d, J=16.8 Hz, 1H), 7.35 (dd, J=4.8, 17.6 Hz, 1H), 5.81-5.71 (m, 1H), 4.89-4.75 (m, 1H), 4.44-4.29 (m, 6H), 4.18-4.00 (m, 2H), 3.93 (s, 2H), 3.87 -3.73 (m, 4H), 3.64 (s, 3H), 2.79 (d, J=4.8 Hz, 2H), 2.58-2.47 (m, 1H), 2.43 (s, 3H), 2.21-2.04 (m, 3H), 1.81-1.74 (m, 2H), 1.48 (s, 6H), 1.44 (d, J=6.8 Hz, 3H).

Step 8. To a solution of the product from Step 7 (40.0 mg, 0.0644 mmol, 1.00 eq) in DCM (4 mL) was added HCl/EtOAc (2 M, 0.800 mL, 24.8 eq). The mixture was stirred at 25° C. for 1 hr. On completion, the mixture was concentrated in vacuum, and the residue was purified by prep-HPLC (column: Phenomenex luna C18 150*25 mm*10 um; mobile phase: [water(FA)-ACN]; gradient:20%-50% B over 10 min) to give Ex. 120 (5.64 mg) as a yellow solid. ¹H NMR for Ex. 120 can be found in the below NMR table.

### Preparation of (2S)-2-[(10S,17E)-16-ethoxy-6-(methoxymethyl)-8,10,12-trimethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 130)

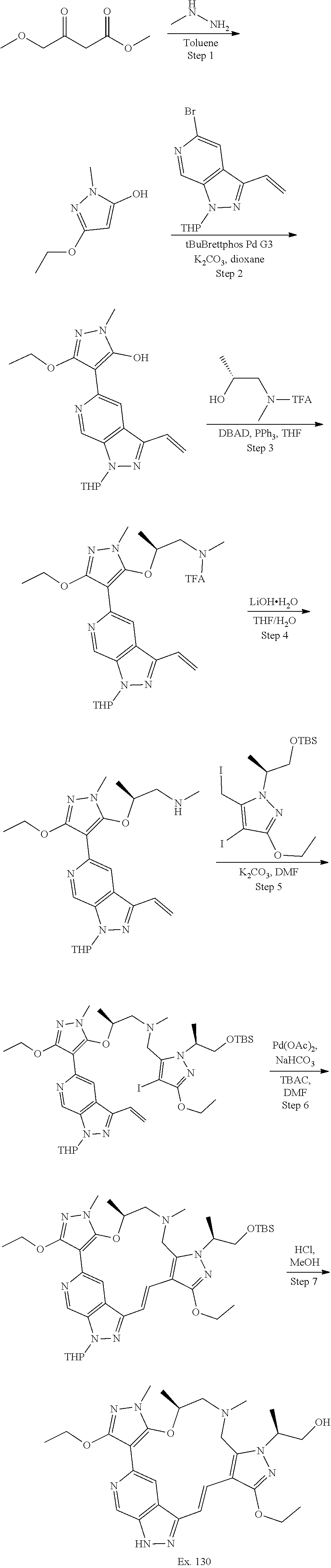

Step 1. To a solution of commercially available methyl 4-methoxy-3-oxo-butanoate (5.00 g, 34.2 mmol, 1 eq) in toluene (150 mL) was added dropwise methylhydrazine (3.94 g, 34.2 mmol, 4.5 mL, 40% purity, 1 eq) at 0° C. over 0.5 h. The resulting mixture was stirred at 100° C. for 1.5 h. On completion, the mixture was concentrated to give a residue. The residue was purified by column chromatography (SiO₂, PE:THF=1:0 to 1:1) to give 5-(methoxymethyl)-2-methyl-pyrazol-3-ol (3.90 g, 80% yield) as a yellow solid. ¹H NMR (400 MHz, CDCl₃) δ=4.18 (s, 2H), 3.40 (s, 3H), 3.32 (s, 3H), 3.29 (s, 2H).

Step 2. To a solution of 5-bromo-1-tetrahydropyran-2-yl-3-vinyl-pyrazolo[3,4-c]pyridine (1.81 g, 5.86 mmol, 1 eq), which was prepared according to the method described in Ex. 1, in dioxane (15 mL) was added 5-(methoxymethyl)-2-methyl-pyrazol-3-ol (1.00 g, 7.03 mmol, 1.2 eq), K₂CO₃ (2.43 g, 17.6 mmol, 3 eq) and [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (500 mg, 586 mol, 0.1 eq) under N₂. The mixture was stirred at 130° C. for 1 h under N₂ atmosphere. On completion, the mixture was filtered and concentrated to give a residue. The residue was purified by column chromatography (SiO₂, Dichloromethane:Methanol=1:0 to 10:1) to give 5-(methoxymethyl)-2-methyl-4-(1-tetrahydropyran-2-yl-3-vinyl-pyrazolo[3,4-c]pyridin-5-yl)pyrazol-3-ol (2.20 g, 85% yield) as a red oil. LCMS: (M+1:370.1).

Step 3. A mixture of 5-(methoxymethyl)-2-methyl-4-(1-tetrahydropyran-2-yl-3-vinyl-pyrazolo[3,4-c]pyridin-5-yl)pyrazol-3-ol (1.00 g, 2.71 mmol, 1 eq), commercially available 2,2,2-trifluoro-N-[(2R)-2-hydroxypropyl]-N-methyl-acetamide (1.00 g, 5.41 mmol, 2 eq), PPh₃ (1.42 g, 5.41 mmol, 2 eq) and DBAD (1.25 g, 5.41 mmol, 2 eq) was degassed and purged with N₂ for 3 times, then THE (10 mL) was added. The mixture was stirred at 25° C. for 1 h. On completion, the mixture was concentrated to give a residue. The residue was purified by column chromatography (SiO₂, Petroleum ether/Ethyl acetate=1:0 to 1:2) to give 2,2,2-trifluoro-N-[(2S)-2-[5-(methoxymethyl)-2-methyl-4-(1-tetrahydropyran-2-yl-3-vinyl-pyrazolo[3,4-c]pyridin-5-yl)pyrazol-3-yl]oxypropyl]-N-methyl-acetamide (1.00 g, 68% yield) as a yellow oil. LCMS: (M+1:537.3).

Step 4. To a solution of the product from Step 3 (950 mg, 1.77 mmol, 1 eq) in THF (9.5 mL) and H₂O (1.9 mL) was added LiOH·H₂O (223 mg, 5.31 mmol, 3 eq). The mixture was stirred at 25° C. for 1 h. On completion, the mixture was quenched with water (50 mL) and extracted with EA (50 mL×3). The combined organic layers were washed with brine (50 mL×2), dried over Na₂SO₄, filtered and concentrated to give a residue. The residue was purified by column chromatography (SiO₂, Dichloromethane:Methanol=1:0 to 5:1) to give (2S)-2-[5-(methoxymethyl)-2-methyl-4-(1-tetrahydropyran-2-yl-3-vinyl-pyrazolo[3,4-c]pyridin-5-yl)pyrazol-3-yl]oxy-N-methyl-propan-1-amine (580 mg, 74% yield) as a colorless oil. LCMS: (M+1:441.2).

Step 5. To a solution of the product from Step 4 (514 mg, 1.17 mmol, 1 eq) in DMF (5.3 mL) was added tert-butyl-[(2S)-2-[3-ethoxy-4-iodo-5-(iodomethyl)pyrazol-1-yl]propoxy]-dimethyl-silane (706 mg, 1.28 mmol, 1.1 eq), which was prepared according to the method described in Ex. 120, and K₂CO₃ (483 mg, 3.50 mmol, 3 eq). The mixture was stirred at 60° C. for 1 h. On completion, the mixture was quenched with water (20 mL) and extracted with EA (15 mL×3). The combined organic layers were washed with brine (15 mL×2), dried over Na₂SO₄, filtered and concentrated. The residue was purified by column chromatography (SiO₂, PE:THF=1:0 to 2:1) to give (2S)-N-[[2-[(1S)-2-[tert-butyl(dimethyl)silyl]oxy-methyl-ethyl]-5-ethoxy-4-iodo-pyrazol-3-yl]methyl]-2-[5-(methoxymethyl)-2-methyl-4-(1-tetrahydropyran-2-yl-3-vinyl-pyrazolo[3,4-c]pyridin-5-yl)pyrazol-3-yl]oxy-N-methyl-propan-1-amine (850 mg, 84% yield) as a yellow oil. LCMS: (M+1:863.4).

Step 6. A mixture of the product from Step 5 (200 mg, 232 μmol, 1 eq), NaHCO₃ (48.7 mg, 579 μmol, 2.5 eq), TBAC (64.4 mg, 232 μmol, 1 eq) and Pd(OAc)₂ (5.20 mg, 23.2 μmol, 0.1 eq) in DMF (10 mL) was degassed and purged with N₂ for 3 times, and then the mixture was stirred at 130° C. for 1 h under N₂ atmosphere. On completion, the mixture was quenched with water (40 mL) and extracted with EA (30 mL×3). The combined organic layers were washed with brine (30 mL×2), dried over Na₂SO₄. filtered and concentrated to give (10S,17E)-14-[(2S)-1-{[tert-butyl(dimethyl)silyl]oxy}propan-2-yl]-16-ethoxy-6-(methoxymethyl)-8,10,12-trimethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (250 mg, crude) as a red oil. LCMS: (M+1:735.5).

Step 7. To a solution of the product from Step 6 (100 mg, 0.136 mmol, 1 eq) in MeOH (1.5 mL) was added HCl (12 M, 0.5 mL, 44.1 eq). The mixture was stirred at 25° C. for 0.5 h. On completion, the mixture was concentrated to give a residue. The residue was purified by prep-HPLC (column: Waters xbridge 150*25 mm 10 um; mobile phase: [water(NH₄HCO₃)-ACN]; gradient:22%-52% B over 10 min) to give Ex. 130 (32.09 mg, 42.60% yield) as a white solid. ¹H NMR for Ex. 130 can be found in the below NMR table.

### Preparation of (2S)-2-[(10S,17E)-16-ethoxy-12-ethyl-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 145)

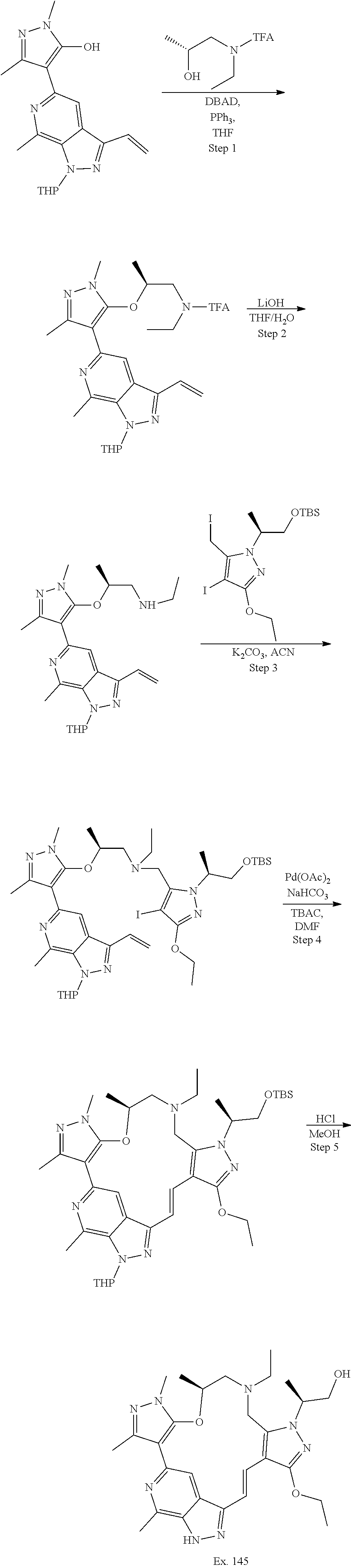

Step 1. To a solution of 2,5-dimethyl-4-(7-methyl-1-tetrahydropyran-2-yl-3-vinyl-pyrazolo[3,4-c]pyridin-5-yl)pyrazol-3-ol (500 mg, 1.41 mmol, 1 eq) from Ex. 47, N-ethyl-2,2,2-trifluoro-N-[(2R)-2-hydroxypropyl]acetamide (282 mg, 1.41 mmol, 1 eq) form Ex. 23, PPh₃ (557 mg, 2.12 mmol, 1.5 eq) and DBAD (653 mg, 2.83 mmol, 2 eq) was degassed and purged with N₂ for 3 times and added THE (5 mL). The mixture was stirred at 25° C. for 1 h. On completion, the mixture was concentrated to give a residue. The residue was purified by column chromatography (SiO₂, PE/THF=1:0 to 1:1) to give N-((2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)propyl)-N-ethyl-2,2,2-trifluoroacetamide (1.27 g, 0.781 mmol, 55% yield, 33% purity) as a yellow oil. LCMS: (M+1: 535.1).

Step 2. To a solution of N-((2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)propyl)-N-ethyl-2,2,2-trifluoroacetamide (1.10 g, 2.06 mmol, 1 eq) in THE (5 mL) and H₂O (1 mL) was added LiOH·H₂O (432 mg, 10.3 mmol, 5 eq). The mixture was stirred at 25° C. for 16 h. On completion, the mixture was quenched with H₂O (3 mL) and extracted with 2-Me THE (5 mL×3). The combined organic phase was dried over anhydrous sodium sulfate, filtered, and concentrated to give a residue. The residue was purified by column chromatography (SiO₂, DCM:MeOH=1:0 to 10:1) to give (2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-ethylpropan-1-amine (355 mg, 0.809 mmol, 39% yield) as a green oil. LCMS: (M+1: 439.4).

Step 3. To a solution of (2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-ethylpropan-1-amine (335 mg, 0.764 mmol, 1 eq) and tert-butyl-[(2S)-2-[3-ethoxy-4-iodo-5-(iodomethyl)pyrazol-1-yl]propoxy]-dimethyl-silane (504 mg, 0.917 mmol, 1.2 eq) from Ex. 120 in ACN (5 mL) was added K₂CO₃ (317 mg, 2.29 mmol, 3 eq). The mixture was stirred at 80° C. for 1 h. On completion, the mixture was concentrated to give a residue. The residue was purified by column chromatography (SiO₂, PE/THF=1:0 to 3:1) to give (2S)-N-((1-((S)-1-((tert-butyldimethylsilyl)oxy)propan-2-yl)-3-ethoxy-4-iodo-1H-pyrazol-5-yl)methyl)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-ethylpropan-1-amine (310 mg, 0.36 mmol, 47% yield) as a green oil. LCMS (M+1: 861.4).

Step 4. To a mixture of (2S)-N-((1-((S)-1-((tert-butyldimethylsilyl)oxy)propan-2-yl)-3-ethoxy-4-iodo-1H-pyrazol-5-yl)methyl)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-ethylpropan-1-amine (290 mg, 0.337 mmol, 1 eq) in DMF (3 mL) was added TBAC (93.6 mg, 0.337 mmol, 1 eq), NaHCO₃ (70.8 mg, 0.842 mmol, 2.5 eq) and Pd(OAc)₂ (15.1 mg, 0.067 mmol, 0.2 eq). The mixture was degassed and purged with N₂ for 3 times, and then the mixture was stirred at 130° C. for 1 h under N₂ atmosphere. On completion, the mixture was quenched with water (5 mL) and extracted with 2-Me THE (5 mL×3). The combined organic phase was dried over anhydrous sodium sulfate, filtered, and concentrated to give a residue. The residue was purified by column chromatography (SiO₂, PE/EA=1:0 to 1:2) to give (10S,17E)-14-[(2S)-1-{[tert-butyl(dimethyl)silyl]oxy}propan-2-yl]-16-ethoxy-12-ethyl-6,8,10,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (130 mg, 0.177 mmol, 53% yield) as a yellow oil. LCMS: (M+1: 733.4).

Step 5. To a solution of (10S,17E)-14-[(2S)-1-{[tert-butyl(dimethyl)silyl]oxy}propan-2-yl]-16-ethoxy-12-ethyl-6,8,10,20-tetramethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (110 mg, 0.15 mmol, 1 eq) in MeOH (1.5 mL) was added HCl (0.5 mL). The mixture was stirred at 25° C. for 10 mins. On completion, the mixture was purified by prep-HPLC (column: CD07-Daisogel SP-100-8-ODS-PK 150*25*10 um; mobile phase: [water(NH₄HCO₃)-ACN]; gradient:36%-66% B over 10 min) to give (2S)-2-[(10S,17E)-16-ethoxy-12-ethyl-6,8,10,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (38.79 mg, 0.073 mmol, 48% yield) as a white solid. ¹H NMR for Ex. 145 can be found in the below NMR table.

### Preparation of (17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecine (Ex. 146)

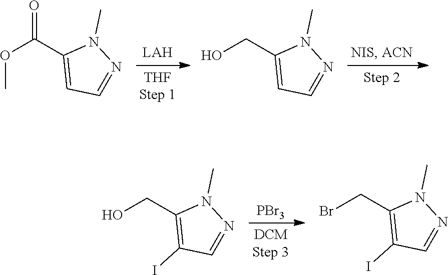

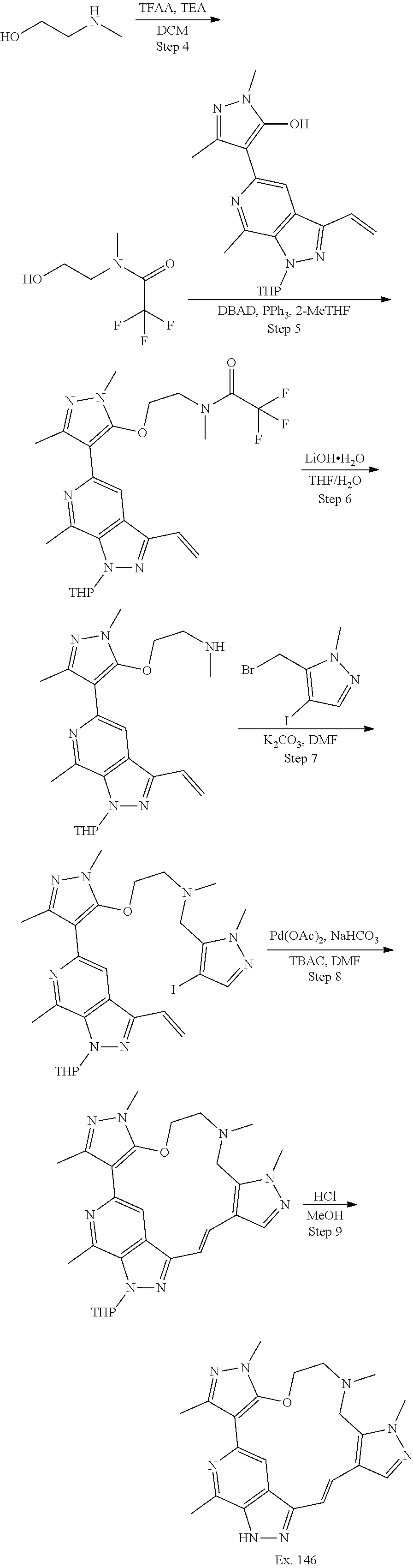

Step 1. To a solution of methyl 1-methyl-1H-pyrazole-5-carboxylate (10.0 g, 71.4 mmol, 1 eq) in THE (100 mL) was added LAH (2.5 M in THF, 42.8 mL, 1.5 eq) at 0° C. The mixture was stirred at 0° C. for 6 hr. On completion, the reaction mixture was quenched with H₂O (4 mL) dropwise at 0° C. and adjusted with 15% NaOH (4 mL), then added H₂O (4 mL) and Na₂SO₄, stirred at 25° C. for 20 min and filtered. The filtrate was concentrated under reduced pressure to give (1-methyl-1H-pyrazol-5-yl)methanol (7.88 g, crude) as a colorless oil. ¹H NMR (400 MHz, DMSO-d₆) δ=7.31 (s, 1H), 6.17 (s, 1H), 5.31 (t, J=5.2 Hz, 1H), 4.52 (d, J=5.2 Hz, 2H), 3.79 (s, 3H).

Step 2. To a solution of (1-methyl-1H-pyrazol-5-yl)methanol (7.30 g, 65.1 mmol, 1 eq) in ACN (50 mL) was added NIS (17.6 g, 78.1 mmol, 1.2 eq) at 0° C. The mixture was stirred at 25° C. for 16 hr. On completion, the mixture was quenched with saturated solution of Na₂SO₃ (30 mL), partitioned between water (300 mL) and ethyl acetate(100 mL×7). The combined organic phase was washed with brine (100 mL), dried over anhydrous sodium sulfate, filtered and concentrated to give (4-iodo-1-methyl-1H-pyrazol-5-yl)methanol (13.0 g, 54.6 mmol, 84% yield) as a white solid. 1H NMR (400 MHz, DMSO-d₆) δ=7.43 (s, 1H), 5.32 (s, 1H), 4.48 (s, 2H), 3.87 (s, 3H).

Step 3. To a solution of (4-iodo-1-methyl-1H-pyrazol-5-yl)methanol (5.00 g, 21.0 mmol, 1 eq) in DCM (50 mL) was added tribromophosphane (6.82 g, 25.2 mmol, 1.2 eq) at 0° C. The mixture was stirred at 0° C. for 6 h. On completion, the mixture was quenched with sat. NaHCO₃ (100 mL), extracted with DCM (100 mL). The organic layer was washed with water (100 mL), brine (2×200 mL). The organic layer was dried over sodium sulfate, concentrated in vacuum to afford 5-(bromomethyl)-4-iodo-1-methyl-1H-pyrazole (4.50 g, 15.0 mmol, 71% yield) as a white solid. LCMS: (M+1=300.8)

Step 4. To a solution of 2-(methylamino)ethan-1-ol (15.0 g, 200 mmol, 1 eq) in DCM (100 mL) was added TEA (101 g, 999 mmol, 5 eq) and (2,2,2-trifluoroacetyl) 2,2,2-trifluoroacetate (83.9 g, 399 mmol, 2 eq) at 0° C. The mixture was stirred at 25° C. for 16 hr. On completion, the reaction mixture was partitioned between mixed solution of dichloromethane:methanol=10:1 (100 mL×3) and water (200 mL). The combined organic phase was dried over anhydrous sodium sulfate, filtered and concentrated to give a residue. The residue was purified by column chromatography (SiO₂, Petroleum ether/THF=1:0 to 0:1) to give 2,2,2-trifluoro-N-(2-hydroxyethyl)-N-methylacetamide (4.60 g, 26.9 mmol, 13% yield) as a yellow oil. ¹H NMR (400 MHz, CDCl3) δ=4.21 (s, 1H), 3.80-3.70 (m, 2H), 3.58-3.46 (m, 2H), 3.20-2.99 (m, 3H); LCMS: (M+1=172.1).

Step 5. A mixture of 2,2,2-trifluoro-N-(2-hydroxyethyl)-N-methylacetamide (3.90 g, 22.8 mmol, 1.8 eq), 1,3-dimethyl-4-(7-methyl-1-(tetrahydro-2H-pyran-2-yl)-3-vinyl-1H-pyrazolo[3,4-c]pyridin-5-yl)-1H-pyrazol-5-ol, which was prepared according to the method described in Ex. 47 (4.47 g, 12.7 mmol, 1 eq), and PPh₃ (7.31 g, 27.9 mmol, 2.2 eq) in 2-MeTHF (100 mL) was degassed and purged with N₂ 3 times, and then DBAD (6.41 g, 27.9 mmol, 2.2 eq) was added into the mixture at 0° C. The mixture stirred at 25° C. for 2 h under N₂ atmosphere. On completion, the mixture was concentrated in vacuo to give a residue. The residue was purified by column chromatography (SiO₂, Petroleum ether/THF=1:0 to 0:1) to give N-(2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)ethyl)-2,2,2-trifluoro-N-methylacetamide (8.00 g, crude) as a red solid. LCMS: (M+1=507.3).

Step 6. To a solution of N-(2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)ethyl)-2,2,2-trifluoro-N-methylacetamide (8.00 g, 9.48 mmol, 1 eq) in THF (80 mL) was added LiOH·H₂O (1.99 g, 47.4 mmol, 5 eq) in H₂O (16 mL). The mixture was stirred at 25° C. for 30 min. On completion, the reaction mixture was separated, the water phase was partitioned between ethyl acetate (20 mL×3) and water (20 mL). The combined organic phase was dried over anhydrous sodium sulfate, filtered, and concentrated to give a residue. The residue was purified by column chromatography (SiO₂, DCM/MeOH=1:0 to 2:1) to give 2-((1,3-dimethyl-4-(7-methyl-1-(tetrahydro-2H-pyran-2-yl)-3-vinyl-H-pyrazolo[3,4-c]pyridin-5-yl)-1H-pyrazol-5-yl)oxy)-N-methylethan-1-amine (3.50 g, 8.01 mmol, 85% yield, 94% purity) as an orange oil. ¹H NMR (400 MHz, DMSO-d₆) δ=7.65 (s, 1H), 7.30-7.16 (m, 1H), 6.10-5.92 (m, 2H), 5.71-5.59 (m, 1H), 3.93 (d, J=11.2 Hz, 4H), 3.64 (s, 3H), 2.91-2.84 (m, 2H), 2.76 (s, 3H), 2.37-2.25 (m, 6H), 2.13-1.97 (m, 2H), 1.83-1.70 (m, 1H), 1.69-1.52 (m, 2H); LCMS: (M+1=411.2).

Step 7. To a solution of 2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylethan-1-amine (400 mg, 0.974 mmol, 1 eq) and 5-(bromomethyl)-4-iodo-1-methyl-1H-pyrazole, which was prepared in Step 3 (293 mg, 0.974 mmol, 1 eq) in DMF (4 mL) was added K₂CO₃(135 mg, 0.974 mmol, 1 eq). The mixture was stirred at 80° C. for 1 hr. On completion, the reaction mixture was partitioned between ethyl acetate/dichloromethane (20 mL×3) and water (40 mL). The combined organic phase was washed with brine (40 mL), dried over anhydrous sodium sulfate, filtered, and concentrated to give a residue. The residue was purified by column chromatography (SiO₂, Petroleum ether/THF=1:0 to 0:1) to give 2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-((4-iodo-1-methyl-1H-pyrazol-5-yl)methyl)-N-methylethan-1-amine (480 mg, 0.761 mmol, 78% yield) as a colorless oil. LCMS: (M+1=631.3)

Step 8. A mixture of 2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-((4-iodo-1-methyl-1H-pyrazol-5-yl)methyl)-N-methylethan-1-amine (420 mg, 0.666 mmol, 1 eq), TBAC (185.13 mg, 0.666 mmol, 1 eq), NaHCO₃ (140 mg, 1.67 mmol, 2.5 eq) and Pd(OAc)₂ (29.9 mg, 0.133 mmol, 0.2 eq) in DMF (5 mL) was degassed and purged with N₂ 3 times, and then the mixture was stirred at 130° C. for 30 min under N₂ atmosphere. On completion, the reaction mixture was partitioned between ethyl acetate (30 mL×3) and water (50 mL). The combined organic phase was washed with brine (50 mL×2), dried over anhydrous sodium sulfate, filtered and concentrated to give (17E)-6,8,12,14,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (600 mg, crude) as a brown oil without further purification. LCMS: (M+1=503.3).

Step 9. To a solution (17E)-6,8,12,14,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecine (590 mg, 0.798 mmol, 1 eq) in MeOH (4.2 mL) was added HCl (aq. 12 M, 3.23 mL, 48.57 eq). The mixture was stirred at 25° C. for 30 min. On completion, the mixture was concentrated in vacuo to give a residue. The residue was purified by prep-HPLC purification (column: Phenomenex luna C18 150*25 mm*10 um; mobile phase: [water(FA)-ACN]; gradient:5%-35% B over 8 min) to give (17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecine (52.79 mg, 0.101 mmol, 12.70% yield, 89.21% purity, FA) as a yellow solid (Ex. 146). The NMR data for Ex. 146 is shown in the table below.

### Preparation of {[(17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 160)

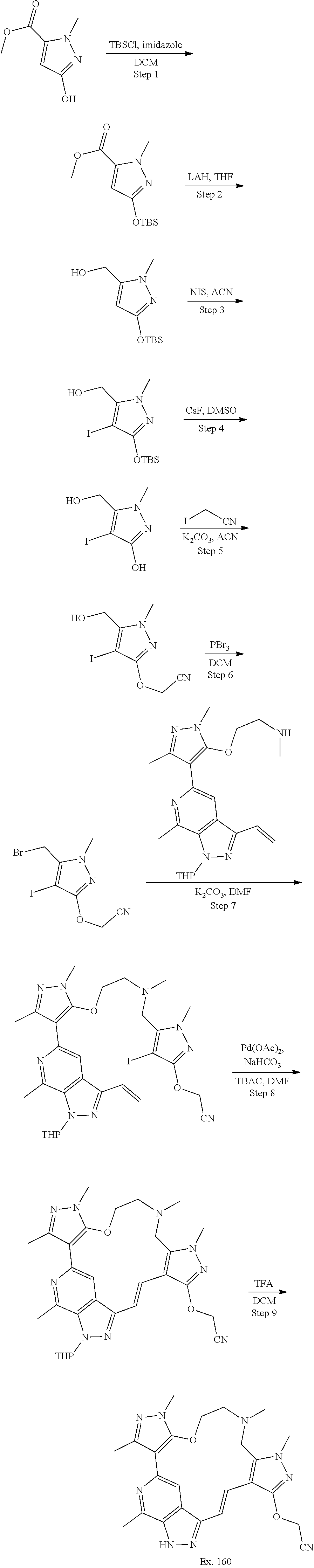

Step 1. To a solution of methyl 3-hydroxy-1-methyl-1H-pyrazole-5-carboxylate (5.00 g, 32.0 mmol, 1 eq) in DCM (50 mL) was added TBSCl (7.24 g, 48.0 mmol, 5.91 mL, 1.5 eq) and imidazole (6.54 g, 96.1 mmol, 3 eq). The mixture was stirred at 25° C. for 2 h. On completion, the reaction mixture was filtered and concentrated under reduced pressure to give a residue. The residue was purified by flash silica gel chromatography (0-5% Ethyl acetate/Petroleum ether) to give methyl 3-((tert-butyldimethylsilyl)oxy)-1-methyl-1H-pyrazole-5-carboxylate (6.40 g, 23.7 mmol, 74% yield, 100% purity) as a colorless oil. ¹H NMR (400 MHz, CDCl3-d) 6=6.13 (s, 1H), 4.02 (s, 3H), 3.86 (s, 3H), 0.98 (s, 9H), 0.25 (s, 6H); LCMS: (M+1:271.0).

Step 2. To a solution of methyl 3-((tert-butyldimethylsilyl)oxy)-1-methyl-1H-pyrazole-5-carboxylate (6.00 g, 22.2 mmol, 1 eq) in THF (60 mL) was added LAH (2.5 M in THF, 9.76 mL, 1.1 eq) at 0° C. The mixture was stirred at 0° C. for 1 h. On completion, the reaction mixture was quenched by addition of 15% NaOH (5 mL). The combined organic layers were dried over Na₂SO₄, filtered, and concentrated under reduced pressure to give a residue. The crude product (3-((tert-butyldimethylsilyl)oxy)-1-methyl-1H-pyrazol-5-yl)methanol (4.68 g, 19.3 mmol, 87% yield) was obtained as a light yellow oil and it was used into the next step without further purification. LCMS: (M+1:243.2).

Step 3. To a solution of (3-((tert-butyldimethylsilyl)oxy)-1-methyl-1H-pyrazol-5-yl)methanol (4.58 g, 18.9 mmol, 1 eq) in ACN (45 mL) was added NIS (4.68 g, 20.8 mmol, 1.1 eq) at 0° C. The mixture was stirred at 25° C. for 1 h. On completion, the mixture was poured into Na₂SO₃ (80 mL) aqueous solution, the aqueous phase was extracted with ethyl acetate (60 mL×2). The combined organic phase was washed with brine (40 mL×2), dried over anhydrous Na₂SO₄, filtered, and concentrated under vacuum. The crude product (3-((tert-butyldimethylsilyl)oxy)-4-iodo-1-methyl-1H-pyrazol-5-yl)methanol (5.8 g, crude) was obtained as a yellow liquid and it was used into the next step without further purification. LCMS: (M+1:369.2).

Step 4. To a solution of (3-((tert-butyldimethylsilyl)oxy)-4-iodo-1-methyl-1H-pyrazol-5-yl)methanol (5.80 g, 11.0 mmol, 1 eq) in DMSO (60 mL) was added CsF (8.37 g, 55.1 mmol, 5 eq). The mixture was stirred at 25° C. for 1 h. On completion, the reaction mixture was adjusted to pH-5 with citric acid. The mixture was extracted with Ethyl acetate (50 mL×5) and the combined organic phase was washed with brine (100 mL×2), dried over anhydrous sodium sulfate, filtered, and concentrated in vacuum. The residue was purified by flash silica gel chromatography (0-56% THF/Petroleum ether) to give 5-(hydroxymethyl)-4-iodo-1-methyl-1H-pyrazol-3-ol (1.6 g, 6.30 mmol, 57% yield) as a yellow solid. LCMS: (M+1:254.8).

Step 5. To a solution of 5-(hydroxymethyl)-4-iodo-1-methyl-1H-pyrazol-3-ol (1.40 g, 5.51 mmol, 1 eq) and 2-iodoacetonitrile (1.38 g, 8.27 mmol, 1.5 eq) in ACN (15 mL) was added K₂CO₃ (2.29 g, 16.5 mmol, 3 eq). The mixture was stirred at 80° C. for 1 h. On completion, the mixture was concentrated under vacuum. The residue was purified by flash silica gel chromatography (0-45% THE/Petroleum ether) to give 2-((5-(hydroxymethyl)-4-iodo-1-methyl-1H-pyrazol-3-yl)oxy)acetonitrile (1.13 g, 3.66 mmol, 66% yield, 95% purity) as a yellow solid. ¹H NMR (400 MHz, CDCl₃-d) 6=4.91 (s, 2H), 4.63 (s, 2H), 3.93-3.85 (m, 3H); LCMS: (M+1:293.9).

Step 6. To a solution of 2-((5-(hydroxymethyl)-4-iodo-1-methyl-1H-pyrazol-3-yl)oxy)acetonitrile (800 mg, 2.73 mmol, 1 eq) in DCM (8 mL) was added PBr₃ (1.11 g, 4.09 mmol, 1.5 eq). The mixture was stirred at 0° C. for 1 h. On completion, the mixture was quenched with Sat. NaHCO₃ (30 mL), extracted with DCM (30 mL). The organic layer was washed with water (30 mL), brine (2×20 mL). The organic layer was dried over sodium sulfate, concentrated in vacuum to afford crude. The residue was purified by flash silica gel chromatography (0-25% THE/Petroleum ether) to give 2-((5-(bromomethyl)-4-iodo-1-methyl-1H-pyrazol-3-yl)oxy)acetonitrile (245 mg, 0.344 mmol, 13% yield, 50% purity) as a yellow solid. LCMS: (M+1:355.7).

Step 7. To a solution of 2-((5-(bromomethyl)-4-iodo-1-methyl-1H-pyrazol-3-yl)oxy)acetonitrile (91.1 mg, 0.256 mmol, 1.5 eq) and 2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylethan-1-amine, which was prepared following the method described in Ex. 146 (70.0 mg, 0.171 mmol, 1 eq) in DMF (1 mL) was added K₂CO₃ (70.7 mg, 0.512 mmol, 3 eq). The mixture was stirred at 80° C. for 1 h. On completion, the mixture was poured into water (5 mL) aqueous solution, the aqueous phase was extracted with ethyl acetate (5 mL×2). The combined organic phase was washed with brine (5 mL×2), dried over anhydrous Na₂SO₄, filtered, and concentrated under vacuum. The residue was purified by flash silica gel chromatography (0-30% THE/Petroleum ether) to give 2-((5-(((2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)ethyl)(methyl)amino)methyl)-4-iodo-1-methyl-1H-pyrazol-3-yl)oxy)acetonitrile (50 mg, 0.073 mmol, 36% yield, 85% purity) as a yellow solid. LCMS: (M+1:686.2).

Step 8. A mixture of 2-((5-(((2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)ethyl)(methyl)amino)methyl)-4-iodo-1-methyl-1H-pyrazol-3-yl)oxy)acetonitrile (45.0 mg, 0.066 mmol, 1 eq), TBAC (18.2 mg, 0.066 mmol, 1 eq), NaHCO₃ (13.8 mg, 0.164 mmol, 2.5 eq) and Pd(OAc)₂ (1.47 mg, 0.0066 mmol, 0.1 eq) in DMF (0.5 mL) was degassed and purged with N₂ 3 times, and then the mixture was stirred at 130° C. for 1 h under N₂ atmosphere. On completion, the mixture was poured into water (5 mL) aqueous solution, the aqueous phase was extracted with ethyl acetate (5 mL×2). The combined organic phase was washed with brine (5 mL×2), dried over anhydrous Na₂SO₄, filtered, and concentrated under vacuum. Compound {[(17E)-6,8,12,14,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (40 mg, crude) was obtained as a black brown oil. The crude product was used into the next step without further purification. LCMS: (M+1:558.3).

Step 9. To a solution of {[(17E)-6,8,12,14,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile),2(6),3,12(16),14,17,19,22(26),23-nonaen-15-yl]oxy]acetonitrile (40.0 mg, 0.072 mmol, 1 eq) in DCM (0.6 mL) was added TFA (307 mg, 2.69 mmol, 0.2 mL, 37.5 eq). The mixture was stirred at 25° C. for 0.5 h. On completion, the mixture was poured into NaHCO₃ (3 mL) aqueous solution, the aqueous phase was extracted with ethyl acetate (3 mL×2). The combined organic phase was washed with brine (3 mL×2), dried over anhydrous Na₂SO₄, filtered, and concentrated under vacuum. The residue was purified by prep-HPLC to give {[(17E)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (1.25 mg, 0.0021 mmol, 2.91% yield, 93.01% purity) as a yellow solid (Ex. 160). The NMR data for Ex. 160 is shown in the table below.

### Preparation of (2S)-1-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n∥1,4]oxazacyclopentadecin-14-yl]propan-2-ol (Ex. 181)

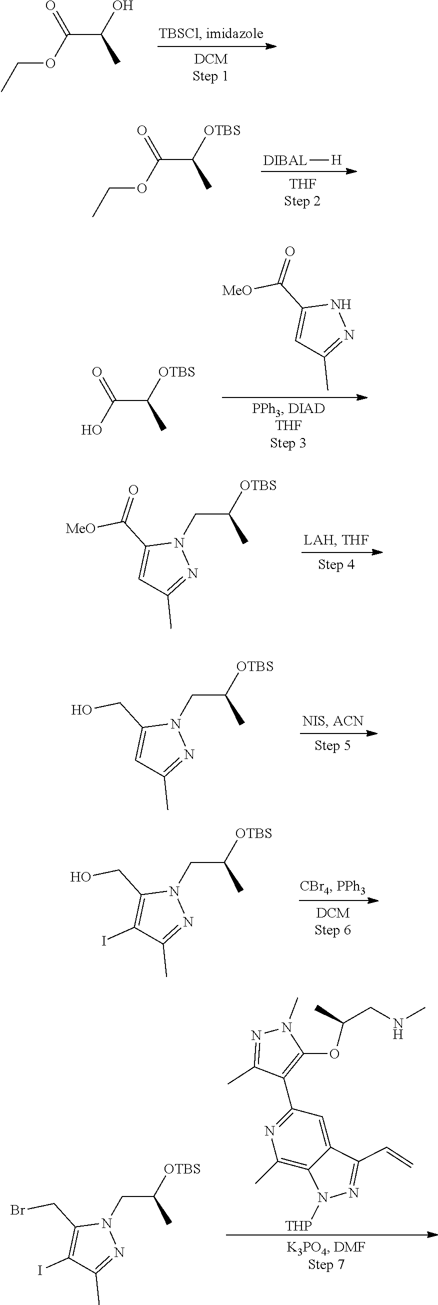

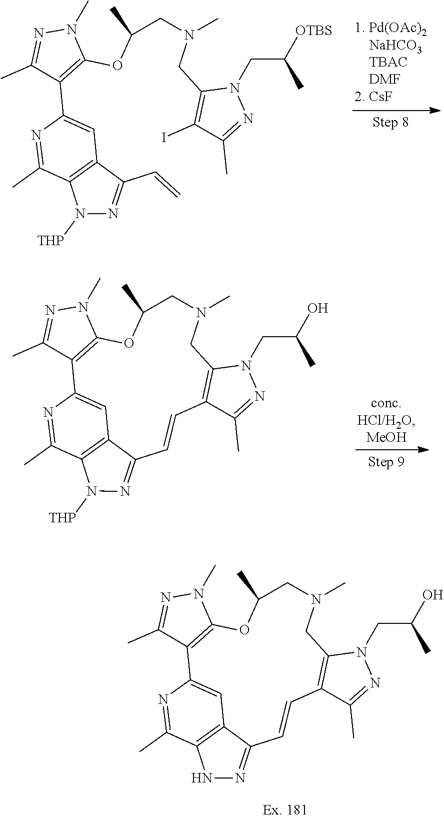

Step 1. To a solution of ethyl (2S)-2-hydroxypropanoate (5.00 g, 42.3 mmol, 1 eq) in DCM (50 mL) was added imidazole (8.64 g, 127 mmol, 3 eq), and then TBSCl (9.57 g, 63.5 mmol, 1.5 eq) was added at 0° C. The mixture was stirred at 25° C. for 2 h. On completion, the reaction mixture was partitioned between DCM (60 mL×3) and water (50 mL). The combined organic phase was dried over anhydrous sodium sulfate, filtered, and concentrated to give a residue. The residue was purified by column chromatography (SiO₂, PE/EA=10:1 to 10:1) to give ethyl (S)-2-((tert-butyldimethylsilyl)oxy)propanoate (9.70 g, 41.7 mmol, 99% yield) as a brown solid. ¹H NMR (400 MHz, DMSO-d₆) δ=4.38-4.29 (m, 1H), 4.14-4.04 (m, 2H), 1.28 (d, J=6.8 Hz, 3H), 1.19 (t, J=7.2 Hz, 3H), 0.87-0.85 (m, 9H), 0.05 (d, J=3.2 Hz, 6H).

Step 2. To a solution of ethyl (S)-2-((tert-butyldimethylsilyl)oxy)propanoate (8.70 g, 37.4 mmol, 1 eq) in THE (87 mL) was added DIBAL-H (1 M, 74.8 mL, 2 eq) at 0° C. under N₂ atmosphere. The mixture was stirred 25° C. for 4 h. On completion, water (3 mL), 15% NaOH (3 mL) and water (7.5 mL) were added successively, and the mixture was filtered and concentrated to give a residue. The residue was purified by column chromatography (SiO₂, Petroleum ether/Ethyl acetate=10:1 to 10:1) to give (S)-2-((tert-butyldimethylsilyl)oxy)propan-1-ol (4.87 g, 25.6 mmol, 68% yield) as a white oil. ¹H NMR (400 MHz, DMSO-d₆) δ=4.53 (t, J=5.6 Hz, 1H), 3.80-3.66 (m, 1H), 3.30-3.09 (m, 2H), 1.09-0.99 (m, 3H), 0.87-0.84 (m, 8H), 0.04 (d, J=0.8 Hz, 6H).

Step 3. A mixture of (S)-2-((tert-butyldimethylsilyl)oxy)propan-1-ol (12.2 g, 64.2 mmol, 1.5 eq), methyl 3-methyl-1H-pyrazole-5-carboxylate (6.00 g, 42.8 mmol, 1 eq), and PPh₃ (33.6 g, 128 mmol, 3 eq) in THE (50 mL) was degassed and purged with N₂ 3 times. To the mixture was added DIAD (85.6 mmol, 16.6 mL, 2 eq) at 0° C., and then the mixture was stirred at 25° C. for 2 h under N₂ atmosphere. On completion, the reaction mixture was concentrated in vacuo to give a residue. The residue was purified by column chromatography (SiO₂, Petroleum ether/Ethyl acetate=1/0 to 90/10) to give methyl (S)-1-(2-((tert-butyldimethylsilyl)oxy)propyl)-3-methyl-1H-pyrazole-5-carboxylate (12.0 g, 37.6 mmol, 87% yield, 98% purity) as colorless oil. LCMS: (M+1: 313.1).

Step 4. A mixture of (S)-1-(2-((tert-butyldimethylsilyl)oxy)propyl)-3-methyl-1H-pyrazole-5-carboxylate (10.0 g, 32.0 mmol, 1 eq) in THE (100 mL) was degassed and purged with N₂ 3 times, LiAlH₄ (2.5 M, 19.2 mL, 1.5 eq) was added to the mixture at 0° C., and then the mixture was stirred at 25° C. for 2 h under N₂ atmosphere. On completion, to the mixture was added with H₂O (0.2 mL), NaOH (15% in water, 0.2 mL) and H₂O (0.6 mL) successively. The mixture was dried over anhydrous Na₂SO₄, filtered and concentrated in vacuo to give (S)-(1-(2-((tert-butyldimethylsilyl)oxy)propyl)-3-methyl-1H-pyrazol-5-yl)methanol (9.00 g, 31.6 mmol, 98% yield) as colorless oil. LCMS: (M+1: 285.0).

Step 5. To a solution of (S)-(1-(2-((tert-butyldimethylsilyl)oxy)propyl)-3-methyl-1H-pyrazol-5-yl)methanol (8.80 g, 30.9 mmol, 1 eq) in ACN (90 mL) was added NIS (7.66 g, 34.0 mmol, 1.1 eq). The mixture was stirred at 25° C. for 2 h. On completion, the reaction mixture was quenched by addition sat. Na₂SO₃ (50 mL) at 0° C., and then diluted with H₂O (50 mL) and extracted with Ethyl acetate (3×50 mL). The combined organic layers were washed with brine (3×50 mL), dried over anhydrous Na₂SO₄, filtered, and concentrated in vacuo to give a residue. The residue was purified by column chromatography (SiO₂, Petroleum ether/Ethyl acetate=100/1 to 90/10) to give (S)-(1-(2-((tert-butyldimethylsilyl)oxy)propyl)-4-iodo-3-methyl-1H-pyrazol-5-yl)methanol (10.1 g, 24.3 mmol, 78% yield, 99% purity) as a white solid. ¹H NMR (400 MHz, DMSO-d₆) δ=5.27 (t, J=4.8 Hz, 1H), 4.59-4.47 (m, 1H), 4.43-4.32 (m, 1H), 4.21-3.98 (m, 3H), 2.09 (s, 3H), 1.11 (d, J=5.6 Hz, 3H), 0.74 (s, 9H), −0.11 (s, 3H), −0.32 (s, 3H); LCMS: (M+1: 411.2).

Step 6. To a solution of (S)-(1-(2-((tert-butyldimethylsilyl)oxy)propyl)-4-iodo-3-methyl-1H-pyrazol-5-yl)methanol (3.00 g, 7.31 mmol, 1 eq) and CBr₄ (2.91 g, 8.77 mmol, 1.2 eq) in DCM (30 mL) was degassed and purged with N₂ for 3 times, then PPh₃ (3.83 g, 14.6 mmol, 2 eq) was added to the mixture. The mixture was stirred at 25° C. for 2 h under N₂ atmosphere. On completion, the reaction mixture was concentrated in vacuo to give a residue. And then the residue was purified by column chromatography (SiO₂, Petroleum ether/Ethyl acetate=100/1 to 90/10) to give (S)-5-(bromomethyl)-1-(2-((tert-butyldimethylsilyl)oxy)propyl)-4-iodo-3-methyl-1H-pyrazole (2.60 g, 5.44 mmol, 74% yield, 99% purity) as a white solid. ¹H NMR (400 MHz, DMSO-d₆) δ=4.75-4.55 (m, 2H), 4.21-3.96 (m, 3H), 2.10 (s, 3H), 1.14 (d, J=6.0 Hz, 3H), 0.75 (s, 9H), −0.09 (s, 3H), −0.29 (s, 3H); LCMS: (M+1: 474.6).

Step 7. To a solution of (S)-5-(bromomethyl)-1-(2-((tert-butyldimethylsilyl)oxy)propyl)-4-iodo-3-methyl-1H-pyrazole (1.00 g, 2.36 mmol, 1 eq) and (2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylpropan-1-amine, which was prepared following the method described in Ex. 125 (1.34 g, 2.83 mmol, 1.2 eq) in DMF (10 mL) was added K₃PO₄ (1.00 g, 4.71 mmol, 2 eq). The mixture was stirred at 60° C. for 0.5 h. On completion, the mixture was filtered, the filtrate was diluted with water (30 mL) and extracted with EA (30 mL×3). The combined organic phase was dried over anhydrous sodium sulfate, filtered, and concentrated to give a residue. The residue was purified by column chromatography (SiO₂, (PE:THF):MeOH=(2: 1): 0.01 to (2: 1): 0.01) to give (2S)-N-((1-((S)-2-((tert-butyldimethylsilyl)oxy)propyl)-4-iodo-3-methyl-1H-pyrazol-5-yl)methyl)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylpropan-1-amine (1.60 g, 1.96 mmol, 83% yield) as a colorless oil. LCMS: (M+1:817.6).

Step 8. To a mixture of (2S)-N-((1-((S)-2-((tert-butyldimethylsilyl)oxy)propyl)-4-iodo-3-methyl-1H-pyrazol-5-yl)methyl)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methylpropan-1-amine (600 mg, 0.734 mmol, 1 eq) in DMF (6 mL) was added NaHCO₃ (154 mg, 1.84 mmol, 2.5 eq), TBAC (204 mg, 70.34 mmol, 1 eq) and Pd(OAc)₂ (32.9 mg, 0.147 mmol, 0.2 eq), the mixture was stirred at 130° C. for 0.5 h under N₂ atmosphere. On completion of the Heck reaction (monitored by LCMS), the reaction mixture was cooled to 25° C. and CsF (558 mg, 3.67 mmol, 5 eq) was added, and the mixture was stirred at 60° C. for 12 h. On completion, the mixture was filtered and diluted with water (18 mL), and extracted with EA (18 mL×3). The combined organic phase was dried over anhydrous sodium sulfate, filtered, and concentrated to give a residue. The residue was purified by column chromatography (SiO₂, DCM:MeOH=15:1) to give (2S)-1-[(10S,17E)-6,8,10,12,16,20-hexamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol (400 mg, crude) as a yellow solid. LCMS: (M+1: 575.3).

Step 9. To a solution of (2S)-1-[(10S,17E)-6,8,10,12,16,20-hexamethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol (270 mg, 0.470 mmol, 1 eq) in MeOH (3 mL) was added cone. HCl (12 M, 0.6 mL, 15.3 eq). The mixture was stirred at 0° C. for 1 h. On completion, the reaction mixture was diluted with DCM (30 mL), and the mixture was quenched by sat. NaHCO₃ to adjust pH=7 at 0° C., followed by extraction with (MeOH/DCM=1/15) (6 mL×3). The combined organic layers were washed with brine (10 mL×2), dried over Na₂SO₄, filtered, and concentrated under reduced pressure to give a residue. The residue was purified by reversed phase column to give (2S)-1-[(10S,17E)-6,8,10,12,16,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-2-ol (124.53 mg, 0.252.13 mmol, 53.7% yield, 99.3% purity) as a yellow solid (Ex. 181); LCMS: (M+1:491.2). ¹H NMR for Ex. 181 can be found in the below NMR table.

General Method D: using Ex. 2 as example.

### Preparation of 2-[(11S,17E)-16-ethoxy-6,8,11-trimethyl-12-(propan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 2)

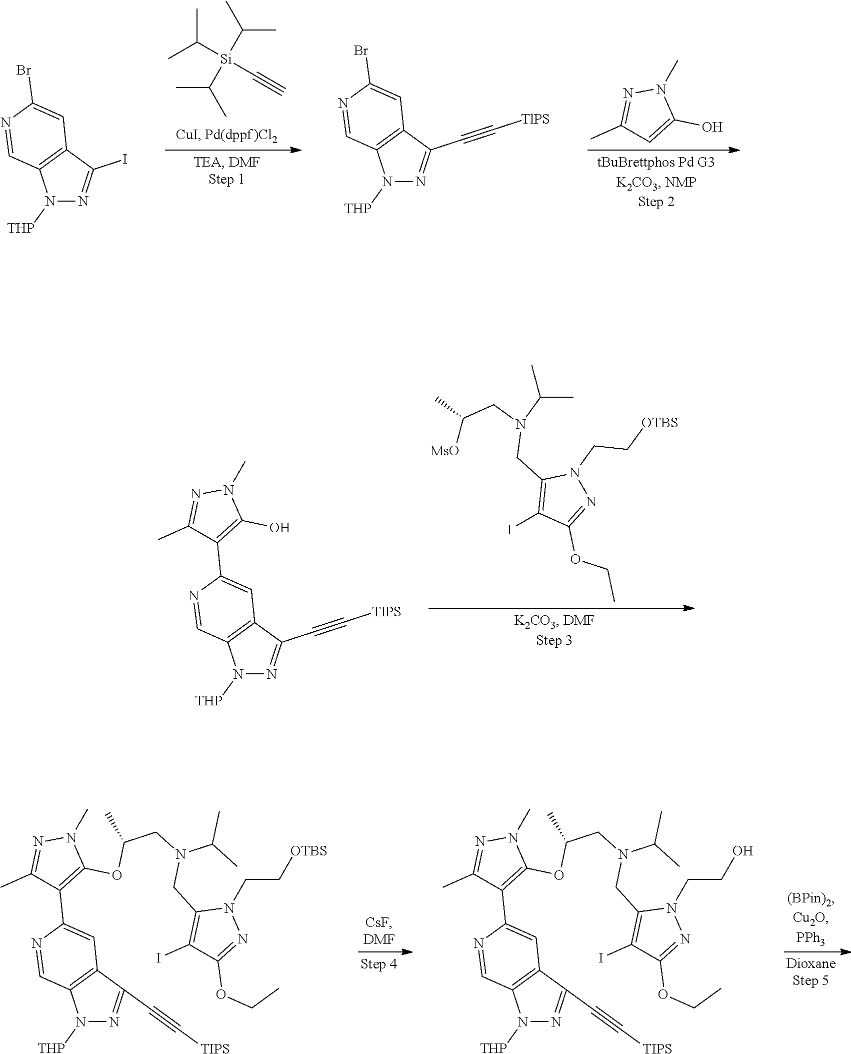

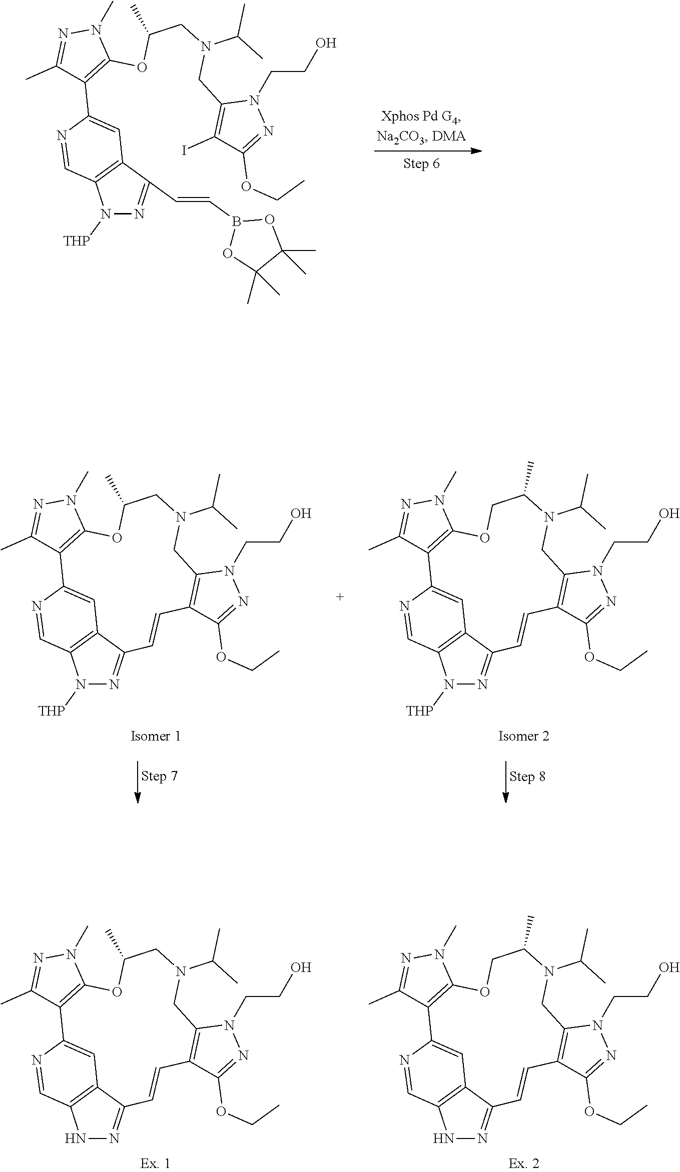

Step 1. To a solution of 5-bromo-3-iodo-1-tetrahydropyran-2-yl-pyrazolo[3,4-c]pyridine, which was prepared according to the method described in Ex. 1, (18.5 g, 45.34 mmol, 1 eq) and commercially available ethynyl(triisopropyl)silane (8.27 g, 45.3 mmol, 1 eq) in DMF (185 mL) was added iodocopper (518 mg, 2.72 mmol, 0.06 eq), TEA (134 g, 1.33 mol, 29 eq) and cyclopentyl(diphenyl)phosphane; dichloropalladium; iron (Pd(dppf)C₂) (3.32 g, 4.53 mmol, 0.1 eq). The mixture was stirred at 25° C. for 24 hr under N₂. On completion, the mixture was quenched with water (200 mL) and extracted with ethyl acetate (50 mL×3). The combined organic phase was dried over anhydrous sodium sulfate, filtered, and concentrated to give a residue. The residue was purified by column chromatography (SiO₂, Petroleum ether/Ethyl acetate=1:0 to 6:1) to give 2-(5-bromo-1-tetrahydropyran-2-yl-pyrazolo[3,4-c]pyridin-3-yl)ethynyl-triisopropyl-silane (10.6 g, 23 mmol, 51% yield) as a white oil. ¹H NMR (400 MHz, DMSO-d₆) δ=9.17 (s, 1H), 7.79 (s, 1H), 6.04 (br d, J=8.4 Hz, 1H), 3.94-3.73 (m, 2H), 2.37-2.24 (m, 1H), 2.02 (br s, 2H), 1.79-1.67 (m, 1H), 1.61 (br d, J=3.6 Hz, 2H), 1.17-1.10 (m, 21H). LCMS: (M+1:464.1).

Step 2. To a solution of 2-(5-bromo-1-tetrahydropyran-2-yl-pyrazolo[3,4-c]pyridin-3-yl)ethynyl-triisopropyl-silane (1.00 g, 2.16 mmol, 1 eq) in NMP (30 mL) was added commercially available 2,5-dimethylpyrazol-3-ol (291 mg, 2.59 mmol, 1.2 eq) and K₂CO₃(598 mg, 4.32 mmol, 2 eq), then [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (73.9 mg, 86.5 μmol, 0.04 eq) was added under N₂. The mixture was stirred at 130° C. for 2 h. On completion, the mixture was quenched with water (20 ml) and extracted with 2-methyl tetrahydrofuran (20 mL×3). The combined organic phase was dried over anhydrous sodium sulfate, filtered, and concentrated to give a residue. The residue was purified by column chromatography (SiO₂, PE/EA=1:0 to 1:1) to give 2,5-dimethyl-4-[1-tetrahydropyran-2-yl-3-(2-triisopropylsilylethynyl)pyrazolo[3,4-c]pyridin-5-yl]pyrazol-3-ol (510 mg, 1.03 mmol, 48% yield) as a brown solid. LCMS: (M+1:494.7).

Step 3. To a solution of [(1R)-2-[[2-[2-[tert-butyl(dimethyl)silyl]oxyethyl]-5-ethoxy-4-iodo-pyrazol-3-yl]methyl-isopropyl-amino]-1-methyl-ethyl]methanesulfonate, which was prepared according to the method described in Ex. 1, (1.02 g, 1.69 mmol, 2 eq) and 2,5-dimethyl-4-[1-tetrahydropyran-2-yl-3-(2-triisopropylsilylethynyl)pyrazolo[3,4-c]pyridin-5-yl]pyrazol-3-ol (416 mg, 842 mol, 1 eq) in DMF (28 mL) was added K₂CO₃ (349 mg, 2.53 mmol, 3 eq). The mixture was stirred at 60° C. for 2 h. On completion, the mixture was quenched with water (50 mL) and extracted with ethyl acetate (25 mL×3). The combined organic phase was dried over anhydrous sodium sulfate, filtered and concentrated to give a residue. The residue was purified by column chromatography (SiO₂, PE:THF=1:0 to 2:1) to give (2S)-N-[[2-[2-[tert-butyl(dimethyl)silyl]oxyethyl]-5-ethoxy-4-iodo-pyrazol-3-yl]methyl]-2-[2,5-dimethyl-4-[1-tetrahydropyran-2-yl-3-(2-triisopropylsilylethynyl) pyrazolo[3,4-c]pyridin-5-yl]pyrazol-3-yl]oxy-N-isopropyl-propan-1-amine (660 mg, 659 μmol, 78% yield) as a yellow solid. LCMS: (M+1:1001.9).

Step 4. To a solution of (2S)-N-[[2-[2-[tert-butyl(dimethyl)silyl]oxyethyl]-5-ethoxy-4-iodo-pyrazol-3-yl]methyl]-2-[2,5-dimethyl-4-[1-tetrahydropyran-2-yl-3-(2-triisopropylsilylethynyl)pyrazolo[3,4-c]pyridin-5-yl]pyrazol-3-yl]oxy-N-isopropyl-propan-1-amine (600 mg, 599 μmol, 1 eq) in DMF (6 mL) was added CsF (637 mg, 4.19 mmol, 7 eq). The mixture was stirred at 50° C. for 1 h. On completion, the mixture was quenched with saturated sodium chloride (12 mL) and extracted with EA (10 mL×3). The combined organic phase was dried over anhydrous sodium sulfate, filtered and concentrated to give a residue. The residue was purified by column chromatography (SiO₂, PE:THF=1:0 to 1:1) to give 2-[3-ethoxy-5-[[[(2S)-2-[4-(3-ethynyl-1-tetrahydropyran-2-yl-pyrazolo[3,4-c]pyridin-5-yl)-2,5-dimethyl-pyrazol-3-yl]oxypropyl]-isopropyl-amino]methyl]-4-iodo-pyrazol-1-yl]ethanol (437 mg, 598 μmol, 100% yield) as a yellow solid. LCMS: (M+1:731.2).

Step 5. The mixture of 2-[3-ethoxy-5-[[[(2S)-2-[4-(3-ethynyl-1-tetrahydropyran-2-yl-pyrazolo[3,4-c]pyridin-5-yl)-2,5-dimethyl-pyrazol-3-yl]oxypropyl]-isopropyl-amino]methyl]-4-iodo-pyrazol-1-yl]ethanol (350 mg, 479 mol, 1 eq) and commercially available 4,4,4′,4′,5,5,5′,5′-octamethyl-2,2′-bi(1,3,2-dioxaborolane (134 mg, 527 μmol, 1.1 eq) in dioxane (7 mL) was added PPh₃ (125 mg, 479 μmol, 1 eq) and Cu₂O (34.3 mg, 239 μmol, 0.5 eq), the resulting mixture was stirred at 60° C. for 14 h under N₂. On completion, the mixture was filtered and concentrated to give a residue. The residue was purified by column chromatography (SiO₂, PE/THF=1:1 to 1:4) to give 2-[5-[[[(2S)-2-[2,5-dimethyl-4-[1-tetrahydropyran-2-yl-3-[(E)-2-(4,4,5,5-tetramethyl-1,3,2-dioxaborolan-2-yl)vinyl]pyrazolo[3,4-c]pyridin-5-yl]pyrazol-3-yl]oxypropyl]-isopropyl-amino]methyl]-3-ethoxy-pyrazol-1-yl]ethanol (440 mg, crude) as a brown solid. LCMS: (M+1:859.7).

Step 6. To a solution of 2-[5-[[[(2S)-2-[2,5-dimethyl-4-[1-tetrahydropyran-2-yl-3-[(E)-2-(4,4,5,5-tetramethyl-1,3,2-dioxaborolan-2-yl)vinyl]pyrazolo[3,4-c]pyridin-5-yl]pyrazol-3-yl]oxypropyl]-isopropyl-amino]methyl]-3-ethoxy-4-iodo-pyrazol-1-yl]ethanol (356 mg, 414 mol, 1 eq) in DMA (4.5 mL) was added Na₂CO₃ (1.5 M, 829 μL, 3 eq) and Xphos Pd G4 (35.6 mg, 41.4 μmol, 0.1 eq). The mixture was stirred at 80° C. for 2 h at N₂ atmosphere. On completion, the mixture was quenched with water (15 mL) and extracted with ethyl acetate (25 mL×3). The combined organic phase was dried over anhydrous sodium sulfate, filtered, and concentrated to give a residue. The residue was purified by column chromatography (SiO₂, PE:THF=1:0 to 1:2) to give 2-[(8S,17E)-15-ethoxy-10-isopropyl-3,5,8-trimethyl-21-tetrahydropyran-2-yl-7-oxa-4,5,10,13,14,20,21,24-octazapentacyclo[17.5.2.0^{2,6}.0^{12,16}.0^{22,26}]hexacosa-1(25),2(6),3,12(16),14,17,19,22(26),23-nonaen-13-yl]ethanol (170 mg, 281 μmol, 68% yield) as a yellow oil [LCMS: (M+1:605.3)] and (S,E)-2-(53-ethoxy-7-isopropyl-11,13,8-trimethyl-11H,21H,51H-10-oxa-7-aza-2(5,3)-pyrazolo[3,4-c]pyridina-1,5(4,5)-dipyrazolacyclodecaphan-3-en-51-yl)ethan-1-ol (40 mg, 66.1 μmol) [LCMS: (M+1:605.4)].

Step 7. To a solution of 2-[(8S,17E)-15-ethoxy-10-isopropyl-3,5,8-trimethyl-21-tetrahydropyran-2-yl-7-oxa-4,5,10,13,14,20,21,24-octazapentacyclo[17.5.2.0^{2,6}.0^{12,16} 0^{22,26}]hexacosa-1(25),2(6),3,12(16),14,17,19,22(26),23-nonaen-13-yl]ethanol (100 mg, 165 μmol, 1 eq) in DCM (1 mL) was added TFA (307 mg, 2.69 mmol, 0.2 mL, 16.3 eq). The mixture was stirred at 25° C. for 0.5 h. On completion, the mixture was concentrated to give a brown oil (86.0 mg, 165 μmol, 99% yield). LCMS: (M+1:617.2).

The brown oil (170 mg, 275 mol, 1 eq) was dissolved in THE (1.7 mL), MeOH (1.7 mL), and H₂O (0.5 mL), and LiOH (19.8 mg, 827 μmol, 3 eq) was added. The mixture was stirred at 25° C. for 1 h. On completion, the mixture was concentrated to give a residue. The crude product was purified by reversed-phase Column chromatography (column: Phenomenex luna C18 150*25 mm*10 um; mobile phase: [water(FA)-ACN]; gradient:12%-43% B over 15 min) to give Ex. 1 (14.12 mg, 27.12 μmol, 9.84% yield) as a yellow solid. LCMS: (M+1: 521.3).

Step 8. To a solution of (S,E)-2-(53-ethoxy-7-isopropyl-11,13,8-trimethyl-11H,21H,51H-10-oxa-7-aza-2(5,3)-pyrazolo[3,4-c]pyridina-1,5(4,5)-dipyrazolacyclodecaphan-3-en-51-yl)ethan-1-ol (40 mg, 66.1 μmol, 1 eq) in DCM (0.5 mL) was added TFA (153 mg, 1.35 mmol, 20.3 eq). The mixture was stirred at 25° C. for 1 h. On completion, the mixture was concentrated to give a residue. The crude product was purified by reversed-phase Column chromatography (column: Phenomenex luna C18 150*25 mm*10 um; mobile phase: [water(FA)-ACN]; gradient:16%-46% B over 15 min) to give Ex. 2 (1.58 mg, 3.03 μmol, 4.59% yield) as a yellow solid. LCMS: (M+1: 521.2). ¹H NMR for Ex. 2 is shown in the below NMR table.

### Preparation of 2-[(10R,17E)-8,10-dimethyl-12-(propan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 3) and 2-[(11S,17E)-8,11-dimethyl-12-(propan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 4)

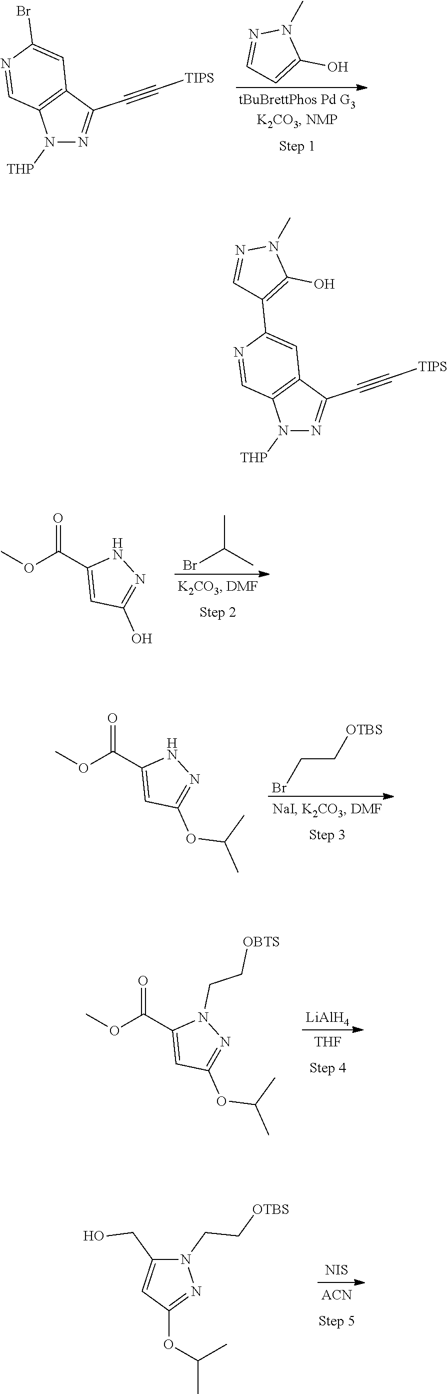

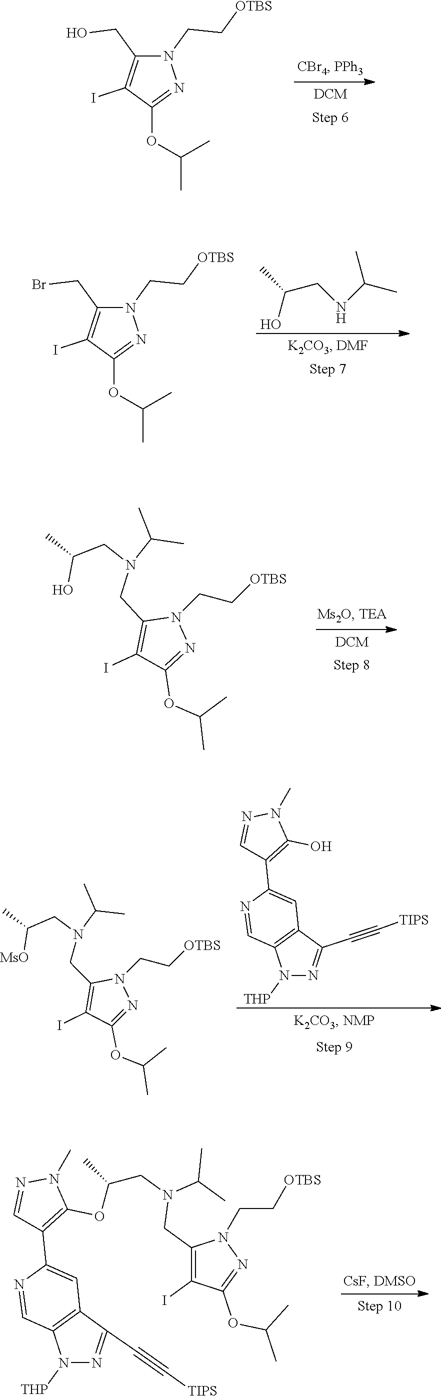

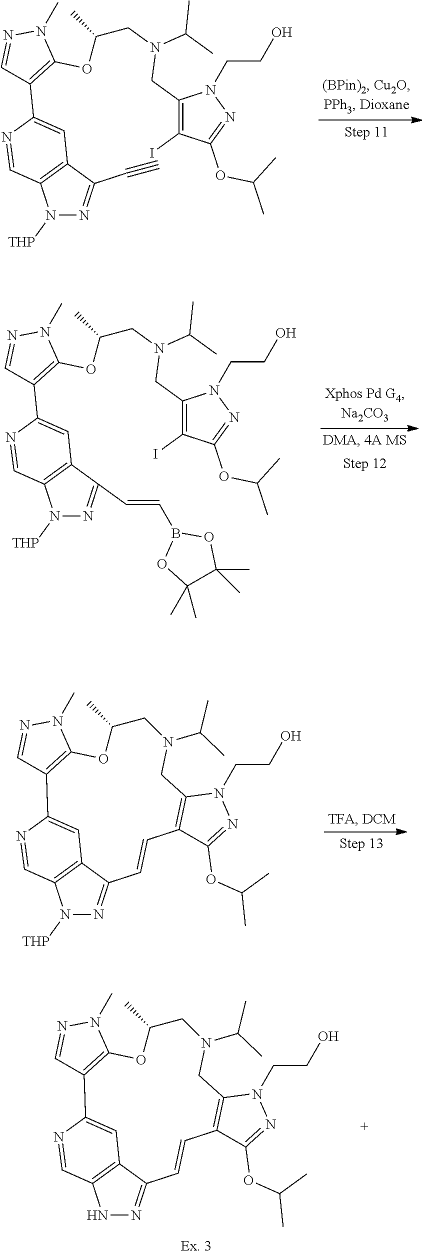

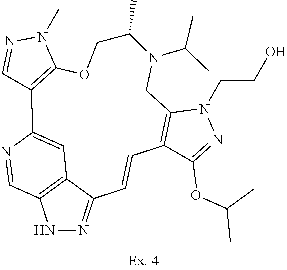 [SMILES: CC(C)Oc1nn(CCO)c2c1/C=C/c1n[nH]c3cnc(cc13)-c1cnn(C)c1OC[C@H](C)N(C(C)C)C2]

Step 1. To a solution of 2-(5-bromo-1-tetrahydropyran-2-yl-pyrazolo[3,4-c]pyridin-3-yl)ethynyl-triisopropyl-silane, which was prepared according to the method described in Ex. 2, (1.50 g, 3.24 mmol, 1 eq,) in NMP (25 mL) was added commercially available 2-methylpyrazol-3-ol (382 mg, 3.89 mmol, 1.2 eq), [2-(2-aminophenyl)phenyl]-methylsulfonyloxy-palladium; ditert-butyl-[3,6-dimethoxy-2-(2,4,6-triisopropylphenyl) phenyl]phosphane (tBuBrettPhos Pd G3) (277 mg, 324 μmol, 0.1 eq) and K₂CO₃ (1.34 g, 9.73 mmol, 3 eq) under N₂, then the mixture was stirred at 130° C. for 1 h. On completion, the reaction mixture was diluted with H₂O (30 mL) and extracted with 2-Me-THF (20 mL×3). The combined organic layers were washed with Brine (20 mL×2), dried over Na₂SO₄, filtered and concentrated under reduced pressure to give 2-methyl-4-[1-tetrahydropyran-2-yl-3-(2-triisopropylsilylethynyl)pyrazolo[3,4-c]pyridin-5-yl]pyrazol-3-ol (1.50 g, crude) as a brown oil. LCMS: (M+1:480.3).

Step 2. To a mixture of commercially available methyl 3-hydroxy-1H-pyrazole-5-carboxylate (3.00 g, 21.1 mmol, 1.00 eq), K₂CO₃ (8.75 g, 63.3 mmol, 3.00 eq) in DMF (50 mL) was added 2-bromopropane (3.12 g, 25.3 mmol, 1.20 eq). The mixture was stirred at 80° C. for 2 h. On completion, the mixture was poured into water (80 mL) aqueous solution, the aqueous phase was extracted with ethyl acetate (40 mL×2). The combined organic phase was washed with brine (30 mL×2), dried over anhydrous Na₂SO₄, filtered and concentrated under vacuum. The residue was purified by flash silica gel chromatography (ISCO®; 40 g SepaFlash® Silica Flash Column, Eluent of 0-30% Ethyl acetate/Petroleum ether gradient at 60 mL/min) to give methyl 3-isopropoxy-1H-pyrazole-5-carboxylate (1.95 g, 10.4 mmol, 50% yield, 99% purity) as a yellow oil. ¹H NMR (400 MHz, CDCl₃) δ=8.03 (s, 1H), 6.20 (s, 1H), 4.74 (d, J=6.0, 12.4 Hz, 1H), 3.91 (s, 3H), 1.37 (d, J=6.2 Hz, 6H). LCMS: (M+1:185.3).

Step 3. To a mixture of methyl 3-isopropoxy-1H-pyrazole-5-carboxylate (1.95 g, 10.5 mmol, 1.00 eq), commercially available 2-bromoethoxy-tert-butyl-dimethyl-silane (3.80 g, 15.9 mmol, 1.50 eq) and K₂CO₃ (4.39 g, 31.7 mmol, 3 eq) in DMF (25 mL) was added NaI (1.59 g, 10.6 mmol, 1 eq). The mixture was stirred at 60° C. for 16 h. On completion, the mixture was poured into sat. NH₄Cl (10 mL) aqueous solution, the aqueous phase was extracted with ethyl acetate (10 mL×2). The combined organic phase was washed with brine (10 mL×2), dried over anhydrous Na₂SO₄, filtered and concentrated under vacuum. The residue was purified by flash silica gel chromatography (ISCO®; 25 g SepaFlash® Silica Flash Column, Eluent of 0-25% Ethyl acetate/Petroleum ether gradient at 50 mL/min) to give methyl 2-[2-[tert-butyl(dimethyl)silyl]oxyethyl]-5-isopropoxy-pyrazole-3-carboxylate (1.50 g, 4.38 mmol, 41% yield) as a yellow oil. ¹H NMR (400 MHz, CDCl₃) δ=6.16 (s, 1H), 4.69 (td, J=6.4, 12.4 Hz, 1H), 4.56 (t, J=5.6 Hz, 2H), 3.89 (t, J=5.6 Hz, 2H), 3.85 (s, 3H), 1.34 (d, J=6.0 Hz, 6H), 0.85-0.79 (m, 9H), −0.06 (s, 6H).

Step 4. To a mixture of methyl 2-[2-[tert-butyl (dimethyl) silyl]oxyethyl]-5-isopropoxy-pyrazole-3-carboxylate (1.50 g, 4.38 mmol, 1.00 eq) in THE (15 mL) was added LiAlH₄ (166 mg, 4.38 mmol, 1.00 eq) at 0° C. The mixture was stirred at 0° C. for 2 h. On completion, the mixture was quenched with MeOH (150 mL) at 0° C., and the mixture was concentrated to give a residue. The residue was purified by flash silica gel chromatography (ISCO®; 12 g SepaFlash® Silica Flash Column, Eluent of 0-15% Ethyl acetate/Petroleum ether gradient at 50 mL/min) to give [2-[2-[tert-butyl (dimethyl) silyl]oxyethyl]-5-isopropoxy-pyrazol-3-yl]methanol (950 mg, 2.93 mmol, 67% yield, 97% purity) as a colorless oil. ¹H NMR (400 MHz, CDCl₃-d) 6=5.61 (s, 1H), 4.67 (td, J=2.0, 6.4, 12.4 Hz, 1H), 4.53 (s, 2H), 4.20-4.09 (m, 2H), 3.96 (t, J=4.8 Hz, 2H), 1.34 (d, J=6.0 Hz, 6H), 0.83 (s, 9H), 0.01 (s, 6H).

Step 5. To a mixture of [2-[2-[tert-butyl(dimethyl)silyl]oxyethyl]-5-isopropoxy-pyrazol-3-yl]methanol (900 mg, 2.86 mmol, 1.00 eq) in ACN (10 mL) was added NIS (708 mg, 3.15 mmol, 1.10 eq) at 0° C. The mixture was stirred at 25° C. for 2 h. On completion, the mixture was quenched with saturate the sodium sulfite solution (10 mL) at 0° C., and extracted with ethyl acetate (10 mL×3). The combined organic phase was dried over anhydrous Na₂SO₄, filtered and the filtrate was concentrated to give a residue. The residue was purified by flash silica gel chromatography (ISCO®; 12 g SepaFlash® Silica Flash Column, Eluent of 0-17% Ethyl acetate/Petroleum ether gradient at 55 mL/min) to give [2-[2-[tert-butyl (dimethyl) silyl]oxyethyl]-4-iodo-5-isopropoxy-pyrazol-3-yl]methanol (1.1 g, 2.47 mmol, 86% yield, 99% purity) as a yellow solid. ¹H NMR (400 MHz, CDCl₃-d) 6=4.90-4.75 (m, 1H), 4.59 (s, 2H), 4.22 (t, J=4.8 Hz, 2H), 3.92 (t, J=4.8 Hz, 2H), 3.21 (s, 1H), 1.36 (d, J=6.2 Hz, 6H), 0.82 (s, 9H), 0.00 (s, 6H). LCMS: (M+1:441.0).

Step 6. To a solution of [2-[2-[tert-butyl(dimethyl)silyl]oxyethyl]-4-iodo-5-isopropoxy-pyrazol-3-yl]methanol (1 g, 2.27 mmol, 1.00 eq), PPh₃ (715 mg, 2.72 mmol, 1.2 eq) in DCM (10 mL) was added CBr₄ (904 mg, 2.72 mmol, 1.20 eq) at 0° C. The mixture was stirred at 25° C. for 1 h. On completion, the mixture was diluted with water (30 mL) and extracted with ethyl acetate (10 mL×3). The combined organic phase was washed with water (20 mL×2) and dried over Na₂SO₄, filtered and the filtrate was concentrated to give a residue. The residue was purified by column chromatography (SiO₂, Petroleum ether/Ethyl acetate=1:1 to 1:1) to give 2-[5-(bromomethyl)-4-iodo-3-isopropoxy-pyrazol-1-yl]ethoxy-tert-butyl-dimethyl-silane (830 mg, 1.65 mmol, 73% yield) as yellow oil. LCMS: (M+1:504.8).

Step 7. To a solution of commercially available (2R)-1-(isopropylamino)propan-2-ol (325 mg, 2.78 mmol, 1 eq) and 2-[5-(bromomethyl)-4-iodo-3-isopropoxy-pyrazol-1-yl]ethoxy-tert-butyl-dimethyl-silane (1.40 g, 2.78 mmol, 1 eq) in DMF (20 mL) was added K₂CO₃ (1.15 g, 8.35 mmol, 3 eq). The mixture was stirred at 25° C. for 14 hours. On completion, the mixture was quenched with water (20 mL) and extracted with ethyl acetate (25 mL×3). The combined organic phase was dried over anhydrous sodium sulfate, filtered, and concentrated to give (2R)-1-[[2-[2-[tert-butyl(dimethyl)silyl]oxyethyl]-4-iodo-5-isopropoxy-pyrazol-3-yl]methyl-isopropyl-amino]propan-2-ol (1.50 g, 2.78 mmol, 99% yield) as colorless oil. ¹H NMR (400 MHz, CDCl3) δ=4.87-4.74 (m, 1H), 4.31-4.09 (m, 2H), 3.93-3.82 (m, 2H), 3.80-3.56 (m, 3H), 2.88 (s, 2H), 2.40-2.23 (m, 2H), 1.37-1.32 (m, 6H), 1.08 (d, J=6.4 Hz, 6H), 1.04 (d, J=6.4 Hz, 3H), 0.81 (s, 9H), −0.04-0.12 (m, 6H). LCMS: (M+1: 540.1).

Step 8. To a solution of (2R)-1-∥2-|2-] tert-butyl(dimethyl)silylloxyethyl]-4-iodo-5-isopropoxy-pyrazol-3-yl]methyl-isopropyl-amino]propan-2-ol (1.80 g, 3.34 mmol, 1 eq) in DCM (20 mL) was added TEA (675 mg, 6.67 mmol, 2 eq) and methylsulfonyl methanesulfonate (581 mg, 3.34 mmol, 1 eq) at 0° C., then the mixture was stirred at 25° C. for 1 h. On completion, the reaction mixture was diluted with 20 mL H₂O and extracted with 30 mL DCM (10 mL×3). The combined organic layers were dried over Na₂SO₄, filtered and concentrated under reduced pressure to give [(1R)-2-[[2-[2-[tert-butyl(dimethyl)silyl]oxyethyl]-4-iodo-5-isopropoxy-pyrazol-3-yl]methyl-isopropyl-amino]-1-methyl-ethyl]methanesulfonate (2.00 g, crude) as yellow oil. LCMS: (M+1:618.4).

Step 9. To a solution of [(1R)-2-[[2-[2-[tert-butyl(dimethyl)silyl]oxyethyl]-4-iodo-5-isopropoxy-pyrazol-3-yl]methyl-isopropyl-amino]-1-methyl-ethyl]methanesulfonate (1.93 g, 3.13 mmol, 1 eq) in NMP (30 mL) was added K₂CO₃ (864 mg, 6.25 mmol, 2 eq) and 2-methyl-4-[1-tetrahydropyran-2-yl-3-(2-triisopropylsilylethynyl)pyrazolo[3,4-c]pyridin-5-yl]pyrazol-3-ol (1.50 g, 3.13 mmol, 1 eq), then the mixture was stirred at 60° C. for 2 hr. On completion, the reaction mixture was diluted with H₂O (30 mL) and extracted with 60 mL 2-Me-THF (20 mL×3). The combined organic layers were dried over Na₂SO₄, filtered and concentrated under reduced pressure to give a residue. The residue was purified by flash silica gel chromatography (ISCO®; 80 g SepaFlash® Silica Flash Column, Eluent of 0-100% Ethyl acetate/Petroleum ether gradient at 50 mL/min) to give (2S)-N-[[2-[2-[tert-butyl(dimethyl)silyl]oxyethyl]-4-iodo-5-isopropoxy-pyrazol-3-yl]methyl]-N-isopropyl-2-[2-methyl-4-[1-tetrahydropyran-2-yl-3-(2-triisopropylsilylethynyl)pyrazolo[3,4-c]pyridin-5-yl]pyrazol-3-yl]oxy-propan-1-amine (1.30 g, 1.30 mmol, 42% yield) as a brown oil. LCMS: (M+1:1002.1).

Step 10. To a solution of (2S)-N-[[2-[2-[tert-butyl(dimethyl)silyl]oxyethyl]-4-iodo-5-isopropoxy-pyrazol-3-yl]methyl]-N-isopropyl-2-[2-methyl-4-[1-tetrahydropyran-2-yl-3-(2-triisopropylsilylethynyl)pyrazolo[3,4-c]pyridin-5-yl]pyrazol-3-yl]oxy-propan-1-amine (1.20 g, 1.20 mmol, 1 eq) in DMSO (10 mL) was added CsF (1.27 g, 8.39 mmol, 7 eq), then the mixture was stirred at 25° C. for 2 h. On completion, the reaction mixture was diluted with H₂O (20 mL) and extracted with 30 mL EA (10 mL×3). The combined organic layers were washed with 30 mL Brine (15 mL×2), dried over Na₂SO₄, filtered and concentrated under reduced pressure to give a residue. The residue was purified by flash silica gel chromatography (ISCO®; 20 g SepaFlash® Silica Flash Column, Eluent of 0-100% Ethyl acetate/Petroleum ether gradient at 50 mL/min) to give 2-[5-[[[(2S)-2-[4-(3-ethynyl-1-tetrahydropyran-2-yl-pyrazolo[3,4-c]pyridin-5-yl)-2-methyl-pyrazol-3-yl]oxypropyl]-isopropyl-amino]methyl]-4-iodo-3-isopropoxy-pyrazol-1-yl]ethanol (700 mg, 958 mol, 80% yield) as a white solid. ¹H NMR (400 MHz, DMSO-d₆) δ=9.24 (s, 1H), 7.88 (d, J=3.2 Hz, 1H), 7.82 (s, 1H), 6.10-5.89 (m, 1H), 4.76 (br d, J=4.4 Hz, 1H), 4.72-4.68 (m, 1H), 4.68-4.60 (m, 1H), 4.34-4.21 (m, 1H), 4.21-4.08 (m, 1H), 4.08-3.93 (m, 2H), 3.87 (br d, J=10.8 Hz, 1H), 3.79 (br s, 2H), 3.68 (s, 1H), 3.62 (s, 2H), 3.57 (br s, 3H), 2.93-2.72 (m, 1H), 2.69 (s, 2H), 2.39-2.24 (m, 1H), 2.09-1.95 (m, 3H), 1.82-1.66 (m, 1H), 1.61 (br s, 2H), 1.26-1.18 (m, 7H), 1.04-0.91 (m, 6H). LCMS: (M+1:731.4).

Step 11. To a solution of 2-[5-[[[(2S)-2-[4-(3-ethynyl-1-tetrahydropyran-2-yl-pyrazolo[3,4-c]pyridin-5-yl)-2-methyl-pyrazol-3-yl]oxypropyl]-isopropyl-amino]methyl]-4-iodo-3-isopropoxy-pyrazol-1-yl]ethanol (650 mg, 890 μmol, 1 eq) in dioxane (10 mL) was added Cu₂O (63.7 mg, 445 mol, 0.5 eq), PPh₃ (233 mg, 889 mol, 1 eq) and BPD (452 mg, 1.78 mmol, 2 eq) under N₂, then the mixture was stirred at 70° C. for 2 h. On completion, the reaction mixture was concentrated under reduced pressure to give a residue. The residue was purified by flash silica gel chromatography (ISCO®; 20 g SepaFlash® Silica Flash Column, Eluent of 0-80% THF/Petroleum ether gradient at 50 mL/min) to give 2-[4-iodo-3-isopropoxy-5-[[isopropyl-[(2S)-2-[2-methyl-4-[1-tetrahydropyran-2-yl-3-[(E)-2-(4,4,5,5-tetramethyl-1,3,2-dioxaborolan-2-yl)vinyl]pyrazolo[3,4-c]pyridin-5-yl]pyrazol-3-yl]oxypropyl]amino]methyl]pyrazol-1-yl]ethanol (680 mg, 792 mol, 89% yield) as a yellow solid. LCMS: (M+1:859.6).

Step 12. To a solution of 2-[4-iodo-3-isopropoxy-5-[[isopropyl-[(2S)-2-[2-methyl-4-[1-tetrahydropyran-2-yl-3-[(E)-2-(4,4,5,5-tetramethyl-1,3,2-dioxaborolan-2-yl)vinyl]pyrazolo[3,4-c]pyridin-5-yl]pyrazol-3-yl]oxypropyl]amino]methyl]pyrazol-1-yl]ethanol (600 mg, 699 μmol, 1 eq) in DMA (7 mL) was added Na₂CO₃ (1.5 M, 1.40 mL, 3 eq) and dicyclohexyl-[2-(2,4,6-triisopropylphenyl)phenyl]phosphane; methanesulfonate; [2-[2-(methylamino)phenyl]phenyl]palladium(1+) (Xphos Pd G4) (60.0 mg, 70 μmol, 0.1 eq) under N₂, then the mixture was stirred at 80° C. for 2 h. On completion, the reaction mixture was diluted with 20 mL H₂O and extracted with 30 mL EA (10 mL×3). The combined organic layers were washed with 20 mL Brine (10 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 0-70% THE/Petroleum ether gradient at 50 mL/min) to give 2-[(10R,17E)-8,10-dimethyl-2-(oxan-2-yl)-12-(propan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (380 mg, 628 μmol, 90% yield) as a yellow solid. LCMS: (M+1:605.5).

Step 13. To a solution of 2-[(10R,17E)-8,10-dimethyl-2-(oxan-2-yl)-12-(propan-2-yl)-16-[(propan-2-yl)oxy]-2,8,10,11,12,13-hexahydro-14H-5,3-(azenometheno)tripyrazolol3,4-f:3′,4′-j:4″,3″-n][1,4]oxazacyclopentadecin-14-yl]ethan-1-ol (70.0 mg, 116 μmol, 1 eq) in DCM (1 mL) was added TFA (1.07 g, 9.42 mmol, 81 eq), then the mixture was stirred at 20° C. for 2 h. On completion, the reaction mixture was concentrated under reduced pressure to give a residue. The residue was purified by prep-HPLC (column: Welch Xtimate C18 150*25 mm*5 um; mobilephase: [water(TFA)-ACN]; gradient:13%-43% B over 10 min) to give Ex. 3 (9.71 mg, 18.8 mol, 16% yield) as a yellow solid. LCMS: (M+1:521.3). ¹H NMR for Ex. 3 is shown in the below NMR table.

A byproduct was isolated as Ex. 4 (43.92 mg, 84.5 mol, 18% yield) as a yellow solid. LCMS: (M+1:521.3). ¹H NMR for Ex. 4 is shown in the below NMR table.

### Preparation of 2-[(10S,17E)-12-ethyl-8,10-dimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 9) and 2-[(10R,17E)-12-ethyl-8,10-dimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 10)

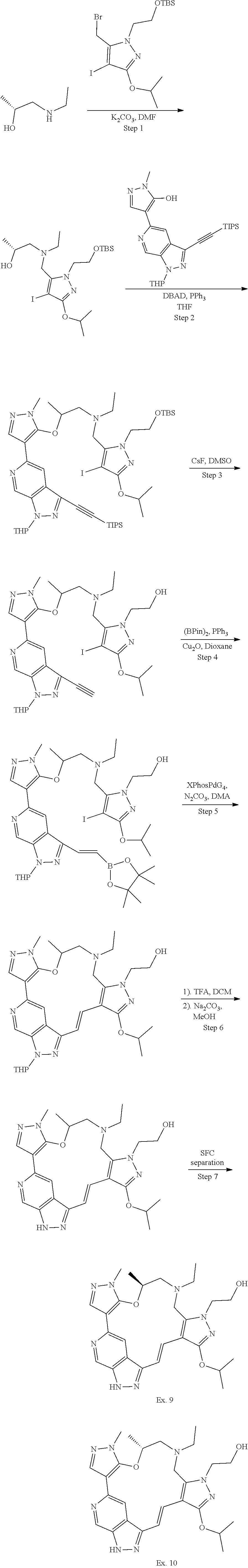

Step 1. To a solution of commercially available (2R)-1-(ethylamino)propan-2-ol (369 mg, 3.58 mmol, 1.2 eq.) in DMF (20 mL) was added 2-[5-(bromomethyl)-4-iodo-3-isopropoxy-pyrazol-1-yl]ethoxy-tert-butyl-dimethyl-silane, which was prepared according to the method described in Ex. 3/Ex. 4, (1.5 g, 2.98 mmol, 1 eq. and K₂CO₃ (1.24 g, 8.94 mmol, 3 eq), then the mixture was stirred at 60° C. for 2 h. On completion, the reaction mixture was diluted with 40 mL H₂O and extracted with 30 mL EA (10 mL×3). The combined organic layers were washed with 20 mL Brine (10 mL×2), dried over Na₂SO₄, filtered, and concentrated under reduced pressure to give a residue. The residue was purified by flash silica gel chromatography (ISCO®; 20 g SepaFlash® Silica Flash Column, Eluent of 0-20% Ethyl acetate/Petroleum ether gradient at 40 mL/min). Compound (2R)-1-[[2-[2-[tert-butyl(dimethyl)silyl]oxyethyl]-4-iodo-5-isopropoxy-pyrazol-3-yl]methyl-ethyl-amino]propan-2-ol (1.5 g, 2.85 mmol, 96% yield) was obtained as a colorless oil. LCMS: (M+1:526.2).

Step 2. To a solution of (2R)-1-[[2-[2-[tert-butyl(dimethyl)silyl]oxyethyl]-4-iodo-5-isopropoxy-pyrazol-3-yl]methyl-ethyl-amino]propan-2-ol (1.10 g, 2.09 mmol, 1 eq) and 2-methyl-4-[1-tetrahydropyran-2-yl-3-(2-triisopropylsilylethynyl)pyrazolo[3,4-c]pyridin-5-yl]pyrazol-3-ol, which was prepared according to the method described in Ex. 3/Ex. 4, (2.01 g, 4.19 mmol, 2 eq.), DBAD (723 mg, 3.14 mmol, 1.5 eq), PPh₃ (1.10 g, 4.19 mmol, 2 eq) in THE (20 mL), then the mixture was stirred at 25° C. for 2h. On completion, the reaction mixture was concentrated under reduced pressure to give a residue. The residue was purified by flash silica gel chromatography (ISCO®; 40 g SepaFlash® Silica Flash Column, Eluent of 0-100% Ethyl acetate/Petroleum ether gradient at 40 mL/min) to give compound N-({1-(2-{[tert-butyl(dimethyl)silyl]oxy}ethyl)-4-iodo-3-[(propan-2-yl)oxy]-1H-pyrazol-5-yl}methyl)-N-ethyl-2-({1-methyl-4-[1-(oxan-2-yl)-3-{[tri(propan-2-yl)silyl]ethynyl}-1H-pyrazolo[3,4-c]pyridin-5-yl]-1H-pyrazol-5-yl}oxy)propan-1-amine (1.60 g, 1.62 mmol, 77% yield) as a yellow oil. LCMS: (M+1:987.4).

Step 3. To a solution of N-({1-(2-{[tert-butyl(dimethyl)silyl]oxy}ethyl)-4-iodo-3-[(propan-2-yl)oxy]-1H-pyrazol-5-yl}methyl)-N-ethyl-2-({1-methyl-4-[1-(oxan-2-yl)-3-{[tri(propan-2-yl)silyl]ethynyl}-1H-pyrazolo[3,4-c]pyridin-5-yl]-1H-pyrazol-5-yl}oxy)propan-1-amine (1.40 g, 1.42 mmol, 1 eq) in DMSO (15 mL) was added CsF (1.29 g, 8.51 mmol, 6 eq), then the mixture was stirred at 25° C. for 1 h. On completion, the reaction mixture was diluted with H₂O (20 mL) and extracted with 30 mL EA (10 mL×3). The combined organic layers were washed with 20 mL Brine (10 mL×2), dried over Na₂SO₄, filtered and concentrated under reduced pressure to give a residue. The residue was purified by flash silica gel chromatography (ISCO®; 20 g SepaFlash® Silica Flash Column, Eluent of 0-50% THE/Petroleum ether gradient at 40 mL/min). Compound 2-{5-({ethyl[2-({4-[3-ethynyl-1-(oxan-2-yl)-1H-pyrazolo[3,4-c]pyridin-5-yl]-1-methyl-1H-pyrazol-5-yl}oxy)propyl]amino}methyl)-4-iodo-3-[(propan-2-yl)oxy]-1H-pyrazol-1-yl}ethan-1-ol (700 mg, 977 μmol, 69% yield) was obtained as a yellow oil. ¹H NMR (400 MHz, DMSO-d₆) δ=9.25 (s, 1H), 7.88 (d, J=2.0 Hz, 1H), 7.82 (s, 1H), 6.07-5.94 (m, 1H), 4.80 (t, J=5.2 Hz, 1H), 4.69 (s, 1H), 4.53-4.42 (m, 1H), 4.11-3.98 (m, 2H), 3.91-3.84 (m, 1H), 3.83-3.74 (m, 1H), 3.66-3.61 (m, 3H), 3.60-3.55 (m, 2H), 3.56-3.55 (m, 1H), 2.82-2.71 (m, 1H), 2.64-2.57 (m, 1H), 2.37-2.27 (m, 1H), 2.18 (s, 2H), 2.08-1.95 (m, 2H), 1.80-1.70 (m, 2H), 1.60 (br s, 2H), 1.29-1.17 (m, 7H), 1.09 (d, J=6.0 Hz, 3H), 0.98-0.90 (m, 3H). LCMS: (M+1:717.4).

Step 4. To a solution of 2-{5-({ethyl[2-({4-[3-ethynyl-1-(oxan-2-yl)-1H-pyrazolo[3,4-c]pyridin-5-yl]-1-methyl-1H-pyrazol-5-yl}oxy)propyl]amino}methyl)-4-iodo-3-[(propan-2-yl)oxy]-1H-pyrazol-1-yl}ethan-1-ol (650 mg, 907 μmol, 1 eq) in dioxane (10 mL) was added Cu₂O (64.9 mg, 454 μmol, 0.5 eq), PPh₃ (238 mg, 907 μmol, 1 eq) and BPD (253 mg, 998 μmol, 1.1 eq) under N₂, then the mixture was stirred at 80° C. for 12 h. On completion, the reaction mixture was concentrated under reduced pressure to give a residue. The residue was purified by flash silica gel chromatography (ISCO®; 20 g SepaFlash® Silica Flash Column, Eluent of 0-65% THF/Petroleum ether gradient at 40 mL/min). Compound 2-{5-[(ethyl{2-[(1-methyl-4-{1-(oxan-2-yl)-3-[(E)-2-(4,4,5,5-tetramethyl-1,3,2-dioxaborolan-2-yl)ethenyl]-1H-pyrazolo[3,4-c]pyridin-5-yl}-1H-pyrazol-5-yl)oxy]propyl}amino)methyl]-4-iodo-3-[(propan-2-yl)oxy]-1H-pyrazol-1-yl}ethan-1-ol (710 mg, 840 μmol, 93% yield) was obtained as a yellow oil. LCMS: (M+1:845.2).

Step 5. To a solution of 2-{5-[(ethyl{2-[(1-methyl-4-{1-(oxan-2-yl)-3-[(E)-2-(4,4,5,5-tetramethyl-1,3,2-dioxaborolan-2-yl)ethenyl]-1H-pyrazolo[3,4-c]pyridin-5-yl}-1H-pyrazol-5-yl)oxy]propyl}amino)methyl]-4-iodo-3-[(propan-2-yl)oxy]-1H-pyrazol-1-yl}ethan-1-ol (650 mg, 770 μmol, 1 eq) in DMA (8 mL) was added Na₂CO3 (1.5 M, 1.5 mL, 3 eq) and dicyclohexyl-[2-(2,4,6-triisopropylphenyl)phenyl]phosphane; methanesulfonate; [2-[2-(methylamino) phenyl]phenyl]palladium(1+) (XPhos Pd G4) (66 mg, 77 μmol, 0.1 eq) under N₂, then the mixture was stirred at 80° C. for 2 h. On completion, the reaction mixture was diluted with 15 mL H₂O and extracted with 30 mL EA (10 mL×3). The combined organic layers were washed with 20 mL Brine (10 mL×2), dried over Na₂SO₄, filtered, and concentrated under reduced pressure to give a residue. Compound 2-[(17E)-12-ethyl-8,10-dimethyl-2-(oxan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (430 mg, 728 mol, 95% yield) was obtained as yellow oil. LCMS: (M+1:591.5).

Step 6. To a solution of 2-[(17E)-12-ethyl-8,10-dimethyl-2-(oxan-2-yl)-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (380 mg, 643 mol, 1 eq) in DCM (4 mL) was added TFA (1.54 g, 13.5 mmol, 21 eq), then the mixture was stirred at 25° C. for 2 h. On completion, the reaction mixture was concentrated under reduced pressure to give a yellow oil (400 mg, crude). LCMS: (M+1:603.1). This yellow oil (400 mg, 664 μmol, 1 eq) was dissolved in MeOH (5 mL) and Na₂CO3 (352 mg, 3.32 mmol, 5 eq) was added. The mixture was stirred at 25° C. for 0.1 h. On completion, the reaction mixture was concentrated under reduced pressure to give a residue. The residue was purified by prep-HPLC (column: Phenomenex Luna C18 200*40 mm*10 um; mobile phase: [water(TFA)-ACN]; gradient:8%-38% B over 10 min). Compound 2-[(17E)-12-ethyl-8,10-dimethyl-16-[(propan-2-yl)oxy]-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (37.1 mg, 73 μmol, 11% yield) was obtained as an orange solid. ¹H NMR (400 MHz, MeOD-d₄) δ=9.12 (s, 1H), 8.77 (s, 1H), 8.10 (s, 1H), 7.62 (br d, J=16.4 Hz, 1H), 7.49-7.37 (m, 1H), 5.38 (br s, 1H), 5.17-5.03 (m, 1H), 4.79 (br s, 2H), 4.53 (br d, J=15.2 Hz, 1H), 4.31-4.14 (m, 2H), 4.01-3.93 (m, 1H), 3.92-3.84 (m, 2H), 3.81 (s, 3H), 3.45 (br d, J=14.4 Hz, 2H), 3.27 (br d, J=6.8 Hz, 1H), 1.50-1.42 (m, 9H), 1.25 (d, J=6.4 Hz, 3H). LCMS: (M+1:507.1).

Step 7. HPLC and SFC separation: the product above was purified by prep-HPLC (column: Welch Xtimate C18 150*25 mm*5 um; mobile phase: [water(TFA)-ACN]; gradient:7%-37% B over 10 min to give a white solid (30 mg, yield 99%), which was further separated by SFC (column: DAICEL CHIRALCEL OD(250 mm*30 mm, 10 um); mobile phase: [C02-MeOH(0.1% NH3H2O)]; B %:35%, isocratic elution mode). Ex. 9 (1.32 mg, 2.61 μmol, 3.56% yield) and Ex. 10 (1.45 mg, 2.86 μmol, 3.91% yield) were obtained as an orange solid.

Ex. 9: LCMS: (M+1:507.3). ¹H NMR for Ex. 9 is shown in the below NMR table.

Ex. 10: LCMS: (M+1:507.3). ¹H NMR for Ex. 10 is shown in the below NMR table.

### Preparation of 2-[(10S,17E)-12-cyclopropyl-16-ethoxy-8,10-dimethyl-2,8,10,11,12,13-hexahydro-14H-5,3-(azenometheno)tripyrazolo[3,4-f:3,4′-j:4″,3″-n][1,4]oxazacyclopentadecin-14-yl]ethan-1-ol (Ex. 30)

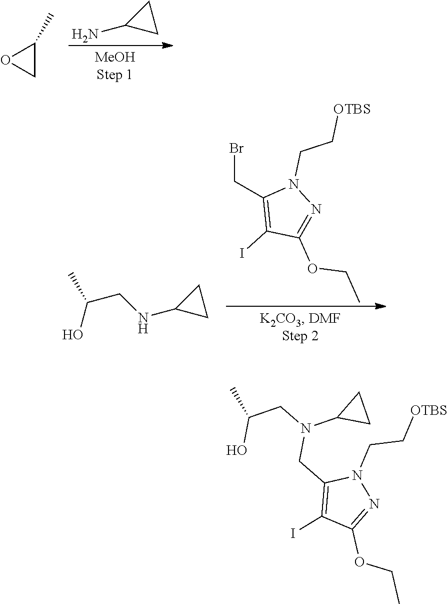

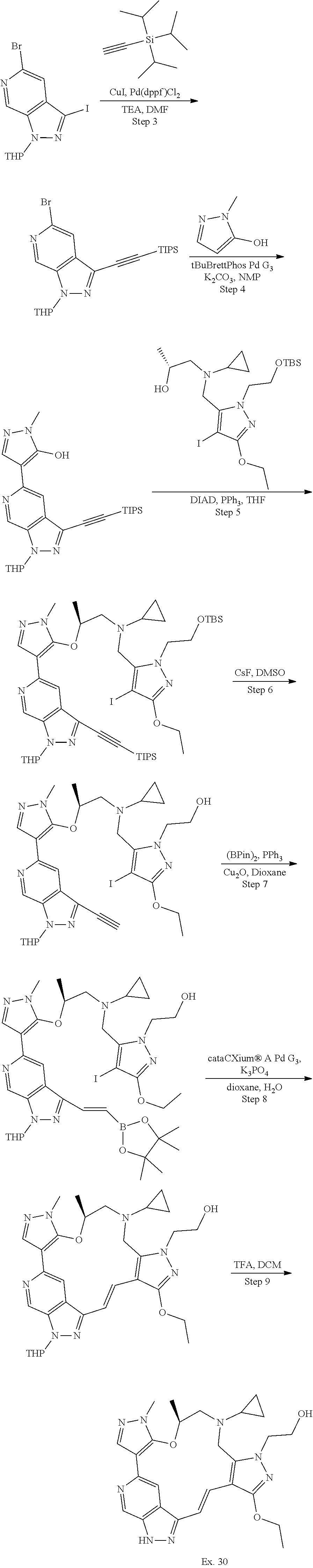

Step 1. To a solution of commercially available (2R)-2-methyloxirane (1.00 g, 17.2 mmol, 1 eq) in MeOH (15 mL) was added commercially available cyclopropanamine (2.95 g, 51.6 mmol, 3 eq). The mixture was stirred at 25° C. for 14 hours. On completion, the mixture was concentrated to give (2R)-1-(cyclopropylamino) propan-2-ol (1.76 g, 15.2 mmol, 88% yield) as colorless oil. ¹H NMR (400 MHz, CDCl₃) δ=3.84-3.71 (m, 1H), 2.87-2.78 (m, 1H), 2.60-2.42 (m, 2H), 2.19-2.09 (m, 1H), 1.15 (d, J=6.0 Hz, 3H), 0.51-0.41 (m, 2H), 0.37-0.27 (m, 2H).

Step 2. To a solution of (2R)-1-(cyclopropylamino) propan-2-ol (500 mg, 4.34 mmol, 1 eq) and 5-(bromomethyl)-1-(2-((tert-butyldimethylsilyl)oxy)ethyl)-3-ethoxy-4-iodo-1H-pyrazole, which was prepared according to the method described in Ex. 1, (2.12 g, 4.34 mmol, 1 eq) in DMF (20 mL) was added K₂CO₃ (1.80 g, 13.0 mmol, 3 eq). The mixture was stirred at 25° C. for 14 hours. On completion, the mixture was quenched with water (50 mL) and extracted with ethyl acetate (30 mL×3). The combined organic phase was dried over anhydrous sodium sulfate, filtered and concentrated to give (2R)-1-[[2-[2-[tert-butyl(dimethyl) silyl]oxyethyl]-5-ethoxy-4-iodo-pyrazol-3-yl]methyl-cyclopropyl-amino]propan-2-ol (2.00 g, 3.82 mmol, 88% yield) as yellow oil. ¹H NMR (400 MHz, CDCl3) δ=4.28-4.22 (m, 2H), 4.20-4.06 (m, 2H), 3.92 (d, J=14.0 Hz, 1H), 3.88-3.84 (m, 2H), 3.68 (d, J=13.6 Hz, 1H), 2.99-2.85 (m, 2H), 2.63-2.43 (m, 2H), 1.94-1.82 (m, 1H), 1.45-1.36 (m, 3H), 1.09 (d, J=6.0 Hz, 3H), 0.82 (s, 9H), 0.56-0.27 (m, 4H), −0.03-0.11 (m, 5H). LCMS: (M+1: 524.6).

Step 3. To a solution of 5-bromo-3-iodo-1-tetrahydropyran-2-yl-pyrazolo[3,4-c]pyridine, which was prepared according to the method described in Ex. 1 (18.5 g, 45.34 mmol, 1 eq), and commercially available ethynyl(triisopropyl)silane (8.27 g, 45.3 mmol, 1 eq) in DMF (185 mL) was added iodocopper (518 mg, 2.72 mmol, 0.06 eq), TEA (134 g, 1.33 mol, 29 eq) and cyclopentyl(diphenyl)phosphane; dichloropalladium; iron (Pd(dppf)Cl₂) (3.32 g, 4.53 mmol, 0.1 eq). The mixture was stirred at 25° C. for 24 hr under N₂. On completion, the mixture was quenched with water (200 mL) and extracted with ethyl acetate (50 mL×3). The combined organic phase was dried over anhydrous sodium sulfate, filtered and concentrated to give a residue. The residue was purified by column chromatography (SiO₂, Petroleum ether/Ethyl acetate=1:0 to 6:1) to give 2-(5-bromo-1-tetrahydropyran-2-yl-pyrazolo[3,4-c]pyridin-3-yl)ethynyl-triisopropyl-silane (10.6 g, 23 mmol, 51% yield) as a white oil. ¹H NMR (400 MHz, DMSO-d₆) δ=9.17 (s, 1H), 7.79 (s, 1H), 6.04 (br d, J=8.4 Hz, 1H), 3.94-3.73 (m, 2H), 2.37-2.24 (m, 1H), 2.02 (br s, 2H), 1.79-1.67 (m, 1H), 1.61 (br d, J=3.6 Hz, 2H), 1.17-1.10 (m, 21H). LCMS: (M+1:464.1).

Step 4. To a solution of 2-(5-bromo-1-tetrahydropyran-2-yl-pyrazolo[3,4-c]pyridin-3-yl)ethynyl-triisopropyl-silane (1.50 g, 3.24 mmol, 1 eq,) in NMP (25 mL) was added commercially available 2-methylpyrazol-3-ol (382 mg, 3.89 mmol, 1.2 eq), [2-(2-aminophenyl)phenyl]-methylsulfonyloxy-palladium; ditert-butyl-[3,6-dimethoxy-2-(2,4,6-triisopropylphenyl) phenyl]phosphane (tBuBrettPhos Pd G3) (277 mg, 324 mol, 0.1 eq) and K₂CO₃ (1.34 g, 9.73 mmol, 3 eq) under N₂. Then the mixture was stirred at 130° C. for 1 h. On completion, the reaction mixture was diluted with H₂O (30 mL) and extracted with 2-Me-THF (20 mL×3). The combined organic layers were washed with Brine (20 mL×2), dried over Na₂SO₄, filtered, and concentrated under reduced pressure to give 2-methyl-4-[1-tetrahydropyran-2-yl-3-(2-triisopropylsilylethynyl)pyrazolo [3,4-c]pyridin-5-yl]pyrazol-3-ol (1.50 g, crude) as a brown oil. LCMS: (M+1:480.3).

Step 5. A mixture of (2R)-1-[[2-[2-[tert-butyl(dimethyl) silyl]oxyethyl]-5-ethoxy-4-iodo-pyrazol-3-yl]methyl-cyclopropyl-amino]propan-2-ol (750 mg, 1.43 mmol, 1 eq), 2-methyl-4-[1-tetrahydropyran-2-yl-3-(2-triisopropylsilylethynyl)pyrazolo[3,4-c]pyridi-5-yl]pyrazol-3-ol (824 mg, 1.72 mmol, 1.2 eq), DIAD (434 mg, 2.15 mmol, 1.5 eq), PPh₃ (751 mg, 2.87 mmol, 2 eq) in THE (10 mL) was degassed and purged with N₂ for 3 times, and then the mixture was stirred at 25° C. for 2 hours under N₂ atmosphere. On completion, the mixture was concentrated to give a residue. The residue was purified by column chromatography (SiO₂, Petroleum ether/Ethyl acetate=1:0 to 1:1) to give N-[[2-[2-[tert-butyl(dimethyl)silyl]oxyethyl]-5-ethoxy-4-iodo-pyrazol-3-yl]methyl]-N-[(2S)-2-[2-methyl-4-[1-tetrahydropyran-2-yl-3-(2-triisopropylsilyl ethynyl) pyrazolo[3,4-c]pyridin-5-yl]pyrazol-3-yl]oxypropyl]cyclopropanamine (730 mg, 740 mol, 51% yield) as a brown solid. LCMS: (M+1: 985.8).

Step 6. To a solution of N-[[2-[2-[tert-butyl(dimethyl) silyl]oxyethyl]-5-ethoxy-4-iodo-pyrazol-3-yl]methyl]-N-[(2S)-2-[2-methyl-4-[1-tetrahydropyran-2-yl-3-(2-triisopropyl silylethynyl)pyrazolo[3,4-c]pyridin-5-yl]pyrazol-3-yl]oxypropyl]cyclopropanamine (730 mg, 740 mol, 1 eq) in DMSO (10 mL) was added CsF (675 mg, 4.45 mmol, 6 eq). The mixture was stirred at 25° C. for 14 hours. On completion, the mixture was quenched with water (80 mL) and extracted with ethyl acetate (50 mL×3). The combined organic phase was dried over anhydrous sodium sulfate, filtered and concentrated to give 2-[5-[[cyclopropyl-[(2S)-2-[4-(3-ethynyl-1-tetrahydropyran-2-yl-pyrazolo[3,4-c]pyridin-5-yl)-2-methyl-pyrazol-3-yl]oxypropyl]amino]methyl1]-3-ethoxy-4-iodo-pyrazol-1-yl]ethanol (500 mg, 699 mol, 94% yield) as a green solid. LCMS: (M+1: 715.3).

Step 7. A mixture of 2-[5-[[cyclopropyl-[(2S)-2-[4-(3-ethynyl-1-tetrahydropyran-2-yl-pyrazolo[3,4-c]pyridin-5-yl)-2-methyl-pyrazol-3-yl]oxypropyl]amino]methyl]-3-ethoxy-4-iodo-pyrazol-1-yl]ethanol (300 mg, 419 mol, 1 eq), commercially available 4,4,4′,4′,5,5,5′,5′-octamethyl-2,2′-bi-1,3,2-dioxaborolane (117 mg, 461 μmol, 1.1 eq), Cu₂O (30.0 mg, 209 μmol, 0.5 eq), PPh₃ (110 mg, 419 mol, 1 eq) in dioxane (5 mL) was degassed and purged with N₂ for 3 times, and then the mixture was stirred at 80° C. for 14 hours under N₂ atmosphere. On completion, the mixture was filtered and concentrated to give a residue. The residue was purified by column chromatography (SiO₂, petroleum ether/THF=1:0 to 0:1) to give 2-[5-[[cyclopropyl-[(2S)-2-[2-methyl-4-[1-tetrahydropyran-2-yl-3-[(E)-2-(4,4,5,5-tetramethyl-1,3,2-dioxaborolan-2-yl) vinyl]pyrazolo[3,4-c]pyridin-5-yl]pyrazol-3-yl]oxypropyl]amino]methyl]-3-ethoxy-4-iodo-pyrazol-1-yl]ethanol (330 mg, 391 mol, 93% yield) as yellow oil. LCMS: (M+1: 843.1).

Step 8. A mixture of 2-[5-[[cyclopropyl-[(2S)-2-[2-methyl-4-[1-tetrahydropyran-2-yl-3-[(E)-2-(4,4,5,5-tetramethyl-1,3,2-dioxaborolan-2-yl)vinyl]pyrazolo[3,4-c]pyridin-5-yl]pyrazol-3-yl]oxypropyl]amino]methyl]-3-ethoxy-4-iodo-pyrazol-1-yl]ethanol (290 mg, 344 mol, 1 eq), [2-(2-aminophenyl)phenyl]palladium(1+); bis(1-adamantyl)-butyl-phosphane; methanesulfonate (cataCXium® A Pd G3) (25.0 mg, 34.4 mol, 0.1 eq), K₃PO₄ (219 mg, 1.03 mmol, 3 eq) in dioxane (5 ml) and H₂O (1 ml) was degassed and purged with N₂ for 3 times, and then the mixture was stirred at 100° C. for 0.5 hours under N₂ atmosphere. On completion, the mixture was filtered and concentrated to give a residue. The residue was purified by column chromatography (SiO₂, Petroleum ether/THF=1:0 to 0:1) to give 2-[(10S,17E)-12-cyclopropyl-16-ethoxy-8,10-dimethyl-2-(oxan-2-yl)-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (120 mg, 203 mol, 59% yield) as a yellow solid. LCMS: (M+1: 589.2).

Step 9. To a solution of 2-[(8S,17E)-10-cyclopropyl-15-ethoxy-5,8-dimethyl-21-tetrahydropyran-2-yl-7-oxa-4,5,10,13,14,20,21,24-octazapentacyclo[17.5.2.02,6.012, 16.022,26]hexacosa-1(25),2(6),3,12(16),14,17,19,22(26),23-nonaen-13-yl]ethanol (100 mg, 169 mol, 1 eq) in DCM (2 mL) was added TFA (193 mg, 1.70 mmol, 10 eq). The mixture was stirred at 25° C. for 0.1 hours. On completion, the mixture was filtered and concentrated to give a residue. The residue was purified by prep-HPLC (column: Phenomenex luna C18 150*25 mm*10 um; mobile phase: [water(FA)-ACN]; gradient:20%-50% B over 15 min) to give Ex. 30 (12.52 mg, 22.3 mol, 13% yield, 98% purity, FA) as a yellow solid. LCMS: (M+1: 505.2). ¹H NMR for Ex. 30 is shown in the below NMR table.

### Preparation of 1-f[(10S,17E)-16-ethoxy-6,8,10,12-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]methyl}cyclopropan-1-ol (Ex. 129)

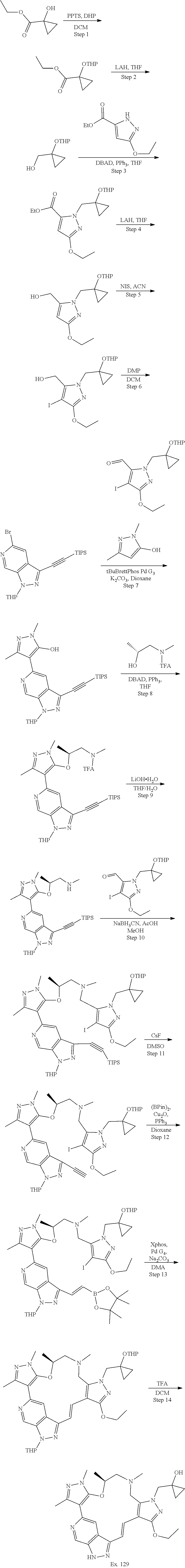

Step 1. To a solution of commercially available ethyl 1-hydroxycyclopropanecarboxylate (5.00 g, 38.4 mmol, 1 eq) in DCM (100 mL) was added PPTS (965 mg, 3.84 mmol, 0.1 eq) and DHP (6.46 g, 76.8 mmol, 7.03 mL, 2 eq), and the mixture was stirred at 25° C. for 16 h. On completion, the reaction mixture was diluted with H₂O (200 mL) and extracted with DCM (80 mL×3). The combined organic layers were washed with brine (100 mL×2), dried over Na₂SO₄, filtered, and concentrated under reduced pressure to give a residue. The residue was purified by flash silica gel chromatography to give ethyl 1-tetrahydropyran-2-yloxycyclopropanecarboxylate (8.20 g, 99% yield) as colorless oil. ¹H NMR (400 MHz, DMSO-d₆) δ=4.87-4.79 (m, 1H), 4.19-4.02 (m, 2H), 3.80-3.68 (m, 1H), 3.49-3.37 (m, 1H), 1.77-1.63 (m, 2H), 1.59-1.40 (m, 5H), 1.25-1.07 (m, 6H). LCMS: (M+23:237.2).

Step 2. To a solution of ethyl 1-tetrahydropyran-2-yloxycyclopropanecarboxylate (3.00 g, 14.0 mmol, 1 eq) in THE (30 mL) was added LiAlH₄ (2.5 M, 11 mL, 2 eq) at 0° C., and the mixture was stirred at 0° C. under N₂ for 1 h. On completion, the reaction mixture was quenched by addition sat. NH₄Cl (30 mL) at 0° C., and then diluted with H₂O (70 mL) and extracted with EA (30 mL×3). The combined organic layers were washed with brine (30 mL×2), dried over Na₂SO₄, filtered, and concentrated under reduced pressure to give (1-tetrahydropyran-2-yloxycyclopropyl)methanol (1.45 g, 60% yield) as a colorless oil. ¹H NMR (400 MHz, DMSO-d₆) δ=4.91-4.70 (m, 1H), 4.65-4.43 (m, 1H), 3.81 (s, 1H), 3.64-3.52 (m, 1H), 3.50-3.36 (m, 2H), 1.75-1.32 (m, 6H), 0.92-0.74 (m, 1H), 0.70-0.42 (m, 3H).

Step 3. To a solution of commercially available ethyl 3-ethoxy-1H-pyrazole-5-carboxylate (1.20 g, 6.51 mmol, 1 eq) in THF (20 mL) was added (1-tetrahydropyran-2-yloxycyclopropyl)methanol (1.35 g, 7.82 mmol, 1.2 eq), DBAD (2.25 g, 9.77 mmol, 1.5 eq) and PPh₃ (3.42 g, 13.0 mmol, 2 eq) under N₂, then the mixture was stirred at 25° C. for 2 h. On completion, the reaction mixture was diluted with H₂O (50 mL) and extracted with EA (30 mL×3). The combined organic layers were washed with brine (20 mL×2), dried over Na₂SO₄, filtered, and concentrated under reduced pressure to give a residue. The residue was purified by flash silica gel chromatography to give ethyl 5-ethoxy-2-[(1-tetrahydropyran-2-yloxycyclopropyl)methyl]pyrazole-3-carboxylate (2.00 g, 91% yield) as colorless oil. ¹H NMR (400 MHz, DMSO-d₆) δ=6.26 (s, 1H), 4.72-4.65 (m, 1H), 4.64-4.60 (m, 1H), 4.57-4.50 (m, 1H), 4.32-4.24 (m, 2H), 4.17-4.06 (m, 2H), 3.77-3.61 (m, 1H), 3.40-3.32 (m, 1H), 1.66-1.55 (m, 1H), 1.52-1.34 (m, 5H), 1.31-1.27 (m, 6H), 1.14-1.04 (m, 1H), 0.76-0.65 (m, 2H), 0.64-0.55 (m, 1H). LCMS: (M+1-84:255.5).

Step 4. To a solution of the product from Step 3 (1.90 g, 5.61 mmol, 1 eq) in THF (20 mL) was added LiAlH₄ (2.5 M, 2.25 mL, 1 eq) at 0° C., and the mixture was stirred at 0° C. for 2 hr. On completion, the reaction mixture was quenched by addition sat. NH₄Cl (20 mL) at 0° C., and then diluted with H₂O (20 mL) and extracted with EA (15 mL×3). The combined organic layers were washed with brine (30 mL×2), dried over Na₂SO₄, filtered, and concentrated under reduced pressure to give [5-ethoxy-2-[(1-tetrahydropyran-2-yloxycyclopropyl)methyl]pyrazol-3-yl]methanol (1.65 g, crude) as a colorless oil. LCMS: (M+23:319.1).

Step 5. To a solution of [5-ethoxy-2-[(1-tetrahydropyran-2-yloxycyclopropyl)methyl]pyrazol-3-yl]methanol (1.55 g, 5.23 mmol, 1 eq) in ACN (15 mL) was added NIS (1.18 g, 5.23 mmol, 1 eq) at 0° C., and the mixture was stirred at 25° C. for 1 h. On completion, the reaction mixture was quenched by sat. Na₂SO₃ (10 mL) at 25° C., and then diluted with H₂O (20 mL) and extracted with EA (10 mL×3). The combined organic layers were washed with brine (10 mL×2), dried over Na₂SO₄, filtered, and concentrated under reduced pressure to give a residue. The residue was purified by flash silica gel chromatography to give [5-ethoxy-4-iodo-2-[(1-tetrahydropyran-2-yloxycyclopropyl)methyl]pyrazol-3-yl]methanol (2.04 g, 92% yield) as colorless oil. ¹H NMR (400 MHz, DMSO-d₆) δ=5.28 (t, J=5.2 Hz, 1H), 4.62-4.58 (m, 1H), 4.55-4.47 (m, 1H), 4.46-4.36 (m, 2H), 4.19-4.10 (m, 3H), 3.80-3.70 (m, 1H), 3.42-3.37 (m, 1H), 1.70-1.60 (m, 1H), 1.53-1.35 (m, 4H), 1.30 (t, J=7.2 Hz, 3H), 1.04 (d, J=12.4 Hz, 1H), 0.82-0.67 (m, 3H). LCMS: (M+23:445.1).

Step 6. To a solution of [5-ethoxy-4-iodo-2-[(1-tetrahydropyran-2-yloxycyclopropyl) methyl]pyrazol-3-yl]methanol (800 mg, 1.89 mmol, 1 eq) in DCM (10 mL) was added DMP (1.21 g, 2.84 mmol, 1.5 eq) at 0° C., and the mixture was stirred at 25° C. for 1 h. On completion, the reaction mixture was quenched by sat. Na₂SO₃ (10 mL) at 25° C., and then diluted with H₂O (20 mL) and extracted with DCM (8 mL×3). The combined organic layers were washed with brine (10 mL×2), dried over Na₂SO₄, filtered, and concentrated under reduced pressure to give a residue. The residue was purified by flash silica gel chromatography to give 5-ethoxy-4-iodo-2-[(1-tetrahydropyran-2-yloxycyclopropyl)methyl]pyrazole-3-carbaldehyde (550 mg, 69% yield) as colorless oil. LCMS: (M+1:420.8).

Step 7. To a solution of 2-(5-bromo-1-tetrahydropyran-2-yl-pyrazolo[3,4-c]pyridin-3-yl)ethynyl-triisopropyl-silane, which was prepared according to the method described in Ex. 2, (4.40 g, 9.51 mmol, 1 eq) in dioxane (50 mL) was added commercially available 2,5-dimethylpyrazol-3-ol (1.28 g, 11.4 mmol, 1.2 eq), K₂CO₃ (3.94 g, 28.5 mmol, 3 eq) and [2-(2-aminophenyl)phenyl]-methylsulfonyloxy-palladium; ditert-butyl-[3,6-dimethoxy-2-(2,4,6-triisopropylphenyl)phenyl]phosphane (tBuBrettPhos Pd G3) (813 mg, 951 μmol, 0.1 eq) under N₂, then the mixture was stirred at 130° C. for 1 h. On completion, the reaction mixture was diluted with H₂O (100 mL) and extracted with EA (30 mL×3). The combined organic layers were washed with brine (60 mL×2), dried over Na₂SO₄, filtered, and concentrated under reduced pressure to give a residue. The residue was purified by flash silica gel chromatography to give 2,5-dimethyl-4-|1-tetrahydropyran-2-yl-3-(2-triisopropylsilylethynyl) pyrazolo[3,4-c]pyridin-5-yl]pyrazol-3-ol (3.30 g, 70% yield) as yellow oil. LCMS: (M+1:494.2).

Step 8. To a solution of the product from Step 7 (3.10 g, 6.28 mmol, 1 eq) in THE (35 mL) was added commercially available 2,2,2-trifluoro-N-[(2R)-2-hydroxypropyl]-N-methyl-acetamide (1.16 g, 6.28 mmol, 1 eq), DBAD (2.17 g, 9.42 mmol, 1.5 eq) and PPh₃ (3.29 g, 12.6 mmol, 2 eq) under N₂, then the mixture was stirred at 25° C. for 2 h. On completion, the reaction mixture was diluted with H₂O (60 mL) and extracted with EA (20 mL×3). The combined organic layers were washed with brine (40 mL×2), dried over Na₂SO₄, filtered and concentrated under reduced pressure to give N-[(2S)-2-[2,5-dimethyl-4-[1-tetrahydropyran-2-yl-3-(2-triisopropylsilylethynyl)pyrazolo[3,4-c]pyridin-5-yl]pyrazol-3-yl]oxypropyl]-2,2,2-trifluoro-N-methyl-acetamide (4.20 g, crude) was obtained as a yellow oil. LCMS: (M+1:661.3).

Step 9. To a solution of the product from Step 8 (4.00 g, 6.05 mmol, 1 eq) in THE (40 mL) and H₂O (20 mL) was added LiOH·H₂O (762 mg, 18.2 mmol, 3 eq), then the mixture was stirred at 25° C. for 1 h. On completion, the reaction mixture was diluted with H₂O (60 mL) and extracted with EA (20 mL×3). The combined organic layers were washed with brine (40 mL×2), dried over Na₂SO₄, filtered, and concentrated under reduced pressure to give a residue. The residue was purified by flash silica gel chromatography to give (2S)-2-[2,5-dimethyl-4-[1-tetrahydropyran-2-yl-3-(2-triisopropylsilylethynyl)pyrazolo[3,4-c]pyridin-5-yl]pyrazol-3-yl]oxy-N-methyl-propan-1-amine (3.30 g, 96% yield) as yellow oil. LCMS: (M+1:565.5).

Step 10. To a solution of the product from Step 9 (500 mg, 885 μmol, 1 eq) in MeOH (10 mL) was added the product from Step 6 (446 mg, 1.06 mmol, 1.2 eq) and AcOH (5.32 mg, 88.5 mol, 0.1 eq) at 25° C. After the mixture was stirred at 25° C. for 0.5 h, NaBH₃CN (139 mg, 2.21 mmol, 2.5 eq) was added, and the mixture was stirred at 25° C. for 1 h. On completion, the reaction mixture was concentrated under reduced pressure to give a residue. The residue was purified by flash silica gel chromatography to give (2S)-2-[2,5-dimethyl-4-[1-tetrahydropyran-2-yl-3-(2-triisopropylsilylethynyl)pyrazolo[3,4-c]pyridin-5-yl]pyrazol-3-yl]oxy-N-[[5-ethoxy-4-iodo-2-[(1-tetrahydropyran-2-yloxycyclopropyl) methyl]pyrazol-3-yl]methyl]-N-methyl-propan-1-amine (200 mg, 23% yield) as yellow oil. LCMS: (M+1:969.4).

Step 11. To a solution of the product from Step 10 (120 mg, 0.124 mmol, 1 eq) in DMSO (1 mL) was added CsF (132 mg, 0.867 mmol, 7 eq), and the mixture was stirred at 25° C. for 1 h. On completion, the reaction mixture was diluted with H₂O (3 mL) and extracted with EA (1 mL×3). The combined organic layers were washed with brine (2 mL×2), dried over Na₂SO₄, filtered, and concentrated under reduced pressure to give a residue. The residue was purified by flash silica gel chromatography to give (2S)-N-[[5-ethoxy-4-iodo-2-[(1-tetrahydropyran-2-yloxycyclopropyl) methyl]pyrazol-3-yl]methyl]-2-[4-(3-ethynyl-1-tetrahydropyran-2-yl-pyrazolo[3,4-c]pyridin-5-yl)-2,5-dimethyl-pyrazol-3-yl]oxy-N-methyl-propan-1-amine (60.0 mg, 59% yield) as yellow oil. LCMS: (M+23(Na):835.3).

Step 12. To a solution of the product form Step 11 (50.0 mg, 0.0615 mmol, 1 eq) in dioxane (1 mL) was added BPD (31.2 mg, 0.123 mmol, 2 eq), Cu₂O (4.40 mg, 0.0308 mmol, 0.5 eq) and PPh₃ (16.1 mg, 0.0615 mmol, 1 eq) under N₂, and the mixture was stirred at 70° C. for 2 h. On completion, the reaction mixture was diluted with H₂O (3 mL) and extracted with EA (1 mL×3). The combined organic layers were washed with brine (2 mL×2), dried over Na₂SO₄, filtered, and concentrated under reduced pressure to give a residue. The residue was purified by flash silica gel chromatography to give (2S)-2-[2,5-dimethyl-4-[1-tetrahydropyran-2-yl-3-[(E)-2-(4,4,5,5-tetramethyl-1,3,2-dioxaborolan-2-yl)vinyl]pyrazolo[3,4-c]pyridin-5-yl]pyrazol-3-yl]oxy-N-[[5-ethoxy-4-iodo-2-[(1-tetrahydropyran-2-yloxycyclopropyl)methyl]pyrazol-3-yl]methyl]-N-methyl-propan-1-amine (50.0 mg, 86% yield) as yellow oil. LCMS: (M+1:941.3).

Step 13. To a solution of the product form Step 12 (45.0 mg, 47.8 mol, 1 eq) in DMA (0.5 mL) was added Na₂CO₃ (1.5 M, 95.7 μL, 3 eq) and dicyclohexyl-[2-(2,4,6-triisopropylphenyl)phenyl]phosphane; methanesulfonate; [2-[2-(methylamino)phenyl]phenyl]palladium(1+) (XPhos Pd G4) (4.12 mg, 4.78 μmol, 0.1 eq) under N₂, and the mixture was stirred at 80° C. for 2 h. On completion, the reaction mixture was diluted with H₂O (3 mL) and extracted with EA (1 mL×3). The combined organic layers were washed with brine (2 mL×2), dried over Na₂SO₄, filtered and concentrated under reduced pressure to give (10S,17E)-16-ethoxy-6,8,10,12-tetramethyl-2-(oxan-2-yl)-14-({1-[(oxan-2-yl)oxy]cyclopropyl}methyl)-2,10,11,12,13,14-hexahydro-8H-5,3-(azenometheno)tripyrazolo[3,4-f:3′,4′-j:4″,3″-n][1,4]oxazacyclopentadecine (50.0 mg, crude) as a yellow oil. LCMS: (M+1:687.4).

Step 14. To a solution of the crude product from Step 13 (45.0 mg, 0.0655 mmol, 1 eq) in DCM (0.5 mL) was added TFA (12.1 mmol, 0.900 mL, 185 eq), and the mixture was stirred at 25° C. for 1 h. On completion, the reaction mixture was concentrated under reduced pressure to give a residue. The residue was purified by prep-HPLC (column: Phenomenex luna C18 150*25 mm*10 um; mobile phase: [water(FA)-ACN]; gradient:15%-45% B over 10 min) to afford Ex. 129 (0.91 mg) as yellow solid. ¹H NMR for Ex. 129 can be found in the below NMR table.

General Method E: using Ex. 71 as example.

### Preparation of (17E)-8,15,16-trimethyl-2,10,11,12,13,15-hexahydro-8H-5,3-(azenometheno)tripyrazolo[3,4-f:3′,4′-j:4″,3″-n][1,4]oxazacyclopentadecine (Ex. 71)

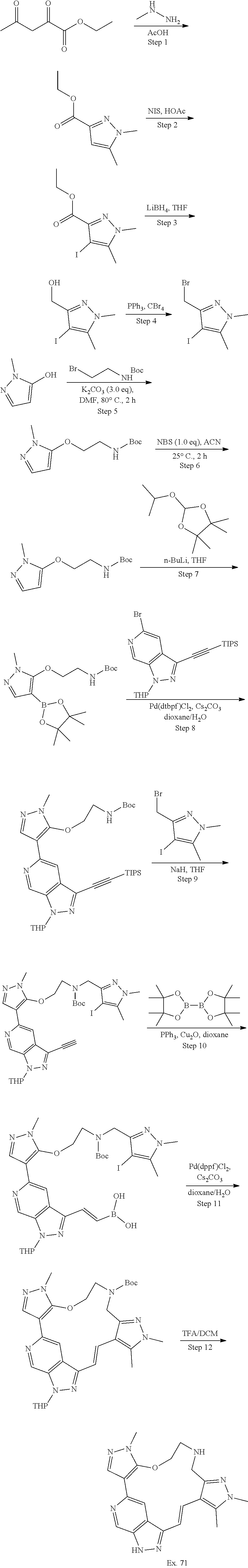

Step 1. To a mixture of commercially available ethyl 2,4-dioxopentanoate (20.0 g, 126 mmol, 17.8 mL, 1 eq) in AcOH (157 g, 2.62 mol, 150 mL, 20.7 eq) was added methylhydrazine (16.4 g, 142 mmol, 18.8 mL, 40% purity, 1.13 eq) at 0° C. The reaction mixture was stirred at 25° C. for 5 hours. On completion, the reaction mixture was concentrated in vacuo. The residue was diluted with water (200 mL) and extracted with EA(2×200 mL). The combined organic layer was dried over Na₂SO₄, filtered and concentrated in vacuo to give a residue. The residue was purified by column chromatography to afford ethyl 1,5-dimethyl-1H-pyrazole-3-carboxylate (17.4 g, 81% yield) as yellow solid. ¹H NMR (400 MHz, DMSO-d₆) δ6.51 (s, 1H), 4.22 (q, J=7.2 Hz, 2H), 3.78 (s, 3H), 2.26 (s, 3H), 1.26 (t, J=7.2 Hz, 3H).

Step 2. To a mixture of ethyl 1,5-dimethyl-1H-pyrazole-3-carboxylate (5.00 g, 29.7 mmol, 1 eq) in AcOH (50 mL) was added NIS (8.03 g, 35.6 mmol, 1.2 eq). The reaction mixture was stirred at 80° C. for 2 hours. On completion, the residue was diluted with water (80 mL) and extracted with EA(2×80 mL). The combined organic layer was dried over Na₂SO₄, filtered and concentrated in vacuo to give a residue. The residue was purified by column chromatography to afford ethyl 4-iodo-1,5-dimethyl-1H-pyrazole-3-carboxylate (8.00 g, 91% yield) as brown solid. ¹H NMR (400 MHz, DMSO-d₆) δ 4.25 (q, J=7.2 Hz, 2H), 3.89 (s, 3H), 2.31 (s, 3H), 1.29 (t, J=7.2 Hz, 3H). LCMS: (M+1:294.8).

Step 3. To a mixture of ethyl 4-iodo-1,5-dimethyl-1H-pyrazole-3-carboxylate (5.00 g, 17.0 mmol, 1 eq) in THE (50 mL) was added LiBH₄ (555 mg, 25.5 mmol, 1.5 eq) at 0° C. The reaction mixture was stirred at 25° C. for 12 hours. On completion, the reaction mixture was quenched with water (10 mL), the residue was diluted with water (60 mL) and extracted with EA(2×60 mL). The combined organic layers was dried over Na₂SO₄, filtered and concentrated in vacuo to afford (4-iodo-1,5-dimethyl-1H-pyrazol-3-yl)methanol (4.00 g, 93% yield) as white solid. ¹H NMR (400 MHz, DMSO-d₆) δ 4.87 (t, J=5.2 Hz, 1H), 4.27 (d, J=5.2 Hz, 2H), 3.76 (s, 3H), 2.23 (s, 3H). LCMS: (M+1:252.8).

Step 4. To a mixture of (4-iodo-1,5-dimethyl-1H-pyrazol-3-yl)methanol (3.50 g, 13.8 mmol, 1 eq) and PPh₃ (5.46 g, 20.8 mmol, 1.5 eq) in DCM (30 mL) was added CBr₄ (6.91 g, 20.8 mmol, 1.5 eq) at 0° C. The reaction mixture was stirred at 25° C. for 1 hour. On completion, the residue was diluted with water (50 mL) and extracted with EA(2×50 mL). The combined organic layer was dried over Na₂SO₄, filtered, and concentrated in vacuo to give a residue. The residue was purified by column chromatography to afford 3-(bromomethyl)-4-iodo-1,5-dimethyl-1H-pyrazole (3.50 g, 80% yield) as white solid. ¹H NMR (400 MHz, DMSO-d₆) δ 4.47 (s, 2H), 3.79 (s, 3H), 2.24 (s, 3H). LCMS: (M+1:316.5).

Step 5. To a solution of commercially available 1-methyl-1H-pyrazol-5-ol (20.0 g, 203 mmol, 1.0 eq) in DMF (400 mL) was added commercially available tert-butyl (2-bromoethyl)carbamate (54.8 g, 244 mmol, 1.2 eq) and K₂CO₃ (84.5 g, 611 mmol, 3.0 eq). The mixture was stirred at 80° C. for 2 h. On completion, the mixture was quenched with water (120 mL) and extracted with ethyl acetate (125 mL×3). The combined organic phase was dried over anhydrous sodium sulfate, filtered, and concentrated to give tert-butyl (2-((1-methyl-1H-pyrazol-5-yl)oxy)ethyl)carbamate (40.0 g, 81.32% yield) as a yellow oil. LCMS: (M+1: 242.1).

Step 6. To a solution of tert-butyl (2-((1-methyl-1H-pyrazol-5-yl)oxy)ethyl)carbamate (39.9 g, 165 mmol, 1.0 eq) in MeCN (400 mL) was added NBS (29.4 g, 165 mmol, 1.0 eq). The mixture was stirred at 25° C. for 2 h. On completion, the mixture was filtered and concentrated to give a residue. The residue was purified by column chromatography (SiO₂, Petroleum ether/Ethyl acetate=3:1 to 2:1) to give tert-butyl (2-((4-bromo-1-methyl-1H-pyrazol-5-yl)oxy)ethyl)carbamate (28.0 g, 52.8% yield) as a white oil. ¹H NMR (400 MHz, DMSO-d₆) S=7.38 (s, 1H), 7.12-7.02 (m, 1H), 4.21 (t, J=8.0 Hz, 2H), 3.63 (s, 3H), 3.30-3.26 (m, 2H), 1.39 (s, 9H).

Step 7. To a mixture of tert-butyl N-[2-(4-bromo-2-methyl-1H-pyrazol-3-yl)oxyethyl]carbamate (7.00 g, 21.9 mmol, 1 eq) in THE (60 mL) was added a solution of n-BuLi (2.5 M, 26.2 mL, 3 eq) in hexane dropwise at −70° C. The mixture was stirred at −70° C. for 1 hour. Then to the mixture was added 2-isopropoxy-4,4,5,5-tetramethyl-1,3,2-dioxaborolane (8.14 g, 43.7 mmol, 8.92 mL, 2 eq) dropwise at −70° C. The mixture was stirred at −70° C. for 0.5 hour under N₂. To the mixture was added water (3 mL). The mixture was concentrated in vacuum to give tert-butyl N-[2-[2-methyl-4-(4,4,5,5-tetramethyl-1,3,2-dioxaborolan-2-yl)-1H-pyrazol-3-yl]oxyethyl]carbamate (8.00 g, crude) as yellow solid. Spectra: LCMS: (M+1:367.8).

Step 8. To a mixture of 2-(5-bromo-1-tetrahydropyran-2-yl-pyrazolo[3,4-c]pyridin-3-yl)ethynyl-triisopropyl-silane, which was prepared according to the method described in Ex. 2 (2.80 g, 6.05 mmol, 1 eq), and tert-butyl N-[2-[2-methyl-4-(4,4,5,5-tetramethyl-1,3,2-dioxaborolan-2-yl)-1H-pyrazol-3-yl]oxyethyl]carbamate (4.45 g, 12.1 mmol, 2 eq) in dioxane (25 mL) and H₂O (5 mL) was added Cs₂CO₃ (5.92 g, 18.1 mmol, 3 eq) and ditert-butyl(cyclopentyl)phosphane; dichloropalladium; iron (Pd(dtbpf)Cl₂) (394 mg, 605 umol, 0.1 eq). The reaction mixture was stirred at 90° C. for 12 hours. On completion, the residue was diluted with water (40 mL) and extracted with EA (2×40 mL). The combined organic layers was dried over Na₂SO₄, filtered and concentrated in vacuo to give a residue. The residue was purified by column chromatography to afford tert-butyl N-[2-[2-methyl-4-[1-tetrahydropyran-2-yl-3-(2-triisopropylsilylethynyl)-1H-pyrazolo[3,4-c]pyridin-5-yl]pyrazol-3-yl]oxyethyl]carbamate (2.10 g, 55% yield) as brown oil. ¹H NMR (400 MHz, CDCl₃) δ 9.21 (s, 1H), 7.86-7.71 (m, 2H), 6.43 (s, 1H), 5.81 (dd, J=2.0, 8.8 Hz, 1H), 4.23 (t, J=4.8 Hz, 2H), 4.06-3.98 (m, 1H), 3.80 (s, 4H), 3.57-3.48 (m, 2H), 2.59-2.44 (m, 1H), 2.19-2.09 (m, 2H), 1.84-1.60 (m, 5H), 1.50 (s, 9H), 1.48-1.43 (m, 1H), 1.22-1.18 (m, 18H). LCMS: (M+1:624.1).

Step 9. To a mixture of tert-butyl N-[2-[2-methyl-4-[1-tetrahydropyran-2-yl-3-(2-triisopropylsilylethynyl)-1H-pyrazolo[3,4-c]pyridin-5-yl]pyrazol-3-yl]oxyethyl]carbamate (276 mg, 444 umol, 0.5 eq) in THE (5 mL) was added NaH (53.3 mg, 60% purity, 1.5 eq) at 0° C. The reaction mixture was stirred at 0° C. for 0.5 hour. Then the reaction was added 3-(bromomethyl)-4-iodo-1,5-dimethyl-pyrazole (280 mg, 0.889 mmol, 1 eq). The reaction mixture was stirred at 25° C. for 12 hours. On completion, the reaction mixture was quenched with water (1 mL). The residue was diluted with water (20 mL) and extracted with EA(2×20 mL). The combined organic layers was dried over Na₂SO₄, filtered and concentrated in vacuo to give a residue. The residue was purified by reverse phase column chromatograph (0.1% FA in mobile phase) to afford tert-butyl N-[2-[4-(3-ethynyl-1-tetrahydropyran-2-yl-pyrazolo[3,4-c]pyridin-5-yl)-2-methyl-pyrazol-3-yl]oxyethyl]-N-[(4-iodo-1,5-dimethyl-pyrazol-3-yl)methyl]carbamate (140 mg, 22% yield) as yellow oil. ¹H NMR (400 MHz, DMSO-d₆) δ 9.27 (s, 1H), 7.90 (s, 1H), 7.79 (s, 1H), 6.02 (dd, J=2.0, 9.20 Hz, 1H), 4.68 (s, 1H), 4.44-4.35 (m, 2H), 4.22-4.10 (m, 2H), 3.88 (s, 1H), 3.82-3.75 (m, 1H), 3.67 (s, 6H), 3.57-3.45 (m, 2H), 2.36-2.36-2.29 (m, 1H), 2.18 (s, 3H), 2.08-2.00 (m, 2H), 1.84-1.70 (m, 1H), 1.65-1.57 (m, 2H), 1.38-1.28 (m, 9H). LCMS: (M+1:701.2).

Step 10. To a mixture of tert-butyl N-[2-[4-(3-ethynyl-1-tetrahydropyran-2-yl-pyrazolo[3,4-c]pyridin-5-yl)-2-methyl-pyrazol-3-yl]oxyethyl]-N-[(4-iodo-1,5-dimethyl-pyrazol-3-yl)methyl]carbamate (60.0 mg, 85.6 umol, 1 eq) and commercially available 4,4,4′,4′,5,5,5′,5′-octamethyl-2,2′-bi(1,3,2-dioxaborolane (21.7 mg, 85.6 umol, 1 eq) in dioxane (2 mL) was added Cu₂O (1.23 mg, 8.56 umol, 0.1 eq) and PPh₃ (22.4 mg, 85.6 umol, 1 eq). The reaction mixture was stirred at 60° C. for 12 hours. On completion, the residue was diluted with water (10 mL) and extracted with EA(2×10 mL). The combined organic layer was dried over Na₂SO₄, filtered and concentrated in vacuo to give a residue. The residue was purified by prep-HPLC (column: Welch Xtimate C18 150*25 mm*5 um; mobile phase: [water(TFA)-ACN]; B %: 51%-81%,10 min) to afford [(E)-2-[5-[5-[2-[tert-butoxycarbonyl-[(4-iodo-1,5-dimethyl-pyrazol-3-yl)methyl]amino]ethoxy]-1-methyl-pyrazol-4-yl]-1-tetrahydropyran-2-yl-pyrazolo[3,4-c]pyridin-3-yl]vinyl]boronic acid (30.0 mg, 46% yield) as yellow solid. ¹H NMR (400 MHz, DMSO-d₆) δ 9.22 (s, 1H), 8.16-8.02 (m, 1H), 7.98-7.83 (m, 1H), 7.54 (d, J=18.8 Hz, 1H), 7.48-7.37 (m, 2H), 7.34-7.26 (m, 1H), 6.61-6.40 (m, 1H), 6.05-5.93 (m, 1H), 4.45-4.30 (m, 2H), 4.24-4.13 (m, 2H), 3.67-3.65 (m, 2H), 3.56-3.43 (m, 3H), 2.16 (s, 3H), 2.04 (d, J=10.0 Hz, 2H), 1.84-1.72 (m, 1H), 1.69-1.58 (m, 2H), 1.40-1.28 (m, 9H), 1.26 (s, 5H). LCMS: (M+1:747.0).

Step 11. To a mixture of [(E)-2-[5-[5-[2-[tert-butoxycarbonyl-[(4-iodo-1,5-dimethyl-pyrazol-3-yl)methyl]amino]ethoxy]-1-methyl-pyrazol-4-yl]-1-tetrahydropyran-2-yl-pyrazolo [3,4-c]pyridin-3-yl]vinyl]boronic acid (20.0 mg, 26.80 umol, 1 eq) in dioxane (1 mL) and H₂O (0.2 mL) was added Pd(dppf)Cl₂ (1.96 mg, 2.68 umol, 0.1 eq) and Cs₂CO₃ (26.1 mg, 80.3 umol, 3 eq). The reaction mixture was stirred at 90° C. for 12 hours. On completion, the residue was diluted with water (10 mL) and extracted with EA(2×10 mL). The combined organic phase was dried over Na₂SO₄, filtered and concentrated in vacuo to give a residue. the residue was purified by column chromatography to afford tert-butyl (17E)-8,15,16-trimethyl-2-(oxan-2-yl)-2,8,10,11,13,15-hexahydro-12H-5,3-(azenometheno)tripyrazolo[3,4-f:3′,4′-j:4″,3″-n][1,4]oxazacyclopentadecine-12-carboxylate (10.0 mg, 64% yield) as yellow solid. LCMS: (M+1:575.3).

Step 12. To a mixture of tert-butyl (17E)-8,15,16-trimethyl-2-(oxan-2-yl)-2,8,10,11,13,15-hexahydro-12H-5,3-(azenometheno)tripyrazolo[3,4-f:3′,4′-j:4″,3″-n][1,4]oxazacyclopentadecine-12-carboxylate (9.00 mg, 15.6 umol, 1 eq) in DCM (0.5 mL) was added TFA (6.93 g, 60.7 mmol, 4.50 mL, 388 eq). The reaction mixture was stirred at 25° C. for 1 hour. On completion, the residue was purified by prep-HPLC (column: Welch Ultimate C18 150*25 mm*5 um; mobile phase: [water(TFA)-ACN]; B %: 2%-32%, 2 min) to afford Ex. 71 (1.16 mg, 14% yield, 96% purity, TFA) as yellow solid. LCMS: (M+1:391.2). ¹H NMR for Ex. 71 is shown in the below NMR table.

### Preparation of (17E)-8,12,15,16-tetramethyl-2,10,11,12,13,15-hexahydro-8H-5,3-(azenometheno)tripyrazolo[3,4-f:3′,4′-j:4″,3″-n][1,4]oxazacyclopentadecine (Ex. 73)

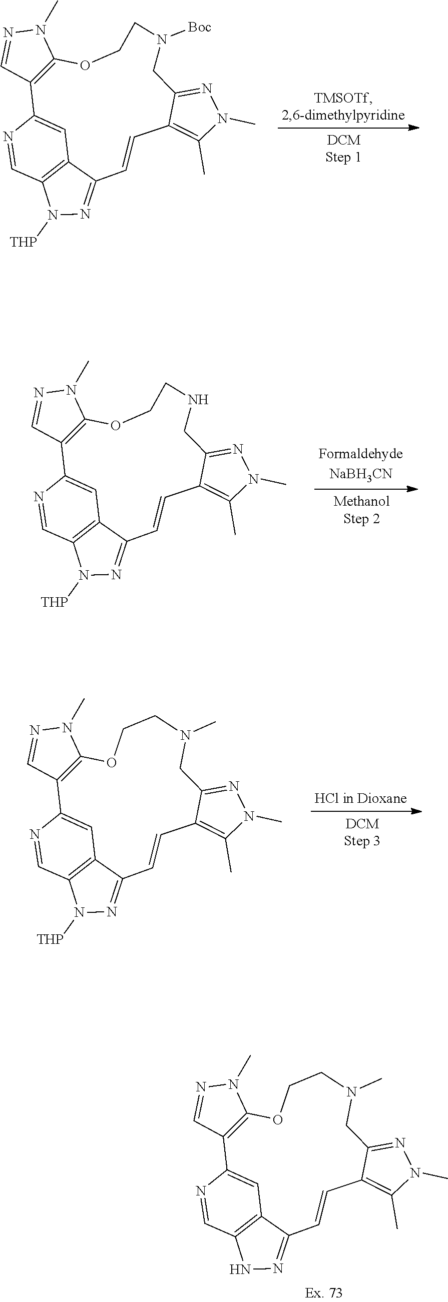

Step 1. To a mixture of tert-butyl (17E)-8,15,16-trimethyl-2-(oxan-2-yl)-2,8,10,11,13,15-hexahydro-12H-5,3-(azenometheno)tripyrazolo[3,4-f:3′,4′-j:4″,3″-n][1,4]oxazacyclopentadecine-12-carboxylate, which was prepared according to the method described in Ex. 71, (45.0 mg, 78.3 umol, 1 eq) in DCM (1 mL) was added TMSOTf (26.1 mg, 117 umol, 21.2 uL, 1.5 eq) and 2,6-dimethylpyridine (16.9 mg, 156 umol, 18.4 uL, 2 eq). The reaction mixture was stirred at 25° C. for 1 hour. On completion, the mixture was added to sat. NaHCO₃ (10 mL) to quench the reaction, extracted with DCM (20 mL×2), washed with brine (10 mL), dried over Na₂SO₄, filtered and concentrated in vacuum to give the residue. The residue was purified by prep-HPLC (column: Phenomenex C18 150*25 mm*10 um; mobile phase: [water(NH₄HCO₃)-ACN]; B %: 15%-45%, 8 min) to afford (17E)-8,15,16-trimethyl-2-(oxan-2-yl)-2,10,11,12,13,15-hexahydro-8H-5,3-(azenometheno)tripyrazolo[3,4-f:3′,4′-j:4″,3″-n][1,4]oxazacyclopentadecine (4.51 mg, 8.86 umol, 11% yield, 93% purity) as yellow solid. ¹H NMR (400 MHz, DMSO-cd₆) δ 9.25 (s, 1H), 8.58 (s, 1H), 8.36 (d, J=17.6 Hz, 1H), 7.99-7.93 (m, 1H), 7.53 (d, J=14.0 Hz, 1H), 6.89 (d, J=17.6 Hz, 1H), 6.29-6.19 (m, 1H), 4.48 (t, J=5.2 Hz, 1H), 4.19 (s, 2H), 3.86 (d, J=7.6 Hz, 2H), 3.74 (s, 6H), 3.70 (d, J=3.6 Hz, 2H), 3.50 (q, J=6.2 Hz, 2H), 3.10 (d, J=2.8 Hz, 2H), 2.36-2.27 (m, 4H), 1.68-1.60 (m, 2H). LCMS: (M+1:475.1).

Step 2. To a solution of (17E)-8,15,16-trimethyl-2-(oxan-2-yl)-2,10,11,12,13,15-hexahydro-8H-5,3-(azenometheno)tripyrazolo[3,4-f:3′,4′-j:4″,3″-n][1,4]oxazacyclopentadecine (3.32 mg, 7.00 μmol) in Methanol (0.1 mL) was added Formaldehyde, 37% in aq. soln. (336.14 g, 11.20 mol, 0.31 μL), followed by Sodium cyanoborohydride (1.0 mg, 15.91 μmol). The mixture was stirred at 23° C. for 3 hr. The reaction mixture was then diluted with water (0.1 mL) and extracted with ethyl acetate (3×1 mL), washed with brine, and dried over sodium sulfate. The volatiles were evaporated in vacuo and the crude residue was taken over to the next step without further purification.

Step 3. To a solution of (17E)-8,12,15,16-tetramethyl-2-(oxan-2-yl)-2,10,11,12,13,15-hexahydro-8H-5,3-(azenometheno)tripyrazolo[3,4-f:3′,4′-j:4″,3″-n∥1,4]oxazacyclopentadecine (crude from Step 2, 3.42 mg, 7.00 μmol) in Dichloromethane (0.6 mL) was added HCl in Dioxane (4.0 M, 0.1 mL) and stirred for 24 h. The volatiles were evaporated in vacuo and the residue was purified using preparative HPLC (C18, 21 mm×100 mm, Acetonitrile in water with 0.035% TFA) to afford Ex. 73 (1.01 mg, 1.95 μmol, 27.8% yield, TFA salt). LCMS: (M+1: 404.8). ¹H NMR for Ex. 73 is shown in the below NMR table.

General Method F: using Ex. 132 as an example.

### Preparation of 2-[(10S,17E)-8,10,12-trimethyl-16-[(propan-2-yl)oxy]-2,8,10,11,12,13-hexahydro-14H-5,3-(azenometheno)tripyrazolo[3,4-f:3,4′-j:4″,3″-n][1,4]oxazacyclopentadecin-14-yl]ethan-1-ol (Ex. 132)

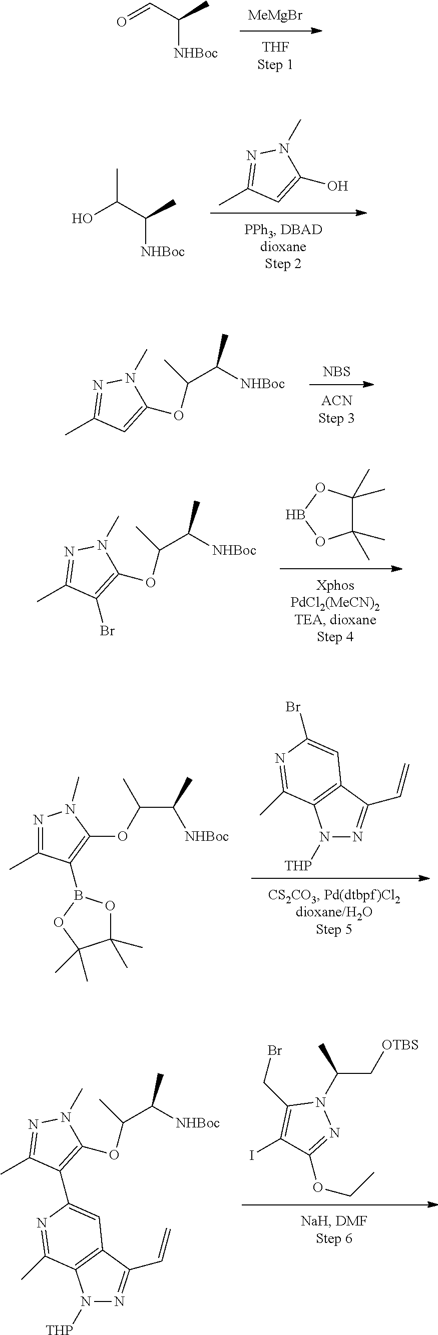

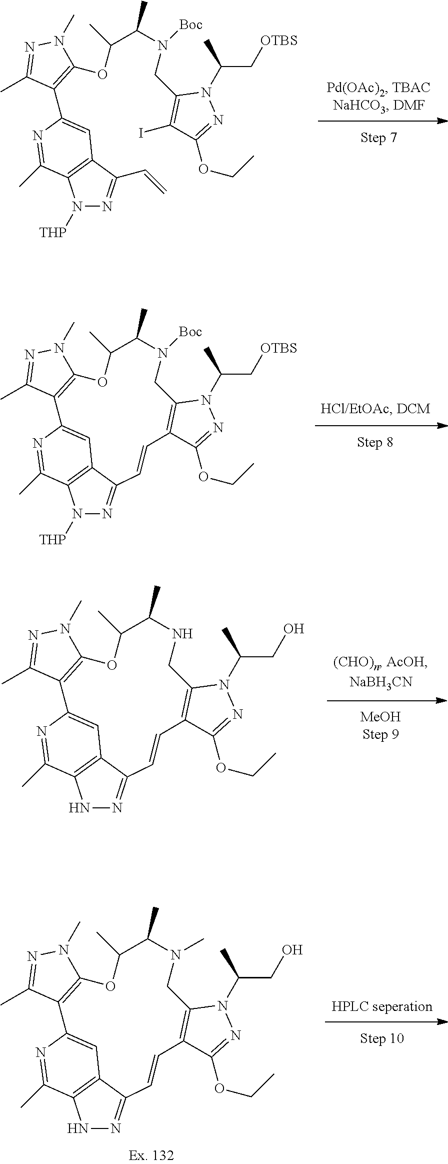

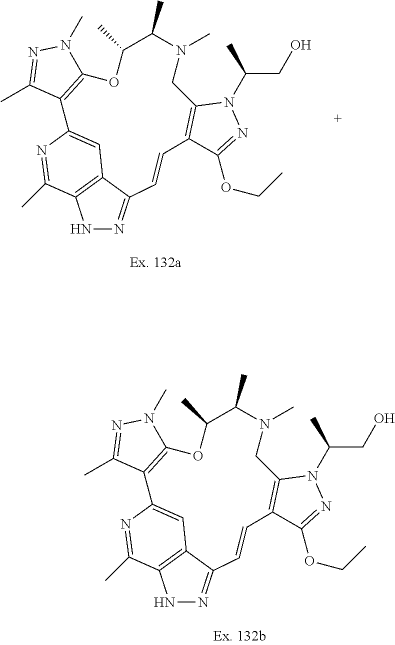 [SMILES: CCOc1nn([C@@H](C)CO)c2c1/C=C/c1n[nH]c3c(C)nc(cc13)-c1c(C)nn(C)c1O[C@H](C)[C@@H](C)N(C)C2 ; CCOc1nn([C@@H](C)CO)c2c1/C=C/c1n[nH]c3c(C)nc(cc13)-c1c(C)nn(C)c1O[C@@H](C)[C@@H](C)N(C)C2]

Step 1. To a solution of tert-butyl (R)-(1-oxopropan-2-yl)carbamate (5.00 g, 28.8 mmol, 1 eq) in THE (50 mL) was added bromo(methyl)magnesium (3 M, 19 mL, 2 eq). The mixture was stirred at 0° C. for 0.5 hours. On completion, the mixture was quenched with water (5 mL) and extracted with ethyl acetate (5 mL×3). The combined organic phase was dried over anhydrous sodium sulfate, filtered, and concentrated to give a residue. The residue was purified by column chromatography (SiO₂, Petroleum ether/Ethyl acetate=1:0 to 2:1) to give tert-butyl ((2R)-3-hydroxybutan-2-yl)carbamate (4.10 g, 21.6 mmol, 75% yield) as colorless oil. ¹H NMR (400 MHz, CDCl₃) δ=4.69 (s, 1H), 3.74-3.63 (m, 1H), 3.56 (d, J=5.6 Hz, 1H), 1.45 (s, 9H), 1.19 (d, J=6.4 Hz, 3H), 1.16 (d, J=6.8 Hz, 3H).

Step 2. A mixture of tert-butyl ((2R)-3-hydroxybutan-2-yl)carbamate (2.00 g, 10.5 mmol, 1 eq), 1,3-dimethyl-1H-pyrazol-5-ol (1.07 g, 9.51 mmol, 0.9 eq), PPh₃ (5.54 g, 21.1 mmol, 2 eq) in dioxane (30 mL) was degassed and purged with N₂ for 3 times. DBAD (3.65 g, 15.8 mmol, 1.5 eq) was added at 0° C., and then the mixture was stirred at 25° C. for 0.5 hours under N₂ atmosphere. On completion, the mixture was concentrated to give a residue. The residue was purified by column chromatography (SiO₂, Petroleum ether/THF=1:0 to 2:1) to give tert-butyl ((2R)-3-((1,3-dimethyl-1H-pyrazol-5-yl)oxy)butan-2-yl)carbamate (1.41 g, 4.98 mmol, 47% yield) as colorless oil. LCMS: (M+1: 284.0).

Step 3. To a solution of tert-butyl ((2R)-3-((1,3-dimethyl-1H-pyrazol-5-yl)oxy)butan-2-yl)carbamate (1.31 g, 4.62 mmol, 1 eq) in ACN (15 mL) was added NBS (987 mg, 5.55 mmol, 1.2 eq). The mixture was stirred at 0° C. for 10 minutes. On completion, the mixture was quenched with the saturated solution of Na₂SO₃ (15 mL) and extracted with ethyl acetate (10 mL×3). The combined organic phase was dried over anhydrous sodium sulfate, filtered, and concentrated to give a residue. The residue was purified by column chromatography (SiO₂, Petroleum ether/Ethyl acetate=1:0 to 2:1) to give tert-butyl ((2R)-3-((4-bromo-1,3-dimethyl-1H-pyrazol-5-yl)oxy)butan-2-yl)carbamate (1.27 g, 3.51 mmol, 75% yield) as colorless oil. LCMS: (M+23: 383.9).

Step 4. A mixture of tert-butyl ((2R)-3-((4-bromo-1,3-dimethyl-1H-pyrazol-5-yl)oxy)butan-2-yl)carbamate (1.17 g, 3.23 mmol, 1 eq), 4,4,5,5-tetramethyl-1,3,2-dioxaborolane (1.24 g, 9.69 mmol, 3 eq), dicyclohexyl-[2-(2,4,6-triisopropylphenyl) phenyl]phosphane (153 mg, 0.322 mmol, 0.1 eq), bis(acetonitrile)dichloropalladium(II) (83.7 mg, 0.322 mmol, 0.1 eq) and TEA (980 mg, 9.69 mmol, 1.4 mL, 3 eq) in dioxane (10 mL) was degassed and purged with N₂ for 3 times, and then the mixture was stirred at 110° C. for 2 hours under N₂ atmosphere. On completion, the mixture was filtered and concentrated to give a residue. The residue was purified by column chromatography (SiO₂, Petroleum ether/Ethyl acetate=1:0 to 2:1) to give tert-butyl ((2R)-3-((1,3-dimethyl-4-(4,4,5,5-tetramethyl-1,3,2-dioxaborolan-2-yl)-1H-pyrazol-5-yl)oxy)butan-2-yl)carbamate (1.05 g, 2.57 mmol, 79% yield) as colorless oil. LCMS: (M+1: 410.2).

Step 5. A mixture of tert-butyl ((2R)-3-((1,3-dimethyl-4-(4,4,5,5-tetramethyl-1,3,2-dioxaborolan-2-yl)-1H-pyrazol-5-yl)oxy)butan-2-yl)carbamate 952 mg, 2.33 mmol, 1.5 eq), 5-bromo-7-methyl-1-(tetrahydro-2H-pyran-2-yl)-3-vinyl-1H-pyrazolo[3,4-c]pyridine (500 mg, 1.55 mmol, 1 eq, from Ex. 28), [1,1′-Bis(di-tert-butylphosphino)ferrocene]dichloropalladium(II) (101 mg, 0.155 mmol, 0.1 eq), Cs₂CO₃ (1.52 g, 4.66 mmol, 3 eq) in dioxane (10 mL) and H₂O (2 mL) was degassed and purged with N₂ thrice, and then the mixture was stirred at 80° C. for 1 hour under N₂ atmosphere. On completion, the mixture was filtered and concentrated to give a residue. The residue was purified by column chromatography (SiO₂, Petroleum ether/Ethyl acetate=1:0 to 0:1) to give tert-butyl ((2R)-3-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)butan-2-yl)carbamate (800 mg, 1.52 mmol, 98% yield) as green oil. LCMS: (M+1: 525.2).

Step 6. To a solution of tert-butyl ((2R)-3-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)butan-2-yl)carbamate (300 mg, 0.571 mmol, 1 eq) in DMF (5 mL) was added NaH (34.3 mg, 0.857 mmol, 60% purity, 1.5 eq) at 0° C., followed by (S)-5-(bromomethyl)-1-(1-((tert-butyldimethylsilyl)oxy)propan-2-yl)-3-ethoxy-4-iodo-1H-pyrazole (287 mg, 0.571 mmol, 1 eq, from Ex. 19). The mixture was stirred at 25° C. for 0.5 hours. On completion, the mixture was quenched with water (15 mL) and extracted with ethyl acetate (10 mL×3). The combined organic phase was dried over anhydrous sodium sulfate, filtered, and concentrated to give a residue. The residue was purified by column chromatography (SiO₂, Petroleum ether/Ethyl acetate=1:0 to 1:2) to give tert-butyl ((1-((S)-1-((tert-butyldimethylsilyl)oxy)propan-2-yl)-3-ethoxy-4-iodo-1H-pyrazol-5-yl)methyl)((2R)-3-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)butan-2-yl)carbamate (200 mg, 0.211 mmol, 36% yield) as a yellow solid. LCMS: (M+1: 947.3)

Step 7. A mixture of tert-butyl ((1-((S)-1-((tert-butyldimethylsilyl)oxy)propan-2-yl)-3-ethoxy-4-iodo-1H-pyrazol-5-yl)methyl)((2R)-3-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)butan-2-yl)carbamate (100 mg, 0.105 mmol, 1 eq), Pd(OAc)₂ (4.74 mg, 0.021 mmol, 0.2 eq), TBAC (29.3 mg, 0.105 mmol, 1 eq) and NaHCO₃ (22.1 mg, 0.263 mmol, 2.5 eq) in DMF (1 mL) was degassed and purged with N₂ for 3 times, and then the mixture was stirred at 120° C. for 0.5 hours under N₂ atmosphere. On completion, the mixture was quenched with water (8 mL) and extracted with ethyl acetate (5 mL×3). The combined organic phase was dried over anhydrous sodium sulfate, filtered, and concentrated to give a residue. The residue was purified by column chromatography (SiO₂, Petroleum ether/Ethyl acetate=1:0 to 1:1) to give tert-butyl (11R,17E)-14-[(2S)-1-{[tert-butyl(dimethyl)silyl]oxy}propan-2-yl]-16-ethoxy-6,8,10,11,20-pentamethyl-2-(oxan-2-yl)-2,8,10,11,13,14-hexahydro-12H-5,3-(azenometheno)tripyrazolo[3,4-f:3′,4′-j:4″,3″-n][1,4]oxazacyclopentadecine-12-carboxylate (65.0 mg, 0.079 mmol, 75% yield) as a yellow solid. LCMS: (M+1: 819.4).

Step 8. To a solution of tert-butyl (11R,17E)-14-[(2S)-1-{[tert-butyl(dimethyl)silyl]oxy}propan-2-yl]-16-ethoxy-6,8,10,11,20-pentamethyl-2-(oxan-2-yl)-2,8,10,11,13,14-hexahydro-12H-5,3-(azenometheno)tripyrazolo[3,4-f:3′,4′-j:4″,3″-n][1,4]oxazacyclopentadecine-12-carboxylate (65.0 mg, 0.079 mmol, 1 eq) in DCM (0.5 mL) was added HCl/EtOAc (2 M, 0.4 mL, 10 eq). The mixture was stirred at 25° C. for 1 hour. On completion, the mixture was concentrated to give (2S)-2-[(11R,17E)-16-ethoxy-6,8,10,11,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (40.0 mg, 0.072 mmol, 90% yield, HCl) as a yellow solid. LCMS: (M+1: 521.1).

Step 9. To a solution of (2S)-2-[(11R,17E)-16-ethoxy-6,8,10,11,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (40.0 mg, 0.072 mmol, 1 eq, HCl) and Paraformaldehyde (40.0 mg) in MeOH (1 mL). After addition AcOH (4.31 mg, 0.072 mmol, 1 eq) was added to solution until pH 5-6 at 25° C. for 0.5 hours. And then NaBH₃CN (5.41 mg, 0.086 mmol, 1.2 eq) was added at 25° C. for 0.5 hours. The mixture was stirred at 25° C. for 1 hour. On completion, the mixture was filtered and concentrated to give a residue. The residue was purified by prep-HPLC to give (2S)-2-[(11R,17E)-16-ethoxy-6,8,10,11,12,20-hexamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (3.52 mg, 0.0065 mmol, 9.03% yield, 98.52% purity) as a white solid (Ex. 132a/b).

Step 10. Diastereomers 132a and 132b were separated by HPLC. LC/MS and ¹H NMR for 132a and 132b can be found in the below NMR table.

General Method G: Using Ex. 135 as an example.

### Preparation of 1-[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]propan-1-one (Ex. 135)

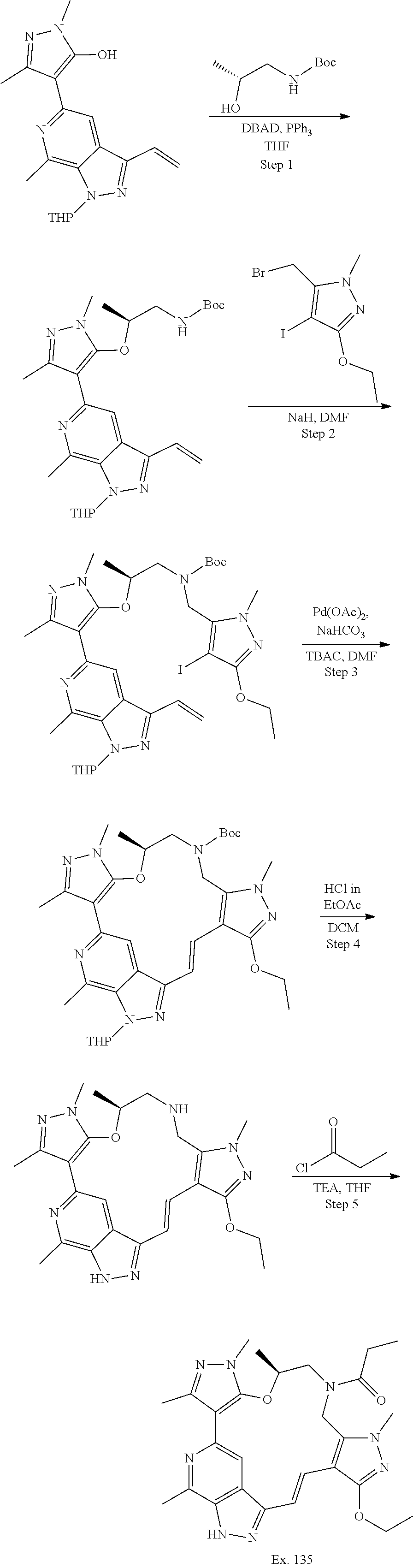

Step 1. A mixture of 1,3-dimethyl-4-(7-methyl-1-(tetrahydro-2H-pyran-2-yl)-3-vinyl-1H-pyrazolo[3,4-c]pyridin-5-yl)-1H-pyrazol-5-ol, which was prepared according to the method described in Ex. 47 (1.00 g, 2.83 mmol, 1 eq), tert-butyl (R)-(2-hydroxypropyl)carbamate (744 mg, 4.24 mmol, 1.5 eq), DBAD (1.43 g, 6.22 mmol, 2.2 eq), and PPh₃ (1.63 g, 6.22 mmol, 2.2 eq) in THE (10 mL) was degassed and purged with N₂, and then the mixture was stirred at 25° C. for 1 h under N₂ atmosphere. On completion, the reaction mixture was concentrated under reduced pressure to remove solvent. The residue was purified by column chromatography (SiO₂, Petroleum ether/THF=1:0 to 0:1) to give tert-butyl ((2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)propyl)carbamate (900 mg, 0.881 mmol, 31% yield, 50% purity) as a green oil. LCMS: (M+1=511.2).

Step 2. To a solution of tert-butyl ((2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)propyl)carbamate (850 mg, 0.832 mmol, 1 eq) in DMF (10 mL) was added NaH (83.2 mg, 2.08 mmol, 60% purity, 2.5 eq) at 0° C. The mixture was stirred at 0° C. for 0.5 h, and then was added 5-(bromomethyl)-3-ethoxy-4-iodo-1-methyl-1H-pyrazole, which was prepared according to the method described in Ex. 131 (287 mg, 0.832 mmol, 1 eq) at 0° C., and the mixture was stirred at 25° C. for 0.5 h. On completion, the mixture was added with saturated solution of NH₄Cl (20 mL), then partitioned between ethyl acetate (20 mL×3) and water (50 mL). The combined organic phase was washed with brine (100 mL), dried over anhydrous sodium sulfate, filtered, and concentrated to give a residue. The residue was purified by column chromatography (SiO₂, Petroleum ether/Ethyl acetate=1:0 to 0:1) to give tert-butyl ((2S)-2-((1,3-dimethyl-4-(7-methyl-1-(tetrahydro-2H-pyran-2-yl)-3-vinyl-1H-pyrazolo [3,4-c]pyridin-5-yl)-1H-pyrazol-5-yl)oxy)propyl)((3-ethoxy-4-iodo-1-methyl-1H-pyrazol-5-yl)methyl)carbamate (530 mg, 0.684 mmol, 82% yield) as yellow solid. LCMS: (M+1=775.5)

Step 3. A mixture of tert-butyl ((2S)-2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)propyl)((3-ethoxy-4-iodo-1-methyl-1H-pyrazol-5-yl)methyl)carbamate (480 mg, 0.620 mmol, 1 eq), TBAC (172 mg, 0.620 mmol, 1 eq), NaHCO₃ (130 mg, 1.55 mmol, 2.5 eq) and Pd(OAc)₂ (27.8 mg, 0.124 mmol, 0.2 eq) in DMF (5 mL) was degassed and purged with N₂ thrice, and then the mixture was stirred at 130° C. for 2 h under N₂ atmosphere. On completion, the mixture was quenched with water (50 mL) and extracted with ethyl acetate (25 mL×3). The combined organic phase was dried over anhydrous sodium sulfate, filtered, and concentrated to give a residue. The residue was purified by column chromatography (SiO₂, Petroleum ether/THF=1:0 to 0:1) to give tert-butyl (10S,17E)-16-ethoxy-6,8,10,14,20-pentamethyl-2-(oxan-2-yl)-10,11,13,14-tetrahydro-8H-5,3-(azenometheno)tripyrazolo[3,4-f:3′,4′-j:4″,3″-n][1,4]oxazacyclopentadecine-12(2H)-carboxylate (280 mg, 0.433 mmol, 70% yield) as yellow solid. LCMS: (M+1=647.4).

Step 4. To a solution of tert-butyl (10S,17E)-16-ethoxy-6,8,10,14,20-pentamethyl-2-(oxan-2-yl)-10,11,13,14-tetrahydro-8H-5,3-(azenometheno)tripyrazolo[3,4-f:3′,4′-j:4″,3″-n][1,4]oxazacyclopentadecine-12(2H)-carboxylate (260 mg, 0.402 mmol, 1 eq) in DCM (2 mL) was added HCl/EtOAc (2 M, 0.201 mL, 1 eq). The mixture was stirred at 25° C. for 1 h. On completion, the reaction mixture was concentrated under reduced pressure to remove solvent and give (10S,17E)-16-ethoxy-6,8,10,14,20-pentamethyl-2,10,11,12,13,14-hexahydro-8H-5,3-(azenometheno)tripyrazolo[3,4-f:3′,4′-j:4″,3″-n][1,4]oxazacyclopentadecine (200 mg, 0.401 mmol, 99% yield, HCl) as a yellow solid. LCMS: (M+1=463.3).

Step 5. To a solution of (10S,17E)-16-ethoxy-6,8,10,14,20-pentamethyl-2,10,11,12,13,14-hexahydro-8H-5,3-(azenometheno)tripyrazolo[3,4-f:3′,4′-j:4″,3″-n][1,4]oxazacyclopentadecine (100 mg, 0.216 mmol, 1 eq) in THE (1 mL) was added TEA (65.6 mg, 0.648 mmol, 3 eq) and propanoyl chloride (20.0 mg, 0.216 mmol, 1 eq). The mixture was stirred at 25° C. for 1 h. On completion, the mixture was quenched with water (5 mL) and extracted with ethyl acetate (5 mL×3), and the combined organic phase was dried over anhydrous sodium sulfate, filtered, and concentrated to give a residue. The residue was purified by prep-HPLC to give 1-[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]propan-1-one (6.07 mg, 0.011 mmol, 5.00% yield, 92.28% purity) as an orange solid (Ex. 135). ¹H NMR for Ex. 135 can be found in the below NMR table.

### Preparation of cyclopropyl[(10S,17E)-16-ethoxy-6,8,10,14,20-pentamethyl-2,8,10,11,13,14-hexahydro-12H-5,3-(azenometheno)tripyrazolo[3,4-f:3,4′-j:4″,3″-n][1,4]oxazacyclopentadecin-12-yl]methanone (Ex. 136)

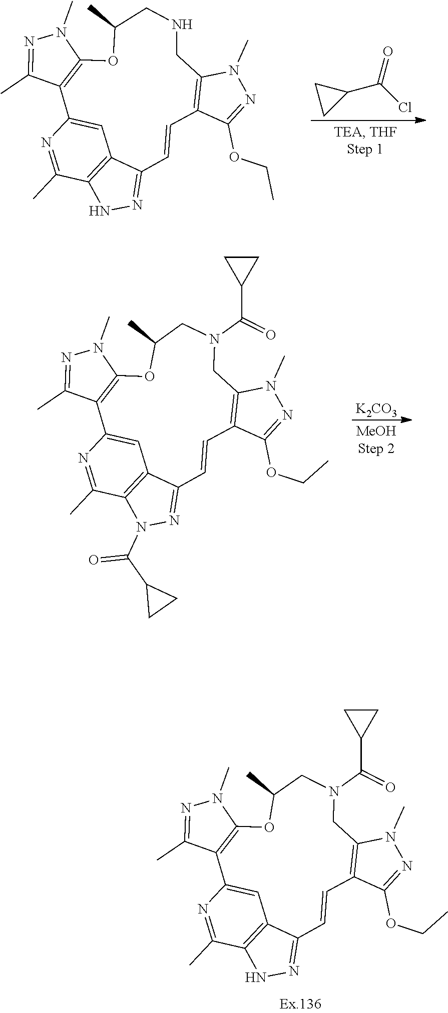

Step 1. To a solution of (10S,17E)-16-ethoxy-6,8,10,14,20-pentamethyl-2,10,11,12,13,14-hexahydro-8H-5,3-(azenometheno)tripyrazolo[3,4-f:3′,4′-j:4″,3″-n][1,4]oxazacyclopentadecine, which was prepared according to the method described in Ex. 135 (30.0 mg, 0.065 mmol, 1 eq) in THE (1 mL) was added TEA (19.7 mg, 0.195 mmol, 0.027 mL, 3 eq) and cyclopropanecarbonyl chloride (6.78 mg, 0.065 mmol, 0.0059 mL, 1 eq). The mixture was stirred at 0° C. for 1 hr. On completion, the mixture was quenched with water (10 mL) and extracted with ethyl acetate (10 mL×3), and the combined organic phase was dried over anhydrous sodium sulfate, filtered and concentrated to give [(10S,17E)-16-ethoxy-6,8,10,14,20-pentamethyl-10,11,13,14-tetrahydro-8H-5,3-(azenometheno)tripyrazolo[3,4-f:3′,4′-j:4″,3″-n][1,4]oxazacyclopentadecine-2,12-diyl]bis(cyclopropylmethanone) (30.0 mg, 0.050 mmol, 77% yield) as a colorless oil. LCMS: (M+1=599.3).

Step 2. To a solution of [(10S,17E)-16-ethoxy-6,8,10,14,20-pentamethyl-10,11,13,14-tetrahydro-8H-5,3-(azenometheno)tripyrazolo[3,4-f:3′,4′-j:4″,3″-n][1,4]oxazacyclopentadecine-2,12-diyl]bis(cyclopropylmethanone) (30.0 mg, 0.050 mmol, 1 eq) in MeOH (1 mL) was added K₂CO₃ (20.8 mg, 0.150 mmol, 3 eq). The mixture was stirred at 25° C. for 10 min. The mixture was filtered and concentrated to give a residue. The residue was purified by prep-HPLC to give cyclopropyl[(10S,17E)-16-ethoxy-6,8,10,14,20-pentamethyl-2,8,10,11,13,14-hexahydro-12H-5,3-(azenometheno)tripyrazolo[3,4-f:3′,4′-j:4″,3″-n][1,4]oxazacyclopentadecin-12-yl]methanone (6.78 mg, 0.011 mmol, 22.14% yield, 94.36% purity, FA) as a yellow solid (Ex. 136). ¹H NMR for Ex. 136 can be found in the below NMR table.

### Preparation of 1-[(10S,17E)-6-[(cyclopropyloxy)methyl]-8,10,14,20-tetramethyl-2,8,10,11,13,14-hexahydro-12H-5,3-(azenometheno)tripyrazolo[3,4-f:3′,4′-j:4″,3″-n][1,4]oxazacyclopentadecin-12-yl]-2-(dimethylamino)ethan-1-one (Ex. 138)

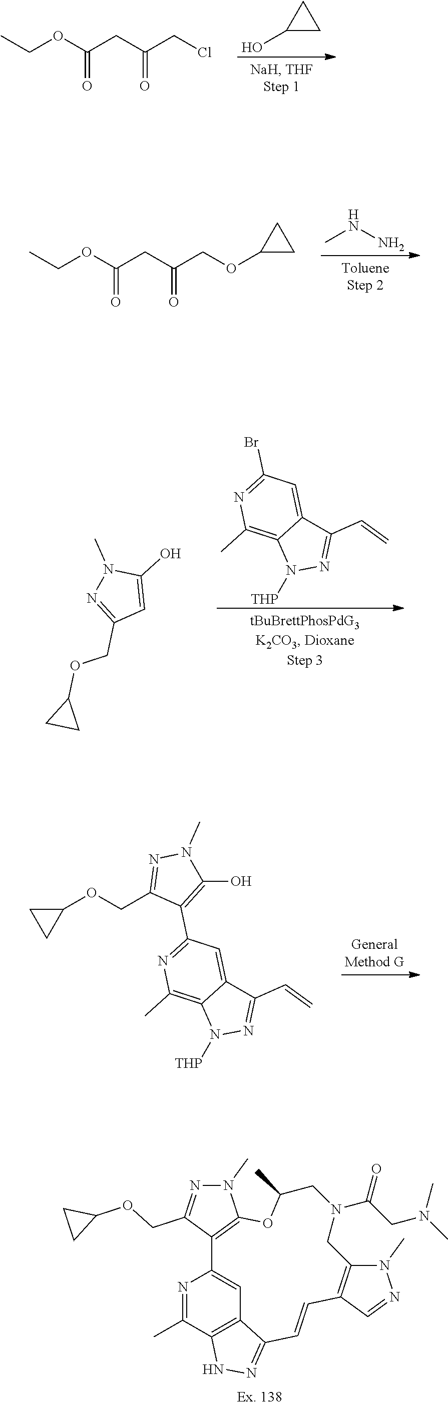

Step 1. To a solution of ethyl 4-chloro-3-oxo-butanoate (21.2 g, 129 mmol, 17.5 mL, 1 eq) in THE (150 mL) was added NaH (15.5 g, 387 mmol, 60% purity, 3 eq) at 0° C. for 30 minutes. And then, cyclopropanol (15.0 g, 258 mmol, 2 eq) was added. The mixture was stirred at 25° C. for 16 h. On completion, the reaction mixture was diluted with H₂O (200 mL) and extracted with Ethyl Acetate (300 mL×3). The combined organic layers were washed with brine (200 mL), dried over Na₂SO₄, filtered, and concentrated under reduced pressure to give a residue. The residue was purified by column chromatography (SiO₂, Petroleum ether/Ethyl acetate=100/1 to 30/1) to give ethyl 4-(cyclopropoxy)-3-oxo-butanoate (15.0 g, 80.5 mmol, 63% yield) as a yellow oil. ¹H NMR (400 MHz, CDCl₃) δ=4.21 (s, 2H), 4.12-4.05 (m, 2H), 3.55 (s, 2H), 3.46-3.38 (m, 1H), 1.21-1.17 (m, 3H), 0.55-0.48 (m, 2H), 0.47-0.40 (m, 2H).

Step 2. To a mixture of methylhydrazine (7.99 g, 69.3 mmol, 9.20 mL, 1.29 eq) in toluene (50 mL) in one portion at 0° C. under N₂, was added a solution ethyl 4-(cyclopropoxy)-3-oxo-butanoate (10.0 g, 53.7 mmol, 1 eq) in toluene (50 mL) drop wise at 0° C. for 30 minutes. The mixture was stirred at 100° C. for 1.5 h. On completion, the reaction mixture was poured into ice-water (100 mL) and stirred for 5 minutes and extracted with Ethyl Acetate (100 mL×3). The combined organic layers were washed with brine (150 mL), dried over Na₂SO₄, filtered, and concentrated under reduced pressure to give a residue. The residue was purified by column chromatography (SiO₂, Petroleum ether/Ethyl acetate=5/1 to 1/1) to give 3-(cyclopropoxymethyl)-1-methyl-1H-pyrazol-5-ol (7.00 g, 41.6 mmol, 77% yield) as a white solid. ¹H NMR (400 MHz, DMSO-d₆) δ=10.81 (s, 1H), 5.30 (s, 1H), 4.21 (s, 2H), 3.45 (s, 3H), 3.31-3.26 (m, 1H), 0.49-0.44 (m, 2H), 0.43-0.38 (m, 2H).

Step 3. A mixture of 5-bromo-7-methyl-1-tetrahydropyran-2-yl-3-vinyl-pyrazolo[3,4-c]pyridine (4.79 g, 14.8 mmol, 1 eq), 5-bromo-7-methyl-1-(tetrahydro-2H-pyran-2-yl)-3-vinyl-1H-pyrazolo[3,4-c]pyridine (500 mg, 1.55 mmol, 1 eq, from Ex. 28), t-BuBrettPhosPdG3 (1.27 g, 1.49 mmol, 0.1 eq), K₂CO₃ (6.16 g, 44.5 mmol, 3 eq) in dioxane (30 mL) was degassed and purged with N₂ for 3 times, and then the mixture was stirred at 130° C. for 1.5 h under N₂ atmosphere. On completion, the reaction mixture was filtered and concentrated under reduced pressure to give a residue. The residue was purified by column chromatography (SiO₂, Petroleum ether/Ethyl acetate=5/1 to 1/1) to give 3-(cyclopropoxymethyl)-1-methyl-4-(7-methyl-1-(tetrahydro-2H-pyran-2-yl)-3-vinyl-1H-pyrazolo[3,4-c]pyridin-5-yl)-1H-pyrazol-5-ol (4.00 g, 8.40 mmol, 56% yield, 86% purity) as a yellow solid. ¹H NMR (400 MHz, DMSO-d₆) δ=7.76 (s, 1H), 7.31-7.19 (m, 1H), 6.05-5.98 (m, 2H), 5.70 (d, J=12.0 Hz, 1H), 4.58 (s, 2H), 3.93 (d, J=11.2 Hz, 1H), 3.82-3.77 (m, 2H), 3.40-3.36 (m, 2H), 2.84 (s, 3H), 2.45-2.38 (m, 1H), 2.13-2.01 (m, 3H), 1.81-1.72 (m, 1H), 1.62 (s, 2H), 1.21-1.10 (m, 1H), 0.56-0.51 (m, 2H), 0.49-0.46 (m, 2H).

Following step 3, Ex. 138 was synthesized from the above intermediate of step 3 following General Method G. ¹H NMR for Ex. 138 can be found in the below NMR table.

### Preparation of (2S)-2-[(10R,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 164) and (2S)-2-[(10S,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (Ex. 165)

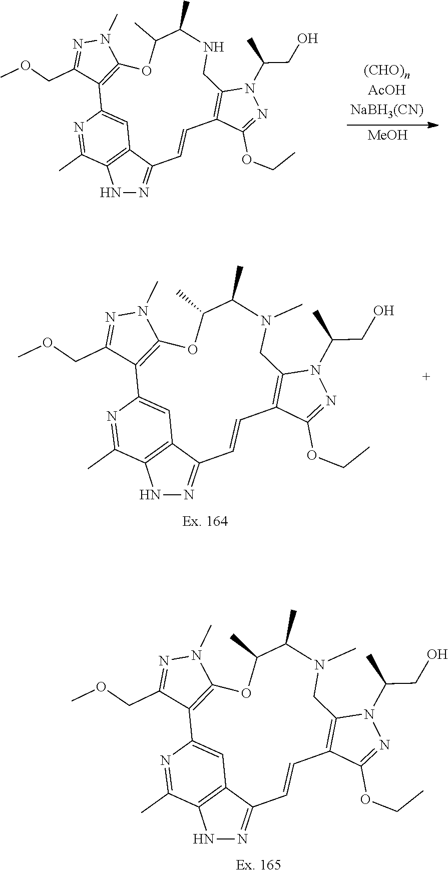 [SMILES: "MeOH" ; CCOc1nn([C@@H](C)CO)c2c1/C=C/c1n[nH]c3c(C)nc(cc13)-c1c(COC)nn(C)c1OC(C)[C@@H](C)NC2 ; CCOc1nn([C@@H](C)CO)c2c1/C=C/c1n[nH]c3c(C)nc(cc13)-c1c(COC)nn(C)c1O[C@H](C)[C@@H](C)N(C)C2 ; CCOc1nn([C@@H](C)CO)c2c1/C=C/c1n[nH]c3c(C)nc(cc13)-c1c(COC)nn(C)c1O[C@@H](C)[C@@H](C)N(C)C2]

To a solution of (2S)-2-[(11R,17E)-16-ethoxy-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol, which was prepared following General Method G (80.0 mg, 0.136 mmol, 1 eq), and Paraformaldehyde (10.0 mg, 0.136 mmol, 1 eq) in MeOH (2 mL) was added AcOH (8.18 mg, 0.136 mmol, 1 eq) and NaBH₃CN (10.2 mg, 0.163 mmol, 1.2 eq). The mixture was stirred at 25° C. for 1 hour. On completion, the mixture was filtered and concentrated to give a residue. The filtrate was purified by prep-HPLC to give (2S)-2-[(10R,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (6.54 mg, 0.010 mmol, 11.76% yield, 97.20% purity, FA salt) as a yellow gum (Ex. 164) and (2S)-2-[(10S,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]propan-1-ol (3.38 mg, 0.0053 mmol, 6.02% yield, 96.25% purity, FA salt) as a yellow gum (Ex. 165). ¹H NMR for Ex. 164 and 165 can be found in the below NMR table.

### Preparation of 2-[(10S,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 170) and 2-[(10R,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 171)

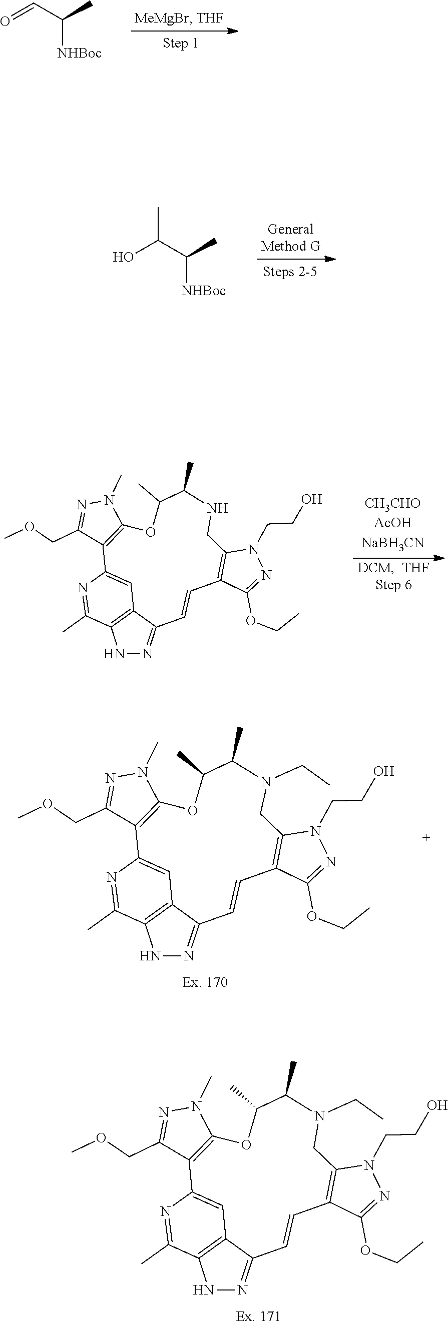 [SMILES: C[C@H](C=O)NC(=O)OC(C)(C)C ; CC(O)[C@@H](C)NC(=O)OC(C)(C)C ; CCOc1nn(CCO)c2c1/C=C/c1n[nH]c3c(C)nc(cc13)-c1c(COC)nn(C)c1OC(C)[C@@H](C)NC2 ; "DCM,  THFStep 6" ; CCOc1nn(CCO)c2c1/C=C/c1n[nH]c3c(C)nc(cc13)-c1c(COC)nn(C)c1O[C@@H](C)[C@@H](C)N(CC)C2 ; CCOc1nn(CCO)c2c1/C=C/c1n[nH]c3c(C)nc(cc13)-c1c(COC)nn(C)c1O[C@H](C)[C@@H](C)N(CC)C2]

Step 1. A mixture of tert-butyl (R)-(1-oxopropan-2-yl)carbamate (5.00 g, 28.8 mmol, 1 eq), bromo(methyl)magnesium (3 M in THF, 19.0 mL, 2 eq) in THE (15 mL) was degassed and purged with N₂, and then the mixture was stirred at −75° C. for 12 hours under N₂ atmosphere. On completion, the residue was diluted with saturated solution of NH₄Cl (30 mL) and extracted with EA (15 mL×3). The combined organic layers were washed with saturated solution of NaCl (15 mL×3), dried over Na₂SO₄, filtered, and concentrated under reduced pressure to give a residue. The residue was purified by column chromatography (SiO₂, Petroleum ether/Ethyl acetate=2:1) to tert-butyl ((2R)-3-hydroxybutan-2-yl)carbamate (2.60 g, 13.7 mmol, 47% yield) as a white solid. ¹H NMR (400 MHz, CDCl₃) δ=4.79 (s, 1H), 3.81-3.71 (m, 1H), 3.63 (d, J=2.8 Hz, 1H), 1.51 (s, 9H), 1.27-1.19 (m, 6H).

Steps 2 through 5 followed the procedures steps 1 through 4 of General Method G using the boc-protected amino alcohol of the previous step and 3-(methoxymethyl)-1-methyl-4-(7-methyl-1-(tetrahydro-2H-pyran-2-yl)-3-vinyl-1H-pyrazolo[3,4-c]pyridin-5-yl)-1H-pyrazol-5-ol from Ex. 140 in step 1 and 2-[5-(bromomethyl)-3-ethoxy-4-iodo-pyrazol-1-yl]ethoxy-tert-butyl-dimethyl-silane from Ex. 1 in step 2.

Step 6. To a solution of 2-[(11R,17E)-16-ethoxy-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (30.0 mg, 0.056 mmol, 1 eq) in DCM (5 mL) and THE (1 mL) was added AcOH (3.36 mg, 0.056 mmol, 1 eq), NaBH₃CN (3.51 mg, 0.056 mmol, 1 eq) and acetaldehyde (2.46 mg, 0.056 mmol, 1 eq). The mixture was stirred at 25° C. for 0.5 hours. On completion, the reaction mixture was filtered, and the filtrate was concentrated under reduced pressure to remove solvent. The crude product was purified by reversed-phase column to give 2-[(10S,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f3′,4′-j:4″,3″-n][1,4]oxazacyclopentadecin-14-yl]ethan-1-ol (7.62 mg, 0.013 mmol, 24.14% yield) as a white solid (Ex. 170) and 2-[(10R,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (5.41 mg, 0.010 mmol, 17.14% yield) as a white solid (Ex. 171). ¹H NMR for Ex. 170 and 171 can be found in the below NMR table.

### Preparation of 2-[(10R,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 174) and 2-[(10S,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (Ex. 175)

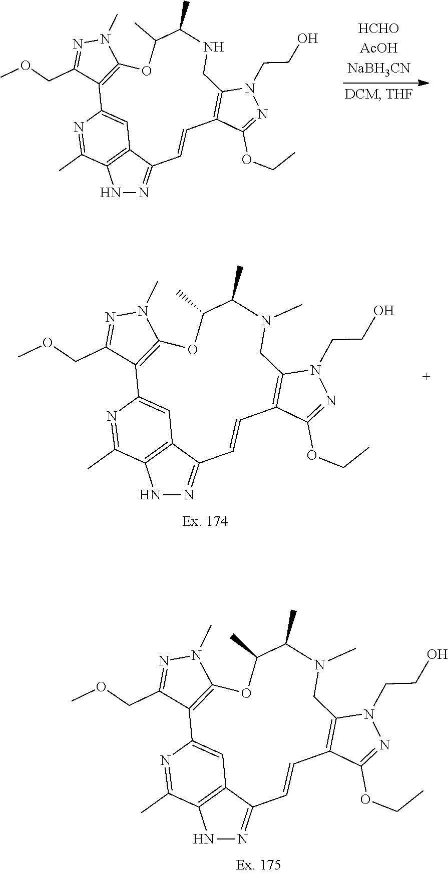 [SMILES: CCOc1nn(CCO)c2c1/C=C/c1n[nH]c3c(C)nc(cc13)-c1c(COC)nn(C)c1OC(C)[C@@H](C)NC2 ; CCOc1nn(CCO)c2c1/C=C/c1n[nH]c3c(C)nc(cc13)-c1c(COC)nn(C)c1O[C@H](C)[C@@H](C)N(C)C2 ; CCOc1nn(CCO)c2c1/C=C/c1n[nH]c3c(C)nc(cc13)-c1c(COC)nn(C)c1O[C@@H](C)[C@@H](C)N(C)C2]

To a solution of 2-[(10S,11R,17E)-16-ethoxy-12-ethyl-6-(methoxymethyl)-8,10,11,20-tetramethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol, which was prepared following General Method G (600 mg, 1.12 mmol, 1 eq, from Ex. 170) in DCM (3 mL) and THE (0.6 mL) was added AcOH (67.1 mg, 1.12 mmol, 1 eq), NaBH₃CN (70.2 mg, 1.12 mmol, 1 eq), and formaldehyde (90.7 mg, 1.12 mmol, 37% purity, 1 eq). The mixture was stirred at 25° C. for 0.5 hours. On completion, the crude product was purified by reversed phase column to give 2-[(10R,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (25.0 mg, 0.045 mmol, 3.99% yield, 98% purity) as a white solid (Ex. 174) and 2-[(10S,11R,17E)-16-ethoxy-6-(methoxymethyl)-8,10,11,12,20-pentamethyl-2,8,10,11,12,13-hexahydro-14H-5,3-(azenometheno)tripyrazolo[3,4-f:3′,4′-j:4″,3″-n][1,4]oxazacyclopentadecin-14-yl]ethan-1-ol (32.3 mg, 0.057 mmol, 5.10% yield, 97% purity) as a white solid (Ex. 175). ¹H NMR for Ex. 174 and 175 can be found in the below NMR table.

### Preparation of {[(10R,17E)-10-(difluoromethyl)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 166) and {[(10S,17E)-10-(difluoromethyl)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 167)

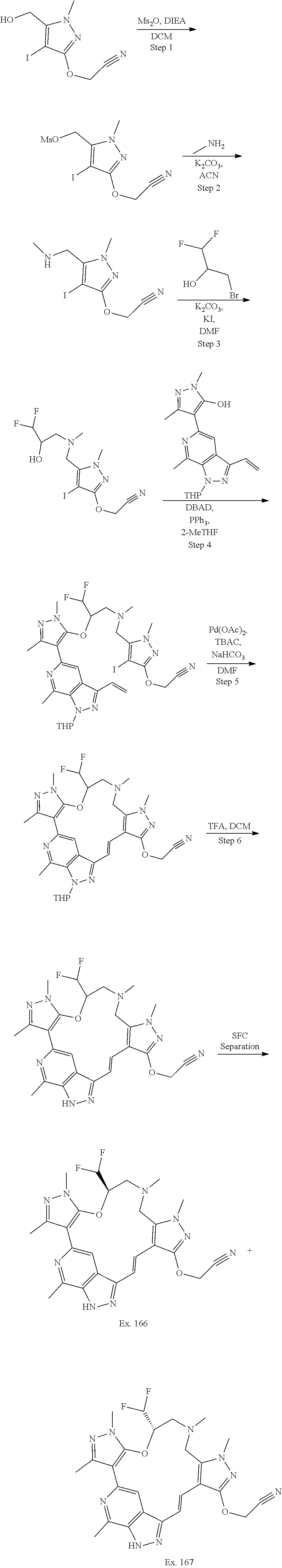

Step 1. To a solution of 2-((5-(hydroxymethyl)-4-iodo-1-methyl-1H-pyrazol-3-yl)oxy)acetonitrile (1.40 g, 4.78 mmol, 1 eq, from Ex. 160) in DCM (20 mL) was added Ms₂O (1.66 g, 9.55 mmol, 2 eq) and DIEA (1.85 g, 14.3 mmol, 3 eq) at 0° C., then the mixture was stirred at 25° C. for 1 h. On completion, the reaction mixture was diluted with H₂O (60 mL) and extracted with DCM (15 mL×3). The combined organic layers were washed with Brine (20 mL×2), dried over Na₂SO₄, filtered, and concentrated under reduced pressure to give a residue. Compound (3-(cyanomethoxy)-4-iodo-1-methyl-1H-pyrazol-5-yl)methyl methanesulfonate (2.00 g, crude) was obtained as a yellow oil. LCMS: (M+1:372.0).

Step 2. To a solution of (3-(cyanomethoxy)-4-iodo-1-methyl-1H-pyrazol-5-yl)methyl methanesulfonate (2.00 g, 5.39 mmol, 1 eq) in ACN (20 mL) was added methanamine hydrochloride (400 mg, 5.93 mmol, 1.1 eq) and K₂CO₃ (2.23 g, 16.2 mmol, 3 eq), then the mixture was stirred at 60° C. for 1 h. On completion, the reaction mixture was concentrated under reduced pressure to give a residue. The residue was purified by flash silica gel chromatography to obtain 2-((4-iodo-1-methyl-5-((methylamino)methyl)-1H-pyrazol-3-yl)oxy)acetonitrile (800 mg, 2.61 mmol, 49% yield) as a yellow solid. LCMS: (M+1:307.0)

Step 3. To a solution of 2-((4-iodo-1-methyl-5-((methylamino)methyl)-1H-pyrazol-3-yl)oxy)acetonitrile (750 mg, 2.45 mmol, 1 eq) in DMF (10 mL) was added 3-bromo-1,1-difluoro-propan-2-ol (857 mg, 4.90 mmol, 2 eq), KT (407 mg, 2.45 mmol, 1 eq) and K₂CO₃(1.02 g, 7.35 mmol, 3 eq), then the mixture was stirred at 80° C. for 16 h. On completion, the reaction mixture was diluted with H₂O (30 mL) and extracted with EA (8 mL×3). The combined organic layers were washed with brine (15 mL×2), dried over Na₂SO₄, filtered, and concentrated under reduced pressure to give a residue. The residue was purified by flash silica gel chromatography (0-50% Ethyl acetate/Petroleum ether). Compound 2-((5-(((3,3-difluoro-2-hydroxypropyl)(methyl)amino)methyl)-4-iodo-1-methyl-1H-pyrazol-3-yl)oxy)acetonitrile (950 mg, 2.37 mmol, 97% yield) was obtained as a yellow oil. LCMS: (M+1:400.8).

Step 4. A mixture of 2-((5-(((3,3-difluoro-2-hydroxypropyl)(methyl)amino)methyl)-4-iodo-1-methyl-1H-pyrazol-3-yl)oxy)acetonitrile (815 mg, 2.04 mmol, 1.2 eq), 1,3-dimethyl-4-(7-methyl-1-(tetrahydro-2H-pyran-2-yl)-3-vinyl-1H-pyrazolo[3,4-c]pyridin-5-yl)-1H-pyrazol-5-ol, which was prepared according to the method described in Ex. 47 (600 mg, 1.70 mmol, 1 eq), DBAD (860 mg, 3.73 mmol, 2.2 eq), PPh₃ (980 mg, 3.73 mmol, 2.2 eq) in 2-MeTHF (15 mL) was degassed and purged with N₂ for 3 times, and then the mixture was stirred at 25° C. for 1 h under N₂ atmosphere. On completion, the reaction mixture was concentrated under reduced pressure to give a residue. The residue was purified by prep-HPLC (neutral condition). Compound 2-((5-(((2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-3,3-difluoropropyl)(methyl)amino)methyl)-4-iodo-1-methyl-1H-pyrazol-3-yl)oxy)acetonitrile (730 mg, 992 mol, 58% yield) was obtained as a yellow solid. LCMS: (M+1:736.2).

Step 5. A mixture of 2-((5-(((2-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-3,3-difluoropropyl)(methyl)amino)methyl)-4-iodo-1-methyl-1H-pyrazol-3-yl)oxy)acetonitrile (700 mg, 0.952 mmol, 1 eq), NaHCO₃ (200 mg, 2.38 mmol, 2.5 eq), TBAC (264 mg, 0.952 mmol, 1 eq) and Pd(OAc)₂ (42.7 mg, 0.190 mmol, 0.2 eq) in DMF (10 mL) was degassed and purged with N₂ for 3 times, and then the mixture was stirred at 130° C. for 1 h under N₂ atmosphere. On completion, the reaction mixture was diluted with H₂O (20 mL) and extracted with EA (10 mL×3). The combined organic layers were washed with brine (20 mL×2), dried over Na₂SO₄, filtered, and concentrated under reduced pressure to give a residue. The residue was purified by flash silica gel chromatography (0-50% Ethyl acetate/Petroleum ether) to afford {[(17E)-10-(difluoromethyl)-6,8,12,14,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (300 mg, 0.494 mmol, 52% yield) as a yellow oil. LCMS: (M+1:608.2).

Step 6. To a solution of {[(17E)-10-(difluoromethyl)-6,8,12,14,20-pentamethyl-2-(oxan-2-yl)-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (270 mg, 0.494 mmol, 1 eq) in DCM (3 mL) was added TFA (8.08 mmol, 0.6 mL, 18.2 eq) and stirred at 25° C. for 1 h. On completion, the reaction mixture was concentrated under reduced pressure to give a residue. The residue was purified by prep-HPLC then separated by Supercritical Fluid Chromatography (SFC) to give compound {[(10R,17E)-10-(difluoromethyl)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 166, 31.81 mg, 0.0558 mmol, 19.49% yield, FA salt) and {[(10S,17E)-10-(difluoromethyl)-6,8,12,14,20-pentamethyl-2,10,11,12,13,14-hexahydro-8H-5,3-(azenometheno)tripyrazolo[3,4-f:3′,4′-j:4″,3″-n][1,4]oxazacyclopentadecin-16-yl]oxy}acetonitrile (Ex. 167, 29.75 mg, 0.0568 mmol, 19.83% yield) as a yellow solid. ¹H NMR for Ex. 166 and 167 can be found in the below NMR table.

### Preparation of (2S)-2-[(4aS,7aS,13E)-12-ethoxy-1,3,8,21-tetramethyl-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol (Ex. 172) and (2S)-2-[(4aR,7aR,13E)-12-ethoxy-1,3,8,21-tetramethyl-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol (Ex. 173)

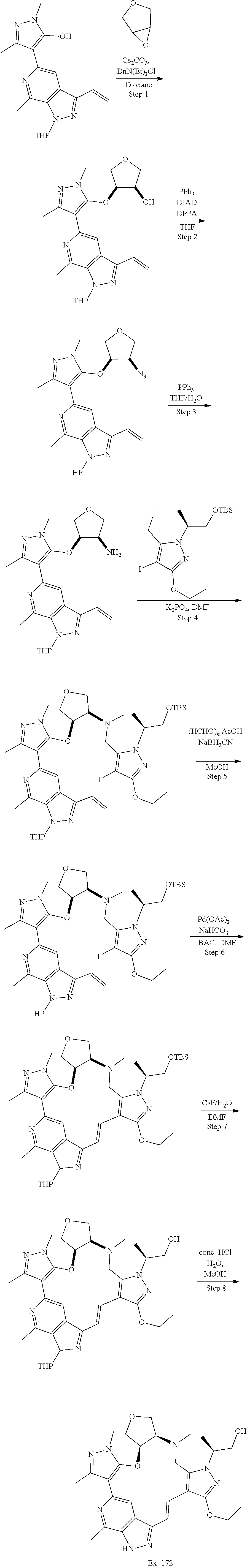

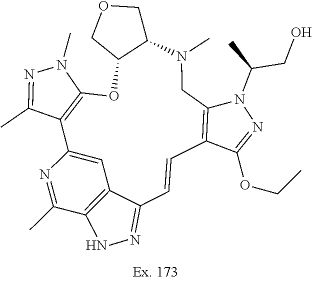 [SMILES: CCOc1nn([C@@H](C)CO)c2c1/C=C/c1n[nH]c3c(C)nc(cc13)-c1c(C)nn(C)c1O[C@@H]1COC[C@@H]1N(C)C2]

Step 1. To a mixture of 1,3-dimethyl-4-(7-methyl-1-(tetrahydro-2H-pyran-2-yl)-3-vinyl-1H-pyrazolo[3,4-c]pyridin-5-yl)-1H-pyrazol-5-ol (5.00 g, 14.2 mmol, 1 eq) in dioxane (50 mL) was added 3,6-dioxabicyclo[3.1.0]hexane (1.22 g, 14.2 mmol, 1 eq), Cs₂CO₃ (6.91 g, 21.2 mmol, 1.5 eq) and benzyl(triethyl)ammonium chloride (644 mg, 2.83 mmol, 0.2 eq), then the mixture was stirred at 120° C. for 48 h. On completion, the mixture was concentrated to give a residue. The residue was purified by column chromatography (SiO₂, PE/THF=1:0 to 1:2) to give (3S,4S)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)tetrahydrofuran-3-ol (2.11 g, 4.80 mmol, 34% yield) as an orange solid. LCMS: (M+1:440.2).

Step 2. To a solution of (3S,4S)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)tetrahydrofuran-3-ol (1.90 g, 4.32 mmol, 1 eq) in THE (95 mL) was added PPh₃ (1.36 g, 5.19 mmol, 1.2 eq), DIAD (1.05 g, 5.19 mmol, 1.2 eq) at 0° C. and the solution was degassed and purged with N₂. [Azido(phenoxy)phosphoryl]oxybenzene (DPPA, 1.43 g, 5.19 mmol, 1.2 eq) was then added. The mixture was stirred at 0° C. for 18 h. On completion, the mixture was concentrated to give a residue. The residue was purified by column chromatography (SiO₂, PE:THF=1:0 to 1:1) to give 5-(5-(((3R,4R)-4-azidotetrahydrofuran-3-yl)oxy)-1,3-dimethyl-1H-pyrazol-4-yl)-7-methyl-1-(tetrahydro-2H-pyran-2-yl)-3-vinyl-1H-pyrazolo[3,4-c]pyridine (2.62 g, 2.31 mmol, 53% yield, 41% purity) as a yellow solid. LCMS: (M+1:465.4).

Step 3. To a solution of 5-(5-(((3R,4R)-4-azidotetrahydrofuran-3-yl)oxy)-1,3-dimethyl-1H-pyrazol-4-yl)-7-methyl-1-(tetrahydro-2H-pyran-2-yl)-3-vinyl-1H-pyrazolo[3,4-c]pyridine (2.58 g, 5.55 mmol, 1 eq) in THE (24 mL) and H₂O (8 mL) was added PPh₃ (1.75 g, 6.66 mmol, 1.2 eq). The mixture was stirred at 25° C. for 16 h. On completion, the mixture was quenched with water (15 mL) and extracted with 2-MeTHF (15 mL×3), the combined organic phase was dried over anhydrous sodium sulfate, filtered, and concentrated to give a residue. The residue was purified by column chromatography (SiO₂, DCM/MeOH=1:0 to 10:1) to give (3R,4R)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)tetrahydrofuran-3-amine (510 mg, 1.16 mmol, 21% yield) as a colorless oil. LCMS: (M+1:439.2).

Step 4. To a solution of (3R,4R)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)tetrahydrofuran-3-amine (450 mg, 1.03 mmol, 1 eq) in DMF (5 mL) was added (S)-1-(1-((tert-butyldimethylsilyl)oxy)propan-2-yl)-3-ethoxy-4-iodo-5-(iodomethyl)-1H-pyrazole, which was prepared according to the method described in Ex. 120 (621 mg, 1.13 mmol, 1.1 eq) and K₃PO₄ (436 mg, 2.05 mmol, 2 eq). The mixture was stirred at 60° C. for 3 h. On completion, the mixture was quenched with water (15 mL) and extracted with ethyl acetate (10 mL×3), the combined organic phase was dried over anhydrous sodium sulfate, filtered, and concentrated to give a residue. The residue was purified by column chromatography (SiO₂, PE/THF=1:0 to 2:1) to give (3R,4R)-N-((1-((S)-1-((tert-butyldimethylsilyl)oxy)propan-2-yl)-3-ethoxy-4-iodo-1H-pyrazol-5-yl)methyl)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)tetrahydrofuran-3-amine (710 mg, 0.825 mmol, 80% yield) as a yellow oil. LCMS: (M+1:861.4).

Step 5. To a solution of (3R,4R)-N-((1-((S)-1-((tert-butyldimethylsilyl)oxy)propan-2-yl)-3-ethoxy-4-iodo-1H-pyrazol-5-yl)methyl)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)tetrahydrofuran-3-amine (650 mg, 0.755 mmol, 1 eq) and (HCHO)ₙ (680 mg, 7.55 mmol, 10 eq) in MeOH (7 mL) was added AcOH (45.3 mg, 0.755 mmol, 1 eq), and stirred at 25° C. for 30 mins, then NaBH₃CN (94.9 mg, 1.51 mmol, 2 eq) was added. The mixture was stirred at 25° C. for 16 h. On completion, the mixture was concentrated to give a residue. The residue was purified by column chromatography (SiO₂, PE/EA=1:0 to 1:2) to give (3R,4R)-N-((1-((S)-1-((tert-butyldimethylsilyl)oxy)propan-2-yl)-3-ethoxy-4-iodo-1H-pyrazol-5-yl)methyl)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methyltetrahydrofuran-3-amine (130 mg, 0.177 mmol, 53% yield) as a yellow oil. LCMS: (M+1:876.0).

Step 6. To a mixture of (3R,4R)-N-((1-((S)-1-((tert-butyldimethylsilyl)oxy)propan-2-yl)-3-ethoxy-4-iodo-1H-pyrazol-5-yl)methyl)-4-((1,3-dimethyl-4-(7-methyl-1-(tetrahydro-2H-pyran-2-yl)-3-vinyl-1H-pyrazolo[3,4-c]pyridin-5-yl)-1H-pyrazol-5-yl)oxy)-N-methyltetrahydrofuran-3-amine (465 mg, 0.531 mmol, 1 eq) in DMF (5 mL) was added NaHCO₃ (112 mg, 1.33 mmol, 2.5 eq), TBAC (148 mg, 0.531 mmol, 1 eq) and Pd(OAc)₂ (23.9 mg, 0.106 mmol, 0.2 eq). The mixture was degassed and purged with N₂ thrice, and then the mixture was stirred at 130° C. for 1 h under N₂ atmosphere. On completion, (13E)-10-[(2S)-1-{[tert-butyl(dimethyl)silyl]oxy}propan-2-yl]-12-ethoxy-1,3,8,21-tetramethyl-16-(oxan-2-yl)-4a,5,7,7a,8,9,10,16-octahydro-3H-19,17-(azenometheno)furo[3,4-b|tripyrazolol3,4-f:3′,4′-j:4″,3″-n∥1,4]oxazacyclopentadecine (465 mg, crude in DMF) was obtained as a brown solid. LCMS: (M+1:747.5).

Step 7. A mixture of (13E)-10-[(2S)-1-{[tert-butyl(dimethyl)silyl]oxy}propan-2-yl]-12-ethoxy-1,3,8,21-tetramethyl-16-(oxan-2-yl)-4a,5,7,7a,8,9,10,16-octahydro-3H-19,17-(azenometheno)furo[3,4-b]tripyrazolo[3,4-f:3′,4′-j:4″,3″-n][1,4]oxazacyclopentadecine (465 mg, crude in DMF) was added Cesium Fluoride (284 mg, 1.87 mmol, 3 eq) and H₂O (11.2 mg, 0.622 mmol, 1 eq), and then the mixture was stirred at 30° C. for 16 h. On completion, the mixture was quenched with water (15 mL) and extracted with ethyl acetate (15 mL×3), and the combined organic phase was dried over anhydrous sodium sulfate, filtered and concentrated to give a residue. The residue was purified by column chromatography (SiO₂, PE/THF=1:0 to 1:3) to give (2S)-2-[(13E)-12-ethoxy-1,3,8,21-tetramethyl-16-(oxan-2-yl)-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol (200 mg, 0.316 mmol, 51% yield) as a brown solid. LCMS: (M+1:633.3)

Step 8. To a solution of (2S)-2-[(13E)-12-ethoxy-1,3,8,21-tetramethyl-16-(oxan-2-yl)-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol (180 mg, 0.284 mmol, 1 eq) in MeOH (0.6 mL) was added conc. HCl (12 M, 0.2 mL). The mixture was stirred at 25° C. for 30 mins. On completion, the mixture was purified by prep-HPLC to give (2S)-2-[(4aS,7aS,13E)-12-ethoxy-1,3,8,21-tetramethyl-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol (3.5 mg, 0.0064 mmol, 2.24% yield, Formic acid salt) as a white solid (Ex. 172) and (2S)-2-[(4aR,7aR,13E)-12-ethoxy-1,3,8,21-tetramethyl-3,4a,5,7,7a,8,9,16-octahydro-10H-19,17-(azenometheno)furo[3,4-b]tripyrazolo[3,4-f:3′,4′-j:4″,3″-n][1,4]oxazacyclopentadecin-10-yl]propan-1-ol (4.2 mg, 0.0077 mmol, 2.69% yield, Formic acid salt) as a white solid (Ex. 173).

Ex. 172: LCMS: (M+1:549.1). ¹H NMR for Ex. 172 can be found in the below NMR table.

Ex. 173: LCMS: (M+1:549.1). ¹H NMR for Ex. 173 can be found in the below NMR table.

The following examples were synthesized following General Method A or B or C or D or E or F or G as indicated in the NMR Table:

| General | | | MS m/z | |
|---|---|---|---|---|
| Method | Ex. | Structure | [M + H]⁺ | ¹H NMR |
| A | 1 | 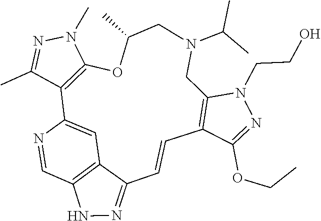 | 521.3 | ¹H NMR (400 MHz, MeOD-d₄) δ = 8.87 (s, 1H), 8.46 (s, 1H), 7.98 (br d, J = 16.8 Hz, 1H), 7.19 (d, J = 16.8 Hz, 1H), 4.38-4.30 (m, 2H), 4.18-4.08 (m, 2H), 4.03- 3.92 (m, 2H), 3.91- 3.84 (m, 2H), 3.66 (s, 3H), 2.91-2.75 (m, 3H), 2.52 (s, 3H), 1.49 (t, J = 7.2 Hz, 3H), 1.04 (br d, J = 6.4 Hz, 3H), 0.94 (br d, J = 6.4 Hz, 3H), 0.89 (br d, J = 6.4 Hz, 3H). |
| D | 2 | 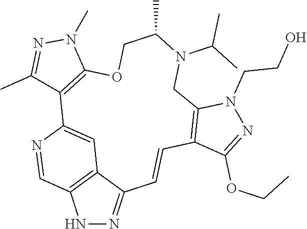 [SMILES: CCOc1nn(CCO)c2c1/C=C/c1n[nH]c3cnc(cc13)-c1c(C)nn(C)c1OC[C@H](C)N(C(C)C)C2] | 521.2 | ¹H NMR (400 MHz, MeOD-d₄) δ = 8.93 (s, 1H), 8.57 (s, 1H), 7.75 (br d, J = 16.4 Hz, 1H), 7.33 (d, J = 16.8 Hz, 1H), 4.51-4.42 (m, 1H), 4.41-4.32 (m, 2H), 4.32- 4.23 (m, 1H), 4.15- 4.06 (m, 1H), 3.99 (s, 2H), 3.96-3.83 (m, 3H), 3.75 (s, 3H), 3.48-3.41 (m, 1H), 3.09-2.98 (m, 1H), 2.59 (s, 3H), 1.50 (t, J = 7.2 Hz, 3H), 1.42 (d, J = 7.2 Hz, 3H), 1.14 (br d, J = 6.4 Hz, 3H), 1.01 (d, J = 6.8 Hz, 3H). |
| D | 3 | 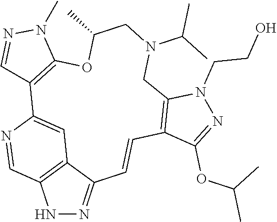 [SMILES: CC(C)Oc1nn(CCO)c2c1/C=C/c1n[nH]c3cnc(cc13)-c1cnn(C)c1O[C@H](C)CN(C(C)C)C2] | 521.3 | ¹H NMR (400 MHz, MeOD-d₄) δ = 9.04 (s, 1H), 8.59 (br s, 1H), 8.11 (s, 1H), 7.53 (s, 1H), 7.48 (br s, 1H), 5.18-5.01(m, 2H), 4.71- 4.52 (m, 2H), 4.37- 4.17 (m, 3H), 4.07-3.92 (m, 3H), 3.87 (s, 3H), 3.36-3.32 (m, 1H), 1.74- 1.56 (m, 5H), 1.49 (t, J = 6.4 Hz, 6H), 1.36 (br d, J = 5.6 Hz, 3H). |
| D | 4 | 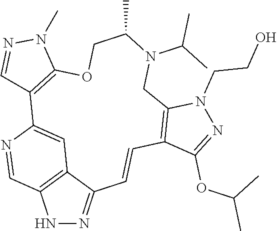 [SMILES: CC(C)Oc1nn(CCO)c2c1/C=C/c1n[nH]c3cnc(cc13)-c1cnn(C)c1OC[C@H](C)N(C(C)C)C2] | 521.3 | ¹H NMR (400 MHz, MeOD-d₄) δ = 9.08 (d, J = 0.8 Hz, 1H), 8.79 (s, 1H), 8.08 (s, 1H), 7.64 (br d, J = 15.2 Hz, 1H), 7.43(d, J = 16.4 Hz, 1H), 5.34 (br s, 1H), 5.12- 5.03 (m, 1H), 4.61 (br s, 2H), 4.29-4.18 (m, 1H), 4.16-4.07 (m, 1H), 4.04- 3.95 (m, 1H), 3.91- 3.83 (m, 1H), 3.81 (s, 3H), 3.75 (br d, J = 12.0 Hz, 1H), 3.29-3.24 (m, 1H), 1.52-1.38 (m, 12H), 1.24 (d, J = 6.4 Hz, 3H). |
| A | 5 | 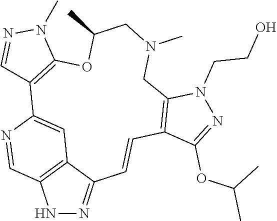 [SMILES: CC(C)Oc1nn(CCO)c2c1/C=C/c1n[nH]c3cnc(cc13)-c1cnn(C)c1O[C@@H](C)CN(C)C2] | 493.1 | ¹H NMR (400 MHz, MeOD-d₄) δ = 8.88 (s, 1H), 8.49 (s, 1H), 8.37 (br d, J = 16.0 Hz, 1H), 8.05 (s, 1H), 7.32 (d, J = 16.0 Hz, 1H), 5.02-4.96 (m, 1H), 4.92-4.89 (m, 1H), 4.11 (td, J = 4.4, 8.0 Hz, 2H), 4.03-3.97 (m, 1H), 3.88 (br d, J = 12.0 Hz, 1H), 3.86-3.81 (m, 2H), 3.75 (s, 3H), 3.01-2.87 (m, 2H), 2.47 (s, 3H), 1.45 (t, J = 6.4 Hz, 6H), 1.17 (d, J = 6.4 Hz, 3H). |
| A | 6 | 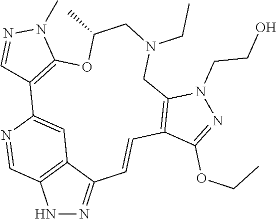 [SMILES: CCOc1nn(CCO)c2c1/C=C/c1n[nH]c3cnc(cc13)-c1cnn(C)c1O[C@H](C)CN(CC)C2] | 493.2 | ¹H NMR (400 MHz, DMSO-d₆) δ = 13.30 (s, 1H), 8.94 (s, 1H), 8.45 (s, 1H), 8.11 (d, J = 17.2 Hz, 1H), 7.96 (s, 1H), 7.16 (d, J = 17.2 Hz, 1H), 4.76 (s, 1H), 4.28 (d, J = 6.8 Hz, 2H), 4.04 (d, J = 5.6 Hz, 2H), 4.00- 3.87 (m, 2H), 3.70 (s, 3H), 3.65 (s, 2H), 3.04- 2.87 (m, 2H), 2.76-2.65 (m, 1H), 2.64-2.56 (m, 1H), 1.41 (t, J = 6.8 Hz, 3H), 1.34-1.20 (m, 1H), 1.16-1.08 (m, 3H), 0.96 (t, J = 7.2 Hz, 3H). |
| A | 7 | 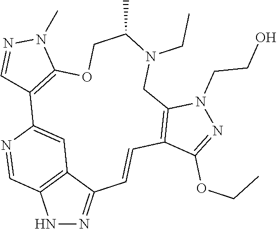 [SMILES: CCOc1nn(CCO)c2c1/C=C/c1n[nH]c3cnc(cc13)-c1cnn(C)c1OC[C@H](C)N(CC)C2] | 493.1 | ¹H NMR (400 MHz, DMSO-d₆) δ = 13.40- 13.25 (m, 1H), 8.93 (s, 1H), 8.45 (s, 1H), 8.34 (s, 1H), 7.91 (s, 1H), 7.59 (d, J = 16.4 Hz, 1H), 7.17 (d, J = 16.8 Hz, 1H), 4.96-4.85 (m, 1H), 4.55 (d, J = 4.4, 8.8 Hz, 1H), 4.33-4.23 (m, 2H), 4.20-4.10 (m, 1H), 4.06- 3.94 (m, 3H), 3.81 (d, J = 14.8 Hz, 1H), 3.74 (s, 3H), 3.70 (d, J = 5.6 Hz, 2H), 2.77 (d, J = 6.4, 13.5 Hz, 1H), 2.69-2.65 (m, 1H), 1.44-1.38 (m, 3H), 1.29 (d, J = 6.8 Hz, 3H), 1.03 (t, J = 6.8 Hz, 3H). |
| A | 8 | 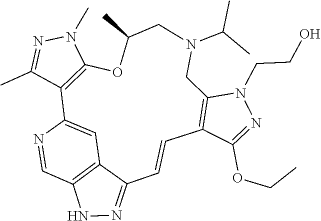 | 521.3 | ¹H NMR (400 MHz, MeOD-d₄) δ = 8.94 (s, 1H), 8.59 (s, 1H), 8.10 (br d, J = 16.8 Hz, 1H), 7.26 (d, J = 16.8 Hz, 1H),4.65 (br d, J = 5.2 Hz, 1H), 4.40-4.31 (m, 2H), 4.18-4.12 (m, 2H), 4.11-4.00 (m, 2H), 3.88- 3.82 (m, 2H), 3.71 (s, 3H), 2.93-2.82 (m, 3H), 2.55 (s, 3H), 1.50 (t, J = 7.2 Hz, 3H), 1.11 (d, J = 6.4 Hz, 3H), 1.02-0.93 (m, 6H). |
| D | 9 | 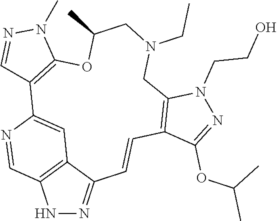 [SMILES: CCN1Cc2c(c(OC(C)C)nn2CCO)/C=C/c2n[nH]c3cnc(cc23)-c2cnn(C)c2O[C@@H](C)C1] | 507.3 | ¹H NMR (400 MHz, MeOD-d₄) δ = 9.03 (s, 1H), 8.70 (br s, 1H), 8.08 (s, 1H), 7.86-7.52 (m, 1H), 7.42 (d, J = 16.4 Hz, 1H), 5.36-5.19 (m, 1H), 5.14-5.01 (m, 1H), 4.71-4.61 (m, 2H), 4.51-4.31 (m, 1H), 4.26- 4.13 (m, 2H), 4.01- 3.83 (m, 3H), 3.81 (s, 3H), 3.23-3.04 (m, 2H), 1.51-1.33 (m, 9H), 1.25 (br d, J = 6.4 Hz, 3H). |
| D | 10 | 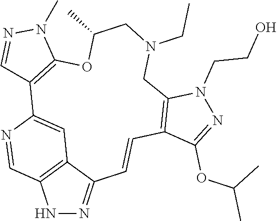 [SMILES: CCN1Cc2c(c(OC(C)C)nn2CCO)/C=C/c2n[nH]c3cnc(cc23)-c2cnn(C)c2O[C@H](C)C1] | 507.3 | ¹H NMR (400 MHz, MeOD-d₄) δ = 9.05- 8.98 (m, 1H), 8.74-8.65 (m, 1H), 8.08 (s, 1H), 7.89-7.58 (m, 1H), 7.41 (d, J = 16.4 Hz, 1H), 5.35-5.17 (m, 1H), 5.12- 4.98 (m, 1H), 4.70- 4.57 (m, 2H), 4.50-4.32 (m, 1H), 4.27-4.13 (m, 2H), 4.03-3.82 (m, 3H), 3.80 (s, 3H), 3.23-3.07 (m, 2H), 1.50-1.33 (m, 9H), 1.24 (br d, J = 6.4 Hz, 3H). |
| A | 11 | 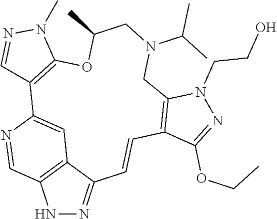 [SMILES: CCOc1nn(CCO)c2c1/C=C/c1n[nH]c3cnc(cc13)-c1cnn(C)c1O[C@@H](C)CN(C(C)C)C2] | 507.1 | ¹H NMR (400 MHz, MeOD-d₄) δ = 8.88 (s, 1H), 8.69 (br s, 1H), 8.14-8.07 (m, 1H), 8.06 (s, 1H), 7.31 (d, J = 16.0 Hz, 1H), 4.83 (br s, 1H), 4.36 (q, J = 8.0 Hz, 2H), 4.25-4.11 (m, 2H), 4.01 (s, 2H), 3.85 (t, J = 6.0 Hz, 2H), 3.78 (s, 3H), 3.00 (br s, 1H), 2.96- 2.85 (m, 2H), 1.50 (t, J = 8.0 Hz, 3H), 1.22 (br d, J = 6.0 Hz, 3H), 1.05 (br dd, J = 8.0, 16.0 Hz, 6H) |
| A | 12 | 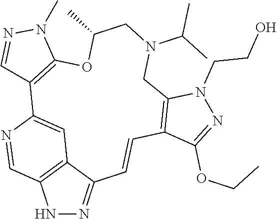 [SMILES: CCOc1nn(CCO)c2c1/C=C/c1n[nH]c3cnc(cc13)-c1cnn(C)c1O[C@H](C)CN(C(C)C)C2] | 507.2 | ¹H NMR (400 MHz, MeOD-d₄) δ = 8.87 (s, 1H), 8.68 (br s, 1H), 8.14-8.06 (m, 1H), 8.05 (s, 1H), 7.31 (d, J = 16.0 Hz, 1H), 4.81 (br s, 1H), 4.35 (q, J = 8.0 Hz, 2H), 4.23-4.11 (m, 2H), 4.00 (s, 2H), 3.85 (t, J = 6.0 Hz, 2H), 3.77 (s, 3H), 3.04-2.96 (m, 1H), 2.95- 2.84 (m, 2H), 1.50 (t, J = 8.0 Hz, 3H), 1.21 (br d, J = 8.0 Hz, 3H), 1.04 (br dd, J = 6.0, 16.0 Hz, 6H). |
| A | 13 | 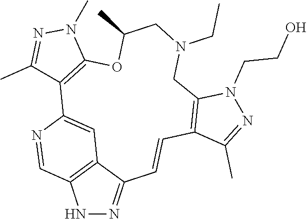 [SMILES: CCN1Cc2c(c(C)nn2CCO)/C=C/c2n[nH]c3cnc(cc23)-c2c(C)nn(C)c2O[C@@H](C)C1] | 477.1 | ¹H NMR (400 MHz, MeOD-d₄) δ = 8.52 (s, 1H), 7.98 (s, 1H), 7.84- 7.75 (m, 1H), 6.54 (d, J = 17.2 Hz, 1H), 4.65- 4.57 (m, 4H), 4.26-4.18 (m, 1H), 3.95-3.80 (m, 2H), 3.67-3.55 (m, 4H), 3.35-3.32 (m, 1H), 2.65- 2.58 (m, 1H), 2.51- 2.43 (m, 1H), 2.19-2.13 (m, 6H), 0.65 (br d, J = 6.4 Hz, 3H), 0.55-0.48 (m, 3H). |
| A | 14 | 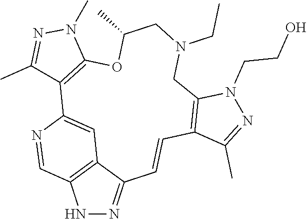 [SMILES: CCN1Cc2c(c(C)nn2CCO)/C=C/c2n[nH]c3cnc(cc23)-c2c(C)nn(C)c2O[C@H](C)C1] | 477.1 | ¹H NMR (400 MHz, MeOD-d₄) δ = 8.56 (s, 1H), 8.09 (s, 1H), 7.91 (br d, J = 17.2 Hz, 1H), 6.60 (d, J = 17.6 Hz, 1H), 4.20 (br s, 1H), 3.92-3.78 (m, 2H), 3.65 (br d, J = 16.0 Hz, 2H), 3.50 (t, J = 5.2 Hz, 2H), 3.31 (s, 3H), 2.94 (s, 2H), 2.68-2.61 (m, 1H), 2.52-2.44 (m, 1H), 2.17- 2.11 (m, 6H), 0.69 (d, J = 6.4 Hz, 3H), 0.57- 0.52 (m, 3H). |
| A | 15 | 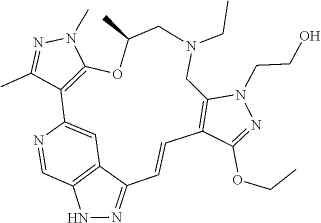 | 507.2 | ¹H NMR (400 MHz, DMSO-d₆) δ = 13.20 (s, 1H), 8.96 (s, 1H), 8.48 (s, 1H), 8.06 (d, J = 17.2 Hz, 1H), 7.15 (d, J = 17.2 Hz, 1H), 4.91-4.61 (m, 2H), 4.34-4.27 (m, 2H), 4.11-3.89 (m, 5H), 3.69-3.65 (m, 2H), 3.63 (s, 3H), 2.93 (d, J = 3.2 Hz, 2H), 2.69-2.54 (m, 4H), 1.42 (t, J = 7.2 Hz, 3H), 1.07 (d, J = 6.4 Hz, 3H), 0.93 (t, J = 7.2 Hz, 3H). |
| A | 16 | 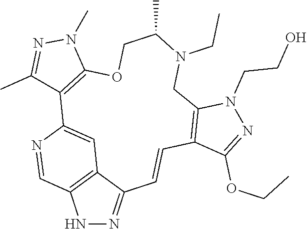 [SMILES: CCOc1nn(CCO)c2c1/C=C/c1n[nH]c3cnc(cc13)-c1c(C)nn(C)c1OC[C@H](C)N(CC)C2] | 507.3 | ¹H NMR (400 MHz, MeOD-d₄) δ = 9.11- 8.97 (m, 1H), 8.66-8.52 (m, 1H), 7.60-7.45 (m, 1H), 7.43-7.30 (m, 1H), 5.38-5.19 (m, 1H), 4.83- 4.70 (m, 1H), 4.63- 4.50 (m, 1H), 4.48-4.34 (m, 2H), 4.31-4.13 (m, 2H), 4.04-3.93 (m, 1H), 3.92-3.78 (m, 2H), 3.77- 3.71 (m, 3H), 3.57- 3.38 (m, 2H), 2.61-2.51 (m, 3H), 1.55-1.38 (m, 6H), 1.24-1.14 (m, 3H). |
| A | 17 | 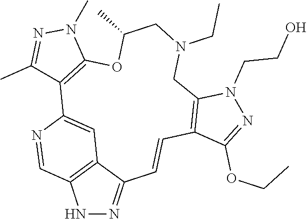 [SMILES: CCOc1nn(CCO)c2c1/C=C/c1n[nH]c3cnc(cc13)-c1c(C)nn(C)c1O[C@H](C)CN(CC)C2] | 507.3 | ¹H NMR (400 MHz, MeOD-d₄) δ = 8.94 (s, 1H), 8.65-8.43 (m, 1H), 8.27-8.09 (m, 1H), 7.31- 7.18 (m, 1H), 4.80- 4.68 (m, 1H), 4.40-4.28 (m, 2H), 4.21-4.08 (m, 2H), 4.07-3.94 (m, 2H), 3.88-3.79 (m, 2H), 3.73- 3.65 (m, 3H), 3.08- 2.98 (m, 1H), 2.96-2.85 (m, 1H), 2.76-2.62 (m, 2H), 2.58-2.49 (m, 3H), 1.50 (t, J = 7.2 Hz, 3H), 1.11 (d, J = 6.4 Hz, 3H), 0.98 (t, J = 7.2 Hz, 3H). |
| A | 18 | 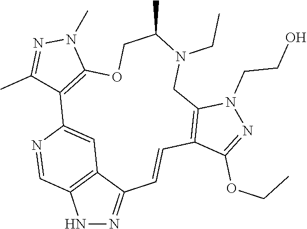 [SMILES: CCOc1nn(CCO)c2c1/C=C/c1n[nH]c3cnc(cc13)-c1c(C)nn(C)c1OC[C@@H](C)N(CC)C2] | 507.2 | ¹H NMR (400 MHz, DMSO-d₆) δ = 13.40- 13.13 (m, 1H), 8.96 (s, 1H), 8.41-8.37 (m, 1H), 7.56 (d, J = 16.8 Hz, 1H), 7.18 (d, J = 16.8 Hz, 1H), 4.93-4.87 (m, 1H), 4.59-4.51 (m, 1H), 4.33-4.24 (m, 2H), 4.19- 4.10 (m, 1H), 4.07- 3.93 (m, 3H), 3.83-3.77 (m, 1H), 3.70 (d, J = 6.0 Hz, 2H), 3.68 (s, 3H), 3.24-3.17 (m, 1H), 2.81- 2.71 (m, 1H), 2.52 (s, 3H), 2.45-2.38 (m, 1H), 1.41 (t, J = 7.2 Hz, 3H), 1.28 (d, J = 6.8 Hz, 3H), 1.03 (t, J = 6.8 Hz, 3H). |
| A | 19 | 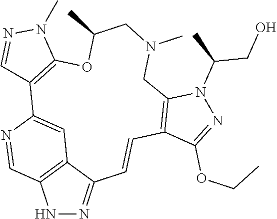 [SMILES: CCOc1nn([C@@H](C)CO)c2c1/C=C/c1n[nH]c3cnc(cc13)-c1cnn(C)c1O[C@@H](C)CN(C)C2] | 493.1 | ¹H NMR (400 MHz, MeOD-d₄) δ = 8.86 (s, 1H), 8.45 (br s, 1H), 8.30 (br d, J = 16.0 Hz, 1H), 8.03 (s, 1H), 7.27 (br d, J = 16.0 Hz, 1H), 4.49-4.41 (m, 1H), 4.36 (br dd, J = 4.0, 8.0 Hz, 2H), 3.99 (br d, J = 12.0 Hz, 1H), 3.88-3.79 (m, 2H), 3.74 (s, 3H), 3.69 (br dd, J = 4.4, 12.0 Hz, 1H), 3.35 (s, 1H), 2.97- 2.86 (m, 2H), 2.44 (s, 3H), 1.48 (br t, J = 8.0 Hz, 3H), 1.40 (br d, J = 6.0 Hz, 3H), 1.15 (br d, J = 6.0 Hz, 3H) |
| A | 20 | 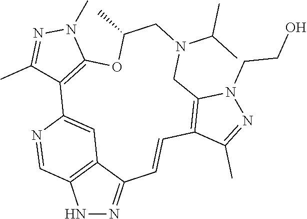 [SMILES: Cc1nn(CCO)c2c1/C=C/c1n[nH]c3cnc(cc13)-c1c(C)nn(C)c1O[C@H](C)CN(C(C)C)C2] | 491.1 | ¹H NMR (400 MHz, MeOD-d₄) δ = 8.92 (br s, 1H), 8.49 (br s, 1H), 8.17 (br s, 1H), 6.97 (br d, J = 17.2 Hz, 1H), 4.69- 4.47 (m, 2H), 4.27- 4.19 (m, 2H), 4.14-4.01 (m, 2H), 3.91-3.85 (m, 2H), 3.69 (s, 3H), 3.35 (s, 1H), 2.91-2.79 (m, 2H), 2.51 (br d, J = 12.0 Hz, 6H), 1.31-1.22 (m, 3H), 1.07-1.00 (m, 3H), 0.97-0.91 (m, 3H). |
| A | 21 | 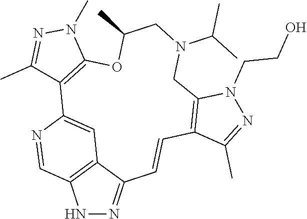 [SMILES: Cc1nn(CCO)c2c1/C=C/c1n[nH]c3cnc(cc13)-c1c(C)nn(C)c1O[C@@H](C)CN(C(C)C)C2] | 491.2 | ¹H NMR (400 MHz, MeOD-d₄) δ = 9.08 (s, 1H), 8.62 (s, 1H), 7.77- 7.63 (m, 1H), 7.24-7.13 (m, 1H), 5.51-5.17 (m, 1H), 4.80-4.72 (m, 2H), 4.38-4.30 (m, 1H), 4.23- 4.14 (m, 1H), 4.08- 3.99 (m, 1H), 3.93-3.82 (m, 2H), 3.73 (s, 4H), 3.29-3.22 (m, 1H), 2.56 (d, J = 13.2 Hz, 7H), 1.64-1.40 (m, 6H), 1.17 (d, J = 6.4 Hz, 3H). |
| B | 22 | 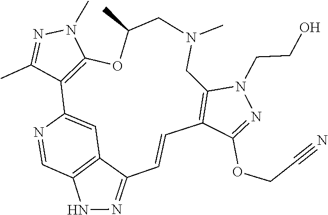 | 504.1 | ¹H NMR (400 MHz, MeOH-d₄) δ = 9.00 (d, J = 1.2 Hz, 1H), 8.47 (s, 1H), 8.18-7.99 (m, 1H), 7.21 (d, J = 16.8 Hz, 1H), 5.18 (s, 2H), 4.98- 4.92 (m, 1H), 4.25-4.03 (m, 4H), 3.93-3.85 (m, 2H), 3.70 (s, 3H), 3.18- 3.03 (m, 2H), 2.68-2.49 (m, 6H), 1.13 (d, J = 6.4 Hz, 3H). |
| C | 23 | 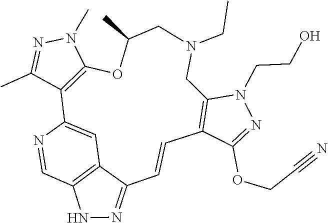 | 518.2 | ¹H NMR (400 MHz, MeOD-d₄) δ = 9.02 (d, J = 1.2 Hz, 1H), 8.55 (s, 1H), 7.85-7.69 (m, 1H), 7.27 (d, J = 16.4 Hz, 1H), 5.21 (s, 2H), 5.14- 5.04 (m, 1H), 4.60-4.35 (m, 2H), 4.27-4.20 (m, 2H), 3.98-3.87 (m, 2H), 3.72 (s, 3H), 3.64-3.45 (m, 1H), 3.27-3.00 (m, 3H), 2.55 (s, 3H), 1.29 (br s, 3H), 1.16 (d, J = 6.4 Hz, 3H). |
| A | 24 | 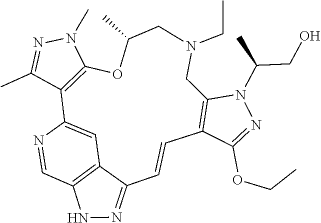 | 521.3 | ¹H NMR (400 MHz, MeOD-d₄) δ = 8.94 (s, 1H), 8.26-8.21 (m, 1H), 7.19-7.11 (m, 1H), 6.07- 6.01 (m, 1H), 5.28- 5.22 (m, 1H), 4.79 (br dd, J = 6.8, 12.4 Hz, 2H), 4.05-4.01 (m, 1H), 4.01-3.99 (m, 1H), 3.97- 3.94 (m, 1H), 3.93- 3.89 (m, 1H), 3.87-3.85 (m, 1H), 3.82-3.77 (m, 1H), 3.64-3.63 (m, 3H), 3.53-3.45 (m, 1H), 2.81- 2.72 (m, 1H), 2.56- 2.50 (m, 1H), 2.43-2.40 (m, 3H), 1.48 (d, J = 6.4 Hz, 3H), 1.28-1.20 (m, 3H), 0.97-0.92 (m, 3H), 0.70 (br d, J = 6.4 Hz, 3H). |
| A | 25 | 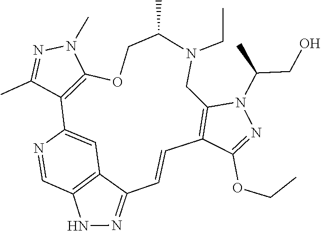 | 521.3 | ¹H NMR (400 MHz, MeOD-d₄) δ = 8.95- 8.88 (m, 1H), 8.51-8.46 (m, 1H), 7.64-7.54 (m, 1H), 7.29 (d, J = 16.4 Hz, 1H), 4.63-4.56 (m, 1H), 4.55-4.50 (m, 1H), 4.42-4.34 (m, 2H), 4.05- 3.98 (m, 1H), 3.97- 3.82 (m, 3H), 3.73 (s, 3H), 3.72-3.67 (m, 1H), 2.90-2.79 (m, 1H), 2.60- 2.57 (m, 3H), 2.55- 2.46 (m, 1H), 1.52-1.46 (m, 3H), 1.43-1.39 (m, 3H), 1.38-1.34 (m, 3H), 1.24-1.23 (m, 1H), 1.16- 1.10 (m, 3H). |
| C | 26 | 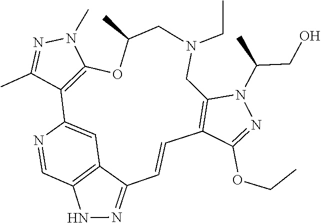 | 521.1 | ¹H NMR (400 MHz, MeOH-d₄) δ = 9.13 (d, J = 1.2 Hz, 1H), 8.66- 8.63 (m, 1H), 7.46-7.40 (m, 1H), 7.39-7.34 (m, 1H), 5.38-5.28 (m, 1H), 4.96-4.93 (m, 1H), 4.65 (d, J = 15.2 Hz, 1H), 4.53-4.47 (m, 1H), 4.46- 4.41 (m, 2H), 3.93 (dd, J = 9.2, 14.4 Hz, 1H), 3.75 (s, 3H), 3.70 (d, J = 4.8 Hz, 2H), 3.50 (d, J = 14.4 Hz, 1H), 3.42-3.35 (m, 2H), 2.54-2.53 (m, 3H), 1.54-1.48 (m, 9H), 1.23-1.20 (m, 3H). |
| B | 27 | 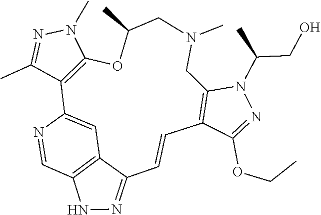 | 507.2 | ¹H NMR (400 MHz, MeOD-d₄) δ = 8.94 (s, 1H), 8.44 (s, 1H), 8.25 (d, J = 16.8 Hz, 1H), 7.25 (d, J = 16.8 Hz, 1H), 4.51-4.31 (m, 3H), 3.97 (d, J = 14.4 Hz, 1H), 3.87-3.79 (m, 2H), 3.71-3.66 (m, 4H), 3.11- 2.79 (m, 3H), 2.54 (s, 3H), 2.43 (s, 3H), 1.48 (t, J = 7.2 Hz, 3H), 1.40 (d, J = 6.8 Hz, 3H), 1.10 (d, J = 6.4 Hz, 3H). |
| C | 28 | 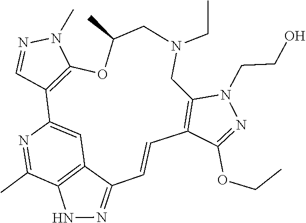 | 507.3 | ¹H NMR (400 MHz, DMSO-d₆) δ 13.41 (s, 1H), 8.27 (s, 1H), 8.08 (d, J = 17.2 Hz, 1H), 7.93 (s, 1H), 7.15 (d, J = 17.2 Hz, 1H), 4.90-4.70 (m, 2H), 4.28 (dq, J = 2.0, 7.2 Hz, 2H), 4.07- 3.89 (m, 4H), 3.68 (s, 3H), 3.66 (d, J = 5.2 Hz, 2H), 3.04-2.95 (m, 1H), 2.93-2.84 (m, 1H), 2.73 (s, 3H), 2.71-2.66 (m, 1H), 2.60 (dd, J = 7.2, 13.2 Hz, 1H), 1.42 (t, J = 7.2 Hz, 3H), 1.11 (d, J = 6.4 Hz, 3H), 0.95 (t, J = 7.2 Hz, 3H). |
| B | 29 | 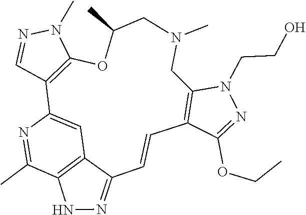 | 493.2 | ¹H NMR (400 MHz, DMSO-d₆) δ 13.33 (s, 1H), 8.23 (d, J = 17.2 Hz, 1H), 8.16 (s, 1H), 7.93 (s, 1H), 7.13 (d, J = 17.2 Hz, 1H), 4.76 (d, J = 6.4 Hz, 2H), 4.30- 4.24 (m, 2H), 4.06-3.93 (m, 4H), 3.87-3.79 (m, 1H), 3.67 (s, 3H), 3.63 (s, 1H), 2.97-2.91 (m, 1H), 2.84-2.79 (m, 1H), 2.72 (s, 3H), 2.36 (s, 3H), 1.40 (t, J = 7.2 Hz, 3H), 1.08 (d, J = 6.4 Hz, 3H). |
| D | 30 | 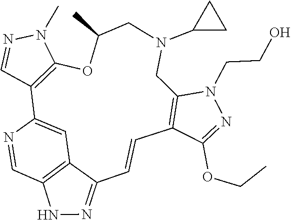 | 505.2 | ¹H NMR (400 MHz, DMSO-d₆) δ 13.30 (s, 1H), 8.93 (s, 1H), 8.27 (s, 1H), 7.95 (s, 1H), 7.52 (d, J = 16.8 Hz, 1H), 7.12 (d, J = 16.8 Hz, 1H), 4.92-4.73 (m, 2H), 4.32-4.27 (m, 2H), 4.27-4.23 (m, 1H), 4.08- 4.04 (m, 1H), 3.88 (d, J = 14.0 Hz, 1H), 3.69 (s, 3H), 3.30 (s, 3H), 3.24- 3.16 (m, 1H), 3.04-2.97 (m, 1H), 2.04 (d, J = 3.2 Hz, 1H), 1.44-1.39 (m, 3H), 1.07 (d, J = 6.0 Hz, 3H), 0.48-0.32 (m, 2H), 0.30-0.03 (m, 2H). |
| B | 31 | 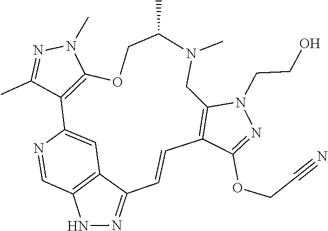 | 504.1 | ¹H NMR (400 MHz, MeOD-d₄) δ = 8.89 (s, 1H), 8.39 (s, 1H), 8.02 (br d, J = 16.8 Hz, 1H), 7.22 (d, J = 16.8 Hz, 1H), 5.15 (s, 2H), 4.27- 4.21 (m, 1H), 4.15-4.10 (m, 3H), 4.04 (br d, J = 14.8 Hz, 1H), 3.90-3.83 (m, 3H), 3.70 (s, 3H), 3.26 (br d, J = 6.0 Hz, 1H), 2.55 (s, 3H), 2.32 (s, 3H), 1.34 (d, J = 6.8 Hz, 3H). |
| A | 32 | 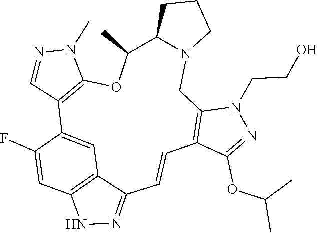 [SMILES: CC(C)Oc1nn(CCO)c2c1/C=C/c1n[nH]c3cc(F)c(cc13)-c1cnn(C)c1O[C@@H](C)[C@H]1CCCN1C2] | 519.3 | ¹H NMR (400 MHz, DMSO-d₆) δ = 13.48- 12.86 (m, 1H), 8.99- 8.88 (m, 1H), 8.85-8.72 (m, 1H), 8.36-8.17 (m, 1H), 7.30-7.09 (m, 1H), 4.49 (br d, J = 4.4 Hz, 1H), 4.34-4.24 (m, 2H), 4.13-3.94 (m, 4H), 3.69 (s, 3H), 3.65 (br d, J = 6.0 Hz, 2H), 3.21 (q, J = 7.2 Hz, 1H), 3.06 (br d, J = 4.0 Hz, 1H), 2.95- 2.85 (m, 1H), 2.52 (br s, 3H), 2.25-2.05 (m, 2H), 1.87-1.71 (m, 2H), 1.42 (t, J = 7.2 Hz, 3H), 1.33 (br d, J = 2.0 Hz, 3H), 1.26-1.15 (m, 1H). |
| A | 33 | 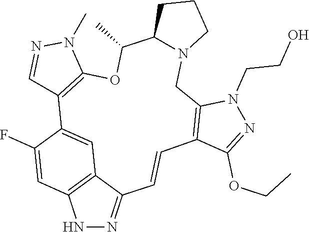 [SMILES: CCOc1nn(CCO)c2c1/C=C/c1n[nH]c3cc(F)c(cc13)-c1cnn(C)c1O[C@H](C)[C@H]1CCCN1C2] | 519.2 | ¹H NMR (400 MHz, MeOD-d₄) δ = 9.10- 8.86 (m, 1H), 8.66-8.34 (m, 1H), 8.10-7.54 (m, 1H), 7.41-7.11 (m, 1H), 4.86-4.82 (m, 1H), 4.43- 4.33 (m, 2H), 4.32- 4.21 (m, 2H), 4.20-4.09 (m, 2H), 3.99-3.84 (m, 3H), 3.73 (s, 3H), 3.68 (s, 1H), 3.35 (s, 1H), 2.53 (s, 3H), 2.35-2.10 (m, 2H), 2.09-1.74 (m, 2H), 1.50 (t, J = 7.2 Hz, 3H), 1.16-0.98 (m, 3H). |
| C | 34 | 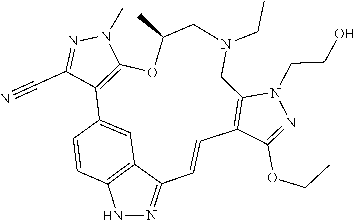 | 493.1 | ¹H NMR (400 MHz, MeOD-d₄) δ = 9.08 (s, 1H), 8.74 (s, 1H), 8.09 (s, 1H), 7.70 (br d, J = 16.4 Hz, 1H), 7.41 (d, J = 16.4 Hz, 1H), 5.33 (br s, 1H), 4.74 (br d, J = 11.6 Hz, 1H), 4.55-4.46 (m, 1H), 4.45-4.32 (m, 2H), 4.27-4.16 (m, 2H), 4.00-3.93 (m, 1H), 3.93- 3.83 (m, 2H), 3.82 (s, 3H), 3.39 (br d, J = 14.4 Hz, 2H), 3.23 (br s, 1H), 1.51 (t, J = 7.2 Hz, 3H), 1.44-1.33 (m, 3H), 1.26 (d, J = 6.4 Hz, 3H). |
| B | 35 | 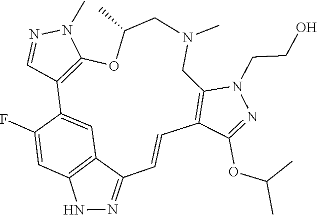 | 507.3 | ¹H NMR (400 MHz, MeOD-d₄) δ = 8.95 (s, 1H), 8.46 (s, 1H), 8.31 (d, J = 16.8 Hz, 1H), 7.26 (d, J = 16.8 Hz, 1H), 4.81 (br d, J = 5.6 Hz, 1H), 4.51-4.41 (m, 1H), 4.40-4.31 (m, 2H), 3.96-3.87 (m, 3H), 3.76- 3.66 (m, 4H), 2.99- 2.82 (m, 2H), 2.54 (s, 3H), 2.44 (s, 3H), 1.48 (t, J = 7.2 Hz, 3H), 1.35 (d, J = 6.4 Hz, 3H), 1.12 (d, J = 6.4 Hz, 3H). |
| C | 36 | 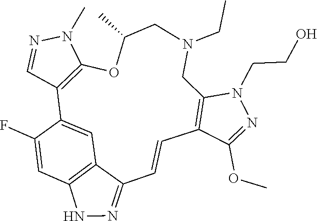 | 518.2 | ¹H NMR (400 MHz, MeOD-d₄) δ = 8.95 (s, 1H), 8.53 (s, 1H), 8.20 (d, J = 17.2 Hz, 1H), 7.18 (d, J = 17.2 Hz, 1H), 5.17 (s, 2H), 4.79- 4.68 (m, 1H), 4.24-4.11 (m, 2H), 4.09-3.95 (m, 2H), 3.88 (t, J = 5.2 Hz, 2H), 3.70 (s, 3H), 3.10- 3.01 (m, 1H), 2.96-2.86 (m, 1H), 2.79-2.62 (m, 2H), 2.55 (s, 3H), 1.11 (d, J = 6.4 Hz, 3H), 0.98 (t, J = 7.2 Hz, 3H). |
| C | 37 | 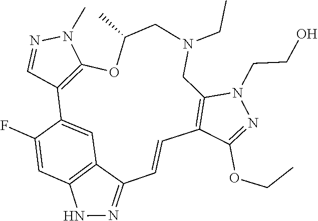 | 507.1 | ¹H NMR (400 MHz, MeOD-d₄) δ = 8.39 (s, 1H), 8.20 (d, J = 16.8 Hz, 1H), 8.07 (s, 1H), 7.30 (d, J = 16.8 Hz, 1H), 4.90-4.88 (m, 1H), 4.39-4.30 (m, 2H), 4.21- 4.06 (m, 2H), 4.05- 3.94 (m, 2H), 3.84 (t, J = 5.6 Hz, 2H), 3.75 (s, 3H), 3.17-3.05 (m, 1H), 2.91 (d, J = 14.8 Hz, 1H), 2.84 (d, J = 6.8 Hz, 1H), 2.80 (s, 3H), 2.76- 2.64 (m, 1H), 1.50 (t, J = 7.2 Hz, 3H), 1.17 (d, J = 6.4 Hz, 3H), 1.03 (t, J = 7.2 Hz, 3H). |
| B | 38 | 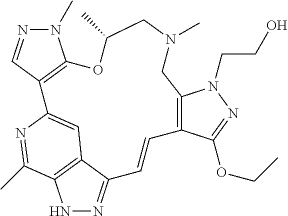 [SMILES: CCOc1nn(CCO)c2c1/C=C/c1n[nH]c3c(C)nc(cc13)-c1cnn(C)c1O[C@H](C)CN(C)C2] | 493.1 | ¹H NMR (400 MHz, MeOD-d₄) δ = 8.35 (br d, J = 16.8 Hz, 1H), 8.28 (s, 1H), 8.06 (s, 1H), 7.28 (d, J = 17.2 Hz, 1H), 4.63-4.62 (m, 1H), 4.34 (dq, J = 2.8, 7.2 Hz, 2H), 4.11 (td, J = 5.6, 8.0 Hz, 2H), 4.01-3.95 (m, 1H), 3.91-3.80 (m, 3H), 3.74 (s, 3H), 2.98- 2.89 (m, 2H), 2.79 (s, 3H), 2.46 (s, 3H), 1.54- 1.45 (m, 3H), 1.16 (d, J = 6.4 Hz, 3H). |
| C | 39 | 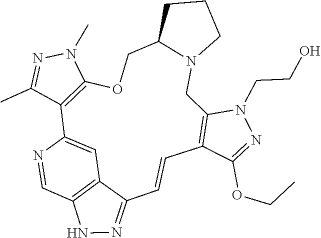 | 505.2 | ¹H NMR (400 MHz, DMSO-d₆) δ = 13.25 (br s, 1H), 8.96 (s, 1H), 8.49 (s, 1H), 7.90 (br d, J = 17.2 Hz, 1H), 7.14 (br d, J = 16.8 Hz, 1H), 4.85 (br s, 1H), 4.34-4.09 (m, 6H), 4.04 (br d, J = 4.0 Hz, 2H), 3.83 (br d, J = 14.0 Hz, 1H), 3.69 (s, 3H), 3.67-3.63 (m, 1H), 3.20 (br s, 1H), 2.76 (br t, J = 6.0 Hz, 1H), 2.52-2.50 (m, 3H), 2.41-2.33 (m, 1H), 2.09- 1.97 (m, 1H), 1.93- 1.81 (m, 1H), 1.76-1.58 (m, 2H), 1.45-1.39 (m, 3H). |
| B | 40 | 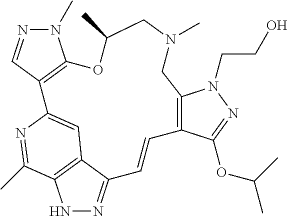 [SMILES: Cc1nc2cc3c(n[nH]c13)/C=C/c1c(OC(C)C)nn(CCO)c1CN(C)C[C@H](C)Oc1c-2cnn1C] | 507.2 | ¹H NMR (400 MHz, MeOD-d₄) δ = 8.35 (d, J = 17.2 Hz, 1H), 8.31 (s, 1H), 8.07 (s, 1H), 7.31 (d, J = 17.2 Hz, 1H), 5.02-4.93 (m, 2H), 4.16- 4.07 (m, 2H), 4.03- 3.97 (m, 1H), 3.92-3.86 (m, 1H), 3.85-3.80 (m, 2H), 3.75 (s, 3H), 2.99- 2.88 (m, 2H), 2.80 (s, 3H), 2.47 (s, 3H), 1.45 (t, J = 6.4 Hz, 6H), 1.16 (d, J = 6.4 Hz, 3H). |
| B | 41 | 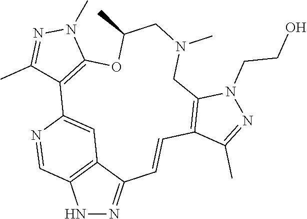 [SMILES: Cc1nn(CCO)c2c1/C=C/c1n[nH]c3cnc(cc13)-c1c(C)nn(C)c1O[C@@H](C)CN(C)C2] | 463.1 | ¹H NMR (400 MHz, DMSO-d₆) δ = 13.33 (s, 1H), 9.01-8.93 (m, 1H), 8.38 (s, 1H), 8.33 (d, J = 17.2 Hz, 1H), 6.91 (d, J = 17.6 Hz, 1H), 4.87 (t, J = 5.6 Hz, 1H), 4.75- 4.64 (m, 1H), 4.18-4.05 (m, 2H), 3.98-3.83 (m, 2H), 3.67 (td, J = 5.6, 10.8 Hz, 2H), 3.62 (s, 3H), 2.97-2.90 (m, 1H), 2.84-2.76 (m, 1H), 2.48 (s, 3H), 2.40 (s, 3H), 2.29 (s, 3H), 1.03 (d, J = 6.4 Hz, 3H). |
| B | 42 | 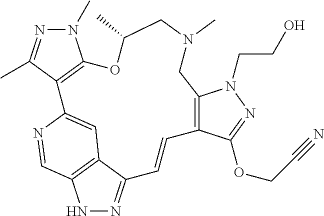 [SMILES: Cc1nn(C)c2c1-c1cc3c(n[nH]c3cn1)/C=C/c1c(OCC#N)nn(CCO)c1CN(C)C[C@@H](C)O2] | 504.2 | ¹H NMR (400 MHz, MeOD-d₄) δ = 8.95 (s, 1H), 8.44 (s, 1H), 8.32 (d, J = 17.2 Hz, 1H), 7.18 (d, J = 17.2 Hz, 1H), 5.16 (s, 2H), 4.83 (br s, 1H), 4.21-4.11 (m, 2H), 4.01-3.92 (m, 2H), 3.91-3.84 (m, 2H), 3.69 (s, 3H), 2.99-2.82 (m, 2H), 2.54 (s, 3H), 2.44 (s, 3H), 1.11 (d, J = 6.4 Hz, 3H). |
| C | 43 | 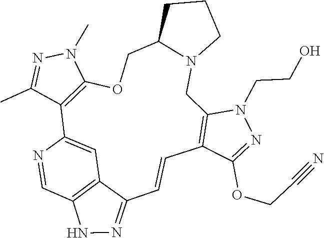 | 516.1 | ¹H NMR (400 MHz, MeOD-d₄) δ = 8.95 (s, 1H), 8.58 (s, 1H), 8.07- 7.99 (m, 1H), 7.19 (d, J = 16.8 Hz, 1H), 5.18 (s, 2H), 4.50-4.36 (m, 1H), 4.34-4.05 (m, 5H), 4.00- 3.86 (m, 3H), 3.76 (s, 3H), 3.02-2.89 (m, 1H), 2.57 (s, 4H), 2.23-2.09 (m, 1H), 1.90-1.69 (m, 3H). |
| B | 44 | 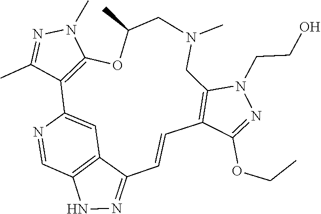 | 493.2 | ¹H NMR (400 MHz, MeOD-d₄) δ = 8.97- 8.92 (m, 1H), 8.46-8.42 (m, 1H), 8.34-8.26 (m, 1H), 7.30-7.23 (m, 1H),4.84-4.79 (m, 1H), 4.38-4.30 (m, 2H), 4.17- 4.05 (m, 2H), 3.97- 3.86 (m, 2H), 3.85-3.81 (m, 2H), 3.69-3.68 (m, 3H), 2.97-2.83 (m, 2H), 2.55-2.53 (m, 3H), 2.42 (s, 3H), 1.51-1.47 (m, 3H), 1.12-1.09 (m, 3H). |
| B | 45 | 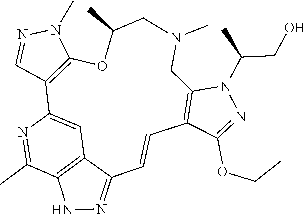 | 507.2 | ¹H NMR (400 MHz, MeOD-d₄) δ = 8.34- 8.26 (m, 2H), 8.06 (s, 1H), 7.27 (d, J = 17.2 Hz, 1H), 4.46 (d, J = 8.0, 11.6 Hz, 1H), 4.40-4.32 (m, 2H), 4.02 (d, J = 14.4 Hz, 1H), 3.87-3.79 (m, 2H), 3.75 (s, 3H), 3.70 (d, J = 4.4 Hz, 1H), 3.67 (d, J = 4.4 Hz, 1H), 2.98-2.89 (m, 2H), 2.81 (s, 3H), 2.47 (s, 3H), 1.51-1.46 (m, 3H), 1.40 (d, J = 6.8 Hz, 3H), 1.15 (d, J = 6.4 Hz, 3H). |
| B | 46 | 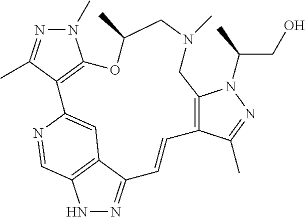 [SMILES: Cc1nn([C@@H](C)CO)c2c1/C=C/c1n[nH]c3cnc(cc13)-c1c(C)nn(C)c1O[C@@H](C)CN(C)C2] | 477.1 | ¹H NMR (400 MHz, DMSO-d₆) δ = 13.45- 13.14 (m, 1H), 8.99 (s, 1H), 8.43-8.35 (m, 2H), 6.92 (d, J = 17.6 Hz, 1H), 4.90 (br s, 1H), 4.79-4.59 (m, 1H), 4.02- 3.88 (m, 5H), 3.63 (s, 3H), 2.99-2.89 (m, 1H), 2.82 (dd, J = 7.6, 14.0 Hz, 1H), 2.49 (br s,3H), 2.41 (s, 3H), 2.29 (s, 3H), 1.14-0.96 (m, 6H). |
| B | 47 | 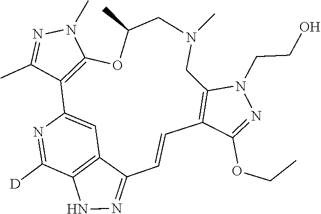 | 507.1 | ¹H NMR (400 MHz, MeOD-d₄) δ = 8.32- 8.26 (m, 1H), 8.26-8.23 (m, 1H), 7.26 (d, J = 17.2 Hz, 1H), 4.39-4.30 (m, 2H), 4.17-4.06 (m, 2H), 3.98-3.80 (m, 5H), 3.67 (s, 3H), 2.84 (s, 2H), 2.79 (s, 3H), 2.55 (s, 3H), 2.42 (s, 3H), 1.49 (t, J = 7.2 Hz, 3H), 1.10 (d, J = 6.4 Hz, 3H). |
| B | 48 | 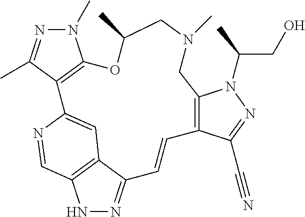 [SMILES: Cc1nn(C)c2c1-c1cc3c(n[nH]c3cn1)/C=C/c1c(C#N)nn([C@@H](C)CO)c1CN(C)C[C@H](C)O2] | 488.2 | ¹H NMR (400 MHz, MeOD-d₄) δ = 8.97 (s, 1H), 8.43-8.34 (m, 2H), 7.53 (d, J = 17.6 Hz, 1H), 4.83-4.81 (m, 1H), 4.77-4.68 (m, 2H), 4.10- 3.97 (m, 2H), 3.86- 3.74 (m, 2H), 3.68 (s, 3H), 2.97-2.86 (m, 2H), 2.53 (s, 3H), 2.39 (s, 3H), 1.47 (d, J = 6.4 Hz, 3H), 1.08 (br d, J = 6.0 Hz, 3H). |
| B | 49 | 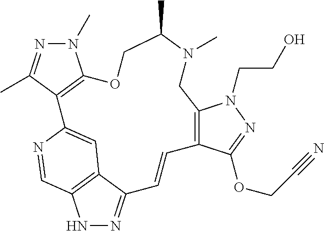 [SMILES: Cc1nn(C)c2c1-c1cc3c(n[nH]c3cn1)/C=C/c1c(OCC#N)nn(CCO)c1CN(C)[C@H](C)CO2] | 504.1 | ¹H NMR (400 MHz, MeOD-d₄) δ = 8.91 (s, 1H), 8.43 (s, 1H), 8.06 (d, J = 16.0 Hz, 1H), 7.24 (d, J = 16.0 Hz, 1H), 5.16 (s, 2H), 4.27 (dd, J = 3.6, 12.0 Hz, 1H), 4.14 (br dd, J = 3.6, 6.0 Hz, 3H), 4.06 (d, J = 12.0 Hz, 1H), 3.90-3.85 (m, 3H), 3.72 (s,3H), 3.26 (dt, J = 4.0, 8.0 Hz, 1H), 2.57 (s, 3H), 2.33 (s, 3H), 1.35 (d, J = 8.0 Hz, 3H). |
| C | 50 | 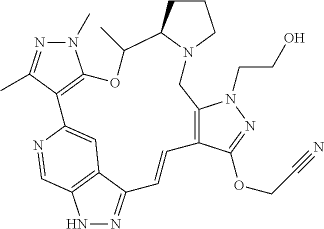 [SMILES: Cc1nn(C)c2c1-c1cc3c(n[nH]c3cn1)/C=C/c1c(OCC#N)nn(CCO)c1CN1CCC[C@@H]1C(C)O2] | 530.2 | ¹H NMR (400 MHz, MeOD-d₄) δ = 8.97- 8.93 (m, 1H), 8.55-8.47 (m, 1H), 8.02-7.95 (m, 1H), 7.21-7.14 (m, 1H), 5.18 (s, 2H), 4.37-4.25 (m, 2H), 4.21-4.11 (m, 2H), 3.98-3.87 (m, 4H), 3.75-3.73 (m, 3H), 2.92 (br s, 1H), 2.56 (s, 4H), 2.21-2.13 (m, 1H), 2.04- 2.03 (m, 3H), 1.04- 0.94 (m, 3H). |
| B | 51 | 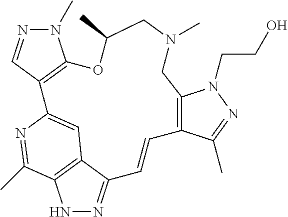 [SMILES: Cc1nn(CCO)c2c1/C=C/c1n[nH]c3c(C)nc(cc13)-c1cnn(C)c1O[C@@H](C)CN(C)C2] | 463.1 | ¹H NMR (400 MHz, MeOD-d₄) δ = 8.51 (d, J = 17.6 Hz, 1H), 8.30 (s, 1H), 8.05 (s, 1H), 7.00 (d, J = 17.6 Hz, 1H), 4.94-4.89 (m, 2H), 4.28- 4.15 (m, 2H), 4.05- 3.99 (m, 1H), 3.95-3.89 (m, 1H), 3.88-3.83 (m, 2H), 3.75 (s, 3H), 2.95- 2.92 (m, 1H), 2.82-2.79 (m, 3H), 2.50 (s, 3H), 2.42 (s, 3H), 1.14 (d, J = 6.4 Hz, 3H). |
| B | 52 | 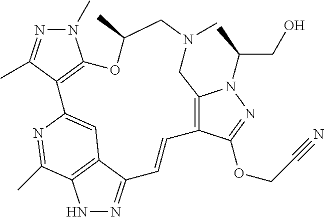 [SMILES: Cc1nn(C)c2c1-c1cc3c(n[nH]c3c(C)n1)/C=C/c1c(OCC#N)nn([C@@H](C)CO)c1CN(C)C[C@H](C)O2] | 532.2 | ¹H NMR (400 MHz, MeOD-d₄) δ = 8.31- 8.18 (m, 2H), 7.16 (d, J = 17.2 Hz, 1H), 5.16 (s, 2H), 4.93-4.89 (m, 1H), 4.58-4.42 (m, 1H), 4.02- 3.96 (m, 1H), 3.87 (d, J = 14.0 Hz, 1H), 3.84- 3.78 (m, 1H), 3.73-3.68 (m, 1H), 3.67 (s, 3H), 2.97-2.83 (m, 2H), 2.79 (s, 3H), 2.55 (s, 3H), 2.44 (s, 3H), 1.43 (d, J = 6.8 Hz, 3H), 1.09 (d, J = 6.4 Hz, 3H). |
| B | 53 | 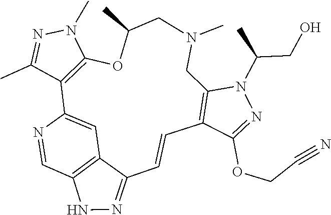 | 518.1 | ¹H NMR (400 MHz, MeOD-d₄) δ = 8.97- 8.90 (m, 1H), 8.44-8.38 (m, 1H), 8.29-8.20 (m, 1H), 7.21-7.12 (m, 1H), 5.17 (s, 2H), 4.97 (br s, 1H), 4.55-4.45 (m, 1H), 4.03-3.95 (m, 1H), 3.91-3.85 (m, 1H), 3.84-3.80 (m, 1H), 3.75- 3.71 (m, 1H), 3.70- 3.67 (m, 3H), 2.97-2.82 (m, 2H), 2.57-2.51 (m, 3H), 2.43 (s, 3H), 1.43 (d, J = 6.4 Hz, 3H), 1.09 (d, J = 6.4 Hz, 3H). |
| B | 54 | 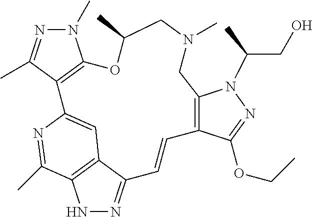 [SMILES: CCOc1nn([C@@H](C)CO)c2c1/C=C/c1n[nH]c3c(C)nc(cc13)-c1c(C)nn(C)c1O[C@@H](C)CN(C)C2] | 521.2 | ¹H NMR (400 MHz, MeOD-d₄) δ = 8.28- 8.19 (m, 2H), 7.24 (d, J = 16.8 Hz, 1H), 4.93- 4.89 (m, 1H), 4.52-4.42 (m, 1H), 4.41-4.31 (m, 2H), 4.01-3.93 (m, 1H), 3.86-3.78 (m, 2H), 3.71- 3.67 (m, 4H), 2.97- 2.82 (m, 2H), 2.79 (s, 3H), 2.56 (s, 3H), 2.43 (s, 3H), 1.48 (t, J = 7.0 Hz, 3H), 1.40 (d, J = 6.8 Hz, 3H), 1.10 (d, J = 6.4 Hz, 3H). |
| C | 55 | 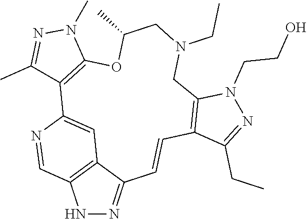 [SMILES: CCc1nn(CCO)c2c1/C=C/c1n[nH]c3cnc(cc13)-c1c(C)nn(C)c1O[C@H](C)CN(CC)C2] | 491.1 | ¹H NMR (400 MHz, MeOD-d₄) δ = 8.96 (s, 1H), 8.53-8.47 (m, 1H), 8.36-8.25 (m, 1H), 7.03- 6.94 (m, 1H), 4.78- 4.68 (m, 1H), 4.29-4.17 (m, 2H), 4.13-3.96 (m, 2H), 3.89-3.83 (m, 2H), 3.69 (s, 3H), 3.09-2.84 (m, 3H), 2.95-2.81 (m, 1H), 2.68-2.56 (m, 2H), 2.54 (s, 3H), 1.37 (t, J = 7.6 Hz, 3H), 1.07 (d, J = 6.4 Hz, 3H), 0.94 (t, J = 7.2 Hz, 3H). |
| B | 56 | 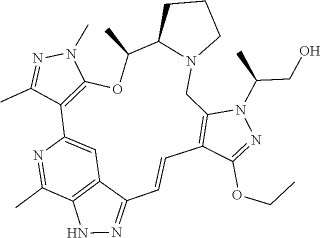 | 533.2 | ¹H NMR (400 MHz, MeOD-d₄) δ = 8.93 (s, 1H), 8.63-8.42 (m, 1H), 7.95-7.77 (m, 1H), 7.22 (d, J = 16.4 Hz, 1H), 4.95 (br d, J = 2.0 Hz, 1H), 4.69-4.56 (m, 1H), 4.42-4.17 (m, 3H), 3.98- 3.60 (m, 7H), 3.57- 3.37 (m, 1H), 3.09-2.77 (m, 1H), 2.54 (s, 3H), 2.42 (s, 1H), 2.24-2.09 (m, 1H), 1.94-1.65 (m, 2H), 1.54-1.42 (m, 6H), 1.07-0.87 (m, 3H). |
| B | 57 | 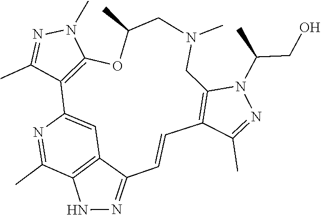 | 491.2 | ¹H NMR (400 MHz, MeOD-d₄) δ = 8.46 (d, J = 17.6 Hz, 1H), 8.24- 8.20 (m, 1H), 6.97 (d, J = 17.6 Hz, 1H), 4.85- 4.79 (m, 1H), 4.68-4.68 (m, 1H), 4.16-4.07 (m, 1H), 4.04 (d, J = 5.6 Hz, 2H), 3.97-3.90 (m, 2H), 3.67 (s, 3H), 2.87 (br d, J = 4.4 Hz, 2H), 2.79 (s, 3H), 2.54 (s, 3H), 2.49 (s, 3H), 2.36 (s, 3H), 1.18 (d, J = 6.0 Hz, 3H), 1.08 (d, J = 6.4 Hz, 3H). |
| C | 58 | 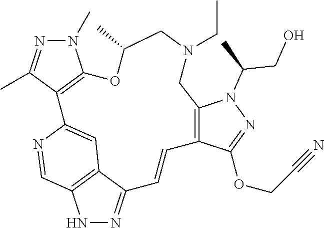 [SMILES: CCN1Cc2c(c(OCC#N)nn2[C@@H](C)CO)/C=C/c2n[nH]c3cnc(cc23)-c2c(C)nn(C)c2O[C@H](C)C1] | 532.2 | ¹H NMR (400 MHz, MeOD-d₄) δ = 8.96 (s, 1H), 8.56 (s, 1H), 8.26- 8.19 (m, 1H), 7.23-7.15 (m, 1H), 5.17 (d, J = 2.0 Hz, 2H), 4.79-4.68 (m, 1H), 4.52 (br dd, J = 6.4, 13.2 Hz, 1H), 4.07 (br s, 1H), 4.07-4.01 (m, 1H), 3.98-3.91 (m, 1H), 3.75 (dd, J = 4.8, 11.2 Hz, 1H), 3.70 (s, 3H), 3.07- 2.91 (m, 2H), 2.78-2.65 (m, 2H), 2.56 (s, 3H), 1.38 (d, J = 6.4 Hz, 3H), 1.14 (d, J = 6.4 Hz, 3H), 1.05-0.96 (m, 3H). |
| B | 59 | 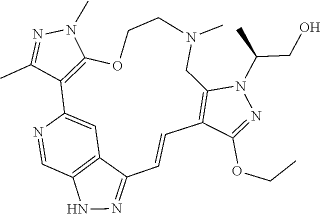 | 493.1 | ¹H NMR (400 MHz, MeOH-d₄) δ = 8.95- 8.86 (m, 1H), 8.59-8.51 (m, 1H), 8.22-8.12 (m, 1H), 7.37-7.27 (m, 1H), 4.49-4.41 (m, 1H), 4.40- 4.32 (m, 2H), 4.30- 4.17 (m, 2H), 3.96-3.78 (m, 3H), 3.73-3.66 (m, 4H), 3.04-2.99 (m, 2H), 2.60-2.56 (m, 3H), 2.31- 2.28 (m, 3H), 1.51- 1.47 (m, 3H), 1.38 (d, J = 6.8 Hz, 3H). |
| B | 60 | 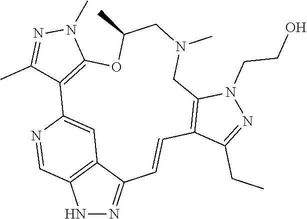 [SMILES: CCc1nn(CCO)c2c1/C=C/c1n[nH]c3cnc(cc13)-c1c(C)nn(C)c1O[C@@H](C)CN(C)C2] | 477.1 | ¹H NMR (400 MHz, MeOD-d₄) δ = 8.99- 8.94 (m, 1H), 8.49-8.44 (m, 1H), 8.43 (s, 1H), 7.03-6.99 (m, 1H), 6.98 (d, J = 17.6 Hz, 1H), 4.79 (s, 1H), 4.29-4.17 (m, 2H), 4.01 (s, 2H), 3.81 (s, 2H), 3.69 (s, 3H), 2.96-2.84 (m, 4H), 2.53 (s, 3H), 2.38 (s, 2H), 2.41-2.34 (m, 1H), 1.37 (t, J = 7.6 Hz, 3H), 1.09 (d, J = 6.4 Hz, 3H). |
| B | 61 | 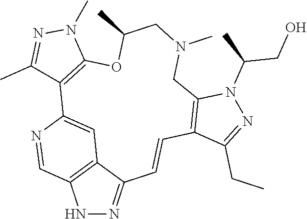 [SMILES: CCc1nn([C@@H](C)CO)c2c1/C=C/c1n[nH]c3cnc(cc13)-c1c(C)nn(C)c1O[C@@H](C)CN(C)C2] | 491.2 | ¹H NMR (400 MHz, MeOD-d₄) δ = 8.94 (s, 1H), 8.41 (s, 1H), 8.37 (d, J = 16.0 Hz, 1H), 6.96 (d, J = 16.0 Hz, 1H), 4.85-4.79 (m, 1H), 4.66-4.49 (m, 1H), 4.01- 3.95 (m, 1H), 3.88- 3.82 (m, 1H), 3.82-3.76 (m, 1H), 3.76-3.70 (m, 1H), 3.66 (s, 3H), 3.02- 2.78 (m, 4H), 2.50 (s, 3H), 2.36 (s, 3H), 1.43 (d, J = 8.0 Hz, 3H), 1.34 (t, J = 8.0 Hz, 3H), 1.05 (d, J = 6.0 Hz, 3H). |
| C | 62 | 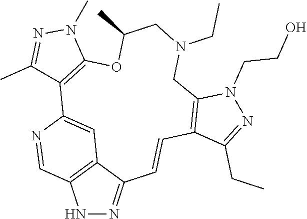 [SMILES: CCc1nn(CCO)c2c1/C=C/c1n[nH]c3cnc(cc13)-c1c(C)nn(C)c1O[C@@H](C)CN(CC)C2] | 491.1 | ¹H NMR (400 MHz, MeOD-d₄) δ = 9.00- 8.92 (m, 1H), 8.50 (s, 1H), 8.37-8.26 (m, 1H), 7.04-6.94 (m, 1H), 4.79- 4.67 (m, 1H), 4.31- 4.18 (m, 2H), 4.12-3.98 (m, 2H), 3.92-3.81 (m, 2H), 3.69 (s, 3H), 3.08- 2.98 (m, 1H), 2.96-2.83 (m, 3H), 2.65-2.56 (m, 2H), 2.54 (s, 3H), 1.41- 1.34 (m, 3H), 1.07 (d, J = 6.4 Hz, 3H), 0.94 (t, J = 7.2 Hz, 3H). |
| B | 63 | 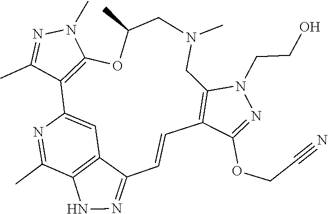 | 518.1 | ¹H NMR (400 MHz, MeOD-d₄) δ = 8.31 (d, J = 17.2 Hz, 1H), 8.25 (s, 1H), 7.17 (d, J = 17.2 Hz, 1H), 5.16 (s, 2H), 4.88-4.87 (m, 1H), 4.20- 4.11 (m, 2H), 4.01- 3.91 (m, 2H), 3.90-3.84 (m, 2H), 3.68 (s, 3H), 2.97-2.86 (m, 2H), 2.80 (s, 3H), 2.56 (s, 3H), 2.44 (s, 3H), 1.11 (d, J = 6.4 Hz, 3H). |
| C | 64 | 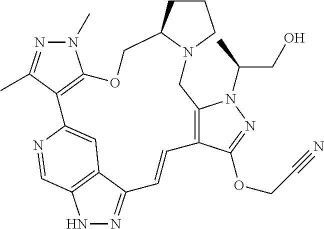 [SMILES: Cc1nn(C)c2c1-c1cc3c(n[nH]c3cn1)/C=C/c1c(OCC#N)nn([C@@H](C)CO)c1CN1CCC[C@@H]1CO2] | 530.1 | ¹H NMR (400 MHz, MeOD-d4) δ = 8.97- 8.91 (m, 1H), 8.61-8.55 (m, 1H), 8.05-7.96 (m, 1H), 7.20-7.12 (m, 1H), 5.21-5.14 (m, 2H), 4.66- 4.61 (m, 1H), 4.44- 4.34 (m, 1H), 4.21-4.03 (m, 2H), 3.88-3.78 (m, 3H), 3.76 (s, 3H), 3.74- 3.69 (m, 1H), 3.28-3.19 (m, 1H), 2.94-2.86 (m, 1H), 2.59-2.53 (m, 3H), 2.38 (dt, J = 6.8, 9.2 Hz, 1H), 2.20-2.08 (m, 1H), 2.20-2.07 (m, 1H), 1.86- 1.75 (m, 2H), 1.46 (d, J = 6.8 Hz, 3H). |
| C | 65 | 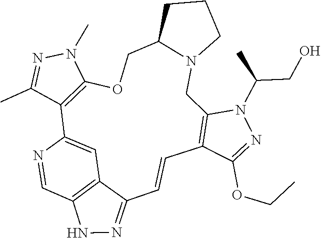 | 519.1 | ¹H NMR (400 MHz, MeOD-d₄) δ = 8.97- 8.89 (m, 1H), 8.60-8.53 (m, 1H), 7.98 (dd, J = 3.6, 16.8 Hz, 1H), 7.27- 7.18 (m, 1H), 4.60-4.53 (m, 1H), 4.42-4.31 (m, 3H), 4.18-4.03 (m, 2H), 3.86-3.76 (m, 3H), 3.75 (s, 3H), 3.72-3.65 (m, 1H), 3.27-3.19 (m, 1H), 2.95-2.85 (m, 1H), 2.56 (s, 3H), 2.42-2.32 (m, 1H), 2.42-2.32 (m, 1H), 2.19-2.06 (m, 1H), 2.17- 2.05 (m, 1H), 1.75 (br s, 2H), 1.49 (t, J = 7.2 Hz, 3H), 1.42 (d, J = 6.8 Hz, 3H). |
| B | 66 | 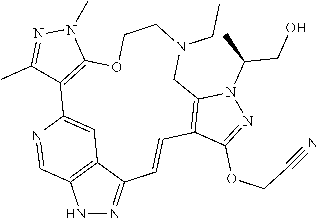 [SMILES: CCN1CCOc2c(c(C)nn2C)-c2cc3c(n[nH]c3cn2)/C=C/c2c(OCC#N)nn([C@@H](C)CO)c2C1] | 518.2 | ¹H NMR (400 MHz, MeOD-d₄) δ = 8.94 (s, 1H), 8.62 (s, 1H), 8.12 (d, J = 17.2 Hz, 1H), 7.23 (d, J = 17.2 Hz, 1H), 5.17 (s, 2H), 4.57- 4.49 (m, 1H), 4.36-4.30 (m, 1H), 4.27-4.20 (m, 1H), 4.03-3.92 (m, 2H), 3.90-3.84 (m, 1H), 3.76- 3.74 (m, 3H), 3.74- 3.70 (m, 1H), 3.13 (d, J = 4.8 Hz, 1H), 3.10- 3.00 (m, 1H), 2.65-2.53 (m, 5H), 1.42 (d, J = 6.4 Hz, 3H), 1.01 (t, J = 7.2 Hz, 3H). |
| C | 67 | 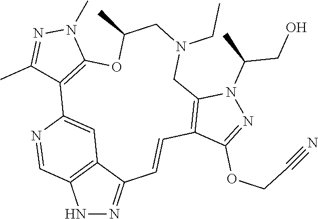 [SMILES: CCN1Cc2c(c(OCC#N)nn2[C@@H](C)CO)/C=C/c2n[nH]c3cnc(cc23)-c2c(C)nn(C)c2O[C@@H](C)C1] | 532.1 | ¹H NMR (400 MHz, DMSO-d₆) δ = 13.44- 13.25 (m, 1H), 9.00- 8.94 (m, 1H), 8.55-8.44 (m, 1H), 8.01 (d, J = 17.2 Hz, 1H), 7.20-6.97 (m, 1H), 5.26-5.20 (m, 2H), 4.88-4.81 (m, 1H), 4.75-4.67 (m, 1H), 4.52- 4.42 (m, 1H), 4.02- 3.90 (m, 2H), 3.63 (s, 3H), 3.56 (br t, J = 6.0 Hz, 2H), 3.03-2.94 (m, 1H), 2.92-2.83 (m, 1H), 2.70-2.60 (m, 2H), 2.52- 2.51 (m, 3H), 1.35 (d, J = 6.4 Hz, 3H), 1.04 (br d, J = 6.4 Hz, 3H), 0.95 (t, J = 7.2 Hz, 3H). |
| A | 68 | 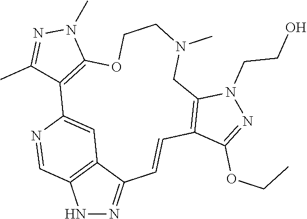 [SMILES: CCOc1nn(CCO)c2c1/C=C/c1n[nH]c3cnc(cc13)-c1c(C)nn(C)c1OCCN(C)C2] | 479.3 | ¹H NMR (499 MHz, MeOD-d₄) δ ppm 8.96 (d, J = 1.09 Hz, 1 H), 8.62 (d, J = 1.10 Hz, 1 H), 8.18 (d, J = 16.70 Hz, 2 H), 7.37 (d, J = 16.70 Hz, 1H), 4.34-4.38 (m, 4 H), 4.14 (t, J = 5.48 Hz, 2 H), 4.06 (s, 2 H), 3.85 (t, J = 5.34 Hz, 2 H), 3.75 (s, 3 H), 3.17-3.20 (m, 2 H), 2.60 (s, 3 H), 2.42 (s, 3H), 1.50 (t, J = 7.12 Hz, 3 H). |
| A | 69 | 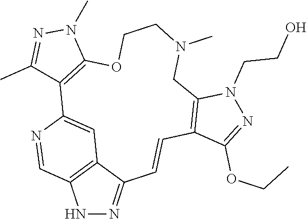 [SMILES: CCOc1nn(CCO)c2c1/C=C/c1n[nH]c3cnc(cc13)-c1c(C)nn(C)c1OCCN(C)C2] | 479.3 | ¹H NMR (400 MHz, DMSO-d6) δ ppm 9.05 (s, 1 H), 8.80 (s, 1 H), 8.10 (d, J = 16.92 Hz, 1 H), 7.21 (d, J = 17.15 Hz, 1 H), 4.86 (br t, J = 5.37 Hz, 1 H), 4.28 (q, J = 7.09 Hz, 2 H), 4.17-4.22 (m, 2 H), 4.14 (s, 3 H), 4.03 (br t, J = 5.49 Hz, 2 H), 3.78 (s, 2 H), 3.65 (q, J = 5.34 Hz, 2 H), 2.85- 2.92 (m, 2 H), 2.26 (s, 3 H), 2.12 (s, 3 H), 1.42 (t, J = 7.09 Hz, 3 H). |
| B | 70 | 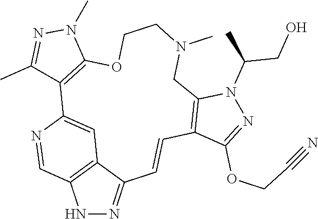 [SMILES: Cc1nn(C)c2c1-c1cc3c(n[nH]c3cn1)/C=C/c1c(OCC#N)nn([C@@H](C)CO)c1CN(C)CCO2] | 504.2 | ¹H NMR (400 MHz, MeOD-d₄) δ = 8.92 (s, 1H), 8.56 (s, 1H), 8.21 (d, J = 17.2 Hz, 1H), 7.23 (d, J = 17.2 Hz, 1H), 5.17 (s, 2H), 4.54- 4.43 (m, 1H), 4.32-4.20 (m, 2H), 3.90 (s, 1H), 3.87-3.81 (m, 1H), 3.74- 3.70 (m, 4H), 3.04 (s, 2H), 2.59 (s, 3H), 2.31 (s, 3H), 1.41 (d, J = 6.8 Hz, 3H). |
| E | 71 | 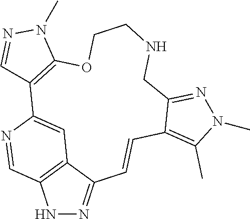 [SMILES: Cc1c2c(nn1C)CNCCOc1c(cnn1C)-c1cc3c(n[nH]c3cn1)/C=C/2] | 391.2 | ¹H NMR (400 MHz, DMSO-d6) δ 13.60- 13.37 (m, 1H), 9.47 (s, 1H), 8.99 (s, 1H), 8.48 (s, 1H), 7.97 (s, 1H), 7.58 (d, J = 17.2 Hz, 1H), 7.05 (d, J = 17.2 Hz, 1H), 4.75-4.60 (m, 2H), 4.54-4.38 (m, 2H), 3.85 (s, 3H), 3.78 (s, 3H), 3.66 (s, 2H), 2.56 (s, 3H), 1.29-1.21 (m, 1H). |
| | 72 | 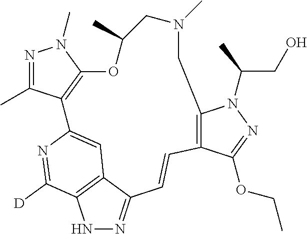 [SMILES: [2H]c1nc2cc3c(n[nH]c13)/C=C/c1c(OCC)nn([C@@H](C)CO)c1CN(C)C[C@H](C)Oc1c-2c(C)nn1C] | | |
| E | 73 | 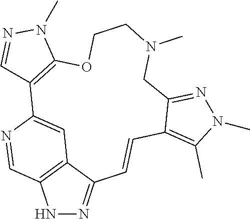 [SMILES: Cc1c2c(nn1C)CN(C)CCOc1c(cnn1C)-c1cc3c(n[nH]c3cn1)/C=C/2] | 404.8 | ¹H NMR (400 MHz, DMSO-d₆) δ ppm 13.49 (br d, J = 7.39 Hz, 1 H) 10.17 (br s, 1 H) 9.00 (s, 1 H) 8.52 (s, 1 H) 7.98 (s, 1 H) 7.60 (br d, J = 17.25 Hz, 1 H) 7.05 (br d, J = 16.97 Hz, 1 H) 5.16-5.28 (m, 1 H) 5.05- 5.15 (m, 1 H) 4.38 (br d, J = 12.87 Hz, 1 H) 3.97- 4.16 (m, 3 H) 3.86 (s, 3 H) 3.81 (s, 3 H) 2.90 (br d, J = 10.95 Hz, 3 H) 2.56 (s, 3 H). |
| B | 74 | 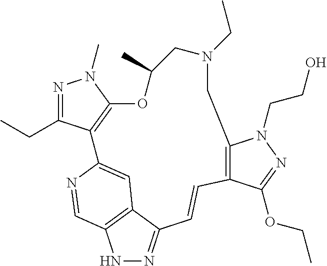 [SMILES: CCOc1nn(CCO)c2c1/C=C/c1n[nH]c3cnc(cc13)-c1c(CC)nn(C)c1O[C@@H](C)CN(CC)C2] | 521.1 | ¹H NMR (400 MHz, DMSO-d6) δ = 13.24 (s, 1H), 8.95 (s, 1H), 8.50 (s, 1H), 8.06 (d, J = 16.8 Hz, 1H), 7.14 (d, J = 17.2 Hz, 1H), 4.91- 4.78 (m, 1H), 4.72-4.57 (m, 1H), 4.32-4.24 (m, 2H), 4.04 (q, J = 5.2 Hz, 2H), 3.97-3.90 (m, 2H), 3.68-3.65 (m, 1H), 3.64 (s, 3H), 3.09-2.97 (m, 2H), 2.93 (br s, 2H), 2.57 (d, J = 7.2 Hz, 3H), 1.41 (t, J = 7.2 Hz, 3H), 1.16 (t, J = 7.2 Hz, 3H), 1.06 (d, J = 6.4 Hz, 3H), 0.92 (t, J = 7.2 Hz, 3H). |
| B | 75 | 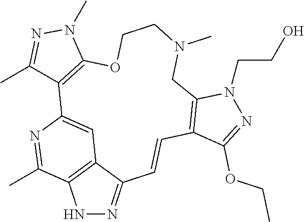 [SMILES: CCOc1nn(CCO)c2c1/C=C/c1n[nH]c3c(C)nc(cc13)-c1c(C)nn(C)c1OCCN(C)C2] | 493.2 | ¹H NMR (400 MHz, DMSO-d6) δ = 13.31 (br s, 1H), 8.34 (s, 1H), 8.10 (d, J = 17.2 Hz, 1H), 7.19 (d, J = 17.2 Hz, 1H), 4.85 (t, J = 5.6 Hz, 1H), 4.32-4.25 (m, 2H), 4.22 (br s, 2H), 4.02 (br t, J = 5.6 Hz, 2H), 3.86 (s, 2H), 3.70- 3.62 (m, 5H), 2.97 (br s, 2H), 2.73 (s, 3H), 2.54 (s, 3H), 2.19 (s, 3H), 1.42 (t, J = 7.2 Hz, 3H). |
| C | 77 | 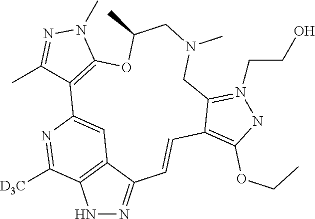 | | |
| C | 78 | 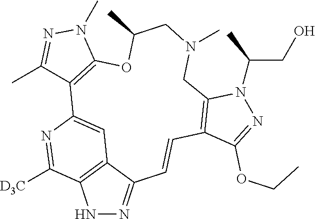 [SMILES: [2H]C([2H])([2H])c1nc2cc3c(n[nH]c13)/C=C/c1c(OCC)nn([C@@H](C)CO)c1CN(C)C[C@H](C)Oc1c-2c(C)nn1C] | | |
| C | 79 | 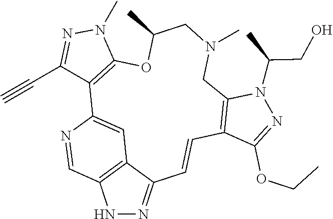 [SMILES: C#Cc1nn(C)c2c1-c1cc3c(n[nH]c3cn1)/C=C/c1c(OCC)nn([C@@H](C)CO)c1CN(C)C[C@H](C)O2] | | |
| C | 80 | 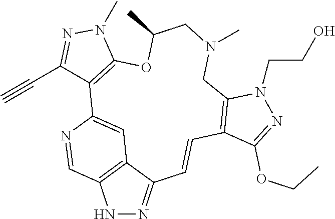 [SMILES: C#Cc1nn(C)c2c1-c1cc3c(n[nH]c3cn1)/C=C/c1c(OCC)nn(CCO)c1CN(C)C[C@H](C)O2] | | |
| B | 81 | 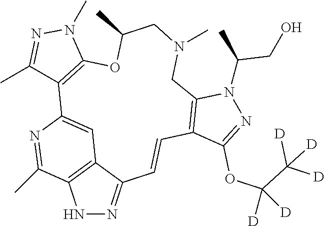 [SMILES: [2H]C([2H])([2H])C([2H])([2H])Oc1nn([C@@H](C)CO)c2c1/C=C/c1n[nH]c3c(C)nc(cc13)-c1c(C)nn(C)c1O[C@@H](C)CN(C)C2] | 526.3 | ¹H NMR (400 MHz, METHANOL-d4) δ = 8.31-8.24 (m, 1H), 8.24- 8.14 (m, 1H), 7.20 (s, 1H), 4.50-4.41 (m, 1H), 4.03-3.90 (m, 1H), 3.88- 3.75 (m, 2H), 3.70- 3.67 (m, 3H), 3.27-3.19 (m, 2H), 3.00-2.84 (m, 2H), 2.80 (s, 3H), 2.55 (s, 3H), 2.48-2.39 (m, 3H), 1.40 (s, 3H), 1.10 (d, J = 6.4 Hz, 3H). |
| C | 82 | 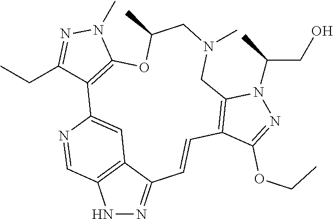 [SMILES: CCOc1nn([C@@H](C)CO)c2c1/C=C/c1n[nH]c3cnc(cc13)-c1c(CC)nn(C)c1O[C@@H](C)CN(C)C2] | 521.2 | ¹H NMR (400 MHz, DMSO-d6) δ = 13.38- 13.15 (m, 1H), 9.10- 8.85 (m, 1H), 8.51-8.34 (m, 1H), 8.12 (d, J = 17.2 Hz, 1H), 7.12 (d, J = 16.8 Hz, 1H), 4.84- 4.78 (m, 1H), 4.74-4.66 (m, 1H), 4.44-4.33 (m, 1H), 4.32-4.23 (m, 2H), 3.98-3.90 (m, 1H), 3.85- 3.77 (m, 1H), 3.63 (s, 3H), 3.57-3.52 (m, 2H), 3.09-2.99 (m, 2H), 2.88- 2.72 (m, 2H), 2.32 (s, 3H), 1.41 (t, J = 7.2 Hz, 3H), 1.31 (d, J = 6.4 Hz, 3H), 1.15 (t, J = 7.2 Hz, 3H), 1.03 (d, J = 6.4 Hz, 3H). |
| | 83 | 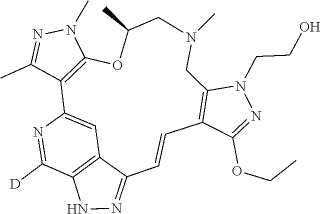 | | |
| | 84 | 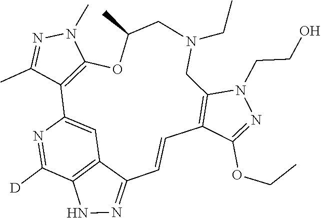 | | |
| | 85 | 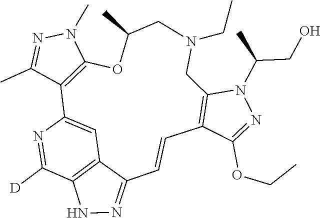 | | |
| B | 86 | 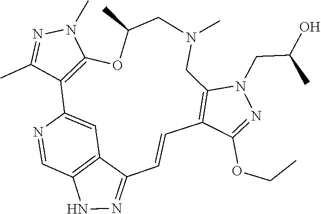 | 507.2 | ¹H NMR (400 MHz, MeOH-d₄) δ = 8.94 (s, 1H), 8.41 (s, 1H), 8.33- 8.24 (m, 1H), 7.26 (d, J = 16.8 Hz, 1H), 4.81 (br d, J = 6.4 Hz, 1H), 4.39 (br s, 2H), 4.07 (br s, 1H), 4.02-3.88 (m, 3H), 3.88-3.82 (m, 1H), 3.68 (s, 3H), 2.97-2.80 (m, 2H), 2.53 (s, 3H), 2.41 (s, 3H), 1.49 (s, 3H), 1.21 (d, J = 6.4 Hz, 3H), 1.09 (d, J = 6.4 Hz, 3H). |
| C | 87 | 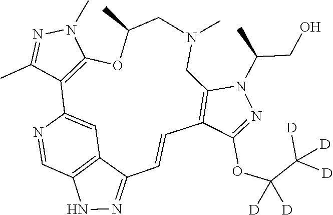 | 512.2 | ¹H NMR (400 MHz, DMSO-d₆) δ = 13.32- 13.16 (m, 1H), 9.01- 8.89 (m, 1H), 8.48-8.30 (m, 1H), 8.19-8.04 (m, 1H), 7.22-7.10 (m, 1H), 4.91-4.85 (m, 1H), 4.84- 4.73 (m, 1H), 4.73- 4.64 (m, 1H), 3.98-3.90 (m, 1H), 3.84-3.77 (m, 1H), 3.71-3.65 (m, 1H), 3.62 (s, 3H), 3.60-3.52 (m, 1H), 3.02-2.93 (m, 1H), 2.86-2.76 (m, 1H), 2.48 (br s, 3H), 2.33 (s, 3H), 1.36-1.26 (m, 3H), 1.14-0.97 (m, 3H). |
| C | 88 | 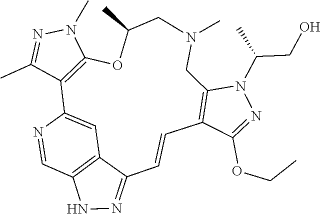 | 507.2 | ¹H NMR (400 MHz, DMSO-d₆) δ = 13.63- 12.88 (m, 1H), 8.96 (s, 1H), 8.40 (s, 1H), 8.20 (d, J = 17.2 Hz, 1H), 7.14 (d, J = 17.2 Hz, 1H), 4.90-4.74 (m, 1H), 4.68 (t, J = 6.0 Hz, 1H), 4.42-4.33 (m, 1H), 4.32- 4.24 (m, 2H), 3.96- 3.83 (m, 2H), 3.70-3.64 (m, 1H), 3.62 (s, 3H), 3.57-3.52 (m, 1H), 2.99- 2.77 (m, 2H), 2.49 (d, J = 2.0 Hz, 3H), 2.34 (s, 3H), 1.41 (t, J = 7.2 Hz, 3H), 1.28 (d, J = 6.8 Hz, 3H), 1.06 (d, J = 6.4 Hz, 3H). |
| C | 89 | 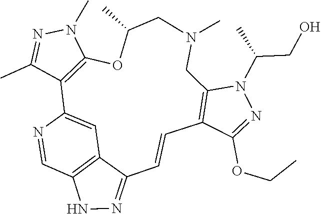 | 507.3 | ¹H NMR (400 MHz, DMSO-d₆) δ = 13.31- 13.22 (m, 1H), 8.97 (s, 1H), 8.38 (s, 1H), 8.14 (d, J = 17.2 Hz, 1H), 7.13 (d, J = 17.2 Hz, 1H), 4.82 ( t, J = 5.2 Hz, 1H), 4.76-4.68 (m, 1H), 4.45-4.35 (m, 1H), 4.34- 4.23 (m, 2H), 3.95 (d, J = 14.4 Hz, 1H), 3.81 ( d, J = 14.4 Hz, 1H), 3.62 (s, 3H), 3.57-3.52 (m, 2H), 3.01-2.93 (m, 1H), 2.81 ( dd, J = 7.6, 14.4 Hz, 1H), 2.49 ( s, 3H), 2.33 (s, 3H), 1.42 (t, J = 7.2 Hz, 3H), 1.32 (d, J = 6.8 Hz, 3H), 1.04 (d, J = 6.4 Hz, 3H). |
| C | 90 | 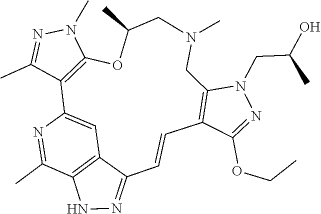 | 521.3 | ¹H NMR (400 MHz, MeOH-d₄) δ = 8.26 (br d, J = 17.2 Hz, 1H), 8.22 (s, 1H), 7.25 (d, J = 16.8 Hz, 1H), 4.39-4.28 (m, 2H), 4.11-4.02 (m, 1H), 4.02-3.88 (m, 3H), 3.87- 3.80 (m, 1H), 3.67 (s, 3H), 2.95-2.81 (m, 2H), 2.78 (s, 3H), 2.55 (s, 3H), 2.41 (s, 3H), 1.49 (s, 3H), 1.21 (d, J = 6.4 Hz, 3H), 1.09 (d, J = 6.4 Hz, 3H). |
| B | 91 | 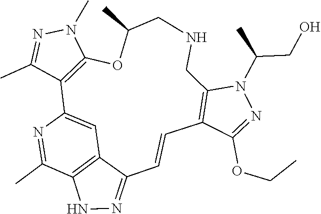 | 507.1 | ¹H NMR (400 MHz, MeOD-d₄) δ = 8.50 (d, J = 16.8 Hz, 1H), 8.31 (s, 1H), 7.19 (d, J = 16.8 Hz, 1H), 4.74 (t, J = 6.4 Hz, 1H), 4.39-4.31 (m, 3H), 4.27 (d, J = 14.4 Hz, 1H), 4.00 (d, J = 14.4 Hz, 1H), 3.80 (d, J = 8.4, 11.2 Hz, 1H), 3.71- 3.66 (m, 4H), 3.22- 3.15 (m, 1H), 2.96 (d, J = 2.4, 13.2 Hz, 1H), 2.80 (s, 3H), 2.54 (s, 3H), 1.47 (t, J = 7.2 Hz, 3H), 1.42 (d, J = 6.8 Hz, 3H), 1.16 (d, J = 6.4 Hz, 3H). |
| B | 92 | 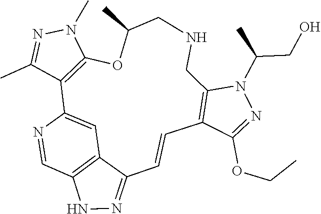 | 493.2 | ¹H NMR (400 MHz, DMSO-d₆) δ 13.19 (s, 1H), 8.95 (s, 1H), 8.50- 8.43 (m, 2H), 7.08 (d, J = 17.2 Hz, 1H), 4.90- 4.75 (m, 1H), 4.65-4.51 (m, 1H), 4.32-4.25 (m, 3H), 4.15 (d, J = 14.4 Hz, 1H), 3.90 (d, J = 14.4 Hz, 1H), 3.63 (s, 3H), 3.58-3.49 (m, 2H), 3.09-3.02 (m, 1H), 3.00- 2.93 (m, 1H), 2.49 (s, 3H), 1.41 (t, J = 7.2 Hz, 3H), 1.32 (d, J = 6.4 Hz, 3H), 1.09 (d, J = 6.4 Hz, 3H). |
| C | 93 | 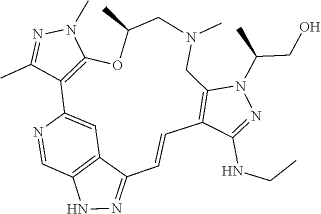 | 506.2 | ¹H NMR (400 MHz, MeOD-d₄) δ = 8.95 (s, 1H), 8.42 (s, 1H), 8.35 (d, J = 17.6 Hz, 1H), 6.91 (d, J = 17.6 Hz, 1H), 4.93 (d, J = 5.2 Hz, 1H), 4.53-4.40 (m, 1H), 4.00-3.89 (m, 1H), 3.88- 3.78 (m, 2H), 3.74- 3.66 (m, 4H), 3.39-3.34 (m, 2H), 2.94-2.79 (m, 2H), 2.52 (s, 3H), 2.41 (s, 3H), 1.43 (d, J = 6.8 Hz, 3H), 1.30 (t, J = 7.2 Hz, 3H), 1.07 (d, J = 6.3 Hz, 3H). |
| C | 94 | 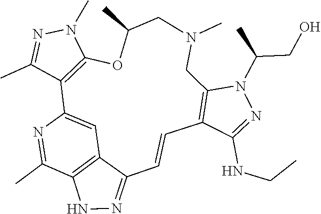 | 520.2 | ¹H NMR (400 MHz, MeOH-d₄) δ = 8.38- 8.28 (m, 1H), 8.27-8.21 (m, 1H), 6.93-6.86 (m, 1H), 4.93-4.90 (m, 1H), 4.52-4.43 (m, 1H), 3.97- 3.89 (m, 1H), 3.87- 3.78 (m, 2H), 3.67 (br s, 4H), 3.38-3.34 (m, 2H), 2.93-2.78 (m, 5H), 2.56- 2.52 (m, 3H), 2.41 (s, 3H), 1.45-1.40 (m, 3H), 1.33-1.28 (m, 3H), 1.07 (d, J = 6.4 Hz, 3H). |
| C | 95 | 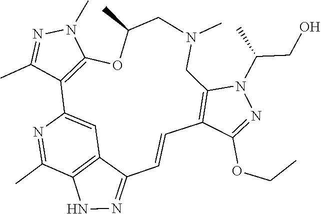 | 521.3 | ¹H NMR (400 MHz, MeOD-d₄) δ = 8.34- 8.22 (m, 2H), 7.26 (d, J = 16.8 Hz, 1H), 4.95- 4.91 (m, 1H), 4.45 (br dd, J = 6.6, 12.4 Hz, 1H), 4.40-4.31 (m, 2H), 3.99- 3.86 (m, 3H), 3.79- 3.65 (m, 4H), 2.96-2.84 (m, 2H), 2.83-2.73 (m, 3H), 2.63-2.52 (m, 3H), 2.43 (s, 3H), 1.48 (t, J = 7.2 Hz, 3H), 1.35 (d, J = 6.4 Hz, 3H), 1.11 (d, J = 6.4 Hz, 3H). |
| C | 96 | 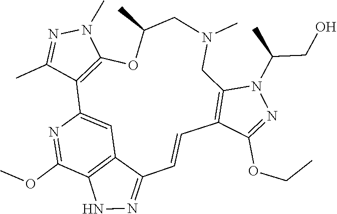 | 537.3 | ¹H NMR (400 MHz, MeOD-d₄) δ = 8.24 (br d, J = 16.4 Hz, 1H), 8.04 (s, 1H), 7.24 (br d, J = 17.2 Hz, 1H), 4.97-4.93 (m, 1H), 4.49-4.42 (m, 1H), 4.39-4.32 (m, 2H), 4.18 (s, 3H), 3.98 (br d, J = 14.4 Hz, 1H), 3.86- 3.79 (m, 2H), 3.70 (br d, J = 4.4 Hz, 1H), 3.68 (s, 3H), 2.98-2.83 (m, 2H), 2.64 (s, 3H), 2.44 (s, 3H), 1.48 (t, J = 7.2 Hz, 3H), 1.40 (d, J = 6.8 Hz, 3H), 1.12 (d, J = 6.0 Hz, 3H). |
| B | 97 | 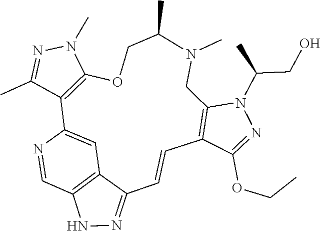 | 507.2 | ¹H NMR (400 MHz, MeOD-d₄) δ = 8.90 (s, 1H), 8.42 (s, 1H), 7.97 (d, J = 16.8 Hz, 1H), 7.33 (d, J = 16.8 Hz, 1H), 4.36 (q, J = 7.2 Hz, 3H), 4.32-4.16 (m, 2H), 4.00 (d, J = 14.4 Hz, 1H), 3.87-3.78 (m, 2H), 3.75-3.68 (m, 4H), 3.29- 3.22 (m, 1H), 2.56 (s, 3H), 2.35 (s, 3H), 1.48 (t, J = 7.2 Hz, 3H), 1.40 (d, J = 6.8 Hz, 3H), 1.34 (d, J = 6.8 Hz, 3H). |
| C | 98 | 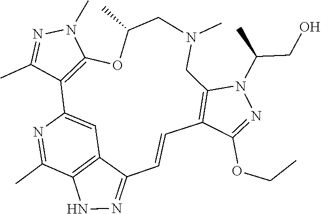 | 521.3 | ¹H NMR (400 MHz, DMSO-d₆) δ = 13.32 (s, 1H), 8.25-8.07 (m, 2H), 7.13 (d, J = 17.2 Hz, 1H), 4.92-4.76 (m, 1H), 4.74-4.61 (m, 1H), 4.41- 4.33 (m, 1H), 4.32- 4.23 (m, 2H), 3.98-3.81 (m, 2H), 3.61 (s, 3H), 3.20-3.10 (m, 2H), 2.97- 2.90 (m, 1H), 2.80 (dd, J = 7.2, 14.4 Hz, 1H), 2.73 (s, 3H), 2.34 (s, 3H), 1.41 (t, J = 7.2 Hz, 3H), 1.28 (d, J = 6.4 Hz, 3H), 1.05 (d, J = 6.0 Hz, 3H), 0.93 (t, J = 7.2 Hz, 3H). |
| C | 99 | 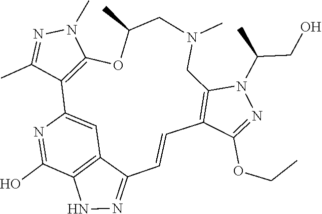 | 523.1 | ¹H NMR (400 MHz, MeOD-d₄) δ = 7.94 (br d, J = 17.2 Hz, 1H), 7.15- 7.01 (m, 2H), 5.14- 5.04 (m, 1H), 4.51-4.41 (m, 1H), 4.39-4.29 (m, 2H), 3.94-3.88 (m, 1H), 3.84-3.76 (m, 2H), 3.70- 3.64 (m, 4H), 2.93- 2.74 (m, 2H), 2.42 (s, 3H), 2.39 (s, 3H), 1.46 (t, J = 7.2 Hz, 3H), 1.39 (d, J = 6.8 Hz, 3H), 1.11 (d, J = 6.4 Hz, 3H). |
| C | 100 | 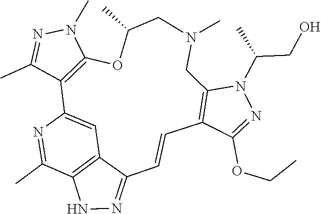 | 521.2 | ¹H NMR (400 MHz, DMSO-d₆) δ = 13.31 (s, 1H), 8.19 (s, 1H), 8.12 (d, J = 17.2 Hz, 1H), 7.12 (d, J = 17.2 Hz, 1H), 4.85-4.70 (m, 2H), 4.44-4.36 (m, 1H), 4.33- 4.25 (m, 2H), 3.98- 3.91 (m, 1H), 3.80 (d, J = 14.4 Hz, 1H), 3.61 (s, 3H), 3.56 (d, J = 9.6 Hz, 2H), 2.98-2.90 (m, 1H), 2.84-2.78 (m, 1H), 2.74 (s, 3H), 2.50-2.49 (m, 3H), 2.33 (s, 3H), 1.42 (t, J = 7.2 Hz, 3H), 1.32 (d, J = 6.4 Hz, 3H), 1.03 (d, J = 6.0 Hz, 3H). |
| C | 101 | 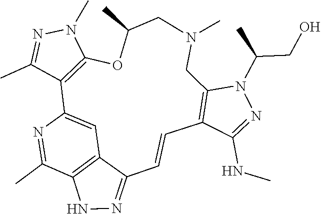 | 506.2 | ¹H NMR (400 MHz, MeOH-d₄) = 8.39-8.30 (m, 1H), 8.27-8.21 (m, 1H), 6.92-6.82 (m, 1H), 5.07 (br d, J = 2.0 Hz, 1H), 4.52-4.42 (m, 1H), 3.93 (br d, J = 14.4 Hz, 1H), 3.89-3.84 (m, 1H), 3.83-3.78 (m, 1H), 3.74- 3.70 (m, 1H), 3.68 (s, 3H), 2.94 (s, 3H), 2.89- 2.83 (m, 2H), 2.80 (s, 3H), 2.54 (s, 3H), 2.41 (s, 3H), 1.43 (d, J = 6.8 Hz, 3H), 1.07 (d, J = 6.4 Hz, 3H). |
| C | 102 | 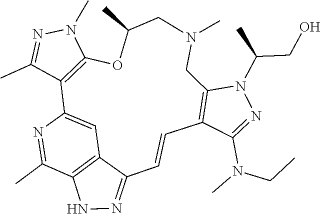 | 534.3 | ¹H NMR (400 MHz, MeOH-d₄) δ = 8.38- 8.24 (m, 2H), 7.21-7.10 (m, 1H), 4.92-4.87 (m, 1H), 4.56-4.45 (m, 1H), 3.99-3.93 (m, 1H), 3.88- 3.85 (m, 1H), 3.84- 3.81 (m, 1H), 3.74-3.69 (m, 1H), 3.69-3.67 (m, 3H), 3.23-3.16 (m, 2H), 2.89-2.85 (m, 2H), 2.84- 2.81 (m, 3H), 2.81- 2.79 (m, 3H), 2.55 (s, 3H), 2.42-2.38 (m, 3H), 1.45-1.41 (m, 3H), 1.24- 1.18 (m, 3H), 1.10- 1.05 (m, 3H). |
| C | 103 | 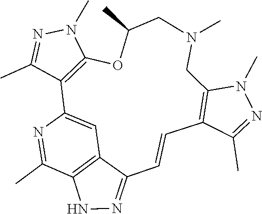 | 477.3 | ¹H NMR (400 MHz, MeOD-d₄) δ = 8.38- 8.30 (m, 1H), 8.27-8.23 (m, 1H), 7.01-6.92 (m, 1H), 4.84-4.78 (m, 1H), 3.99-3.92 (m, 1H), 3.88- 3.81 (m, 1H), 3.81- 3.77 (m, 3H), 3.71-3.65 (m, 3H), 2.93 (s, 2H), 2.82-2.78 (m, 3H), 2.57- 2.52 (m, 3H), 2.50- 2.46 (m, 3H), 2.38-2.33 (m, 3H), 1.20-0.92 (m, 3H). |
| B | 104 | 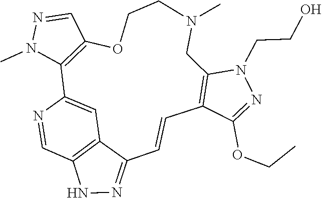 | 465.3 | ¹H NMR (500 MHz, DMSO-d6) δ ppm 9.00 (s, 1 H), 8.88 (s, 1 H), 7.88 (d, J = 17.25 Hz, 1 H), 7.45 (s, 1 H), 7.25 (d, J = 16.97 Hz, 1 H), 4.80-4.90 (m, 1 H), 4.28 (q, J = 7.12 Hz, 2 H), 4.21 (s, 3 H), 4.16-4.20 (m, 2 H), 4.04 (t, J = 5.61 Hz, 2 H), 3.72 (s, 2 H), 3.65 (br t, J = 5.61 Hz, 2 H), 2.87 (br s, 2 H), 2.11 (s, 3 H), 1.41 (t, J = 6.98 Hz, 3 H) |
| C | 105 | 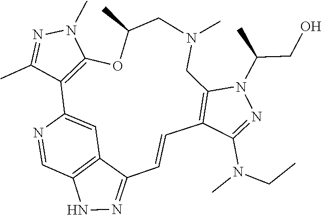 | 520.2 | ¹H NMR (400 MHz, MeOD-d₄) δ = 8.96 (s, 1H), 8.46 (s, 1H), 8.33 (d, J = 17.6 Hz, 1H), 7.17 (d, J = 16.8 Hz, 1H), 4.74-4.72 (m, 1H), 4.51-4.45 (m, 1H), 3.95 (s, 1H), 3.89-3.79 (m, 2H), 3.76-3.67 (m, 4H), 3.19 (br dd, J = 1.6, 7.2 Hz, 2H), 2.92-2.85 (m, 2H), 2.83 (s, 3H), 2.53 (s, 3H), 2.40 (s, 3H), 1.43 (d, J = 6.8 Hz, 3H), 1.21 (t, J = 6.8 Hz, 3H), 1.08 (d, J = 6.4 Hz, 3H). |
| A | 106 | 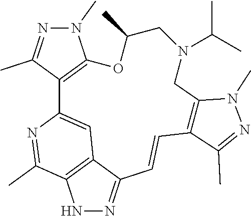 | 475.2 | ¹H NMR (400 MHz, MeOD-d₄) δ = 8.34 (s, 1H), 8.12 (d, J = 17.6 Hz, 1H), 6.99 (d, J = 17.6 Hz, 1H), 4.72-4.58 (m, 1H), 4.08-3.97 (m, 2H), 3.84 (s, 3H), 3.70 (s, 3H), 2.97-2.86 (m, 1H), 2.80 (s, 4H), 2.75- 2.67 (m, 1H), 2.56 (s, 3H), 2.48 (s, 3H), 1.06 (d, J = 6.4 Hz, 3H), 0.99 (d, J = 6.4 Hz, 3H), 0.93 (d, J = 6.4 Hz, 3H). |
| A | 107 | 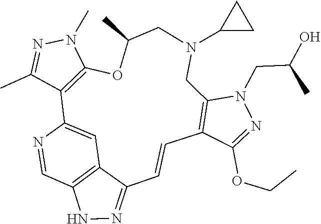 | 533.2 | ¹H NMR (400 MHz, DMSO-d₆) δ = 13.44- 13.08 (m, 1H), 9.20- 8.89 (m, 1H), 8.51-8.24 (m, 1H), 7.53-7.31 (m, 1H), 7.15 (d, J = 16.8 Hz, 1H), 4.99-4.81 (m, 1H), 4.55 (dd, J = 5.2, 8.8 Hz, 1H), 4.28 (q, J = 7.2 Hz, 2H), 4.15-4.05 (m, 2H), 3.97 (s, 2H), 3.87-3.77 (m, 2H), 3.69 (s, 3H), 3.64-3.60 (m, 1H), 2.59- 2.57 (m, 3H), 2.17- 2.03 (m, 1H), 1.40 (t, J = 7.2 Hz, 3H), 1.33 (d, J = 6.4 Hz, 3H), 1.08 (d, J = 5.6 Hz, 3H), 0.47-0.31 (m, 2H), 0.29-0.14 (m, 2H). |
| A | 108 | 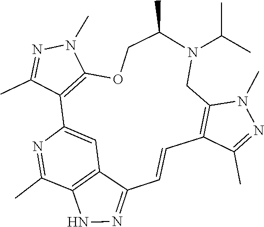 | 475.2 | ¹H NMR (400 MHz, MeOD-d₄) δ = 8.36 (s, 1H), 7.78 (d, J = 17.2 Hz, 1H), 7.01 (d, J = 17.2 Hz, 1H), 4.38 (d, J = 5.6, 8.8 Hz, 1H), 4.02- 3.94 (m, 1H), 3.92- 3.82 (m, 5H), 3.71 (s, 3H), 3.37 (d, J = 6.8 Hz, 1H), 2.96-2.86 (m, 1H), 2.78 (s, 3H), 2.60 (s, 3H), 2.48 (s, 3H), 1.39 (d, J = 7.2 Hz, 3H), 1.12 (d, J = 6.4 Hz, 3H), 0.97 (d, J = 6.4 Hz, 3H). |
| A | 109 | 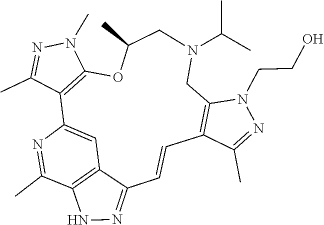 [SMILES: Cc1nn(CCO)c2c1/C=C/c1n[nH]c3c(C)nc(cc13)-c1c(C)nn(C)c1O[C@@H](C)CN(C(C)C)C2] | 505.3 | ¹H NMR (400 MHz, MeOD-d₄) δ = 8.64- 8.41 (m, 1H), 7.76-7.56 (m, 1H), 7.18 (d, J = 16.8 Hz, 1H), 5.38-5.16 (m, 1H), 4.76 (s, 1H), 4.38-4.29 (m, 1H), 4.18 (ddd, J = 3.6, 7.6, 14.4 Hz, 1H), 4.03 (d, J = 11.2 Hz, 1H), 3.88 (dt, J = 3.6, 7.6 Hz, 2H), 3.74 (s, 5H), 3.30-3.25 (m, 1H), 3.01 (s, 3H), 2.57 (s, 3H), 2.51 (s, 3H), 1.46 (d, J = 6.4 Hz, 6H), 1.18 (d, J = 6.0 Hz, 3H). |
| A | 110 | 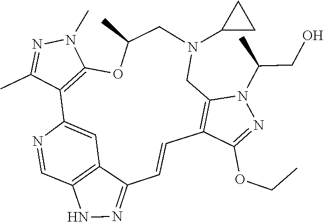 [SMILES: CCOc1nn([C@@H](C)CO)c2c1/C=C/c1n[nH]c3cnc(cc13)-c1c(C)nn(C)c1O[C@@H](C)CN(C1CC1)C2] | 533.2 | ¹H NMR (400 MHz, MeOD-d₄) δ = 8.96- 8.91 (m, 1H), 8.47-8.42 (m, 1H), 7.66-7.57 (m, 1H), 7.29-7.19 (m, 1H), 4.75-4.66 (m, 2H), 4.44-4.32 (m, 2H), 4.11- 3.97 (m, 2H), 3.82- 3.74 (m, 1H), 3.71 (s, 3H), 3.70 (s, 1H), 3.24- 3.20 (m, 1H), 3.14-3.03 (m, 1H), 2.57 (s, 2H), 2.60-2.53 (m, 1H), 2.06- 1.99 (m, 1H), 1.53- 1.43 (m, 6H), 1.05 (d, J = 6.4 Hz, 3H), 0.56- 0.14 (m, 4H). |
| A | 111 | 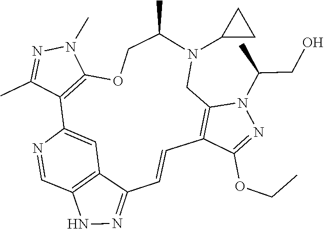 [SMILES: CCOc1nn([C@@H](C)CO)c2c1/C=C/c1n[nH]c3cnc(cc13)-c1c(C)nn(C)c1OC[C@@H](C)N(C1CC1)C2] | 533.2 | ¹H NMR (400 MHz, MeOD-d₄) δ = 8.98 (br d, J = 1.4 Hz, 1H), 8.47- 8.39 (m, 1H), 7.44 (s, 1H), 7.36-7.25 (m, 1H), 4.60-4.53 (m, 2H), 4.37 (q, J = 7.2 Hz, 2H), 4.24-4.13 (m, 2H), 3.91- 3.83 (m, 2H), 3.81- 3.73 (m, 4H), 3.60-3.51 (m, 1H), 2.56 (s, 3H), 2.21-2.08 (m, 1H), 1.51- 1.46 (m, 3H), 1.42 (s, 6H), 0.51-0.31 (m, 4H). |
| B | 112 | 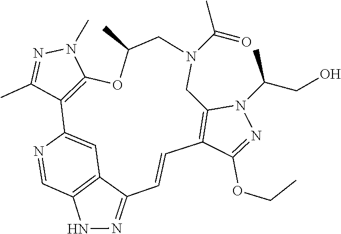 | 535.35 | ¹H NMR (400 MHz, DMSO-d6) δ ppm 8.98 (s, 1 H), 8.49 (br s, 1 H), 7.51 (br d, J = 16.47 Hz, 1 H), 7.24 (d, J = 16.24 Hz, 1 H), 5.11-5.20 (m, 1 H), 4.77 (br s, 2 H), 4.30 (td, J = 6.98, 3.20 Hz, 3 H), 3.79 (br dd, J = 15.32, 7.09 Hz, 1 H), 3.64- 3.71 (m, 3 H), 3.56- 3.63 (m, 2 H), 3.51 (br d, J = 5.49 Hz, 1 H), 2.53- 2.58 (m, 3 H), 2.16- 2.35 (m, 3 H), 1.42 (t, J = 7.09 Hz, 3 H), 1.16 (br d, J = 6.17 Hz, 3 H), 1.10 (br d, J = 4.35 Hz, 3 H). |
| A | 113 | 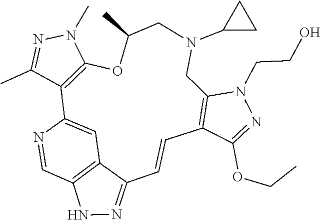 | 519.2 | ¹H NMR (400 MHz, MeOD-d₄) δ = 8.94 (d, J = 1.2 Hz, 1H), 8.42 (s, 1H), 7.59 (d, J = 16.8 Hz, 1H), 7.23 (d, J = 16.8 Hz, 1H), 4.44-4.29 (m, 4H), 4.03 (d, J = 9.6 Hz, 3H), 3.95-3.82 (m, 3H), 3.70 (s, 3H), 2.55 (s, 3H), 2.02 (td, J = 3.2, 6.4 Hz, 2H), 1.50 (t, J = 7.2 Hz, 3H), 1.28 (t, J = 7.2 Hz, 3H), 1.19-1.15 (m, 1H), 1.04 (d, J = 6.4 Hz, 3H). |
| A | 114 | 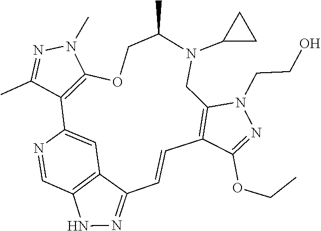 | 519.2 | ¹H NMR (400 MHz, MeOD-d₄) δ = 8.92 (s, 1H), 8.44 (s, 1H), 7.52- 7.44 (m, 1H), 7.31-7.23 (m, 1H), 4.48 (dd, J = 5.6, 9.2 Hz, 1H), 4.35 (q, J = 7.2 Hz, 3H), 4.15- 4.05 (m, 3H), 3.95-3.85 (m, 3H), 3.74 (s, 3H), 2.56 (s, 3H), 2.16-1.99 (m, 2H), 1.49 (t, J = 7.2 Hz, 3H), 1.41 (d, J = 7.2 Hz, 3H), 1.28 (s, 3H), 1.20-1.15 (m, 1H). |
| B | 115 | 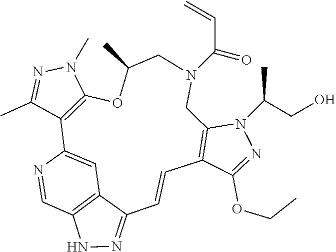 | 547.3 | ¹H NMR (400 MHz, DMSO-d6) δ ppm 8.98 (s, 1 H), 8.55 (br s, 1 H), 7.53 (br d, J = 16.47 Hz, 1 H), 7.23 (d, J = 16.47 Hz, 1 H), 7.06 (br d, J = 13.49 Hz, 1 H), 6.34 (dd, J = 16.35, 2.17 Hz, 1 H), 5.84 (br d, J = 11.66 Hz, 1 H), 5.51-5.65 (m, 1 H), 5.05 (br s, 1 H), 4.82 (br s, 1 H), 4.61 (br s, 1 H), 4.34-4.43 (m, 1 H), 4.24-4.35 (m, 1 H), 4.03 (br dd, J = 14.75, 5.83 Hz, 1 H), 3.63- 3.69 (m, 1 H), 3.61 (s, 3 H), 3.48-3.57 (m, 2 H), 2.55 (s, 3 H), 1.42 (t, J = 6.98 Hz, 3 H), 1.11 (br d, J = 6.40 Hz, 3 H), 1.02 (br d, J = 5.49 Hz, 3 H). |
| B | 116 | 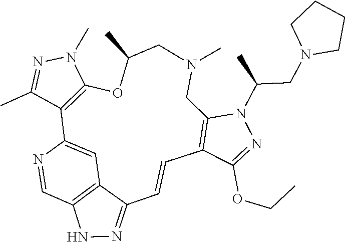 | 560.4 | ¹H NMR (499 MHz, DMSO-d6) δ ppm 9.59 (br s, 1 H) 9.01 (d, J = 1.10 Hz, 1 H) 8.40 (s, 1 H) 8.09 (br d, J = 4.38 Hz, 1 H) 7.20 (d, J = 17.25 Hz, 1 H) 4.84- 4.90 (m, 1 H) 4.79 (br s, 1 H) 4.29-4.41 (m, 4 H) 3.75-3.84 (m, 2 H) 3.64 (s, 3 H) 3.46-3.57 (m, 2 H) 3.26 (br d, J = 3.83 Hz, 1 H) 3.19 (ddd, J = 6.64, 4.18, 2.87 Hz, 1 H) 3.04 (br s, 1 H) 2.94 (br d, J = 6.57 Hz, 1 H) 2.47-2.49 (m, 3 H) 2.34- 2.47 (m, 3 H) 1.99 (br d, J = 3.01 Hz, 2 H) 1.82 (br d, J = 3.83 Hz, 2 H) 1.44 (t, J = 6.98 Hz, 3 H) 1.34 (br d, J = 6.57 Hz, 3 H) 1.07 (d, J = 6.30 Hz, 3 H); |
| B | 117 | 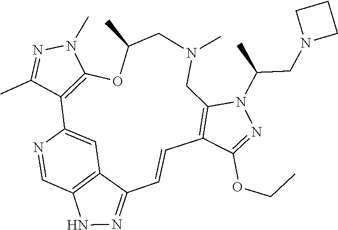 | 546.4 | ¹H NMR (400 MHz, DMSO-d6) δ ppm 10.30 (br s, 1 H) 8.99 (s, 1 H) 8.37 (s, 1 H) 7.91 (s, 1 H) 7.16 (d, J = 16.70 Hz, 1 H) 4.76-5.00 (m, 1 H) 4.71 (br s, 1 H) 4.27- 4.40 (m, 2 H) 3.96-4.18 (m, 3 H) 3.92 (s, 1 H) 3.65-3.83 (m, 3 H) 3.61 (s, 3 H) 3.48 (br d, J = 12.81 Hz, 1 H) 2.88- 3.30 (m, 3 H) 2.67-2.87 (m, 1 H) 2.45 (br s, 3 H) 2.22-2.36 (m, 2 H) 2.10- 2.22 (m, 1 H) 1.41 (t, J = 6.98 Hz, 3 H) 1.30 (br d, J = 5.72 Hz, 3 H) 1.03 (br d, J = 6.17 Hz, 3 H) |
| B | 118 | 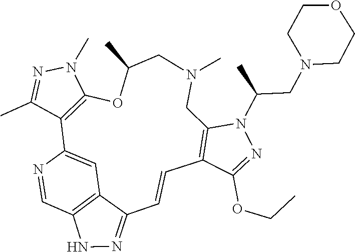 | 576.5 | ¹H NMR (400 MHz, DMSO-d₆) δ ppm (400 MHz, DMSO-d6) δ ppm 9.64 (br dd, J = 8.69, 4.35 Hz, 1 H) 8.95 (s, 1 H) 8.35 (s, 1 H) 8.14 (br d, J = 15.09 Hz, 1 H) 7.16 (d, J = 16.92 Hz, 1 H) 4.59-5.02 (m, 2 H) 4.22- 4.38 (m, 4 H) 3.91- 4.18 (m, 5 H) 3.78-3.91 (m, 4 H) 3.58-3.61 (m, 3 H) 3.44-3.54 (m, 1 H) 3.11 (br s, 2 H) 2.90- 3.02 (m, 3 H) 2.82 (br d, J = 4.35 Hz, 1 H) 2.20- 2.40 (m, 3 H) 1.40 (t, J = 6.98 Hz, 3 H) 1.30 (br d, J = 6.17 Hz, 3 H) 1.03 (d, J = 6.17 Hz, 3 H). |
| B | 119 | 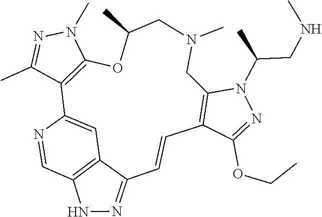 | 520.4 | ¹H NMR (400 MHz, DMSO-d6) δ ppm 8.97 (s, 1 H), 8.38 (s, 1 H), 8.16 (d, J = 17.15 Hz, 1 H), 7.17 (d, J = 16.92 Hz, 1 H), 4.70 (br dd, J = 13.72, 5.95 Hz, 2 H), 4.25-4.39 (m, 2 H), 3.89-4.00 (m, 1 H), 3.76-3.89 (m, 1 H), 3.62 (s, 3 H), 3.09 (br d, J = 9.61 Hz, 1 H), 2.93- 3.01 (m, 1 H), 2.83 (br dd, J = 14.29, 7.43 Hz, 1 H), 2.45-2.48 (m, 3 H), 2.34 (s, 3 H), 1.42 (t, J = 6.98 Hz, 3 H), 1.33 (d, J = 6.63 Hz, 3 H), 1.06 (br d, J = 6.17 Hz, 3 H) |
| C | 120 | 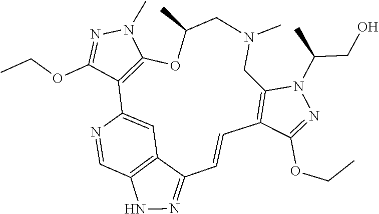 | 537.2 | ¹H NMR (400 MHz, MeOD-d₄) δ = 8.90 (s, 1H), 8.38 (s, 1H), 8.22 (d, J = 16.8 Hz, 1H), 7.24 (d, J = 16.8 Hz, 1H), 4.83-4.80 (m, 1H), 4.51-4.42 (m, 1H), 4.40- 4.32 (m, 2H), 4.30 (dd, J = 1.2, 7.2 Hz, 2H), 4.00-3.93 (m, 1H), 3.85 (s, 1H), 3.83-3.79 (m, 1H), 3.70 (d, J = 4.8 Hz, 1H), 3.63 (s, 3H), 2.92 (d, J = 1.6 Hz, 1H), 2.86 (d, J = 8.0 Hz, 1H), 2.42 (s, 3H), 1.48 (t, J = 7.2S Hz, 3H), 1.45-1.38 (m, 6H), 1.12 (d, J = 6.4 Hz, 3H). |
| B | 121 | 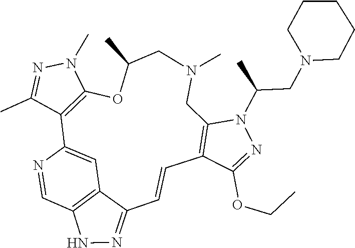 | 574.5 | ¹H NMR (499 MHz, DMSO-d6) δ ppm 13.13- 13.49 (m, 1 H) 9.17 (br d, J = 7.39 Hz, 1 H) 8.99 (d, J = 0.82 Hz, 1 H) 8.40 (s, 1 H) 8.20 (br d, J = 16.97 Hz, 1 H) 7.20 (d, J = 16.97 Hz, 1 H) 4.89-5.05 (m, 1 H) 4.71 (br s, 1 H) 4.27-4.40 (m, 2 H) 3.85-4.05 (m, 4 H) 3.74-3.82 (m, 2 H) 3.62-3.64 (m, 3 H) 3.45 (br d, J = 13.42 Hz, 2 H) 3.14 (br d, J = 11.77 Hz, 1 H) 2.83-3.04 (m, 4 H) 2.36 (br s, 3 H) 1.73- 1.87 (m, 2 H) 1.64 (br d, J = 7.94 Hz, 3 H) 1.44 (t, J = 6.98 Hz, 3 H) 1.38 (br d, J = 12.05 Hz, 1 H) 1.33 (d, J = 6.57 Hz, 3 H) 1.07 (d, J = 6.30 Hz, 3 H) |
| B | 122 | 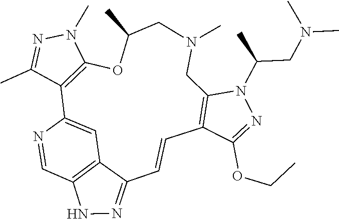 | 534.4 | ¹H NMR (499 MHz, METHANOL-d4) δ ppm 8.95 (s, 1 H), 8.44 (s, 1 H), 8.26 (d, J = 16.70 Hz, 1 H), 7.26 (d, J = 16.97 Hz, 1 H), 4.47-4.68 (m, 1 H), 4.33-4.40 (m, 2 H), 3.98 (d, J = 14.24 Hz, 1 H), 3.84 (d, J = 14.24 Hz, 1 H), 3.69 (s, 3 H), 3.02 (dd, J = 12.87, 9.03 Hz, 1 H), 2.93-2.98 (m, 1 H), 2.83-2.90 (m, 1 H), 2.49-2.55 (m, 4 H), 2.44 (s, 3 H), 2.21 (s, 6 H), 1.48 (t, J = 6.98 Hz, 3 H), 1.38 (d, J = 6.57 Hz, 3 H), 1.10 (d, J = 6.02 Hz, 3 H) |
| B | 123 | 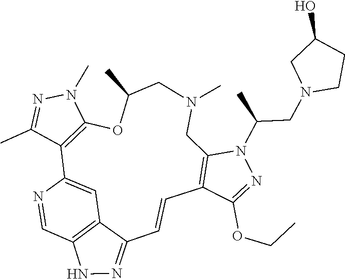 | 576.5 | ¹H NMR (400 MHz, DMSO-d6) δ ppm 9.86- 10.04 (m, 1 H) 9.34- 9.54 (m, 1 H) 8.89-9.04 (m, 1 H) 8.31-8.47 (m, 1 H) 7.95-8.24 (m, 1 H) 7.11-7.25 (m, 1 H) 4.82- 4.86 (m, 1 H) 4.66- 4.72 (m, 1 H) 4.27-4.41 (m, 4 H) 3.66-4.06 (m, 4 H) 3.59-3.64 (m, 3 H) 3.32-3.56 (m, 2 H) 3.13- 3.27 (m, 1 H) 2.75- 3.04 (m, 3 H) 2.45-2.45 (m, 3 H) 2.34 (br s, 3 H) 1.69-1.89 (m, 1 H) 1.37- 1.45 (m, 3 H) 1.24- 1.35 (m, 3 H) 1.00-1.08 (m, 3 H) |
| B | 124 | 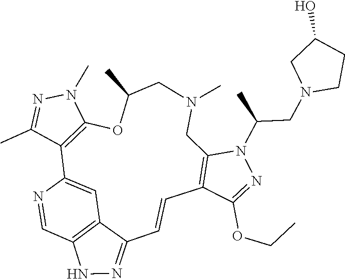 | 576.5 | ¹H NMR (400 MHz, DMSO-d6) δ ppm 9.93- 10.07 (m, 1 H) 9.82- 9.62 (m, 1 H) 9.01 (s, 1 H) 8.32-8.48 (m, 1 H) 7.98-8.27 (m, 1 H) 7.13- 7.27 (m, 1 H) 4.87- 4.91 (m, 1 H) 4.71-4.77 (m, 1 H) 4.29-4.39 (m, 4 H) 3.67-4.07 (m, 4 H) 3.62-3.67 (m, 3 H) 3.43- 3.68 (m, 2 H) 3.32- 3.46 (m, 2 H) 2.70-3.11 (m, 3 H) 2.49 (m, 3 H) 2.14-2.47 (m, 3 H) 1.83-1.96 (m, 1 H) 1.41- 1.46 (m, 3 H) 1.27- 1.36 (m, 3 H) 1.02-1.09 (m, 3 H). |
| B | 125 | 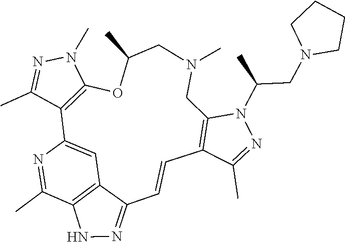 | 530.3 | ¹H NMR (400 MHz, DMSO-d₆) δ = 13.75- 13.06 (m, 1H), 8.96 (s, 1H), 7.29 (d, J = 17.2 Hz, 1H), 6.77 (d, J = 17.2 Hz, 1H), 4.27-4.06 (m, 2H), 4.00-3.73 (m, 2H), 3.42-3.41 (m, 2H), 3.23 (s, 3H), 2.94-2.70 (m, 3H), 2.62 (s, 3H), 2.46 (s, 4H), 2.38 (s, 3H), 2.17 (s, 3H), 2.12- 2.03 (m, 1H), 1.65 (s, 3H), 1.62 (s, 3H), 0.90 (d, J = 7.2 Hz, 3H). |
| B | 126 | 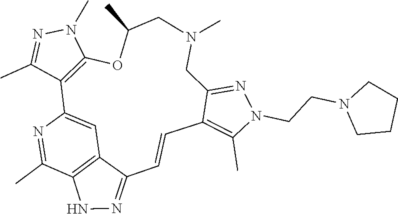 | 530.2 | ¹H NMR (400 MHz, DMSO-d₆) δ = 13.40 (s, 1H), 8.25-8.18 (m, 2H), 6.89 (d, J = 17.6 Hz, 1H), 4.72 (t, J = 6.4 Hz, 1H), 4.15 (dt, J = 1.6, 6.8 Hz, 2H), 3.87 (d, J = 13.2 Hz, 1H), 3.60 (s, 3H), 3.58-3.51 (m, 3H), 3.34-3.33 (m, 3H), 2.88 (d, J = 13.2 Hz, 1H), 2.81-2.76 (m, 2H), 2.74 (s, 3H), 2.71 (d, J = 7.6 Hz, 1H), 2.55 (s, 2H), 2.49-2.49 (m, 3H), 2.25 (s, 3H), 1.67 (t, J = 3.2 Hz, 4H), 0.99 (d, J = 6.4 Hz, 3H). |
| A | 127 | 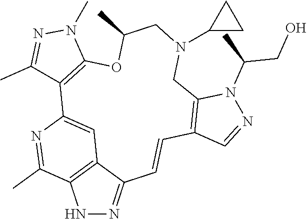 [SMILES: Cc1nn(C)c2c1-c1cc3c(n[nH]c3c(C)n1)/C=C/c1cnn([C@@H](C)CO)c1CN(C1CC1)C[C@H](C)O2] | 503.2 | ¹H NMR (400 MHz, MeOD-d₄) δ = 8.19 (s, 1H), 7.90 (s, 1H), 7.65 (d, J = 16.9 Hz, 1H), 7.05 (d, J = 16.8 Hz, 1H), 4.83-4.80 (m, 1H), 4.78-4.71 (m, 1H), 4.15- 3.99 (m, 2H), 3.80- 3.72 (m, 2H), 3.68 (s, 3H), 3.24 (dd, J = 7.2, 14.8 Hz, 1H), 3.05 (d, J = 14.4 Hz, 1H), 2.77 (s, 3H), 2.57 (s, 3H), 2.06- 1.97 (m, 1H), 1.51 (d, J = 6.4 Hz, 3H), 1.03 (d, J = 6.4 Hz, 3H), 0.58- 0.33 (m, 2H), 0.28-0.02 (m, 2H). |
| A | 128 | 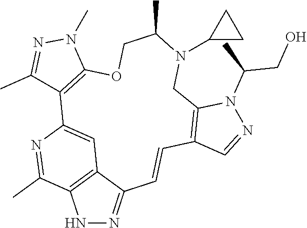 [SMILES: Cc1nn(C)c2c1-c1cc3c(n[nH]c3c(C)n1)/C=C/c1cnn([C@@H](C)CO)c1CN(C1CC1)[C@H](C)CO2] | 503.2 | ¹H NMR (400 MHz, MeOD-d₄) δ = 8.11 (s, 1H), 7.89 (s, 1H), 7.49- 7.42 (m, 1H), 7.05 (d, J = 16.8 Hz, 1H), 4.70- 4.64 (m, 1H), 4.48 (dd, J = 4.8, 8.8 Hz, 1H), 4.17- 4.09 (m, 2H), 3.91- 3.79 (m, 3H), 3.71 (s, 3H), 3.48 (d, J = 6.0 Hz, 1H), 2.76-2.73 (m, 3H), 2.56 (s, 3H), 2.11 (d, J = 3.2 Hz, 1H), 1.48 (d, J = 6.8 Hz, 3H), 1.41 (d, J = 6.8 Hz, 3H), 0.43-0.38 (m, 2H), 0.32-0.21 (m, 2H). |
| D | 129 | 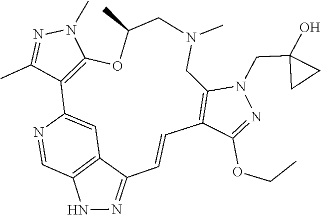 | 519.2 | ¹H NMR (400 MHz, MeOD-d₄) δ = 8.96 (s, 1H), 8.47 (s, 1H), 8.36 (d, J = 17.2 Hz, 1H), 7.29 (d, J = 17.2 Hz, 1H), 4.59 (br s, 3H), 4.39-4.32 (m, 2H), 4.28 (br d, J = 14.8 Hz, 1H), 3.99 (br d, J = 4.4 Hz, 2H), 3.70 (s, 3H), 3.00- 2.85 (m, 2H), 2.55 (s, 3H), 2.45 (s, 3H), 1.50 (t, J = 7.2 Hz, 3H), 1.37- 1.26 (m, 3H), 1.13 (d, J = 6.4 Hz, 3H). |
| C | 130 | 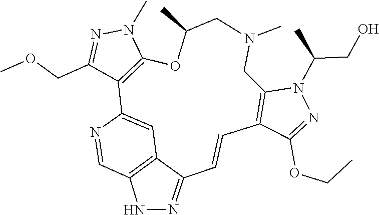 | 537.2 | ¹H NMR (400 MHz, MeOD-d₄) δ = 8.95 (s, 1H), 8.47 (s, 1H), 8.26 (d, J = 17.2 Hz, 1H), 7.26 (d, J = 16.8 Hz, 1H), 5.01 (d, J = 11.6 Hz, 1H), 4.80 (d, J = 11.6 Hz, 2H), 4.46 (d, J = 5.6 Hz, 1H), 4.36 (d, J = 5.6 Hz, 2H), 3.98 (d, J = 14.4 Hz, 1H), 3.87- 3.79 (m, 2H), 3.75 (s, 3H), 3.72-3.66 (m, 1H), 3.41 (s, 3H), 2.98-2.84 (m, 2H), 2.44 (s, 3H), 1.48 (t, J = 6.8 Hz, 3H), 1.40 (d, J = 6.4 Hz, 3H), 1.12 (d, J = 6.0 Hz, 3H). |
| B | 131 | 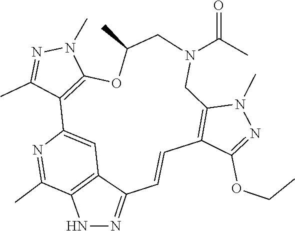 [SMILES: CCOc1nn(C)c2c1/C=C/c1n[nH]c3c(C)nc(cc13)-c1c(C)nn(C)c1O[C@@H](C)CN(C(C)=O)C2] | 505.2 | ¹H NMR (400 MHz, DMSO-d₆) δ = 13.51- 13.34 (m, 1H), 8.27 (br s, 1H), 7.42 (br d, J = 16.4 Hz, 1H), 7.28-7.17 (m, 1H), 5.29-5.04 (m, 2H), 4.37 (br d, J = 15.6 Hz, 1H), 4.28 (q, J = 6.8 Hz, 2H), 3.82-3.61 (m, 8H), 2.74 (s, 3H), 2.56 (s, 3H), 2.24 (br s, 3H), 1.42 (br t, J = 7.2 Hz, 3H), 1.12 (br s, 3H). |
| F | 132a | 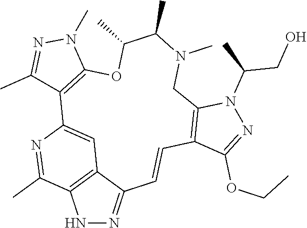 [SMILES: CCOc1nn([C@@H](C)CO)c2c1/C=C/c1n[nH]c3c(C)nc(cc13)-c1c(C)nn(C)c1O[C@H](C)[C@@H](C)N(C)C2] | 535.3 | ¹H NMR (400 MHz, DMSO-d6) δ = 13.27 (br s, 1H), 8.50 (br s, 1H), 8.30-8.16 (m, 1H), 7.29 (br d, J = 17.2 Hz, 1H), 4.80 (br d, J = 5.6 Hz, 1H), 4.38-4.25 (m, 4H), 4.08 (br d, J = 14.8 Hz, 1H), 3.95-3.86 (m, 1H), 3.66 (s, 3H), 3.57- 3.49 (m, 2H), 3.00-2.87 (m, 1H), 2.72 (s, 3H), 2.56 (s, 3H), 2.34 (br s, 3H), 1.45-1.39 (m, 6H), 1.31 (br d, J = 6.4 Hz, 3H), 1.21 (br d, J = 5.2 Hz, 3H |
| F | 132b | 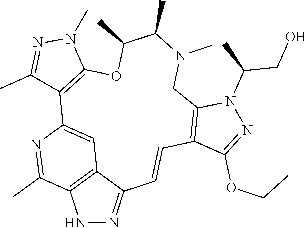 [SMILES: CCOc1nn([C@@H](C)CO)c2c1/C=C/c1n[nH]c3c(C)nc(cc13)-c1c(C)nn(C)c1O[C@@H](C)[C@@H](C)N(C)C2] | 535.2 | ¹H NMR (400 MHz, DMSO-d6) δ = 13.44- 13.19 (m, 1H), 8.34- 8.20 (m, 1H), 8.05-7.88 (m, 1H), 7.15 (br d, J = 17.2 Hz, 1H), 5.07-4.92 (m, 1H), 4.84-4.72 (m, 1H), 4.47-4.37 (m, 1H), 4.32-4.24 (m, 2H), 4.04- 3.84 (m, 2H), 3.60 (s, 3H), 3.52 (br s, 2H), 3.04 (br d, J = 6.4 Hz, 1H), 2.73 (s, 3H), 2.49- 2.48 (m, 3H), 2.32 (br s, 3H), 1.41 (br t, J = 6.8 Hz, 3H), 1.29 (br dd, J = 6.4, 15.6 Hz, 6H), 0.97 (br d, J = 6.0 Hz, 3H |
| A | 133 | 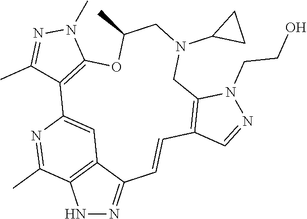 [SMILES: Cc1nn(C)c2c1-c1cc3c(n[nH]c3c(C)n1)/C=C/c1cnn(CCO)c1CN(C1CC1)C[C@H](C)O2] | 489.3 | ¹H NMR (400 MHz, MeOD-d₄) δ = 8.50 (br s, 2H), 8.24 (s, 1H), 7.87 (s, 1H), 7.67 (d, J = 17.2 Hz, 1H), 7.05 (d, J = 16.8 Hz, 1H), 4.45 (td, J = 6.4, 14.4 Hz, 1H), 4.26 (td, J = 4.8, 14.4 Hz, 1H), 4.13-4.00 (m, 2H), 3.92-3.84 (m, 2H), 3.66 (s, 3H), 3.46-3.38 (m, 2H), 3.23-3.04 (m, 2H), 2.77 (s, 3H), 2.55 (s, 3H), 2.19-2.12 (m, 1H), 1.03 (d, J = 6.4 Hz, 3H), 0.48-−0.08 (m, 4H |
| A | 134 | 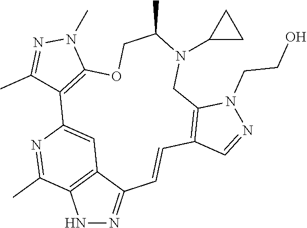 [SMILES: Cc1nn(C)c2c1-c1cc3c(n[nH]c3c(C)n1)/C=C/c1cnn(CCO)c1CN(C1CC1)[C@H](C)CO2] | 489.3 | ¹H NMR (400 MHz, MeOD-d₄) δ = 8.52 (br s, 6H), 8.31 (s, 1H), 7.92 (s, 1H), 7.61 (d, J = 16.8 Hz, 1H), 7.12 (d, J = 16.8 Hz, 1H), 4.46-4.39 (m, 2H), 4.28 (td, J = 5.2, 14.4 Hz, 2H), 4.16- 4.08 (m, 2H), 3.92 (br t, J = 5.2 Hz, 2H), 3.76 (s, 3H), 3.50-3.46 (m, 2H), 2.80 (s, 3H), 2.59 (s, 3H), 2.17-2.13 (m, 1H), 1.44 (d, J = 6.8 Hz, 3H), 0.51-0.11 (m, 4H) |
| G | 135 | 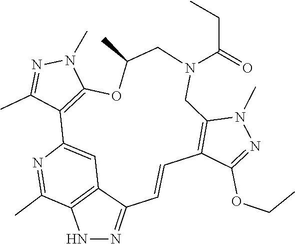 [SMILES: CCOc1nn(C)c2c1/C=C/c1n[nH]c3c(C)nc(cc13)-c1c(C)nn(C)c1O[C@@H](C)CN(C(=O)CC)C2] | 519.3 | ¹H NMR (400 MHz, MeOD-d₄) δ = 8.50 (br s, 2H), 8.24 (s, 1H), 7.87 (s, 1H), 7.67 (d, J = 17.2 Hz, 1H), 7.05 (d, J = 16.8 Hz, 1H), 4.45 (td, J = 6.4, 14.4 Hz, 1H), 4.26 (td, J = 4.8, 14.4 Hz, 1H), 4.13-4.00 (m, 2H), 3.92-3.84 (m, 2H), 3.66 (s, 3H), 3.46-3.38 (m, 2H), 3.23-3.04 (m, 2H), 2.77 (s, 3H), 2.55 (s, 3H), 2.19-2.12 (m, 1H), 1.03 (d, J = 6.4 Hz, 3H), 0.48-−0.08 (m, 4H). |
| G | 136 | 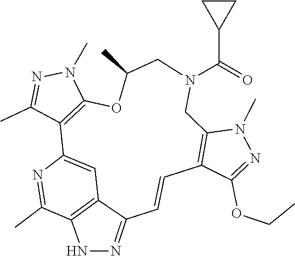 [SMILES: CCOc1nn(C)c2c1/C=C/c1n[nH]c3c(C)nc(cc13)-c1c(C)nn(C)c1O[C@@H](C)CN(C(=O)C1CC1)C2] | 531.2 | ¹H NMR (400 MHz, MeOD-d₄) δ = 8.50 (br s, 2H), 8.24 (s, 1H), 7.87 (s, 1H), 7.67 (d, J = 17.2 Hz, 1H), 7.05 (d, J = 16.8 Hz, 1H), 4.45 (td, J = 6.4, 14.4 Hz, 1H), 4.26 (td, J = 4.8, 14.4 Hz, 1H), 4.13-4.00 (m, 2H), 3.92-3.84 (m, 2H), 3.66 (s, 3H), 3.46-3.38 (m, 2H), 3.23-3.04 (m, 2H), 2.77 (s, 3H), 2.55 (s, 3H), 2.19-2.12 (m, 1H), 1.03 (d, J = 6.4 Hz, 3H), 0.48-−0.08(m, 4H). |
| G | 137 | 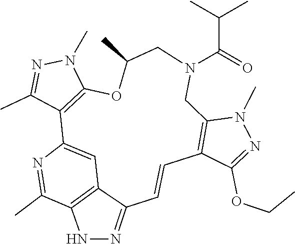 [SMILES: CCOc1nn(C)c2c1/C=C/c1n[nH]c3c(C)nc(cc13)-c1c(C)nn(C)c1O[C@@H](C)CN(C(=O)C(C)C)C2] | 533.2 | ¹H NMR (400 MHz, DMSO-d₆) δ = 13.53- 13.30 (m, 1H), 8.52- 8.23 (m, 1H), 7.51-7.36 (m, 1H), 7.21 (d, J = 16.0 Hz, 1H), 5.53-5.24 (m, 1H), 5.14-4.98 (m, 1H), 4.45-4.32 (m, 1H), 4.29 (q, J = 6.4 Hz, 2H), 3.88-3.76 (m, 1H), 3.63 (s, 8H), 2.75 (s, 3H), 2.57 (s, 6H), 1.42 (t, J = 7.2 Hz, 3H), 1.27-1.08 (m, 6H). |
| G | 138 | 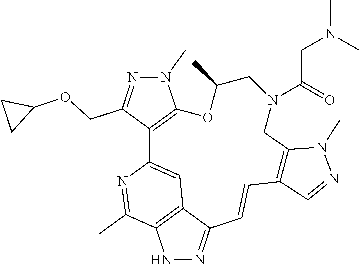 [SMILES: Cc1nc2cc3c(n[nH]c13)/C=C/c1cnn(C)c1CN(C(=O)CN(C)C)C[C@H](C)Oc1c-2c(COC2CC2)nn1C] | 560.4 | ¹H NMR (400 MHz, DMSO-d₆) δ = 13.72- 13.50 (m, 1H), 7.31 (s, 1H), 6.95-6.88 (m, 2H), 6.50 (s, 1H), 5.75-4.54 (m, 2H), 4.21 (d, J = 12.8 Hz, 1H), 4.09-3.91 (m, 1H), 3.79-3.59 (m, 1H), 3.18 (s, 3H), 3.06 (d, J = 14.4 Hz, 1H), 2.75-2.68 (m, 1H), 2.56 (s, 3H), 2.35-2.29 (m, 1H), 2.25 (s, 9H), 0.96- 0.82 (m, 2H), 0.81 (s, 2H), 0.65-0.44 (m, 3H), 0.41-0.29 (m, 2H). |
| A | 139 | 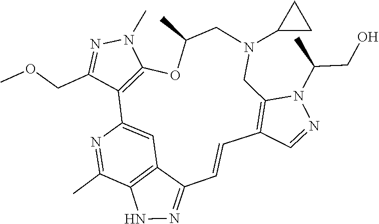 | 533.2 | ¹H NMR (400 MHz, MeOD-d₄) δ = 8.32- 8.20 (m, 1H), 7.89 (s, 1H), 7.69 (s, 1H), 7.04 (d, J = 16.8 Hz, 1H), 5.01-4.93 (m, 2H), 4.58 (s, 2H), 4.13-4.00 (m, 2H), 3.97-3.84 (m, 1H), 3.78-3.72 (m, 4H), 3.48- 3.42 (m, 3H), 3.30- 3.15 (m, 1H), 3.04 (d, J = 14.4 Hz, 1H), 2.78 (s, 3H), 2.09-1.95 (m, 1H), 1.55-1.42 (m, 3H), 1.13- 1.03 (m, 3H), 0.61- 0.35 (m, 2H), 0.34-0.04 (m, 2H). |
| A | 140 | 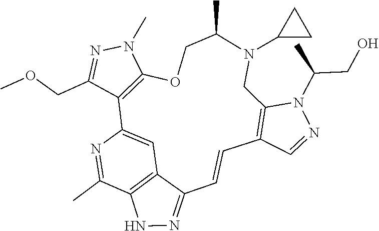 | 533.2 | ¹H NMR (400 MHz, MeOD-d₄) δ = 8.20 (s, 1H), 7.90 (s, 1H), 7.52 (d, J = 16.8 Hz, 1H), 7.08 (d, J = 16.8 Hz, 1H), 5.07 (d, J = 11.2 Hz, 1H), 4.71-4.65 (m, 1H), 4.60-4.54 (m, 2H), 4.21-4.14 (m, 2H), 3.91 (d, J = 14.4 Hz, 1H), 3.83 (t, J = 6.8 Hz, 2H), 3.79 (s, 3H), 3.56-3.49 (m, 1H), 3.44 (s, 3H), 2.78 (s, 3H), 2.17-2.08 (m, 1H), 1.48 (d, J = 6.8 Hz, 3H), 1.43 (d, J = 6.8 Hz, 3H), 0.42 (d, J = 5.6 Hz, 2H), 0.34-0.25 (m, 2H). |
| G | 141 | 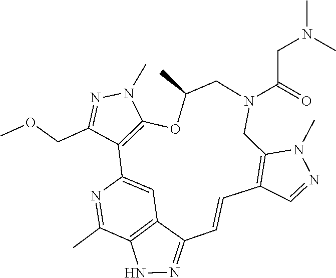 | 548.2 | ¹H NMR (400 MHz, MeOD-d₄) δ = 8.34- 8.19 (m, 1H), 7.87 (s, 1H), 7.38 (d, J = 16.4 Hz, 1H), 7.10 (d, J = 16.4 Hz, 1H), 5.42-5.22 (m, 1H), 5.05 (d, J = 3.2 Hz, 1H), 4.99-4.90 (m, 3H), 4.69-4.51 (m, 1H), 4.43-4.29 (m, 1H), 3.88 (s, 3H), 3.73 (s, 4H), 3.49 (s, 3H), 2.84 (s, 3H), 2.73 (s, 3H), 2.39 (s, 6H), 1.13 (s, 3H). |
| B | 142 | 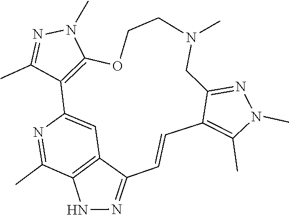 | 433.1 | ¹H NMR (400 MHz, MeOD-d₄) δ = 8.38 (s, 1H), 8.10 (d, J = 17.6 Hz, 1H), 6.96 (d, J = 17.2 Hz, 1H), 4.29-4.20 (m, 2H), 3.80 (s, 3H), 3.72 (s, 5H), 3.01-2.95 (m, 2H), 2.77 (s, 3H), 2.60 (s, 3H), 2.54 (s, 3H), 2.23 (s, 3H). |
| G | 143 | 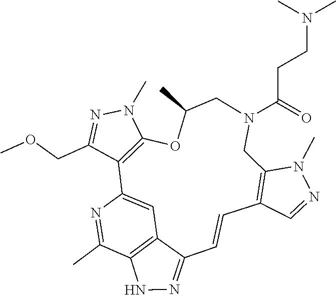 | 548.2 | ¹H NMR (400 MHz, MeOD-d₄) δ = 8.34- 8.19 (m, 1H), 7.87 (s, 1H), 7.38 (d, J = 16.4 Hz, 1H), 7.10 (d, J = 16.4 Hz, 1H), 5.42-5.22 (m, 1H), 5.05 (d, J = 3.2 Hz, 1H), 4.99-4.90 (m, 3H), 4.69-4.51 (m, 1H), 4.43-4.29 (m, 1H), 3.88 (s, 3H), 3.73 (s, 4H), 3.49 (s, 3H), 2.84 (s, 3H), 2.73 (s, 3H), 2.39 (s, 6H), 1.13 (s, 3H). |
| B | 144 | 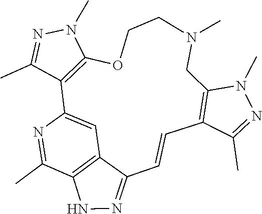 | 433.3 | ¹H NMR (400 MHz, MeOH-d₄) δ = 8.32 (br s, 1H), 8.15-8.09 (m, 1H), 6.98 (br d, J = 17.2 Hz, 1H), 4.22-4.14 (m, 2H), 3.79 (br s, 2H), 3.77 (s, 3H), 3.69 (s, 3H), 3.02-2.96 (m, 2H), 2.75 (s, 3H), 2.59 (s, 3H), 2.46 (s, 3H), 2.22 (s, 3H). |
| C | 145 | 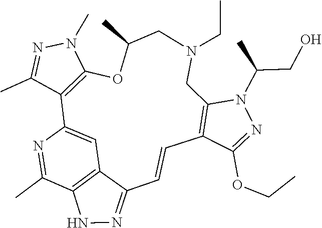 | 535.3 | ¹H NMR (400 MHz, MeOD-d₄) δ = 8.32 (s, 1H), 8.10 (d, J = 16.8 Hz, 1H), 7.25 (d, J = 17.2 Hz, 1H), 4.80 (br s, 1H), 4.54-4.43 (m, 1H), 4.42-4.31 (m, 2H), 4.05- 3.91 (m, 2H), 3.85- 3.77 (m, 1H), 3.74-3.62 (m, 4H), 3.11-3.00 (m, 1H), 2.89-2.81 (m, 1H), 2.79 (s, 3H), 2.77-2.71 (m, 1H), 2.71-2.62 (m, 1H), 2.56 (s, 3H), 1.48 (t, J = 7.2 Hz, 3H), 1.41 (d, J = 6.4 Hz, 3H), 1.09 (d, J = 6.4 Hz, 3H), 1.00 (t, J = 7.2 Hz, 3H). |
| C | 146 | 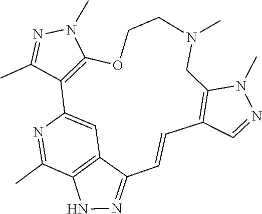 | 419.3 | ¹H NMR (400 MHz, MeOD-d₄) δ = 8.37 (s, 1H), 8.20 (d, J = 17.2 Hz, 1H), 7.84 (s, 1H), 7.06 (d, J = 17.2 Hz, 1H), 4.23 (s, 2H), 3.93- 3.88 (m, 2H), 3.84 (s, 3H), 3.71 (s, 3H), 3.06 (s, 2H), 2.76 (s, 3H), 2.61 (s, 3H), 2.27 (s, 3H). |
| C | 147 | 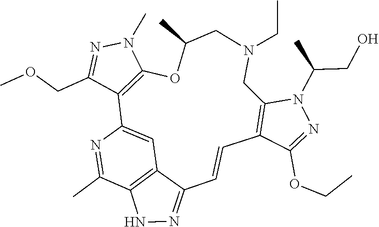 | 565.3 | ¹H NMR (400 MHz, MeOD-d₄) δ = 8.38 (s, 1H), 8.11 (d, J = 16.8 Hz, 1H), 7.26 (d, J = 16.8 Hz, 1H), 5.03 (d, J = 11.2 Hz, 1H), 4.82 (s, 2H), 4.53-4.44 (m, 1H), 4.40-4.32 (m, 2H), 4.03- 3.92 (m, 2H), 3.84- 3.77 (m, 1H), 3.75-3.75 (m, 1H), 3.74 (s, 2H), 3.71-3.66 (m, 1H), 3.43 (s, 3H), 3.12-3.04 (m, 1H), 2.86 (d, J = 15.2 Hz, 1H), 2.80 (s, 3H), 2.79-2.73 (m, 1H), 2.71- 2.63 (m, 1H), 1.48 (t, J = 7.2 Hz, 3H), 1.41 (d, J = 6.8 Hz, 3H), 1.11 (d, J = 6.4 Hz, 3H), 1.01 (t, J = 7.2 Hz, 3H). |
| C | 148 | 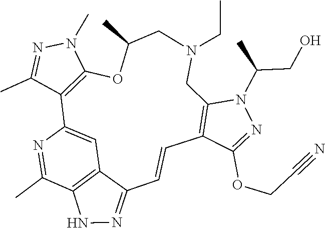 | 546.2 | ¹H NMR (400 MHz, MeOD-d₄) δ = 8.32 (s, 1H), 8.12 (d, J = 17.2 Hz, 1H), 7.17 (d, J = 16.8 Hz, 1H), 5.20-5.14 (m, 2H), 4.59 (s, 1H), 4.54 (dd, J = 2.8, 4.4 Hz, 1H), 4.02 (s, 2H), 3.87- 3.77 (m, 1H), 3.74-3.65 (m, 4H), 3.12-3.02 (m, 1H), 2.85 (d, J = 15.2 Hz, 1H), 2.80 (s, 3H), 2.78-2.73 (m, 1H), 2.66 (s, 1H), 2.57 (s, 3H), 1.44 (d, J = 6.8 Hz, 3H), 1.09 (d, J = 6.4 Hz, 3H), 1.01 (t, J = 7.2 Hz, 3H). |
| A | 149 | 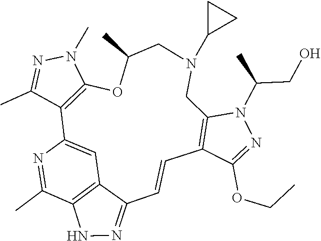 | 547.3 | ¹H NMR (400 MHz, MeOD) δ = 8.25 (s, 1H), 7.64-7.56 (m, 1H), 7.43- 7.39 (m, 1H), 7.29 (d, J = 7.6 Hz, 1H), 4.68 (d, J = 7.6 Hz, 1H), 4.54 (d, J = 5.6 Hz, 1H), 4.42- 4.35 (m, 2H), 4.06-3.99 (m, 2H), 3.78-3.74 (m, 1H), 3.70 (s, 3H), 3.27- 3.21 (m, 1H), 3.10-3.02 (m, 1H), 2.79 (s, 3H), 2.58 (s, 3H), 2.03-2.01 (m, 1H), 1.50-1.47 (m, 3H), 1.47-1.44 (m, 3H), 1.06 (s, 3H), 0.59-0.51 (m, 1H), 0.44-0.36 (m, 1H), 0.34-0.21 (m, 2H). |
| B | 150 | 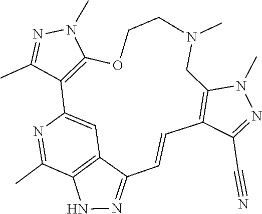 | 444.2 | ¹H NMR (400 MHz, DMSO-d₆) δ = 8.34 (s, 1H), 8.16 (d, J = 17.6 Hz, 1H), 7.49 (d, J = 17.6 Hz, 1H), 4.24 (br s, 2H), 3.99-3.96 (m, 2H), 3.92 (s, 3H), 3.68 (s, 3H), 3.02 (br s, 2H), 2.76 (s, 3H), 2.55 (s, 3H), 2.18 (s, 3H). |
| A | 151 | 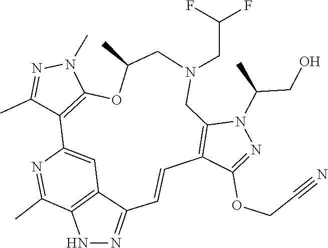 [SMILES: Cc1nn(C)c2c1-c1cc3c(n[nH]c3c(C)n1)/C=C/c1c(OCC#N)nn([C@@H](C)CO)c1CN(CC(F)F)C[C@H](C)O2] | 582.3 | ¹H NMR (400 MHz, MeOH-d₄) δ = 8.26 (s, 1H), 7.94 (d, J = 17.2 Hz, 1H), 7.19 (d, J = 17.2 Hz, 1H), 5.96 (tt, J = 4.0, 55.6 Hz, 1H), 5.18 (s, 2H), 4.92-4.88 (m, 1H), 4.64-4.45 (m, 1H), 4.26-4.19 (m, 1H), 4.16- 4.01 (m, 1H), 3.85- 3.76 (m, 1H), 3.72-3.70 (m, 1H), 3.68 (s, 3H), 3.29-3.21 (m, 1H), 3.12- 2.98 (m, 3H), 2.80 (s, 3H), 2.56 (s, 3H), 1.44 (d, J = 6.4 Hz, 3H), 1.08 (d, J = 6.4 Hz, 3H). |
| A | 152 | 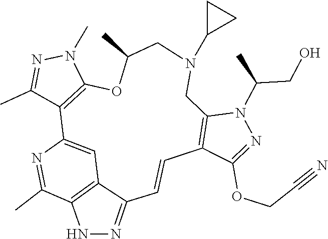 [SMILES: Cc1nn(C)c2c1-c1cc3c(n[nH]c3c(C)n1)/C=C/c1c(OCC#N)nn([C@@H](C)CO)c1CN(C1CC1)C[C@H](C)O2] | 558.3 | ¹H NMR (400 MHz, MeOD-d₄) δ = 8.28- 8.21 (m, 1H), 7.66-7.54 (m, 1H), 7.16 (d, J = 16.8 Hz, 1H), 5.18 (s, 2H), 4.79-4.69 (m, 2H), 4.10-4.00 (m, 2H), 3.83- 3.72 (m, 2H), 3.70 (s, 3H), 3.20 (s, 1H), 3.11- 3.01 (m, 1H), 2.79 (s, 3H), 2.61-2.55 (m, 3H), 2.08-1.99 (m, 1H), 1.51- 1.45 (m, 3H), 1.05 (d, J = 6.4 Hz, 3H), 0.60- 0.36 (m, 2H), 0.35-0.15 (m, 2H). |
| A | 153 | 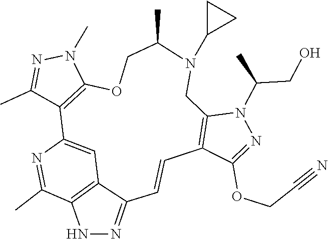 [SMILES: Cc1nn(C)c2c1-c1cc3c(n[nH]c3c(C)n1)/C=C/c1c(OCC#N)nn([C@@H](C)CO)c1CN(C1CC1)[C@H](C)CO2] | 558.4 | ¹H NMR (400 MHz, MeOD-d₄) δ = 8.14 (s, 1H), 7.50-7.41 (m, 1H), 7.20 (d, J = 16.8 Hz, 1H), 5.17 (d, J = 0.8 Hz, 2H), 4.65-4.47 (m, 3H), 4.21- 4.11 (m, 2H), 3.93- 3.83 (m, 2H), 3.75 (s, 3H), 2.78 (s, 3H), 2.57 (s, 3H), 2.17-2.10 (m, 1H), 1.48-1.43 (m, 6H), 1.13-1.06 (m, 1H), 0.48- 0.39 (m, 2H), 0.36- 0.27 (m, 2H). |
| A | 154 | 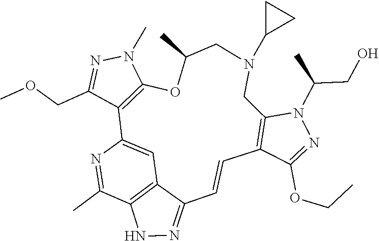 | 577.3 | ¹H NMR (400 MHz, MeOD-d₄) δ = 8.27 (s, 1H), 7.58 (d, J = 16.4 Hz, 1H), 7.23 (d, J = 16.4 Hz, 1H), 4.74-4.64 (m, 1H), 4.42-4.33 (m, 2H), 4.00 (br d, J = 9.6 Hz, 2H), 3.81-3.77 (m, 1H), 3.74 (s, 3H), 3.70 (br d, J = 4.8 Hz, 1H), 3.44 (s, 3H), 3.28-3.21 (m, 1H), 2.79 (s, 3H), 2.05-1.99 (m, 1H), 1.52- 1.43 (m, 6H), 1.36- 1.26 (m, 4H), 1.07 (d, J = 6.4 Hz, 3H), 0.60- 0.47 (m, 1H), 0.44-0.21 (m, 3H). |
| A | 155 | 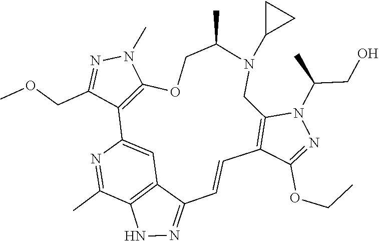 | 577.3 | ¹H NMR (400 MHz, MeOD-d₄) δ = 8.17 (s, 1H), 7.41-7.34 (m, 1H), 7.29-7.23 (m, 1H), 4.57 (br d, J = 6.4 Hz, 1H), 4.54-4.49 (m, 1H), 4.40- 4.31 (m, 2H), 4.14- 4.02 (m, 2H), 3.87-3.80 (m, 2H), 3.78 (s, 3H), 3.45 (s, 3H), 2.78-2.72 (m, 3H), 2.16-2.06 (m, 1H), 1.48 (t, J = 7.2 Hz, 3H), 1.43-1.38 (m, 6H), 1.33-1.28 (m, 4H), 0.51- 0.33 (m, 4H). |
| A | 156 | 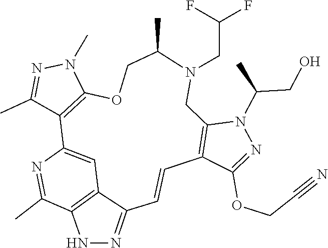 [SMILES: Cc1nn(C)c2c1-c1cc3c(n[nH]c3c(C)n1)/C=C/c1c(OCC#N)nn([C@@H](C)CO)c1CN(CC(F)F)[C@H](C)CO2] | 582.3 | ¹H NMR (400 MHz, MeOH-d₄) δ = 8.22 (s, 1H), 7.59 (d, J = 16.8 Hz, 1H), 7.24 (d, J = 16.8 Hz, 1H), 5.92-5.54 (m, 1H), 5.18 (d, J = 2.0 Hz, 2H), 4.66-4.59 (m, 1H), 4.42 (dd, J = 4.4, 9.2 Hz, 1H), 4.21-4.09 (m, 2H), 3.99-3.90 (m, 2H), 3.77 (dd, J = 4.8, 11.2 Hz, 1H), 3.73 (s, 3H), 3.42 (dd, J = 5.6, 11.2 Hz, 1H), 3.09-2.97 (m, 2H), 2.78 (s, 3H), 2.58 (s, 3H), 1.41 (t, J = 7.2 Hz, 6H). |
| A | 157 | 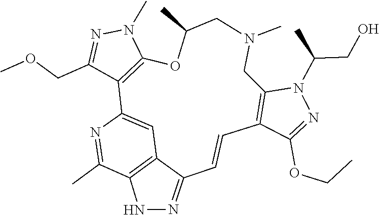 | 551.3 | ¹H NMR (400 MHz, MeOD-d₄) δ = 8.31 (s, 1H), 8.26 (d, J = 17.2 Hz, 1H), 7.25 (d, J = 16.8 Hz, 1H), 5.06 (d, J = 11.2 Hz, 1H), 4.95- 4.91 (m, 1H), 4.81 (d, J = 11.2 Hz, 1H), 4.49- 4.42 (m, 1H), 4.39-4.31 (m, 2H), 3.98 (d, J = 14.2 Hz, 1H), 3.87-3.80 (m, 2H), 3.74 (s, 3H), 3.71-3.65 (m, 1H), 3.42 (s, 3H), 2.98-2.85 (m, 2H), 2.83-2.79 (m, 3H), 2.44 (s, 3H), 1.48 (t, J = 7.2 Hz, 3H), 1.40 (d, J = 6.4 Hz, 3H), 1.12 (d, J = 6.4 Hz, 3H). |
| A | 158 | 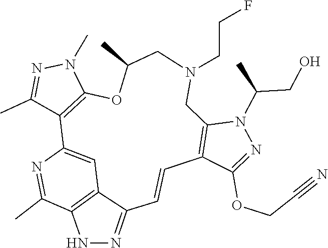 [SMILES: Cc1nn(C)c2c1-c1cc3c(n[nH]c3c(C)n1)/C=C/c1c(OCC#N)nn([C@@H](C)CO)c1CN(CCF)C[C@H](C)O2] | 564.4 | ¹H NMR (400 MHz, DMSO-d₆) δ 13.39 (s, 1H), 8.25 (s, 1H), 7.93 (d, J = 17.2 Hz, 1H), 7.09 (d, J = 17.2 Hz, 1H), 5.24 (s, 2H), 4.85 (t, J = 5.2 Hz, 1H), 4.79- 4.69 (m, 1H), 4.61- 4.39 (m, 3H), 4.11-4.04 (m, 2H), 3.62 (s, 3H), 3.56 (t, J = 6.0 Hz, 2H), 3.14-2.99 (m, 2H), 2.96- 2.83 (m, 2H), 2.74 (s, 3H), 2.52 (d, J = 1.2 Hz, 3H), 1.35 (d, J = 6.4 Hz, 3H), 1.03 (d, J = 6.4 Hz, 3H). |
| A | 159 | 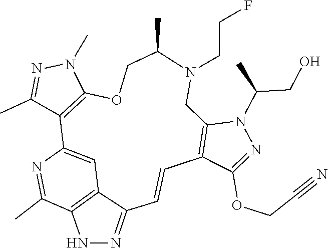 [SMILES: Cc1nn(C)c2c1-c1cc3c(n[nH]c3c(C)n1)/C=C/c1c(OCC#N)nn([C@@H](C)CO)c1CN(CCF)[C@H](C)CO2] | 564.4 | ¹H NMR (400 MHz, DMSO-d₆) δ 13.54- 13.36 (m, 1H), 8.18 (s, 1H), 7.56 (d, J = 16.8 Hz, 1H), 7.14 (d, J = 16.8 Hz, 1H), 5.25 (s, 2H), 4.81 (t, J = 5.6 Hz, 1H), 4.54 (d, J = 6.4 Hz, 1H), 4.52-4.45 (m, 2H), 4.36 (d, J = 1.2 Hz, 1H), 4.23 (t, J = 6.4 Hz, 1H), 4.14 (d, J = 14.4 Hz, 1H), 4.05 (dd, J = 7.2, 8.4 Hz, 1H), 3.90-3.83 (m, 1H), 3.73 (s, 1H), 3.68 (s, 3H), 3.64- 3.59 (m, 1H), 3.04-2.82 (m, 2H), 2.75 (s, 3H), 2.54 (s, 3H), 1.32 (dd, J = 6.8, 10.4 Hz, 6H). |
| C | 160 | 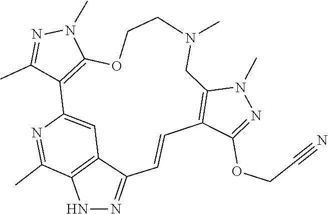 [SMILES: Cc1nn(C)c2c1-c1cc3c(n[nH]c3c(C)n1)/C=C/c1c(OCC#N)nn(C)c1CN(C)CCO2] | 474.3 | ¹H NMR (400 MHz, DMSO-d₆) δ = 13.40 (s, 1H), 8.34 (s, 1H), 8.05 (d, J = 17.2 Hz, 1H), 7.12 (d, J = 17.2 Hz, 1H), 5.23 (s, 2H), 4.24 (s, 2H), 3.86 (s, 2H), 3.72 (s, 3H), 3.68 (s, 3H), 3.00-2.98 (m, 2H), 2.73 (s, 3H), 2.54 (s, 3H), 2.20 (s, 3H). |
| C | 161 | 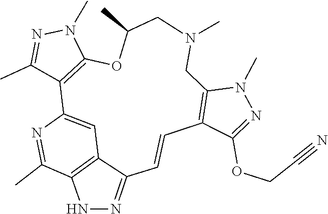 [SMILES: Cc1nn(C)c2c1-c1cc3c(n[nH]c3c(C)n1)/C=C/c1c(OCC#N)nn(C)c1CN(C)C[C@H](C)O2] | 488.4 | ¹H NMR (400 MHz, MeOD-d₄) δ = 8.31- 8.14 (m, 2H), 7.24-7.07 (m, 1H), 5.14 (s, 2H), 4.93 (s, 1H), 3.99-3.94 (m, 1H), 3.88-3.82 (m, 1H), 3.76 (s, 3H), 3.68 (s, 3H), 2.97-2.87 (m, 2H), 2.80 (s, 3H), 2.58- 2.53 (m, 3H), 2.43 (s, 3H), 1.16-1.07 (m, 3H). |
| A | 162 | 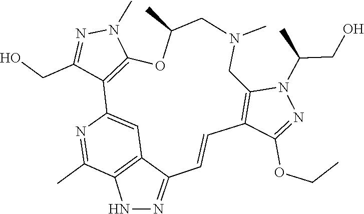 | 537.2 | ¹H NMR (400 MHz, MeOD-d₄) δ = 8.41 (s, 1H), 8.33-8.20 (m, 1H), 7.36-7.19 (m, 1H), 4.72- 4.65 (m, 2H), 4.61 (s, 1H), 4.53 (d, J = 13.2 Hz, 1H), 4.46 (dd, J = 7.2, 11.6 Hz, 1H), 4.36 (dt, J = 4.0, 6.8 Hz, 2H), 4.01-3.94 (m, 1H), 3.86- 3.79 (m, 2H), 3.71 (s, 3H), 3.00-2.93 (m, 1H), 2.90 (d, J = 8.0 Hz, 1H), 2.82 (s, 3H), 2.43 (s, 3H), 1.49 (t, J = 6.8 Hz, 3H), 1.40 (d, J = 6.8 Hz, 3H), 1.13 (d, J = 6.4 Hz, 3H). |
| C | 163 | 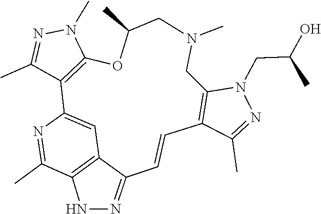 | 491.2 | ¹H NMR (400 MHz, MeOD-d₄) δ = 8.45 (d, J = 17.6 Hz, 1H), 8.24 (s, 1H), 6.99 (d, J = 17.6 Hz, 1H), 4.92-4.88 (m, 1H), 4.13-4.02 (m, 3H), 4.01- 3.95 (m, 1H), 3.93- 3.86 (m, 1H), 3.68 (s, 3H), 2.97-2.84 (m, 2H), 2.80 (s, 3H), 2.55 (s, 3H), 2.49 (s, 3H), 2.38 (s, 3H), 1.25-1.18 (m, 3H), 1.08 (d, J = 6.4 Hz, 3H) |
| G | 164 | 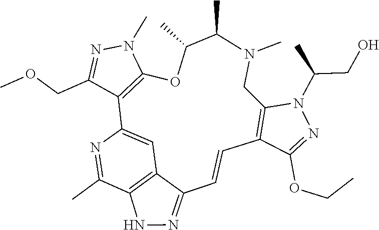 | 565.2 | ¹H NMR (400 MHz, DMSO-d₆) δ = 13.31 (s, 1H), 8.51 (s, 1H), 8.23 (d, J = 16.0 Hz, 1H), 7.29 (d, J = 17.2 Hz, 1H), 4.97 (d, J = 10.8 Hz, 1H), 4.84-4.73 (m, 2H), 4.43-4.34 (m, 2H), 4.29 (ttd, J = 3.2, 6.8, 10.4 Hz, 2H), 4.08 (d, J = 15.2 Hz, 1H), 3.91 (d, J = 15.2 Hz, 1H), 3.72 (s, 3H), 3.53 (s, 2H), 3.31 (s, 3H), 2.93 (d, J = 6.4 Hz, 1H), 2.74 (s, 3H), 2.34 (s, 3H), 1.46- 1.40 (m, 6H), 1.32 (d, J = 6.4 Hz, 3H), 1.22 (d, J = 6.0 Hz, 3H). |
| G | 165 | 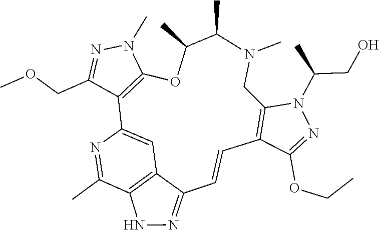 | 565.2 | ¹H NMR (400 MHz, DMSO-d₆) δ = 13.34 (s, 1H), 8.27 (d, J = 16.4 Hz, 1H), 7.99 (s, 1H), 7.15 (d, J = 17.2 Hz, 1H), 5.13-4.96 (m, 2H), 4.78 (t, J = 5.2 Hz, 1H), 4.57 (d, J = 10.8 Hz, 1H), 4.47-4.38 (m, 1H), 4.36-4.20 (m, 2H), 4.06- 3.95 (m, 1H), 3.94- 3.86 (m, 1H), 3.66 (s, 3H), 3.59-3.58 (m, 1H), 3.25 (s, 3H), 3.10-3.01 (m, 1H), 2.75 (s, 3H), 2.33 (s, 3H), 1.41 (t, J = 7.2 Hz, 3H), 1.29 (dd, J = 6.8, 13.2 Hz, 6H), 0.97 (d, J = 6.4 Hz, 3H). |
| — | 166 | 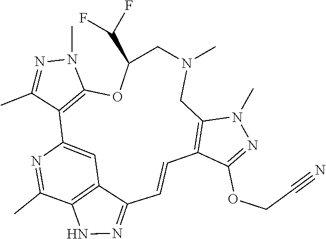 [SMILES: Cc1nn(C)c2c1-c1cc3c(n[nH]c3c(C)n1)/C=C/c1c(OCC#N)nn(C)c1CN(C)C[C@H](C(F)F)O2] | 524.2 | ¹H NMR (400 MHz, MeOD-d₄) δ = 8.15 (d, J = 17.2 Hz, 1H), 8.01 (s, 1H), 7.13 (d, J = 17.2 Hz, 1H), 6.05-5.69 (m, 1H), 5.14 (s, 2H), 4.83- 4.74 (m, 1H), 3.98-3.91 (m, 1H), 3.90-3.83 (m, 1H), 3.74 (s, 3H), 3.70 (s, 3H), 3.14-3.02 (m, 2H), 2.79 (s, 3H), 2.47 (s, 3H), 2.36 (s, 3H). |
| — | 167 | 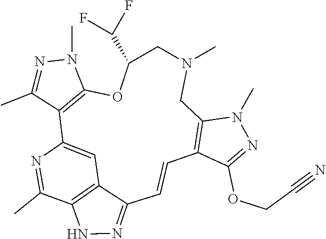 [SMILES: Cc1nn(C)c2c1-c1cc3c(n[nH]c3c(C)n1)/C=C/c1c(OCC#N)nn(C)c1CN(C)C[C@@H](C(F)F)O2] | 524.2 | ¹H NMR (400 MHz, MeOD-d₄) δ = 8.15 (d, J = 17.2 Hz, 1H), 8.00 (s, 1H), 7.14 (d, J = 17.2 Hz, 1H), 6.05-5.64 (m, 1H), 5.15 (s, 2H), 4.83- 4.72 (m, 1H), 3.98-3.90 (m, 1H), 3.90-3.82 (m, 1H), 3.75 (s, 3H), 3.70 (s, 3H), 3.14-3.01 (m, 2H), 2.79 (s, 3H), 2.47 (s, 3H), 2.35 (s, 3H). |
| C | 168 | 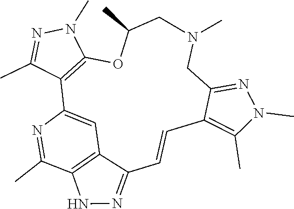 | 447.1 | ¹H NMR (400 MHz, CDCl₃) δ = 8.42 (br d, J = 17.6 Hz, 1H), 8.30 (s, 1H), 6.97 (d, J = 17.6 Hz, 1H), 4.88 (br d, J = 4.0 Hz, 1H), 3.94-3.86 (m, 1H), 3.81 (s, 3H), 3.79-3.73 (m, 1H), 3.71 (s, 3H), 2.83 (s, 3H), 2.80 (br s, 2H), 2.63 (s, 3H), 2.53 (s, 3H), 2.42 (s, 3H), 1.07 (d, J = 6.4 Hz, 3H). |
| C | 169 | 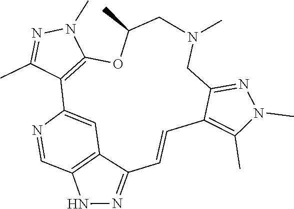 | 433.3 | ¹H NMR (400 MHz, DMSO-d₆) δ = 8.99 (s, 1H), 8.38 (s, 1H), 8.25 (d, J = 17.6 Hz, 1H), 6.90 (d, J = 17.6 Hz, 1H), 4.70 (t, J = 6.4 Hz, 1H), 3.86 (d, J = 13.2 Hz, 1H), 3.73 (s, 3H), 3.61 (s, 3H), 3.55 (s, 1H), 2.88 (d, J = 13.2 Hz, 1H), 2.77-2.69 (m, 1H), 2.48 (d, J = 2.4 Hz, 6H), 2.25 (s, 3H), 1.00 (d, J = 6.4 Hz, 3H). |
| G | 170 | 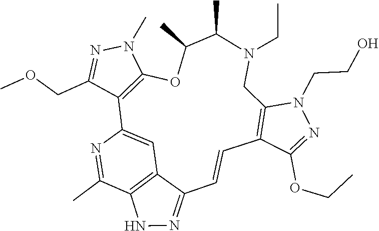 | 565.2 | 13.42-13.27 (m, 1H), 8.41 (s, 1H), 7.86-7.74 (m, 1H), 7.15 (d, J = 16.8 Hz, 1H), 5.00 (d, J = 11.2 Hz, 1H), 4.73- 4.64 (m, 1H), 4.29 (q, J = 7.2 Hz, 2H), 4.19- 4.10 (m, 1H), 4.07-3.97 (m, 2H), 3.90 (d, J = 14.0 Hz, 1H), 3.76 (s, 3H), 3.70 (d, J = 4.0 Hz, 2H), 3.35 (s, 4H), 3.28 (s, 3H), 3.01 (d, J = 2.8 Hz, 1H), 2.75 (s, 3H), 1.50-1.41 (m, 6H), 1.00 (d, J = 6.0 Hz, 3H), 0.86 (s, 3H). |
| G | 171 | 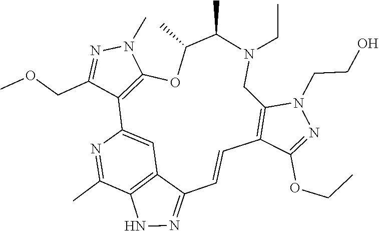 | 565.2 | ¹H NMR (400 MHz, DMSO-d₆) δ = 13.36 (d, J = 1.2 Hz, 1H), 8.47- 8.17 (m, 1H), 8.14-8.00 (m, 1H), 7.16 (d, J = 17.2 Hz, 1H), 5.10-4.92 (m, 1H), 4.89-4.79 (m, 1H), 4.72-4.55 (m, 1H), 4.28 (q, J = 6.8 Hz, 2H), 4.07 (s, 2H), 3.96-3.88 (m, 1H), 3.70 (s, 3H), 3.67 (d, J = 4.0 Hz, 1H), 3.33 (s, 6H), 3.27 (s, 3H), 2.75 (s, 3H), 1.42 (t, J = 7.2 Hz, 3H), 1.31 (d, J = 7.2 Hz, 3H), 1.16- 0.96 (m, 3H), 0.89 (t, J = 6.8 Hz, 3H). |
| — | 172 | 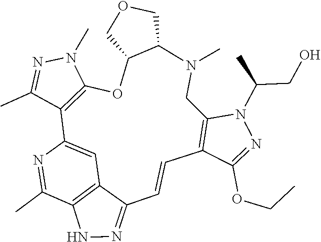 | 549.1 | ¹H NMR (400 MHz, MeOD-d₄) δ = 8.27 (s, 1H), 8.14 (d, J = 16.8 Hz, 1H), 7.22 (d, J = 16.8 Hz, 1H), 5.05 (br s, 1H), 4.52-4.41 (m, 1H), 4.41-4.27 (m, 3H), 4.09 (t, J = 7.6 Hz, 1H), 3.98 (br s, 2H), 3.88-3.80 (m, 1H), 3.73 (s, 3H), 3.71-3.65 (m, 3H), 3.65- 3.61 (m, 1H), 2.79 (s, 3H), 2.55 (d, J = 13.2 Hz, 6H), 1.48 (t, J = 7.2 Hz, 3H), 1.38 (d, J = 6.4 Hz, 3H). |
| — | 173 | 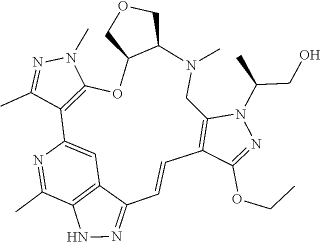 | 549.1 | ¹H NMR (400 MHz, MeOD-d₄) δ = 8.29 (s, 1H), 8.13 (d, J = 17.2 Hz, 1H), 7.21 (d, J = 16.8 Hz, 1H), 5.10 (br s, 1H), 4.50-4.41 (m, 1H), 4.40-4.31 (m, 2H), 4.30- 4.21 (m, 1H), 4.09 (t, J = 7.6 Hz, 1H), 3.99- 3.85 (m, 3H), 3.73 (s, 3H), 3.72-3.68 (m, 3H), 3.60 (br s, 1H), 2.78 (s, 3H), 2.56 (d, J = 12.0 Hz, 6H), 1.47 (t, J = 7.2 Hz, 3H), 1.36 (d, J = 6.4 Hz, 3H). |
| G | 174 | 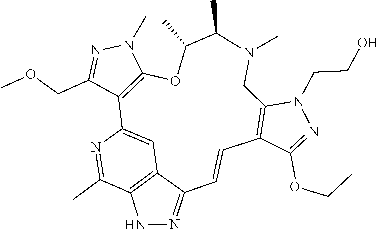 | 551.1 | ¹H NMR (400 MHz, METHANOL-d₄) δ = 8.65 (dd, J = 2.0, 5.2 Hz, 1H), 8.42-8.22 (m, 1H), 7.44 (d, J = 17.2 Hz, 1H), 5.08-5.02 (m, 1H), 4.99-4.94 (m, 1H), 4.46 (dd, J = 2.8, 5.2 Hz, 1H), 4.37 (q, J = 7.2 Hz, 2H), 4.21-4.11 (m, 3H), 4.00 ( d, J = 14.0 Hz, 1H), 3.89-3.84 (m, 2H), 3.81 (s, 3H), 3.51 (s, 3H), 3.09-2.96 (m, 1H), 2.90- 2.77 (m, 3H), 2.47 (s, 3H), 1.58-1.47 (m, 6H), 1.32 (d, J = 5.6 Hz, 3H). |
| G | 175 | 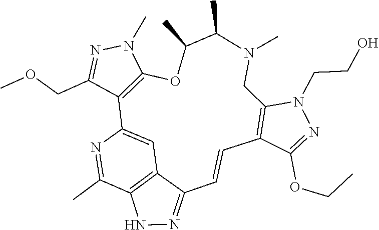 | 551.2 | ¹H NMR (400 MHz, METHANOL-d₄) δ = 8.52-8.31 (m, 1H), 8.13 (s, 1H), 7.32 (d, J = 17.2 Hz, 1H), 5.28-5.19 (m, 1H), 5.10 (d, J = 11.2 Hz, 1H), 4.77 (d, J = 11.2 Hz, 1H), 4.39-4.32 (m, 2H), 4.16-4.09 (m, 2H), 4.06 (s, 1H), 4.02- 3.93 (m, 1H), 3.84 (dt, J = 3.2, 5.2 Hz, 2H), 3.77 (s, 3H), 3.43 (s, 3H), 3.09-3.02 (m, 1H), 2.83 (s, 3H), 2.52-2.43 (m, 3H), 1.51 (t, J = 7.2 Hz, 3H), 1.39 (d, J = 6.8 Hz, 3H), 1.09 (d, J = 6.4 Hz, 3H). |
| C | 176 | 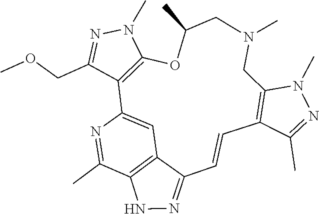 | 477.4 | ¹H NMR (400 MHz, DMSO-d₆) δ = 13.43 (s, 1H), 8.29-8.15 (m, 2H), 6.90 (d, J = 17.6 Hz, 1H), 4.99 (d, J = 10.8 Hz, 1H), 4.74 (t, J = 6.0 Hz, 1H), 4.64 (d, J = 10.8 Hz, 1H), 3.96 (d, J = 14.0 Hz, 1H), 3.78 (d, J = 14.0 Hz, 1H), 3.74 (s, 3H), 3.67 (s, 3H), 3.26 (s, 3H), 2.96-2.89 (m, 1H), 2.85-2.79 (m, 1H), 2.76 (s, 3H), 2.38 (s, 3H), 2.28 (s, 3H), 1.02 (d, J = 6.4 Hz, 3H). |
| A | 177 | 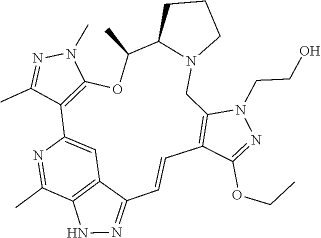 | 533.2 | ¹H NMR (400 MHz, MeOD-d₄) δ = 8.78- 8.56 (m, 1H), 8.36 (d, J = 16.8 Hz, 1H), 7.40- 7.25 (m, 1H), 4.52-4.42 (m, 1H), 4.39-4.30 (m, 2H), 4.25-4.15 (m, 2H), 4.14-4.04 (m, 1H), 3.98 (d, J = 14.0 Hz, 1H), 3.86-3.81 (m, 2H), 3.74 (s, 3H), 3.12-2.98 (m, 2H), 2.78 (s, 3H), 2.61 (s, 3H), 2.53-2.45 (m, 1H), 2.26-2.04 (m, 2H), 1.95-1.77 (m, 2H), 1.50 (t, J = 7.2 Hz, 3H), 1.37 (d, J = 3.2 Hz, 3H). |
| A | 178 | 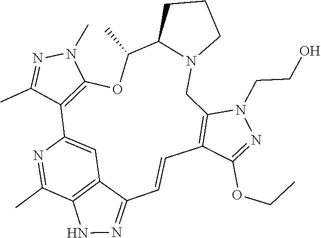 | 533.2 | ¹H NMR (400 MHz, MeOD-d₄) δ = 8.51- 8.27 (m, 1H), 7.95 (d, J = 16.8 Hz, 1H), 7.24 (d, J = 16.8 Hz, 1H), 4.60 (s, 1H), 4.38-4.33 (m, 2H), 4.30-4.22 (m, 2H), 4.14-4.05 (m, 2H), 3.91 (s, 1H), 3.89-3.83 (m, 3H), 3.72 (s, 3H), 2.80 (s, 3H), 2.57 (s, 3H), 2.54-2.47 (m, 1H), 2.22- 2.08 (m, 2H), 1.93- 1.73 (m, 2H), 1.50 (t, J = 7.2 Hz, 3H), 0.99 (s, 3H |
| A | 179 | 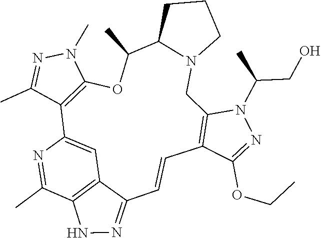 | 547.4 | ¹H NMR (400 MHz, MeOD-d₄) δ = 8.71- 8.59 (m, 1H), 8.30 (d, J = 16.8 Hz, 1H), 7.35- 7.25 (m, 1H), 4.55-4.44 (m, 2H), 4.40-4.33 (m, 2H), 4.31-4.23 (m, 1H), 3.93 (d, J = 14.0 Hz, 1H), 3.82-3.76 (m, 1H), 3.74 (s, 3H), 3.71-3.65 (m, 1H), 3.09-2.99 (m, 2H), 2.78 (s, 3H), 2.60 (s, 3H), 2.44-2.36 (m, 1H), 2.28-2.14 (m, 1H), 2.13-2.01 (m, 1H), 1.95- 1.84 (m, 1H), 1.82- 1.75 (m, 1H), 1.51-1.47 (m, 3H), 1.42 (d, J = 6.8 Hz, 3H), 1.39-1.31 (m, 3H). |
| A | 180 | 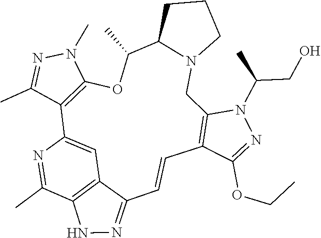 | 547.4 | ¹H NMR (400 MHz, MeOD) δ = 8.48-8.28 (m, 1H), 7.94-7.80 (m, 1H), 7.25-7.16 (m, 1H), 4.66-4.56 (m, 2H), 4.41- 4.32 (m, 3H), 3.92- 3.83 (m, 1H), 3.82-3.74 (m, 2H), 3.73 (s, 3H), 3.69-3.65 (m, 1H), 3.00- 2.85 (m, 1H), 2.80 (s, 3H), 2.57 (s, 3H), 2.50- 2.45 (m, 1H), 2.15 (d, J = 2.0 Hz, 1H), 2.20- 2.11 (m, 1H), 1.90-1.71 (m, 2H), 1.52-1.47 (m, 3H), 1.44 (d, J = 6.4 Hz, 3H), 1.35-1.25 (m, 1H), 1.00 (d, J = 2.0 Hz, 3H). |
| C | 181 | 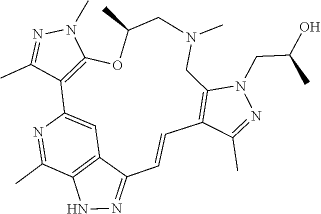 | 491.2 | ¹H NMR (400 MHz, MeOD-d₄) δ = 8.45 (d, J = 17.6 Hz, 1H), 8.24 (s, 1H), 6.99 (d, J = 17.6 Hz, 1H), 4.92-4.88 (m, 1H), 4.13-4.02 (m, 3H), 4.01-3.95 (m, 1H), 3.93-3.86 (m, 1H), 3.68 (s, 3H), 2.97-2.84 (m, 2H), 2.80 (s, 3H), 2.55 (s, 3H),2.49 (s, 3H), 2.38 (s, 3H), 1.25-1.18 (m, 3H), 1.08 (d, J = 6.4 Hz, 3H). |

### Biochemical Assays

Biochemical Assays for inhibition of wild-type and mutant EGFRs

The inhibitory activities against EGFR WT, EGFR A746-750 mutant, EGFR A746-750/C797S mutant, EGER L858R mutant, EGFR L858R/T790M mutant, EGFR L858R/T790M/C797S mutant, and EGFR (D770_N771insNPG) mutant were evaluated at Reaction Biology Corporation (See, www.reactionbiology.com) using HotSpot assay platform, a radiometric assay based on conventional filter-binding assays, that directly measures kinase catalytic activity toward a specific substrate (Anastassiadis T, et al., Comprehensive Assay of Kinase Catalytic Activity Reveals Features of Kinase Inhibitor Selectivity. Nat Biotechnol. 2011, 29:1039-45). Briefly, specific kinase/substrate pairs along with required cofactors were prepared in reaction buffer; 20 mM HEPES pH 7.5, 10 mM MgCn₂, 1 mM EGTA, 0.02% Brij35, 0.02 mg/ml BSA, 0.1 mM Na₃VO₄, 2 mM DTT, 1% DMSO. Compounds were delivered into the reaction, followed ~20 minutes later by addition of a mixture of ATP (Sigma, St. Louis MO) and ³³P ATP (Perkin Elmer, Waltham MA) to a final concentration of 10 M. Reactions were carried out at room temperature for 120 m, followed by spotting of the reactions onto P81 ion exchange filter paper (Whatman Inc., Piscataway, NJ). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

TABLE 1

| | | | EGFR | | | | |
|---|---|---|---|---|---|---|---|
| | | EGFR | (L858R / | EGFR | EGFR | EGFR | EGFR |
| | EGFR | (L858R/ | T790M/ | (D746- | (D746-750/ | (D770_ | WT |
| | (L858R) | T790M) | C797S/) | 750) | C797S) | N771insNPG) | IC₅₀, |
| Ex. | IC₅₀, nM | IC₅₀, nM | IC₅₀, nM | IC₅₀, nM | IC₅₀, nM | IC₅₀, nM | nM |
| Ex. 1 | 1.11 | 1.84 | 0.99 | 1.24 | 1.54 | 1.51 | 1.13 |
| Ex. 2 | | | 0.79 | 0.22 | 0.63 | 0.71 | |
| Ex. 3 | 1.50 | 1.33 | 0.85 | 1.27 | 1.56 | 3.80 | 1.83 |
| Ex. 4 | | | 1.36 | 0.59 | 1.44 | 3.07 | |
| Ex. 5 | 0.13 | 0.11 | 0.10 | 0.08 | 0.45 | 0.08 | 0.08 |
| Ex. 6 | 0.16 | 0.15 | 0.12 | 0.09 | 0.49 | 0.07 | 0.13 |
| Ex. 7 | 0.89 | 0.28 | 0.23 | 0.29 | 0.74 | 1.09 | 1.16 |
| Ex. 8 | 0.35 | 0.70 | 0.48 | 0.55 | 0.37 | 0.32 | 0.15 |
| Ex. 9 | 0.45 | 0.45 | 0.49 | 0.67 | 0.33 | 0.29 | 0.18 |
| Ex. 10 | 0.46 | 0.46 | 0.51 | 0.64 | 0.27 | 0.40 | 0.35 |
| Ex. 11 | 0.38 | 0.44 | 0.43 | 0.52 | 0.31 | 0.23 | 0.10 |
| Ex. 12 | 0.62 | 0.43 | 0.44 | 0.58 | 0.31 | 0.42 | 0.39 |
| Ex. 13 | 0.16 | 0.20 | 0.19 | 0.41 | 0.16 | 0.07 | 0.05 |
| Ex. 14 | 0.25 | 0.23 | 0.22 | 0.47 | 0.20 | 0.15 | 0.08 |
| Ex. 15 | 0.27 | 0.37 | 0.37 | 0.60 | 0.24 | 0.22 | 0.12 |
| Ex. 16 | 0.62 | 1.38 | 4.57 | 0.66 | 1.43 | 0.68 | 0.45 |
| Ex. 17 | 0.52 | 0.97 | 2.37 | 0.31 | 1.00 | 0.59 | 0.36 |
| Ex. 18 | 0.51 | 1.11 | 4.08 | 0.45 | 1.32 | 0.59 | 0.43 |
| Ex. 19 | 0.20 | 0.37 | 1.78 | 0.14 | 0.60 | 0.19 | 0.15 |
| Ex. 20 | 0.55 | 0.81 | 2.57 | 0.27 | 0.98 | 1.48 | 0.71 |
| Ex. 21 | 0.29 | 0.50 | 2.14 | 0.17 | 0.83 | 0.20 | 0.16 |
| Ex. 22 | 1.45 | 0.73 | 0.52 | 0.30 | 1.27 | 0.28 | 0.41 |
| Ex. 23 | 1.02 | 0.60 | 0.47 | 0.36 | 1.01 | 0.29 | 0.31 |
| Ex. 24 | 1.68 | 0.94 | 0.57 | 0.51 | 1.08 | 0.62 | 0.50 |
| Ex. 25 | 1.88 | 1.39 | 0.51 | 0.51 | 1.02 | 1.23 | 0.55 |
| Ex. 26 | 2.39 | 1.44 | 0.93 | 0.73 | 1.97 | 0.90 | 0.80 |
| Ex. 27 | 0.89 | 0.51 | 0.45 | 0.28 | 0.90 | 0.27 | 0.31 |
| Ex. 28 | 1.99 | 0.46 | 0.41 | 0.37 | 0.85 | 1.23 | 1.03 |
| Ex. 29 | 0.72 | 0.30 | 0.30 | 0.20 | 0.65 | 0.29 | 0.22 |
| Ex. 30 | | | 0.19 | 0.15 | 0.49 | 0.15 | |
| Ex. 31 | | | 0.29 | 0.27 | 0.78 | 2.23 | |
| Ex. 32 | | | 0.77 | 0.33 | 1.09 | 0.63 | |
| Ex. 33 | | | 0.31 | 0.15 | 0.66 | 0.17 | |
| Ex. 34 | | | 0.28 | 0.12 | 0.31 | 0.11 | |
| Ex. 35 | | | 0.41 | 0.22 | 0.66 | 0.24 | |
| Ex. 36 | | | 0.13 | 0.10 | 0.29 | 0.14 | |
| Ex. 37 | | | 0.71 | 0.31 | 0.79 | 1.62 | |
| Ex. 38 | | | 0.25 | 0.24 | 0.54 | 1.90 | |
| Ex. 39 | | | 0.50 | 0.32 | 0.85 | 0.48 | |
| Ex. 40 | | | 0.45 | 0.35 | 0.97 | 1.63 | |
| Ex. 41 | | | 0.23 | 0.08 | 0.23 | 0.08 | |
| Ex. 42 | | | 0.56 | 0.29 | 0.83 | 0.35 | |
| Ex. 43 | | | 0.16 | 0.09 | 0.23 | 0.18 | |
| Ex. 44 | | | 0.57 | 0.26 | 0.62 | 0.17 | |
| Ex. 45 | | | 0.53 | 0.28 | 0.95 | 0.69 | |
| Ex. 46 | | | 0.23 | 0.15 | 0.40 | 0.36 | |
| Ex. 47 | 0.20 | 0.32 | 0.28 | 0.43 | 0.27 | 0.20 | 0.18 |
| Ex. 48 | | | 0.37 | 0.18 | 0.63 | 0.23 | |
| Ex. 49 | | | 0.21 | 0.12 | 0.34 | 0.19 | |
| Ex. 50 | | | 0.39 | 0.19 | 0.68 | 0.21 | |
| Ex. 51 | | | 0.37 | 0.31 | 0.83 | 1.11 | |
| Ex. 52 | | | 0.46 | 0.21 | 0.71 | 0.25 | |
| Ex. 53 | | | 0.67 | 0.37 | 1.12 | 0.45 | |
| Ex. 54 | 0.32 | 1.04 | 0.65 | 0.67 | 0.48 | 0.55 | 0.38 |
| Ex. 55 | | | 0.53 | 0.25 | 0.72 | 1.38 | |
| Ex. 56 | | | 0.98 | 0.45 | 1.11 | 0.67 | |
| Ex. 57 | | | 0.86 | 0.80 | 1.38 | 4.83 | |
| Ex. 58 | | | 1.01 | 0.55 | 1.38 | 0.72 | |
| Ex. 59 | | | 0.99 | 0.37 | 1.33 | 1.13 | |
| Ex. 60 | | | 0.86 | 0.54 | 1.72 | 0.78 | |
| Ex. 61 | | | 0.91 | 0.43 | 1.05 | 0.70 | |
| Ex. 62 | | | 0.74 | 0.42 | 1.58 | 0.42 | |
| Ex. 63 | | | 0.60 | 0.36 | 1.09 | 0.39 | |
| Ex. 64 | | | 0.82 | 0.41 | 1.20 | 0.42 | |
| Ex. 65 | | | 1.58 | 0.61 | 1.61 | 0.99 | |
| Ex. 66 | | | 0.54 | 0.36 | 1.04 | 0.42 | |
| Ex. 67 | | | 1.08 | 0.56 | 1.65 | 0.69 | |
| Ex. 68 | | | 0.44 | 0.33 | 1.18 | 0.95 | |
| Ex. 69 | | | 1.93 | 1.87 | 3.68 | 15.27 | |
| Ex. 70 | | | 0.31 | 0.16 | 0.69 | 0.41 | |
| Ex. 74 | | | 1.50 | 0.55 | 1.83 | 0.66 | |
| Ex. 110 | 0.36 | 0.50 | 0.25 | 0.46 | 0.56 | 0.18 | 0.09 |
| Ex. 180 | 0.15 | 0.16 | 0.13 | 0.21 | 0.21 | 0.10 | 0.05 |

### Cellular Assays

### Cellular EGFR Phosphorylation Assays

EGFR phosphorylation assays were conducted in NSCLC cells with EGFR activation mutations. PC-9 cells (purchased from Aldrich-Sigma) carry a Glu746-Ala750 deletion (d746-750) mutation in exon 19 of the EGFR gene and NCI-H1975 cells (purchased from ATCC) carry both L858R activation mutation and T790M gatekeeper mutation in the EGFR gene. A431 cells (purchased from ATCC) with amplification of wildtype EGFR, derived from epidermoid carcinoma, were used to measure the activity against wildtype EGFR. All cells were seeded in 96-well plates. For PC-9 and NCI-H1975 cells, 50 thousand cells were seeded per well. For A431 cells, 30 thousand cells were seeded per well. The next day, cells were exposed to compound for 4 hours at 37° C. and 5% CO₂. Compounds were diluted in DMSO and added to cells in a 9-dose, 3-fold dilution, titration curve using TECAN d300e liquid dispenser. For A431 cells, 40 ng/mL of EGF was added to the media for the final 10 minutes of incubation. EGFR phosphorylation was measured using phospho-EGFR (Try1068) LANCE Ultra TR-FRET Detection Kit (TRF4015M) from PerkinElmer according to manufacturer's instructions. In brief, cells were lysed with 40 μL of lysis buffer plus 1× Halt protease and phosphatase inhibitor cocktail (Thermo 78441) and by incubation at room temperature for 1 h on a microplate shaker (350 rpm). 15 μ L of cell lysate was transferred to 384-well white plate and 5 μL of Europium- and ULight-labeled antibodies diluted in 1× detection buffer was added to each well. After 4-6 hours of incubation at room temperature, plates were then read on a TECAN Sparks multimode microplate reader. Data was analyzed using GraphPad Prism 9 (GraphPad Software, San Diego, CA) to obtain IC₅₀ values.

TABLE 2

| | PC-9 | H1975 | A431 |
|---|---|---|---|
| Ex. | pEGFR IC₅₀, nM | pEGFR IC₅₀, nM | pEGFR IC₅₀, nM |
| Ex. 1 | 3.09 | 3.01 | 19.08 |
| Ex. 2 | 1.35 | 1.34 | 9.27 |
| Ex. 3 | 14.68 | 6.43 | 36.47 |
| Ex. 4 | 789.30 | 17.67 | 442.30 |
| Ex. 5 | 6.40 | 3.99 | 29.17 |
| Ex. 6 | 6.85 | 10.92 | 42.68 |
| Ex. 7 | 24.30 | 14.23 | 70.91 |
| Ex. 8 | 1.05 | 1.54 | 8.10 |
| Ex. 9 | 3.48 | 4.19 | 19.79 |
| Ex. 10 | 10.81 | 4.82 | 51.89 |
| Ex. 11 | 2.86 | 5.25 | 16.14 |
| Ex. 12 | 6.38 | 3.64 | 40.70 |
| Ex. 13 | 1.00 | 2.09 | 12.01 |
| Ex. 14 | 11.76 | 7.52 | 44.67 |
| Ex. 15 | 1.05 | 1.86 | 7.48 |
| Ex. 16 | 4.37 | 5.22 | 19.77 |
| Ex. 17 | 1.85 | 2.63 | 14.33 |
| Ex. 18 | 3.24 | 8.91 | 15.59 |
| Ex. 19 | 2.54 | 2.85 | 10.00 |
| Ex. 20 | 11.76 | 8.57 | 60.98 |
| Ex. 21 | 1.69 | 2.32 | 15.77 |
| Ex. 22 | 5.67 | 6.81 | 67.49 |
| Ex. 23 | 3.42 | 5.84 | 43.10 |
| Ex. 24 | 1.50 | 1.60 | 22.43 |
| Ex. 25 | 1.24 | 1.93 | 17.86 |
| Ex. 26 | 1.60 | 3.40 | 12.17 |
| Ex. 27 | 0.91 | 1.85 | 12.50 |
| Ex. 28 | 14.39 | 3.00 | 52.83 |
| Ex. 29 | 3.63 | 0.93 | 13.69 |
| Ex. 30 | 3.64 | 3.31 | 14.03 |
| Ex. 31 | 65.09 | 41.60 | 118.80 |
| Ex. 32 | 1.08 | 1.48 | 9.53 |
| Ex. 33 | 1.50 | 2.38 | 17.58 |
| Ex. 35 | 1.56 | 1.25 | 15.13 |
| Ex. 38 | 18.82 | 3.11 | 47.53 |
| Ex. 39 | 2.11 | 2.18 | 22.75 |
| Ex. 40 | 11.40 | 1.60 | 14.67 |
| Ex. 44 | 1.01 | 1.92 | 9.94 |
| Ex. 45 | 2.15 | 1.24 | 12.16 |
| Ex. 47 | 1.13 | 1.06 | 15.22 |
| Ex. 48 | 3.14 | 6.12 | 36.97 |
| Ex. 50 | 1.07 | 3.43 | 19.25 |
| Ex. 53 | 2.57 | 8.74 | 21.02 |
| Ex. 54 | 0.67 | 0.94 | 10.66 |
| Ex. 56 | 0.82 | 1.26 | 8.41 |
| Ex. 57 | 11.27 | 10.52 | 26.06 |
| Ex. 58 | 1.11 | 2.34 | 21.50 |
| Ex. 59 | 1.96 | 1.43 | 5.29 |
| Ex. 60 | 2.39 | 2.49 | 29.04 |
| Ex. 61 | 1.68 | 2.05 | 20.96 |
| Ex. 62 | 1.13 | 1.54 | 13.20 |
| Ex. 63 | 2.38 | 3.31 | 57.34 |
| Ex. 64 | 2.74 | 2.79 | 29.58 |
| Ex. 65 | 1.11 | 1.56 | 15.84 |
| Ex. 67 | 2.98 | 5.54 | 15.99 |
| Ex. 68 | 6.22 | 6.39 | 20.97 |
| Ex. 69 | 75.36 | 261.80 | 160.20 |
| Ex. 73 | 72.63 | | 563.80 |
| Ex. 74 | 1.54 | 5.16 | 8.81 |
| Ex. 75 | 15.02 | 7.41 | 53.67 |
| Ex. 82 | 2.35 | 14.96 | 24.71 |
| Ex. 86 | 1.09 | 1.57 | 7.67 |
| Ex. 87 | 0.97 | 1.82 | 5.88 |
| Ex. 88 | 1.62 | 2.17 | 10.98 |
| Ex. 89 | 16.13 | 48.06 | 34.27 |
| Ex. 90 | 1.26 | 1.10 | 8.44 |
| Ex. 91 | 69.50 | 21.33 | 34.78 |
| Ex. 92 | 12.57 | 14.05 | 33.40 |
| Ex. 93 | 2.05 | 3.54 | 19.63 |
| Ex. 94 | 2.31 | 1.55 | 17.78 |
| Ex. 95 | 3.35 | 1.76 | 17.70 |
| Ex. 97 | 1.08 | 1.62 | 10.96 |
| Ex. 98 | 3.62 | 1.90 | 16.81 |
| Ex. 101 | 4.17 | 2.68 | 34.67 |
| Ex. 103 | 9.78 | 3.44 | 13.41 |
| Ex. 104 | 39.76 | 50.48 | 145.60 |
| Ex. 105 | 318.90 | 273.80 | 364.50 |
| Ex. 106 | 9.00 | 3.03 | 10.21 |
| Ex. 107 | 4.41 | 7.07 | 5.13 |
| Ex. 109 | 2.91 | 1.79 | 3.52 |
| Ex. 110 | 1.12 | 1.76 | 1.42 |
| Ex. 111 | 1.06 | 2.08 | 1.46 |
| Ex. 113 | 1.98 | 1.58 | 11.07 |
| Ex. 114 | 3.56 | 5.67 | 22.25 |
| Ex. 147 | | | 11.34 |
| Ex. 148 | 1.94 | 2.90 | 40.46 |
| Ex. 151 | 2.04 | 3.24 | 26.64 |
| Ex. 177 | 1.01 | 1.38 | 8.86 |
| Ex. 178 | 0.81 | 1.29 | 12.47 |
| Ex. 179 | 0.69 | 1.38 | 8.64 |
| Ex. 180 | 0.64 | 1.54 | 6.08 |

### 5-Day CellTiter Glo (CTG) Cell Proliferation Assays (PC9, H1975, H827)

Cell proliferation assays were carried out in NSCLC cells with EGFR activation mutations. Both PC-9 cells and HCC827 cells (purchased from DSMZ) carry a Glu746-Ala750 deletion (d746-750) mutation in exon 19 of the EGFR gene and NCI-H1975 cells carry both L858R activation mutation and T790M gatekeeper mutation in the EGFR gene. One thousand PC-9, NCI-H1975 or HCC827 cells/100 μL per well were seeded in 96-well black plate with transparent bottom and then treated with indicated compounds in a 9-dose dilution series dispensed using TECAN d300e liquid dispenser. Cells were incubated with compounds for 120 hours at 37° C. and 5% CO₂. Cell proliferation was measured using CellTiter-Glo 2.0 luciferase-based ATP detection assay (Promega, Madison, WI) at 50 ML per well following the manufacturer's protocol. Plates were then read on a TECAN Sparks multimode microplate reader. Data was analyzed using GraphPad Prism 9 (GraphPad Software, San Diego, CA) to obtain IC₅₀ values.

TABLE 3

| | PC-9 | H1975 | HCC827 |
|---|---|---|---|
| Ex. | IC₅₀, nM | IC₅₀, nM | IC₅₀, nM |
| Ex. 1 | 4.66 | 28.62 | 0.68 |
| Ex. 2 | 1.81 | 8.82 | 0.39 |
| Ex. 3 | 55.41 | 29.63 | 4.42 |
| Ex. 4 | 114.80 | 74.89 | 6.03 |
| Ex. 5 | 33.54 | 4.88 | 0.99 |
| Ex. 6 | 27.63 | 27.99 | 1.98 |
| Ex. 7 | 59.70 | 47.42 | 5.17 |
| Ex. 8 | 0.36 | 0.92 | 0.11 |
| Ex. 9 | 12.06 | 3.22 | 0.39 |
| Ex. 11 | 3.65 | 3.14 | 0.25 |
| Ex. 12 | 13.77 | 22.17 | 2.25 |
| Ex. 13 | 0.38 | 4.27 | 0.26 |
| Ex. 14 | 54.50 | 109.80 | 2.87 |
| Ex. 15 | 0.33 | 0.76 | 0.18 |
| Ex. 16 | 5.56 | 27.12 | 0.88 |
| Ex. 17 | 2.01 | 17.51 | 0.37 |
| Ex. 18 | 2.14 | 19.51 | 0.53 |
| Ex. 19 | 2.74 | 1.51 | 0.28 |
| Ex. 20 | 25.16 | 95.97 | 2.89 |
| Ex. 21 | 0.63 | 5.72 | 0.26 |
| Ex. 22 | 7.12 | 19.84 | 0.87 |
| Ex. 23 | 2.91 | 10.04 | 0.41 |
| Ex. 24 | 0.47 | 1.26 | 0.27 |
| Ex. 25 | 2.97 | 10.34 | 0.46 |
| Ex. 26 | 0.44 | 1.11 | 0.27 |
| Ex. 27 | 0.45 | 0.97 | 0.26 |
| Ex. 28 | 16.84 | 33.00 | 2.33 |
| Ex. 29 | 11.44 | 1.04 | 1.12 |
| Ex. 30 | 4.44 | 1.96 | 0.50 |
| Ex. 31 | 225.60 | 357.50 | 11.57 |
| Ex. 32 | 0.54 | 1.39 | 0.25 |
| Ex. 33 | 0.58 | 1.10 | 0.44 |
| Ex. 34 | 4.22 | 1.75 | 0.39 |
| Ex. 35 | 2.56 | 3.05 | 0.51 |
| Ex. 38 | 48.79 | 27.11 | 7.90 |
| Ex. 39 | 6.57 | 26.44 | 0.63 |
| Ex. 44 | 0.84 | 1.05 | 0.29 |
| Ex. 45 | 3.31 | 1.09 | 1.12 |
| Ex. 47 | 1.80 | 1.77 | 0.57 |
| Ex. 48 | 17.83 | 55.65 | 2.08 |
| Ex. 50 | 0.51 | 5.55 | 0.41 |
| Ex. 53 | 2.94 | 15.74 | 1.10 |
| Ex. 54 | 0.78 | 0.99 | 0.41 |
| Ex. 56 | 0.32 | 0.92 | 0.28 |
| Ex. 57 | 21.91 | 68.20 | 8.59 |
| Ex. 58 | 2.44 | 14.71 | 0.75 |
| Ex. 59 | 3.21 | 15.68 | 0.94 |
| Ex. 60 | 5.05 | 57.53 | 1.77 |
| Ex. 61 | 3.55 | 74.69 | 0.66 |
| Ex. 62 | 1.08 | 16.91 | 0.44 |
| Ex. 63 | 13.80 | 9.62 | 2.70 |
| Ex. 64 | 5.63 | 26.60 | 1.02 |
| Ex. 65 | 1.60 | 5.36 | 0.63 |
| Ex. 67 | 1.81 | 17.57 | 1.13 |
| Ex. 68 | 15.42 | 226.60 | 2.49 |
| Ex. 69 | 92.01 | 307.60 | 41.88 |
| Ex. 73 | 23.73 | 5.53 | 0.24 |
| Ex. 74 | 0.68 | 79.90 | 0.72 |
| Ex. 75 | 43.15 | 74.03 | 10.57 |
| Ex. 82 | 1.54 | 206.75 | 0.85 |
| Ex. 86 | 0.68 | 1.11 | 0.30 |
| Ex. 87 | 0.36 | 0.57 | 0.23 |
| Ex. 88 | 2.52 | 5.09 | 0.39 |
| Ex. 89 | 35.29 | 125.00 | 11.04 |
| Ex. 90 | 2.37 | 0.97 | 0.58 |
| Ex. 91 | 63.00 | 57.04 | 16.79 |
| Ex. 92 | 43.37 | 68.51 | 5.37 |
| Ex. 93 | 1.98 | 2.00 | 0.51 |
| Ex. 94 | 3.52 | 1.76 | 0.71 |
| Ex. 95 | 5.19 | 2.86 | 1.15 |
| Ex. 96 | 25.46 | 27.17 | 2.97 |
| Ex. 97 | 1.39 | 1.77 | 0.20 |
| Ex. 98 | 5.71 | 3.34 | 1.35 |
| Ex. 99 | 350.80 | 338.50 | 38.11 |
| Ex. 101 | 8.86 | 3.60 | 1.05 |
| Ex. 102 | 52.79 | 57.00 | 25.27 |
| Ex. 103 | 9.94 | 11.22 | 2.13 |
| Ex. 104 | 58.95 | 3000.00 | 13.87 |
| Ex. 105 | 209.10 | 1000.00 | 52.12 |
| Ex. 106 | 14.64 | 7.92 | 2.18 |
| Ex. 107 | 10.62 | 55.25 | 2.12 |
| Ex. 109 | 5.53 | 2.65 | 0.90 |
| Ex. 110 | 0.47 | 1.07 | 0.31 |
| Ex. 111 | 1.52 | 3.12 | 0.43 |
| Ex. 113 | 0.68 | 1.40 | 0.45 |
| Ex. 114 | 4.87 | 27.05 | 1.66 |
| Ex. 115 | 3.35 | 6.13 | 1.47 |
| Ex. 127 | 1.26 | 1.92 | 0.37 |
| Ex. 128 | 17.04 | 56.78 | 3.32 |
| Ex. 130 | 2.32 | 175.90 | 0.75 |
| Ex. 131 | 353.20 | 671.30 | 60.48 |
| Ex. 132a | 1.12 | 0.77 | 0.41 |
| Ex. 132b | 0.54 | 0.70 | 0.29 |
| Ex. 133 | 34.25 | 40.54 | 8.87 |
| Ex. 134 | 1000 | 1000 | 1000 |
| Ex. 135 | 115.5 | 1000 | 73.78 |
| Ex. 136 | 66.99 | 266.0 | 34.4 |
| Ex. 137 | 113.3 | 1000 | 90.73 |
| Ex. 138 | 1000 | 1000 | 1000 |
| Ex. 139 | 4.35 | 428.9 | 0.82 |
| Ex. 140 | 29.9 | 1000 | 6.10 |
| Ex. 141 | 1000 | 1000 | 1000 |
| Ex. 143 | 21.56 | 518.8 | 9.09 |
| Ex. 145 | 0.37 | 0.58 | 0.24 |
| Ex. 147 | 0.59 | 20.38 | 0.34 |
| Ex. 148 | 0.76 | 1.76 | 0.29 |
| Ex. 151 | 1.55 | 4.89 | 0.42 |
| Ex. 177 | 1.43 | 2.60 | 0.39 |
| Ex. 178 | 1.02 | 0.93 | 0.39 |
| Ex. 179 | 0.78 | 1.41 | 0.23 |
| Ex. 180 | 0.70 | 0.95 | 0.34 |

### 3-DAY CellTiter Glo (CTG) Cell Proliferation Assays (BaF3 Cell Lines)

Cell proliferation assays were carried out in BaF3 cells engineered with EGFR mutations. BaF3 cells with engineered EGFR L858R/T790M/C797S, EGFR L858R/C797S, or EGFR d746-750/T790M transgene, one thousand cells/50 μL per well were seeded in white 384-well plate, followed by treatment with indicated compounds in a 9-dose dilution series using TECAN d300e liquid dispenser. Cells were incubated with compounds for 72 hours at 37° C. and 5% CO₂. Cell proliferation was measured using CellTiter-Gbo 2.0 luciferase-based ATP detection assay (Promega, Madison, WI) at 18 μL per well following the manufacturer's protocol. Plates were then read on a TECAN Sparks multimode microplate reader. Data was analyzed using GraphPad Prism 9 (GraphiPad Software, San Diego, CA) to obtain IC₅₀ values.

TABLE 4

| | EGFR | EGFR | EGFR |
|---|---|---|---|
| | d746-750/T790M | L858R/C797S | L858R/T790M/C797S |
| Ex. | IC₅₀, nM | IC₅₀, nM | IC₅₀, nM |
| Ex. 1 | 1.70 | 0.41 | 1.57 |
| Ex. 2 | 0.71 | 0.13 | 0.90 |
| Ex. 3 | 4.51 | 1.30 | 1.81 |
| Ex. 4 | | 2.60 | 2.81 |
| Ex. 8 | 0.44 | 0.10 | 0.81 |
| Ex. 9 | | 0.19 | 0.93 |
| Ex. 10 | 0.69 | | 1.89 |
| Ex. 11 | | 0.16 | 0.83 |
| Ex. 12 | | 0.78 | 1.36 |
| Ex. 13 | 1.46 | 0.10 | 1.05 |
| Ex. 14 | 22.29 | 1.24 | 5.24 |
| Ex. 15 | 0.58 | 0.06 | 0.79 |
| Ex. 16 | 0.66 | 0.34 | 1.93 |
| Ex. 17 | 0.34 | 0.22 | 1.14 |
| Ex. 18 | 1.44 | 0.29 | 2.46 |
| Ex. 19 | | 0.16 | 0.79 |
| Ex. 20 | | 3.05 | 5.29 |
| Ex. 21 | | 0.14 | 0.90 |
| Ex. 22 | 5.77 | 0.56 | 4.39 |
| Ex. 23 | 3.77 | 0.43 | 2.69 |
| Ex. 24 | 0.41 | 0.11 | 0.78 |
| Ex. 25 | 0.63 | 0.13 | 0.85 |
| Ex. 26 | 0.66 | 0.11 | 1.16 |
| Ex. 27 | 0.55 | 0.07 | 0.73 |
| Ex. 28 | 1.16 | 2.47 | 1.71 |
| Ex. 29 | 0.90 | 0.17 | 0.74 |
| Ex. 30 | 0.65 | 0.14 | 1.02 |
| Ex. 31 | | 3.72 | 25.89 |
| Ex. 32 | 0.48 | 0.07 | 0.87 |
| Ex. 33 | 1.07 | 0.11 | 1.60 |
| Ex. 35 | 0.46 | 0.14 | 0.89 |
| Ex. 36 | 2.13 | 0.47 | 1.97 |
| Ex. 38 | 22.08 | 6.50 | 4.01 |
| Ex. 39 | 6.08 | 0.27 | 1.87 |
| Ex. 40 | 3.34 | 0.28 | 0.92 |
| Ex. 41 | 13.79 | 0.16 | 3.74 |
| Ex. 44 | 1.18 | 0.05 | 0.57 |
| Ex. 45 | 1.04 | 0.12 | 0.47 |
| Ex. 47 | 1.50 | 0.07 | 0.62 |
| Ex. 48 | 17.07 | 0.16 | 1.21 |
| Ex. 50 | 2.99 | 0.12 | 1.14 |
| Ex. 53 | 4.31 | 0.18 | 1.57 |
| Ex. 54 | 0.55 | 0.06 | 0.55 |
| Ex. 56 | 0.48 | 0.05 | 0.61 |
| Ex. 57 | 61.80 | 0.69 | 1.77 |
| Ex. 59 | 3.62 | 0.18 | 0.91 |
| Ex. 73 | | | 35.76 |
| Ex. 74 | 6.61 | 0.04 | 1.34 |
| Ex. 75 | 203.10 | 0.22 | 2.94 |
| Ex. 82 | 66.29 | 0.12 | 4.40 |
| Ex. 86 | 0.69 | 0.04 | 0.54 |
| Ex. 88 | 1.56 | 0.09 | 0.86 |
| Ex. 89 | 43.34 | 3.84 | 20.75 |
| Ex. 90 | 0.61 | 0.06 | 0.48 |
| Ex. 91 | 45.74 | 9.29 | 17.25 |
| Ex. 92 | 29.74 | 0.74 | 4.12 |
| Ex. 93 | 1.80 | 0.13 | 1.65 |
| Ex. 94 | 1.23 | 0.09 | 0.93 |
| Ex. 95 | 2.14 | 0.13 | 0.74 |
| Ex. 97 | 0.40 | 0.04 | 0.43 |
| Ex. 98 | 1.01 | 0.24 | 0.74 |
| Ex. 101 | 4.36 | 0.20 | 1.37 |
| Ex. 102 | 204.00 | 3.16 | 12.20 |
| Ex. 103 | 13.57 | 0.56 | 1.87 |
| Ex. 104 | 63.89 | 6.89 | 36.16 |
| Ex. 105 | 699.50 | 15.47 | 147.10 |
| Ex. 106 | 4.75 | 1.37 | 3.41 |
| Ex. 107 | 4.00 | 0.49 | 3.55 |
| Ex. 109 | 1.17 | 0.24 | 1.17 |
| Ex. 110 | 0.61 | 0.07 | 0.71 |
| Ex. 111 | 0.78 | 0.11 | 1.11 |
| Ex. 112 | 396.00 | 29.50 | 97.66 |
| Ex. 113 | 1.18 | 0.11 | 0.91 |
| Ex. 114 | 3.72 | 0.42 | 1.84 |
| Ex. 115 | 3.05 | 9.04 | 31.90 |
| Ex. 177 | 0.88 | 0.07 | 0.97 |
| Ex. 178 | 0.67 | 0.06 | 0.59 |
| Ex. 179 | 0.88 | 0.10 | 1.47 |
| Ex. 180 | 0.57 | 0.06 | 0.69 |

What is claimed is:

1. A compound of the formula I 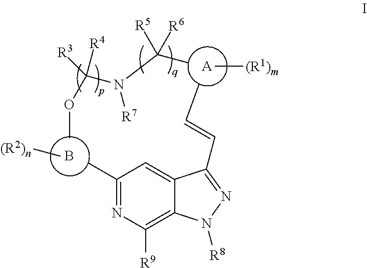
wherein
ring A and ring B are each independently a 5-membered heteroarylene;
each R¹ and R², when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5-to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{e}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{e}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NRS(O)₂NR^{c}R^{d}, —C(O)R, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R³, R⁴, R⁵, and R⁶ is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5-to 10-membered heteroaryl, —OR′, —OC(O)R^{a}, —OC(O)NR^{a}R^{b}, —OS(O)R^{a}, —OS(O)₂R^{a}, —SR^{a}, —S(O)R^{a}, —S(O)₂R^{c}, —S(O)NR^{a}R^{e}, —S(O)₂NR^{a}R^{e}, —OS(O)NR^{a}R^{b}, —OS(O)₂NR^{a}R^{e}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NRS(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}—P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or two of R³, R⁴, R⁵, and R⁶, taken together with the carbon or carbons to which they are attached, combine to form C₃-C₆ cycloalkyl or 3- to 7-membered heterocycloalkyl, wherein each hydrogen atom in C₃-C₆ cycloalkyl and 3- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{c}S(O)₂R^{f}, —NR^{e}S(O)NR^{e}R^{f}, —NR^{c}S(O)₂NR^{e}R^{f}, —C(O)R^{c}, —C(O)OR^{c}, —C(O)NR^{e}R^{f}, —PR^{c}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁷ is H, deuterium, C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, 5- to 10-membered heteroaryl, —C(O)R^{e}, or —C(O)NR^{c}R^{d}, wherein each hydrogen atom in C₁-C₆ alkyl, C₂-C₆ alkenyl, C₂-C₆ alkynyl, C₃-C₆ cycloalkyl, 4- to 7-membered heterocycloalkyl, C₆-C₁₀ aryl, and 5- to 10-membered heteroaryl is independently optionally substituted by —OR^{c}, —OC(O)R^{e}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{a}), —NR^{c}C(O)OR^{d}, —NR^{c}C(O)NR^{c}R^{d}, —NR^{f}C(═NR^{d})NR^{c}R^{d}, —NR^{c}S(O)R^{d}, —NRCS(O)₂R^{d}, —NR^{e}S(O)NR^{c}R^{d}, —NR^{c}S(O)₂NR^{c}R^{d}, —C(O)R^{e}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{e}, —P(O)₂OR^{e}, —CN, or —NO₂; or R⁷ and one of R³, R⁴, R⁵, or R⁶, taken together with the atoms to which each is attached, combine to form a 4- to 7-membered heterocycloalkyl, wherein each hydrogen atom in 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, 0173SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{c}, —CN, or —NO₂;
R⁸ is H, —S(O)₂R^{c}, —S(O)₂NR^{c}R^{d}, —C(O)R^{c}, —C(O)OR^{c}, —C(O)NR^{c}R^{d}, —C(═NR^{d})NR^{c}R^{d}, —P(O)₂R^{c}R^{d}, —P(O)₂NR^{c}R^{d}, or —P(O)₂OR¹;
R⁹ is H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR—, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl is independently optionally substituted by deuterium, halogen, C₁-C₆ alkyl, C₁-C₆ haloalkyl, —OR^{e}, —OC(O)R^{e}, —OC(O)NR^{c}R^{d}, —OC(═NR^{d})NR^{c}R^{d}, —OS(O)R^{c}, —OS(O)₂R^{c}, —OS(O)NR^{e}R^{d}, —OS(O)₂NR^{c}R^{d}, —SR^{e}, —S(O)R^{e}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{d}, —P(O)₂OR^{e}, —CN, or —NO₂;
each R^{a}, R^{b}, R^{e}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl; or R^{a} and R^{b} or R^{e} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3-to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl is independently optionally substituted by deuterium, halogen, C₁-C₆ alkyl, C₁-C₆haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, or 3;
n is 0, 1, 2, or 3;
p is 1, 2, 3, or 4; and
q is 1, 2, or 3;
or a pharmaceutically acceptable salt, solvate, hydrate, or cocrystal thereof.

2. The compound of claim 1, having the formula II 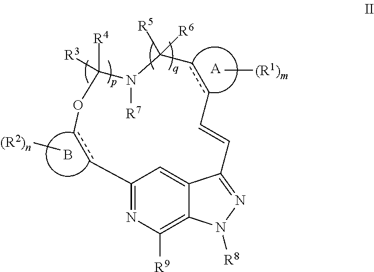
wherein each “ [symbol] ” is independently a carbon-carbon single bond or a carbon-carbon double bond;
or a pharmaceutically acceptable salt, solvate, hydrate, or cocrystal thereof.

3. The compound of claim 1 or 2, having the formula III 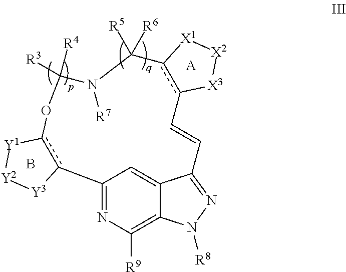
wherein
each “ [symbol] ” is independently a carbon-carbon single bond or a carbon-carbon double bond:
X¹, X², and X³ are each independently —O—, —S—, ═C(H)—, ═C(R¹)—, —N(H)—, —N(R¹)— or ═N—, provided that at least one of X¹, X², and X³ is not ═C(H)—, or ═C(R¹)—; and/or
Y¹, Y², and Y³ are each independently —O—, —S—, ═C(H)—, ═C(R²)—, —N(H)—, —N(R²)— or ═N—, provided that at least one of Y¹, Y², and Y³ is not ═C(H)—, or ═C(R²)—;
or a pharmaceutically acceptable salt, solvate, hydrate, or cocrystal thereof.

4. The compound of claim 3, having the formula IV 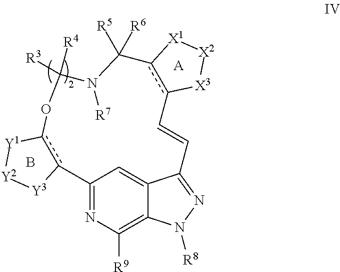
or a pharmaceutically acceptable salt, solvate, hydrate, or cocrystal thereof.

5. The compound of claim 3 or 4, wherein
X² is ═N— or —N(R¹)—, X¹ and X³ are each independently —O—, —S—, ═C(H)—, ═C(R¹)—, —N(H)—, or —N(R¹)—; and/or
Y² is ═N—, Y¹ and Y³ are each independently —O—, —S—, ═C(H)—, ═C(R²)—, —N(H)—, or —N(R²)—;
or a pharmaceutically acceptable salt, solvate, hydrate, or cocrystal thereof.

6. The compound of any one of claims 1 to 4, wherein:
ring A is selected from the group consisting of 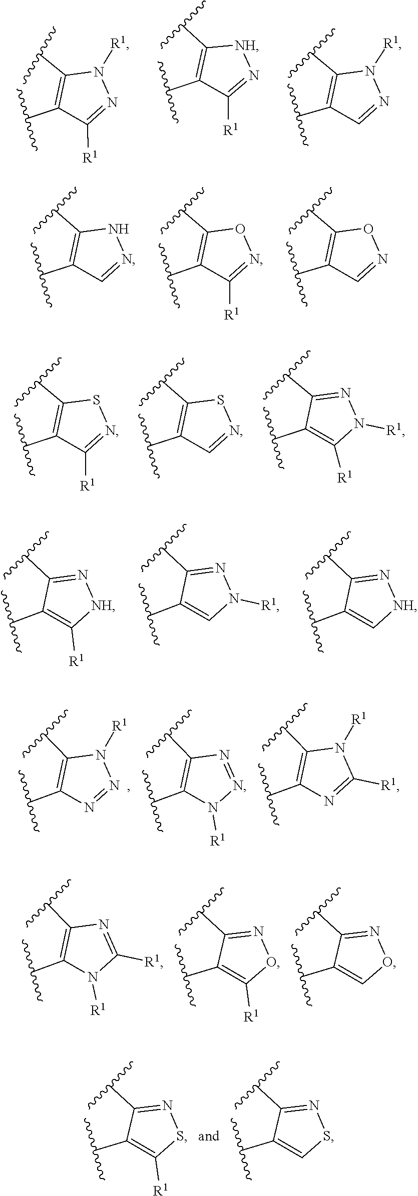
wherein each “ [symbol] ” represents a point of covalent attachment;
or a pharmaceutically acceptable salt, solvate, hydrate, or cocrystal thereof.

7. The compound of any one of the preceding claims, wherein ring A is selected from the group consisting of 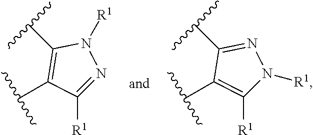
wherein each “ [symbol] ” represents a point of covalent attachment;
or a pharmaceutically acceptable salt, solvate, hydrate, or cocrystal thereof.

8. The compound of any one of the preceding claims, wherein ring A is selected from the group consisting of 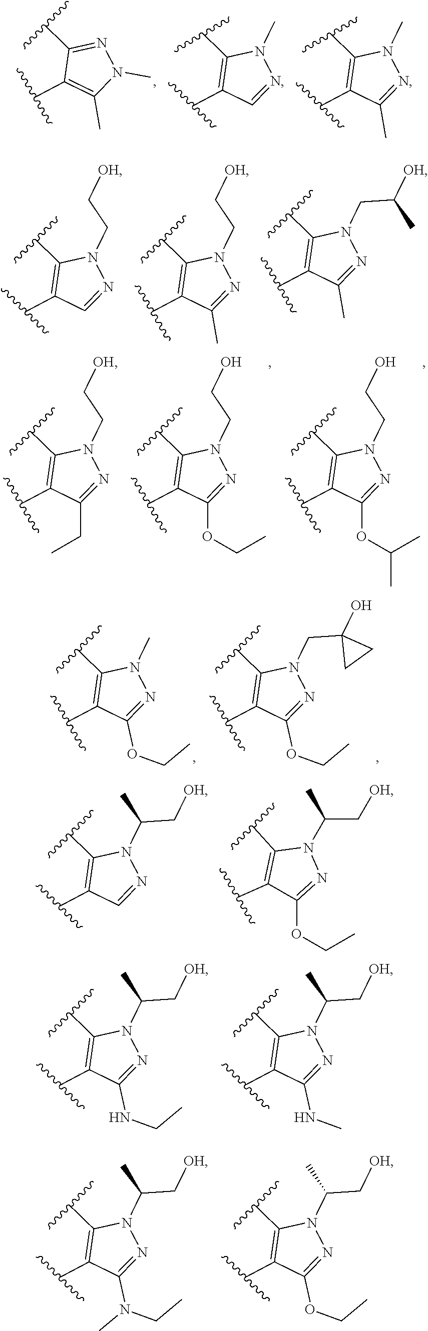 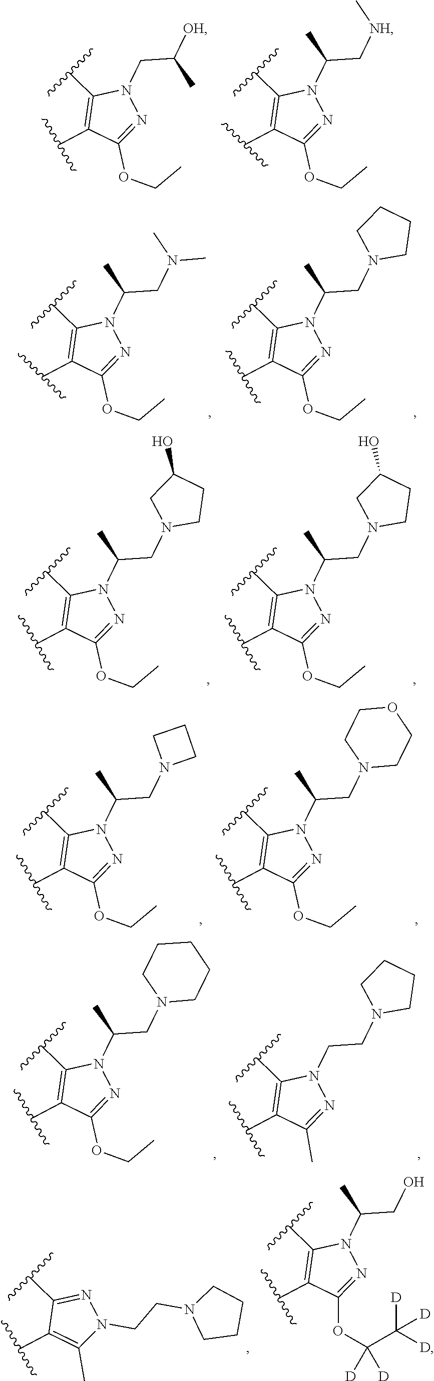 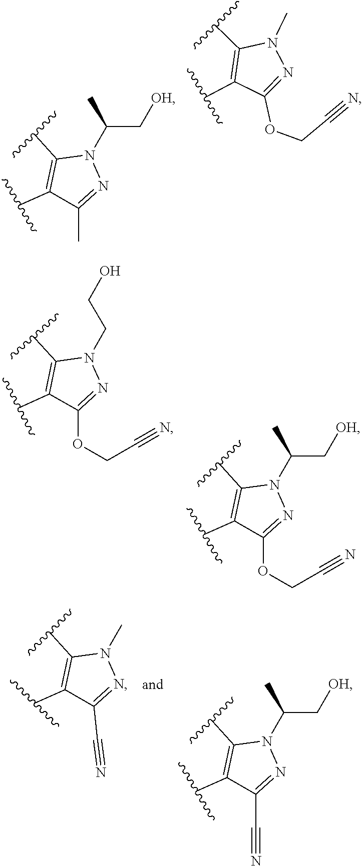
wherein each “ [symbol] ” represents a point of covalent attachment;
or a pharmaceutically acceptable salt, solvate, hydrate, or cocrystal thereof.

9. The compound of any one of claims 1 to 4 or 6, wherein ring B is selected from the group consisting of 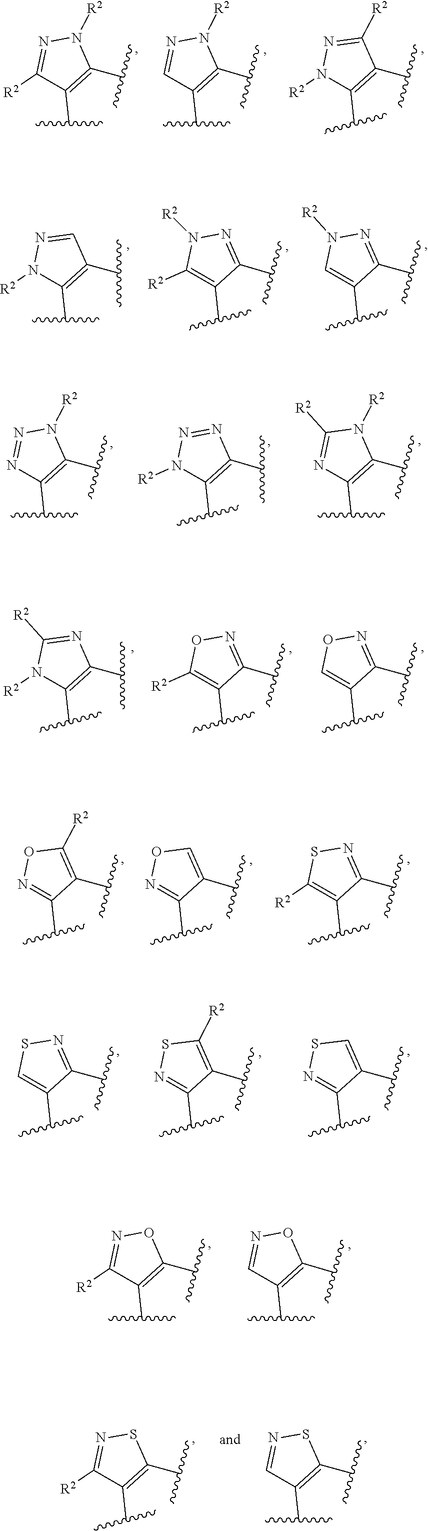
wherein each “ [symbol] ” represents a point of covalent attachment;
or a pharmaceutically acceptable salt, solvate, hydrate, or cocrystal thereof.

10. The compound of any one of the preceding claims, wherein ring B is selected from the group consisting of 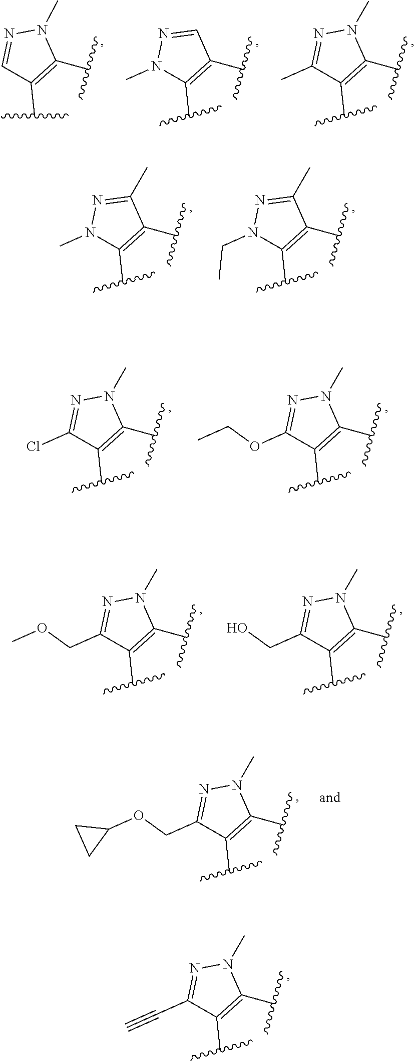
wherein each “ [symbol] ” represents a point of covalent attachment;
or a pharmaceutically acceptable salt, solvate, hydrate, or cocrystal thereof.

11. The compound of any one of the preceding claims, wherein p is 2 or 3, and the portion 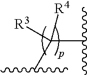
is an ethylene or propylene, wherein each R³ and R⁴ is independently H, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂; or at least one instance of R³ and R⁴, taken together with the carbon or carbons to which they are attached, combine to form C₃-C₆ cycloalkyl or 4- to 7-membered heterocycloalkyl, wherein each hydrogen atom in C₃-C₆ cycloalkyl and 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or R⁷ and one instance of R³ or R⁴, taken together with the atoms to which they are attached, combine to form 4- to 7-membered heterocycloalkyl, wherein each hydrogen atom in 4- to 7-membered heterocycloalkyl formed is independently optionally substituted by —OR^{d}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and each “ [symbol] ” represents a point of covalent attachment;
or a pharmaceutically acceptable salt, solvate, hydrate, or cocrystal thereof.

12. The compound of any one of the preceding claims, wherein one R³ is C₁-C₆ alkyl, wherein each hydrogen atom in C₁-C₆ alkyl is independently optionally substituted by deuterium, halogen, C₁-C₆ alkyl, C₁-C₆haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}—P(O)₂NR^{e}R^{f}, —P(O)OR^{e}, —P(O)₂OR^{e}, —CN, or —NO₂; and/or two R³ and R⁴, taken together with the carbon or carbons to which they are attached, combine to form C₃-C₆ cycloalkyl or 4- to 7-membered heterocycloalkyl, wherein each hydrogen atom in C₃-C₆ cycloalkyl and 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or R⁷ and one instance of R³ or R⁴ and, taken together with the atoms to which they are attached, combine to form a 4- to 7-membered heterocycloalkyl, wherein each hydrogen atom in 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and any remaining R³ and R⁴ are H or deuterium;
or a pharmaceutically acceptable salt, solvate, hydrate, or cocrystal thereof.

13. The compound of any one of the preceding claims, wherein one R³ is C₁-C₆ alkyl, wherein each hydrogen atom in C₁-C₆ alkyl is independently optionally substituted by deuterium, halogen, C₁-C₆alkyl, C₁-C₆haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and/or R⁷ and one instance of R³ or R⁴ taken together with the atoms to which they are attached, combine to form 4- to 7-membered heterocycloalkyl, wherein each hydrogen atom in 4- to 7-membered heterocycloalkyl is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; and
any remaining R³ and R⁴ are H or deuterium;
or a pharmaceutically acceptable salt, solvate, hydrate, or cocrystal thereof.

14. The compound of any one of the preceding claims, wherein one instance of R³ is C₁-C₆ alkyl; and any remaining R³ and R⁴ are H or deuterium;
or a pharmaceutically acceptable salt, solvate, hydrate, or cocrystal thereof.

15. The compound of any one of the preceding claims, wherein one instance of R³ is C₁-C₆ alkyl; R⁷ and one instance of R⁴, taken together with the atoms to which they are attached, combine to form a 4- to 7-membered heterocycloalkyl; and any remaining R³ and R⁴ are H or deuterium;
or a pharmaceutically acceptable salt, solvate, hydrate, or cocrystal thereof.

16. The compound of any one of the preceding claims, wherein one instance of R³ is methyl, and any remaining R³ and R⁴ are H or deuterium;
or a pharmaceutically acceptable salt, solvate, hydrate, or cocrystal thereof.

17. The compound of any one of the preceding claims, wherein R⁷ is H, C₁-C₆ alkyl, C₃-C₆ cycloalkyl, or —C(O)R^{c}; or R⁷ and one instance of R³ or R⁴, taken together with the atoms to which they are attached, combine to form 4- to 7-membered heterocycloalkyl; wherein each hydrogen atom in C₁-C₆ alkyl, C₃-C₆ cycloalkyl, and 4- to 7-membered heterocycloalkyl is independently optionally substituted by —OR^{c}, —OC(O)R^{c}, —OC(O)NR^{c}R^{d}, —OC(═NR^{d})NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═NR^{d})NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═NR^{d})NR^{c}R^{d}, —PR^{c}R^{d}, —P(O)R^{c}R^{d}, —P(O)₂R^{c}R^{d}, —P(O)NR^{c}R^{d}, —P(O)₂NR′R^{d}, —P(O)OR^{c}, —P(O)₂OR^{c}, —CN, or —NO₂;
or a pharmaceutically acceptable salt, solvate, hydrate, or cocrystal thereof.

18. The compound of any one of the preceding claims, wherein the portion 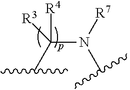
is of the formula 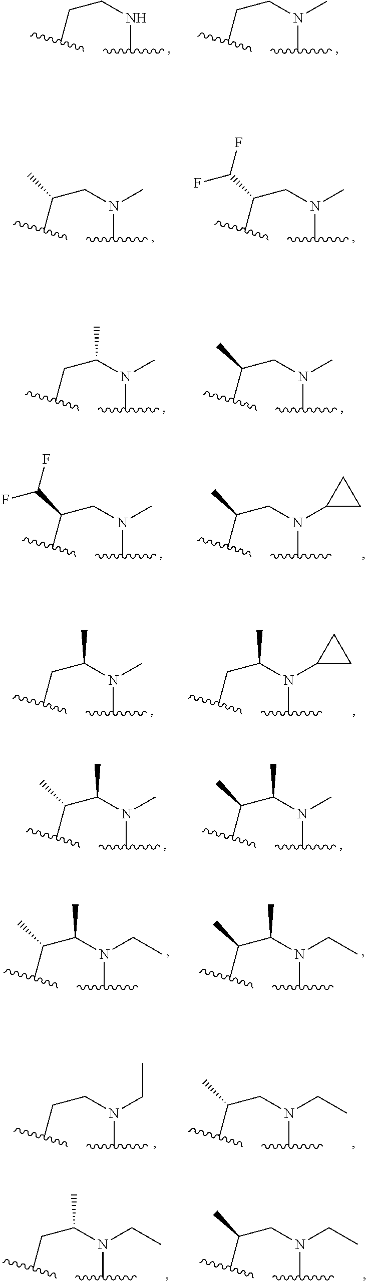 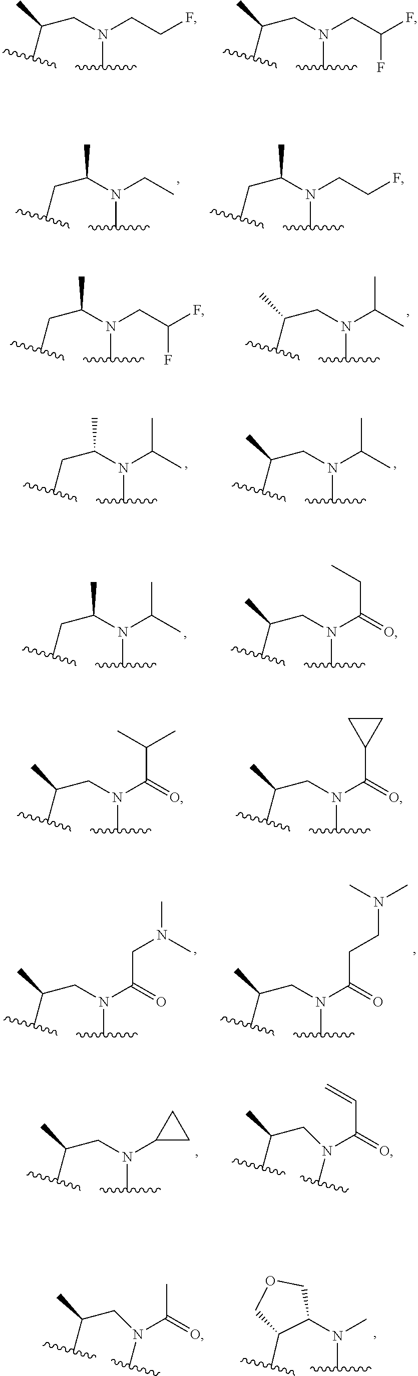 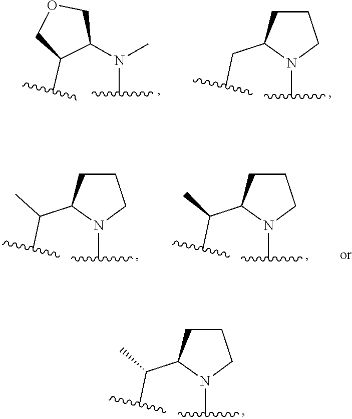
wherein each “ [symbol] ” represents a point of covalent attachment, and each hydrogen is independently optionally substituted with deuterium;
or a pharmaceutically acceptable salt, solvate, hydrate, or cocrystal thereof.

19. The compound of any one of the preceding claims, wherein R⁵ and R⁶ are each H;
or a pharmaceutically acceptable salt, solvate, hydrate, or cocrystal thereof.

20. The compound of any one of the preceding claims, wherein the portion 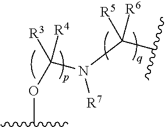
is of the formula 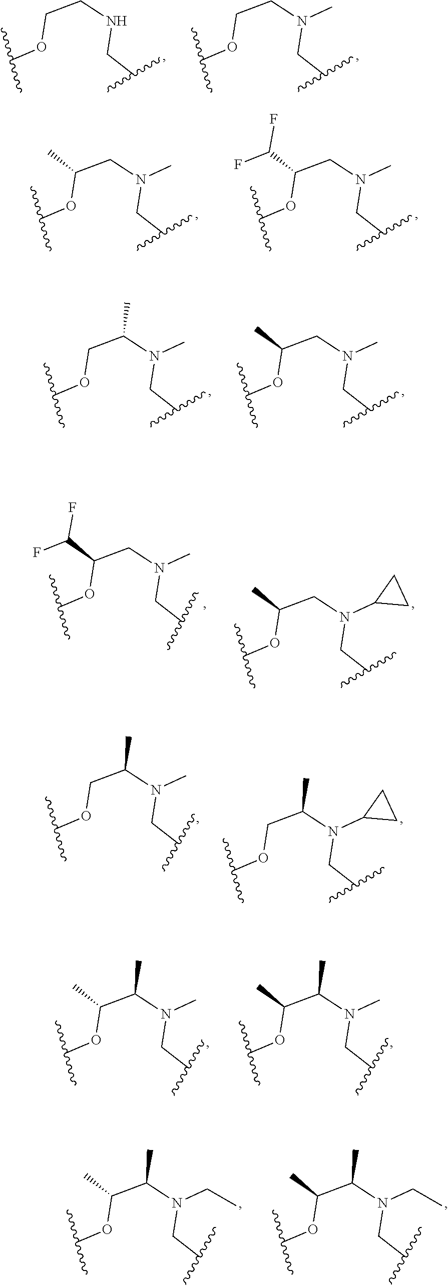 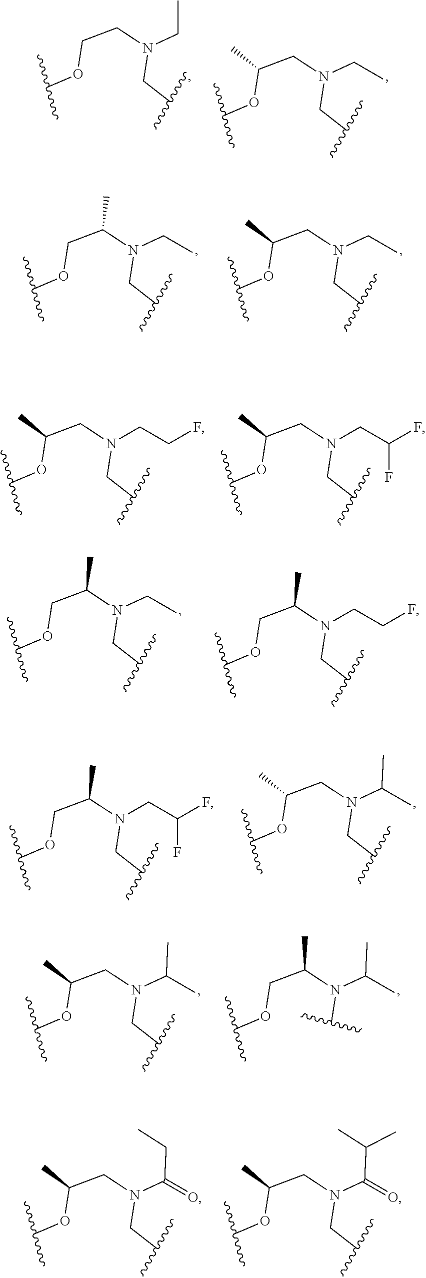 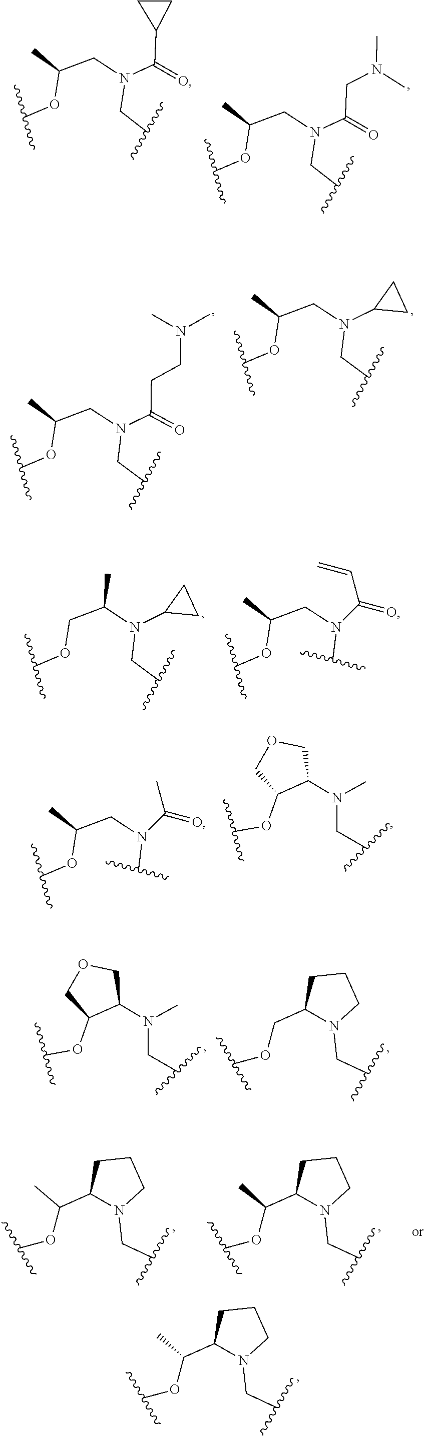
wherein each “ [symbol] ” represents a point of covalent attachment, and each hydrogen is independently optionally substituted with deuterium;
or a pharmaceutically acceptable salt, solvate, hydrate, or cocrystal thereof.

21. The compound of any one of the preceding claims, wherein R⁸ is H or C₁-C₆ alkyl;
or a pharmaceutically acceptable salt, solvate, hydrate, or cocrystal thereof.

22. The compound of any one of the preceding claims, wherein R⁹ is H, deuterium, halogen, C₁-C₆ alkyl, or —OR^{a}, wherein each hydrogen atom in C₁-C₆ alkyl is independently optionally substituted with deuterium;
or a pharmaceutically acceptable salt, solvate, hydrate, or cocrystal thereof.

23. The compound of any one of the preceding claims, wherein R⁹ is H, deuterium, halogen, —OCH₃, —CH₃, or —CD3;
or a pharmaceutically acceptable salt, solvate, hydrate, or cocrystal thereof.

24. The compound of claim 1, selected from the group consisting of 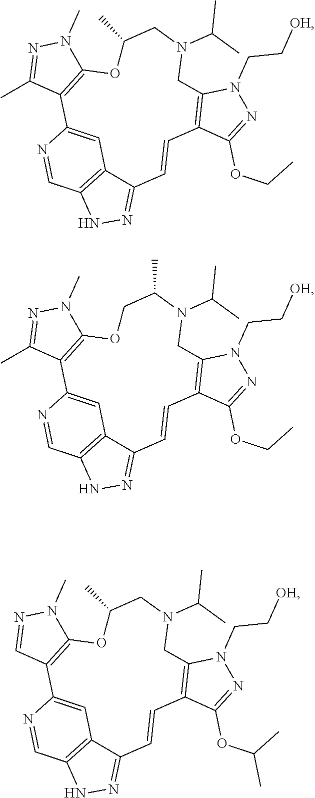 [SMILES: CCOc1nn(CCO)c2c1/C=C/c1n[nH]c3cnc(cc13)-c1c(C)nn(C)c1O[C@H](C)CN(C(C)C)C2 ; CCOc1nn(CCO)c2c1/C=C/c1n[nH]c3cnc(cc13)-c1c(C)nn(C)c1OC[C@H](C)N(C(C)C)C2 ; CC(C)Oc1nn(CCO)c2c1/C=C/c1n[nH]c3cnc(cc13)-c1cnn(C)c1O[C@H](C)CN(C(C)C)C2] 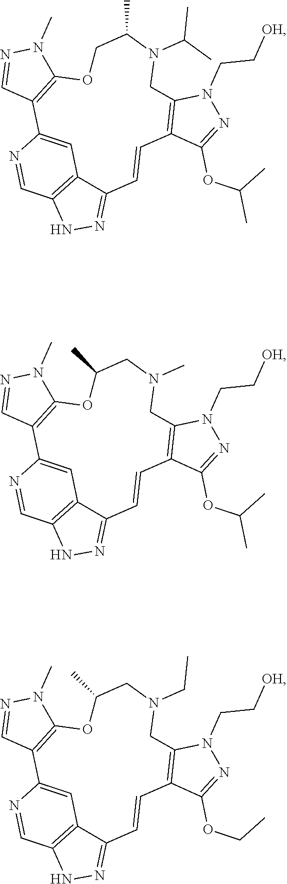 [SMILES: CC(C)Oc1nn(CCO)c2c1/C=C/c1n[nH]c3cnc(cc13)-c1cnn(C)c1OC[C@H](C)N(C(C)C)C2 ; CC(C)Oc1nn(CCO)c2c1/C=C/c1n[nH]c3cnc(cc13)-c1cnn(C)c1O[C@@H](C)CN(C)C2 ; CCOc1nn(CCO)c2c1/C=C/c1n[nH]c3cnc(cc13)-c1cnn(C)c1O[C@H](C)CN(CC)C2] 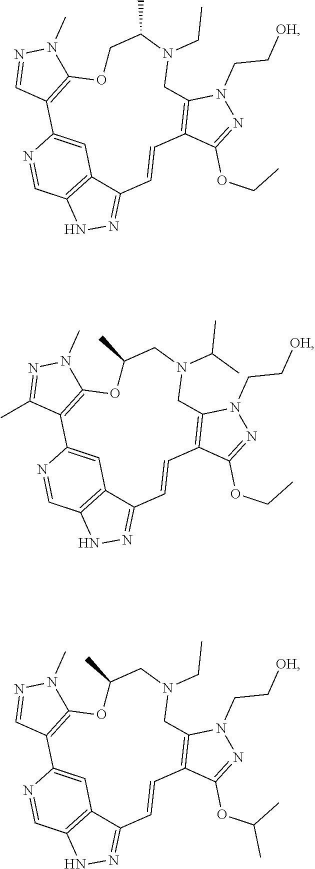 [SMILES: CCOc1nn(CCO)c2c1/C=C/c1n[nH]c3cnc(cc13)-c1cnn(C)c1OC[C@H](C)N(CC)C2 ; CCOc1nn(CCO)c2c1/C=C/c1n[nH]c3cnc(cc13)-c1c(C)nn(C)c1O[C@@H](C)CN(C(C)C)C2 ; CCN1Cc2c(c(OC(C)C)nn2CCO)/C=C/c2n[nH]c3cnc(cc23)-c2cnn(C)c2O[C@@H](C)C1] 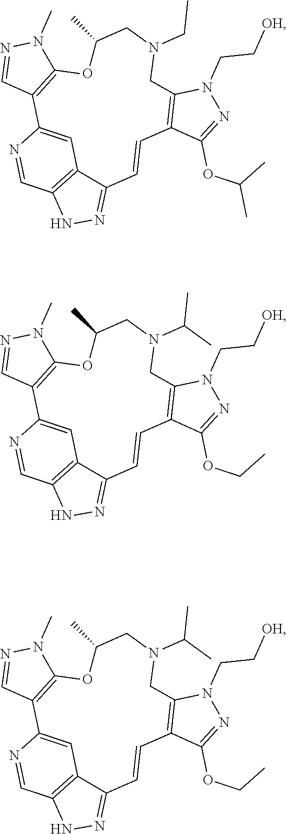 [SMILES: CCN1Cc2c(c(OC(C)C)nn2CCO)/C=C/c2n[nH]c3cnc(cc23)-c2cnn(C)c2O[C@H](C)C1 ; CCOc1nn(CCO)c2c1/C=C/c1n[nH]c3cnc(cc13)-c1cnn(C)c1O[C@@H](C)CN(C(C)C)C2 ; CCOc1nn(CCO)c2c1/C=C/c1n[nH]c3cnc(cc13)-c1cnn(C)c1O[C@H](C)CN(C(C)C)C2] 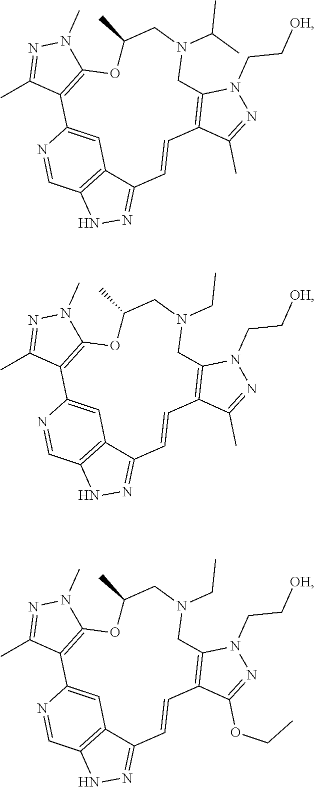 [SMILES: Cc1nn(CCO)c2c1/C=C/c1n[nH]c3cnc(cc13)-c1c(C)nn(C)c1O[C@@H](C)CN(C(C)C)C2 ; CCN1Cc2c(c(C)nn2CCO)/C=C/c2n[nH]c3cnc(cc23)-c2c(C)nn(C)c2O[C@H](C)C1 ; CCOc1nn(CCO)c2c1/C=C/c1n[nH]c3cnc(cc13)-c1c(C)nn(C)c1O[C@@H](C)CN(CC)C2] 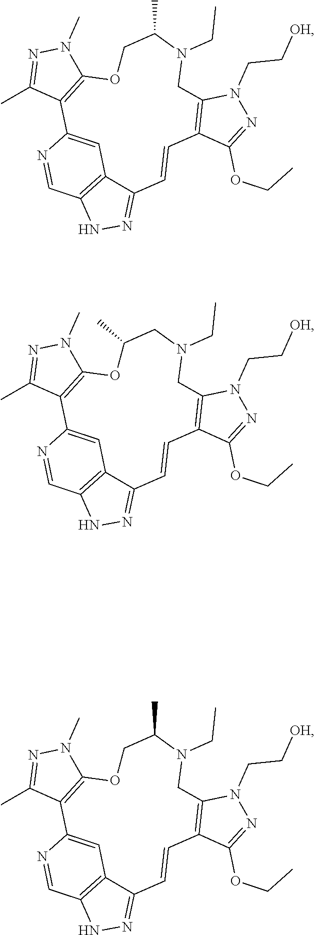 [SMILES: CCOc1nn(CCO)c2c1/C=C/c1n[nH]c3cnc(cc13)-c1c(C)nn(C)c1OC[C@H](C)N(CC)C2 ; CCOc1nn(CCO)c2c1/C=C/c1n[nH]c3cnc(cc13)-c1c(C)nn(C)c1O[C@H](C)CN(CC)C2 ; CCOc1nn(CCO)c2c1/C=C/c1n[nH]c3cnc(cc13)-c1c(C)nn(C)c1OC[C@@H](C)N(CC)C2] 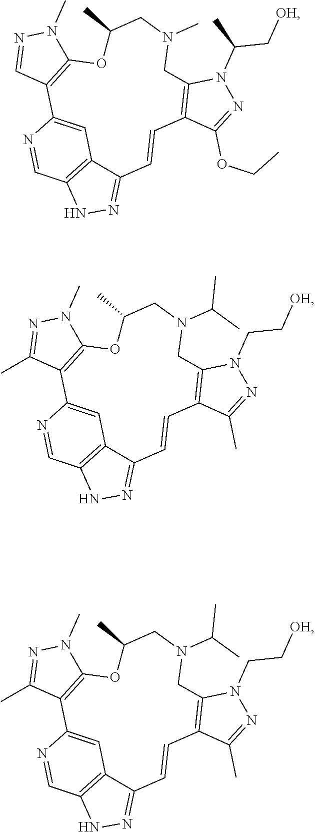 [SMILES: CCOc1nn([C@@H](C)CO)c2c1/C=C/c1n[nH]c3cnc(cc13)-c1cnn(C)c1O[C@@H](C)CN(C)C2 ; Cc1nn(CCO)c2c1/C=C/c1n[nH]c3cnc(cc13)-c1c(C)nn(C)c1O[C@H](C)CN(C(C)C)C2 ; Cc1nn(CCO)c2c1/C=C/c1n[nH]c3cnc(cc13)-c1c(C)nn(C)c1O[C@@H](C)CN(C(C)C)C2] 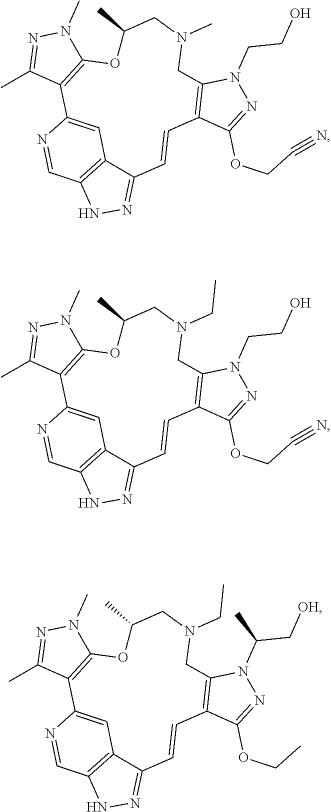 [SMILES: Cc1nn(C)c2c1-c1cc3c(n[nH]c3cn1)/C=C/c1c(OCC#N)nn(CCO)c1CN(C)C[C@H](C)O2 ; CCN1Cc2c(c(OCC#N)nn2CCO)/C=C/c2n[nH]c3cnc(cc23)-c2c(C)nn(C)c2O[C@@H](C)C1 ; CCOc1nn([C@@H](C)CO)c2c1/C=C/c1n[nH]c3cnc(cc13)-c1c(C)nn(C)c1O[C@H](C)CN(CC)C2] 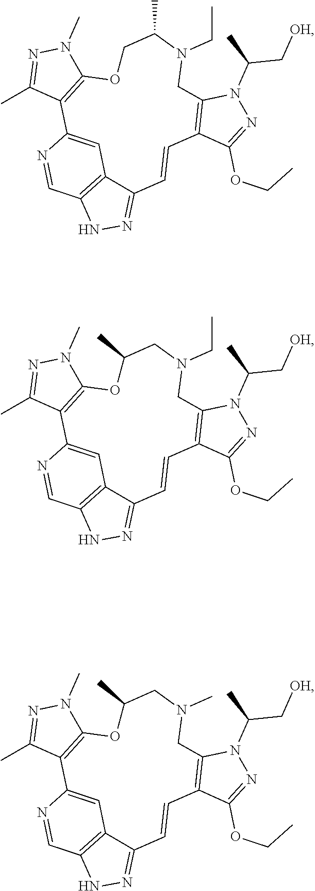 [SMILES: CCOc1nn([C@@H](C)CO)c2c1/C=C/c1n[nH]c3cnc(cc13)-c1c(C)nn(C)c1OC[C@H](C)N(CC)C2 ; CCOc1nn([C@@H](C)CO)c2c1/C=C/c1n[nH]c3cnc(cc13)-c1c(C)nn(C)c1O[C@@H](C)CN(CC)C2 ; CCOc1nn([C@@H](C)CO)c2c1/C=C/c1n[nH]c3cnc(cc13)-c1c(C)nn(C)c1O[C@@H](C)CN(C)C2] 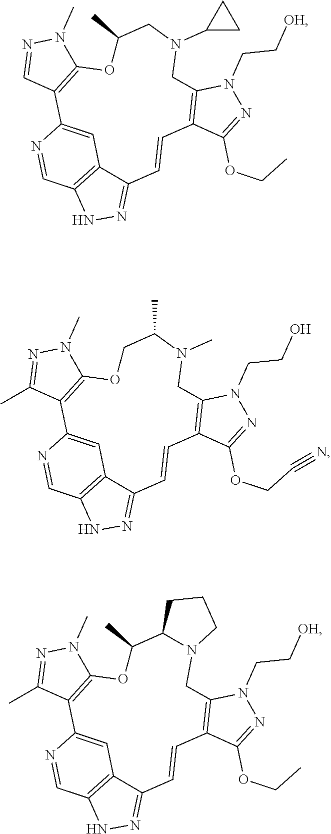 [SMILES: CCOc1nn(CCO)c2c1/C=C/c1n[nH]c3cnc(cc13)-c1cnn(C)c1O[C@@H](C)CN(C1CC1)C2 ; Cc1nn(C)c2c1-c1cc3c(n[nH]c3cn1)/C=C/c1c(OCC#N)nn(CCO)c1CN(C)[C@@H](C)CO2 ; CCOc1nn(CCO)c2c1/C=C/c1n[nH]c3cnc(cc13)-c1c(C)nn(C)c1O[C@@H](C)[C@H]1CCCN1C2] 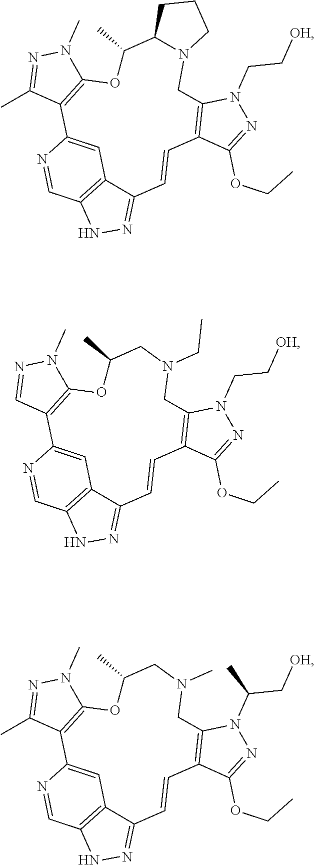 [SMILES: CCOc1nn(CCO)c2c1/C=C/c1n[nH]c3cnc(cc13)-c1c(C)nn(C)c1O[C@H](C)[C@H]1CCCN1C2 ; CCOc1nn(CCO)c2c1/C=C/c1n[nH]c3cnc(cc13)-c1cnn(C)c1O[C@@H](C)CN(CC)C2 ; CCOc1nn([C@@H](C)CO)c2c1/C=C/c1n[nH]c3cnc(cc13)-c1c(C)nn(C)c1O[C@H](C)CN(C)C2] 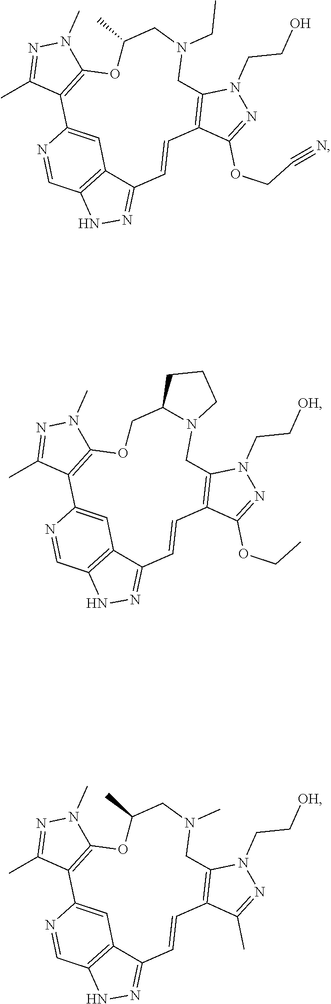 [SMILES: CCN1Cc2c(c(OCC#N)nn2CCO)/C=C/c2n[nH]c3cnc(cc23)-c2c(C)nn(C)c2O[C@H](C)C1 ; CCOc1nn(CCO)c2c1/C=C/c1n[nH]c3cnc(cc13)-c1c(C)nn(C)c1OC[C@H]1CCCN1C2 ; Cc1nn(CCO)c2c1/C=C/c1n[nH]c3cnc(cc13)-c1c(C)nn(C)c1O[C@@H](C)CN(C)C2] 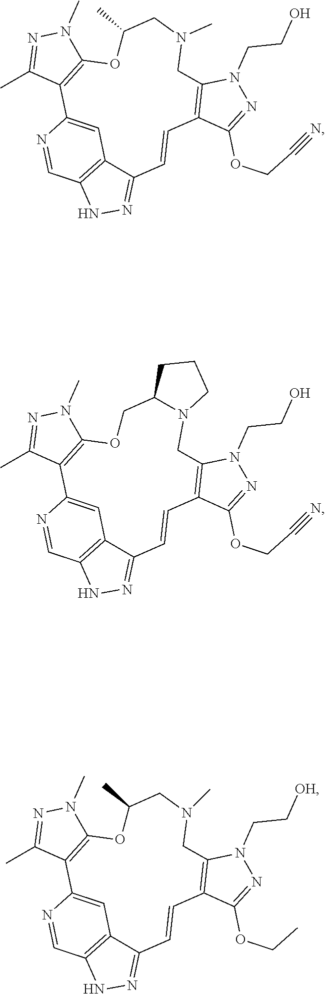 [SMILES: Cc1nn(C)c2c1-c1cc3c(n[nH]c3cn1)/C=C/c1c(OCC#N)nn(CCO)c1CN(C)C[C@@H](C)O2 ; Cc1nn(C)c2c1-c1cc3c(n[nH]c3cn1)/C=C/c1c(OCC#N)nn(CCO)c1CN1CCC[C@@H]1CO2 ; CCOc1nn(CCO)c2c1/C=C/c1n[nH]c3cnc(cc13)-c1c(C)nn(C)c1O[C@@H](C)CN(C)C2] 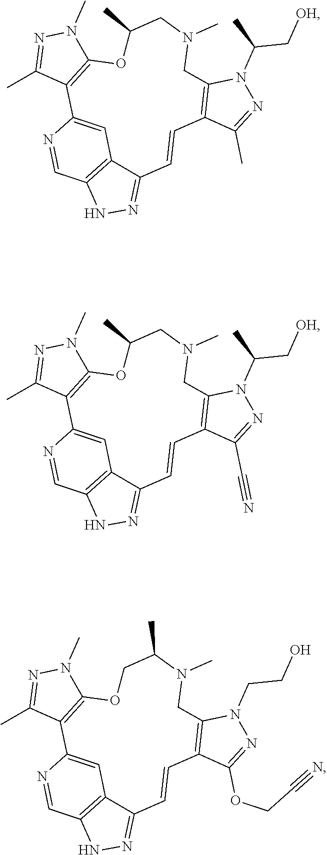 [SMILES: Cc1nn([C@@H](C)CO)c2c1/C=C/c1n[nH]c3cnc(cc13)-c1c(C)nn(C)c1O[C@@H](C)CN(C)C2 ; Cc1nn(C)c2c1-c1cc3c(n[nH]c3cn1)/C=C/c1c(C#N)nn([C@@H](C)CO)c1CN(C)C[C@H](C)O2 ; Cc1nn(C)c2c1-c1cc3c(n[nH]c3cn1)/C=C/c1c(OCC#N)nn(CCO)c1CN(C)[C@H](C)CO2] 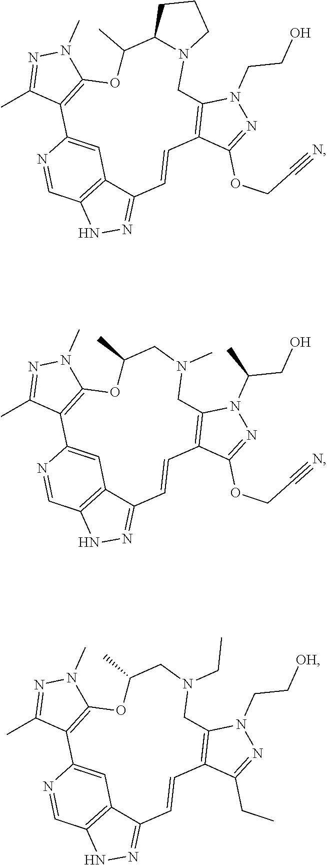 [SMILES: Cc1nn(C)c2c1-c1cc3c(n[nH]c3cn1)/C=C/c1c(OCC#N)nn(CCO)c1CN1CCC[C@@H]1C(C)O2 ; Cc1nn(C)c2c1-c1cc3c(n[nH]c3cn1)/C=C/c1c(OCC#N)nn([C@@H](C)CO)c1CN(C)C[C@H](C)O2 ; CCc1nn(CCO)c2c1/C=C/c1n[nH]c3cnc(cc13)-c1c(C)nn(C)c1O[C@H](C)CN(CC)C2] 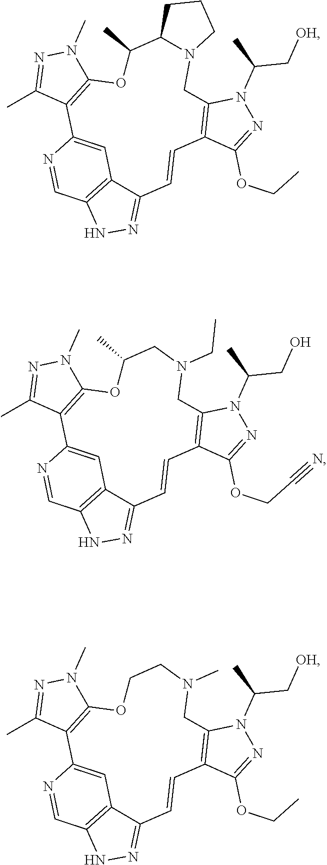 [SMILES: CCOc1nn([C@@H](C)CO)c2c1/C=C/c1n[nH]c3cnc(cc13)-c1c(C)nn(C)c1O[C@@H](C)[C@H]1CCCN1C2 ; CCN1Cc2c(c(OCC#N)nn2[C@@H](C)CO)/C=C/c2n[nH]c3cnc(cc23)-c2c(C)nn(C)c2O[C@H](C)C1 ; CCOc1nn([C@@H](C)CO)c2c1/C=C/c1n[nH]c3cnc(cc13)-c1c(C)nn(C)c1OCCN(C)C2] 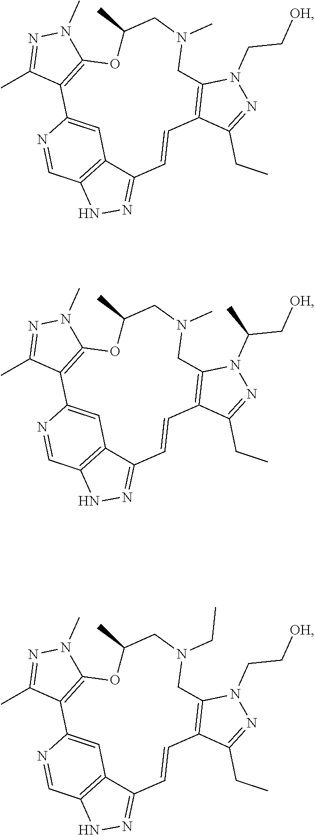 [SMILES: CCc1nn(CCO)c2c1/C=C/c1n[nH]c3cnc(cc13)-c1c(C)nn(C)c1O[C@@H](C)CN(C)C2 ; CCc1nn([C@@H](C)CO)c2c1/C=C/c1n[nH]c3cnc(cc13)-c1c(C)nn(C)c1O[C@@H](C)CN(C)C2 ; CCc1nn(CCO)c2c1/C=C/c1n[nH]c3cnc(cc13)-c1c(C)nn(C)c1O[C@@H](C)CN(CC)C2] 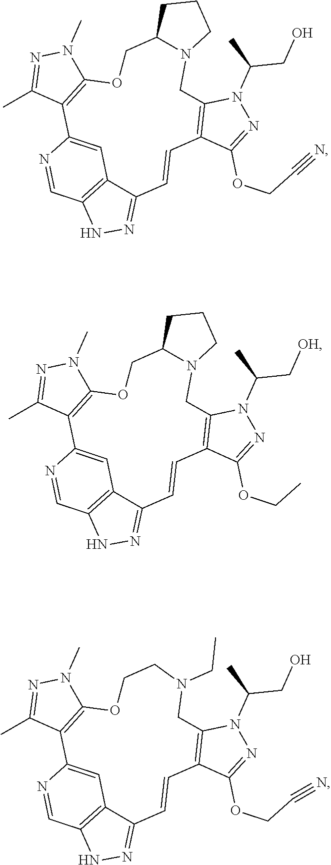 [SMILES: Cc1nn(C)c2c1-c1cc3c(n[nH]c3cn1)/C=C/c1c(OCC#N)nn([C@@H](C)CO)c1CN1CCC[C@@H]1CO2 ; CCOc1nn([C@@H](C)CO)c2c1/C=C/c1n[nH]c3cnc(cc13)-c1c(C)nn(C)c1OC[C@H]1CCCN1C2 ; CCN1CCOc2c(c(C)nn2C)-c2cc3c(n[nH]c3cn2)/C=C/c2c(OCC#N)nn([C@@H](C)CO)c2C1] 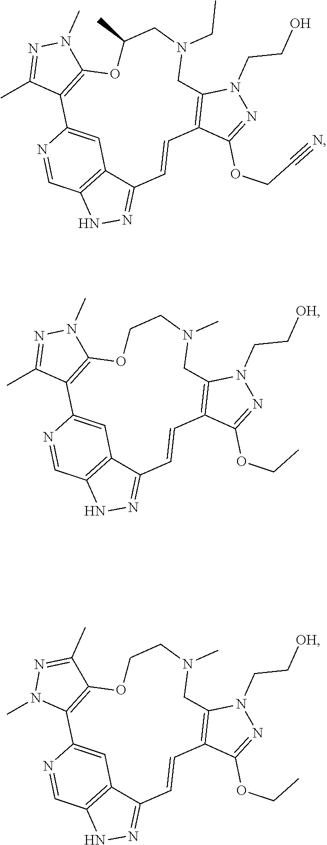 [SMILES: CCN1Cc2c(c(OCC#N)nn2CCO)/C=C/c2n[nH]c3cnc(cc23)-c2c(C)nn(C)c2O[C@@H](C)C1 ; CCOc1nn(CCO)c2c1/C=C/c1n[nH]c3cnc(cc13)-c1c(C)nn(C)c1OCCN(C)C2 ; CCOc1nn(CCO)c2c1/C=C/c1n[nH]c3cnc(cc13)-c1c(c(C)nn1C)OCCN(C)C2] 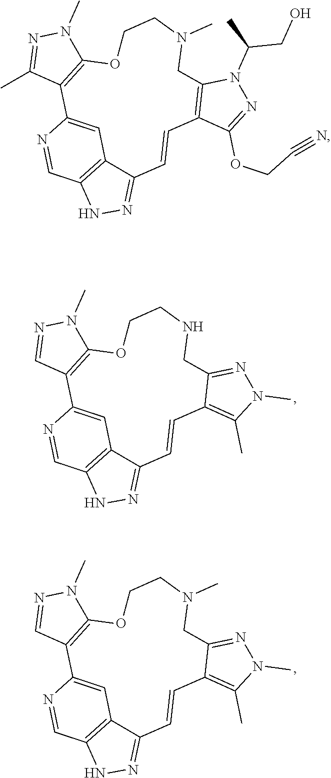 [SMILES: Cc1nn(C)c2c1-c1cc3c(n[nH]c3cn1)/C=C/c1c(OCC#N)nn([C@@H](C)CO)c1CN(C)CCO2 ; Cc1c2c(nn1C)CNCCOc1c(cnn1C)-c1cc3c(n[nH]c3cn1)/C=C/2 ; Cc1c2c(nn1C)CN(C)CCOc1c(cnn1C)-c1cc3c(n[nH]c3cn1)/C=C/2] 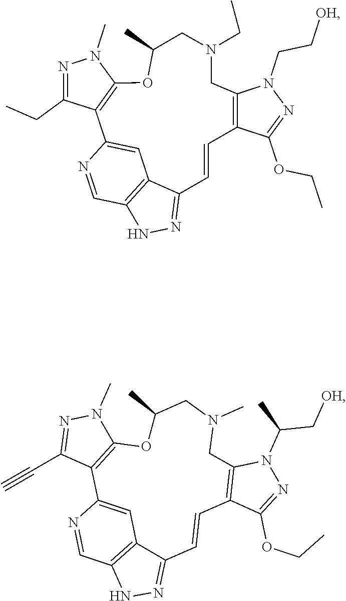 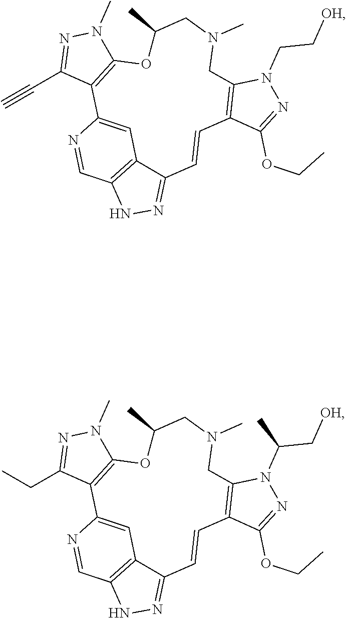 [SMILES: C#Cc1nn(C)c2c1-c1cc3c(n[nH]c3cn1)/C=C/c1c(OCC)nn(CCO)c1CN(C)C[C@H](C)O2 ; CCOc1nn([C@@H](C)CO)c2c1/C=C/c1n[nH]c3cnc(cc13)-c1c(CC)nn(C)c1O[C@@H](C)CN(C)C2] 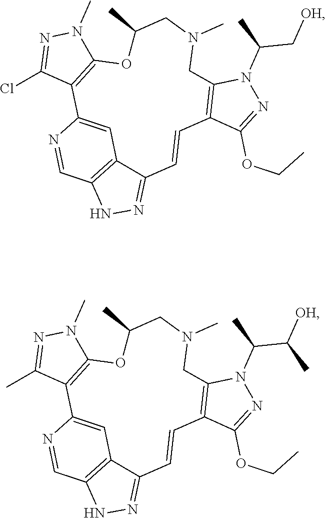 [SMILES: CCOc1nn([C@@H](C)CO)c2c1/C=C/c1n[nH]c3cnc(cc13)-c1c(Cl)nn(C)c1O[C@@H](C)CN(C)C2 ; CCOc1nn([C@@H](C)[C@H](C)O)c2c1/C=C/c1n[nH]c3cnc(cc13)-c1c(C)nn(C)c1O[C@@H](C)CN(C)C2] 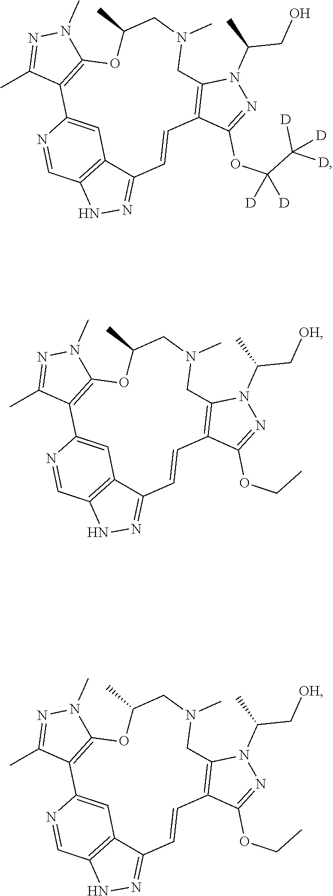 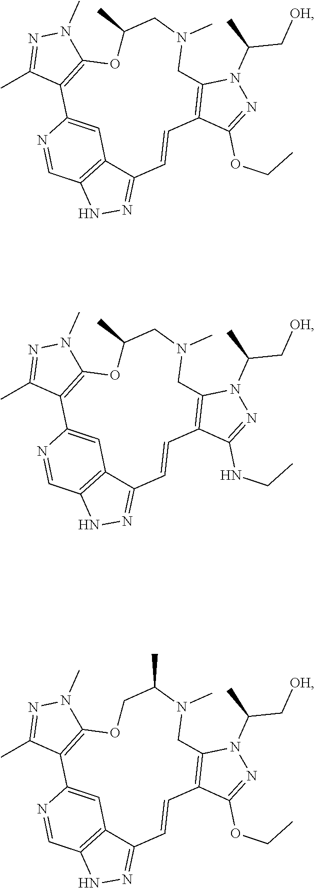 [SMILES: CCOc1nn([C@@H](C)CO)c2c1/C=C/c1n[nH]c3cnc(cc13)-c1c(C)nn(C)c1O[C@@H](C)CN(C)C2 ; CCNc1nn([C@@H](C)CO)c2c1/C=C/c1n[nH]c3cnc(cc13)-c1c(C)nn(C)c1O[C@@H](C)CN(C)C2 ; CCOc1nn([C@@H](C)CO)c2c1/C=C/c1n[nH]c3cnc(cc13)-c1c(C)nn(C)c1OC[C@@H](C)N(C)C2] 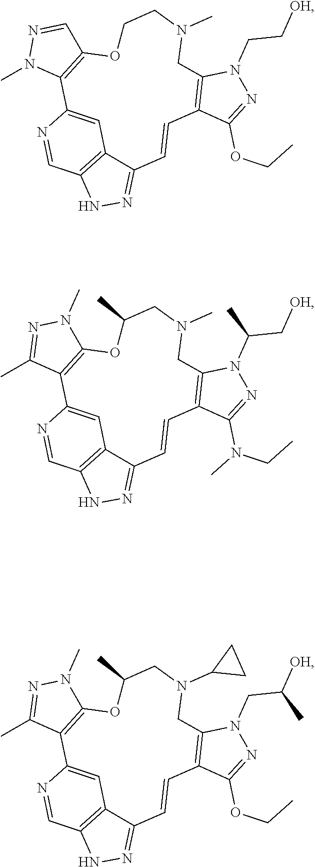 [SMILES: CCOc1nn(CCO)c2c1/C=C/c1n[nH]c3cnc(cc13)-c1c(cnn1C)OCCN(C)C2 ; CCN(C)c1nn([C@@H](C)CO)c2c1/C=C/c1n[nH]c3cnc(cc13)-c1c(C)nn(C)c1O[C@@H](C)CN(C)C2 ; CCOc1nn(C[C@H](C)O)c2c1/C=C/c1n[nH]c3cnc(cc13)-c1c(C)nn(C)c1O[C@@H](C)CN(C1CC1)C2] 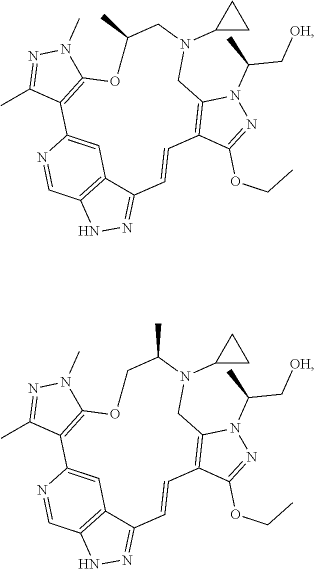 [SMILES: CCOc1nn([C@@H](C)CO)c2c1/C=C/c1n[nH]c3cnc(cc13)-c1c(C)nn(C)c1O[C@@H](C)CN(C1CC1)C2 ; CCOc1nn([C@@H](C)CO)c2c1/C=C/c1n[nH]c3cnc(cc13)-c1c(C)nn(C)c1OC[C@@H](C)N(C1CC1)C2] 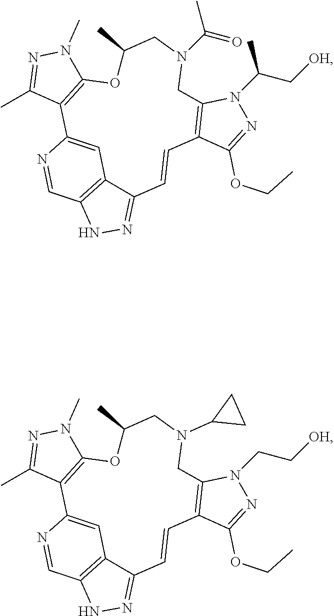 [SMILES: CCOc1nn([C@@H](C)CO)c2c1/C=C/c1n[nH]c3cnc(cc13)-c1c(C)nn(C)c1O[C@@H](C)CN(C(C)=O)C2 ; CCOc1nn(CCO)c2c1/C=C/c1n[nH]c3cnc(cc13)-c1c(C)nn(C)c1O[C@@H](C)CN(C1CC1)C2] 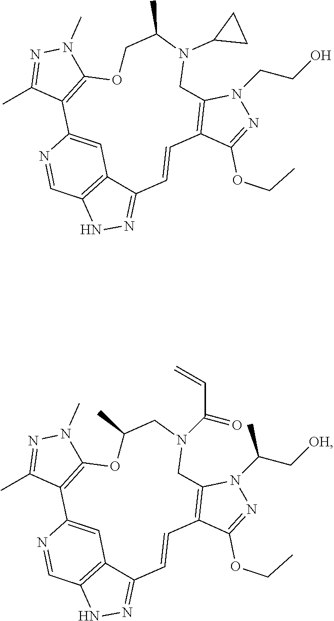 [SMILES: CCOc1nn(CCO)c2c1/C=C/c1n[nH]c3cnc(cc13)-c1c(C)nn(C)c1OC[C@@H](C)N(C1CC1)C2 ; C=CC(=O)N1Cc2c(c(OCC)nn2[C@@H](C)CO)/C=C/c2n[nH]c3cnc(cc23)-c2c(C)nn(C)c2O[C@@H](C)C1] 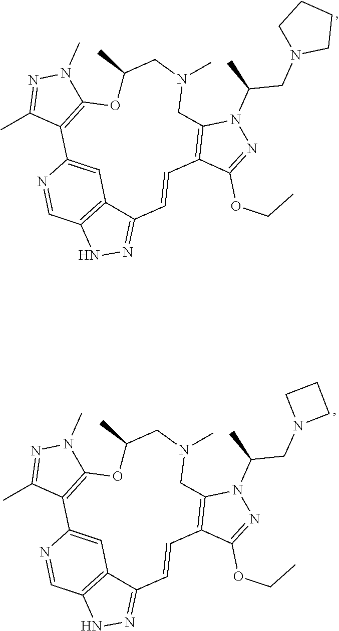 [SMILES: CCOc1nn([C@@H](C)CN2CCCC2)c2c1/C=C/c1n[nH]c3cnc(cc13)-c1c(C)nn(C)c1O[C@@H](C)CN(C)C2 ; CCOc1nn([C@@H](C)CN2CCC2)c2c1/C=C/c1n[nH]c3cnc(cc13)-c1c(C)nn(C)c1O[C@@H](C)CN(C)C2] 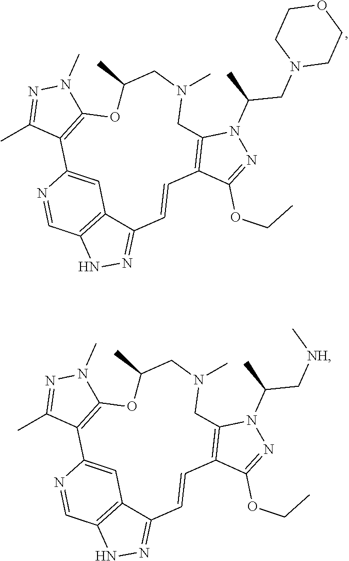 [SMILES: CCOc1nn([C@@H](C)CN2CCOCC2)c2c1/C=C/c1n[nH]c3cnc(cc13)-c1c(C)nn(C)c1O[C@@H](C)CN(C)C2 ; CCOc1nn([C@@H](C)CNC)c2c1/C=C/c1n[nH]c3cnc(cc13)-c1c(C)nn(C)c1O[C@@H](C)CN(C)C2] 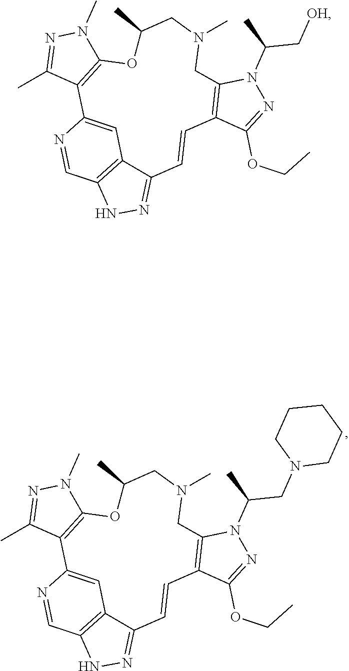 [SMILES: CCOc1nn([C@@H](C)CO)c2c1/C=C/c1n[nH]c3cnc(cc13)-c1c(C)nn(C)c1O[C@@H](C)CN(C)C2 ; CCOc1nn([C@@H](C)CN2CCCCC2)c2c1/C=C/c1n[nH]c3cnc(cc13)-c1c(C)nn(C)c1O[C@@H](C)CN(C)C2] 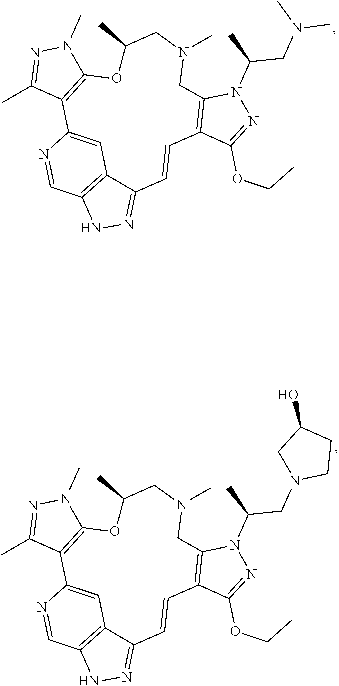 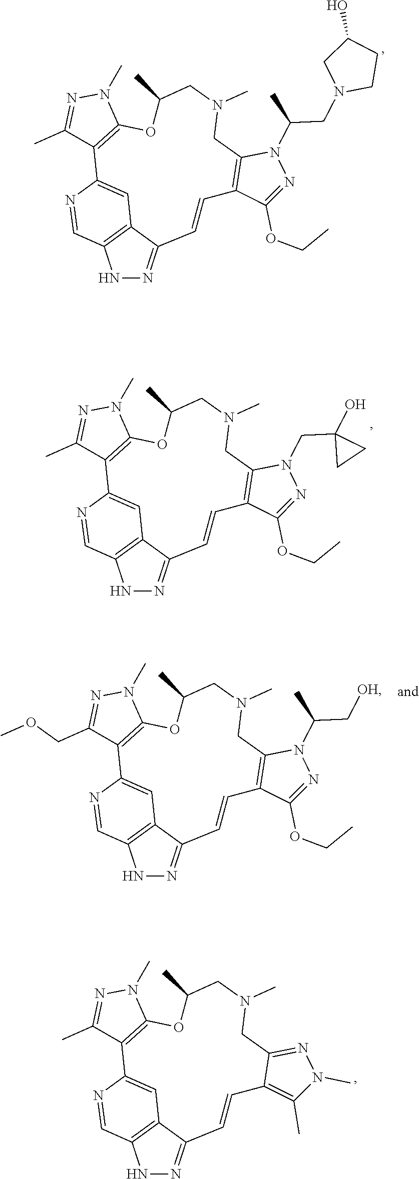
or a pharmaceutically acceptable salt, solvate, hydrate, or cocrystal thereof.

25. The compound of claim 1, selected from the group consisting of 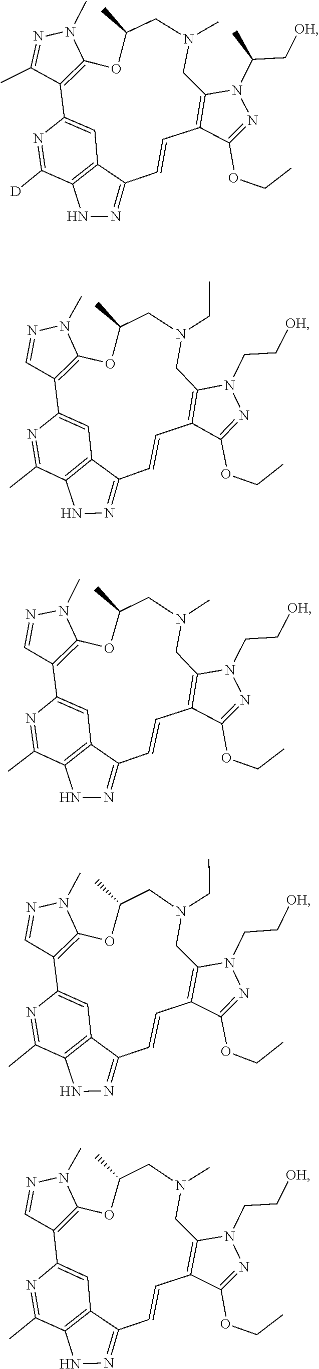 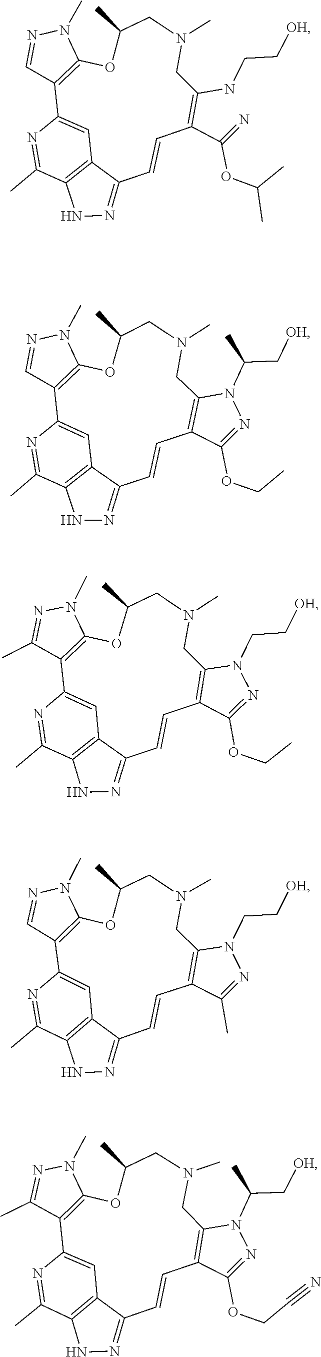 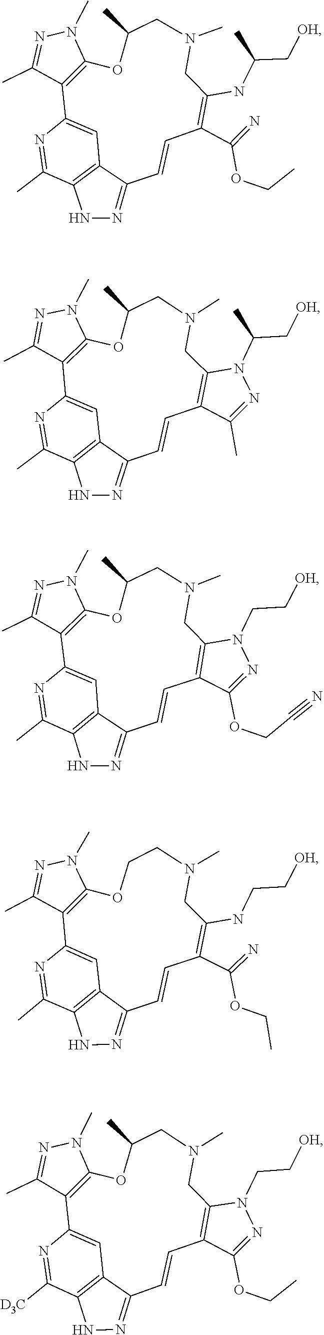 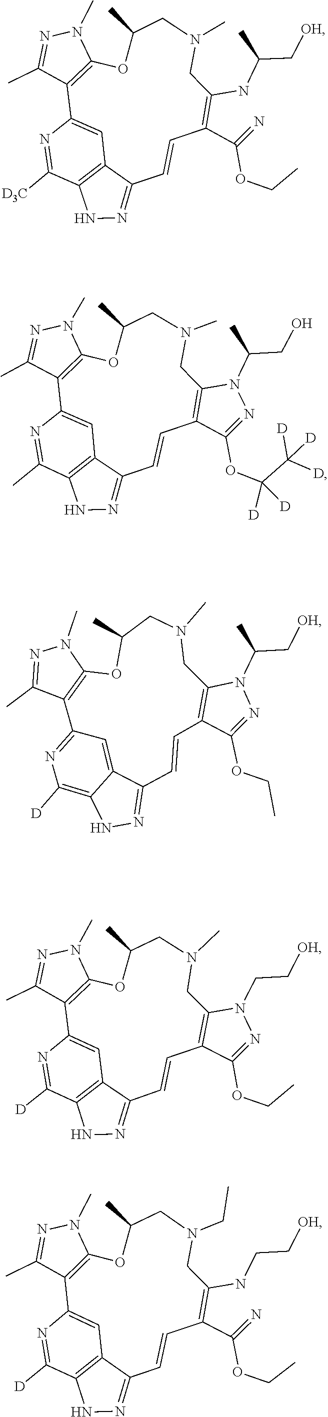 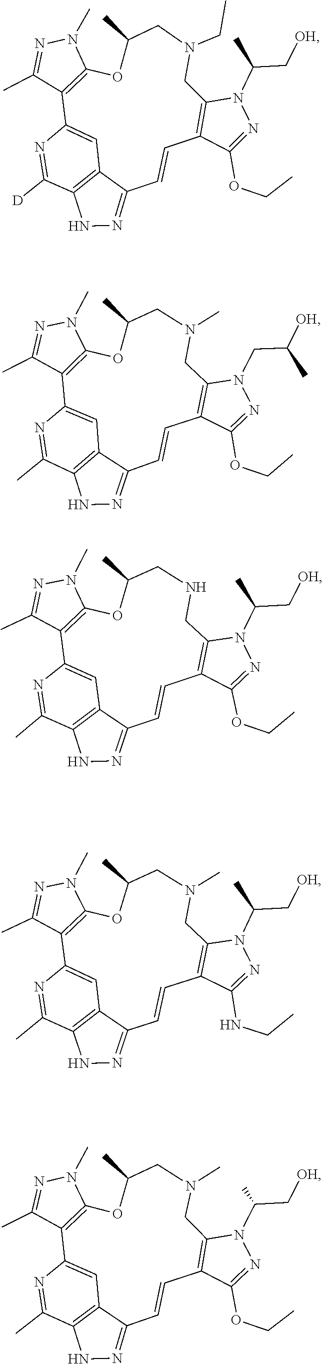 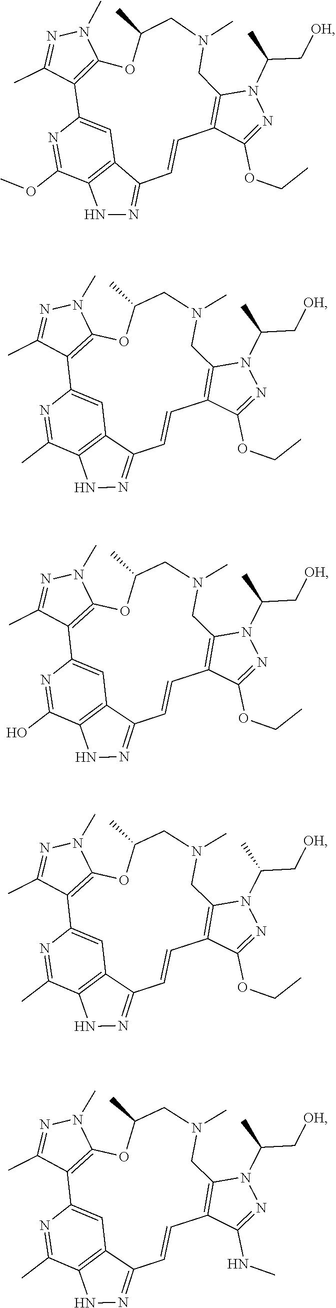 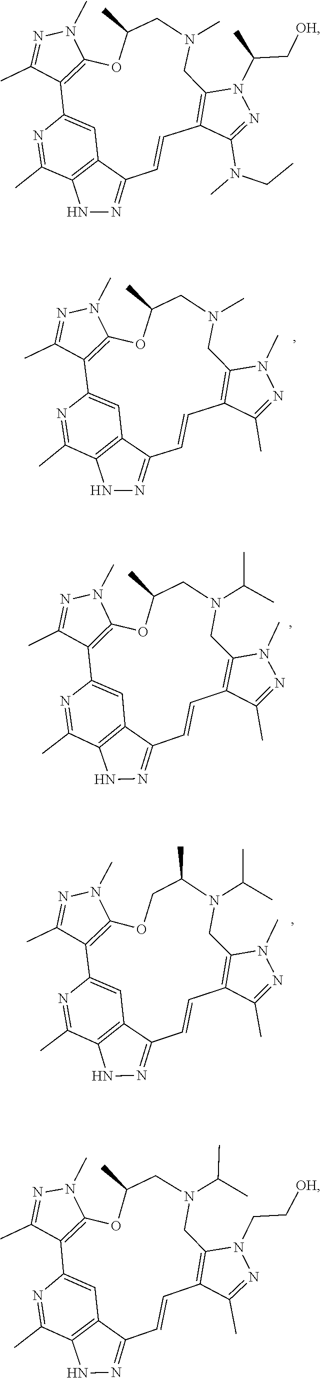 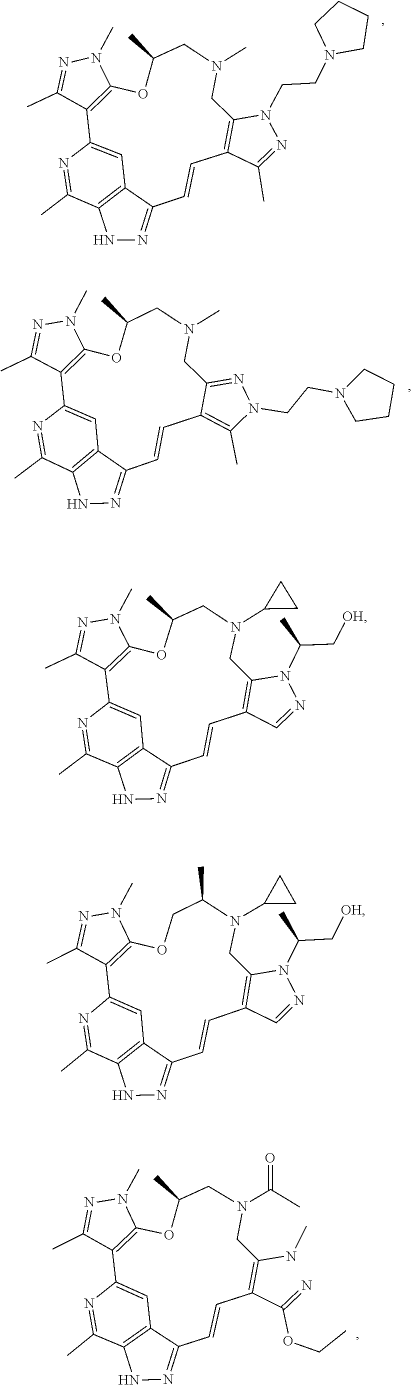 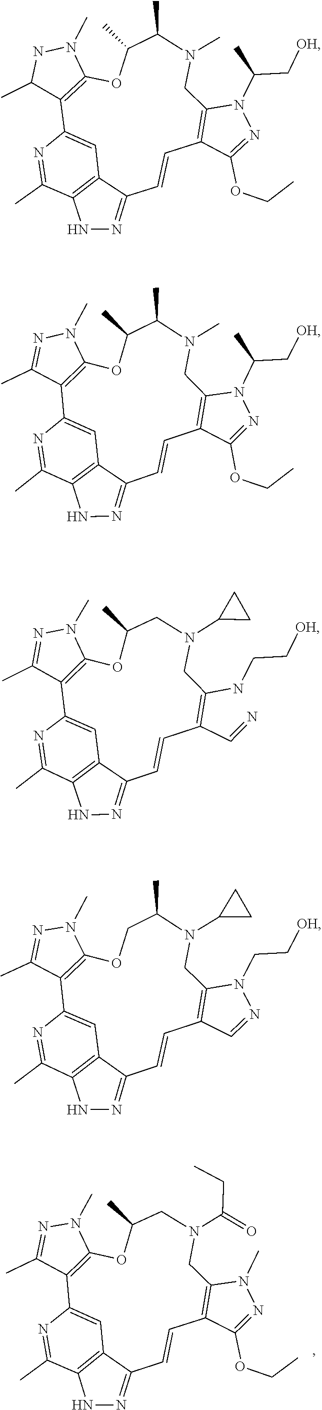 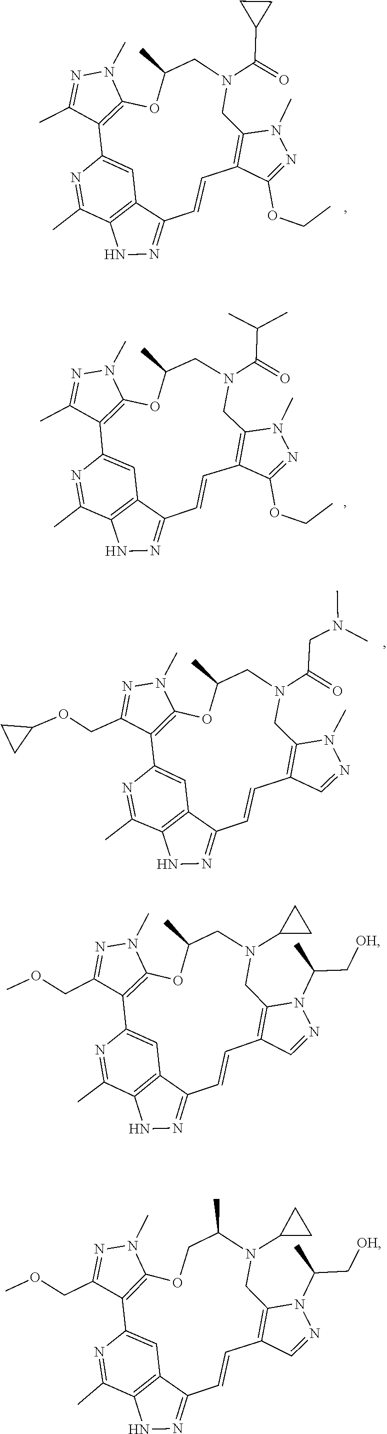 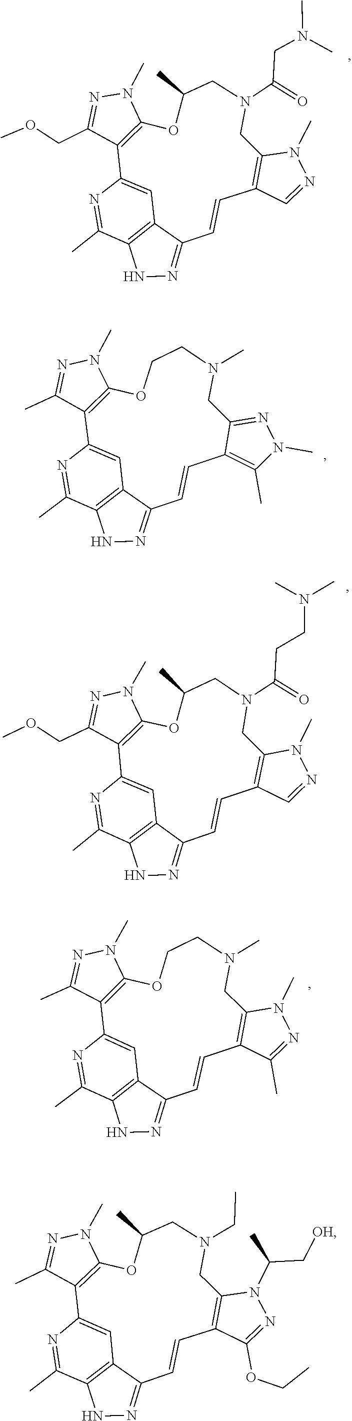 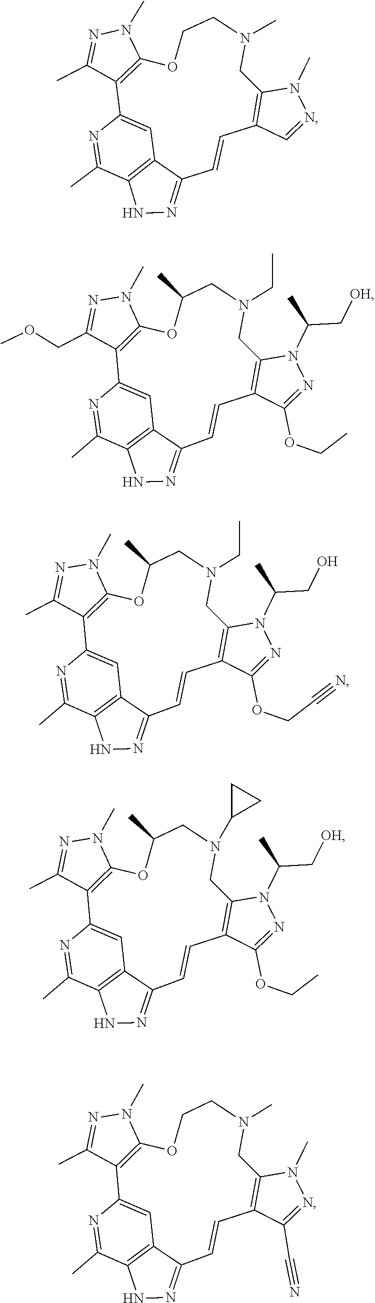 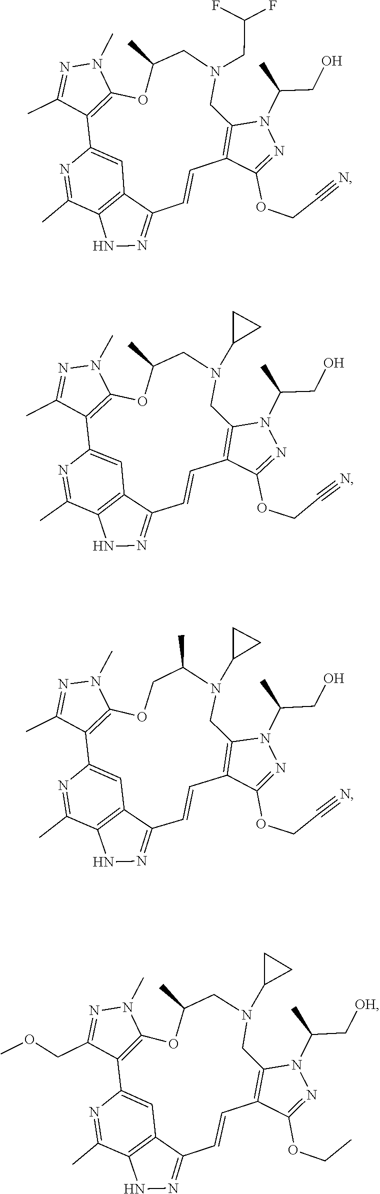 [SMILES: Cc1nn(C)c2c1-c1cc3c(n[nH]c3c(C)n1)/C=C/c1c(OCC#N)nn([C@@H](C)CO)c1CN(CC(F)F)C[C@H](C)O2 ; Cc1nn(C)c2c1-c1cc3c(n[nH]c3c(C)n1)/C=C/c1c(OCC#N)nn([C@@H](C)CO)c1CN(C1CC1)C[C@H](C)O2 ; Cc1nn(C)c2c1-c1cc3c(n[nH]c3c(C)n1)/C=C/c1c(OCC#N)nn([C@@H](C)CO)c1CN(C1CC1)[C@H](C)CO2 ; CCOc1nn([C@@H](C)CO)c2c1/C=C/c1n[nH]c3c(C)nc(cc13)-c1c(COC)nn(C)c1O[C@@H](C)CN(C1CC1)C2] 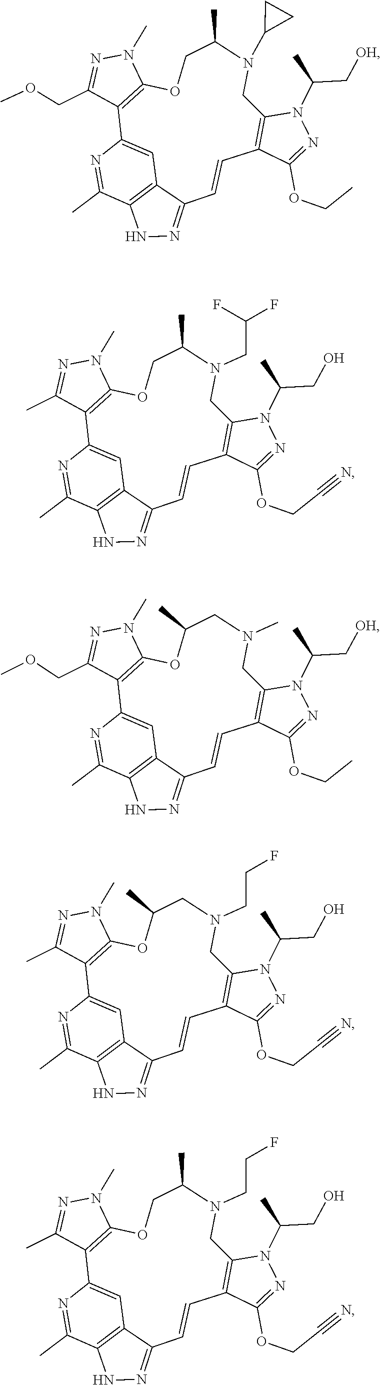 [SMILES: CCOc1nn([C@@H](C)CO)c2c1/C=C/c1n[nH]c3c(C)nc(cc13)-c1c(COC)nn(C)c1OC[C@@H](C)N(C1CC1)C2 ; Cc1nn(C)c2c1-c1cc3c(n[nH]c3c(C)n1)/C=C/c1c(OCC#N)nn([C@@H](C)CO)c1CN(CC(F)F)[C@H](C)CO2 ; CCOc1nn([C@@H](C)CO)c2c1/C=C/c1n[nH]c3c(C)nc(cc13)-c1c(COC)nn(C)c1O[C@@H](C)CN(C)C2 ; Cc1nn(C)c2c1-c1cc3c(n[nH]c3c(C)n1)/C=C/c1c(OCC#N)nn([C@@H](C)CO)c1CN(CCF)C[C@H](C)O2 ; Cc1nn(C)c2c1-c1cc3c(n[nH]c3c(C)n1)/C=C/c1c(OCC#N)nn([C@@H](C)CO)c1CN(CCF)[C@H](C)CO2] 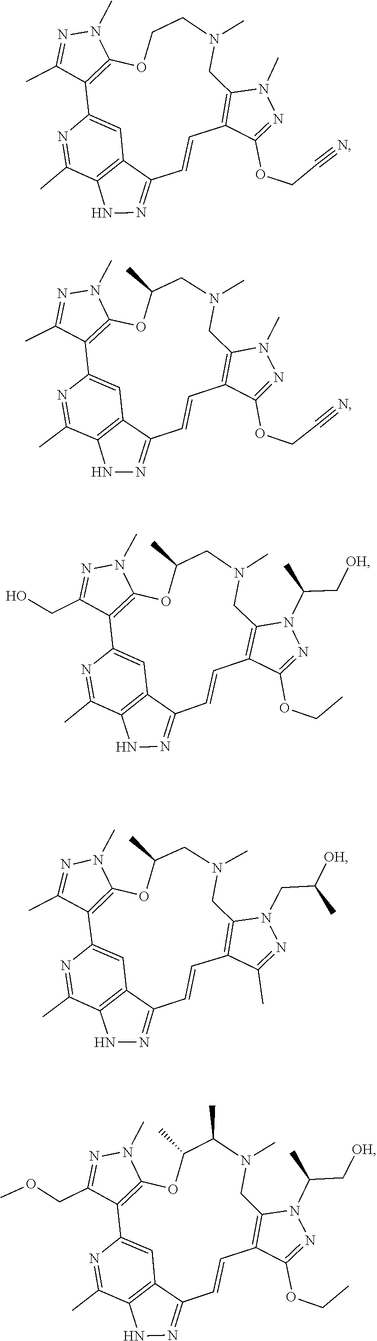 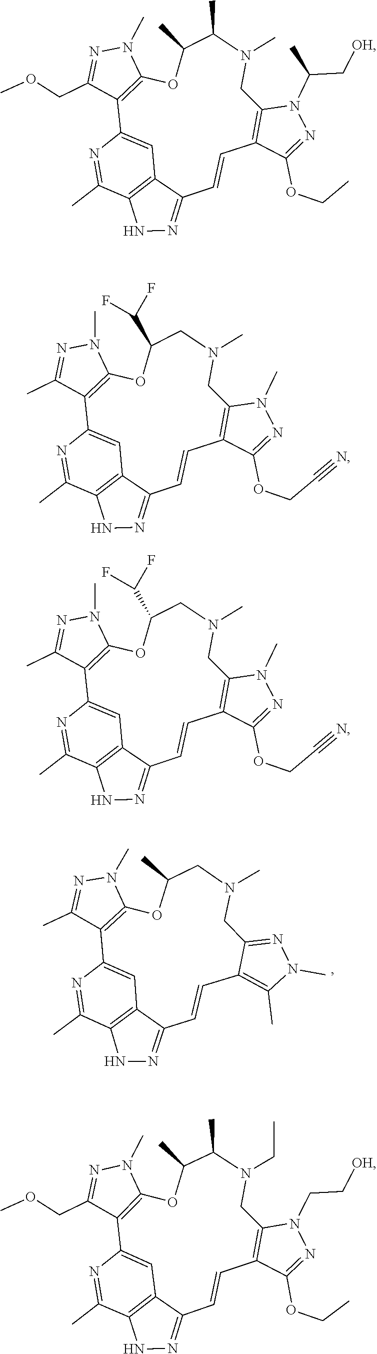 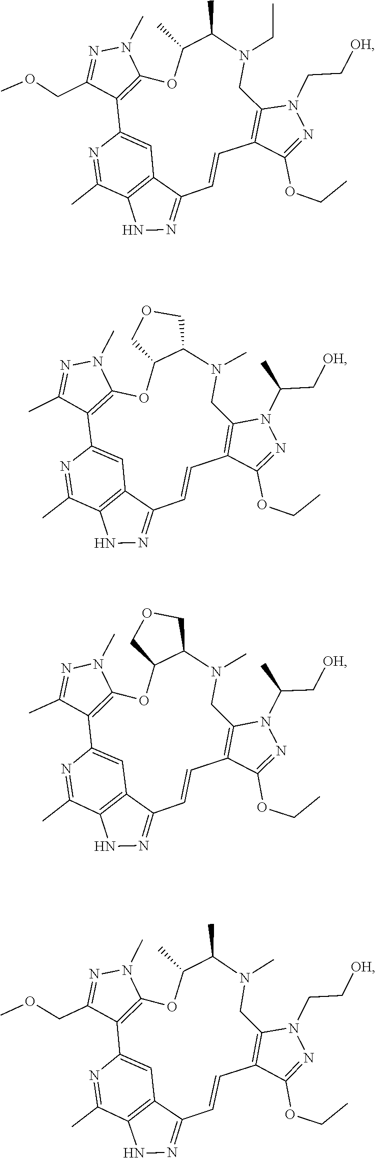 [SMILES: CCOc1nn(CCO)c2c1/C=C/c1n[nH]c3c(C)nc(cc13)-c1c(COC)nn(C)c1O[C@H](C)[C@@H](C)N(CC)C2 ; CCOc1nn([C@@H](C)CO)c2c1/C=C/c1n[nH]c3c(C)nc(cc13)-c1c(C)nn(C)c1O[C@@H]1COC[C@@H]1N(C)C2 ; CCOc1nn([C@@H](C)CO)c2c1/C=C/c1n[nH]c3c(C)nc(cc13)-c1c(C)nn(C)c1O[C@H]1COC[C@H]1N(C)C2 ; CCOc1nn(CCO)c2c1/C=C/c1n[nH]c3c(C)nc(cc13)-c1c(COC)nn(C)c1O[C@H](C)[C@@H](C)N(C)C2] 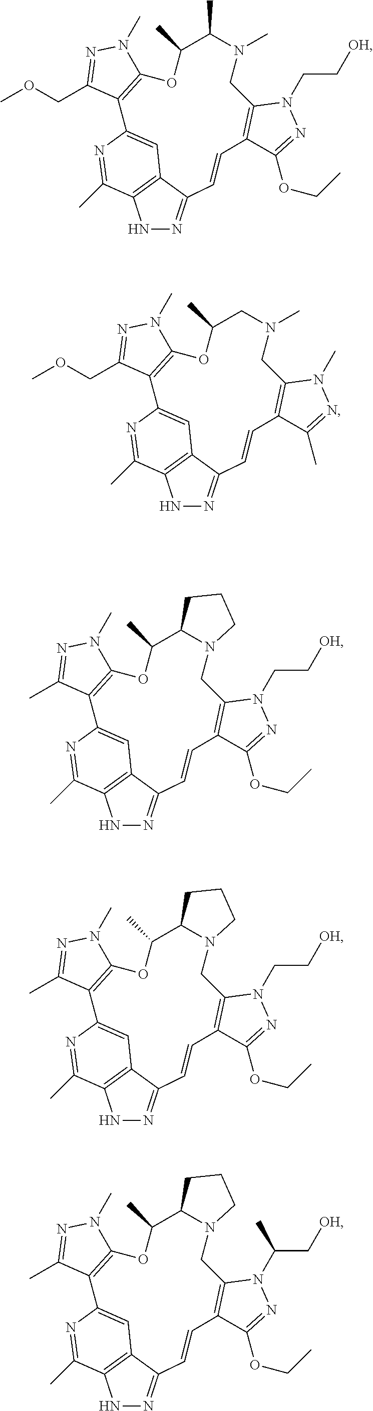 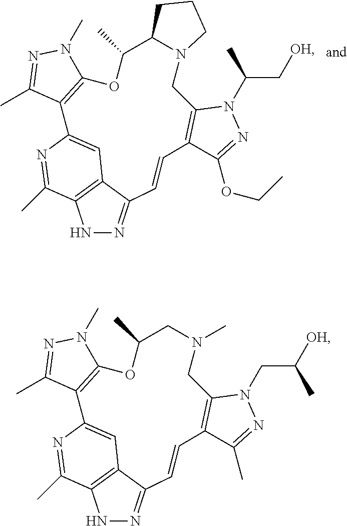 [SMILES: CCOc1nn([C@@H](C)CO)c2c1/C=C/c1n[nH]c3c(C)nc(cc13)-c1c(C)nn(C)c1O[C@H](C)[C@H]1CCCN1C2 ; Cc1nn(C[C@H](C)O)c2c1/C=C/c1n[nH]c3c(C)nc(cc13)-c1c(C)nn(C)c1O[C@@H](C)CN(C)C2]
or a pharmaceutically acceptable salt, solvate, hydrate, or cocrystal thereof.

26. A pharmaceutical composition comprising a compound of any one of the preceding claims, or a pharmaceutically acceptable salt, solvate, hydrate, or cocrystal thereof, and optionally one or more excipients.

27. A method of treating disease in a subject comprising, administering a therapeutically effective amount of a compound of any one of claims 1 to 25, or a pharmaceutically acceptable salt, solvate, hydrate, or cocrystal thereof, or a pharmaceutical composition of claim 26.

28. A compound according to any one of claims 1 to 25, or a pharmaceutically acceptable salt, solvate, hydrate, or cocrystal thereof, for use in a method of treating disease in a subject.

29. Use of a compound according to any one of claims 1 to 25, or a pharmaceutically acceptable salt, solvate, hydrate, or cocrystal thereof, in the manufacture of a medicament for the treatment of disease in a subject.
